SEQUENCE LISTING (266 sequences)

SEQ ID NO: 1 - protein, 700 aa, synthetic construct
  source 1..700: note = V9 (scFab)-Fc(hole) P329G LALA
DIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECSGGGSGGGSEGGGSEGGGSEGGGSEGGGSGGGSGEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 2 - DNA, 2103 bp, synthetic construct
  source 1..2103: mol_type = other DNA; note = V9 (scFab)-Fc(hole) P329G LALA
gacatccagatgacccagagcccctctagcctgagcgccagcgtgggcgacagagtgaccatcacctgtcgggccagccaggacatcagaaactacctgaactggtatcagcagaagcccggcaaggcccccaagctgctgatctactacacctctagactggaaagcggcgtgcccagccggtttagcggcagcggctccggcaccgactacaccctgaccatcagcagcctgcagcccgaggacttcgccacctactactgccagcagggcaacacactcccctggaccttcggccagggcaccaaggtggagatcaagcgtacggtggccgctcccagcgtgttcatcttcccccccagcgacgagcagctgaagtccggcaccgccagcgtcgtgtgcctgctgaacaacttctacccccgggaggccaaggtgcagtggaaggtggacaacgccctgcagagcggcaacagccaggaaagcgtcaccgagcaggacagcaaggactccacctacagcctgagcagcaccctgaccctgagcaaggccgactacgagaagcacaaggtgtacgcctgcgaagtgacccaccagggcctgtccagccccgtgaccaagagcttcaaccggggcgagtgcagcggcggaggctctggaggcggctctgaaggcggaggaagtgagggcggaggctcagaaggcggcggaagcgaaggtggcggctctggcggcggatccggcgaggtgcagctggtcgagtccggcggaggcctggtgcagcctggcggcagcctgagactgagctgcgccgccagcggctacagcttcaccggctacaccatgaactgggtccggcaggctcctggcaagggcctcgaatgggtggccctgatcaacccctacaagggcgtgagcacctacaaccagaagttcaaggaccggttcaccatcagcgtggacaagagcaagaacaccgcctatctgcagatgaacagcctgcgggccgaggacaccgccgtgtactactgcgccagaagcggctactacggcgacagcgactggtacttcgacgtgtggggccagggcacactggtcaccgtgtccagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagctgcagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtgcaccctgcccccatcccgggatgagctgaccaagaaccaggtcagcctctcgtgcgcagtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 3 - protein, 442 aa, synthetic construct
  source 1..442: note = LC007 (VH-CH1)-Fc(knob) P329G LALA
EVQLQESGPGLVKPSQSLSLTCSVTGYSITSGYYWNWIRQFPGNKLEWMGYITYDGSNNYNPSLKNRISITRDTSKNQFFLKLNSVTTEDTATYYCADFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - DNA, 1329 bp, synthetic construct
  source 1..1329: mol_type = other DNA; note = LC007 (VH-CH1)-Fc(knob) P329G LALA
gaggtccagctgcaggagtcaggacctggcctcgtgaaaccttctcagtctctgtctctcacctgctctgtcactggctactccatcaccagtggttattactggaactggatccggcagtttccaggaaacaagctggaatggatgggctacataacctacgacggtagcaataactacaacccatctctcaaaaatcgaatctccatcactcgtgacacatctaagaaccagtttttcctgaagttgaattctgtgactactgaggacacagctacatattactgtgcggactttgactactggggccaaggcaccactctcacagtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaagacccacacctgtcccccttgccctgcccctgaagctgctggtggcccttccgtgttcctgttccccccaaagcccaaggacaccctgatgatcagccggacccccgaagtgacctgcgtggtggtcgatgtgtcccacgaggaccctgaagtgaagttcaattggtacgtggacggcgtggaagtgcacaatgccaagaccaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtacaccctgcccccatgccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaataa

SEQ ID NO: 5 - protein, 214 aa, synthetic construct
  source 1..214: note = LC007 (VL-CL)
DIVLTQSPSSLSASLGDRVTISCSASQGIRNYLNWYQQRPDGTVKLLIYYTSSLHSGVPSRFSGSGSGTDYSLTISNLEPEDIATYYCQQYSKLPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - DNA, 645 bp, synthetic construct
  source 1..645: mol_type = other DNA; note = LC007 (VL-CL)
gatattgtgctcacacagtctccatcctccctgtctgcctctctgggagacagagtcaccatcagttgcagtgcaagtcagggcattagaaattatttaaactggtatcagcagagaccagatggaactgttaaactcctgatctattacacatcaagtttacactcaggagtcccatcaaggttcagtggcagtgggtctgggacagattattctctcaccatcagcaacctggaacctgaagatattgccacttactattgtcagcagtatagtaagcttccttggacgttcggtggaggcaccaagctggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 7 - protein, 704 aa, synthetic construct
  source 1..704: note = LC007 (scFab)-Fc(hole) P329G LALA
DIVLTQSPSSLSASLGDRVTISCSASQGIRNYLNWYQQRPDGTVKLLIYYTSSLHSGVPSRFSGSGSGTDYSLTISNLEPEDIATYYCQQYSKLPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECSGGGSGGGSEGGGSEGGGSEGGGSEGGGSGGGSGEVQLQESGPGLVKPSQSLSLTCSVTGYSITSGYYWNWIRQFPGNKLEWMGYITYDGSNNYNPSLKNRISITRDTSKNQFFLKLNSVTTEDTATYYCADFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSGAQDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 8 - DNA, 2115 bp, synthetic construct
  source 1..2115: mol_type = other DNA; note = LC007 (scFab)-Fc(hole) P329G LALA
gacatcgtgctgacccagagccctagcagcctgagcgccagcctgggcgacagagtgaccatcagctgtagcgcctcccagggcatcagaaactacctgaactggtatcagcagagacccgacggcacagtgaagctgctgatctactacaccagcagcctgcacagcggcgtgccaagcagattcagcggcagcggctccggcacagactacagcctgaccatctccaacctggaacccgaggatatcgccacctactactgccagcagtacagcaagctgccctggaccttcggcggaggcaccaagctggaaatcaagcggaccgtggccgctcccagcgtgttcatcttcccacccagcgacgagcagctgaagtccggcacagccagcgtcgtgtgcctgctgaacaacttctacccccgggaggccaaggtgcagtggaaggtggacaacgccctgcagagcggcaacagccaggaaagcgtcaccgagcaggacagcaaggactccacctacagcctgtccagcaccctgaccctgagcaaggccgactacgagaagcacaaggtgtacgcctgcgaagtgacccaccagggcctgagcagccccgtgaccaagagcttcaaccggggcgagtgtagtggcggaggctctggcggaggaagcgagggcggaggatctgaaggcggcggatctgaggggggaggcagtgaagggggaggctcagggggaggatccggcgaggtgcagctgcaggaatctggccctggcctggtcaagccaagccagagtctgagcctgacctgcagcgtgaccggctacagcattaccagcggctactactggaactggattcggcagttccccggcaataagctggaatggatgggctacatcacctacgacggcagcaacaactacaaccccagcctgaagaaccggatcagcatcacccgggacaccagcaagaaccagttcttcctgaagctgaacagcgtgaccaccgaggacaccgccacatactattgcgccgacttcgactactggggccagggcaccaccctgaccgtgtccagcgccagcacaaagggccctagcgtgttccctctggcccccagcagcaagagcacaagcggcggaacagccgccctgggctgcctcgtgaaggactacttccccgagcccgtgacagtgtcttggaacagcggagccctgacaagcggcgtgcacaccttccctgccgtgctgcagagcagcggcctgtactccctgagcagcgtggtcaccgtgcctagcagcagcctgggcacccagacctacatctgcaacgtgaaccacaagcccagcaacaccaaagtggacaagaaggtggagcccaagagctgtgatggcggaggagggtccggaggcggtggatccggagctcaggacaaaactcacacatgcccaccgtgcccagcacctgaagctgcagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtgcaccctgcccccatcccgggatgagctgaccaagaaccaggtcagcctctcgtgcgcagtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 9 - protein, 466 aa, synthetic construct
  source 1..466: note = V9 (VH-CH1) -Fc(knob) LALA
EVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSGAQDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - DNA, 1401 bp, synthetic construct
  source 1..1401: mol_type = other DNA; note = V9 (VH-CH1) -Fc(knob) LALA
gaggtgcagctggtcgagagcggaggcggcctggtgcagcctggcggcagcctgagactgagctgcgccgccagcggctacagcttcaccggctacaccatgaactgggtccggcaggcacctggcaagggactggaatgggtggccctgatcaacccctacaagggcgtgagcacctacaaccagaagttcaaggaccggttcaccatcagcgtggacaagagcaagaacaccgcctatctgcagatgaacagcctgcgggccgaggacaccgccgtgtactactgcgccagaagcggctactacggcgacagcgactggtacttcgacgtgtggggccagggcaccctcgtgaccgtgtctagcgctagcaccaagggcccctccgtgttccccctggcccccagcagcaagagcaccagcggcggcacagccgctctgggctgcctggtcaaggactacttccccgagcccgtgaccgtgtcctggaacagcggagccctgacctccggcgtgcacaccttccccgccgtgctgcagagttctggcctgtatagcctgagcagcgtggtcaccgtgccttctagcagcctgggcacccagacctacatctgcaacgtgaaccacaagcccagcaacaccaaggtggacaagaaggtggagcccaagagctgcgacggcggtggtggctccggaggcggtggatccggagctcaggacaaaactcacacatgcccaccgtgcccagcacctgaagctgcagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatgccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 11 - protein, 214 aa, synthetic construct
  source 1..214: note = V9 (VL-CL)
DIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - DNA, 645 bp, synthetic construct
  source 1..645: mol_type = other DNA; note = V9 (VL-CL)
gacatccagatgacccagagccccagcagcctgagcgccagcgtgggcgacagagtgaccatcacctgtcgggccagccaggacatcagaaactacctgaactggtatcagcagaagcccggcaaggcccccaagctgctgatctactacacctctagactggaaagcggcgtgcccagccggtttagcggcagcggctccggcaccgactacaccctgaccatcagcagcctgcagcccgaggacttcgccacctactactgccagcagggcaacacactgccctggaccttcggccagggcacaaaggtggagatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 13 - protein, 926 aa, synthetic construct [sequence omitted]

SEQ ID NO: 14 - DNA, 2781 bp, synthetic construct
  source 1..2781: mol_type = other DNA; note = V9 (scFab)-LC007 (VH-CH1)-Fc(knob) wt
gacatccagatgacccagagcccctctagcctgagcgccagcgtgggcgacagagtgaccatcacctgtcgggccagccaggacatcagaaactacctgaactggtatcagcagaagcccggcaaggcccccaagctgctgatctactacacctctagactggaaagcggcgtgcccagccggtttagcggcagcggctccggcaccgactacaccctgaccatcagcagcctgcagcccgaggacttcgccacctactactgccagcagggcaacacactcccctggaccttcggccagggcaccaaggtggagatcaagcgtacggtggccgctcccagcgtgttcatcttcccccccagcgacgagcagctgaagtccggcaccgccagcgtcgtgtgcctgctgaacaacttctacccccgggaggccaaggtgcagtggaaggtggacaacgccctgcagagcggcaacagccaggaaagcgtcaccgagcaggacagcaaggactccacctacagcctgagcagcaccctgaccctgagcaaggccgactacgagaagcacaaggtgtacgcctgcgaagtgacccaccagggcctgtccagccccgtgaccaagagcttcaaccggggcgagtgcagcggcggaggctctggaggcggctctgaaggcggaggaagtgagggcggaggctcagaaggcggcggaagcgaaggtggcggctctggcggcggatccggcgaggtgcagctggtcgagtccggcggaggcctggtgcagcctggcggcagcctgagactgagctgcgccgccagcggctacagcttcaccggctacaccatgaactgggtccggcaggctcctggcaagggcctcgaatgggtggccctgatcaacccctacaagggcgtgagcacctacaaccagaagttcaaggaccggttcaccatcagcgtggacaagagcaagaacaccgcctatctgcagatgaacagcctgcgggccgaggacaccgccgtgtactactgcgccagaagcggctactacggcgacagcgactggtacttcgacgtgtggggccagggcacactggtcaccgtgtccagcgctagcaccaagggcccctccgtgttccccctggcccccagcagcaagagcaccagcggcggcacagccgccctcggctgcctggtcaaggactacttccccgagcccgtgaccgtgtcctggaacagcggagccctgacctccggcgtgcacaccttccccgccgtgctgcagagcagcggcctgtacagcctgtccagcgtggtcaccgtgccctccagcagcctgggcacccagacctacatctgcaacgtgaaccacaagcccagcaataccaaggtggacaagaaggtggagcccaagagctgcgacggcggtggtggctccggaggcggtggatctgaagtgcagctgcaggaaagcggccctggcctggtcaagcccagccagagcctgagcctgacctgtagcgtgaccggctactccatcacctccggctactactggaattggattcggcagttccccggcaacaagctggaatggatgggctacatcacctacgacggcagcaacaactacaaccccagcctgaagaaccggatcagcatcacccgggacaccagcaagaaccagttcttcctgaagttgaattctgtgactactgaggacacagctacatattactgtgcggactttgactactggggccaaggcaccactctcacagtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatgccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 15 - protein, 442 aa, synthetic construct
  source 1..442: note = LC007 (VH-CH1)-Fc(hole) wt
EVQLQESGPGLVKPSQSLSLTCSVTGYSITSGYYWNWIRQFPGNKLEWMGYITYDGSNNYNPSLKNRISITRDTSKNQFFLKLNSVTTEDTATYYCADFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 16 - DNA, 1329 bp, synthetic construct
  source 1..1329: mol_type = other DNA; note = LC007 (VH-CH1)-Fc(hole) wt
gaggtccagctgcaggagtcaggacctggcctcgtgaaaccttctcagtctctgtctctcacctgctctgtcactggctactccatcaccagtggttattactggaactggatccggcagtttccaggaaacaagctggaatggatgggctacataacctacgacggtagcaataactacaacccatctctcaaaaatcgaatctccatcactcgtgacacatctaagaaccagtttttcctgaagttgaattctgtgactactgaggacacagctacatattactgtgcggactttgactactggggccaaggcaccactctcacagtctcctcagctagcaccaagggcccaagcgtgttccctctggcccccagcagcaagagcacaagcggcggaacagccgccctgggctgcctggtcaaggactacttccccgagcccgtgacagtgtcctggaacagcggagccctgaccagcggcgtgcacacctttccagccgtgctgcagagcagcggcctgtacagcctgagcagcgtggtcacagtgcctagcagcagcctgggcacccagacctacatctgcaacgtgaaccacaagcccagcaacaccaaggtggacaagaaggtggagcccaagagctgcgacaagacccacacctgtcccccttgtcctgcccctgagctgctgggcggacccagcgtgttcctgttccccccaaagcccaaggacaccctgatgatcagccggacccccgaagtgacctgcgtggtggtggacgtgtcccacgaggaccctgaagtgaagttcaattggtacgtggacggcgtggaggtgcacaatgccaagaccaagccccgggaggaacagtacaacagcacctaccgggtggtgtccgtgctgaccgtgctgcaccaggactggctgaacggcaaagagtacaagtgcaaggtctccaacaaggccctgcctgcccccatcgagaaaaccatcagcaaggccaagggccagcccagagaaccccaggtgtgcaccctgccccccagcagagatgagctgaccaagaaccaggtgtccctgagctgtgccgtcaagggcttctaccccagcgatatcgccgtggagtgggagagcaacggccagcctgagaacaactacaagaccaccccccctgtgctggacagcgacggcagcttcttcctggtgtccaaactgaccgtggacaagagccggtggcagcagggcaacgtgttcagctgcagcgtgatgcacgaggccctgcacaaccactacacccagaagtccctgagcctgagccccggcaagtga

SEQ ID NO: 17 - protein, 926 aa, synthetic construct
  source 1..926: note = V9 (scFab)-LC007 (VH-CH1)-Fc(knob) LALA
DIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECSGGGSGGGSEGGGSEGGGSEGGGSEGGGSGGGSGEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLQESGPGLVKPSQSLSLTCSVTGYSITSGYYWNWIRQFPGNKLEWMGYITYDGSNNYNPSLKNRISITRDTSKNQFFLKLNSVTTEDTATYYCADFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - DNA, 2781 bp, synthetic construct
  source 1..2781: mol_type = other DNA; note = V9 (scFab)-LC007 (VH-CH1)-Fc(knob) LALA
gacatccagatgacccagagcccctctagcctgagcgccagcgtgggcgacagagtgaccatcacctgtcgggccagccaggacatcagaaactacctgaactggtatcagcagaagcccggcaaggcccccaagctgctgatctactacacctctagactggaaagcggcgtgcccagccggtttagcggcagcggctccggcaccgactacaccctgaccatcagcagcctgcagcccgaggacttcgccacctactactgccagcagggcaacacactcccctggaccttcggccagggcaccaaggtggagatcaagcgtacggtggccgctcccagcgtgttcatcttcccccccagcgacgagcagctgaagtccggcaccgccagcgtcgtgtgcctgctgaacaacttctacccccgggaggccaaggtgcagtggaaggtggacaacgccctgcagagcggcaacagccaggaaagcgtcaccgagcaggacagcaaggactccacctacagcctgagcagcaccctgaccctgagcaaggccgactacgagaagcacaaggtgtacgcctgcgaagtgacccaccagggcctgtccagccccgtgaccaagagcttcaaccggggcgagtgcagcggcggaggctctggaggcggctctgaaggcggaggaagtgagggcggaggctcagaaggcggcggaagcgaaggtggcggctctggcggcggatccggcgaggtgcagctggtcgagtccggcggaggcctggtgcagcctggcggcagcctgagactgagctgcgccgccagcggctacagcttcaccggctacaccatgaactgggtccggcaggctcctggcaagggcctcgaatgggtggccctgatcaacccctacaagggcgtgagcacctacaaccagaagttcaaggaccggttcaccatcagcgtggacaagagcaagaacaccgcctatctgcagatgaacagcctgcgggccgaggacaccgccgtgtactactgcgccagaagcggctactacggcgacagcgactggtacttcgacgtgtggggccagggcacactggtcaccgtgtccagcgctagcaccaagggcccctccgtgttccccctggcccccagcagcaagagcaccagcggcggcacagccgccctcggctgcctggtcaaggactacttccccgagcccgtgaccgtgtcctggaacagcggagccctgacctccggcgtgcacaccttccccgccgtgctgcagagcagcggcctgtacagcctgtccagcgtggtcaccgtgccctccagcagcctgggcacccagacctacatctgcaacgtgaaccacaagcccagcaataccaaggtggacaagaaggtggagcccaagagctgcgacggcggtggtggctccggaggcggtggatctgaagtgcagctgcaggaaagcggccctggcctggtcaagcccagccagagcctgagcctgacctgtagcgtgaccggctactccatcacctccggctactactggaattggattcggcagttccccggcaacaagctggaatggatgggctacatcacctacgacggcagcaacaactacaaccccagcctgaagaaccggatcagcatcacccgggacaccagcaagaaccagttcttcctgaagttgaattctgtgactactgaggacacagctacatattactgtgcggactttgactactggggccaaggcaccactctcacagtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagctgcagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatgccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 19 - protein, 442 aa, synthetic construct
  source 1..442: note = LC007 (VH-CH1)-Fc(hole) LALA
EVQLQESGPGLVKPSQSLSLTCSVTGYSITSGYYWNWIRQFPGNKLEWMGYITYDGSNNYNPSLKNRISITRDTSKNQFFLKLNSVTTEDTATYYCADFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 20 - DNA, 1329 bp, synthetic construct
  source 1..1329: mol_type = other DNA; note = LC007 (VH-CH1)-Fc(hole) LALA
gaggtccagctgcaggagtcaggacctggcctcgtgaaaccttctcagtctctgtctctcacctgctctgtcactggctactccatcaccagtggttattactggaactggatccggcagtttccaggaaacaagctggaatggatgggctacataacctacgacggtagcaataactacaacccatctctcaaaaatcgaatctccatcactcgtgacacatctaagaaccagtttttcctgaagttgaattctgtgactactgaggacacagctacatattactgtgcggactttgactactggggccaaggcaccactctcacagtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagctgcagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtgcaccctgcccccatcccgggatgagctgaccaagaaccaggtcagcctctcgtgcgcagtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 21 - protein, 926 aa, synthetic construct
  source 1..926: note = V9 (scFab)-LC007 (VH-CH1)-Fc(knob) P329G LALA
DIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECSGGGSGGGSEGGGSEGGGSEGGGSEGGGSGGGSGEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLQESGPGLVKPSQSLSLTCSVTGYSITSGYYWNWIRQFPGNKLEWMGYITYDGSNNYNPSLKNRISITRDTSKNQFFLKLNSVTTEDTATYYCADFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - DNA, 2781 bp, synthetic construct
  source 1..2781: mol_type = other DNA; note = V9 (scFab)-LC007 (VH-CH1)-Fc(knob) P329G LALA
gacatccagatgacccagagcccctctagcctgagcgccagcgtgggcgacagagtgaccatcacctgtcgggccagccaggacatcagaaactacctgaactggtatcagcagaagcccggcaaggcccccaagctgctgatctactacacctctagactggaaagcggcgtgcccagccggtttagcggcagcggctccggcaccgactacaccctgaccatcagcagcctgcagcccgaggacttcgccacctactactgccagcagggcaacacactcccctggaccttcggccagggcaccaaggtggagatcaagcgtacggtggccgctcccagcgtgttcatcttcccccccagcgacgagcagctgaagtccggcaccgccagcgtcgtgtgcctgctgaacaacttctacccccgggaggccaaggtgcagtggaaggtggacaacgccctgcagagcggcaacagccaggaaagcgtcaccgagcaggacagcaaggactccacctacagcctgagcagcaccctgaccctgagcaaggccgactacgagaagcacaaggtgtacgcctgcgaagtgacccaccagggcctgtccagccccgtgaccaagagcttcaaccggggcgagtgcagcggcggaggctctggaggcggctctgaaggcggaggaagtgagggcggaggctcagaaggcggcggaagcgaaggtggcggctctggcggcggatccggcgaggtgcagctggtcgagtccggcggaggcctggtgcagcctggcggcagcctgagactgagctgcgccgccagcggctacagcttcaccggctacaccatgaactgggtccggcaggctcctggcaagggcctcgaatgggtggccctgatcaacccctacaagggcgtgagcacctacaaccagaagttcaaggaccggttcaccatcagcgtggacaagagcaagaacaccgcctatctgcagatgaacagcctgcgggccgaggacaccgccgtgtactactgcgccagaagcggctactacggcgacagcgactggtacttcgacgtgtggggccagggcacactggtcaccgtgtccagcgctagcaccaagggcccctccgtgttccccctggcccccagcagcaagagcaccagcggcggcacagccgccctcggctgcctggtcaaggactacttccccgagcccgtgaccgtgtcctggaacagcggagccctgacctccggcgtgcacaccttccccgccgtgctgcagagcagcggcctgtacagcctgtccagcgtggtcaccgtgccctccagcagcctgggcacccagacctacatctgcaacgtgaaccacaagcccagcaataccaaggtggacaagaaggtggagcccaagagctgcgacggcggtggtggctccggaggcggtggatctgaagtgcagctgcaggaaagcggccctggcctggtcaagcccagccagagcctgagcctgacctgtagcgtgaccggctactccatcacctccggctactactggaattggattcggcagttccccggcaacaagctggaatggatgggctacatcacctacgacggcagcaacaactacaaccccagcctgaagaaccggatcagcatcacccgggacaccagcaagaaccagttcttcctgaagttgaattctgtgactactgaggacacagctacatattactgtgcggactttgactactggggccaaggcaccactctcacagtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagctgcagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtacaccctgcccccatgccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 23 - protein, 442 aa, synthetic construct
  source 1..442: note = LC007 (VH-CH1)-Fc(hole) P329G LALA
EVQLQESGPGLVKPSQSLSLTCSVTGYSITSGYYWNWIRQFPGNKLEWMGYITYDGSNNYNPSLKNRISITRDTSKNQFFLKLNSVTTEDTATYYCADFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 24 - DNA, 1329 bp, synthetic construct
  source 1..1329: mol_type = other DNA; note = LC007 (VH-CH1)-Fc(hole) P329G LALA
gaggtccagctgcaggagtcaggacctggcctcgtgaaaccttctcagtctctgtctctcacctgctctgtcactggctactccatcaccagtggttattactggaactggatccggcagtttccaggaaacaagctggaatggatgggctacataacctacgacggtagcaataactacaacccatctctcaaaaatcgaatctccatcactcgtgacacatctaagaaccagtttttcctgaagttgaattctgtgactactgaggacacagctacatattactgtgcggactttgactactggggccaaggcaccactctcacagtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagctgcagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtgcaccctgcccccatcccgggatgagctgaccaagaaccaggtcagcctctcgtgcgcagtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 25 - protein, 926 aa, synthetic construct
  source 1..926: note = V9 (scFab)-LC007 (VH-CH1)-Fc(knob) P329G LALA N297D
DIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECSGGGSGGGSEGGGSEGGGSEGGGSEGGGSGGGSGEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLQESGPGLVKPSQSLSLTCSVTGYSITSGYYWNWIRQFPGNKLEWMGYITYDGSNNYNPSLKNRISITRDTSKNQFFLKLNSVTTEDTATYYCADFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYDSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - DNA, 2781 bp, synthetic construct
  source 1..2781: mol_type = other DNA; note = V9 (scFab)-LC007 (VH-CH1)-Fc(knob) P329G LALA N297D
gacatccagatgacccagagcccctctagcctgagcgccagcgtgggcgacagagtgaccatcacctgtcgggccagccaggacatcagaaactacctgaactggtatcagcagaagcccggcaaggcccccaagctgctgatctactacacctctagactggaaagcggcgtgcccagccggtttagcggcagcggctccggcaccgactacaccctgaccatcagcagcctgcagcccgaggacttcgccacctactactgccagcagggcaacacactcccctggaccttcggccagggcaccaaggtggagatcaagcgtacggtggccgctcccagcgtgttcatcttcccccccagcgacgagcagctgaagtccggcaccgccagcgtcgtgtgcctgctgaacaacttctacccccgggaggccaaggtgcagtggaaggtggacaacgccctgcagagcggcaacagccaggaaagcgtcaccgagcaggacagcaaggactccacctacagcctgagcagcaccctgaccctgagcaaggccgactacgagaagcacaaggtgtacgcctgcgaagtgacccaccagggcctgtccagccccgtgaccaagagcttcaaccggggcgagtgcagcggcggaggctctggaggcggctctgaaggcggaggaagtgagggcggaggctcagaaggcggcggaagcgaaggtggcggctctggcggcggatccggcgaggtgcagctggtcgagtccggcggaggcctggtgcagcctggcggcagcctgagactgagctgcgccgccagcggctacagcttcaccggctacaccatgaactgggtccggcaggctcctggcaagggcctcgaatgggtggccctgatcaacccctacaagggcgtgagcacctacaaccagaagttcaaggaccggttcaccatcagcgtggacaagagcaagaacaccgcctatctgcagatgaacagcctgcgggccgaggacaccgccgtgtactactgcgccagaagcggctactacggcgacagcgactggtacttcgacgtgtggggccagggcacactggtcaccgtgtccagcgctagcaccaagggcccctccgtgttccccctggcccccagcagcaagagcaccagcggcggcacagccgccctcggctgcctggtcaaggactacttccccgagcccgtgaccgtgtcctggaacagcggagccctgacctccggcgtgcacaccttccccgccgtgctgcagagcagcggcctgtacagcctgtccagcgtggtcaccgtgccctccagcagcctgggcacccagacctacatctgcaacgtgaaccacaagcccagcaataccaaggtggacaagaaggtggagcccaagagctgcgacggcggtggtggctccggaggcggtggatctgaagtgcagctgcaggaaagcggccctggcctggtcaagcccagccagagcctgagcctgacctgtagcgtgaccggctactccatcacctccggctactactggaattggattcggcagttccccggcaacaagctggaatggatgggctacatcacctacgacggcagcaacaactacaaccccagcctgaagaaccggatcagcatcacccgggacaccagcaagaaccagttcttcctgaagttgaattctgtgactactgaggacacagctacatattactgtgcggactttgactactggggccaaggcaccactctcacagtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagctgcagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtacaccctgcccccatgccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 27 - protein, 442 aa, synthetic construct
  source 1..442: note = LC007 (VH-CH1)-Fc(hole) P329G LALA N297D
EVQLQESGPGLVKPSQSLSLTCSVTGYSITSGYYWNWIRQFPGNKLEWMGYITYDGSNNYNPSLKNRISITRDTSKNQFFLKLNSVTTEDTATYYCADFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYDSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 28 - DNA, 1329 bp, synthetic construct
  source 1..1329: mol_type = other DNA; note = LC007 (VH-CH1)-Fc(hole) P329G LALA N297D
gaggtccagctgcaggagtcaggacctggcctcgtgaaaccttctcagtctctgtctctcacctgctctgtcactggctactccatcaccagtggttattactggaactggatccggcagtttccaggaaacaagctggaatggatgggctacataacctacgacggtagcaataactacaacccatctctcaaaaatcgaatctccatcactcgtgacacatctaagaaccagtttttcctgaagttgaattctgtgactactgaggacacagctacatattactgtgcggactttgactactggggccaaggcaccactctcacagtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagctgcagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtgcaccctgcccccatcccgggatgagctgaccaagaaccaggtcagcctctcgtgcgcagtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 29 - protein, 664 aa, synthetic construct
  source 1..664: note = V9 (VL-CH1)-LC007 (VH-CH1)-Fc(hole) P329G LALA
DIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSEVQLQESGPGLVKPSQSLSLTCSVTGYSITSGYYWNWIRQFPGNKLEWMGYITYDGSNNYNPSLKNRISITRDTSKNQFFLKLNSVTTEDTATYYCADFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 30 - DNA, 1995 bp, synthetic construct
  source 1..1995: mol_type = other DNA; note = V9 (VL-CH1)-LC007 (VH-CH1)-Fc(hole) P329G LALA
gatatccagatgacccagagccccagctctctgagcgccagcgtgggcgacagagtgaccatcacctgtcgggccagccaggacatcagaaactacctgaactggtatcagcagaagcccggcaaggcccccaagctgctgatctactacaccagcagactggaaagcggcgtgccctccagattttccggcagcggctccggcaccgactacaccctgaccatcagcagcctgcagcccgaggatttcgccacatattactgccagcagggcaataccctgccctggaccttcggacagggcacaaaagtggaaatcaagagcagcgcttccaccaaaggcccttccgtgtttcctctggctcctagctccaagtccacctctggaggcaccgctgctctcggatgcctcgtgaaggattattttcctgagcctgtgacagtgtcctggaatagcggagcactgacctctggagtgcatactttccccgctgtgctgcagtcctctggactgtacagcctgagcagcgtggtgacagtgcccagcagcagcctgggcacccagacctacatctgcaacgtgaaccacaagcccagcaacaccaaggtggacaagaaggtggaacccaagtcttgtggcggaggcggatccggcggagggggatctgaggtgcagctgcaggaaagcggccctggcctggtgaaacccagccagagcctgagcctgacctgcagcgtgaccggctacagcatcaccagcggctactactggaactggatcagacagttccccggcaacaagctggaatggatgggctacatcacctacgacggcagcaacaactacaaccccagcctgaagaacagaatcagcatcacccgggacaccagcaagaaccagttcttcctgaagctgaacagcgtgaccaccgaggacaccgccacctactactgcgccgacttcgactactggggccagggcaccaccctgaccgtgtcctccgcctctaccaagggccccagcgtgttccccctggcacccagcagcaagagcacatctggcggaacagccgctctgggctgtctggtgaaagactacttccccgagcccgtgaccgtgtcttggaactctggcgccctgaccagcggcgtgcacacctttccagccgtgctgcagagcagcggcctgtactccctgtcctccgtggtcaccgtgccctctagctccctgggaacacagacatatatctgtaatgtcaatcacaagccttccaacaccaaagtcgataagaaagtcgagcccaagagctgcgacaaaactcacacatgcccaccgtgcccagcacctgaagctgcagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtgcaccctgcccccatcccgggatgagctgaccaagaaccaggtcagcctctcgtgcgcagtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 31 - protein, 229 aa, synthetic construct
  source 1..229: note = Fc(knob) wt
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKSG

SEQ ID NO: 32 - DNA, 690 bp, synthetic construct
  source 1..690: mol_type = other DNA; note = Fc(knob) wt
gacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatgccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatccggatga

SEQ ID NO: 33 - protein, 229 aa, synthetic construct
  source 1..229: note = V9 (VH-CL)
EVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - DNA, 690 bp, synthetic construct
  source 1..690: mol_type = other DNA; note = V9 (VH-CL)
gaggtgcagctggtcgagagcggaggcggcctggtgcagcctggcggcagcctgagactgagctgcgccgccagcggctacagcttcaccggctacaccatgaactgggtccggcaggcacctggcaagggactggaatgggtggccctgatcaacccctacaagggcgtgagcacctacaaccagaagttcaaggaccggttcaccatcagcgtggacaagagcaagaacaccgcctatctgcagatgaacagcctgcgggccgaggacaccgccgtgtactactgcgccagaagcggctactacggcgacagcgactggtacttcgacgtgtggggccagggcaccctcgtgaccgtgtctagcgctagcgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 35 - protein, 670 aa, synthetic construct
  source 1..670: note = FN18 (VL-CH1)-LC007 (VH-CH1)-Fc(hole) P329G LALA
DIVMSQSPSSLAVSVGEKVTMSCKSSQSLLYSSNQKNYLAWYQQKPGQSPKLLINWASTRESGVPDRFTGSGSRTDFTLTISSVKAEDLAVYFCQQFYSYPPTFGGGTKLEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSEVQLQESGPGLVKPSQSLSLTCSVTGYSITSGYYWNWIRQFPGNKLEWMGYITYDGSNNYNPSLKNRISITRDTSKNQFFLKLNSVTTEDTATYYCADFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 36 - DNA, 2013 bp, synthetic construct
  source 1..2013: mol_type = other DNA; note = FN18 (VL-CH1)-LC007 (VH-CH1)-Fc(hole) P329G LALA
gacatcgtgatgagccagagccccagcagcctggccgtgtccgtgggcgagaaagtgaccatgagctgcaagagcagccagagcctgctgtactcctctaaccagaagaactacctggcctggtatcagcagaagcccggccagtcccccaagctgctgatcaactgggccagcacccgcgagagcggcgtgcccgatagattcacaggcagcggcagccggaccgacttcaccctgaccatcagcagcgtgaaggccgaggatctggccgtgtacttctgccagcagttctacagctacccccccaccttcggcggaggcacgaagctggaaatcaagagcagcgcttccaccaaaggcccttccgtgtttcctctggctcctagctccaagtccacctctggaggcaccgctgctctcggatgcctcgtgaaggattattttcctgagcctgtgacagtgtcctggaatagcggagcactgacctctggagtgcatactttccccgctgtgctgcagtcctctggactgtacagcctgagcagcgtggtgacagtgcccagcagcagcctgggcacccagacctacatctgcaacgtgaaccacaagcccagcaacaccaaggtggacaagaaggtggaacccaagtcttgtggcggaggcggatccggcggagggggatctgaggtgcagctgcaggaaagcggccctggcctggtgaaacccagccagagcctgagcctgacctgcagcgtgaccggctacagcatcaccagcggctactactggaactggatcagacagttccccggcaacaagctggaatggatgggctacatcacctacgacggcagcaacaactacaaccccagcctgaagaacagaatcagcatcacccgggacaccagcaagaaccagttcttcctgaagctgaacagcgtgaccaccgaggacaccgccacctactactgcgccgacttcgactactggggccagggcaccaccctgaccgtgtcctccgcctctaccaagggccccagcgtgttccccctggcacccagcagcaagagcacatctggcggaacagccgctctgggctgtctggtgaaagactacttccccgagcccgtgaccgtgtcttggaactctggcgccctgaccagcggcgtgcacacctttccagccgtgctgcagagcagcggcctgtactccctgtcctccgtggtcaccgtgccctctagctccctgggaacacagacatatatctgtaatgtcaatcacaagccttccaacaccaaagtcgataagaaagtcgagcccaagagctgcgacaaaactcacacatgcccaccgtgcccagcacctgaagctgcagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtgcaccctgcccccatcccgggatgagctgaccaagaaccaggtcagcctctcgtgcgcagtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 37 - protein, 231 aa, synthetic construct
  source 1..231: note = FN18 (VH-CL)
QVQLQQSEAELARPGASVKMSCKASGYTFTDYTIHWLKQRPGQGLDWIGYFNPSSESTEYNRKFKDRTILTADRSSTTAYMQLSSLTSEDSAVYYCSRKGEKLLGNRYWYFDVWGAGTSVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - DNA, 696 bp, synthetic construct
  source 1..696: mol_type = other DNA; note = FN18 (VH-CL)
caggtgcagctgcagcagagcgaggccgagctggctagacctggagccagcgtgaagatgagctgcaaggccagcggctacaccttcaccgactacaccatccactggctgaagcagcggcctggacagggcctggactggatcggctacttcaaccccagcagcgagagcaccgagtacaaccggaagttcaaggaccggaccatcctgaccgccgacagaagcagcaccaccgcctacatgcagctgagcagcctgaccagcgaggacagcgccgtgtactactgcagccggaagggcgagaagctgctgggcaacagatactggtacttcgacgtgtggggagccggcaccagcgtgaccgtgtctagcgctagcgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 39 - protein, 664 aa, synthetic construct
  source 1..664: note = 2C11 (VL-CH1)-LC007 (VH-CH1)-Fc(hole) P329G LALA
DIQMTQSPSSLPASLGDRVTINCQASQDISNYLNWYQQKPGKAPKLLIYYTNKLADGVPSRFSGSGSGRDSSFTISSLESEDIGSYYCQQYYNYPWTFGPGTKLEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSEVQLQESGPGLVKPSQSLSLTCSVTGYSITSGYYWNWIRQFPGNKLEWMGYITYDGSNNYNPSLKNRISITRDTSKNQFFLKLNSVTTEDTATYYCADFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 40 - DNA, 1995 bp, synthetic construct
  source 1..1995: mol_type = other DNA; note = 2C11 (VL-CH1)-LC007 (VH-CH1)-Fc(hole) P329G LALA
gacatccagatgacccagagccccagcagcctgcctgccagcctgggcgacagagtgaccatcaactgccaggccagccaggacatcagcaactacctgaactggtatcagcagaagcctggcaaggcccccaagctgctgatctactacaccaacaagctggccgacggcgtgcccagcagattcagcggcagcggctccggcagagacagcagcttcaccatctccagcctggaaagcgaggacatcggcagctactactgccagcagtactacaactacccctggaccttcggccctggcaccaagctggaaatcaagagcagcgcttccaccaaaggcccttccgtgtttcctctggctcctagctccaagtccacctctggaggcaccgctgctctcggatgcctcgtgaaggattattttcctgagcctgtgacagtgtcctggaatagcggagcactgacctctggagtgcatactttccccgctgtgctgcagtcctctggactgtacagcctgagcagcgtggtgacagtgcccagcagcagcctgggcacccagacctacatctgcaacgtgaaccacaagcccagcaacaccaaggtggacaagaaggtggaacccaagtcttgtggcggaggcggatccggcggagggggatctgaggtgcagctgcaggaaagcggccctggcctggtgaaacccagccagagcctgagcctgacctgcagcgtgaccggctacagcatcaccagcggctactactggaactggatcagacagttccccggcaacaagctggaatggatgggctacatcacctacgacggcagcaacaactacaaccccagcctgaagaacagaatcagcatcacccgggacaccagcaagaaccagttcttcctgaagctgaacagcgtgaccaccgaggacaccgccacctactactgcgccgacttcgactactggggccagggcaccaccctgaccgtgtcctccgcctctaccaagggccccagcgtgttccccctggcacccagcagcaagagcacatctggcggaacagccgctctgggctgtctggtgaaagactacttccccgagcccgtgaccgtgtcttggaactctggcgccctgaccagcggcgtgcacacctttccagccgtgctgcagagcagcggcctgtactccctgtcctccgtggtcaccgtgccctctagctccctgggaacacagacatatatctgtaatgtcaatcacaagccttccaacaccaaagtcgataagaaagtcgagcccaagagctgcgacaaaactcacacatgcccaccgtgcccagcacctgaagctgcagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtgcaccctgcccccatcccgggatgagctgaccaagaaccaggtcagcctctcgtgcgcagtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 41 - protein, 223 aa, synthetic construct
  source 1..223: note = 2C11 (VH-CL)
EVQLVESGGGLVQPGKSLKLSCEASGFTFSGYGMHWVRQAPGRGLESVAYITSSSINIKYADAVKGRFTVSRDNAKNLLFLQMNILKSEDTAMYYCARFDWDKNYWGQGTMVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 42 - DNA, 672 bp, synthetic construct
  source 1..672: mol_type = other DNA; note = 2C11 (VH-CL)
gaggtgcagctggtggaaagcggcggaggcctggtgcagcccggcaagagcctgaagctgagctgcgaggccagcggcttcaccttcagcggctacggcatgcactgggtgagacaggcccctggcagaggactggaaagcgtggcctacatcaccagcagcagcatcaacattaagtacgccgacgccgtgaagggccggttcaccgtgtccagggataacgccaagaacctgctgttcctgcagatgaacatcctgaagtccgaggacaccgctatgtattactgcgccagattcgactgggacaagaactactggggccagggcaccatggtcacagtgtctagcgctagcgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 43 - protein, 700 aa, synthetic construct
  source 1..700: note = V9 (scFab)-Fc(knob) P329G LALA
DIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECSGGGSGGGSEGGGSEGGGSEGGGSEGGGSGGGSGEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - DNA, 2103 bp, synthetic construct
  source 1..2103: mol_type = other DNA; note = V9 (scFab)-Fc(knob) P329G LALA
gacatccagatgacccagagcccctctagcctgagcgccagcgtgggcgacagagtgaccatcacctgtcgggccagccaggacatcagaaactacctgaactggtatcagcagaagcccggcaaggcccccaagctgctgatctactacacctctagactggaaagcggcgtgcccagccggtttagcggcagcggctccggcaccgactacaccctgaccatcagcagcctgcagcccgaggacttcgccacctactactgccagcagggcaacacactcccctggaccttcggccagggcaccaaggtggagatcaagcgtacggtggccgctcccagcgtgttcatcttcccccccagcgacgagcagctgaagtccggcaccgccagcgtcgtgtgcctgctgaacaacttctacccccgggaggccaaggtgcagtggaaggtggacaacgccctgcagagcggcaacagccaggaaagcgtcaccgagcaggacagcaaggactccacctacagcctgagcagcaccctgaccctgagcaaggccgactacgagaagcacaaggtgtacgcctgcgaagtgacccaccagggcctgtccagccccgtgaccaagagcttcaaccggggcgagtgcagcggcggaggctctggaggcggctctgaaggcggaggaagtgagggcggaggctcagaaggcggcggaagcgaaggtggcggctctggcggcggatccggcgaggtgcagctggtcgagtccggcggaggcctggtgcagcctggcggcagcctgagactgagctgcgccgccagcggctacagcttcaccggctacaccatgaactgggtccggcaggctcctggcaagggcctcgaatgggtggccctgatcaacccctacaagggcgtgagcacctacaaccagaagttcaaggaccggttcaccatcagcgtggacaagagcaagaacaccgcctatctgcagatgaacagcctgcgggccgaggacaccgccgtgtactactgcgccagaagcggctactacggcgacagcgactggtacttcgacgtgtggggccagggcacactggtcaccgtgtccagcgctagcaccaagggccctagcgtgttccctctggcccctagcagcaagagcacaagtggaggaacagccgccctgggctgcctggtcaaggactacttccccgagcccgtgaccgtgtcctggaattctggcgccctgacaagcggcgtgcacacatttccagccgtgctgcagagcagcggcctgtactctctgagcagcgtcgtgaccgtgccctctagctctctgggcacccagacctacatctgcaacgtgaaccacaagcccagcaacaccaaagtggacaagaaggtggaacccaagagctgcgacaagacccacacctgtcccccttgccctgcccctgaagctgctggtggcccttccgtgttcctgttccccccaaagcccaaggacaccctgatgatcagccggacccccgaagtgacctgcgtggtggtcgatgtgtcccacgaggaccctgaagtgaagttcaattggtacgtggacggcgtggaagtgcacaatgccaagaccaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtacaccctgcccccatgccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaataa

SEQ ID NO: 45 - protein, 450 aa, synthetic construct
  source 1..450: note = GA201 (VH-CH1)-Fc(hole) P329G LALA
QVQLVQSGAEVKKPGSSVKVSCKASGFTFTDYKIHWVRQAPGQGLEWMGYFNPNSGYSTYAQKFQGRVTITADKSTSTAYMELSSLRSEDTAVYYCARLSPGGYYVMDAWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 46 - DNA, 1353 bp, synthetic construct
  source 1..1353: mol_type = other DNA; note = GA201 (VH-CH1)-Fc(hole) P329G LALA
caggtgcagctggtgcagtctggggctgaggtgaagaagcctgggtcctcggtgaaggtctcctgcaaggcctctggtttcacattcactgactacaagatacactgggtgcgacaggcccctggacaagggctcgagtggatgggatatttcaaccctaacagcggttatagtacctacgcacagaagttccagggcagggtcaccattaccgcggacaaatccacgagcacagcctacatggagctgagcagcctgagatctgaggacacggccgtgtattactgtgcgagactatccccaggcggttactatgttatggatgcctggggccaagggaccaccgtgaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagctgcagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtgcaccctgcccccatcccgggatgagctgaccaagaaccaggtcagcctctcgtgcgcagtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 47 - protein, 213 aa, synthetic construct
  source 1..213: note = GA201 (VL-CL)
DIQMTQSPSSLSASVGDRVTITCRASQGINNYLNWYQQKPGKAPKRLIYNTNNLQTGVPSRFSGSGSGTEFTLTISSLQPEDFATYYCLQHNSFPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 48 - DNA, 642 bp, synthetic construct
  source 1..642: mol_type = other DNA; note = GA201 (VL-CL)
gatatccagatgacccagtctccatcctccctgtctgcatctgtcggagaccgggtcaccatcacctgccgggcaagtcagggcattaacaattacttaaattggtaccagcagaagccagggaaagcccctaagcgcctgatctataataccaacaacttgcagacaggcgtcccatcaaggttcagcggcagtggatccgggacagaattcactctcaccatcagcagcctgcagcctgaagattttgccacctattactgcttgcagcataatagttttcccacgtttggccagggcaccaagctcgagatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 49 - protein, 697 aa, synthetic construct
  source 1..697: note = GA201 (scFab)-Fc(knob) P329G LALA
DIQMTQSPSSLSASVGDRVTITCRASQGINNYLNWYQQKPGKAPKRLIYNTNNLQTGVPSRFSGSGSGTEFTLTISSLQPEDFATYYCLQHNSFPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECSGGGSGGGSEGGGSEGGGSEGGGSEGGGSGGGSGQVQLVQSGAEVKKPGSSVKVSCKASGFTFTDYKIHWVRQAPGQGLEWMGYFNPNSGYSTYAQKFQGRVTITADKSTSTAYMELSSLRSEDTAVYYCARLSPGGYYVMDAWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - DNA, 2094 bp, synthetic construct
  source 1..2094: mol_type = other DNA; note = GA201 (scFab)-Fc(knob) P329G LALA
gatatccagatgacccagtctccatcctccctgtctgcatctgtcggagaccgggtcaccatcacctgccgggcaagtcagggcattaacaattacttaaattggtaccagcagaagccagggaaagcccctaagcgcctgatctataataccaacaacttgcagacaggcgtcccatcaaggttcagcggcagtggatccgggacagaattcactctcaccatcagcagcctgcagcctgaagattttgccacctattactgcttgcagcataatagttttcccacgtttggccagggcaccaagctcgagatcaagcgtacggtggccgctcccagcgtgttcatcttcccccccagcgacgagcagctgaaatctggcaccgccagcgtcgtgtgcctgctgaacaacttctacccccgggaggccaaggtgcagtggaaggtggacaacgccctgcagagcggcaacagccaggaaagcgtcaccgagcaggacagcaaggactccacctatagcctgtccagcaccctgaccctgagcaaggccgactacgagaagcacaaggtgtacgcctgcgaagtgacccaccagggcctgagcagccccgtgaccaagagcttcaaccggggcgagtgcagcggcggaggtagcggaggcggctctgagggcggaggaagcgagggcggaggctccgaaggcggcggaagcgaaggtggcggctctggcggcggatccggccaggtgcagctggtgcagtctggggctgaggtgaagaagcctgggtcctcggtgaaggtctcctgcaaggcctctggtttcacattcactgactacaagatacactgggtgcgacaggcccctggacaagggctcgagtggatgggatatttcaaccctaacagcggttatagtacctacgcacagaagttccagggcagggtcaccattaccgcggacaaatccacgagcacagcctacatggagctgagcagcctgagatctgaggacacggccgtgtattactgtgcgagactatccccaggcggttactatgttatggatgcctggggccaagggaccaccgtgaccgtctcctcagctagcaccaagggccctagcgtgttccctctggcccctagcagcaagagcacaagtggaggaacagccgccctgggctgcctggtcaaggactacttccccgagcccgtgaccgtgtcctggaattctggcgccctgacaagcggcgtgcacacatttccagccgtgctgcagagcagcggcctgtactctctgagcagcgtcgtgaccgtgccctctagctctctgggcacccagacctacatctgcaacgtgaaccacaagcccagcaacaccaaagtggacaagaaggtggaacccaagagctgcgacaagacccacacctgtcccccttgccctgcccctgaagctgctggtggcccttccgtgttcctgttccccccaaagcccaaggacaccctgatgatcagccggacccccgaagtgacctgcgtggtggtcgatgtgtcccacgaggaccctgaagtgaagttcaattggtacgtggacggcgtggaagtgcacaatgccaagaccaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtacaccctgcccccatgccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaataa

SEQ ID NO: 51 - protein, 452 aa, synthetic construct
  source 1..452: note = V9 (VH-CH1)-Fc(hole) P329G LALA
EVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 52 - DNA, 1359 bp, synthetic construct
  source 1..1359: mol_type = other DNA; note = V9 (VH-CH1)-Fc(hole) P329G LALA
gaggtgcagctggtcgagagcggaggcggcctggtgcagcctggcggcagcctgagactgagctgcgccgccagcggctacagcttcaccggctacaccatgaactgggtccggcaggcacctggcaagggactggaatgggtggccctgatcaacccctacaagggcgtgagcacctacaaccagaagttcaaggaccggttcaccatcagcgtggacaagagcaagaacaccgcctatctgcagatgaacagcctgcgggccgaggacaccgccgtgtactactgcgccagaagcggctactacggcgacagcgactggtacttcgacgtgtggggccagggcaccctcgtgaccgtgtctagcgctagcaccaagggcccctccgtgttccccctggcccccagcagcaagagcaccagcggcggcacagccgctctgggctgcctggtcaaggactacttccccgagcccgtgaccgtgtcctggaacagcggagccctgacctccggcgtgcacaccttccccgccgtgctgcagagttctggcctgtatagcctgagcagcgtggtcaccgtgccttctagcagcctgggcacccagacctacatctgcaacgtgaaccacaagcccagcaacaccaaggtggacaagaaggtggagcccaagagctgcgacaaaactcacacatgcccaccgtgcccagcacctgaagctgcagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtgcaccctgcccccatcccgggatgagctgaccaagaaccaggtcagcctctcgtgcgcagtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 53 - protein, 934 aa, synthetic construct [sequence omitted]

SEQ ID NO: 54 - DNA, 2805 bp, synthetic construct
  source 1..2805: mol_type = other DNA; note = V9 (scFab)-GA201 (VH-CH1)-Fc(knob) P329G LALA
gacatccagatgacccagagcccctctagcctgagcgccagcgtgggcgacagagtgaccatcacctgtcgggccagccaggacatcagaaactacctgaactggtatcagcagaagcccggcaaggcccccaagctgctgatctactacacctctagactggaaagcggcgtgcccagccggtttagcggcagcggctccggcaccgactacaccctgaccatcagcagcctgcagcccgaggacttcgccacctactactgccagcagggcaacacactcccctggaccttcggccagggcaccaaggtggagatcaagcgtacggtggccgctcccagcgtgttcatcttcccccccagcgacgagcagctgaagtccggcaccgccagcgtcgtgtgcctgctgaacaacttctacccccgggaggccaaggtgcagtggaaggtggacaacgccctgcagagcggcaacagccaggaaagcgtcaccgagcaggacagcaaggactccacctacagcctgagcagcaccctgaccctgagcaaggccgactacgagaagcacaaggtgtacgcctgcgaagtgacccaccagggcctgtccagccccgtgaccaagagcttcaaccggggcgagtgcagcggcggaggctctggaggcggctctgaaggcggaggaagtgagggcggaggctcagaaggcggcggaagcgaaggtggcggctctggcggcggatccggcgaggtgcagctggtcgagtccggcggaggcctggtgcagcctggcggcagcctgagactgagctgcgccgccagcggctacagcttcaccggctacaccatgaactgggtccggcaggctcctggcaagggcctcgaatgggtggccctgatcaacccctacaagggcgtgagcacctacaaccagaagttcaaggaccggttcaccatcagcgtggacaagagcaagaacaccgcctatctgcagatgaacagcctgcgggccgaggacaccgccgtgtactactgcgccagaagcggctactacggcgacagcgactggtacttcgacgtgtggggccagggcacactggtcaccgtgtccagcgctagcaccaagggcccctccgtgttccccctggcccccagcagcaagagcaccagcggcggcacagccgccctcggctgcctggtcaaggactacttccccgagcccgtgaccgtgtcctggaacagcggagccctgacctccggcgtgcacaccttccccgccgtgctgcagagcagcggcctgtacagcctgtccagcgtggtcaccgtgccctccagcagcctgggcacccagacctacatctgcaacgtgaaccacaagcccagcaataccaaggtggacaagaaggtggagcccaagagctgcgacggcggtggtggctccggaggcggtggatctcaggtgcagctggtgcagtctggggctgaggtgaagaagcctgggtcctcggtgaaggtctcctgcaaggcctctggtttcacattcactgactacaagatacactgggtgcgacaggcccctggacaagggctcgagtggatgggatatttcaaccctaacagcggttatagtacctacgcacagaagttccagggcagggtcaccattaccgcggacaaatccacgagcacagcctacatggagctgagcagcctgagatctgaggacacggccgtgtattactgtgcgagactatccccaggcggttactatgttatggatgcctggggccaagggaccaccgtgaccgtctcctcagctagcaccaagggcccctccgtgttccccctggcccccagcagcaagagcaccagcggcggcacagccgctctgggctgcctggtcaaggactacttccccgagcccgtgaccgtgtcctggaacagcggagccctgacctccggcgtgcacaccttccccgccgtgctgcagagttctggcctgtatagcctgagcagcgtggtcaccgtgccttctagcagcctgggcacccagacctacatctgcaacgtgaaccacaagcccagcaacaccaaggtggacaagaaggtggagcccaagagctgcgacaaaactcacacatgcccaccgtgcccagcacctgaagctgcagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtacaccctgcccccatgccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 55 - protein, 694 aa, synthetic construct
  source 1..694: note = 3F2 (scFab)-Fc(knob) P329G LALA
EIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - DNA, 2085 bp, synthetic construct
  source 1..2085: mol_type = other DNA; note = 3F2 (scFab)-Fc(knob) P329G LALA
gagatcgtgctgacacagagccccggaaccctgtctctgagccctggcgaaagagccaccctgagctgtagagccagccagagcgtgaccagcagctacctggcctggtatcagcagaagcctggacaggcccccagactgctgatcaatgtgggcagcagacgggccaccggcatccctgatagattttctggcagcggcagcggcaccgacttcaccctgaccatcagcagactggaacccgaggacttcgccgtgtactactgccagcagggcatcatgctgccccctacatttggccagggcaccaaggtggaaatcaagcgtacggtggccgctcccagcgtgttcatcttcccacctagcgacgagcagctgaagtctggcacagccagcgtcgtgtgcctgctgaacaacttctacccccgcgaggccaaggtgcagtggaaggtggacaacgccctgcagagcggcaacagccaggaaagcgtcaccgagcaggacagcaaggactccacctacagcctgagcagcaccctgaccctgagcaaggccgactacgagaagcacaaggtgtacgcctgcgaagtgacccaccagggcctgtctagccccgtgaccaagagcttcaaccggggagaatgtggcggcggaggatctggtggcggaggtagtggtggtggtggatctggcggaggcggatccggcggaggtggaagcggaggtggtggaagtgggggagaagtgcagctgctggaaagtggcggaggcctggtgcagcctggcggatctctgagactgagctgtgccgccagcggcttcacctttagcagctacgccatgagctgggtccgacaggcccctggaaagggactggaatgggtgtccgccatctctggctctggcggcagcacctactacgccgatagcgtgaagggccggttcaccatcagccgggacaacagcaagaacaccctgtacctgcagatgaacagcctgcgggccgaggataccgccgtgtattattgcgccaagggatggttcggcggcttcaactattggggccagggaaccctggtcaccgtgtctagtgctagcaccaagggccctagcgtgttccctctggcccctagcagcaagagcacaagtggaggaacagccgccctgggctgcctggtcaaggactacttccccgagcccgtgaccgtgtcctggaattctggcgccctgacaagcggcgtgcacacatttccagccgtgctgcagagcagcggcctgtactctctgagcagcgtcgtgaccgtgccctctagctctctgggcacccagacctacatctgcaacgtgaaccacaagcccagcaacaccaaagtggacaagaaggtggaacccaagagctgcgacaagacccacacctgtcccccttgccctgcccctgaagctgctggtggcccttccgtgttcctgttccccccaaagcccaaggacaccctgatgatcagccggacccccgaagtgacctgcgtggtggtcgatgtgtcccacgaggaccctgaagtgaagttcaattggtacgtggacggcgtggaagtgcacaatgccaagaccaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtacaccctgcccccatgccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaataa

SEQ ID NO: 57 - protein, 931 aa, synthetic construct
  source 1..931: note = V9 (scFab)-3F2 (VH-CH1)-Fc(knob) P329G LALA
DIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECSGGGSGGGSEGGGSEGGGSEGGGSEGGGSGGGSGEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 58 - DNA, 2796 bp, synthetic construct
  source 1..2796: mol_type = other DNA; note = V9 (scFab)-3F2 (VH-CH1)-Fc(knob) P329G LALA
gacatccagatgacccagagcccctctagcctgagcgccagcgtgggcgacagagtgaccatcacctgtcgggccagccaggacatcagaaactacctgaactggtatcagcagaagcccggcaaggcccccaagctgctgatctactacacctctagactggaaagcggcgtgcccagccggtttagcggcagcggctccggcaccgactacaccctgaccatcagcagcctgcagcccgaggacttcgccacctactactgccagcagggcaacacactcccctggaccttcggccagggcaccaaggtggagatcaagcgtacggtggccgctcccagcgtgttcatcttcccccccagcgacgagcagctgaagtccggcaccgccagcgtcgtgtgcctgctgaacaacttctacccccgggaggccaaggtgcagtggaaggtggacaacgccctgcagagcggcaacagccaggaaagcgtcaccgagcaggacagcaaggactccacctacagcctgagcagcaccctgaccctgagcaaggccgactacgagaagcacaaggtgtacgcctgcgaagtgacccaccagggcctgtccagccccgtgaccaagagcttcaaccggggcgagtgcagcggcggaggctctggaggcggctctgaaggcggaggaagtgagggcggaggctcagaaggcggcggaagcgaaggtggcggctctggcggcggatccggcgaggtgcagctggtcgagtccggcggaggcctggtgcagcctggcggcagcctgagactgagctgcgccgccagcggctacagcttcaccggctacaccatgaactgggtccggcaggctcctggcaagggcctcgaatgggtggccctgatcaacccctacaagggcgtgagcacctacaaccagaagttcaaggaccggttcaccatcagcgtggacaagagcaagaacaccgcctatctgcagatgaacagcctgcgggccgaggacaccgccgtgtactactgcgccagaagcggctactacggcgacagcgactggtacttcgacgtgtggggccagggcacactggtcaccgtgtccagcgctagcaccaagggcccctccgtgttccccctggcccccagcagcaagagcaccagcggcggcacagccgccctcggctgcctggtcaaggactacttccccgagcccgtgaccgtgtcctggaacagcggagccctgacctccggcgtgcacaccttccccgccgtgctgcagagcagcggcctgtacagcctgtccagcgtggtcaccgtgccctccagcagcctgggcacccagacctacatctgcaacgtgaaccacaagcccagcaataccaaggtggacaagaaggtggagcccaagagctgcgacggcggtggtggctccggaggaggaggcagcgaggtgcagctgctggaatctggaggcggcctggtgcagcctggcggcagcctgagactgtcttgcgccgccagcggcttcaccttcagcagctacgccatgagctgggtccgacaggctcctggcaagggactggaatgggtgtccgccatctccggcagcggaggcagcacctactacgccgacagcgtgaagggccggttcaccatcagcagagacaacagcaagaacaccctgtacctgcagatgaacagcctgcgggccgaggataccgccgtgtattattgcgccaagggatggttcggcggcttcaactactggggccagggaaccctggtgacagtgtccagcgccagcaccaagggcccctccgtgtttcctctggcccccagcagcaagagcacctctggcggaacagccgccctgggctgcctggtgaaagactacttccccgagcccgtgaccgtgtcctggaactctggcgccctgaccagcggcgtgcacacctttccagccgtgctgcagagcagcggcctgtactccctgagcagcgtggtgacagtgccctccagcagcctgggcacccagacctacatctgcaacgtgaaccacaagcccagcaacaccaaagtggacaagaaggtggaacccaagagctgcgacaaaactcacacatgcccaccgtgcccagcacctgaagctgcagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtacaccctgcccccatgccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 59 - protein, 447 aa, synthetic construct
  source 1..447: note = 3F2 (VH-CH1)-Fc(hole) P329G LALA
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 60 - DNA, 1344 bp, synthetic construct
  source 1..1344: mol_type = other DNA; note = 3F2 (VH-CH1)-Fc(hole) P329G LALA
gaggtgcagctgctggaatctggaggcggcctggtgcagcctggcggcagcctgagactgtcttgcgccgccagcggcttcaccttcagcagctacgccatgagctgggtccgacaggctcctggcaagggactggaatgggtgtccgccatctccggcagcggaggcagcacctactacgccgacagcgtgaagggccggttcaccatcagcagagacaacagcaagaacaccctgtacctgcagatgaacagcctgcgggccgaggataccgccgtgtattattgcgccaagggatggttcggcggcttcaactactggggccagggaaccctggtgacagtgtccagcgccagcaccaagggcccctccgtgtttcctctggcccccagcagcaagagcacctctggcggaacagccgccctgggctgcctggtgaaagactacttccccgagcccgtgaccgtgtcctggaactctggcgccctgaccagcggcgtgcacacctttccagccgtgctgcagagcagcggcctgtactccctgagcagcgtggtgacagtgccctccagcagcctgggcacccagacctacatctgcaacgtgaaccacaagcccagcaacaccaaagtggacaagaaggtggaacccaagagctgcgacaaaactcacacatgcccaccgtgcccagcacctgaagctgcagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtgcaccctgcccccatcccgggatgagctgaccaagaaccaggtcagcctctcgtgcgcagtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 61 - protein, 215 aa, synthetic construct
  source 1..215: note = 3F2 (VL-CL)
EIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 62 - DNA, 648 bp, synthetic construct
  source 1..648: mol_type = other DNA; note = 3F2 (VL-CL)
gagatcgtgctgacccagtctcccggcaccctgagcctgagccctggcgagagagccaccctgagctgcagagccagccagagcgtgaccagcagctacctggcctggtatcagcagaagcccggccaggcccccagactgctgatcaacgtgggcagcagacgggccaccggcatccccgatagattcagcggcagcggctccggcaccgacttcaccctgaccatcagccggctggaacccgaggacttcgccgtgtactactgccagcagggcatcatgctgccccccaccttcggccagggcaccaaggtggaaatcaagcggaccgtggccgctcccagcgtgttcatcttcccacccagcgacgagcagctgaagtccggcacagccagcgtggtgtgcctgctgaacaacttctacccccgcgaggccaaggtgcagtggaaggtggacaacgccctgcagagcggcaacagccaggaatccgtgaccgagcaggacagcaaggactccacctacagcctgagcagcaccctgaccctgagcaaggccgactacgagaagcacaaggtgtacgcctgcgaagtgacccaccagggcctgtccagccccgtgaccaagagcttcaaccggggcgagtgctga

SEQ ID NO: 63 - protein, 673 aa, synthetic construct
  source 1..673: note = CH1A1A (VH-CH1)- V9 (VL-CH1)-Fc(knob) P329G LALA
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSDIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 64 - DNA, 2022 bp, synthetic construct
  source 1..2022: mol_type = other DNA; note = CH1A1A (VH-CH1)- V9 (VL-CH1)-Fc(knob) P329G LALA
caggtgcagctggtgcagtctggcgccgaagtgaagaaacctggcgccagcgtgaaggtgtcctgcaaggccagcggctacaccttcaccgagttcggcatgaactgggtccgacaggcccctggacagggcctggaatggatgggctggatcaacaccaagaccggcgaggccacctacgtggaagagttcaagggcagagtgaccttcaccaccgacaccagcaccagcaccgcctacatggaactgcggagcctgagaagcgacgacaccgccgtgtactactgcgccagatgggacttcgcctactatgtggaagccatggactactggggccagggcaccaccgtgaccgtgtctagtgctagcacaaagggccccagcgtgttccctctggcccctagcagcaagagcacatctggcggaacagccgccctgggctgcctggtcaaggactactttcccgagcccgtgacagtgtcctggaactctggcgccctgacaagcggcgtgcacacctttccagccgtgctgcagagcagcggcctgtactctctgagcagcgtggtcaccgtgcctagctctagcctgggcacccagacctacatctgcaacgtgaaccacaagcccagcaacaccaaggtggacaagaaggtggaacccaagagctgcggcggaggcggatccggaggcggaggatctgatatccagatgacccagagccccagcagcctgtctgccagcgtgggcgacagagtgaccattacctgcagagccagccaggacatcagaaactacctgaactggtatcagcagaagcccggcaaggcccccaagctgctgatctactacaccagcagactggaatccggcgtgcccagcagattttccggcagcggctctggcaccgactacaccctgacaatcagcagcctgcagcccgaggacttcgccacctactactgccagcagggcaacaccctgccctggacatttggacagggcacaaaggtggaaatcaagagcagcgcctccaccaagggcccttccgtgtttccactggcccccagctctaagagcaccagcggaggaacagctgctctgggatgtctcgtgaaggattacttccccgaacctgtgaccgtcagctggaacagcggcgctctgacatctggggtgcacacattccccgctgtcctgcagtcctccggcctgtacagtctgtccagcgtcgtgacagtgcctagcagctccctgggaacacagacatatatctgtaatgtcaatcacaagccctctaataccaaggtcgacaaaaaagtcgagcccaagtcctgcgacaagacccacacctgtcccccttgtcctgcccctgaagctgctggcggcccttctgtgttcctgttccccccaaagcccaaggacaccctgatgatcagccggacccccgaagtgacctgcgtggtggtggatgtgtcccacgaggaccctgaagtgaagttcaattggtacgtggacggcgtggaagtgcacaacgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtacaccctgcccccatgccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 65 - protein, 451 aa, synthetic construct
  source 1..451: note = CH1A1A (VH-CH1)-Fc(hole) P329G LALA
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 66 - DNA, 1356 bp, synthetic construct
  source 1..1356: mol_type = other DNA; note = CH1A1A (VH-CH1)-Fc(hole) P329G LALA
caggtgcagctggtgcagtctggcgccgaagtgaagaaacctggagctagtgtgaaggtgtcctgcaaggccagcggctacaccttcaccgagttcggcatgaactgggtccgacaggctccaggccagggcctcgaatggatgggctggatcaacaccaagaccggcgaggccacctacgtggaagagttcaagggcagagtgaccttcaccacggacaccagcaccagcaccgcctacatggaactgcggagcctgagaagcgacgacaccgccgtgtactactgcgccagatgggacttcgcctattacgtggaagccatggactactggggccagggcaccaccgtgaccgtgtctagcgctagcaccaagggcccctccgtgttccccctggcccccagcagcaagagcaccagcggcggcacagccgctctgggctgcctggtcaaggactacttccccgagcccgtgaccgtgtcctggaacagcggagccctgacctccggcgtgcacaccttccccgccgtgctgcagagttctggcctgtatagcctgagcagcgtggtcaccgtgccttctagcagcctgggcacccagacctacatctgcaacgtgaaccacaagcccagcaacaccaaggtggacaagaaggtggagcccaagagctgcgacaaaactcacacatgcccaccgtgcccagcacctgaagctgcagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtgcaccctgcccccatcccgggatgagctgaccaagaaccaggtcagcctctcgtgcgcagtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 67 - protein, 215 aa, synthetic construct
  source 1..215: note = CH1A1A (VL-CL)
DIQMTQSPSSLSASVGDRVTITCKASAAVGTYVAWYQQKPGKAPKLLIYSASYRKRGVPSRFSGSGSGTDFTLTISSLQPEDFATYYCHQYYTYPLFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 68 - DNA, 648 bp, synthetic construct
  source 1..648: mol_type = other DNA; note = CH1A1A (VL-CL)
gatatccagatgacccagtctccatcctccctgtctgcatctgtgggagacagagtcaccatcacttgcaaggccagtgcggctgtgggtacgtatgttgcgtggtatcagcagaaaccagggaaagcacctaagctcctgatctattcggcatcctaccgcaaaaggggagtcccatcaaggttcagtggcagtggatctgggacagatttcactctcaccatcagcagtctgcaacctgaagatttcgcaacttactactgtcaccaatattacacctatcctctattcacgtttggccagggcaccaagctcgagatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 69 - protein, 11 aa, synthetic construct
  source 1..11: note = LC007 HCDR1
GYSITSGYYWN

SEQ ID NO: 70 - DNA, 33 bp, synthetic construct
  source 1..33: mol_type = other DNA; note = LC007 HCDR1
ggctactccatcaccagtggttattactggaac

SEQ ID NO: 71 - protein, 16 aa, synthetic construct
  source 1..16: note = LC007 HCDR2
YITYDGSNNYNPSLKN

SEQ ID NO: 72 - DNA, 48 bp, synthetic construct
  source 1..48: mol_type = other DNA; note = LC007 HCDR2
tacataacctacgacggtagcaataactacaacccatctctcaaaaat

SEQ ID NO: 73-74 - skipped (2 entries)

SEQ ID NO: 75 - protein, 112 aa, synthetic construct
  source 1..112: note = LC007 VH
EVQLQESGPGLVKPSQSLSLTCSVTGYSITSGYYWNWIRQFPGNKLEWMGYITYDGSNNYNPSLKNRISITRDTSKNQFFLKLNSVTTEDTATYYCADFDYWGQGTTLTVSS

SEQ ID NO: 76 - DNA, 336 bp, synthetic construct
  source 1..336: mol_type = other DNA; note = LC007 VH
gaggtccagctgcaggagtcaggacctggcctcgtgaaaccttctcagtctctgtctctcacctgctctgtcactggctactccatcaccagtggttattactggaactggatccggcagtttccaggaaacaagctggaatggatgggctacataacctacgacggtagcaataactacaacccatctctcaaaaatcgaatctccatcactcgtgacacatctaagaaccagtttttcctgaagttgaattctgtgactactgaggacacagctacatattactgtgcggactttgactactggggccaaggcaccactctcacagtctcctca

SEQ ID NO: 77 - protein, 11 aa, synthetic construct
  source 1..11: note = LC007 LCDR1
SASQGIRNYLN

SEQ ID NO: 78 - DNA, 33 bp, synthetic construct
  source 1..33: mol_type = other DNA; note = LC007 LCDR1
agtgcaagtcagggcattagaaattatttaaac

SEQ ID NO: 79 - protein, 7 aa, synthetic construct
  source 1..7: note = LC007 LCDR2
YTSSLHS

SEQ ID NO: 80 - DNA, 21 bp, synthetic construct
  source 1..21: mol_type = other DNA; note = LC007 LCDR2
tacacatcaagtttacactca

SEQ ID NO: 81 - protein, 9 aa, synthetic construct
  source 1..9: note = LC007 LCDR3
QQYSKLPWT

SEQ ID NO: 82 - DNA, 27 bp, synthetic construct
  source 1..27: mol_type = other DNA; note = LC007 LCDR3
cagcagtatagtaagcttccttggacg

SEQ ID NO: 83 - protein, 107 aa, synthetic construct
  source 1..107: note = LC007 VL
DIVLTQSPSSLSASLGDRVTISCSASQGIRNYLNWYQQRPDGTVKLLIYYTSSLHSGVPSRFSGSGSGTDYSLTISNLEPEDIATYYCQQYSKLPWTFGGGTKLEIK

SEQ ID NO: 84 - DNA, 321 bp, synthetic construct
  source 1..321: mol_type = other DNA; note = LC007 VL
gatattgtgctcacacagtctccatcctccctgtctgcctctctgggagacagagtcaccatcagttgcagtgcaagtcagggcattagaaattatttaaactggtatcagcagagaccagatggaactgttaaactcctgatctattacacatcaagtttacactcaggagtcccatcaaggttcagtggcagtgggtctgggacagattattctctcaccatcagcaacctggaacctgaagatattgccacttactattgtcagcagtatagtaagcttccttggacgttcggtggaggcaccaagctggaaatcaaa

SEQ ID NO: 85 - protein, 5 aa, synthetic construct
  source 1..5: note = GA201 HCDR1
DYKIH

SEQ ID NO: 86 - DNA, 15 bp, synthetic construct
  source 1..15: mol_type = other DNA; note = GA201 HCDR1
gactacaagatacac

SEQ ID NO: 87 - protein, 17 aa, synthetic construct
  source 1..17: note = GA201 HCDR2
YFNPNSGYSTYAQKFQG

SEQ ID NO: 88 - DNA, 51 bp, synthetic construct
  source 1..51: mol_type = other DNA; note = GA201 HCDR2
tatttcaaccctaacagcggttatagtacctacgcacagaagttccagggc

SEQ ID NO: 89 - protein, 11 aa, synthetic construct
  source 1..11: note = GA201 HCDR3
LSPGGYYVMDA

SEQ ID NO: 90 - DNA, 33 bp, synthetic construct
  source 1..33: mol_type = other DNA; note = GA201 HCDR3
ctatccccaggcggttactatgttatggatgcc

SEQ ID NO: 91 - protein, 120 aa, synthetic construct [sequence omitted]

SEQ ID NO: 92 - DNA, 360 bp, synthetic construct
  source 1..360: mol_type = other DNA; note = GA201 VH
caggtgcagctggtgcagtctggggctgaggtgaagaagcctgggtcctcggtgaaggtctcctgcaaggcctctggtttcacattcactgactacaagatacactgggtgcgacaggcccctggacaagggctcgagtggatgggatatttcaaccctaacagcggttatagtacctacgcacagaagttccagggcagggtcaccattaccgcggacaaatccacgagcacagcctacatggagctgagcagcctgagatctgaggacacggccgtgtattactgtgcgagactatccccaggcggttactatgttatggatgcctggggccaagggaccaccgtgaccgtctcctca

SEQ ID NO: 93 - protein, 11 aa, synthetic construct
  source 1..11: note = GA201 LCDR1
RASQGINNYLN

SEQ ID NO: 94 - DNA, 33 bp, synthetic construct
  source 1..33: mol_type = other DNA; note = GA201 LCDR1
cgggcaagtcagggcattaacaattacttaaat

SEQ ID NO: 95 - protein, 7 aa, synthetic construct
  source 1..7: note = GA201 LCDR2
NTNNLQT

SEQ ID NO: 96 - DNA, 21 bp, synthetic construct
  source 1..21: mol_type = other DNA; note = GA201 LCDR2
aataccaacaacttgcagaca

SEQ ID NO: 97 - protein, 8 aa, synthetic construct
  source 1..8: note = GA201 LCDR3
LQHNSFPT

SEQ ID NO: 98 - DNA, 24 bp, synthetic construct
  source 1..24: mol_type = other DNA; note = GA201 LCDR3
ttgcagcataatagttttcccacg

SEQ ID NO: 99 - protein, 106 aa, synthetic construct [sequence omitted]

SEQ ID NO: 100 - DNA, 318 bp, synthetic construct
  source 1..318: mol_type = other DNA; note = GA201 VL
gatatccagatgacccagtctccatcctccctgtctgcatctgtcggagaccgggtcaccatcacctgccgggcaagtcagggcattaacaattacttaaattggtaccagcagaagccagggaaagcccctaagcgcctgatctataataccaacaacttgcagacaggcgtcccatcaaggttcagcggcagtggatccgggacagaattcactctcaccatcagcagcctgcagcctgaagattttgccacctattactgcttgcagcataatagttttcccacgtttggccagggcaccaagctcgagatcaag

SEQ ID NO: 101 - protein, 5 aa, synthetic construct
  source 1..5: note = 3F2 HCDR1
SYAMS

SEQ ID NO: 102 - DNA, 15 bp, synthetic construct
  source 1..15: mol_type = other DNA; note = 3F2 HCDR1
agctacgccatgagc

SEQ ID NO: 103 - protein, 16 aa, synthetic construct
  source 1..16: note = 3F2 HCDR2
AISGSGGSTYYADSVK

SEQ ID NO: 104 - DNA, 48 bp, synthetic construct
  source 1..48: mol_type = other DNA; note = 3F2 HCDR2
gccatctccggcagcggaggcagcacctactacgccgacagcgtgaag

SEQ ID NO: 105 - protein, 8 aa, synthetic construct
  source 1..8: note = 3F2 HCDR3
YCAKGWFG

SEQ ID NO: 106 - DNA, 24 bp, synthetic construct
  source 1..24: mol_type = other DNA; note = 3F2 HCDR3
tattgcgccaagggatggttcggc

SEQ ID NO: 107 - protein, 117 aa, synthetic construct
  source 1..117: note = 3F2 VH
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGWFGGFNYWGQGTLVTVSS

SEQ ID NO: 108 - DNA, 351 bp, synthetic construct
  source 1..351: mol_type = other DNA; note = 3F2 VH
gaggtgcagctgctggaatctggaggcggcctggtgcagcctggcggcagcctgagactgtcttgcgccgccagcggcttcaccttcagcagctacgccatgagctgggtccgacaggctcctggcaagggactggaatgggtgtccgccatctccggcagcggaggcagcacctactacgccgacagcgtgaagggccggttcaccatcagcagagacaacagcaagaacaccctgtacctgcagatgaacagcctgcgggccgaggataccgccgtgtattattgcgccaagggatggttcggcggcttcaactactggggccagggaaccctggtgacagtgtccagc

SEQ ID NO: 109 - protein, 11 aa, synthetic construct
  source 1..11: note = 3F2 LCDR1
RASQSVTSSYL

SEQ ID NO: 110 - DNA, 33 bp, synthetic construct
  source 1..33: mol_type = other DNA; note = 3F2 LCDR1
agagccagccagagcgtgaccagcagctacctg

SEQ ID NO: 111 - protein, 7 aa, synthetic construct
  source 1..7: note = 3F2 LCDR2
NVGSRRA

SEQ ID NO: 112 - DNA, 21 bp, synthetic construct
  source 1..21: mol_type = other DNA; note = 3F2 LCDR2
aacgtgggcagcagacgggcc

SEQ ID NO: 113 - protein, 9 aa, synthetic construct
  source 1..9: note = 3F2 LCDR3
CQQGIMLPP

SEQ ID NO: 114 - DNA, 27 bp, synthetic construct
  source 1..27: mol_type = other DNA; note = 3F2 LCDR3
tgccagcagggcatcatgctgcccccc

SEQ ID NO: 115 - protein, 108 aa, synthetic construct
  source 1..108: note = 3F2 VL
EIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIK

SEQ ID NO: 116 - DNA, 324 bp, synthetic construct
  source 1..324: mol_type = other DNA; note = 3F2 VL
gagatcgtgctgacccagtctcccggcaccctgagcctgagccctggcgagagagccaccctgagctgcagagccagccagagcgtgaccagcagctacctggcctggtatcagcagaagcccggccaggcccccagactgctgatcaacgtgggcagcagacgggccaccggcatccccgatagattcagcggcagcggctccggcaccgacttcaccctgaccatcagccggctggaacccgaggacttcgccgtgtactactgccagcagggcatcatgctgccccccaccttcggccagggcaccaaggtggaaatcaag

SEQ ID NO: 117 - protein, 5 aa, synthetic construct
  source 1..5: note = CH1A1A HCDR1
EFGMN

SEQ ID NO: 118 - DNA, 15 bp, synthetic construct
  source 1..15: mol_type = other DNA; note = CH1A1A HCDR1
gagttcggcatgaac

SEQ ID NO: 119 - protein, 17 aa, synthetic construct
  source 1..17: note = CH1A1A HCDR2
WINTKTGEATYVEEFKG

SEQ ID NO: 120 - DNA, 51 bp, synthetic construct
  source 1..51: mol_type = other DNA; note = CH1A1A HCDR2
tggatcaacaccaagaccggcgaggccacctacgtggaagagttcaagggc

SEQ ID NO: 121 - protein, 12 aa, synthetic construct
  source 1..12: note = CH1A1A HCDR3
WDFAYYVEAMDY

SEQ ID NO: 122 - DNA, 36 bp, synthetic construct
  source 1..36: mol_type = other DNA; note = CH1A1A HCDR3
tgggacttcgcctattacgtggaagccatggactac

SEQ ID NO: 123 - protein, 121 aa, synthetic construct
  source 1..121: note = CH1A1A VH
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSS

SEQ ID NO: 124 - DNA, 363 bp, synthetic construct
  source 1..363: mol_type = other DNA; note = CH1A1A VH
caggtgcagctggtgcagtctggcgccgaagtgaagaaacctggagctagtgtgaaggtgtcctgcaaggccagcggctacaccttcaccgagttcggcatgaactgggtccgacaggctccaggccagggcctcgaatggatgggctggatcaacaccaagaccggcgaggccacctacgtggaagagttcaagggcagagtgaccttcaccacggacaccagcaccagcaccgcctacatggaactgcggagcctgagaagcgacgacaccgccgtgtactactgcgccagatgggacttcgcctattacgtggaagccatggactactggggccagggcaccaccgtgaccgtgtctagc

SEQ ID NO: 125 - protein, 11 aa, synthetic construct
  source 1..11: note = CH1A1A LCDR1
KASAAVGTYVA

SEQ ID NO: 126 - DNA, 33 bp, synthetic construct
  source 1..33: mol_type = other DNA; note = CH1A1A LCDR1
aaggccagtgcggctgtgggtacgtatgttgcg

SEQ ID NO: 127 - protein, 7 aa, synthetic construct
  source 1..7: note = CH1A1A LCDR2
SASYRKR

SEQ ID NO: 128 - DNA, 21 bp, synthetic construct
  source 1..21: mol_type = other DNA; note = CH1A1A LCDR2
tcggcatcctaccgcaaaagg

SEQ ID NO: 129 - protein, 10 aa, synthetic construct
  source 1..10: note = CH1A1A LCDR3
HQYYTYPLFT

SEQ ID NO: 130 - DNA, 30 bp, synthetic construct
  source 1..30: mol_type = other DNA; note = CH1A1A LCDR3
caccaatattacacctatcctctattcacg

SEQ ID NO: 131 - protein, 108 aa, synthetic construct
  source 1..108: note = CH1A1A VL
DIQMTQSPSSLSASVGDRVTITCKASAAVGTYVAWYQQKPGKAPKLLIYSASYRKRGVPSRFSGSGSGTDFTLTISSLQPEDFATYYCHQYYTYPLFTFGQGTKLEIK

SEQ ID NO: 132 - DNA, 324 bp, synthetic construct
  source 1..324: mol_type = other DNA; note = CH1A1A VL
gatatccagatgacccagtctccatcctccctgtctgcatctgtgggagacagagtcaccatcacttgcaaggccagtgcggctgtgggtacgtatgttgcgtggtatcagcagaaaccagggaaagcacctaagctcctgatctattcggcatcctaccgcaaaaggggagtcccatcaaggttcagtggcagtggatctgggacagatttcactctcaccatcagcagtctgcaacctgaagatttcgcaacttactactgtcaccaatattacacctatcctctattcacgtttggccagggcaccaagctcgagatcaag

SEQ ID NO: 133 - protein, 10 aa, synthetic construct
  source 1..10: note = Anti-CD33 HCDR1
GYTITDSNIH

SEQ ID NO: 134 - DNA, 30 bp, synthetic construct
  source 1..30: mol_type = other DNA; note = Anti-CD33 HCDR1
ggctacaccatcaccgacagcaacatccac

SEQ ID NO: 135 - protein, 13 aa, synthetic construct
  source 1..13: note = Anti-CD33 HCDR2
YIYPYNGGTDYNQ

SEQ ID NO: 136 - DNA, 39 bp, synthetic construct
  source 1..39: mol_type = other DNA; note = Anti-CD33 HCDR2
tacatctacccctacaacggcggcaccgactacaaccag

SEQ ID NO: 137 - protein, 7 aa, synthetic construct
  source 1..7: note = Anti-CD33 HCDR3
GNPWLAY

SEQ ID NO: 138 - DNA, 21 bp, synthetic construct
  source 1..21: mol_type = other DNA; note = Anti-CD33 HCDR3
ggcaacccctggctggcctat

SEQ ID NO: 139 - protein, 116 aa, synthetic construct [sequence omitted]

SEQ ID NO: 140 - DNA, 348 bp, synthetic construct
  source 1..348: mol_type = other DNA; note = Anti-CD33 VH
gaagtgcagctggtgcagtctggcgccgaagtgaagaaacccggcagcagcgtgaaggtgtcctgcaaggccagcggctacaccatcaccgacagcaacatccactgggtccgacaggcccctgggcagagcctggaatggatcggctacatctacccctacaacggcggcaccgactacaaccagaagttcaagaaccgggccaccctgaccgtggacaaccccaccaacaccgcctacatggaactgagcagcctgcggagcgaggacaccgccttctactactgcgtgaacggcaacccctggctggcctattggggccagggaaccctggtcaccgtgtctagc

SEQ ID NO: 141 - protein, 15 aa, synthetic construct
  source 1..15: note = Anti-CD33 LCDR1
RASESLDNYGIRFLT

SEQ ID NO: 142 - DNA, 45 bp, synthetic construct
  source 1..45: mol_type = other DNA; note = Anti-CD33 LCDR1
cgggccagcgagagcctggacaactacggcatccggtttctgacc

SEQ ID NO: 143 - protein, 7 aa, synthetic construct
  source 1..7: note = Anti-CD33 LCDR2
AASNQGS

SEQ ID NO: 144 - DNA, 21 bp, synthetic construct
  source 1..21: mol_type = other DNA; note = Anti-CD33 LCDR2
gccgccagcaaccagggcagc

SEQ ID NO: 145 - protein, 9 aa, synthetic construct
  source 1..9: note = Anti-CD33 LCDR3
QQTKEVPWS

SEQ ID NO: 146 - DNA, 27 bp, synthetic construct
  source 1..27: mol_type = other DNA; note = Anti-CD33 LCDR3
cagcagaccaaagaggtgccctggtcc

SEQ ID NO: 147 - protein, 111 aa, synthetic construct [sequence omitted]

SEQ ID NO: 148 - DNA, 333 bp, synthetic construct
  source 1..333: mol_type = other DNA; note = Anti-CD33 VL
gacatccagctgacccagagccccagcaccctgtctgccagcgtgggcgacagagtgaccatcacctgtcgggccagcgagagcctggacaactacggcatccggtttctgacctggttccagcagaagcccggcaaggcccccaagctgctgatgtacgccgccagcaaccagggcagcggcgtgccaagcagattcagcggcagcggctccggcaccgagttcaccctgaccatcagcagcctgcagcccgacgacttcgccacctactactgccagcagaccaaagaggtgccctggtccttcggccagggcaccaaggtggaagtgaag

SEQ ID NO: 149 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 150 - protein, 15 aa, synthetic construct
  source 1..15: note = Linker
EPKSCGGGGSGGGGS

SEQ ID NO: 151 - protein, 16 aa, synthetic construct
  source 1..16: note = Linker
EPKSCDGGGGSGGGGS

SEQ ID NO: 152 - protein, 32 aa, synthetic construct
  source 1..32: note = Linker
GGGGSGGGGSGGGGSGGGGSGGGGSGGGGSGG

SEQ ID NO: 153 - protein, 34 aa, synthetic construct
  source 1..34: note = Linker
SGGGSGGGSEGGGSEGGGSEGGGSEGGGSGGGSG

SEQ ID NO: 154 - protein, 19 aa, synthetic construct
  source 1..19: note = Leader 1
MDWTWRILFLVAAATGAHS

SEQ ID NO: 155 - DNA, 57 bp, synthetic construct
  source 1..57: mol_type = other DNA; note = Leader 1
atggactggacctggagaatcctcttcttggtggcagcagccacaggagcccactcc

SEQ ID NO: 156 - DNA, 57 bp, synthetic construct
  source 1..57: mol_type = other DNA; note = Leader 1
atggactggacctggaggatcctcttcttggtggcagcagccacaggagcccactcc

SEQ ID NO: 157 - protein, 22 aa, synthetic construct
  source 1..22: note = Leader 2
MDMRVPAQLLGLLLLWFPGARC

SEQ ID NO: 158 - DNA, 66 bp, synthetic construct
  source 1..66: mol_type = other DNA; note = Leader 2
atggacatgagggtccccgctcagctcctgggcctcctgctgctctggttcccaggtgccaggtgt

SEQ ID NO: 159 - protein, 19 aa, synthetic construct
  source 1..19: note = Leader 3
MGWSCIILFLVATATGVHS

SEQ ID NO: 160 - DNA, 57 bp, synthetic construct
  source 1..57: mol_type = other DNA; note = Leader 3
atgggatggagctgtatcatcctcttcttggtagcaacagctaccggtgtgcattcc

SEQ ID NO: 161 - DNA, 57 bp, synthetic construct
  source 1..57: mol_type = other DNA; note = Leader 3
atgggctggtcctgcatcatcctgtttctggtggctaccgccactggagtgcattcc

SEQ ID NO: 162 - DNA, 57 bp, synthetic construct
  source 1..57: mol_type = other DNA; note = Leader 3
atgggctggtcctgcatcatcctgtttctggtcgccacagccaccggcgtgcactct

SEQ ID NO: 163 - protein, 5 aa, synthetic construct
  source 1..5: note = V9 HCDR1
GYTMN

SEQ ID NO: 164 - DNA, 15 bp, synthetic construct
  source 1..15: mol_type = other DNA; note = V9 HCDR1
ggctacaccatgaac

SEQ ID NO: 165 - protein, 17 aa, synthetic construct
  source 1..17: note = V9 HCDR2
LINPYKGVSTYNQKFKD

SEQ ID NO: 166 - DNA, 51 bp, synthetic construct
  source 1..51: mol_type = other DNA; note = V9 HCDR2
ctgatcaacccctacaagggcgtgagcacctacaaccagaagttcaaggac

SEQ ID NO: 167 - protein, 13 aa, synthetic construct
  source 1..13: note = V9 HCDR3
SGYYGDSDWYFDV

SEQ ID NO: 168 - DNA, 39 bp, synthetic construct
  source 1..39: mol_type = other DNA; note = V9 HCDR3
agcggctactacggcgacagcgactggtacttcgacgtg

SEQ ID NO: 169 - protein, 122 aa, synthetic construct [sequence omitted]

SEQ ID NO: 170 - DNA, 366 bp, synthetic construct
  source 1..366: mol_type = other DNA; note = V9 VH
gaggtgcagctggtcgagtccggcggaggcctggtgcagcctggcggcagcctgagactgagctgcgccgccagcggctacagcttcaccggctacaccatgaactgggtccggcaggctcctggcaagggcctcgaatgggtggccctgatcaacccctacaagggcgtgagcacctacaaccagaagttcaaggaccggttcaccatcagcgtggacaagagcaagaacaccgcctatctgcagatgaacagcctgcgggccgaggacaccgccgtgtactactgcgccagaagcggctactacggcgacagcgactggtacttcgacgtgtggggccagggcacactggtcaccgtgtccagc

SEQ ID NO: 171 - protein, 11 aa, synthetic construct
  source 1..11: note = V9 LCDR1
RASQDIRNYLN

SEQ ID NO: 172 - DNA, 33 bp, synthetic construct
  source 1..33: mol_type = other DNA; note = V9 LCDR1
cgggccagccaggacatcagaaactacctgaac

SEQ ID NO: 173 - protein, 7 aa, synthetic construct
  source 1..7: note = V9 LCDR2
YTSRLES

SEQ ID NO: 174 - DNA, 21 bp, synthetic construct
  source 1..21: mol_type = other DNA; note = V9 LCDR2
tacacctctagactggaaagc

SEQ ID NO: 175 - protein, 9 aa, synthetic construct
  source 1..9: note = V9 LCDR3
QQGNTLPWT

SEQ ID NO: 176 - DNA, 27 bp, synthetic construct
  source 1..27: mol_type = other DNA; note = V9 LCDR3
cagcagggcaacacactcccctggacc

SEQ ID NO: 177 - protein, 107 aa, synthetic construct [sequence omitted]

SEQ ID NO: 178 - DNA, 321 bp, synthetic construct
  source 1..321: mol_type = other DNA; note = V9 VL
gacatccagatgacccagagcccctctagcctgagcgccagcgtgggcgacagagtgaccatcacctgtcgggccagccaggacatcagaaactacctgaactggtatcagcagaagcccggcaaggcccccaagctgctgatctactacacctctagactggaaagcggcgtgcccagccggtttagcggcagcggctccggcaccgactacaccctgaccatcagcagcctgcagcccgaggacttcgccacctactactgccagcagggcaacacactcccctggaccttcggccagggcaccaaggtggagatcaag

SEQ ID NO: 179 - protein, 454 aa, synthetic construct
  source 1..454: note = V9(VH-CL)-LC007(VL-CL)
EVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDGGGGSGGGGSDIVLTQSPSSLSASLGDRVTISCSASQGIRNYLNWYQQRPDGTVKLLIYYTSSLHSGVPSRFSGSGSGTDYSLTISNLEPEDIATYYCQQYSKLPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 180 - DNA, 1362 bp, synthetic construct
  source 1..1362: mol_type = other DNA; note = V9(VH-CL)-LC007(VL-CL)
gaggtgcagctggtggaatctggcggcggactggtgcagcctggcggatctctgagactgagctgtgccgccagcggctacagcttcaccggctacaccatgaactgggtgcgccaggcccctggcaagggactggaatgggtggccctgatcaacccctacaagggcgtgtccacctacaaccagaagttcaaggaccggttcaccatcagcgtggacaagagcaagaacaccgcctacctgcagatgaacagcctgcgggccgaggacaccgccgtgtactattgtgccagaagcggctactacggcgacagcgactggtacttcgacgtgtggggccagggcacactcgtgaccgtgtcaagcgctagcgtggccgctcccagcgtgttcatcttcccacctagcgacgagcagctgaagtccggcacagcctctgtcgtgtgcctgctgaacaacttctacccccgcgaggccaaggtgcagtggaaggtggacaatgccctgcagagcggcaacagccaggaaagcgtgaccgagcaggacagcaaggatagcacctacagcctgagcagcaccctgaccctgagcaaggccgactacgagaagcacaaggtgtacgcctgcgaagtgacccaccagggcctgtctagccccgtgaccaagagcttcaaccggggcgagtgtgatggcggaggcggatccgggggaggcggctctgatattgtgctgacccagagccccagcagcctgtctgcctctctgggcgacagagtgaccatcagctgtagcgcctctcagggcatccggaactacctgaactggtatcagcagcggcccgacggcaccgtgaagctgctgatctactacaccagctccctgcactccggcgtgcccagcagattttctggcagcggctccggcaccgactactccctgaccatctccaacctggaacccgaggatatcgccacctactactgccagcagtactccaagctgccctggacctttggaggcggcaccaagctggaaatcaagcgtacggtggctgccccctccgtgtttatctttcccccatccgatgaacagctgaaaagcggcaccgccagcgtcgtgtgtctgctgaacaatttttaccctagggaagctaaagtgcagtggaaagtggataacgcactgcagtccggcaactcccaggaatctgtgacagaacaggactctaaggacagcacatactccctgtcctccaccctgacactgtctaaggctgattatgagaaacacaaagtgtatgcttgtgaagtgacacatcagggactgagcagccctgtgacaaagtccttcaacagaggcgagtgc

SEQ ID NO: 181 - protein, 227 aa, synthetic construct
  source 1..227: note = Fc(knob) P329G LALA
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 182 - DNA, 681 bp, synthetic construct
  source 1..681: mol_type = other DNA; note = Fc(knob) P329G LALA
gacaaaactcacacatgcccaccgtgcccagcacctgaagctgcagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtacaccctgcccccatgccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 183 - protein, 212 aa, synthetic construct
  source 1..212: note = V9(VL-CH1)
DIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 184 - DNA, 636 bp, synthetic construct
  source 1..636: mol_type = other DNA; note = V9(VL-CH1)
gatattcagatgacccagagccccagctctctgagcgccagcgtgggcgacagagtgaccatcacctgtcgggccagccaggacatcagaaactacctgaactggtatcagcagaagcccggcaaggcccccaagctgctgatctactacaccagcagactggaaagcggcgtgccctccagattttccggcagcggctccggcaccgactacaccctgaccatcagcagcctgcagcccgaggatttcgccacatattactgccagcagggcaataccctgccctggaccttcggacagggcacaaaagtggaaatcaagagcagcgcttccaccaaaggcccttccgtgtttcctctggctcctagctccaagtccacctctggaggcaccgctgctctcggatgcctcgtgaaggattattttcctgagcctgtgacagtgtcctggaatagcggagcactgacctctggagtgcatactttccccgctgtgctgcagtcctctggactgtacagcctgagcagcgtggtgacagtgcccagcagcagcctgggcacccagacctacatctgcaacgtgaaccacaagcccagcaacaccaaggtggacaagaaggtggaacccaagtcttgt

SEQ ID NO: 185 - protein, 456 aa, synthetic construct
  source 1..456: note = V9(VH-CL)-Fc(knob) P329G LALA
EVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 186 - DNA, 1368 bp, synthetic construct
  source 1..1368: mol_type = other DNA; note = V9(VH-CL)-Fc(knob) P329G LALA
gaggtgcagctggtcgagagcggaggcggcctggtgcagcctggcggcagcctgagactgagctgcgccgccagcggctacagcttcaccggctacaccatgaactgggtccggcaggcacctggcaagggactggaatgggtggccctgatcaacccctacaagggcgtgagcacctacaaccagaagttcaaggaccggttcaccatcagcgtggacaagagcaagaacaccgcctatctgcagatgaacagcctgcgggccgaggacaccgccgtgtactactgcgccagaagcggctactacggcgacagcgactggtacttcgacgtgtggggccagggcaccctcgtgaccgtgtctagcgctagcgtggccgctccctccgtgtttatctttcccccatccgatgaacagctgaaaagcggcaccgcctccgtcgtgtgtctgctgaacaatttttaccctagggaagctaaagtgcagtggaaagtggataacgcactgcagtccggcaactcccaggaatctgtgacagaacaggactccaaggacagcacctactccctgtcctccaccctgacactgtctaaggctgattatgagaaacacaaagtctacgcctgcgaagtcacccatcagggcctgagctcgcccgtcacaaagagcttcaacaggggagagtgtgacaagacccacacctgtcccccttgtcctgcccctgaagctgctggcggcccttctgtgttcctgttccccccaaagcccaaggacaccctgatgatcagccggacccccgaagtgacctgcgtggtggtggatgtgtcccacgaggaccctgaagtgaagttcaattggtacgtggacggcgtggaagtgcacaacgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtacaccctgcccccatgccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 187 - protein, 682 aa, synthetic construct
  source 1..682: note = LC007(VH-CH1)-V9(VH-CL)-Fc(knob) P329G LALA
EVQLQESGPGLVKPSQSLSLTCSVTGYSITSGYYWNWIRQFPGNKLEWMGYITYDGSNNYNPSLKNRISITRDTSKNQFFLKLNSVTTEDTATYYCADFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 188 - DNA, 2046 bp, synthetic construct
  source 1..2046: mol_type = other DNA; note = LC007(VH-CH1)- V9(VH-CL)-Fc(knob) P329G LALA
gaggtgcagctgcaggaatctggccctggcctggtcaagccaagccagagtctgagcctgacctgcagcgtgaccggctacagcattaccagcggctactactggaactggattcggcagttccccggcaataagctggaatggatgggctacatcacctacgacggcagcaacaactacaaccccagcctgaagaaccggatcagcatcacccgggacaccagcaagaaccagttcttcctgaagctgaacagcgtgaccaccgaggacaccgccacatactattgcgccgacttcgactactggggccagggcaccaccctgaccgtgtccagcgccagcacaaagggccctagcgtgttccctctggcccccagcagcaagagcacaagcggcggaacagccgccctgggctgcctcgtgaaggactacttccccgagcccgtgacagtgtcttggaacagcggagccctgacaagcggcgtgcacaccttccctgccgtgctgcagagcagcggcctgtactccctgagcagcgtggtcaccgtgcctagcagcagcctgggcacccagacctacatctgcaacgtgaaccacaagcccagcaacaccaaagtggacaagaaggtggagcccaagagctgtgatggcggaggagggtccggaggcggaggatccgaagtgcagctggtggaatctggcggaggcctggtgcagcctggcggatctctgagactgagctgtgccgccagcggctacagcttcaccggctacaccatgaactgggtgcgccaggcccctggcaagggactggaatgggtggccctgatcaacccctacaagggcgtgtccacatacaaccagaagttcaaggaccggttcaccatcagcgtggacaagagcaagaacaccgcctacctgcagatgaacagcctgcgggccgaggacaccgccgtgtactattgtgccagaagcggctactacggcgacagcgactggtacttcgacgtgtggggccagggcacactcgtgaccgtgtcaagcgctagcgtggccgctccctccgtgtttatctttcccccatccgatgaacagctgaaaagcggcaccgcctccgtcgtgtgtctgctgaacaatttttaccctagggaagctaaagtgcagtggaaagtggataacgcactgcagtccggcaactcccaggaatctgtgacagaacaggactccaaggacagcacctactccctgtcctccaccctgacactgtctaaggctgattatgagaaacacaaagtctacgcctgcgaagtcacccatcagggcctgagctcgcccgtcacaaagagcttcaacaggggagagtgtgacaagacccacacctgtcccccttgtcctgcccctgaagctgctggcggcccttctgtgttcctgttccccccaaagcccaaggacaccctgatgatcagccggacccccgaagtgacctgcgtggtggtggatgtgtcccacgaggaccctgaagtgaagttcaattggtacgtggacggcgtggaagtgcacaacgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtacaccctgcccccatgccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 189 - protein, 214 aa, synthetic construct
  source 1..214: note = M4-3 ML2(VL-CL)
DIQMTQSPSSLSASVGDRVTITCRASQGIRNYLNWYQQKPGKAPKLLIYYTSSLHSGVPSRFSGSGSGTDFTLTISSLQPEDFATYYCQQYSKLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 190 - DNA, 642 bp, synthetic construct
  source 1..642: mol_type = other DNA; note = M4-3 ML2(VL-CL)
gacatccagatgacccagagccccagcagcctgagcgccagcgtgggcgacagagtgaccatcacctgccgggccagccagggcatccggaactacctgaactggtatcagcagaagcccggcaaggcccccaagctgctgatctactacaccagcagcctgcacagcggcgtgcctagccggtttagcggcagcggctccggcaccgacttcaccctgaccattagctccctgcagcccgaggacttcgccacctactactgccagcagtacagcaagctgccctggaccttcggccagggaacaaaggtggagatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 191 - protein, 664 aa, synthetic construct
  source 1..664: note = V9(VL-CH1)-M4-3 ML2(VH-CH1)-Fc(knob) P329G LALA
DIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSQVQLQESGPGLVKPSQTLSLTCTVSGGSITSGYYWNWIRQHPGKGLEWIGYITYDGSNNYNPSLKSRVTISRDTSKNQFSLKLSSVTAADTAVYYCADFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 192 - DNA, 1992 bp, synthetic construct
  source 1..1992: mol_type = other DNA; note = V9(VL-CH1)-M4-3 ML2(VH-CH1)-Fc(knob) P329G LALA (DNA)
gatatccagatgacccagagccccagctctctgagcgccagcgtgggcgacagagtgaccatcacctgtcgggccagccaggacatcagaaactacctgaactggtatcagcagaagcccggcaaggcccccaagctgctgatctactacaccagcagactggaaagcggcgtgccctccagattttccggcagcggctccggcaccgactacaccctgaccatcagcagcctgcagcccgaggatttcgccacatattactgccagcagggcaataccctgccctggaccttcggacagggcacaaaagtggaaatcaagagcagcgcttccaccaaaggcccttccgtgtttcctctggctcctagctccaagtccacctctggaggcaccgctgctctcggatgcctcgtgaaggattattttcctgagcctgtgacagtgtcctggaatagcggagcactgacctctggagtgcatactttccccgctgtgctgcagtcctctggactgtacagcctgagcagcgtggtgacagtgcccagcagcagcctgggcacccagacctacatctgcaacgtgaaccacaagcccagcaacaccaaggtggacaagaaggtggaacccaagtcttgtggcggaggcggatccggcggagggggatctcaggtgcagctgcaggaaagcggccctggcctggtcaagcccagccagaccctgagcctgacctgcaccgtgtccggcggcagcatcaccagcggctactactggaactggattcggcagcaccccggcaagggcctggaatggatcggctacatcacctacgacggcagcaacaactacaaccccagcctgaagtccagagtgaccatcagccgggacaccagcaagaaccagttcagcctgaagctgtccagcgtgacagccgccgacaccgccgtgtactactgcgccgacttcgactactggggccagggcaccctggtcaccgtgtccagcgctagcaccaagggccccagcgtgttccccctggcacccagcagcaagagcacatctggcggaacagccgctctgggctgtctggtgaaagactacttccccgagcccgtgaccgtgtcttggaactctggcgccctgaccagcggcgtgcacacctttccagccgtgctgcagagcagcggcctgtactccctgtcctccgtggtcaccgtgccctctagctccctgggaacacagacatatatctgtaatgtcaatcacaagccttccaacaccaaagtcgataagaaagtcgagcccaagagctgcgacaaaactcacacatgcccaccgtgcccagcacctgaagctgcagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtacaccctgcccccatgccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 193 - protein, 442 aa, synthetic construct
  source 1..442: note = M4-3 ML2(VH-CH1)-Fc(hole) P329G LALA
QVQLQESGPGLVKPSQTLSLTCTVSGGSITSGYYWNWIRQHPGKGLEWIGYITYDGSNNYNPSLKSRVTISRDTSKNQFSLKLSSVTAADTAVYYCADFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 194 - DNA, 1326 bp, synthetic construct
  source 1..1326: mol_type = other DNA; note = M4-3 ML2(VH-CH1)-Fc(hole) P329G LALA
caggtgcagctgcaggaaagcggccctggcctggtcaagcccagccagaccctgagcctgacctgcaccgtgtccggcggcagcatcaccagcggctactactggaactggatccggcagcaccccggcaagggcctggaatggatcggctacatcacctacgacggcagcaacaactacaaccccagcctgaagtccagagtgaccatcagccgggacaccagcaagaaccagttcagcctgaagctgtccagcgtgacagccgccgacaccgccgtgtactactgcgccgacttcgactactggggccagggcaccctggtcaccgtgtccagcgctagcaccaagggcccctccgtgttccccctggcccccagcagcaagagcaccagcggcggcacagccgctctgggctgcctggtcaaggactacttccccgagcccgtgaccgtgtcctggaacagcggagccctgacctccggcgtgcacaccttccccgccgtgctgcagagttctggcctgtatagcctgagcagcgtggtcaccgtgccttctagcagcctgggcacccagacctacatctgcaacgtgaaccacaagcccagcaacaccaaggtggacaagaaggtggagcccaagagctgcgacaaaactcacacatgcccaccgtgcccagcacctgaagctgcagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtgcaccctgcccccatcccgggatgagctgaccaagaaccaggtcagcctctcgtgcgcagtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 195 - protein, 681 aa, synthetic construct
  source 1..681: note = V9(VH-CL)-M4-3 ML2(VH-CH1)-Fc(knob) P329G LALA
EVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSQVQLQESGPGLVKPSQTLSLTCTVSGGSITSGYYWNWIRQHPGKGLEWIGYITYDGSNNYNPSLKSRVTISRDTSKNQFSLKLSSVTAADTAVYYCADFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 196 - DNA, 2043 bp, synthetic construct
  source 1..2043: mol_type = other DNA; note = V9(VH-CL)-M4-3 ML2(VH-CH1)-Fc(knob) P329G LALA (DNA)
gaggtgcagctggtcgagagcggaggcggcctggtgcagcctggcggcagcctgagactgagctgcgccgccagcggctacagcttcaccggctacaccatgaactgggtccggcaggcacctggcaagggactggaatgggtggccctgatcaacccctacaagggcgtgagcacctacaaccagaagttcaaggaccggttcaccatcagcgtggacaagagcaagaacaccgcctatctgcagatgaacagcctgcgggccgaggacaccgccgtgtactactgcgccagaagcggctactacggcgacagcgactggtacttcgacgtgtggggccagggcaccctcgtgaccgtgtctagcgctagcgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtggcggaggcggatccggcggagggggatctcaggtgcagctgcaggaaagcggccctggcctggtcaagcccagccagaccctgagcctgacctgcaccgtgtccggcggcagcatcaccagcggctactactggaactggattcggcagcaccccggcaagggcctggaatggatcggctacatcacctacgacggcagcaacaactacaaccccagcctgaagtccagagtgaccatcagccgggacaccagcaagaaccagttcagcctgaagctgtccagcgtgacagccgccgacaccgccgtgtactactgcgccgacttcgactactggggccagggcaccctggtcaccgtgtccagcgctagcaccaagggccccagcgtgttccccctggcacccagcagcaagagcacatctggcggaacagccgctctgggctgtctggtgaaagactacttccccgagcccgtgaccgtgtcttggaactctggcgccctgaccagcggcgtgcacacctttccagccgtgctgcagagcagcggcctgtactccctgtcctccgtggtcaccgtgccctctagctccctgggaacacagacatatatctgtaatgtcaatcacaagccttccaacaccaaagtcgataagaaagtcgagcccaagagctgcgacaaaactcacacatgcccaccgtgcccagcacctgaagctgcagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtacaccctgcccccatgccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 197 - protein, 690 aa, synthetic construct
  source 1..690: note = CH1A1A(VH-CH1)- V9(VH-CL)-Fc(knob) P329G LALA
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 198 - DNA, 2070 bp, synthetic construct
  source 1..2070: mol_type = other DNA; note = CH1A1A(VH-CH1)-V9(VH-CL)-Fc(knob) P329G LALA
caggtgcagctggtgcagtctggcgccgaagtgaagaaacctggcgccagcgtgaaggtgtcctgcaaggccagcggctacaccttcaccgagttcggcatgaactgggtccgacaggcccctggacagggcctggaatggatgggctggatcaacaccaagaccggcgaggccacctacgtggaagagttcaagggcagagtgaccttcaccaccgacaccagcaccagcaccgcctacatggaactgcggagcctgagaagcgacgacaccgccgtgtactactgcgccagatgggacttcgcctactatgtggaagccatggactactggggccagggcaccaccgtgaccgtgtctagtgctagcacaaagggccccagcgtgttccctctggcccctagcagcaagagcacatctggcggaacagccgccctgggctgcctggtcaaggactactttcccgagcccgtgacagtgtcctggaactctggcgccctgacaagcggcgtgcacacctttccagccgtgctgcagagcagcggcctgtactctctgagcagcgtggtcaccgtgcctagctctagcctgggcacccagacctacatctgcaacgtgaaccacaagcccagcaacaccaaggtggacaagaaggtggaacccaagagctgcggcggaggcggatccggaggcggaggatccgaagtgcagctggtggaatctggcggaggcctggtgcagcctggcggatctctgagactgagctgtgccgccagcggctacagcttcaccggctacaccatgaactgggtgcgccaggcccctggcaagggactggaatgggtggccctgatcaacccctacaagggcgtgtccacatacaaccagaagttcaaggaccggttcaccatcagcgtggacaagagcaagaacaccgcctacctgcagatgaacagcctgcgggccgaggacaccgccgtgtactattgtgccagaagcggctactacggcgacagcgactggtacttcgacgtgtggggccagggcacactcgtgaccgtgtcaagcgctagcgtggccgctccctccgtgtttatctttcccccatccgatgaacagctgaaaagcggcaccgcctccgtcgtgtgtctgctgaacaatttttaccctagggaagctaaagtgcagtggaaagtggataacgcactgcagtccggcaactcccaggaatctgtgacagaacaggactccaaggacagcacctactccctgtcctccaccctgacactgtctaaggctgattatgagaaacacaaagtctacgcctgcgaagtcacccatcagggcctgagctcgcccgtcacaaagagcttcaacaggggagagtgtgacaagacccacacctgtcccccttgtcctgcccctgaagctgctggcggcccttctgtgttcctgttccccccaaagcccaaggacaccctgatgatcagccggacccccgaagtgacctgcgtggtggtggatgtgtcccacgaggaccctgaagtgaagttcaattggtacgtggacggcgtggaagtgcacaacgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtacaccctgcccccatgccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 199 - protein, 214 aa, synthetic construct
  source 1..214: note = H2C(VL-CH1)
QTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 200 - DNA, 642 bp, synthetic construct
  source 1..642: mol_type = other DNA; note = H2C(VL-CH1)
cagaccgtggtgacacaggaacccagcctgaccgtctcccctggcggcaccgtgaccctgacctgtggaagcagcacaggcgccgtgaccagcggctactaccccaactgggtgcagcagaagcccggccaggcccctagaggactgatcggcggcaccaagtttctggcccctggcacccccgccagattctctggctctctgctgggcggcaaggccgccctgacactgtctggcgtgcagcctgaggacgaggccgagtactactgcgccctgtggtacagcaacagatgggtgttcggcggaggcaccaagctgaccgtgctgagcagcgcttccaccaaaggcccttccgtgtttcctctggctcctagctccaagtccacctctggaggcaccgctgctctcggatgcctcgtgaaggattattttcctgagcctgtgacagtgtcctggaatagcggagcactgacctctggagtgcatactttccccgctgtgctgcagtcctctggactgtacagcctgagcagcgtggtgacagtgcccagcagcagcctgggcacccagacctacatctgcaacgtgaaccacaagcccagcaacaccaaggtggacaagaaggtggaacccaagtcttgt

SEQ ID NO: 201 - protein, 684 aa, synthetic construct
  source 1..684: note = H2C(VH-CL)-M4-3 ML2(VH-CH1)-Fc(knob) P329G LALA
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSQVQLQESGPGLVKPSQTLSLTCTVSGGSITSGYYWNWIRQHPGKGLEWIGYITYDGSNNYNPSLKSRVTISRDTSKNQFSLKLSSVTAADTAVYYCADFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 202 - DNA, 2052 bp, synthetic construct
  source 1..2052: mol_type = other DNA; note = H2C(VH-CL)-M4-3 ML2(VH-CH1)-Fc(knob) P329G LALA
gaggtgcagctggtggaaagcggcggaggactggtgcagcctggcggaagcctgaagctgtcttgcgccgccagcggcttcaccttcaacaaatacgccatgaactgggtgcgccaggcccctggcaagggactggaatgggtggcccggatcagaagcaagtacaacaactacgccacctactacgccgacagcgtgaaggaccggttcaccatcagccgggacgacagcaagaacaccgcctacctgcagatgaacaacctgaaaaccgaggacaccgccgtgtactactgcgtgcggcacggcaacttcggcaacagctacatcagctactgggcctactggggacagggcaccctggtgacagtgtccagcgctagcgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtggcggaggcggatccggcggagggggatctcaggtgcagctgcaggaaagcggccctggcctggtcaagcccagccagaccctgagcctgacctgcaccgtgtccggcggcagcatcaccagcggctactactggaactggattcggcagcaccccggcaagggcctggaatggatcggctacatcacctacgacggcagcaacaactacaaccccagcctgaagtccagagtgaccatcagccgggacaccagcaagaaccagttcagcctgaagctgtccagcgtgacagccgccgacaccgccgtgtactactgcgccgacttcgactactggggccagggcaccctggtcaccgtgtccagcgctagcaccaagggccccagcgtgttccccctggcacccagcagcaagagcacatctggcggaacagccgctctgggctgtctggtgaaagactacttccccgagcccgtgaccgtgtcttggaactctggcgccctgaccagcggcgtgcacacctttccagccgtgctgcagagcagcggcctgtactccctgtcctccgtggtcaccgtgccctctagctccctgggaacacagacatatatctgtaatgtcaatcacaagccttccaacaccaaagtcgataagaaagtcgagcccaagagctgcgacaaaactcacacatgcccaccgtgcccagcacctgaagctgcagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtacaccctgcccccatgccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 203 - protein, 212 aa, synthetic construct
  source 1..212: note = 431/26(VL-CL)
DIQMTQSPSSLSASVGDRVTITCSTSSSVSYMHWYQQKPGKAPKLLIYSTSNLASGVPSRFSGSGSGTDFTFTISSLQPEDIATYYCHQWSSYPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 204 - DNA, 636 bp, synthetic construct
  source 1..636: mol_type = other DNA; note = 431/26(VL-CL)
gacatccagatgacccagagccccagcagcctgtctgccagcgtgggcgacagagtgaccatcacctgtagcaccagcagcagcgtgtcctacatgcactggtatcagcagaagcccggcaaggcccccaagctgctgatctacagcacctccaatctggccagcggcgtgcccagcagattttctggcagcggctccggcaccgacttcaccttcaccatcagctccctgcagcccgaggatatcgccacctactactgccaccagtggtccagctaccccacctttggccagggcaccaaggtggaaatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 205 - protein, 689 aa, synthetic construct
  source 1..689: note = 431/26(VH-CH1)-V9(VH-CL)-Fc(knob) P329G LALA
QVQLQESGPGLVRPSQTLSLTCTVSGFTISSGYSWHWVRQPPGRGLEWIGYIQYSGITNYNPSLKSRVTMLVDTSKNQFSLRLSSVTAADTAVYYCAREDYDYHWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 206 - DNA, 2067 bp, synthetic construct
  source 1..2067: mol_type = other DNA; note = 431/26(VH-CH1)-V9(VH-CL)-Fc(knob) P329G LALA
caggtgcagctgcaggaatctggccctggactcgtgcggcctagccagacactgagcctgacctgtaccgtgtccggcttcaccatcagcagcggctacagctggcattgggtgcgccagccacctggcagaggcctggaatggatcggctacatccagtacagcggcatcaccaactacaaccccagcctgaagtccagagtgaccatgctggtggacacctccaagaaccagttcagcctgcggctgagcagcgtgacagccgccgatacagccgtgtactactgcgccagagaggactacgactaccactggtacttcgacgtgtggggccagggctctctcgtgaccgtgtcaagcgctagcacaaagggccccagcgtgttccctctggcccctagcagcaagagcacatctggcggaacagccgccctgggctgcctggtcaaggactactttcccgagcccgtgacagtgtcctggaactctggcgccctgacaagcggcgtgcacacctttccagccgtgctgcagagcagcggcctgtactctctgagcagcgtggtcaccgtgcctagctctagcctgggcacccagacctacatctgcaacgtgaaccacaagcccagcaacaccaaggtggacaagaaggtggaacccaagagctgcggcggaggcggatccggaggcggaggatccgaagtgcagctggtggaatctggcggaggcctggtgcagcctggcggatctctgagactgagctgtgccgccagcggctacagcttcaccggctacaccatgaactgggtgcgccaggcccctggcaagggactggaatgggtggccctgatcaacccctacaagggcgtgtccacatacaaccagaagttcaaggaccggttcaccatcagcgtggacaagagcaagaacaccgcctacctgcagatgaacagcctgcgggccgaggacaccgccgtgtactattgtgccagaagcggctactacggcgacagcgactggtacttcgacgtgtggggccagggcacactcgtgaccgtgtcaagcgctagcgtggccgctccctccgtgtttatctttcccccatccgatgaacagctgaaaagcggcaccgcctccgtcgtgtgtctgctgaacaatttttaccctagggaagctaaagtgcagtggaaagtggataacgcactgcagtccggcaactcccaggaatctgtgacagaacaggactccaaggacagcacctactccctgtcctccaccctgacactgtctaaggctgattatgagaaacacaaagtctacgcctgcgaagtcacccatcagggcctgagctcgcccgtcacaaagagcttcaacaggggagagtgtgacaagacccacacctgtcccccttgtcctgcccctgaagctgctggcggcccttctgtgttcctgttccccccaaagcccaaggacaccctgatgatcagccggacccccgaagtgacctgcgtggtggtggatgtgtcccacgaggaccctgaagtgaagttcaattggtacgtggacggcgtggaagtgcacaacgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtacaccctgcccccatgccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 207 - protein, 450 aa, synthetic construct
  source 1..450: note = 431/26(VH-CH1)-Fc(hole) P329G LALA
QVQLQESGPGLVRPSQTLSLTCTVSGFTISSGYSWHWVRQPPGRGLEWIGYIQYSGITNYNPSLKSRVTMLVDTSKNQFSLRLSSVTAADTAVYYCAREDYDYHWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 208 - DNA, 1350 bp, synthetic construct
  source 1..1350: mol_type = other DNA; note = 431/26(VH-CH1)-Fc(hole) P329G LALA
caggtgcagctgcaggaatctggccctggactcgtgcggcctagccagacactgagcctgacctgtaccgtgtccggcttcaccatcagcagcggctacagctggcattgggtgcgccagccacctggcagaggcctggaatggatcggctacatccagtacagcggcatcaccaactacaaccccagcctgaagtccagagtgaccatgctggtggacacctccaagaaccagttcagcctgcggctgagcagcgtgacagccgccgatacagccgtgtactactgcgccagagaggactacgactaccactggtacttcgacgtgtggggccagggctctctcgtgaccgtgtcaagcgctagcaccaagggcccctccgtgttccccctggcccccagcagcaagagcaccagcggcggcacagccgctctgggctgcctggtcaaggactacttccccgagcccgtgaccgtgtcctggaacagcggagccctgacctccggcgtgcacaccttccccgccgtgctgcagagttctggcctgtatagcctgagcagcgtggtcaccgtgccttctagcagcctgggcacccagacctacatctgcaacgtgaaccacaagcccagcaacaccaaggtggacaagaaggtggagcccaagagctgcgacaaaactcacacatgcccaccgtgcccagcacctgaagctgcagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtgcaccctgcccccatcccgggatgagctgaccaagaaccaggtcagcctctcgtgcgcagtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 209 - protein, 464 aa, synthetic construct
  source 1..464: note = CH1A1A(VL-CL)-V9 (VH-CL)
DIQMTQSPSSLSASVGDRVTITCKASAAVGTYVAWYQQKPGKAPKLLIYSASYRKRGVPSRFSGSGSGTDFTLTISSLQPEDFATYYCHQYYTYPLFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 210 - DNA, 1392 bp, synthetic construct
  source 1..1392: mol_type = other DNA; note = CH1A1A(VL-CL)-V9 (VH-CL)
gatatccagatgacccagagccccagcagcctgtctgccagcgtgggcgacagagtgaccatcacatgcaaggcctctgccgccgtgggcacatacgtggcctggtatcagcagaagcccggcaaggcccccaagctgctgatctacagcgccagctaccggaagagaggcgtgcccagcagattttccggcagcggctctggcaccgacttcaccctgaccatcagctccctgcagcccgaggacttcgccacctactactgccaccagtactacacctaccccctgttcaccttcggccagggcaccaagctcgagatcaagcgtacggtggccgctcccagcgtgttcatcttcccacctagcgacgagcagctgaagtccggcacagcctctgtcgtgtgcctgctgaacaacttctacccccgcgaggccaaggtgcagtggaaggtggacaatgccctgcagagcggcaacagccaggaaagcgtgaccgagcaggacagcaaggactccacctacagcctgagcagcaccctgacactgagcaaggccgactacgagaagcacaaggtgtacgcctgcgaagtgacccaccagggcctgtctagccccgtgaccaagagcttcaaccggggcgaatgtggcggcggaggatccggcggaggcggctccggaggcggaggaagtggcggagggggatctgaagtgcagctggtggaatctggcggaggcctggtgcagcctggcggatctctgagactgagctgtgccgccagcggctacagcttcaccggctacaccatgaactgggtgcgccaggcccctggcaagggactggaatgggtggccctgatcaacccctacaagggcgtgtccacatacaaccagaagttcaaggaccggttcaccatcagcgtggacaagagcaagaacaccgcctacctgcagatgaacagcctgcgggccgaggacaccgccgtgtactactgtgccagaagcggctactacggcgacagcgactggtacttcgacgtgtggggccagggaaccctcgtgaccgtgtcaagcgctagcgtggccgcaccctctgtgtttatctttccaccctctgacgaacagctgaaaagcggcaccgccagcgtcgtgtgtctgctgaacaatttttaccctagggaagctaaagtgcagtggaaagtggataacgcactgcagtccggcaactcccaggaatctgtgacagaacaggactccaaggacagcacatactccctgtccagcacactgaccctgtctaaggccgattatgagaaacacaaagtgtatgcttgtgaagtgacacatcagggactgagcagccctgtgacaaagtccttcaacagaggcgagtgt

SEQ ID NO: 211 - protein, 451 aa, synthetic construct
  source 1..451: note = CH1A1A(VH-CH1)-Fc(knob) P329G LALA
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 212 - DNA, 1353 bp, synthetic construct
  source 1..1353: mol_type = other DNA; note = CH1A1A(VH-CH1)-Fc(knob) P329G LALA
caggtgcagctggtgcagtctggcgccgaagtgaagaaacctggagctagtgtgaaggtgtcctgcaaggccagcggctacaccttcaccgagttcggcatgaactgggtccgacaggctccaggccagggcctcgaatggatgggctggatcaacaccaagaccggcgaggccacctacgtggaagagttcaagggcagagtgaccttcaccacggacaccagcaccagcaccgcctacatggaactgcggagcctgagaagcgacgacaccgccgtgtactactgcgccagatgggacttcgcctattacgtggaagccatggactactggggccagggcaccaccgtgaccgtgtctagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagctgcagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtacaccctgcccccatgccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 213 - protein, 227 aa, synthetic construct
  source 1..227: note = Fc(hole) P329G LALA
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 214 - DNA, 681 bp, synthetic construct
  source 1..681: mol_type = other DNA; note = Fc(hole) P329G LALA
gacaaaactcacacatgcccaccgtgcccagcacctgaagctgcagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtgcaccctgcccccatcccgggatgagctgaccaagaaccaggtcagcctctcgtgcgcagtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 215 - protein, 214 aa, synthetic construct
  source 1..214: note = CH2527(VL-CH1)
QAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 216 - DNA, 642 bp, synthetic construct
  source 1..642: mol_type = other DNA; note = CH2527(VL-CH1)
caggccgtcgtgacccaggaaagcgccctgacaacaagccctggcgagacagtgaccctgacctgcagatctagcacaggcgccgtgaccaccagcaactacgccaactgggtgcaggaaaagcccgaccacctgttcaccggcctgatcggcggcaccaacaaaagggctccaggcgtgccagccagattcagcggcagcctgattggcgataaggccgccctgaccatcactggcgcccagacagaggacgaggccatctacttttgcgccctgtggtacagcaacctgtgggtgttcggcggaggcaccaagctgacagtgctgagcagcgcttccaccaaaggcccttccgtgtttcctctggctcctagctccaagtccacctctggaggcaccgctgctctcggatgcctcgtgaaggattattttcctgagcctgtgacagtgtcctggaatagcggagcactgacctctggagtgcatactttccccgctgtgctgcagtcctctggactgtacagcctgagcagcgtggtgacagtgcccagcagcagcctgggcacccagacctacatctgcaacgtgaaccacaagcccagcaacaccaaggtggacaagaaggtggaacccaagtcttgt

SEQ ID NO: 217 - protein, 684 aa, synthetic construct
  source 1..684: note = CH2527(VH-CL)-LC007(VH-CH1)-Fc(knob) P329G LALA
EVQLVESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSAASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSEVQLQESGPGLVKPSQSLSLTCSVTGYSITSGYYWNWIRQFPGNKLEWMGYITYDGSNNYNPSLKNRISITRDTSKNQFFLKLNSVTTEDTATYYCADFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 218 - DNA, 2052 bp, synthetic construct
  source 1..2052: mol_type = other DNA; note = CH2527(VH-CL)-LC007(VH-CH1)-Fc(knob) P329G LALA
gaagtgcagctggtggaaagcggcggaggcctggtgcagcctaagggctctctgaagctgagctgtgccgccagcggcttcaccttcaacacctacgccatgaactgggtgcgccaggcccctggcaaaggcctggaatgggtggcccggatcagaagcaagtacaacaattacgccacctactacgccgacagcgtgaaggaccggttcaccatcagccgggacgacagccagagcatcctgtacctgcagatgaacaacctgaaaaccgaggacaccgccatgtactactgcgtgcggcacggcaacttcggcaacagctatgtgtcttggtttgcctactggggccagggcaccctcgtgacagtgtctgctgctagcgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtggcggaggcggatccggcggagggggatctgaggtccagctgcaggagtcaggacctggcctcgtgaaaccttctcagtctctgtctctcacctgctctgtcactggctactccatcaccagtggttattactggaactggattcggcagtttccaggaaacaagctggaatggatgggctacataacctacgacggtagcaataactacaacccatctctcaaaaatcgaatctccattactcgtgacacatctaagaaccagtttttcctgaagttgaattctgtgactactgaggacacagctacatattactgtgcggactttgactactggggccaaggcaccactctcacagtctccagcgctagcaccaagggccccagcgtgttccccctggcacccagcagcaagagcacatctggcggaacagccgctctgggctgtctggtgaaagactacttccccgagcccgtgaccgtgtcttggaactctggcgccctgaccagcggcgtgcacacctttccagccgtgctgcagagcagcggcctgtactccctgtcctccgtggtcaccgtgccctctagctccctgggaacacagacatatatctgtaatgtcaatcacaagccttccaacaccaaagtcgataagaaagtcgagcccaagagctgcgacaaaactcacacatgcccaccgtgcccagcacctgaagctgcagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtacaccctgcccccatgccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 219 - protein, 685 aa, synthetic construct
  source 1..685: note = LC007(VH-CH1)-CH2527(VH-CL)-Fc(knob) P329G LALA
EVQLQESGPGLVKPSQSLSLTCSVTGYSITSGYYWNWIRQFPGNKLEWMGYITYDGSNNYNPSLKNRISITRDTSKNQFFLKLNSVTTEDTATYYCADFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLVESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSAASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 220 - DNA, 2055 bp, synthetic construct
  source 1..2055: mol_type = other DNA; note = LC007(VH-CH1)-CH2527(VH-CL)-Fc(knob) P329G LALA
gaggtgcagctgcaggaatctggccctggcctggtcaagccaagccagagtctgagcctgacctgcagcgtgaccggctacagcattaccagcggctactactggaactggattcggcagttccccggcaataagctggaatggatgggctacatcacctacgacggcagcaacaactacaaccccagcctgaagaaccggatcagcatcacccgggacaccagcaagaaccagttcttcctgaagctgaacagcgtgaccaccgaggacaccgccacatactattgcgccgacttcgactactggggccagggcaccaccctgaccgtgtccagcgccagcacaaagggccctagcgtgttccctctggcccccagcagcaagagcacaagcggcggaacagccgccctgggctgcctcgtgaaggactacttccccgagcccgtgacagtgtcttggaacagcggagccctgacaagcggcgtgcacaccttccctgccgtgctgcagagcagcggcctgtactccctgagcagcgtggtcaccgtgcctagcagcagcctgggcacccagacctacatctgcaacgtgaaccacaagcccagcaacaccaaagtggacaagaaggtggagcccaagagctgtgatggcggaggagggtccggaggcggaggatccgaagtgcagctggtggaaagcggcggaggcctggtgcagcctaagggctctctgaagctgagctgtgccgccagcggcttcaccttcaacacctacgccatgaactgggtgcgccaggcccctggcaaaggcctggaatgggtggcccggatcagaagcaagtacaacaattacgccacctactacgccgacagcgtgaaggaccggttcaccatcagccgggacgacagccagagcatcctgtacctgcagatgaacaacctgaaaaccgaggacaccgccatgtactactgcgtgcggcacggcaacttcggcaacagctatgtgtcttggtttgcctactggggccagggcaccctcgtgacagtgtctgctgctagcgtggccgctccctccgtgtttatctttcccccatccgatgaacagctgaaaagcggcaccgcctccgtcgtgtgtctgctgaacaatttttaccctagggaagctaaagtgcagtggaaagtggataacgcactgcagtccggcaactcccaggaatctgtgacagaacaggactccaaggacagcacctactccctgtcctccaccctgacactgtctaaggctgattatgagaaacacaaagtctacgcctgcgaagtcacccatcagggcctgagctcgcccgtcacaaagagcttcaacaggggagagtgtgacaagacccacacctgtcccccttgtcctgcccctgaagctgctggcggcccttctgtgttcctgttccccccaaagcccaaggacaccctgatgatcagccggacccccgaagtgacctgcgtggtggtggatgtgtcccacgaggaccctgaagtgaagttcaattggtacgtggacggcgtggaagtgcacaacgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtacaccctgcccccatgccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 221 - protein, 218 aa, synthetic construct
  source 1..218: note = anti-CD33(VL-CL)
DIQMTQSPSSLSASVGDRVTITCRASESVDNYGISFMNWFQQKPGKAPKLLIYAASNQGSGVPSRFSGSGSGTDFTLTISSLQPDDFATYYCQQSKEVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 222 - DNA, 654 bp, synthetic construct
  source 1..654: mol_type = other DNA; note = anti-CD33(VL-CL)
gacatccagatgacccagagccccagcagcctgagcgccagcgtgggcgacagagtgaccatcacctgtcgggccagcgagagcgtggacaactacggcatcagcttcatgaactggttccagcagaagcccggcaaggcccccaagctgctgatctacgccgccagcaatcagggcagcggcgtgcccagcagattcagcggctctggcagcggcaccgacttcaccctgaccatcagcagcctgcagcccgacgacttcgccacctactactgccagcagagcaaagaggtgccctggaccttcggccagggcaccaaggtggaaatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 223 - protein, 668 aa, synthetic construct
  source 1..668: note = V9(VL-CH1)-anti-CD33(VH-CH1)-Fc(knob) P329G LALA
DIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSQVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 224 - DNA, 2004 bp, synthetic construct
  source 1..2004: mol_type = other DNA; note = V9(VL-CH1)-anti-CD33(VH-CH1)-Fc(knob) P329G LALA
gatatccagatgacccagagccccagctctctgagcgccagcgtgggcgacagagtgaccatcacctgtcgggccagccaggacatcagaaactacctgaactggtatcagcagaagcccggcaaggcccccaagctgctgatctactacaccagcagactggaaagcggcgtgccctccagattttccggcagcggctccggcaccgactacaccctgaccatcagcagcctgcagcccgaggatttcgccacatattactgccagcagggcaataccctgccctggaccttcggacagggcacaaaagtggaaatcaagagcagcgcttccaccaaaggcccttccgtgtttcctctggctcctagctccaagtccacctctggaggcaccgctgctctcggatgcctcgtgaaggattattttcctgagcctgtgacagtgtcctggaatagcggagcactgacctctggagtgcatactttccccgctgtgctgcagtcctctggactgtacagcctgagcagcgtggtgacagtgcccagcagcagcctgggcacccagacctacatctgcaacgtgaaccacaagcccagcaacaccaaggtggacaagaaggtggaacccaagtcttgtggcggaggcggatccggcggaggcggatctcaggtgcagctggtgcagtctggcgccgaagtgaagaaacccggcagcagcgtgaaggtgtcctgcaaggccagcggctacaccttcaccgactacaacatgcactgggtccgccaggccccaggccagggactggaatggatcggctacatctacccctacaacggcggcaccggctacaaccagaagttcaagagcaaggccaccatcaccgccgacgagagcaccaacaccgcctacatggaactgagcagcctgcggagcgaggacaccgccgtgtactactgcgccagaggcagacccgccatggactactggggccagggcaccctggtgacagtgtccagcgccagcacaaagggccccagcgtgttccccctggcacccagcagcaagagcacatctggcggaacagccgctctgggctgtctggtgaaagactacttccccgagcccgtgaccgtgtcttggaactctggcgccctgaccagcggcgtgcacacctttccagccgtgctgcagagcagcggcctgtactccctgtcctccgtggtcaccgtgccctctagctccctgggaacacagacatatatctgtaatgtcaatcacaagccttccaacaccaaagtcgataagaaagtcgagcccaagagctgcgacaaaactcacacatgcccaccgtgcccagcacctgaagctgcagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtacaccctgcccccatgccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 225 - protein, 446 aa, synthetic construct
  source 1..446: note = anti-CD33(VH-CH1)-Fc(hole) P329G LALA
QVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 226 - DNA, 1338 bp, synthetic construct
  source 1..1338: mol_type = other DNA; note = anti-CD33(VH-CH1)-Fc(hole) P329G LALA
caggtgcagctggtgcagtctggcgccgaagtgaagaaacccggcagcagcgtgaaggtgtcctgcaaggccagcggctacaccttcaccgactacaacatgcactgggtccgccaggccccaggccagggactggaatggatcggctacatctacccctacaacggcggcaccggctacaaccagaagttcaagagcaaggccaccatcaccgccgacgagagcaccaacaccgcctacatggaactgagcagcctgcggagcgaggacaccgccgtgtactactgcgccagaggcagacccgccatggactactggggccagggcaccctggtgacagtgtccagcgctagcaccaagggcccctccgtgttccccctggcccccagcagcaagagcaccagcggcggcacagccgctctgggctgcctggtcaaggactacttccccgagcccgtgaccgtgtcctggaacagcggagccctgacctccggcgtgcacaccttccccgccgtgctgcagagttctggcctgtatagcctgagcagcgtggtcaccgtgccttctagcagcctgggcacccagacctacatctgcaacgtgaaccacaagcccagcaacaccaaggtggacaagaaggtggagcccaagagctgcgacaaaactcacacatgcccaccgtgcccagcacctgaagctgcagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtgcaccctgcccccatcccgggatgagctgaccaagaaccaggtcagcctctcgtgcgcagtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 227 - protein, 219 aa, synthetic construct
  source 1..219: note = anti-CD20(VL-CL)
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 228 - DNA, 657 bp, synthetic construct
  source 1..657: mol_type = other DNA; note = anti-CD20(VL-CL)
gatatcgtgatgacccagactccactctccctgcccgtcacccctggagagcccgccagcattagctgcaggtctagcaagagcctcttgcacagcaatggcatcacttatttgtattggtacctgcaaaagccagggcagtctccacagctcctgatttatcaaatgtccaaccttgtctctggcgtccctgaccggttctccggatccgggtcaggcactgatttcacactgaaaatcagcagggtggaggctgaggatgttggagtttattactgcgctcagaatctagaacttccttacaccttcggcggagggaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 229 - protein, 671 aa, synthetic construct
  source 1..671: note = V9(VL-CH1)-anti-CD20(VH-CH1)-Fc(knob) P329G LALA
DIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSQVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 230 - DNA, 2013 bp, synthetic construct
  source 1..2013: mol_type = other DNA; note = V9(VL-CH1)-anti-CD20(VH-CH1)-Fc(knob) P329G LALA
gatatccagatgacccagagccccagctctctgagcgccagcgtgggcgacagagtgaccatcacctgtcgggccagccaggacatcagaaactacctgaactggtatcagcagaagcccggcaaggcccccaagctgctgatctactacaccagcagactggaaagcggcgtgccctccagattttccggcagcggctccggcaccgactacaccctgaccatcagcagcctgcagcccgaggatttcgccacatattactgccagcagggcaataccctgccctggaccttcggacagggcacaaaagtggaaatcaagagcagcgcttccaccaaaggcccttccgtgtttcctctggctcctagctccaagtccacctctggaggcaccgctgctctcggatgcctcgtgaaggattattttcctgagcctgtgacagtgtcctggaatagcggagcactgacctctggagtgcatactttccccgctgtgctgcagtcctctggactgtacagcctgagcagcgtggtgacagtgcccagcagcagcctgggcacccagacctacatctgcaacgtgaaccacaagcccagcaacaccaaggtggacaagaaggtggaacccaagtcttgtggcggaggcggatccggcggagggggatctcaggtgcaattggtgcagtctggcgctgaagttaagaagcctgggagttcagtgaaggtctcctgcaaggcttccggatacgccttcagctattcttggatcaattgggtgcggcaggcgcctggacaagggctcgagtggatgggacggatctttcccggcgatggggatactgactacaatgggaaattcaagggcagagtcacaattaccgccgacaaatccactagcacagcctatatggagctgagcagcctgagatctgaggacacggccgtgtattactgtgcaagaaatgtctttgatggttactggcttgtttactggggccagggaaccctggtcaccgtctcctcagctagcaccaagggccccagcgtgttccccctggcacccagcagcaagagcacatctggcggaacagccgctctgggctgtctggtgaaagactacttccccgagcccgtgaccgtgtcttggaactctggcgccctgaccagcggcgtgcacacctttccagccgtgctgcagagcagcggcctgtactccctgtcctccgtggtcaccgtgccctctagctccctgggaacacagacatatatctgtaatgtcaatcacaagccttccaacaccaaagtcgataagaaagtcgagcccaagagctgcgacaaaactcacacatgcccaccgtgcccagcacctgaagctgcagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtacaccctgcccccatgccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 231 - protein, 449 aa, synthetic construct
  source 1..449: note = anti-CD20(VH-CH1)-Fc(hole) P329G LALA
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 232 - DNA, 1347 bp, synthetic construct
  source 1..1347: mol_type = other DNA; note = anti-CD20(VH-CH1)-Fc(hole) P329G LALA
caggtgcaattggtgcagtctggcgctgaagttaagaagcctgggagttcagtgaaggtctcctgcaaggcttccggatacgccttcagctattcttggatcaattgggtgcggcaggcgcctggacaagggctcgagtggatgggacggatctttcccggcgatggggatactgactacaatgggaaattcaagggcagagtcacaattaccgccgacaaatccactagcacagcctatatggagctgagcagcctgagatctgaggacacggccgtgtattactgtgcaagaaatgtctttgatggttactggcttgtttactggggccagggaaccctggtcaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagctgcagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtgcaccctgcccccatcccgggatgagctgaccaagaaccaggtcagcctctcgtgcgcagtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 233 - protein, 11 aa, synthetic construct
  source 1..11: note = M4-3 ML2 HCDR1
GGSITSGYYWN

SEQ ID NO: 234 - DNA, 33 bp, synthetic construct
  source 1..33: mol_type = other DNA; note = M4-3 ML2 HCDR1
ggcggcagcatcaccagcggctactactggaac

SEQ ID NO: 235 - protein, 16 aa, synthetic construct
  source 1..16: note = M4-3 ML2 HCDR2
YITYDGSNNYNPSLKS

SEQ ID NO: 236 - DNA, 48 bp, synthetic construct
  source 1..48: mol_type = other DNA; note = M4-3 ML2 HCDR2
tacatcacctacgacggcagcaacaactacaaccccagcctgaagtcc

SEQ ID NO: 237-238 - skipped (2 entries)

SEQ ID NO: 239 - protein, 112 aa, synthetic construct
  source 1..112: note = M4-3 ML2 VH
QVQLQESGPGLVKPSQTLSLTCTVSGGSITSGYYWNWIRQHPGKGLEWIGYITYDGSNNYNPSLKSRVTISRDTSKNQFSLKLSSVTAADTAVYYCADFDYWGQGTLVTVSS

SEQ ID NO: 240 - DNA, 336 bp, synthetic construct
  source 1..336: mol_type = other DNA; note = M4-3 ML2 VH
caggtgcagctgcaggaaagcggccctggcctggtcaagcccagccagaccctgagcctgacctgcaccgtgtccggcggcagcatcaccagcggctactactggaactggatccggcagcaccccggcaagggcctggaatggatcggctacatcacctacgacggcagcaacaactacaaccccagcctgaagtccagagtgaccatcagccgggacaccagcaagaaccagttcagcctgaagctgtccagcgtgacagccgccgacaccgccgtgtactactgcgccgacttcgactactggggccagggcaccctggtcaccgtgtccagc

SEQ ID NO: 241 - protein, 11 aa, synthetic construct
  source 1..11: note = M4-3 ML2 LCDR1
RASQGIRNYLN

SEQ ID NO: 242 - DNA, 33 bp, synthetic construct
  source 1..33: mol_type = other DNA; note = M4-3 ML2 LCDR1
cgggccagccagggcatccggaactacctgaac

SEQ ID NO: 243 - protein, 7 aa, synthetic construct
  source 1..7: note = M4-3 ML2 LCDR2
YTSSLHS

SEQ ID NO: 244 - DNA, 21 bp, synthetic construct
  source 1..21: mol_type = other DNA; note = M4-3 ML2 LCDR2
tacaccagcagcctgcacagc

SEQ ID NO: 245 - protein, 9 aa, synthetic construct
  source 1..9: note = M4-3 ML2 LCDR3
QQYSKLPWT

SEQ ID NO: 246 - DNA, 27 bp, synthetic construct
  source 1..27: mol_type = other DNA; note = M4-3 ML2 LCDR3
cagcagtacagcaagctgccctggacc

SEQ ID NO: 247 - protein, 107 aa, synthetic construct
  source 1..107: note = M4-3 ML2 VL
DIQMTQSPSSLSASVGDRVTITCRASQGIRNYLNWYQQKPGKAPKLLIYYTSSLHSGVPSRFSGSGSGTDFTLTISSLQPEDFATYYCQQYSKLPWTFGQGTKVEIK

SEQ ID NO: 248 - DNA, 321 bp, synthetic construct
  source 1..321: mol_type = other DNA; note = M4-3 ML2 VL
gacatccagatgacccagagccccagcagcctgagcgccagcgtgggcgacagagtgaccatcacctgccgggccagccagggcatccggaactacctgaactggtatcagcagaagcccggcaaggcccccaagctgctgatctactacaccagcagcctgcacagcggcgtgcctagccggtttagcggcagcggctccggcaccgacttcaccctgaccattagctccctgcagcccgaggacttcgccacctactactgccagcagtacagcaagctgccctggaccttcggccagggaacaaaggtggagatcaag

SEQ ID NO: 249 - protein, 5 aa, synthetic construct
  source 1..5: note = anti-CD3 HCDR1
TYAMN

SEQ ID NO: 250 - DNA, 15 bp, synthetic construct
  source 1..15: mol_type = other DNA; note = anti-CD3 HCDR1
acctacgccatgaac

SEQ ID NO: 251 - protein, 19 aa, synthetic construct
  source 1..19: note = anti-CD3 HCDR2
RIRSKYNNYATYYADSVKD

SEQ ID NO: 252 - DNA, 57 bp, synthetic construct
  source 1..57: mol_type = other DNA; note = anti-CD3 HCDR2
cggatcagaagcaagtacaacaattacgccacctactacgccgacagcgtgaaggac

SEQ ID NO: 253 - protein, 14 aa, synthetic construct
  source 1..14: note = anti-CD3 HCDR3
HGNFGNSYVSWFAY

SEQ ID NO: 254 - DNA, 42 bp, synthetic construct
  source 1..42: mol_type = other DNA; note = anti-CD3 HCDR3
cacggcaacttcggcaacagctatgtgtcttggtttgcctac

SEQ ID NO: 255 - protein, 125 aa, synthetic construct
  source 1..125: note = anti-CD3 VH
EVKLLESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSA

SEQ ID NO: 256 - DNA, 375 bp, synthetic construct
  source 1..375: mol_type = other DNA; note = anti-CD3 VH
gaagtgcagctggtggaaagcggcggaggcctggtgcagcctaagggctctctgaagctgagctgtgccgccagcggcttcaccttcaacacctacgccatgaactgggtgcgccaggcccctggcaaaggcctggaatgggtggcccggatcagaagcaagtacaacaattacgccacctactacgccgacagcgtgaaggaccggttcaccatcagccgggacgacagccagagcatcctgtacctgcagatgaacaacctgaaaaccgaggacaccgccatgtactactgcgtgcggcacggcaacttcggcaacagctatgtgtcttggtttgcctactggggccagggcaccctcgtgacagtgtctgct

SEQ ID NO: 257 - protein, 14 aa, synthetic construct
  source 1..14: note = anti-CD3 LCDR1
RSSTGAVTTSNYAN

SEQ ID NO: 258 - DNA, 42 bp, synthetic construct
  source 1..42: mol_type = other DNA; note = anti-CD3 LCDR1
agatctagcacaggcgccgtgaccaccagcaactacgccaac

SEQ ID NO: 259 - protein, 7 aa, synthetic construct
  source 1..7: note = anti-CD3 LCDR2
GTNKRAP

SEQ ID NO: 260 - DNA, 21 bp, synthetic construct
  source 1..21: mol_type = other DNA; note = anti-CD3 LCDR2
ggcaccaacaaaagggctcca

SEQ ID NO: 261 - protein, 9 aa, synthetic construct
  source 1..9: note = anti-CD3 LCDR3
ALWYSNLWV

SEQ ID NO: 262 - DNA, 27 bp, synthetic construct
  source 1..27: mol_type = other DNA; note = anti-CD3 LCDR3
gccctgtggtacagcaacctgtgggtg

SEQ ID NO: 263 - protein, 109 aa, synthetic construct
  source 1..109: note = anti-CD3 VL
QAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVL

SEQ ID NO: 264 - DNA, 327 bp, synthetic construct
  source 1..327: mol_type = other DNA; note = anti-CD3 VL
caggccgtcgtgacccaggaaagcgccctgacaacaagccctggcgagacagtgaccctgacctgcagatctagcacaggcgccgtgaccaccagcaactacgccaactgggtgcaggaaaagcccgaccacctgttcaccggcctgatcggcggcaccaacaaaagggctccaggcgtgccagccagattcagcggcagcctgattggcgataaggccgccctgaccatcactggcgcccagacagaggacgaggccatctacttttgcgccctgtggtacagcaacctgtgggtgttcggcggaggcaccaagctgacagtgctg

SEQ ID NO: 265 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 266 - protein, 198 aa, Macaca fascicularis
MQSGTRWRVLGLCLLSIGVWGQDGNEEMGSITQTPYQVSISGTTVILTCSQHLGSEAQWQHNGKNKEDSGDRLFLPEFSEMEQSGYYVCYPRGSNPEDASHHLYLKARVCENCMEMDVMAVATIVIVDICITLGLLLLVYYWSKNRKAKAKPVTRGAGAGGRQRGQNKERPPPVPNPDYEPIRKGQQDLYSGLNQRRI

(19) United States
(12) Patent Application Publication
(10) Pub. No.: US 2023/0340160 A1
(43) Pub. Date: Oct. 26, 2023

(54) BISPECIFIC T CELL ACTIVATING ANTIGEN BINDING MOLECULES

(71) Applicant: Roche Glycart AG, Schlieren (CH)

(72) Inventors: Oliver AST, Bassersdorf (CH); Peter BRUENKER, Hittnau (CH); Tanja FAUTI, Zuerich (CH); Anne FREIMOSER-GRUNDSCHOBER, Zuerich (CH); Christiane NEUMANN, Wallisellen (CH); Christian KLEIN, Bonstetten (CH); Ekkehard MOESSNER, Kreuzlingen (CH); Pablo UMANA, Wollerau (CH)

(21) Appl. No.: 18/301,789

(22) Filed: Apr. 17, 2023

Related U.S. Application Data
(63) Continuation of application No. 17/872,538, filed on Jul. 25, 2022, now abandoned.
Application No. 17/872,538 is a continuation of application No. 15/918,034, filed on Mar. 12, 2018, now abandoned.
Application No. 15/918,034 is a continuation of application No. 13/590,886, filed on Aug. 21, 2012, now abandoned.

(30) Foreign Application Priority Data
Aug. 23, 2011 (EP) 11178370.0
May 16, 2012 (EP) 12168192.8

(51) Int. Cl. C07K 16/30; C07K 16/28; C07K 16/46; C07K 16/40

(52) U.S. Cl. CPC C07K 16/468; C07K 16/2809; C07K 16/2863; C07K 16/3007; C07K 16/3053; C07K 16/40; C07K 2317/31; C07K 2317/52; C07K 2317/55; C07K 2317/626; C07K 2317/64; C07K 2317/66; C07K 2317/73; C07K 2317/92; C07K 2317/94; C07K 2319/00

(57) ABSTRACT

The present invention generally relates to novel bispecific antigen binding molecules for T cell activation and re-direction to specific target cells. In addition, the present invention relates to polynucleotides encoding such bispecific antigen binding molecules, and vectors and host cells comprising such polynucleotides. The invention further relates to methods for producing the bispecific antigen binding molecules of the invention, and to methods of using these bispecific antigen binding molecules in the treatment of disease.

### CROSS-REFERENCE TO RELATED APPLICATIONS

This application is a continuation of U.S. Pat. Application No. 17/872,538, filed on Jul. 25, 2022, which is a continuation of U.S. Pat. Application No. 15/918,034, filed on Mar. 12, 2018, which is a continuation of U.S. Pat. Application No. 13/590,886, filed Aug. 21, 2012, which claims priority to European Patent Application No. EP 11178370.0, filed Aug. 23, 2011, and to European Patent Application No. EP 12168192.8, filed May 16, 2012, the disclosures of which are incorporated herein by reference in their entirety.

### SEQUENCE LISTING

The instant application contains a Sequence Listing which has been submitted electronically in XML file format and is hereby incorporated by reference in its entirety. Said XML copy, created on Mar. 29, 2023, is named 51177-003004_Sequence_Listing_3_29_23 and is 407,220 bytes in size.

### FIELD OF THE INVENTION

The present invention generally relates to bispecific antigen binding molecules for activating T cells. In addition, the present invention relates to polynucleotides encoding such bispecific antigen binding molecules, and vectors and host cells comprising such polynucleotides. The invention further relates to methods for producing the bispecific antigen binding molecules of the invention, and to methods of using these bispecific antigen binding molecules in the treatment of disease.

### BACKGROUND

The selective destruction of an individual cell or a specific cell type is often desirable in a variety of clinical settings. For example, it is a primary goal of cancer therapy to specifically destroy tumor cells, while leaving healthy cells and tissues intact and undamaged.

An attractive way of achieving this is by inducing an immune response against the tumor, to make immune effector cells such as natural killer (NK) cells or cytotoxic T lymphocytes (CTLs) attack and destroy tumor cells. CTLs constitute the most potent effector cells of the immune system, however they cannot be activated by the effector mechanism mediated by the Fc domain of conventional therapeutic antibodies.

In this regard, bispecific antibodies designed to bind with one “arm” to a surface antigen on target cells, and with the second “arm” to an activating, invariant component of the T cell receptor (TCR) complex, have become of interest in recent years. The simultaneous binding of such an antibody to both of its targets will force a temporary interaction between target cell and T cell, causing activation of any cytotoxic T cell and subsequent lysis of the target cell. Hence, the immune response is re-directed to the target cells and is independent of peptide antigen presentation by the target cell or the specificity of the T cell as would be relevant for normal MHC-restricted activation of CTLs. In this context it is crucial that CTLs are only activated when a target cell is presenting the bispecific antibody to them, i.e. the immunological synapse is mimicked. Particularly desirable are bispecific antibodies that do not require lymphocyte preconditioning or co-stimulation in order to elicit efficient lysis of target cells.

Several bispecific antibody formats have been developed and their suitability for T cell mediated immunotherapy investigated. Out of these, the so-called BiTE (bispecific T cell engager) molecules have been very well characterized and already shown some promise in the clinic (reviewed in Nagorsen and Bäuerle, Exp Cell Res 317, 1255-1260 (2011)). BiTEs are tandem scFv molecules wherein two scFv molecules are fused by a flexible linker. Further bispecific formats being evaluated for T cell engagement include diabodies (Holliger et al., Prot Eng 9, 299-305 (1996)) and derivatives thereof, such as tandem diabodies (Kipriyanov et al., J Mol Biol 293, 41-66 (1999)). A more recent development are the so-called DART (dual affinity retargeting) molecules, which are based on the diabody format but feature a C-terminal disulfide bridge for additional stabilization (Moore et al., Blood 117, 4542-51 (2011)). The so-called triomabs, which are whole hybrid mouse/rat IgG molecules and also currently being evaluated in clinical trials, represent a larger sized format (reviewed in Seimetz et al., Cancer Treat Rev 36, 458-467 (2010)).

The variety of formats that are being developed shows the great potential attributed to T cell re-direction and activation in immunotherapy. The task of generating bispecific antibodies suitable therefor is, however, by no means trivial, but involves a number of challenges that have to be met related to efficacy, toxicity, applicability and produceability of the antibodies.

Small constructs such as, for example, BiTE molecules – while being able to efficiently crosslink effector and target cells – have a very short serum half life requiring them to be administered to patients by continuous infusion. IgG-like formats on the other hand – while having the great benefit of a long half life – suffer from toxicity associated with the native effector functions inherent to IgG molecules. Their immunogenic potential constitutes another unfavorable feature of IgG-like bispecific antibodies, especially non-human formats, for successful therapeutic development. Finally, a major challenge in the general development of bispecific antibodies has been the production of bispecific antibody constructs at a clinically sufficient quantity and purity, due to the mispairing of antibody heavy and light chains of different specificities upon co-expression, which decreases the yield of the correctly assembled construct and results in a number of non-functional side products from which the desired bispecific antibody may be difficult to separate.

Given the difficulties and disadvantages associated with currently available bispecific antibodies for T cell mediated immunotherapy, there remains a need for novel, improved formats of such molecules. The present invention provides bispecific antigen binding molecules designed for T cell activation and re-direction that combine good efficacy and produceability with low toxicity and favorable pharmacokinetic properties.

### SUMMARY OF THE INVENTION

In a first aspect the present invention provides a T cell activating bispecific antigen binding molecule comprising a first and a second antigen binding moiety, one of which is a Fab molecule capable of specific binding to an activating T cell antigen and the other one of which is a Fab molecule capable of specific binding to a target cell antigen, and an Fc domain composed of a first and a second subunit capable of stable association; wherein the first antigen binding moiety is (a) a single chain Fab molecule wherein the Fab light chain and the Fab heavy chain are connected by a peptide linker, or (b) a crossover Fab molecule wherein either the variable or the constant regions of the Fab light chain and the Fab heavy chain are exchanged.

In a particular embodiment, not more than one antigen binding moiety capable of specific binding to an activating T cell antigen is present in the T cell activating bispecific antigen binding molecule (i.e. the T cell activating bispecific antigen binding molecule provides monovalent binding to the activating T cell antigen). In particular embodiments, the first antigen binding moiety is a crossover Fab molecule. In even more particular embodiments, the first antigen binding moiety is a crossover Fab molecule wherein the constant regions of the Fab light chain and the Fab heavy chain are exchanged.

In some embodiments, the first and the second antigen binding moiety of the T cell activating bispecific antigen binding molecule are fused to each other, optionally via a peptide linker. In one such embodiment, the second antigen binding moiety is fused at the C-terminus of the Fab heavy chain to the N-terminus of the Fab heavy chain of the first antigen binding moiety. In another such embodiment, the first antigen binding moiety is fused at the C-terminus of the Fab heavy chain to the N-terminus of the Fab heavy chain of the second antigen binding moiety. In yet another such embodiment, the second antigen binding moiety is fused at the C-terminus of the Fab light chain to the N-terminus of the Fab light chain of the first antigen binding moiety. In embodiments wherein the first antigen binding moiety is a crossover Fab molecule and wherein either (i) the second antigen binding moiety is fused at the C-terminus of the Fab heavy chain to the N-terminus of the Fab heavy chain of the first antigen binding moiety or (ii) the first antigen binding moiety is fused at the C-terminus of the Fab heavy chain to the N-terminus of the Fab heavy chain of the second antigen binding moiety, additionally the Fab light chain of the first antigen binding moiety and the Fab light chain of the second antigen binding moiety may be fused to each other, optionally via a peptide linker.

In one embodiment, the second antigen binding moiety of the T cell activating bispecific antigen binding molecule is fused at the C-terminus of the Fab heavy chain to the N-terminus of the first or the second subunit of the Fc domain. In another embodiment, the first antigen binding moiety is fused at the C-terminus of the Fab heavy chain to the N-terminus of the first or second subunit of the Fc domain.

In one embodiment, the first and the second antigen binding moiety of the T cell activating bispecific antigen binding molecule are each fused at the C-terminus of the Fab heavy chain to the N-terminus of one of the subunits of the Fc domain.

In certain embodiments, the T cell activating bispecific antigen binding molecule comprises a third antigen binding moiety which is a Fab molecule capable of specific binding to a target cell antigen. In one such embodiment, the third antigen binding moiety is fused at the C-terminus of the Fab heavy chain to the N-terminus of the first or second subunit of the Fc domain. In a particular embodiment, the second and the third antigen binding moiety of the T cell activating antigen binding molecule are each fused at the C-terminus of the Fab heavy chain to the N-terminus of one of the subunits of the Fc domain, and the first antigen binding moiety is fused at the C-terminus of the Fab heavy chain to the N-terminus of the Fab heavy chain of the second antigen binding moiety. In another particular embodiment, the first and the third antigen binding moiety of the T cell activating antigen binding molecule are each fused at the C-terminus of the Fab heavy chain to the N-terminus of one of the subunits of the Fc domain, and the second antigen binding moiety is fused at the C-terminus of the Fab heavy chain to the N-terminus of the Fab heavy chain of the first antigen binding moiety. The components of the T cell activating bispecific antigen binding molecule may be fused directly or through suitable peptide linkers. In one embodiment the second and the third antigen binding moiety and the Fc domain are part of an immunoglobulin molecule. In a particular embodiment the immunoglobulin molecule is an IgG class immunoglobulin. In an even more particular embodiment the immunoglobulin is an IgG₁ subclass immunoglobulin. In another embodiment, the immunoglobulin is an IgG₄ subclass immunoglobulin.

In a particular embodiment, the Fc domain is an IgG Fc domain. In a specific embodiment, the Fc domain is an IgG₁ Fc domain. In another specific embodiment, the Fc domain is an IgG₄ Fc domain. In an even more specific embodiment, the Fc domain is an IgG₄ Fc domain comprising the amino acid substitution S228P (EU numbering). In particular embodiments the Fc domain is a human Fc domain.

In particular embodiments the Fc domain comprises a modification promoting the association of the first and the second Fc domain subunit. In a specific such embodiment, an amino acid residue in the CH3 domain of the first subunit of the Fc domain is replaced with an amino acid residue having a larger side chain volume, thereby generating a protuberance within the CH3 domain of the first subunit which is positionable in a cavity within the CH3 domain of the second subunit, and an amino acid residue in the CH3 domain of the second subunit of the Fc domain is replaced with an amino acid residue having a smaller side chain volume, thereby generating a cavity within the CH3 domain of the second subunit within which the protuberance within the CH3 domain of the first subunit is positionable.

In a particular embodiment the Fc domain exhibits reduced binding affinity to an Fc receptor and/or reduced effector function, as compared to a native IgG₁ Fc domain. In certain embodiments the Fc domain is engineered to have reduced binding affinity to an Fc receptor and/or reduced effector function, as compared to a non-engineered Fc domain. In one embodiment, the Fc domain comprises one or more amino acid substitution that reduces binding to an Fc receptor and/or effector function.

In one embodiment, the one or more amino acid substitution in the Fc domain that reduces binding to an Fc receptor and/or effector function is at one or more position selected from the group of L234, L235, and P329 (EU numbering). In particular embodiments, each subunit of the Fc domain comprises three amino acid substitutions that reduce binding to an Fc receptor and/or effector function wherein said amino acid substitutions are L234A, L235A and P329G. In one such embodiment, the Fc domain is an IgG₁ Fc domain, particularly a human IgG₁ Fc domain. In other embodiments, each subunit of the Fc domain comprises two amino acid substitutions that reduce binding to an Fc receptor and/or effector function wherein said amino acid substitutions are L235E and P329G. In one such embodiment, the Fc domain is an IgG₄ Fc domain, particularly a human IgG₄ Fc domain.

In one embodiment the Fc receptor is an Fcγ receptor. In one embodiment the Fc receptor is a human Fc receptor. In one embodiment, the Fc receptor is an activating Fc receptor. In a specific embodiment, the Fc receptor is human FcγRIIa, FcγRI, and/or FcγRIIIa. In one embodiment, the effector function is antibody-dependent cell-mediated cytotoxicity (ADCC).

In a particular embodiment, the activating T cell antigen that the bispecific antigen binding molecule is capable of binding is CD3. In other embodiments, the target cell antigen that the bispecific antigen binding molecule is capable of binding is a tumor cell antigen. In one embodiment, the target cell antigen is selected from the group consisting of: Melanoma-associated Chondroitin Sulfate Proteoglycan (MCSP), Epidermal Growth Factor Receptor (EGFR), Carcinoembryonic Antigen (CEA), Fibroblast Activation Protein (FAP), CD19, CD20 and CD33.

According to another aspect of the invention there is provided an isolated polynucleotide encoding a T cell activating bispecific antigen binding molecule of the invention or a fragment thereof. The invention also encompasses polypeptides encoded by the polynucleotides of the invention. The invention further provides an expression vector comprising the isolated polynucleotide of the invention, and a host cell comprising the isolated polynucleotide or the expression vector of the invention. In some embodiments the host cell is a eukaryotic cell, particularly a mammalian cell.

In another aspect is provided a method of producing the T cell activating bispecific antigen binding molecule of the invention, comprising the steps of a) culturing the host cell of the invention under conditions suitable for the expression of the T cell activating bispecific antigen binding molecule and b) recovering the T cell activating bispecific antigen binding molecule. The invention also encompasses a T cell activating bispecific antigen binding molecule produced by the method of the invention.

The invention further provides a pharmaceutical composition comprising the T cell activating bispecific antigen binding molecule of the invention and a pharmaceutically acceptable carrier.

Also encompassed by the invention are methods of using the T cell activating bispecific antigen binding molecule and pharmaceutical composition of the invention. In one aspect the invention provides a T cell activating bispecific antigen binding molecule or a pharmaceutical composition of the invention for use as a medicament. In one aspect is provided a T cell activating bispecific antigen binding molecule or a pharmaceutical composition according to the invention for use in the treatment of a disease in an individual in need thereof. In a specific embodiment the disease is cancer.

Also provided is the use of a T cell activating bispecific antigen binding molecule of the invention for the manufacture of a medicament for the treatment of a disease in an individual in need thereof; as well as a method of treating a disease in an individual, comprising administering to said individual a therapeutically effective amount of a composition comprising the T cell activating bispecific antigen binding molecule according to the invention in a pharmaceutically acceptable form. In a specific embodiment the disease is cancer. In any of the above embodiments the individual preferably is a mammal, particularly a human.

The invention also provides a method for inducing lysis of a target cell, particularly a tumor cell, comprising contacting a target cell with a T cell activating bispecific antigen binding molecule of the invention in the presence of a T cell, particularly a cytotoxic T cell.

### BRIEF DESCRIPTION OF THE DRAWINGS

FIG. 1A — FIG. 1M. Exemplary configurations of the T cell activating bispecific antigen binding molecules of the invention. Illustration of (FIG. 1A) the “1+1 IgG scFab, one armed”, and (FIG. 1B) the “1+1 IgG scFab, one armed inverted” molecule. In the “1+1 IgG scFab, one armed” molecule the light chain of the T cell targeting Fab is fused to the heavy chain by a linker, while the “1+1 IgG scFab, one armed inverted” molecule has the linker in the tumor targeting Fab. (FIG. 1C) Illustration of the “2+1 IgG scFab” molecule. (FIG. 1D) Illustration of the “1+1 IgG scFab” molecule. (FIG. 1E) Illustration of the “1+1 IgG Crossfab” molecule. (FIG. 1F) Illustration of the “2+1 IgG Crossfab” molecule. (FIG. 1G) Illustration of the “2+1 IgG Crossfab” molecule with alternative order of Crossfab and Fab components (“inverted”). (FIG. 1H) Illustration of the “1+1 IgG Crossfab light chain (LC) fusion” molecule. (FIG. 1I) Illustration of the “1+1 CrossMab” molecule. (FIG. 1J) Illustration of the “2+1 IgG Crossfab, linked light chain” molecule. (FIG. 1K) Illustration of the “1+1 IgG Crossfab, linked light chain” molecule. (FIG. 1L) Illustration of the “2+1 IgG Crossfab, inverted, linked light chain” molecule. (FIG. 1M) Illustration of the “1+1 IgG Crossfab, inverted, linked light chain” molecule. Black dot: optional modification in the Fc domain promoting heterodimerization.

FIG. 2A — FIG. 2D. SDS PAGE (4-12% Bis/Tris, NuPage Invitrogen, Coomassie-stained) of “1+1 IgG scFab, one armed” (anti-MCSP/anti-huCD3) (see SEQ ID NOs 1, 3, 5), non reduced (FIG. 2A) and reduced (FIG. 2B), and of “1+1 IgG scFab, one armed inverted” (anti-MCSP/anti-huCD3) (see SEQ ID NOs 7, 9, 11), non reduced (FIG. 2C) and reduced (FIG. 2D).

FIG. 3A and FIG. 3B. Analytical size exclusion chromatography (Superdex 200 10/300 GL GE Healthcare; 2 mM MOPS pH 7.3, 150 mM NaCl, 0.02% (w/v) NaCl; 50 µg sample injected) of “1+1 IgG scFab, one armed” (anti-MCSP/anti-huCD3) (see SEQ ID NOs 1, 3, 5) (FIG. 3A) and “1+1 IgG scFab, one armed inverted” (anti-MCSP/anti-huCD3) (see SEQ ID NOs 7, 9, 11) (FIG. 3B).

FIG. 4A — FIG. 4D. SDS PAGE (4-12% Bis/Tris, NuPage Invitrogen, Coomassie-stained) of “1+1 IgG scFab, one armed” (anti-EGFR/anti-huCD3) (see SEQ ID NOs 43, 45, 57), non reduced (FIG. 4A) and reduced (FIG. 4B), and of “1+1 IgG scFab, one armed inverted” (anti-EGFR/anti-huCD3) (see SEQ ID NOs 11, 49, 51), non reduced (FIG. 4C) and reduced (FIG. 4D).

FIG. 5A and FIG. 5B. Analytical size exclusion chromatography (Superdex 200 10/300 GL GE Healthcare; 2 mM MOPS pH 7.3, 150 mM NaCl, 0.02% (w/v) NaCl; 50 µg sample injected) of “1+1 IgG scFab, one armed” (anti-EGFR/anti-huCD3) (see SEQ ID NOs 43, 45, 47) (FIG. 5A) and “1+1 IgG scFab, one armed inverted” (anti-EGFR/anti-huCD3) (see SEQ ID NOs 11, 49, 51) (FIG. 5B).

FIG. 6A — FIG. 6C. (FIG. 6A, FIG. 6B) SDS PAGE (4-12% Bis/Tris, NuPage Invitrogen, Coomassie-stained) of “1+1 IgG scFab, one armed inverted” (anti-FAP/anti-huCD3) (see SEQ ID NOs 11, 51, 55), non reduced (FIG. 6A) and reduced (FIG. 6B). (FIG. 6C) Analytical size exclusion chromatography (Superdex 200 10/300 GL GE Healthcare; 2 mM MOPS pH 7.3, 150 mM NaCl, 0.02% (w/v) NaCl; 50 µg sample injected) of “1+1 IgG scFab, one armed inverted” (anti-FAP/anti-huCD3).

FIG. 7A — FIG. 7D. SDS PAGE (4-12% Bis/Tris, NuPage Invitrogen, Coomassie-stained) of (FIG. 7A) “2+1 IgG scFab, P329G LALA” (anti-MCSP/anti-huCD3) (see SEQ ID NOs 5, 21, 23), non reduced (lane 2) and reduced (lane 3); of (FIG. 7B) “2+1 IgG scFab, LALA” (anti-MCSP/anti-huCD3) (see SEQ ID NOs 5, 17, 19), non reduced (lane 2) and reduced (lane 3); of (FIG. 7C) “2+1 IgG scFab, wt” (anti-MCSP/anti-huCD3) (see SEQ ID NOs 5, 13, 15), non reduced (lane 2) and reduced (lane 3); and of (FIG. 7D) “2+1 IgG scFab, P329G LALA N297D” (anti-MCSP/anti-huCD3) (see SEQ ID NOs 5, 25, 27), non reduced (lane 2) and reduced (lane 3).

FIG. 8A — FIG. 8D. Analytical size exclusion chromatography (Superdex 200 10/300 GL GE Healthcare; 2 mM MOPS pH 7.3, 150 mM NaCl, 0.02% (w/v) NaCl; 50 µg sample injected) of (FIG. 8A) “2+1 IgG scFab, P329G LALA” (anti-MCSP/anti-huCD3) (see SEQ ID NOs 5, 21, 23); of (FIG. 8B) “2+1 IgG scFab, LALA” (anti-MCSP/anti-huCD3) (see SEQ ID NOs 5, 17, 19); of (FIG. 8C) “2+1 IgG scFab, wt” (anti-MCSP/anti-huCD3) (see SEQ ID NOs 5, 13, 15); and of (FIG. 8D) “2+1 IgG scFab, P329G LALA N297D” (anti-MCSP/anti-huCD3) (see SEQ ID NOs 5, 25, 27).

FIG. 9A — FIG. 9C. (FIG. 9A, FIG. 9B) SDS PAGE (4-12% Bis/Tris, NuPage Invitrogen, Coomassie-stained) of “2+1 IgG scFab, P329G LALA” (anti-EGFR/anti-huCD3) (see SEQ ID NOs 45, 47, 53), non reduced (FIG. 9A) and reduced (FIG. 9B). (FIG. 9C) Analytical size exclusion chromatography (Superdex 200 10/300 GL GE Healthcare; 2 mM MOPS pH 7.3, 150 mM NaCl, 0.02% (w/v) NaCl; 50 µg sample injected) of “2+1 IgG scFab, P329G LALA” (anti-EGFR/anti-huCD3).

FIG. 10A — FIG. 10C. (FIG. 10A, FIG. 10B) SDS PAGE (4-12% Bis/Tris, NuPage Invitrogen, Coomassie-stained) of “2+1 IgG scFab, P329G LALA” (anti-FAP/anti-huCD3) (see SEQ ID NOs 57, 59, 61), non reduced (FIG. 10A) and reduced (FIG. 10B). (FIG. 10C) Analytical size exclusion chromatography (Superdex 200 10/300 GL GE Healthcare; 2 mM MOPS pH 7.3, 150 mM NaCl, 0.02% (w/v) NaCl; 50 µg sample injected) of “2+1 IgG scFab, P329G LALA” (anti-FAP/anti-huCD3).

FIG. 11A - FIG. 11C. (FIG. 11A, FIG. 11B) SDS PAGE (4-12% Tris-Acetate (FIG. 11A) or 4-12% Bis/Tris (FIG. 11B), NuPage Invitrogen, Coomassie-stained) of “1+1 IgG Crossfab, Fc(hole) P329G LALA / Fc(knob) wt” (anti-MCSP/anti-huCD3) (see SEQ ID NOs 5, 29, 31, 33), non reduced (FIG. 11A) and reduced (FIG. 11B). (FIG. 11C) Analytical size exclusion chromatography (Superdex 200 10/300 GL GE Healthcare; 2 mM MOPS pH 7.3, 150 mM NaCl, 0.02% (w/v) NaCl; 50 µg sample injected) of “1+1 IgG Crossfab, Fc(hole) P329G LALA / Fc(knob) wt” (anti-MCSP/anti-huCD3).

FIG. 12A - FIG. 12C. (FIG. 12A, FIG. 12B) SDS PAGE (4-12% Bis/Tris, NuPage Invitrogen, Coomassie-stained) of “2+1 IgG Crossfab” (anti-MCSP/anti-huCD3) (see SEQ ID NOs 3, 5, 29, 33), non reduced (FIG. 12A) and reduced (FIG. 12B). (FIG. 12C) Analytical size exclusion chromatography (Superdex 200 10/300 GL GE Healthcare; 2 mM MOPS pH 7.3, 150 mM NaCl, 0.02% (w/v) NaCl; 50 µg sample injected) of “2+1 IgG Crossfab” (anti-MCSP/anti-huCD3).

FIG. 13A - FIG. 13C. (FIG. 13A, FIG. 13B) SDS PAGE (4-12% Bis/Tris, NuPage Invitrogen, Coomassie-stained) of “2+1 IgG Crossfab” (anti-MCSP/anti-cyCD3) (see SEQ ID NOs 3, 5, 35, 37), non reduced (FIG. 13A) and reduced (FIG. 13B). (FIG. 13C) Analytical size exclusion chromatography (Superdex 200 10/300 GL GE Healthcare; 2 mM MOPS pH 7.3, 150 mM NaCl, 0.02% (w/v) NaCl; 50 µg sample injected) of “2+1 IgG Crossfab” (anti-MCSP/anti-cyCD3).

FIG. 14A - FIG. 14C. (FIG. 14A, FIG. 14B) SDS PAGE (4-12% Bis/Tris, NuPage Invitrogen, Coomassie-stained) of “2+1 IgG Crossfab, inverted” (anti-CEA/anti-huCD3) (see SEQ ID NOs 33, 63, 65, 67), non reduced (FIG. 14A) and reduced (FIG. 14B). (FIG. 14C) Analytical size exclusion chromatography (Superdex 200 10/300 GL GE Healthcare; 2 mM MOPS pH 7.3, 150 mM NaCl, 0.02% (w/v) NaCl; 50 µg sample injected) of “2+1 IgG Crossfab, inverted” (anti-CEA/anti-huCD3).

FIG. 15A and FIG. 15B. (FIG. 15A) Thermal stability of “(scFv)₂-Fc” and “(dsscFv)₂-Fc” (anti-MCSP (LC007)/anti-huCD3 (V9)). Dynamic Light Scattering, measured in a temperature ramp from 25-75° C. at 0.05° C./min. Black curve: “(scFv)₂-Fc”; grey curve: “(dsscFv)₂-Fc”. (FIG. 15B) Thermal stability of “2+1 IgG scFab” (see SEQ ID NOs 5, 21, 23) and “2+1 IgG Crossfab” (anti-MCSP/anti-huCD3) (see SEQ ID NOs 3, 5, 29, 33). Dynamic Light Scattering, measured in a temperature ramp from 25-75° C. at 0.05° C./min. Black curve: “2+1 IgG scFab”; grey curve: “2+1 IgG Crossfab”.

FIG. 16A and FIG. 16B. Biacore assay setup for (FIG. 16A) determination of interaction of various Fc-mutants with human FcγRIIIa, and for (FIG. 16B) simultaneous binding of T cell bespecific constructs with tumor target and human CD3γ(G₄S)₅CD3ε-AcTev-Fc(knob)-Avi/Fc(hole).

FIG. 17A and FIG. 17B. Simultaneous binding of T-cell bispecific constructs to the D3 domain of human MCSP and human CD3γ(G₄S)₅CD3ε-AcTev-Fc(knob)-Avi/Fc(hole). (FIG. 17A) “2+1 IgG Crossfab” (see SEQ ID NOs 3, 5, 29, 33), (FIG. 17B) “2+1 IgG scFab” (see SEQ ID NOs 5, 21, 23).

FIG. 18A — FIG. 18D. Simultaneous binding of T-cell bispecific constructs to human EGFR and human CD3γ(G₄S)₅CD3ε-AcTev-Fc(knob)-Avi/Fc(hole). (FIG. 18A) “2+1 IgG scFab” (see SEQ ID NOs 45, 47, 53), (FIG. 18B) “1+1 IgG scFab, one armed” (see SEQ ID NOs 43, 45, 47), (FIG. 18C) “1+1 IgG scFab, one armed inverted” (see SEQ ID NOs 11, 49, 51), and (FIG. 18D) “1+1 IgG scFab” (see SEQ ID NOs 47, 53, 213).

FIG. 19A and FIG. 19B. Binding of the “(scFv)₂” molecule (50 nM) to CD3 expressed on Jurkat cells (FIG. 19A), or to MCSP on Colo-38 cells (FIG. 19B) measured by FACS. Mean fluorescence intensity compared to untreated cells and cells stained with the secondary antibody only is depicted.

FIG. 20A and FIG. 20B. Binding of the “2+1 IgG scFab, LALA” (see SEQ ID NOs 5, 17, 19) construct (50 nM) to CD3 expressed on Jurkat cells (FIG. 20A), or to MCSP on Colo-38 cells (FIG. 20B) measured by FACS. Mean fluorescence intensity compared to cells treated with the reference anti-CD3 IgG (as indicated), untreated cells, and cells stained with the secondary antibody only is depicted.

FIG. 21A and FIG. 21B. Binding of the “1+1 IgG scFab, one armed” (see SEQ ID NOs 1, 3, 5) and “1+1 IgG scFab, one armed inverted” (see SEQ ID NOs 7, 9, 11) constructs (50 nM) to CD3 expressed on Jurkat cells (FIG. 21A), or to MCSP on Colo-38 cells (FIG. 21B) measured by FACS. Mean fluorescence intensity compared to cells treated with the reference anti-CD3 or anti-MCSP IgG (as indicated), untreated cells, and cells stained with the secondary antibody only is depicted.

FIG. 22. Dose dependent binding of the “2+1 IgG scFab, LALA” (see SEQ ID NOs 5, 17, 19) bispecific construct and the corresponding anti-MCSP IgG to MCSP on Colo-38 cells as measured by FACS.

FIG. 23A and FIG. 23B. Surface expression level of different activation markers on human T cells after incubation with 1 nM of “2+1 IgG scFab, LALA” (see SEQ ID NOs 5, 17, 19) or “(scFv)₂” CD3-MCSP bispecific constructs in the presence or absence of Colo-38 tumor target cells, as indicated (E:T ratio of PBMCs to tumor cells = 10:1). Depicted is the expression level of the early activation marker CD69 (FIG. 23A), or the late activation marker CD25 (FIG. 23B) on CD8⁺ T cells after 15 or 24 hours incubation, respectively.

FIG. 24A and FIG. 24B. Surface expression level of the late activation marker CD25 on human T cells after incubation with 1 nM of “2+1 IgG scFab, LALA” (see SEQ ID NOs 5, 17, 19) or “(scFv)₂” CD3-MCSP bispecific constructs in the presence or absence of Colo-38 tumor target cells, as indicated (E:T ratio = 5:1). Depicted is the expression level of the late activation marker CD25 on CD8⁺ T cells (FIG. 24A) or on CD4⁺ T cells (FIG. 24B) after 5 days incubation.

FIG. 25. Surface expression level of the late activation marker CD25 on cynomolgus CD8⁺ T cells from two different animals (cyno Nestor, cyno Nobu) after 43 hours incubation with the indicated concentrations of the “2+1 IgG Crossfab” bispecific construct (targeting cynomolgus CD3 and human MCSP; see SEQ ID NOs 3, 5, 35, 37), in the presence or absence of human MCSP-expressing MV-3 tumor target cells (E:T ratio = 3:1). As controls, the reference IgGs (anti-cynomolgus CD3 IgG, anti-human MCSP IgG) or the unphysiologic stimulus PHA-M were used.

FIG. 26. IFN-γ levels, secreted by human pan T cells that were activated for 18.5 hours by the “2+1 IgG scFab, LALA” CD3-MCSP bispecific construct (see SEQ ID NOs 5, 17, 19) in the presence of U87MG tumor cells (E:T ratio = 5:1). As controls, the corresponding anti-CD3 and anti-MCSP IgGs were administered.

FIG. 27. Killing (as measured by LDH release) of MDA-MB-435 tumor cells upon co-culture with human pan T cells (E:T ratio = 5:1) and activation for 20 hours by different concentrations of the “2+1 IgG scFab” (see SEQ ID NOs 5, 21, 23), “2+1 IgG Crossfab” (see SEQ ID NOs 3, 5, 29, 33) and “(scFv)₂” bispecific molecules and corresponding IgGs.

FIG. 28. Killing (as measured by LDH release) of MDA-MB-435 tumor cells upon co-culture with human pan T cells (E:T ratio = 5:1), and activation for 20 hours by different concentrations of the bispecific constructs and corresponding IgGs. “2+1 IgG scFab” constructs differing in their Fc-domain (having either a wild-type Fc domain (see SEQ ID NOs 5, 13, 15), or a Fc-domain mutated to abolish (NK) effector cell function: P329G LALA (see SEQ ID NOs 5, 21, 23), P329G LALA N297D (see SEQ ID NOs 5, 25, 27)) and the “2+1 IgG Crossfab” (see SEQ ID NOs 3, 5, 29, 33) construct were compared.

FIG. 29. Killing (as measured by LDH release) of Colo-38 tumor cells upon co-culture with human pan T cells (E:T ratio = 5:1), treated with CD3-MCSP bispecific “2+1 IgG scFab, LALA” (see SEQ ID NOs 5, 17, 19) construct, “(scFv)₂” molecule or corresponding IgGs for 18.5 hours.

FIG. 30. Killing (as measured by LDH release) of Colo-38 tumor cells upon co-culture with human pan T cells (E:T ratio = 5:1), treated with CD3-MCSP bispecific “2+1 IgG scFab, LALA” (see SEQ ID NOs 5, 17, 19) construct, the “(scFv)₂” molecule or corresponding IgGs for 18 hours.

FIG. 31. Killing (as measured by LDH release) of MDA-MB-435 tumor cells upon co-culture with human pan T cells (E:T ratio = 5:1), and activation for 23.5 hours by different concentrations of the CD3-MCSP bispecific “2+1 IgG scFab, LALA” (see SEQ ID NOs 5, 17, 19) construct, “(scFv)₂” molecule or corresponding IgGs.

FIG. 32. Killing (as measured by LDH release) of Colo-38 tumor cells upon co-culture with human pan T cells (E:T ratio = 5:1) and activation for 19 hours by different concentrations of the CD3-MCSP bispecific “1+1 IgG scFab, one armed” (see SEQ ID NOs 1, 3, 5), “1+1 IgG scFab, one armed inverted” (see SEQ ID NOs 7, 9, 11) or “(scFv)₂” constructs, or corresponding IgGs.

FIG. 33. Killing (as measured by LDH release) of Colo-38 tumor cells upon co-culture with human pan T cells (E:T ratio = 5:1), treated with “1+1 IgG scFab” CD3-MCSP bispecific construct (see SEQ ID NOs 5, 21, 213) or “(scFv)₂” molecule for 20 hours.

FIG. 34. Killing (as measured by LDH release) of MDA-MB-435 tumor cells upon co-culture with human pan T cells (E:T ratio = 5:1), and activation for 21 hours by different concentrations of the bispecific constructs and corresponding IgGs. The CD3-MCSP bispecific “2+1 IgG Crossfab” (see SEQ ID NOs 3, 5, 29, 33) and “1+1 IgG Crossfab” (see SEQ ID NOs 5, 29, 31, 33) constructs, the “(scFv)₂” molecule and corresponding IgGs were compared.

FIG. 35. Killing (as measured by LDH release) of different target cells (MCSP-positive Colo-38 tumor target cells, mesenchymal stem cells derived from bone marrow or adipose tissue, or pericytes from placenta; as indicated) induced by the activation of human T cells by 135 ng/ml or 1.35 ng/ml of the “2+1 IgG Crossfab” CD3-MCSP bispecific construct (see SEQ ID NOs 3, 5, 29, 33) (E:T ratio = 25:1).

FIG. 36A and FIG. 36B. Killing (as measured by LDH release) of Colo-38 tumor target cells, measured after an overnight incubation of 21 h, upon co-culture with human PBMCs and different CD3-MCSP bispecific constructs (“2+1 IgG scFab, LALA” (see SEQ ID NOs 5, 17, 19) and “(scFv)₂”) or a glycoengineered anti-MCSP IgG (GlycoMab). The effector to target cell ratio was fixed at 25:1 (FIG. 36A), or varied as depicted (FIG. 36B). PBMCs were isolated from fresh blood (FIG. 36A) or from a Buffy Coat (FIG. 36B).

FIG. 37. Time-dependent cytotoxic effect of the “2+1 IgG Crossfab” construct, targeting cynomolgus CD3 and human MCSP (see SEQ ID NOs 3, 5, 35, 37). Depicted is the LDH release from human MCSP-expressing MV-3 cells upon co-culture with primary cynomolgus PBMCs (E:T ratio = 3:1) for 24 h or 43 h. As controls, the reference IgGs (anti-cyno CD3 IgG and anti-human MCSP IgG) were used at the same molarity. PHA-M served as a control for (unphysiologic) T cell activation.

FIG. 38. Killing (as measured by LDH release) of huMCSP-positive MV-3 melanoma cells upon co-culture with human PBMCs (E:T ratio = 10:1), treated with different CD3-MCSP bispecific constructs (“2+1 IgG Crossfab” (see SEQ ID NOs 3, 5, 29, 33) and “(scFv)₂”) for ~26 hours.

FIG. 39. Killing (as measured by LDH release) of EGFR-positive LS-174T tumor cells upon co-culture with human pan T cells (E:T ratio = 5:1), treated with different CD3-EGFR bispecific constructs (“2+1 IgG scFab” (see SEQ ID NOs 45, 47, 53), “1+1 IgG scFab” (see SEQ ID NOs 47, 53, 213) and “(scFv)₂”) or reference IgGs for 18 hours.

FIG. 40. Killing (as measured by LDH release) of EGFR-positive LS-174T tumor cells upon co-culture with human pan T cells (E:T ratio = 5:1), treated with different CD3-EGFR bispecific constructs (“1+1 IgG scFab, one armed” (see SEQ ID NOs 43, 45, 47), “1+1 IgG scFab, one armed inverted” (see SEQ ID NOs 11, 49, 51), “1+1 IgG scFab” (see SEQ ID NOs 47, 53, 213) and “(scFv)₂”) or reference IgGs for 21 hours.

FIG. 41A and FIG. 41B. Killing (as measured by LDH release) of EGFR-positive LS-174T tumor cells upon co-culture with either human pan T cells (FIG. 41A) or human naive T cells (FIG. 41B), treated with different CD3-EGFR bispecific constructs (“1+1 IgG scFab, one armed” (see SEQ ID NOs 43, 45, 47), “1+1 IgG scFab, one armed inverted” (see SEQ ID NOs 11, 49, 51) and “(scFv)₂”) or reference IgGs for 16 hours. The effector to target cell ratio was 5:1.

FIG. 42. Killing (as measured by LDH release) of FAP-positive GM05389 fibroblasts upon co-culture with human pan T cells (E:T ratio = 5:1), treated with different CD3-FAP bispecific constructs (“1+1 IgG scFab, one armed inverted” (see SEQ ID NOs 11, 51, 55), “1+1 IgG scFab” (see SEQ ID NOs 57, 61, 213), “2+1 IgG scFab” (see SEQ ID NOs 57, 59, 61) and “(scFv)₂”) for ~18 hours.

FIG. 43A and FIG. 43B. Flow cytrometric analysis of expression levels of CD107a/b, as well as perforin levels in CD8⁺ T cells that have been treated with different CD3-MCSP bispecific constructs (“2+1 IgG scFab, LALA” (see SEQ ID NOs 5, 17, 19) and “(scFv)₂”) or corresponding control IgGs in the presence (FIG. 43A) or absence (FIG. 43B) of target cells for 6 h. Human pan T cells were incubated with 9.43 nM of the different molecules in the presence or absence of Colo-38 tumor target cells at an effector to target ratio of 5:1. Monensin was added after the first hour of incubation to increase intracellular protein levels by preventing protein transport. Gates were set either on all CD107a/b positive, perforin-positive or double-positive cells, as depicted.

FIG. 44A and FIG. 44B. Relative proliferation of either CD8⁺ (FIG. 44A) or CD4⁺ (FIG. 44B) human T cells upon incubation with 1 nM of different CD3-MCSP bispecific constructs (“2+1 IgG scFab, LALA” (see SEQ ID NOs 5, 17, 19) or “(scFv)₂”) or corresponding control IgGs in the presence or absence of Colo-38 tumor target cells at an effector to target cell ratio of 5:1. CFSE-labeled human pan T cells were characterized by FACS. The relative proliferation level was determined by setting a gate around the non-proliferating cells and using the cell number of this gate relative to the overall measured cell number as the reference.

FIG. 45A and FIG. 45B. Levels of different cytokines measured in the supernatant of human PBMCs after treatment with 1 nM of different CD3-MCSP bispecific constructs (“2+1 IgG scFab, LALA” (see SEQ ID NOs 5, 17, 19) or “(scFv)₂”) or corresponding control IgGs in the presence (FIG. 45A) or absence (FIG. 45B) of Colo-38 tumor cells for 24 hours. The effector to target cell ratio was 10:1.

FIG. 46A - FIG. 46D. Levels of different cytokines measured in the supernatant of whole blood after treatment with 1 nM of different CD3-MCSP bispecific constructs (“2+1 IgG scFab”, “2+1 IgG Crossfab” (see SEQ ID NOs 3, 5, 29, 33) or “(scFv)₂”) or corresponding control IgGs in the presence (FIG. 46A, FIG. 46B) or absence (FIG. 46C, FIG. 46D) of Colo-38 tumor cells for 24 hours. Among the bispecific constructs were different “2+1 IgG scFab” constructs having either a wild-type Fc domain (see SEQ ID NOs 5, 13, 15), or an Fc domain mutated to abolish (NK) effector cell function (LALA (see SEQ ID NOs 5, 17, 19), P329G LALA (see SEQ ID NOs 5, 2, 23) and P329G LALA N297D (see SEQ ID NOs 5, 25, 27)).

FIG. 47. CE-SDS analyses. Electropherogram shown as SDS PAGE of 2+1 IgG Crossfab, linked light chain (see SEQ ID NOs 3, 5, 29, 179). (lane 1: reduced, lane 2: non-reduced).

FIG. 48. Analytical size exclusion chromatography of 2+1 IgG Crossfab, linked light chain (see SEQ ID NOs 3, 5, 29, 179) (final product). 20 µg sample were injected.

FIG. 49. Killing (as measured by LDH release) of MCSP-positive MV-3 tumor cells upon co-culture by human PBMCs (E:T ratio = 10:1), treated with different CD3-MCSP bispecific constructs for ~ 44 hours (“2+1 IgG Crossfab” (see SEQ ID NOs 3, 5, 29, 33) and “2+1 IgG Crossfab, linked LC” (see SEQ ID NOs 3, 5, 29, 179)). Human PBMCs were isolated from fresh blood of healthy volunteers.

FIG. 50. Killing (as measured by LDH release) of MCSP-positive Colo-38 tumor cells upon co-culture by human PBMCs (E:T ratio = 10:1), treated with different CD3-MCSP bispecific constructs for ~22 hours (“2+1 IgG Crossfab” (see SEQ ID NOs 3, 5, 29, 33) and “2+1 IgG Crossfab, linked LC” (see SEQ ID NOs 3, 5, 29, 179)). Human PBMCs were isolated from fresh blood of healthy volunteers.

FIG. 51. Killing (as measured by LDH release) of MCSP-positive Colo-38 tumor cells upon co-culture by human PBMCs (E:T ratio = 10:1), treated with different CD3-MCSP bispecific constructs for ~22 hours (“2+1 IgG Crossfab” (see SEQ ID NOs 3, 5, 29, 33) and “2+1 IgG Crossfab, linked LC” (see SEQ ID NOs 3, 5, 29, 179)). Human PBMCs were isolated from fresh blood of healthy volunteers.

FIG. 52. Killing (as measured by LDH release) of MCSP-positive WM266-4 cells upon co-culture by human PBMCs (E:T ratio = 10:1), treated with different CD3-MCSP bispecific constructs for ~22 hours (“2+1 IgG Crossfab” (see SEQ ID NOs 3, 5, 29, 33) and “2+1 IgG Crossfab, linked LC” (see SEQ ID NOs 3, 5, 29, 179)). Human PBMCs were isolated from fresh blood of healthy volunteers.

FIG. 53A and FIG. 53B. Surface expression level of the early activation marker CD69 (FIG. 53A) and the late activation marker CD25 (FIG. 53B) on human CD8⁺ T cells after 22 hours incubation with 10 nM, 80 pM or 3 pM of different CD3-MCSP bispecific constructs (“2+1 IgG Crossfab” (see SEQ ID NOs 3, 5, 29, 33) and “2+1 IgG Crossfab, linked LC” (see SEQ ID NOs 3, 5, 29, 179)) in the presence or absence of human MCSP-expressing Colo-38 tumor target cells (E:T ratio = 10:1).

FIG. 54A — FIG. 54N. CE-SDS analyses. (FIG. 54A) Electropherogram shown as SDS-PAGE of 1+1 IgG Crossfab; VL/VH exchange (LC007/V9) (see SEQ ID NOs 5, 29, 33, 181): a) non-reduced, b) reduced. (FIG. 54B) Electropherogram shown as SDS-PAGE of 1+1 CrossMab; CL/CH1 exchange (LC007/V9) (see SEQ ID NOs 5, 23, 183, 185): a) reduced, b) non-reduced. (FIG. 54C) Electropherogram shown as SDS-PAGE of 2+1 IgG Crossfab, inverted; CL/CH1 exchange (LC007/V9) (see SEQ ID NOs 5, 23, 183, 187): a) reduced, b) non-reduced. (FIG. 54D) Electropherogram shown as SDS-PAGE of 2+1 IgG Crossfab; VL/VH exchange (M4-3 ML2/V9) (see SEQ ID NOs 33, 189, 191, 193): a) reduced, b) non-reduced. (FIG. 54E) Electropherogram shown as SDS-PAGE of 2+1 IgG Crossfab; CL/CH1 exchange (M4-3 ML2/V9) (see SEQ ID NOs 183, 189, 193, 195): a) reduced, b) non-reduced. (FIG. 54F) Electropherogram shown as SDS-PAGE of 2+1 IgG Crossfab, inverted; CL/CH1 exchange (CH1A1A/V9) (see SEQ ID NOs 65, 67, 183, 197): a) reduced, b) non-reduced. (FIG. 54G) Electropherogram shown as SDS-PAGE of 2+1 IgG Crossfab; CL/CH1 exchange (M4-3 ML2/H2C) (see SEQ ID NOs 189, 193, 199, 201): a) reduced, b) non-reduced. (FIG. 54H) Electropherogram shown as SDS-PAGE of 2+1 IgG Crossfab, inverted; CL/CH1 exchange (431/26/V9) (see SEQ ID NOs 183, 203, 205, 207): a) reduced, b) non-reduced. (FIG. 54I) Electropherogram shown as SDS-PAGE of “2+1 IgG Crossfab light chain fusion” (CH1A1A/V9) (see SEQ ID NOs 183, 209, 211, 213): a) reduced, b) non-reduced. (FIG. 54J) SDS PAGE (4-12% Bis/Tris, NuPage Invitrogen, Coomassie-stained) of “2+1 IgG Crossfab” (anti-MCSP/anti-huCD3) (see SEQ ID NOs 5, 23, 215, 217), non-reduced (left) and reduced (right). (FIG. 54K) Electropherogram shown as SDS-PAGE of “2+1 IgG Crossfab, inverted” (anti-MCSP/anti-huCD3) (see SEQ ID NOs 5, 23, 215, 219): a) reduced, b) non-reduced. (FIG. 54L) SDS PAGE (4-12% Bis/Tris, NuPage Invitrogen, Coomassie-stained) of “1+1 IgG Crossfab” (anti-CD33/anti-huCD3) (see SEQ ID NOs 33, 213, 221, 223), reduced (left) and non-reduced (right). (FIG. 54M) SDS PAGE (4-12% Bis/Tris, NuPage Invitrogen, Coomassie-stained) of “2+1 IgG Crossfab” (anti-CD33/anti-huCD3) (see SEQ ID NOs 33, 221, 223, 225), reduced (left) and non-reduced (right). (FIG. 54N) SDS PAGE (4-12% Bis/Tris, NuPage Invitrogen, Coomassie-stained) of “2+1 IgG Crossfab” (anti-CD20/anti-huCD3) (see SEQ ID NOs 33, 227, 229, 231), non-reduced.

FIG. 55A and FIG. 55B. Binding of bispecific constructs (CEA/CD3 “2+1 IgG Crossfab, inverted (VL/VH)” (see SEQ ID NOs 33, 63, 65, 67) and “2+1 IgG Crossfab, inverted (CL/CH1)” 2 (see SEQ ID NOs 65, 67, 183, 197)) to human CD3, expressed by Jurkat cells (FIG. 55A), or to human CEA, expressed by LS-174T cells (FIG. 55B) as determined by FACS. As a control, the equivalent maximum concentration of the reference IgGs and the background staining due to the labeled 2ndary antibody (goat anti-human FITC-conjugated AffiniPure F(ab′)2 Fragment, Fcγ Fragment-specific, Jackson Immuno Research Lab # 109-096-098) were assessed as well.

FIG. 56A and FIG. 56B. Binding of bispecific constructs constructs (MCSP/CD3 “2+1 IgG Crossfab” (see SEQ ID NOs 3, 5, 29, 33) and “2+1 IgG Crossfab, inverted” (see SEQ ID NOs 5, 23, 183, 187)) to human CD3, expressed by Jurkat cells (FIG. 56A), or to human MCSP, expressed by WM266-4 tumor cells (FIG. 56B) as determined by FACS.

FIG. 57A and FIG. 57B. Binding of the “1+1 IgG Crossfab light chain fusion” (see SEQ ID NOs 183, 209, 211, 213) to human CD3, expressed by Jurkat cells (FIG. 57A), or to human CEA, expressed by LS-174T cells (FIG. 57B) as determined by FACS.

FIG. 58A and FIG. 58B. Binding of the “2+1 IgG Crossfab” (see SEQ ID NOs 5, 23, 215, 217) and the “2+1 IgG Crossfab, inverted” (see SEQ ID NOs 5, 23, 215, 219) constructs to human CD3, expressed by Jurkat cells (FIG. 58A), or human MCSP, expressed by WM266-4 tumor cells (FIG. 58B) as determined by FACS.

FIG. 59A and FIG. 59B. Surface expression level of the early activation marker CD69 (FIG. 59A) or the late activation marker CD25 (FIG. 59B) on human CD4⁺ or CD8⁺ T cells after 24 hours incubation with the indicated concentrations of the CD3/MCSP “1+1 CrossMab” (see SEQ ID NOs 5, 23, 183, 185), “1+1 IgG Crossfab” (see SEQ ID NOs 5, 29, 33, 181) and “2+1 IgG Crossfab” (see SEQ ID NOs 3, 5, 29, 33) constructs. The assay was performed in the presence or absence of MV-3 target cells, as indicated.

FIG. 60A and FIG. 60B. Surface expression level of the early activation marker CD25 on CD4⁺ or CD8⁺ T cells from two different cynomolgus monkeys (FIG. 60A and FIG. 60B) in the presence or absence of huMCSP-positive MV-3 tumor cells upon co-culture with cynomolgus PBMCs (E:T ratio = 3:1, normalized to CD3⁺ numbers), treated with the “2+1 IgG Crossfab” (see SEQ ID NOs 5, 23, 215, 217) and the “2+1 IgG Crossfab, inverted” (see SEQ ID NOs 5, 23, 215, 219) for ~41 hours.

FIG. 61A and FIG. 61B. Killing (as measured by LDH release) of MKN-45 (FIG. 61A) or LS-174T (FIG. 61B) tumor cells upon co-culture with human PBMCs (E:T ratio = 10:1) and activation for 28 hours by different concentrations of the “2+1 IgG Crossfab, inverted (VL/VH)” (see SEQ ID NOs 33, 63, 65, 67) versus the “2+1 IgG Crossfab, inverted (CL/CH1)” (see SEQ ID NOs 65, 67, 183, 197) construct.

FIG. 62. Killing (as measured by LDH release) of WM266-4 tumor cells upon co-culture with human PBMCs (E:T ratio = 10:1) and activation for 26 hours by different concentrations of the “2+1 IgG Crossfab (VL/VH)” (see SEQ ID NOs 33, 189, 191, 193) versus the “2+1 IgG Crossfab (CL/CH1)” (see SEQ ID NOs 183, 189, 193, 195) construct.

FIG. 63. Killing (as measured by LDH release) of MV-3 tumor cells upon co-culture with human PBMCs (E:T ratio = 10:1) and activation for 27 hours by different concentrations of the “2+1 IgG Crossfab (VH/VL)” (see SEQ ID NOs 33, 189, 191, 193) versus the “2+1 IgG Crossfab (CL/CH1)” (see SEQ ID NOs 183, 189, 193, 195) constructs.

FIG. 64A and FIG. 64B. Killing (as measured by LDH release) of human MCSP-positive WM266-4 (FIG. 64A) or MV-3 (FIG. 64B) tumor cells upon co-culture with human PBMCs (E:T ratio = 10:1) and activation for 21 hours by different concentrations of the “2+1 IgG Crossfab” (see SEQ ID NOs 3, 5, 29, 33), the “1+1 CrossMab” (see SEQ ID NOs 5, 23, 183, 185), and the “1+1 IgG Crossfab” (see SEQ ID NOs 5, 29, 33, 181), as indicated.

FIG. 65A and FIG. 65B. Killing (as measured by LDH release) of MKN-45 (FIG. 65A) or LS-174T (FIG. 65B) tumor cells upon co-culture with human PBMCs (E:T ratio = 10:1) and activation for 28 hours by different concentrations of the “1+1 IgG Crossfab LC fusion” (see SEQ ID NOs 183, 209, 211, 213).

FIG. 66. Killing (as measured by LDH release) of MC38-huCEA tumor cells upon co-culture with human PBMCs (E:T ratio = 10:1) and activation for 24 hours by different concentrations of the “1+1 IgG Crossfab LC fusion” (see SEQ ID NOs 183, 209, 211, 213) versus an untargeted “2+1 IgG Crossfab” reference.

FIG. 67A and FIG. 67B. Killing (as measured by LDH release) of human MCSP-positive MV-3 (FIG. 67A) or WM266-4 (FIG. 67B) tumor cells upon co-culture with human PBMCs (E:T ratio = 10:1), treated with the “2+1 IgG Crossfab (V9)” (see SEQ ID NOs 3, 5, 29, 33) and the “2+1 IgG Crossfab, inverted (V9)” (see SEQ ID NOs 5, 23, 183, 187), the “2+1 IgG Crossfab (anti-CD3)” (see SEQ ID NOs 5, 23, 215, 217) and the “2+1 IgG Crossfab, inverted (anti-CD3)” (see SEQ ID NOs 5, 23, 215, 219) constructs.

### DETAILED DESCRIPTION OF THE INVENTION

### Definitions

Terms are used herein as generally used in the art, unless otherwise defined in the following.

As used herein, the term “antigen binding molecule” refers in its broadest sense to a molecule that specifically binds an antigenic determinant. Examples of antigen binding molecules are immunoglobulins and derivatives, e.g. fragments, thereof.

The term “bispecific” means that the antigen binding molecule is able to specifically bind to at least two distinct antigenic determinants. Typically, a bispecific antigen binding molecule comprises two antigen binding sites, each of which is specific for a different antigenic determinant. In certain embodiments the bispecific antigen binding molecule is capable of simultaneously binding two antigenic determinants, particularly two antigenic determinants expressed on two distinct cells.

The term “valent” as used herein denotes the presence of a specified number of antigen binding sites in an antigen binding molecule. As such, the term “monovalent binding to an antigen” denotes the presence of one (and not more than one) antigen binding site specific for the antigen in the antigen binding molecule.

An “antigen binding site” refers to the site, i.e. one or more amino acid residues, of an antigen binding molecule which provides interaction with the antigen. For example, the antigen binding site of an antibody comprises amino acid residues from the complementarity determining regions (CDRs). A native immunoglobulin molecule typically has two antigen binding sites, a Fab molecule typically has a single antigen binding site.

As used herein, the term “antigen binding moiety” refers to a polypeptide molecule that specifically binds to an antigenic determinant. In one embodiment, an antigen binding moiety is able to direct the entity to which it is attached (e.g. a second antigen binding moiety) to a target site, for example to a specific type of tumor cell or tumor stroma bearing the antigenic determinant. In another embodiment an antigen binding moiety is able to activate signaling through its target antigen, for example a T cell receptor complex antigen. Antigen binding moieties include antibodies and fragments thereof as further defined herein. Particular antigen binding moieties include an antigen binding domain of an antibody, comprising an antibody heavy chain variable region and an antibody light chain variable region. In certain embodiments, the antigen binding moieties may comprise antibody constant regions as further defined herein and known in the art. Useful heavy chain constant regions include any of the five isotypes: α, δ, ε, γ, or µ. Useful light chain constant regions include any of the two isotypes: κ and λ.

As used herein, the term “antigenic determinant” is synonymous with “antigen” and “epitope,” and refers to a site (e.g. a contiguous stretch of amino acids or a conformational configuration made up of different regions of non-contiguous amino acids) on a polypeptide macromolecule to which an antigen binding moiety binds, forming an antigen binding moiety-antigen complex. Useful antigenic determinants can be found, for example, on the surfaces of tumor cells, on the surfaces of virus-infected cells, on the surfaces of other diseased cells, on the surface of immune cells, free in blood serum, and/or in the extracellular matrix (ECM). The proteins referred to as antigens herein (e.g. MCSP, FAP, CEA, EGFR, CD33, CD3) can be any native form the proteins from any vertebrate source, including mammals such as primates (e.g. humans) and rodents (e.g. mice and rats), unless otherwise indicated. In a particular embodiment the antigen is a human protein. Where reference is made to a specific protein herein, the term encompasses the “full-length”, unprocessed protein as well as any form of the protein that results from processing in the cell. The term also encompasses naturally occurring variants of the protein, e.g. splice variants or allelic variants. Exemplary human proteins useful as antigens include, but are not limited to: Melanoma-associated Chondroitin Sulfate Proteoglycan (MCSP), also known as Chondroitin Sulfate Proteoglycan 4 (UniProt no. Q6UVK1 (version 70), NCBI RefSeq no. NP_001888.2); Fibroblast Activation Protein (FAP), also known as Seprase (Uni Prot nos. Q12884, Q86Z29, Q99998, NCBI Accession no. NP_004451); Carcinoembroynic antigen (CEA), also known as Carcinoembryonic antigen-related cell adhesion molecule 5 (UniProt no. P06731 (version 119), NCBI RefSeq no. NP_004354.2); CD33, also known as gp67 or Siglec-3 (UniProt no. P20138, NCBI Accession nos. NP_001076087, NP_001171079); Epidermal Growth Factor Receptor (EGFR), also known as ErbB-1 or Her1 (UniProt no. P0053, NCBI Accession nos. NP_958439, NP_958440), and CD3, particularly the epsilon subunit of CD3 (see UniProt no. P07766 (version 130), NCBI RefSeq no. NP_000724.1, SEQ ID NO: 265 for the human sequence; or UniProt no. Q95LI5 (version 49), NCBI GenBank no. BAB71849.1, SEQ ID NO: 266 for the cynomolgus [Macaca fascicularis] sequence). In certain embodiments the T cell activating bispecific antigen binding molecule of the invention binds to an epitope of an activating T cell antigen or a target cell antigen that is conserved among the activating T cell antigen or target antigen from different species.

By “specific binding” is meant that the binding is selective for the antigen and can be discriminated from unwanted or non-specific interactions. The ability of an antigen binding moiety to bind to a specific antigenic determinant can be measured either through an enzyme-linked immunosorbent assay (ELISA) or other techniques familiar to one of skill in the art, e.g. surface plasmon resonance (SPR) technique (analyzed on a BIAcore instrument) (Liljeblad et al., Glyco J 17, 323-329 (2000)), and traditional binding assays (Heeley, Endocr Res 28, 217-229 (2002)). In one embodiment, the extent of binding of an antigen binding moiety to an unrelated protein is less than about 10% of the binding of the antigen binding moiety to the antigen as measured, e.g., by SPR. In certain embodiments, an antigen binding moiety that binds to the antigen, or an antigen binding molecule comprising that antigen binding moiety, has a dissociation constant (K_{D}) of ≤ 1 µM, ≤ 100 nM, ≤ 10 nM, ≤ 1 nM, ≤ 0.1 nM, ≤ 0.01 nM, or ≤ 0.001 nM (e.g. 10⁻⁸ M or less, e.g. from 10⁻⁸ M to 10⁻¹³ M, e.g., from 10⁻⁹ M to 10⁻¹³ M).

“Affinity” refers to the strength of the sum total of non-covalent interactions between a single binding site of a molecule (e.g., a receptor) and its binding partner (e.g., a ligand). Unless indicated otherwise, as used herein, “binding affinity” refers to intrinsic binding affinity which reflects a 1:1 interaction between members of a binding pair (e.g., an antigen binding moiety and an antigen, or a receptor and its ligand). The affinity of a molecule X for its partner Y can generally be represented by the dissociation constant (K_{D}), which is the ratio of dissociation and association rate constants (k_{off} and kₒₙ, respectively). Thus, equivalent affinities may comprise different rate constants, as long as the ratio of the rate constants remains the same. Affinity can be measured by well established methods known in the art, including those described herein. A particular method for measuring affinity is Surface Plasmon Resonance (SPR).

“Reduced binding”, for example reduced binding to an Fc receptor, refers to a decrease in affinity for the respective interaction, as measured for example by SPR. For clarity the term includes also reduction of the affinity to zero (or below the detection limit of the analytic method), i.e. complete abolishment of the interaction. Conversely, “increased binding” refers to an increase in binding affinity for the respective interaction.

An “activating T cell antigen” as used herein refers to an antigenic determinant expressed on the surface of a T lymphocyte, particularly a cytotoxic T lymphocyte, which is capable of inducing T cell activation upon interaction with an antigen binding molecule. Specifically, interaction of an antigen binding molecule with an activating T cell antigen may induce T cell activation by triggering the signaling cascade of the T cell receptor complex. In a particular embodiment the activating T cell antigen is CD3.

“T cell activation” as used herein refers to one or more cellular response of a T lymphocyte, particularly a cytotoxic T lymphocyte, selected from: proliferation, differentiation, cytokine secretion, cytotoxic effector molecule release, cytotoxic activity, and expression of activation markers. The T cell activating bispecific antigen binding molecules of the invention are capable of inducing T cell activation. Suitable assays to measure T cell activation are known in the art described herein.

A “target cell antigen” as used herein refers to an antigenic determinant presented on the surface of a target cell, for example a cell in a tumor such as a cancer cell or a cell of the tumor stroma.

As used herein, the terms “first” and “second” with respect to antigen binding moieties etc., are used for convenience of distinguishing when there is more than one of each type of moiety. Use of these terms is not intended to confer a specific order or orientation of the T cell activating bispecific antigen binding molecule unless explicitly so stated.

A “Fab molecule” refers to a protein consisting of the VH and CH1 domain of the heavy chain (the “Fab heavy chain”) and the VL and CL domain of the light chain (the “Fab light chain”) of an immunoglobulin.

By “fused” is meant that the components (e.g. a Fab molecule and an Fc domain subunit) are linked by peptide bonds, either directly or via one or more peptide linkers.

As used herein, the term “single-chain” refers to a molecule comprising amino acid monomers linearly linked by peptide bonds. In certain embodiments, one of the antigen binding moieties is a single-chain Fab molecule, i.e. a Fab molecule wherein the Fab light chain and the Fab heavy chain are connected by a peptide linker to form a single peptide chain. In a particular such embodiment, the C-terminus of the Fab light chain is connected to the N-terminus of the Fab heavy chain in the single-chain Fab molecule.

By a “crossover” Fab molecule (also termed “Crossfab”) is meant a Fab molecule wherein either the variable regions or the constant regions of the Fab heavy and light chain are exchanged, i.e. the crossover Fab molecule comprises a peptide chain composed of the light chain variable region and the heavy chain constant region, and a peptide chain composed of the heavy chain variable region and the light chain constant region. For clarity, in a crossover Fab molecule wherein the variable regions of the Fab light chain and the Fab heavy chain are exchanged, the peptide chain comprising the heavy chain constant region is referred to herein as the “heavy chain” of the crossover Fab molecule. Conversely, in a crossover Fab molecule wherein the constant regions of the Fab light chain and the Fab heavy chain are exchanged, the peptide chain comprising the heavy chain variable region is referred to herein as the “heavy chain” of the crossover Fab molecule.

The term “immunoglobulin molecule” refers to a protein having the structure of a naturally occurring antibody. For example, immunoglobulins of the IgG class are heterotetrameric glycoproteins of about 150,000 daltons, composed of two light chains and two heavy chains that are disulfide-bonded. From N- to C-terminus, each heavy chain has a variable region (VH), also called a variable heavy domain or a heavy chain variable domain, followed by three constant domains (CH1, CH2, and CH3), also called a heavy chain constant region. Similarly, from N- to C-terminus, each light chain has a variable region (VL), also called a variable light domain or a light chain variable domain, followed by a constant light (CL) domain, also called a light chain constant region. The heavy chain of an immunoglobulin may be assigned to one of five types, called α (IgA), δ (IgD), ε (IgE), γ (IgG), or µ (IgM), some of which may be further divided into subtypes, e.g. γ₁ (IgG₁), γ₂ (IgG₂), γ₃ (IgG₃), γ₄ (IgG₄), α₁ (IgA₁) and α₂ (IgA₂). The light chain of an immunoglobulin may be assigned to one of two types, called kappa (κ) and lambda (λ), based on the amino acid sequence of its constant domain. An immunoglobulin essentially consists of two Fab molecules and an Fc domain, linked via the immunoglobulin hinge region.

The term “antibody” herein is used in the broadest sense and encompasses various antibody structures, including but not limited to monoclonal antibodies, polyclonal antibodies, and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′ —SH, F(ab′)₂, diabodies, linear antibodies, single-chain antibody molecules (e.g. scFv), and single-domain antibodies. For a review of certain antibody fragments, see Hudson et al., Nat Med 9, 129-134 (2003). For a review of scFv fragments, see e.g. Plückthun, in The Pharmacology of Monoclonal Antibodies, vol. 113, Rosenburg and Moore eds., Springer-Verlag, New York, pp. 269-315 (1994); see also WO 93/16185; and U.S.

Pat. Nos. 5,571,894 and 5,587,458. For discussion of Fab and F(ab′)₂ fragments comprising salvage receptor binding epitope residues and having increased in vivo half-life, see U.S. Pat. No. 5,869,046. Diabodies are antibody fragments with two antigen-binding sites that may be bivalent or bispecific. See, for example, EP 404,097; WO 1993/01161; Hudson et al., Nat Med 9, 129-134 (2003); and Hollinger et al., Proc Natl Acad Sci USA 90, 6444-6448 (1993). Triabodies and tetrabodies are also described in Hudson et al., Nat Med 9, 129-134 (2003). Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, MA; see e.g. U.S.

Pat. No. 6,248,516 B1). Antibody fragments can be made by various techniques, including but not limited to proteolytic digestion of an intact antibody as well as production by recombinant host cells (e.g. E. coli or phage), as described herein.

The term “antigen binding domain” refers to the part of an antibody that comprises the area which specifically binds to and is complementary to part or all of an antigen. An antigen binding domain may be provided by, for example, one or more antibody variable domains (also called antibody variable regions). Particularly, an antigen binding domain comprises an antibody light chain variable region (VL) and an antibody heavy chain variable region (VH).

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). See, e.g., Kindt et al., Kuby Immunology, 6^{th} ed., W.H. Freeman and Co., page 91 (2007). A single VH or VL domain may be sufficient to confer antigen-binding specificity.

The term “hypervariable region” or “HVR”, as used herein, refers to each of the regions of an antibody variable domain which are hypervariable in sequence and/or form structurally defined loops (“hypervariable loops”). Generally, native four-chain antibodies comprise six HVRs; three in the VH (H1, H2, H3), and three in the VL (L1, L2, L3). HVRs generally comprise amino acid residues from the hypervariable loops and/or from the complementarity determining regions (CDRs), the latter being of highest sequence variability and/or involved in antigen recognition. With the exception of CDR1 in VH, CDRs generally comprise the amino acid residues that form the hypervariable loops. Hypervariable regions (HVRs) are also referred to as “complementarity determining regions” (CDRs), and these terms are used herein interchangeably in reference to portions of the variable region that form the antigen binding regions. This particular region has been described by Kabat et al., U.S. Dept. of Health and Human Services, Sequences of Proteins of Immunological Interest (1983) and by Chothia et al., J Mol Biol 196:901-917 (1987), where the definitions include overlapping or subsets of amino acid residues when compared against each other.

Nevertheless, application of either definition to refer to a CDR of an antibody or variants thereof is intended to be within the scope of the term as defined and used herein. The appropriate amino acid residues which encompass the CDRs as defined by each of the above cited references are set forth below in Table 1 as a comparison. The exact residue numbers which encompass a particular CDR will vary depending on the sequence and size of the CDR. Those skilled in the art can routinely determine which residues comprise a particular CDR given the variable region amino acid sequence of the antibody.

TABLE 1

CDR Definitions¹

| CDR | Kabat | Chothia | AbM₂ |
|---|---|---|---|
| V_{H} CDR1 | 31-35 | 26-32 | 26-35 |
| V_{H} CDR2 | 50-65 | 52-58 | 50-58 |
| V_{H} CDR3 | 95-102 | 95-102 | 95-102 |
| V_{L} CDR1 | 24-34 | 26-32 | 24-34 |
| V_{L} CDR2 | 50-56 | 50-52 | 50-56 |
| V_{L} CDR3 | 89-97 | 91-96 | 89-97 |

¹ Numbering of all CDR definitions in Table 1 is according to the numbering conventions set forth by Kabat et al. (see below).
² “AbM” with a lowercase “b” as used in Table 1 refers to the CDRs as defined by Oxford Molecular’s “AbM” antibody modeling software.

Kabat et al. also defined a numbering system for variable region sequences that is applicable to any antibody. One of ordinary skill in the art can unambiguously assign this system of “Kabat numbering” to any variable region sequence, without reliance on any experimental data beyond the sequence itself. As used herein, “Kabat numbering” refers to the numbering system set forth by Kabat et al., U.S. Dept. of Health and Human Services, “Sequence of Proteins of Immunological Interest” (1983). Unless otherwise specified, references to the numbering of specific amino acid residue positions in an antibody variable region are according to the Kabat numbering system.

The polypeptide sequences of the sequence listing (i.e., SEQ ID NOs 1, 3, 5, 7, 9, 11, 13, 15 etc.) are not numbered according to the Kabat numbering system. However, it is well within the ordinary skill of one in the art to convert the numbering of the sequences of the Sequence Listing to Kabat numbering.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

The “class” of an antibody or immunoglobulin refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and µ, respectively.

The term “Fc domain” or “Fc region” herein is used to define a C-terminal region of an immunoglobulin heavy chain that contains at least a portion of the constant region. The term includes native sequence Fc regions and variant Fc regions. Although the boundaries of the Fc region of an IgG heavy chain might vary slightly, the human IgG heavy chain Fc region is usually defined to extend from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991. A “subunit” of an Fc domain as used herein refers to one of the two polypeptides forming the dimeric Fc domain, i.e. a polypeptide comprising C-terminal constant regions of an immunoglobulin heavy chain, capable of stable self-association. For example, a subunit of an IgG Fc domain comprises an IgG CH2 and an IgG CH3 constant domain.

A “modification promoting the association of the first and the second subunit of the Fc domain” is a manipulation of the peptide backbone or the post-translational modifications of an Fc domain subunit that reduces or prevents the association of a polypeptide comprising the Fc domain subunit with an identical polypeptide to form a homodimer. A modification promoting association as used herein particularly includes separate modifications made to each of the two Fc domain subunits desired to associate (i.e. the first and the second subunit of the Fc domain), wherein the modifications are complementary to each other so as to promote association of the two Fc domain subunits. For example, a modification promoting association may alter the structure or charge of one or both of the Fc domain subunits so as to make their association sterically or electrostatically favorable, respectively. Thus, (hetero)dimerization occurs between a polypeptide comprising the first Fc domain subunit and a polypeptide comprising the second Fc domain subunit, which might be non-identical in the sense that further components fused to each of the subunits (e.g. antigen binding moieties) are not the same. In some embodiments the modification promoting association comprises an amino acid mutation in the Fc domain, specifically an amino acid substitution. In a particular embodiment, the modification promoting association comprises a separate amino acid mutation, specifically an amino acid substitution, in each of the two subunits of the Fc domain.

The term “effector functions” refers to those biological activities attributable to the Fc region of an antibody, which vary with the antibody isotype. Examples of antibody effector functions include: C1q binding and complement dependent cytotoxicity (CDC), Fc receptor binding, antibody-dependent cell-mediated cytotoxicity (ADCC), antibody-dependent cellular phagocytosis (ADCP), cytokine secretion, immune complex-mediated antigen uptake by antigen presenting cells, down regulation of cell surface receptors (e.g. B cell receptor), and B cell activation.

As used herein, the terms “engineer, engineered, engineering”, are considered to include any manipulation of the peptide backbone or the post-translational modifications of a naturally occurring or recombinant polypeptide or fragment thereof. Engineering includes modifications of the amino acid sequence, of the glycosylation pattern, or of the side chain group of individual amino acids, as well as combinations of these approaches.

The term “amino acid mutation” as used herein is meant to encompass amino acid substitutions, deletions, insertions, and modifications. Any combination of substitution, deletion, insertion, and modification can be made to arrive at the final construct, provided that the final construct possesses the desired characteristics, e.g., reduced binding to an Fc receptor, or increased association with another peptide. Amino acid sequence deletions and insertions include amino- and/or carboxy-terminal deletions and insertions of amino acids. Particular amino acid mutations are amino acid substitutions. For the purpose of altering e.g. the binding characteristics of an Fc region, non-conservative amino acid substitutions, i.e. replacing one amino acid with another amino acid having different structural and/or chemical properties, are particularly preferred. Amino acid substitutions include replacement by non-naturally occurring amino acids or by naturally occurring amino acid derivatives of the twenty standard amino acids (e.g. 4-hydroxyproline, 3-methylhistidine, ornithine, homoserine, 5-hydroxylysine). Amino acid mutations can be generated using genetic or chemical methods well known in the art. Genetic methods may include site-directed mutagenesis, PCR, gene synthesis and the like. It is contemplated that methods of altering the side chain group of an amino acid by methods other than genetic engineering, such as chemical modification, may also be useful. Various designations may be used herein to indicate the same amino acid mutation. For example, a substitution from proline at position 329 of the Fc domain to glycine can be indicated as 329G, G329, G₃₂₉, P329G, or Pro329Gly.

As used herein, term “polypeptide” refers to a molecule composed of monomers (amino acids) linearly linked by amide bonds (also known as peptide bonds). The term “polypeptide” refers to any chain of two or more amino acids, and does not refer to a specific length of the product. Thus, peptides, dipeptides, tripeptides, oligopeptides, “protein,” “amino acid chain,” or any other term used to refer to a chain of two or more amino acids, are included within the definition of “polypeptide,” and the term “polypeptide” may be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis. A polypeptide of the invention may be of a size of about 3 or more, 5 or more, 10 or more, 20 or more, 25 or more, 50 or more, 75 or more, 100 or more, 200 or more, 500 or more, 1,000 or more, or 2,000 or more amino acids. Polypeptides may have a defined three-dimensional structure, although they do not necessarily have such structure. Polypeptides with a defined three-dimensional structure are referred to as folded, and polypeptides which do not possess a defined three-dimensional structure, but rather can adopt a large number of different conformations, and are referred to as unfolded.

By an “isolated” polypeptide or a variant, or derivative thereof is intended a polypeptide that is not in its natural milieu. No particular level of purification is required. For example, an isolated polypeptide can be removed from its native or natural environment. Recombinantly produced polypeptides and proteins expressed in host cells are considered isolated for the purpose of the invention, as are native or recombinant polypeptides which have been separated, fractionated, or partially or substantially purified by any suitable technique.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, California, or may be compiled from the source code. The ALIGN-2 program should be compiled for use on a UNIX operating system, including digital UNIX V4.0D. All sequence comparison parameters are set by the ALIGN-2 program and do not vary. In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

$$\text{100 times the fraction X/Y}$$

where X is the number of amino acid residues scored as identical matches by the sequence alignment program ALIGN-2 in that program’s alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically statedotherwise, all % amino acid sequence identity values used herein are obtained as described in the immediately preceding paragraph using the ALIGN-2 computer program.

The term “polynucleotide” refers to an isolated nucleic acid molecule or construct, e.g. messenger RNA (mRNA), virally-derived RNA, or plasmid DNA (pDNA). A polynucleotide may comprise a conventional phosphodiester bond or a non-conventional bond (e.g. an amide bond, such as found in peptide nucleic acids (PNA). The term “nucleic acid molecule” refers to any one or more nucleic acid segments, e.g. DNA or RNA fragments, present in a polynucleotide.

By “isolated” nucleic acid molecule or polynucleotide is intended a nucleic acid molecule, DNA or RNA, which has been removed from its native environment. For example, a recombinant polynucleotide encoding a polypeptide contained in a vector is considered isolated for the purposes of the present invention. Further examples of an isolated polynucleotide include recombinant polynucleotides maintained in heterologous host cells or purified (partially or substantially) polynucleotides in solution. An isolated polynucleotide includes a polynucleotide molecule contained in cells that ordinarily contain the polynucleotide molecule, but the polynucleotide molecule is present extrachromosomally or at a chromosomal location that is different from its natural chromosomal location. Isolated RNA molecules include in vivo or in vitro RNA transcripts of the present invention, as well as positive and negative strand forms, and double-stranded forms. Isolated polynucleotides or nucleic acids according to the present invention further include such molecules produced synthetically. In addition, a polynucleotide or a nucleic acid may be or may include a regulatory element such as a promoter, ribosome binding site, or a transcription terminator. By a nucleic acid or polynucleotide having a nucleotide sequence at least, for example, 95% “identical” to a reference nucleotide sequence of the present invention, it is intended that the nucleotide sequence of the polynucleotide is identical to the reference sequence except that the polynucleotide sequence may include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These alterations of the reference sequence may occur at the 5′ or 3′ terminal positions of the reference nucleotide sequence or anywhere between those terminal positions, interspersed either individually among residues in the reference sequence or in one or more contiguous groups within the reference sequence. As a practical matter, whether any particular polynucleotide sequence is at least 80%, 85%, 90%, 95%, 96%, 97%, 98% or 99% identical to a nucleotide sequence of the present invention can be determined conventionally using known computer programs, such as the ones discussed above for polypeptides (e.g. ALIGN-2).

The term “expression cassette” refers to a polynucleotide generated recombinantly or synthetically, with a series of specified nucleic acid elements that permit transcription of a particular nucleic acid in a target cell. The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid sequence to be transcribed and a promoter. In certain embodiments, the expression cassette of the invention comprises polynucleotide sequences that encode bispecific antigen binding molecules of the invention or fragments thereof.

The term “vector” or “expression vector” is synonymous with “expression construct” and refers to a DNA molecule that is used to introduce and direct the expression of a specific gene to which it is operably associated in a target cell. The term includes the vector as a self-replicating nucleic acid structure as well as the vector incorporated into the genome of a host cell into which it has been introduced. The expression vector of the present invention comprises an expression cassette. Expression vectors allow transcription of large amounts of stable mRNA. Once the expression vector is inside the target cell, the ribonucleic acid molecule or protein that is encoded by the gene is produced by the cellular transcription and/or translation machinery. In one embodiment, the expression vector of the invention comprises an expression cassette that comprises polynucleotide sequences that encode bispecific antigen binding molecules of the invention or fragments thereof.

The terms “host cell”, “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein. A host cell is any type of cellular system that can be used to generate the bispecific antigen binding molecules of the present invention. Host cells include cultured cells, e.g. mammalian cultured cells, such as CHO cells, BHK cells, NS0 cells, SP2/0 cells, YO myeloma cells, P3X63 mouse myeloma cells, PER cells, PER.C6 cells or hybridoma cells, yeast cells, insect cells, and plant cells, to name only a few, but also cells comprised within a transgenic animal, transgenic plant or cultured plant or animal tissue.

An “activating Fc receptor” is an Fc receptor that following engagement by an Fc domain of an antibody elicits signaling events that stimulate the receptor-bearing cell to perform effector functions. Human activating Fc receptors include FcγRIIIa (CD16a), FcγRI (CD64), FcγRIIa (CD32), and FcαRI (CD89).

Antibody-dependent cell-mediated cytotoxicity (ADCC) is an immune mechanism leading to the lysis of antibody-coated target cells by immune effector cells. The target cells are cells to which antibodies or derivatives thereof comprising an Fc region specifically bind, generally via the protein part that is N-terminal to the Fc region. As used herein, the term “reduced ADCC” is defined as either a reduction in the number of target cells that are lysed in a given time, at a given concentration of antibody in the medium surrounding the target cells, by the mechanism of ADCC defined above, and/or an increase in the concentration of antibody in the medium surrounding the target cells, required to achieve the lysis of a given number of target cells in a given time, by the mechanism of ADCC. The reduction in ADCC is relative to the ADCC mediated by the same antibody produced by the same type of host cells, using the same standard production, purification, formulation and storage methods (which are known to those skilled in the art), but that has not been engineered. For example the reduction in ADCC mediated by an antibody comprising in its Fc domain an amino acid substitution that reduces ADCC, is relative to the ADCC mediated by the same antibody without this amino acid substitution in the Fc domain. Suitable assays to measure ADCC are well known in the art (see e.g. PCT publication no. WO 2006/082515 or PCT patent application no. PCT/EP2012/055393).

An “effective amount” of an agent refers to the amount that is necessary to result in a physiological change in the cell or tissue to which it is administered.

A “therapeutically effective amount” of an agent, e.g. a pharmaceutical composition, refers to an amount effective, at dosages and for periods of time necessary, to achieve the desired therapeutic or prophylactic result. A therapeutically effective amount of an agent for example eliminates, decreases, delays, minimizes or prevents adverse effects of a disease.

An “individual” or “subject” is a mammal. Mammals include, but are not limited to, domesticated animals (e.g. cows, sheep, cats, dogs, and horses), primates (e.g. humans and non-human primates such as monkeys), rabbits, and rodents (e.g. mice and rats). Particularly, the individual or subject is a human.

The term “pharmaceutical composition” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical composition, other than an active ingredient, which is nontoxic to a subject. A pharmaceutically acceptable carrier includes, but is not limited to, a buffer, excipient, stabilizer, or preservative.

As used herein, “treatment” (and grammatical variations thereof such as “treat” or “treating”) refers to clinical intervention in an attempt to alter the natural course of a disease in the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, T cell activating bispecific antigen binding molecules of the invention are used to delay development of a disease or to slow the progression of a disease.

The term “package insert” is used to refer to instructions customarily included in commercial packages of therapeutic products, that contain information about the indications, usage, dosage, administration, combination therapy, contraindications and/or warnings concerning the use of such therapeutic products.

### DETAILED DESCRIPTION OF THE EMBODIMENTS

In a first aspect the invention provides a T cell activating bispecific antigen binding molecule comprising a first and a second antigen binding moiety, one of which is a Fab molecule capable of specific binding to an activating T cell antigen and the other one of which is a Fab molecule capable of specific binding to a target cell antigen, and an Fc domain composed of a first and a second subunit capable of stable association;

wherein the first antigen binding moiety is
(a) a single chain Fab molecule wherein the Fab light chain and the Fab heavy chain are connected by a peptide linker, or
(b) a crossover Fab molecule wherein either the variable or the constant regions of the Fab light chain and the Fab heavy chain are exchanged.

### T Cell Activating Bispecific Antigen Binding Molecule Formats

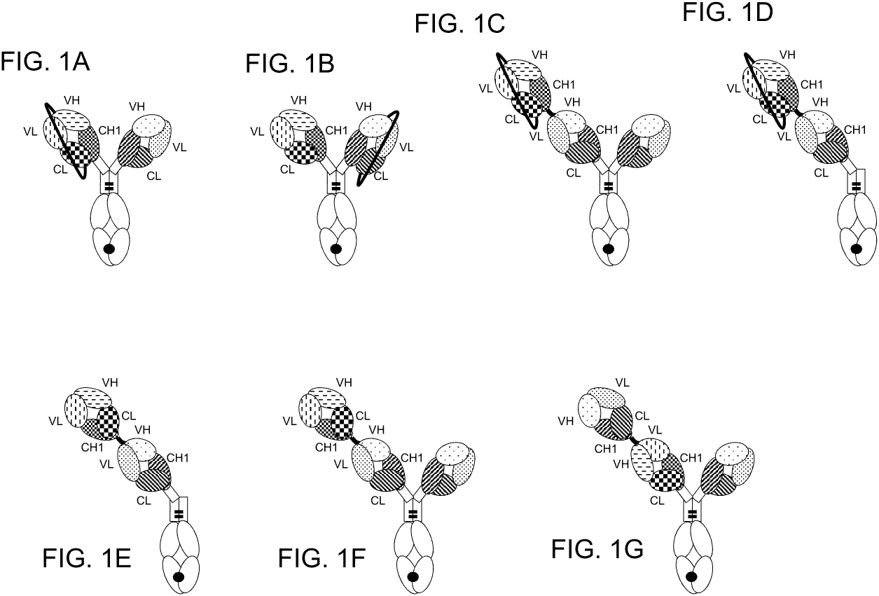

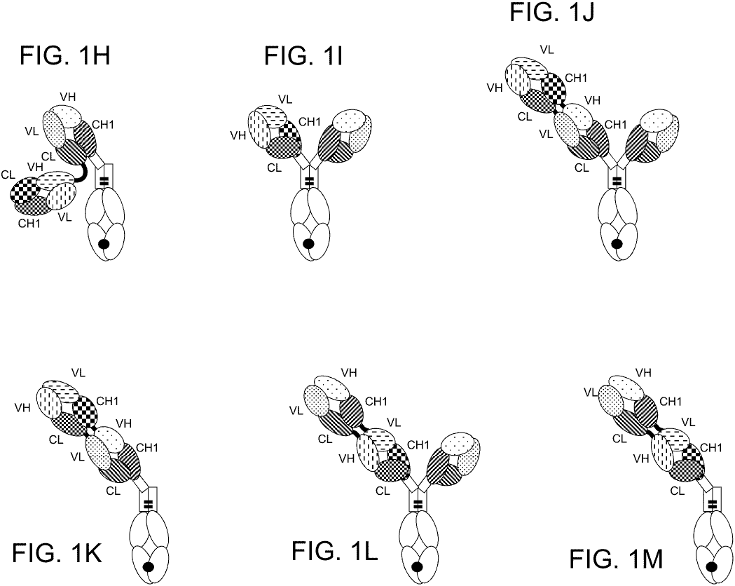

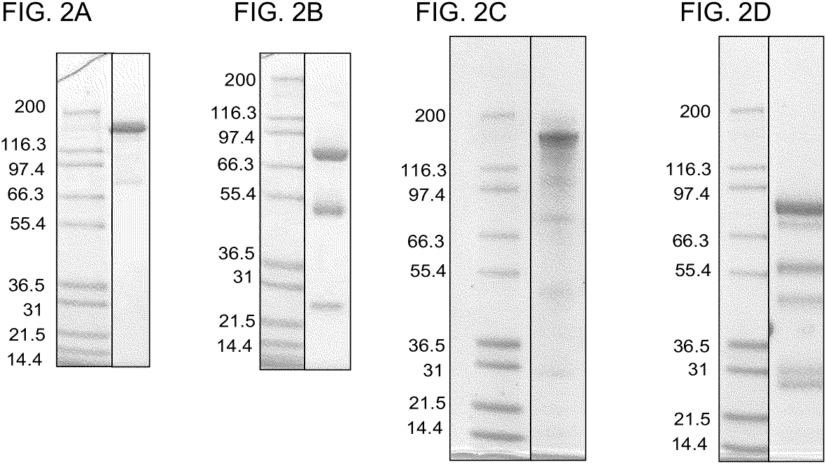

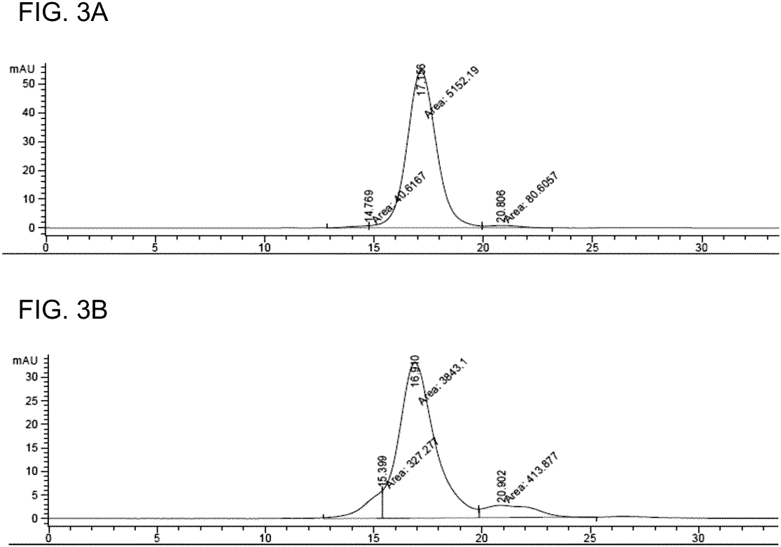

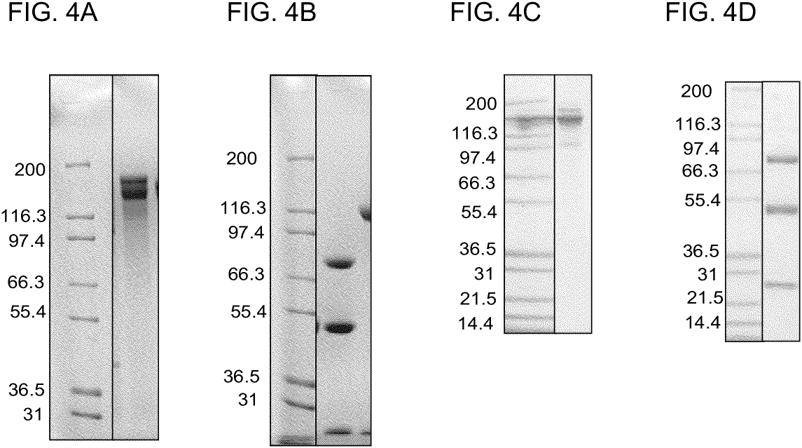

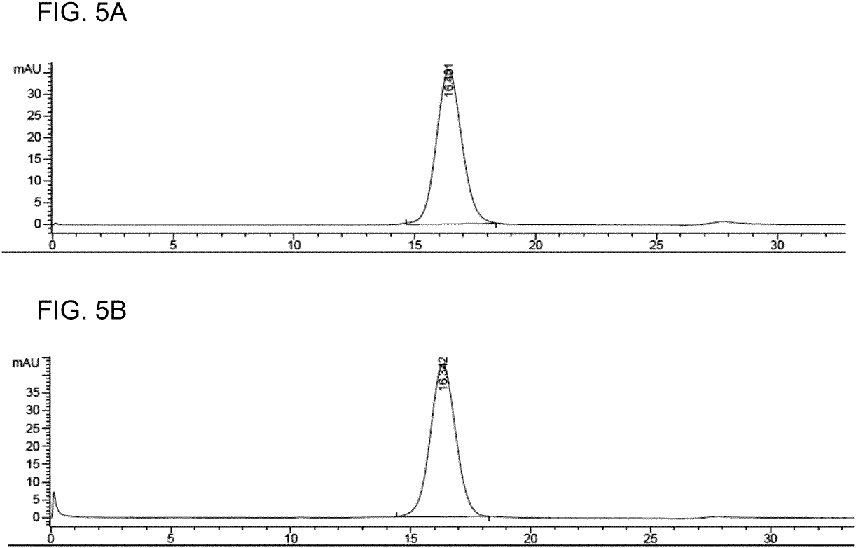

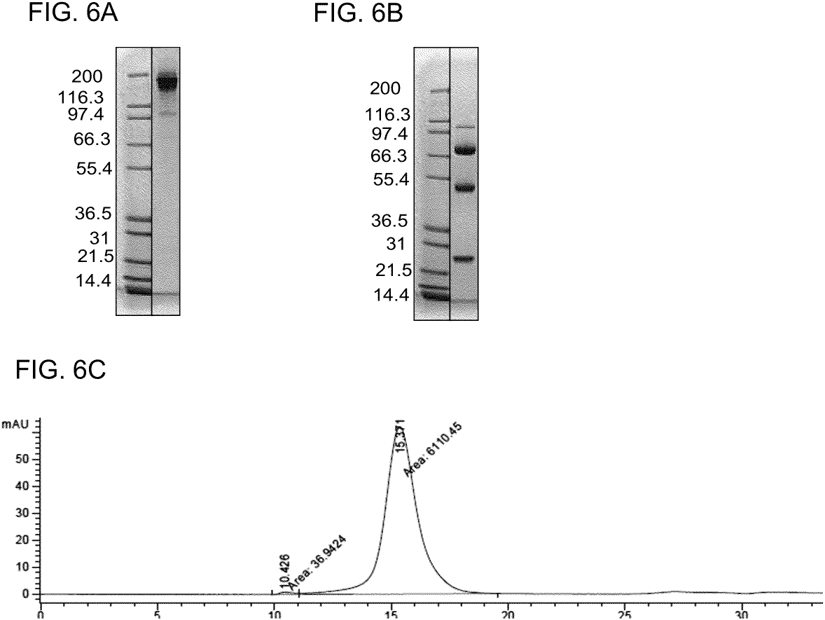

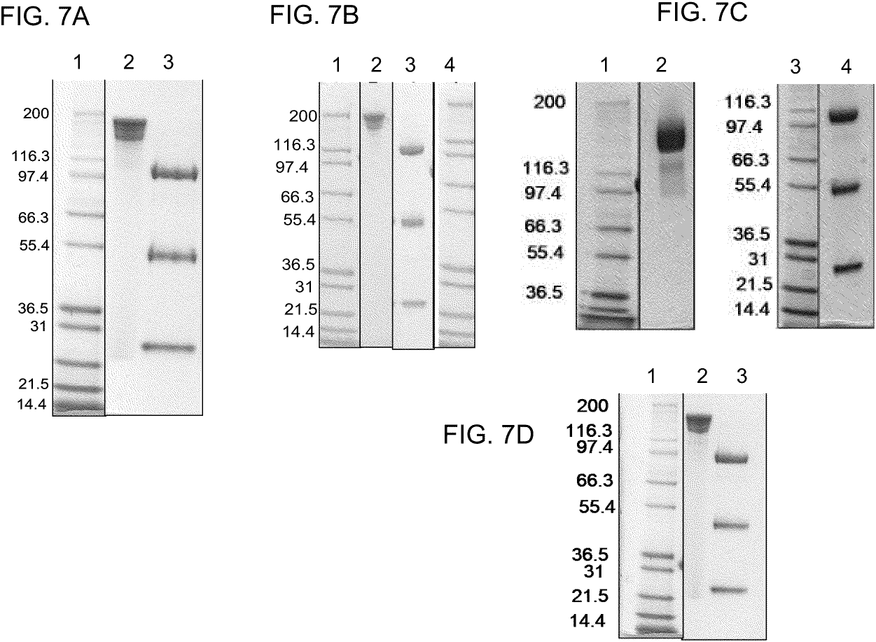

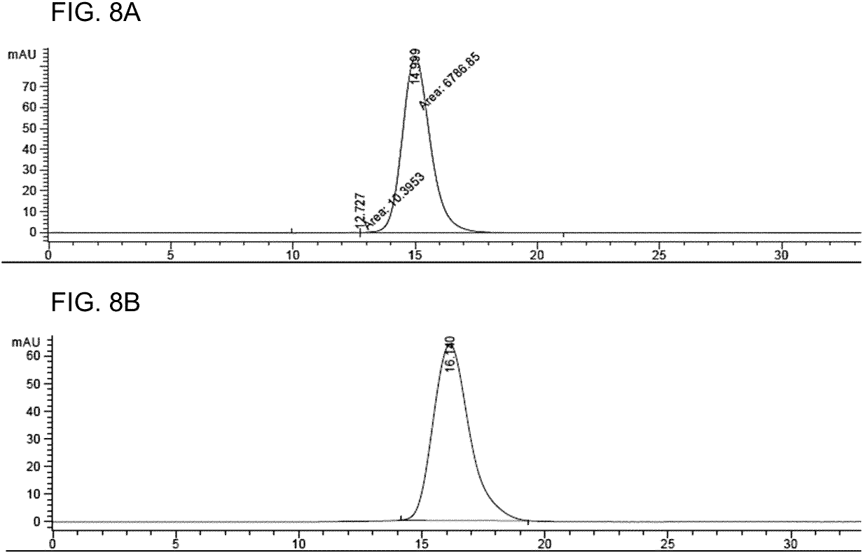

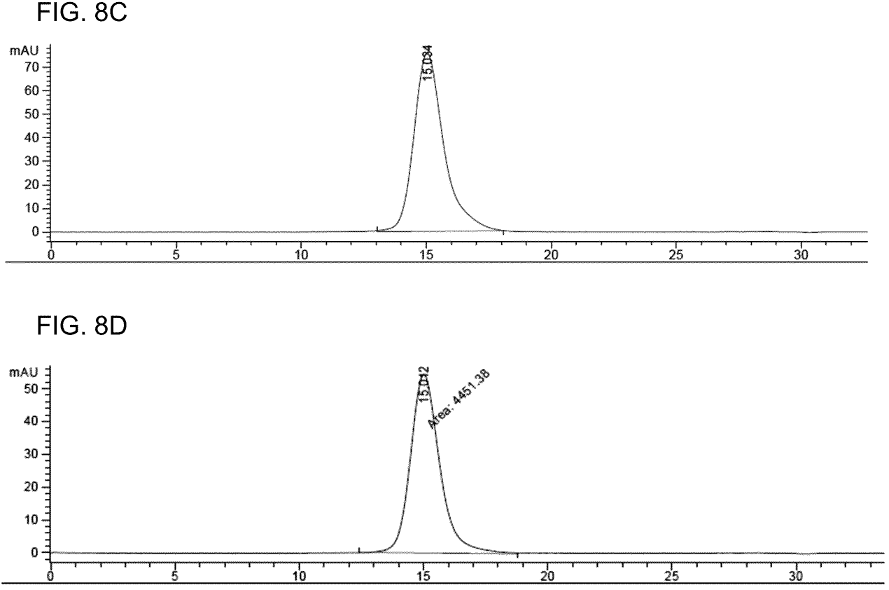

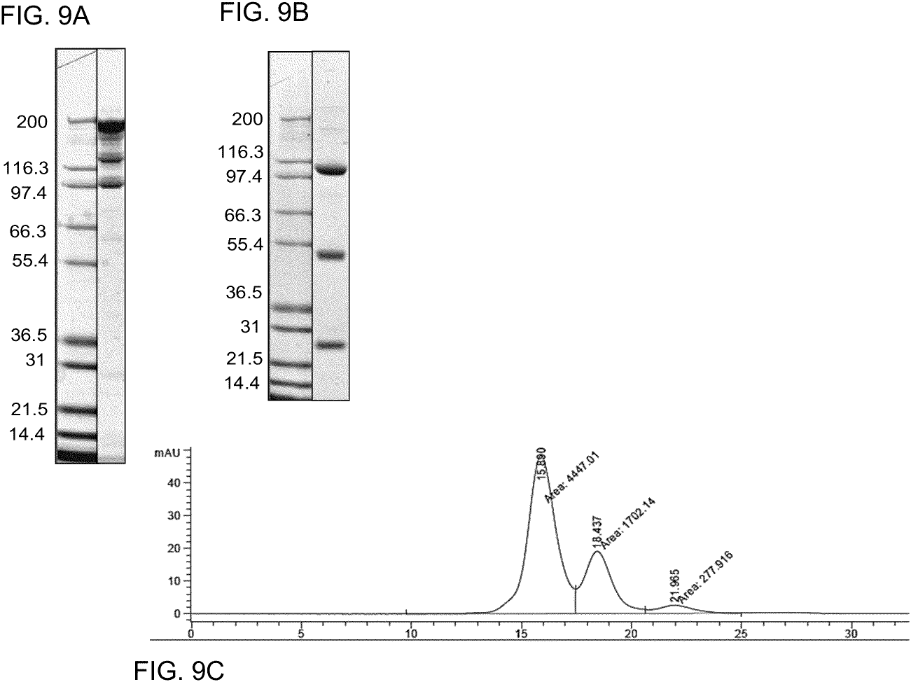

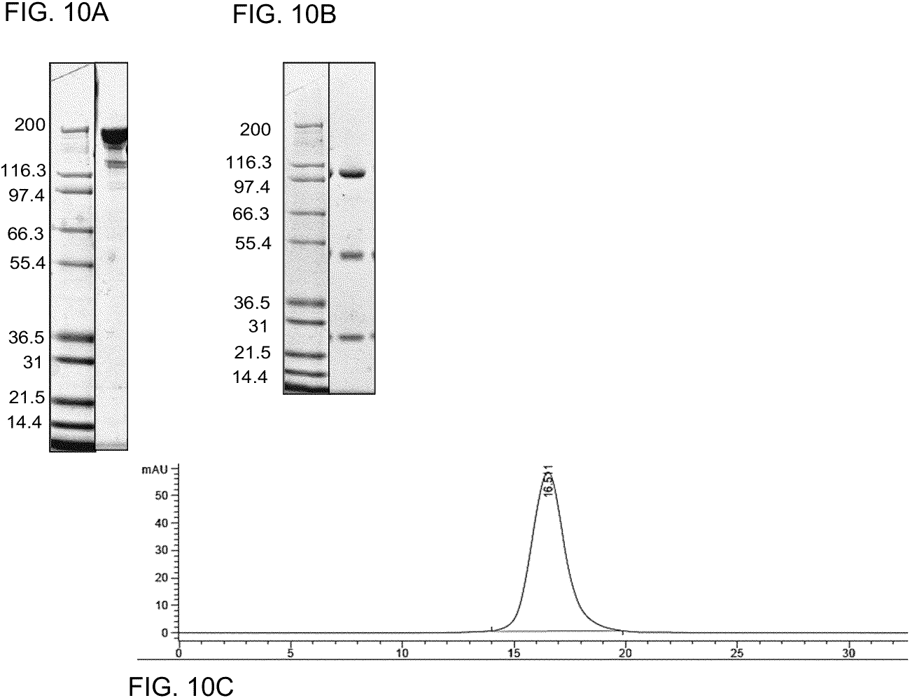

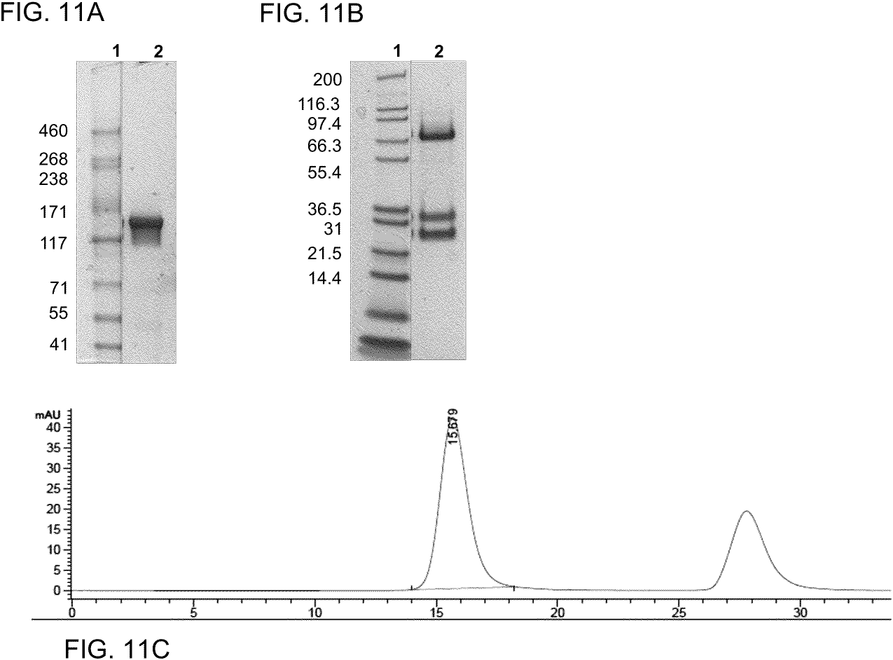

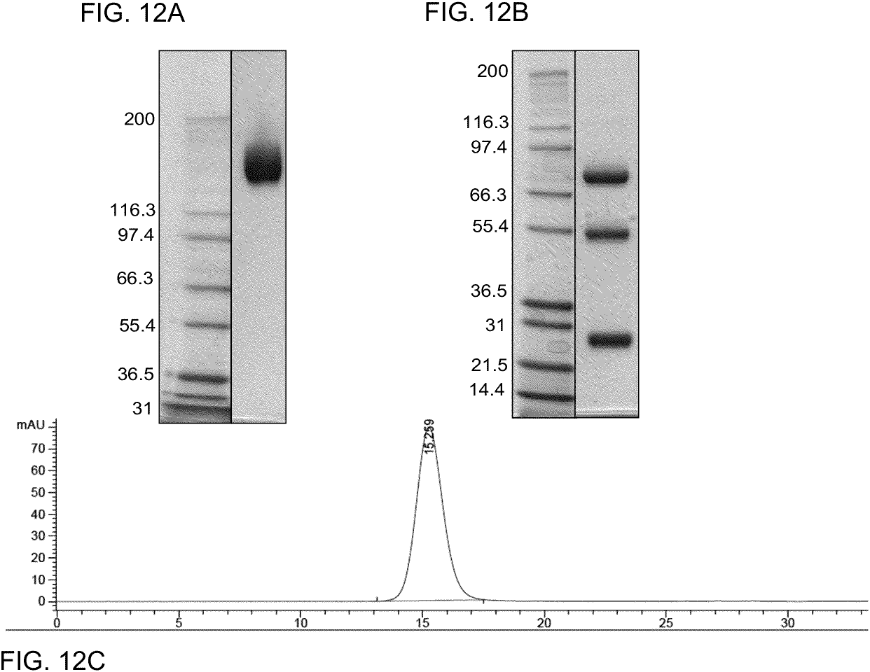

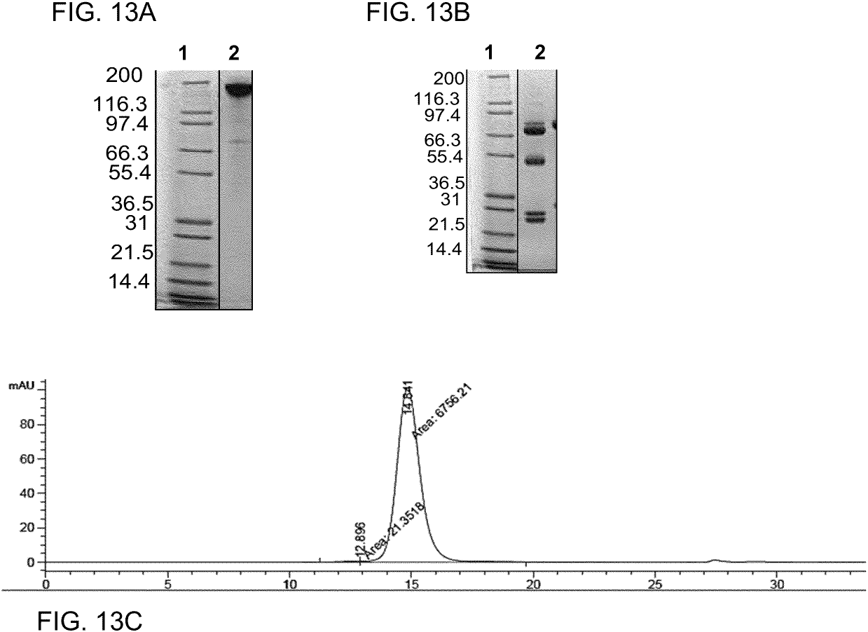

The components of the T cell activating bispecific antigen binding molecule can be fused to each other in a variety of configurations. Exemplary configurations are depicted in FIG. 1A – FIG. 1M.

In some embodiments, the second antigen binding moiety is fused at the C-terminus of the Fab heavy chain to the N-terminus of the first or the second subunit of the Fc domain.

In a particular such embodiment, the first antigen binding moiety is fused at the C-terminus of the Fab heavy chain to the N-terminus of the Fab heavy chain of the second antigen binding moiety. In a specific such embodiment, the T cell activating bispecific antigen binding molecule essentially consists of a first and a second antigen binding moiety, an Fc domain composed of a first and a second subunit, and optionally one or more peptide linkers, wherein the first antigen binding moiety is fused at the C-terminus of the Fab heavy chain to the N-terminus of the Fab heavy chain of the second antigen binding moiety, and the second antigen binding moiety is fused at the C-terminus of the Fab heavy chain to the N-terminus of the first or the second subunit of the Fc domain. In an even more specific embodiment, the first antigen binding moiety is a single chain Fab molecule. Alternatively, in a particular embodiment, the first antigen binding moiety is a crossover Fab molecule. Optionally, if the first antigen binding moiety is a crossover Fab molecule, the Fab light chain of the first antigen binding moiety and the Fab light chain of the second antigen binding moiety may additionally be fused to each other.

In an alternative such embodiment, the first antigen binding moiety is fused at the C-terminus of the Fab heavy chain to the N-terminus of the first or second subunit of the Fc domain. In a specific such embodiment, the T cell activating bispecific antigen binding molecule essentially consists of a first and a second antigen binding moiety, an Fc domain composed of a first and a second subunit, and optionally one or more peptide linkers, wherein the first and the second antigen binding moiety are each fused at the C-terminus of the Fab heavy chain to the N-terminus of one of the subunits of the Fc domain. In an even more specific embodiment, the first antigen binding moiety is a single chain Fab molecule. Alternatively, in a particular embodiment, the first antigen binding moiety is a crossover Fab molecule.

In yet another such embodiment, the second antigen binding moiety is fused at the C-terminus of the Fab light chain to the N-terminus of the Fab light chain of the first antigen binding moiety. In a specific such embodiment, the T cell activating bispecific antigen binding molecule essentially consists of a first and a second antigen binding moiety, an Fc domain composed of a first and a second subunit, and optionally one or more peptide linkers, wherein the first antigen binding moiety is fused at the N-terminus of the Fab light chain to the C-terminus of the Fab light chain of the second antigen binding moiety, and the second antigen binding moiety is fused at the C-terminus of the Fab heavy chain to the N-terminus of the first or the second subunit of the Fc domain. In an even more specific embodiment, the first antigen binding moiety is a crossover Fab molecule.

In other embodiments, the first antigen binding moiety is fused at the C-terminus of the Fab heavy chain to the N-terminus of the first or second subunit of the Fc domain.

In a particular such embodiment, the second antigen binding moiety is fused at the C-terminus of the Fab heavy chain to the N-terminus of the Fab heavy chain of the first antigen binding moiety. In a specific such embodiment, the T cell activating bispecific antigen binding molecule essentially consists of a first and a second antigen binding moiety, an Fc domain composed of a first and a second subunit, and optionally one or more peptide linkers, wherein the second antigen binding moiety is fused at the C-terminus of the Fab heavy chain to the N-terminus of the Fab heavy chain of the first antigen binding moiety, and the first antigen binding moiety is fused at the C-terminus of the Fab heavy chain to the N-terminus of the first or the second subunit of the Fc domain. In an even more specific embodiment, the first antigen binding moiety is a crossover Fab molecule. Optionally, the Fab light chain of the first antigen binding moiety and the Fab light chain of the second antigen binding moiety may additionally be fused to each other.

In particular of these embodiments, the first antigen binding moiety is capable of specific binding to an activating T cell antigen. In other embodiments, the first antigen binding moiety is capable of specific binding to a target cell antigen.

The antigen binding moieties may be fused to the Fc domain or to each other directly or through a peptide linker, comprising one or more amino acids, typically about 2-20 amino acids. Peptide linkers are known in the art and are described herein. Suitable, non-immunogenic peptide linkers include, for example, (G₄S)ₙ, (SG₄)ₙ, (G₄S)ₙ or G₄(SG₄)ₙ peptide linkers. “n” is generally a number between 1 and 10, typically between 2 and 4. A particularly suitable peptide linker for fusing the Fab light chains of the first and the second antigen binding moiety to each other is (G₄S)₂. An exemplary peptide linker suitable for connecting the Fab heavy chains of the first and the second antigen binding moiety is EPKSC(D)-(G₄S)₂ (SEQ ID NOs 150 and 151). Additionally, linkers may comprise (a portion of) an immunoglobulin hinge region. Particularly where an antigen binding moiety is fused to the N-terminus of an Fc domain subunit, it may be fused via an immunoglobulin hinge region or a portion thereof, with or without an additional peptide linker.

A T cell activating bispecific antigen binding molecule with a single antigen binding moiety capable of specific binding to a target cell antigen (for example as shown in FIG. 1A, FIG. 1B, FIG. 1D, FIG. 1E, FIG. 1H, FIG. 1I, FIG. 1K, or FIG. 1M) is useful, particularly in cases where internalization of the target cell antigen is to be expected following binding of a high affinity antigen binding moiety. In such cases, the presence of more than one antigen binding moiety specific for the target cell antigen may enhance internalization of the target cell antigen, thereby reducing its availablity.

In many other cases, however, it will be advantageous to have a T cell activating bispecific antigen binding molecule comprising two or more antigen binding moieties specific for a target cell antigen (see examples in shown in FIG. 1C, FIG. 1F, FIG. 1G, FIG. 1J, or FIG. 1L), for example to optimize targeting to the target site or to allow crosslinking of target cell antigens.

Accordingly, in certain embodiments, the T cell activating bispecific antigen binding molecule of the invention further comprises a third antigen binding moiety which is a Fab molecule capable of specific binding to a target cell antigen. In one embodiment, the third antigen binding moiety is capable of specific binding to the same target cell antigen as the first or second antigen binding moiety. In a particular embodiment, the first antigen binding moiety is capable of specific binding to an activating T cell antigen, and the second and third antigen binding moieties are capable of specific binding to a target cell antigen.

In one embodiment, the third antigen binding moiety is fused at the C-terminus of the Fab heavy chain to the N-terminus of the first or second subunit of the Fc domain. In a particular embodiment, the second and the third antigen binding moiety are each fused at the C-terminus of the Fab heavy chain to the N-terminus of one of the subunits of the Fc domain, and the first antigen binding moiety is fused at the C-terminus of the Fab heavy chain to the N-terminus of the Fab heavy chain of the second antigen binding moiety. In one such embodiment the first antigen binding moiety is a single chain Fab molecule. In a particular such embodiment the first antigen binding moiety is a crossover Fab molecule. Optionally, if the first antigen binding moiety is a crossover Fab molecule, the Fab light chain of the first antigen binding moiety and the Fab light chain of the second antigen binding moiety may additionally be fused to each other.

The second and the third antigen binding moiety may be fused to the Fc domain directly or through a peptide linker. In a particular embodiment the second and the third antigen binding moiety are each fused to the Fc domain through an immunoglobulin hinge region. In a specific embodiment, the immunoglobulin hinge region is a human IgG₁ hinge region. In one embodiment the second and the third antigen binding moiety and the Fc domain are part of an immunoglobulin molecule. In a particular embodiment the immunoglobulin molecule is an IgG class immunoglobulin. In an even more particular embodiment the immunoglobulin is an IgG₁ subclass immunoglobulin. In another embodiment the immunoglobulin is an IgG₄ subclass immunoglobulin. In a further particular embodiment the immunoglobulin is a human immunoglobulin. In other embodiments the immunoglobulin is a chimeric immunoglobulin or a humanized immunoglobulin. In one embodiment, the T cell activating bispecific antigen binding molecule essentially consists of an immunoglobulin molecule capable of specific binding to a target cell antigen, and an antigen binding moiety capable of specific binding to an activating T cell antigen wherein the antigen binding moiety is a single chain Fab molecule or a crossover Fab molecule, particularly a crossover Fab molecule, fused to the N-terminus of one of the immunoglobulin heavy chains, optionally via a peptide linker.

In an alternative embodiment, the first and the third antigen binding moiety are each fused at the C-terminus of the Fab heavy chain to the N-terminus of one of the subunits of the Fc domain, and the second antigen binding moiety is fused at the C-terminus of the Fab heavy chain to the N-terminus of the Fab heavy chain of the first antigen binding moiety. In a specific such embodiment, the T cell activating bispecific antigen binding molecule essentially consists of a first, a second and a third antigen binding moiety, an Fc domain composed of a first and a second subunit, and optionally one or more peptide linkers, wherein the second antigen binding moiety is fused at the C-terminus of the Fab heavy chain to the N-terminus of the Fab heavy chain of the first antigen binding moiety, and the first antigen binding moiety is fused at the C-terminus of the Fab heavy chain to the N-terminus of the first subunit of the Fc domain, and wherein the third antigen binding moiety is fused at the C-terminus of the Fab heavy chain to the N-terminus of the second subunit of the Fc domain. In a particular such embodiment the first antigen binding moiety is a crossover Fab molecule. Optionally, the Fab light chain of the first antigen binding moiety and the Fab light chain of the second antigen binding moiety may additionally be fused to each other.

In some of the T cell activating bispecific antigen binding molecule of the invention, the Fab light chain of the first antigen binding moiety and the Fab light chain of the second antigen binding moiety are fused to each other, optionally via a linker peptide. Depending on the configuration of the first and the second antigen binding moiety, the Fab light chain of the first antigen binding moiety may be fused at its C-terminus to the N-terminus of the Fab light chain of the second antigen binding moiety, or the Fab light chain of the second antigen binding moiety may be fused at its C-terminus to the N-terminus of the Fab light chain of the first antigen binding moiety. Fusion of the Fab light chains of the first and the second antigen binding moiety further reduces mispairing of unmatched Fab heavy and light chains, and also reduces the number of plasmids needed for expression of some of the T cell activating bispecific antigen binding molecules of the invention.

In certain embodiments the T cell activating bispecific antigen binding molecule comprises a polypeptide wherein a first Fab light chain shares a carboxy-terminal peptide bond with a peptide linker, which in turn shares a carboxy-terminal peptide bond with a first Fab heavy chain, which in turn shares a carboxy-terminal peptide bond with an Fc domain subunit (VL—CL-linker-VH-CH1-CH2-CH2(-CH4)), and a polypeptide wherein a second Fab heavy chain shares a carboxy-terminal peptide bond with an Fc domain subunit (VH—CH1—CH2—CH3(—CH4)). In some embodiments the T cell activating bispecific antigen binding molecule further comprises a second Fab light chain polypeptide (VL—CL). In certain embodiments the polypeptides are covalently linked, e.g., by a disulfide bond.

In some embodiments, the T cell activating bispecific antigen binding molecule comprises a polypeptide wherein a first Fab light chain shares a carboxy-terminal peptide bond with a peptide linker, which in turn shares a carboxy-terminal peptide bond with a first Fab heavy chain, which in turn shares a carboxy-terminal peptide bond with a second Fab heavy chain, which in turn shares a carboxy-terminal peptide bond with an Fc domain subunit (VL—CL-linker-VH-CH1-VH-CH1-CH2-CH3(-CH4)). In one of these embodiments that T cell activating bispecific antigen binding molecule further comprises a second Fab light chain polypeptide (VL—CL). The T cell activating bispecific antigen binding molecule according to these embodiments may further comprise (i) an Fc domain subunit polypeptide (CH2—CH3(—CH4)), or (ii) a polypeptide wherein a third Fab heavy chain shares a carboxy-terminal peptide bond with an Fc domain subunit (VH—CH1—CH2—CH3(—CH4)) and a third Fab light chain polypeptide (VL—CL). In certain embodiments the polypeptides are covalently linked, e.g., by a disulfide bond.

In certain embodiments the T cell activating bispecific antigen binding molecule comprises a polypeptide wherein a first Fab light chain variable region shares a carboxy-terminal peptide bond with a first Fab heavy chain constant region (i.e. a crossover Fab heavy chain, wherein the heavy chain variable region is replaced by a light chain variable region), which in turn shares a carboxy-terminal peptide bond with an Fc domain subunit (VL—CH1—CH2—CH2(—CH4)), and a polypeptide wherein a second Fab heavy chain shares a carboxy-terminal peptide bond with an Fc domain subunit (VH—CH1—CH2—CH3(—CH4)). In some embodiments the T cell activating bispecific antigen binding molecule further comprises a polypeptide wherein a Fab heavy chain variable region shares a carboxy-terminal peptide bond with a Fab light chain constant region (VH—CL) and a Fab light chain polypeptide (VL—CL). In certain embodiments the polypeptides are covalently linked, e.g., by a disulfide bond.

In alternative embodiments the T cell activating bispecific antigen binding molecule comprises a polypeptide wherein a first Fab heavy chain variable region shares a carboxy-terminal peptide bond with a first Fab light chain constant region (i.e. a crossover Fab heavy chain, wherein the heavy chain constant region is replaced by a light chain constant region), which in turn shares a carboxy-terminal peptide bond with an Fc domain subunit (VH—CL—CH2—CH2(—CH4)), and a polypeptide wherein a second Fab heavy chain shares a carboxy-terminal peptide bond with an Fc domain subunit (VH—CH1—CH2—CH3(—CH4)). In some embodiments the T cell activating bispecific antigen binding molecule further comprises a polypeptide wherein a Fab light chain variable region shares a carboxy-terminal peptide bond with a Fab heavy chain constant region (VL—CH1) and a Fab light chain polypeptide (VL—CL). In certain embodiments the polypeptides are covalently linked, e.g., by a disulfide bond.

In some embodiments, the T cell activating bispecific antigen binding molecule comprises a polypeptide wherein a first Fab light chain variable region shares a carboxy-terminal peptide bond with a first Fab heavy chain constant region (i.e. a crossover Fab heavy chain, wherein the heavy chain variable region is replaced by a light chain variable region), which in turn shares a carboxy-terminal peptide bond with a second Fab heavy chain, which in turn shares a carboxy-terminal peptide bond with an Fc domain subunit (VL—CH1—VH—CH1—CH2—CH3(—CH4)). In other embodiments, the T cell activating bispecific antigen binding molecule comprises a polypeptide wherein a first Fab heavy chain variable region shares a carboxy-terminal peptide bond with a first Fab light chain constant region (i.e. a crossover Fab heavy chain, wherein the heavy chain constant region is replaced by a light chain constant region), which in turn shares a carboxy-terminal peptide bond with a second Fab heavy chain, which in turn shares a carboxy-terminal peptide bond with an Fc domain subunit (VH—CL—VH—CH1—CH2—CH3(—CH4)). In still other embodiments, the T cell activating bispecific antigen binding molecule comprises a polypeptide wherein a second Fab heavy chain shares a carboxy-terminal peptide bond with a first Fab light chain variable region which in turn shares a carboxy-terminal peptide bond with a first Fab heavy chain constant region (i.e. a crossover Fab heavy chain, wherein the heavy chain variable region is replaced by a light chain variable region), which in turn shares a carboxy-terminal peptide bond with an Fc domain subunit (VH—CH1—VL—CH1—CH2—CH3(—CH4)). In other embodiments, the T cell activating bispecific antigen binding molecule comprises a polypeptide wherein a second Fab heavy chain shares a carboxy-terminal peptide bond with a first Fab heavy chain variable region which in turn shares a carboxy-terminal peptide bond with a first Fab light chain constant region (i.e. a crossover Fab heavy chain, wherein the heavy chain constant region is replaced by a light chain constant region), which in turn shares a carboxy-terminal peptide bond with an Fc domain subunit (VH—CH1—VH—CL—CH2—CH3(—CH4)).

In some of these embodiments the T cell activating bispecific antigen binding molecule further comprises a crossover Fab light chain polypeptide, wherein a Fab heavy chain variable region shares a carboxy-terminal peptide bond with a Fab light chain constant region (VH—CL), and a Fab light chain polypeptide (VL—CL). In others of these embodiments the T cell activating bispecific antigen binding molecule further comprises a crossover Fab light chain polypeptide, wherein a Fab light chain variable region shares a carboxy-terminal peptide bond with a Fab heavy chain constant region (VL—CH1), and a Fab light chain polypeptide (VL—CL). In still others of these embodiments the T cell activating bispecific antigen binding molecule further comprises a polypeptide wherein a Fab light chain variable region shares a carboxy-terminal peptide bond with a Fab heavy chain constant region which in turn shares a carboxy-terminal peptide bond with a Fab light chain polypeptide (VL—CH1—VL—CL), a polypeptide wherein a Fab heavy chain variable region shares a carboxy-terminal peptide bond with a Fab light chain constant region which in turn shares a carboxy-terminal peptide bond with a Fab light chain polypeptide (VH—CL—VL—CL), a polypeptide wherein a Fab light chain polypeptide shares a carboxy-terminal peptide bond with a Fab light chain variable region which in turn shares a carboxy-terminal peptide bond with a Fab heavy chain constant region (VL—CL—VL—CH1), or a polypeptide wherein a Fab light chain polypeptide shares a carboxy-terminal peptide bond with a Fab heavy chain variable region which in turn shares a carboxy-terminal peptide bond with a Fab light chain constant region (VL—CL—VH—CL).

The T cell activating bispecific antigen binding molecule according to these embodiments may further comprise (i) an Fc domain subunit polypeptide (CH2—CH3(—CH4)), or (ii) a polypeptid wherein a third Fab heavy chain shares a carboxy-terminal peptide bond with an Fc domain subunit (VH—CH1—CH2—CH3(—CH4)) and a third Fab light chain polypeptide (VL-CL). In certain embodiments the polypeptides are covalently linked, e.g., by a disulfide bond.

In one embodiment, the T cell activating bispecific antigen binding molecule comprises a polypeptide wherein a second Fab light chain shares a carboxy-terminal peptide bond with a first Fab light chain variable region which in turn shares a carboxy-terminal peptide bond with a first Fab heavy chain constant region (i.e. a crossover Fab light chain, wherein the light chain constant region is replaced by a heavy chain constant region) (VL—CL—VL—CH1), a polypeptide wherein a second Fab heavy chain shares a carboxy-terminal peptide bond with an Fc domain subunit (VH—CH1—CH2—CH3(—CH4)), and a polypeptide wherein a first Fab heavy chain variable region shares a carboxy-terminal peptide bond with a first Fab light chain constant region (VH—CL). In another embodiment, the T cell activating bispecific antigen binding molecule comprises a polypeptide wherein a second Fab light chain shares a carboxy-terminal peptide bond with a first Fab heavy chain variable region which in turn shares a carboxy-terminal peptide bond with a first Fab light chain constant region (i.e. a crossover Fab light chain, wherein the light chain variable region is replaced by a heavy chain variable region) (VL—CL—VH—CL), a polypeptide wherein a second Fab heavy chain shares a carboxy-terminal peptide bond with an Fc domain subunit (VH—CH1—CH2—CH3(—CH4)), and a polypeptide wherein a first Fab light chain variable region shares a carboxy-terminal peptide bond with a first Fab heavy chain constant region (VL—CH1). The T cell activating bispecific antigen binding molecule according to these embodiments may further comprise (i) an Fc domain subunit polypeptide (CH2—CH3(—CH4)), or (ii) a polypeptide wherein a third Fab heavy chain shares a carboxy-terminal peptide bond with an Fc domain subunit (VH—CH1—CH2—CH3(—CH4)) and a third Fab light chain polypeptide (VL—CL). In certain embodiments the polypeptides are covalently linked, e.g., by a disulfide bond.

According to any of the above embodiments, components of the T cell activating bispecific antigen binding molecule (e.g. antigen binding moiety, Fc domain) may be fused directly or through various linkers, particularly peptide linkers comprising one or more amino acids, typically about 2-20 amino acids, that are described herein or are known in the art. Suitable, non-immunogenic peptide linkers include, for example, (G₄S)ₙ, (SG₄)ₙ, (G₄S)ₙ or G₄(SG₄)ₙ peptide linkers, wherein n is generally a number between 1 and 10, typically between 2 and 4.

### Fc Domain

The Fc domain of the T cell activating bispecific antigen binding molecule consists of a pair of polypeptide chains comprising heavy chain domains of an immunoglobulin molecule. For example, the Fc domain of an immunoglobulin G (IgG) molecule is a dimer, each subunit of which comprises the CH2 and CH3 IgG heavy chain constant domains. The two subunits of the Fc domain are capable of stable association with each other. In one embodiment the T cell activating bispecific antigen binding molecule of the invention comprises not more than one Fc domain.

In one embodiment according the invention the Fc domain of the T cell activating bispecific antigen binding molecule is an IgG Fc domain. In a particular embodiment the Fc domain is an IgG₁ Fc domain. In another embodiment the Fc domain is an IgG₄ Fc domain. In a more specific embodiment, the Fc domain is an IgG₄ Fc domain comprising an amino acid substitution at position S228 (EU numbering), particularly the amino acid substitution S228P. This amino acid substitution reduces in vivo Fab arm exchange of IgG₄ antibodies (see Stubenrauch et al., Drug Metabolism and Disposition 38, 84-91 (2010)). In a further particular embodiment the Fc domain is human. An exemplary sequence of a human IgG₁ Fc region is given in SEQ ID NO: 149.

### Fc Domain Modifications Promoting Heterodimerization

T cell activating bispecific antigen binding molecules according to the invention comprise different antigen binding moieties, fused to one or the other of the two subunits of the Fc domain, thus the two subunits of the Fc domain are typically comprised in two non-identical polypeptide chains. Recombinant co-expression of these polypeptides and subsequent dimerization leads to several possible combinations of the two polypeptides. To improve the yield and purity of T cell activating bispecific antigen binding molecules in recombinant production, it will thus be advantageous to introduce in the Fc domain of the T cell activating bispecific antigen binding molecule a modification promoting the association of the desired polypeptides.

Accordingly, in particular embodiments the Fc domain of the T cell activating bispecific antigen binding molecule according to the invention comprises a modification promoting the association of the first and the second subunit of the Fc domain. The site of most extensive protein-protein interaction between the two subunits of a human IgG Fc domain is in the CH3 domain of the Fc domain. Thus, in one embodiment said modification is in the CH3 domain of the Fc domain.

In a specific embodiment said modification is a so-called “knob-into-hole” modification, comprising a “knob” modification in one of the two subunits of the Fc domain and a “hole” modification in the other one of the two subunits of the Fc domain.

The knob-into-hole technology is described e.g. in US 5,731,168; US 7,695,936; Ridgway et al., Prot Eng 9, 617-621 (1996) and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g. tyrosine or tryptophan). Compensatory cavities of identical or similar size to the protuberances are created in the interface of the second polypeptide by replacing large amino acid side chains with smaller ones (e.g. alanine or threonine).

Accordingly, in a particular embodiment, in the CH3 domain of the first subunit of the Fc domain of the T cell activating bispecific antigen binding molecule an amino acid residue is replaced with an amino acid residue having a larger side chain volume, thereby generating a protuberance within the CH3 domain of the first subunit which is positionable in a cavity within the CH3 domain of the second subunit, and in the CH3 domain of the second subunit of the Fc domain an amino acid residue is replaced with an amino acid residue having a smaller side chain volume, thereby generating a cavity within the CH3 domain of the second subunit within which the protuberance within the CH3 domain of the first subunit is positionable.

The protuberance and cavity can be made by altering the nucleic acid encoding the polypeptides, e.g. by site-specific mutagenesis, or by peptide synthesis.

In a specific embodiment, in the CH3 domain of the first subunit of the Fc domain the threonine residue at position 366 is replaced with a tryptophan residue (T366W), and in the CH3 domain of the second subunit of the Fc domain the tyrosine residue at position 407 is replaced with a valine residue (Y407V). In one embodiment, in the second subunit of the Fc domain additionally the threonine residue at position 366 is replaced with a serine residue (T366S) and the leucine residue at position 368 is replaced with an alanine residue (L368A).

In yet a further embodiment, in the first subunit of the Fc domain additionally the serine residue at position 354 is replaced with a cysteine residue (S354C), and in the second subunit of the Fc domain additionally the tyrosine residue at position 349 is replaced by a cysteine residue (Y349C). Introduction of these two cysteine residues results in formation of a disulfide bridge between the two subunits of the Fc domain, further stabilizing the dimer (Carter, J Immunol Methods 248, 7-15 (2001)).

In a particular embodiment the antigen binding moiety capable of binding to an activating T cell antigen is fused (optionally via the antigen binding moiety capable of binding to a target cell antigen) to the first subunit of the Fc domain (comprising the “knob” modification). Without wishing to be bound by theory, fusion of the antigen binding moiety capable of binding to an activating T cell antigen to the knob-containing subunit of the Fc domain will (further) minimize the generation of antigen binding molecules comprising two antigen binding moieties capable of binding to an activating T cell antigen (steric clash of two knob-containing polypeptides).

In an alternative embodiment a modification promoting association of the first and the second subunit of the Fc domain comprises a modification mediating electrostatic steering effects, e.g. as described in PCT publication WO 2009/089004. Generally, this method involves replacement of one or more amino acid residues at the interface of the two Fc domain subunits by charged amino acid residues so that homodimer formation becomes electrostatically unfavorable but heterodimerization electrostatically favorable.

### Fc Domain Modifications Reducing Fc Receptor Binding And/or Effector Function

The Fc domain confers to the T cell activating bispecific antigen binding molecule favorable pharmacokinetic properties, including a long serum half-life which contributes to good accumulation in the target tissue and a favorable tissue-blood distribution ratio. At the same time it may, however, lead to undesirable targeting of the T cell activating bispecific antigen binding molecule to cells expressing Fc receptors rather than to the preferred antigen-bearing cells. Moreover, the co-activation of Fc receptor signaling pathways may lead to cytokine release which, in combination with the T cell activating properties and the long half-life of the antigen binding molecule, results in excessive activation of cytokine receptors and severe side effects upon systemic administration. Activation of (Fc receptor-bearing) immune cells other than T cells may even reduce efficacy of the T cell activating bispecific antigen binding molecule due to the potential destruction of T cells e.g. by NK cells.

Accordingly, in particular embodiments the Fc domain of the T cell activating bispecific antigen binding molecules according to the invention exhibits reduced binding affinity to an Fc receptor and/or reduced effector function, as compared to a native IgG₁ Fc domain. In one such embodiment the Fc domain (or the T cell activating bispecific antigen binding molecule comprising said Fc domain) exhibits less than 50%, preferably less than 20%, more preferably less than 10% and most preferably less than 5% of the binding affinity to an Fc receptor, as compared to a native IgG₁ Fc domain (or a T cell activating bispecific antigen binding molecule comprising a native IgG₁ Fc domain), and/or less than 50%, preferably less than 20%, more preferably less than 10% and most preferably less than 5% of the effector function, as compared to a native IgG₁ Fc domain domain (or a T cell activating bispecific antigen binding molecule comprising a native IgG₁ Fc domain). In one embodiment, the Fc domain domain (or the T cell activating bispecific antigen binding molecule comprising said Fc domain) does not substantially bind to an Fc receptor and/or induce effector function. In a particular embodiment the Fc receptor is an Fcγ receptor. In one embodiment the Fc receptor is a human Fc receptor. In one embodiment the Fc receptor is an activating Fc receptor. In a specific embodiment the Fc receptor is an activating human Fcγ receptor, more specifically human FcγRIIIa, FcγRI or FcγRIIa, most specifically human FcγRIIIa. In one embodiment the effector function is one or more selected from the group of CDC, ADCC, ADCP, and cytokine secretion. In a particular embodiment the effector function is ADCC. In one embodiment the Fc domain domain exhibits substantially similar binding affinity to neonatal Fc receptor (FcRn), as compared to a native IgG₁ Fc domain domain. Substantially similar binding to FcRn is achieved when the Fc domain (or the T cell activating bispecific antigen binding molecule comprising said Fc domain) exhibits greater than about 70%, particularly greater than about 80%, more particularly greater than about 90% of the binding affinity of a native IgG₁ Fc domain (or the T cell activating bispecific antigen binding molecule comprising a native IgG₁ Fc domain) to FcRn.

In certain embodiments the Fc domain is engineered to have reduced binding affinity to an Fc receptor and/or reduced effector function, as compared to a non-engineered Fc domain. In particular embodiments, the Fc domain of the T cell activating bispecific antigen binding molecule comprises one or more amino acid mutation that reduces the binding affinity of the Fc domain to an Fc receptor and/or effector function. Typically, the same one or more amino acid mutation is present in each of the two subunits of the Fc domain. In one embodiment the amino acid mutation reduces the binding affinity of the Fc domain to an Fc receptor. In one embodiment the amino acid mutation reduces the binding affinity of the Fc domain to an Fc receptor by at least 2-fold, at least 5-fold, or at least 10-fold. In embodiments where there is more than one amino acid mutation that reduces the binding affinity of the Fc domain to the Fc receptor, the combination of these amino acid mutations may reduce the binding affinity of the Fc domain to an Fc receptor by at least 10-fold, at least 20-fold, or even at least 50-fold. In one embodiment the T cell activating bispecific antigen binding molecule comprising an engineered Fc domain exhibits less than 20%, particularly less than 10%, more particularly less than 5% of the binding affinity to an Fc receptor as compared to a T cell activating bispecific antigen binding molecule comprising a non-engineered Fc domain. In a particular embodiment the Fc receptor is an Fcγ receptor. In some embodiments the Fc receptor is a human Fc receptor. In some embodiments the Fc receptor is an activating Fc receptor. In a specific embodiment the Fc receptor is an activating human Fcγ receptor, more specifically human FcγRIIIa, FcγRI or FcγRIIa, most specifically human FcγRIIIa. Preferably, binding to each of these receptors is reduced. In some embodiments binding affinity to a complement component, specifically binding affinity to C1q, is also reduced. In one embodiment binding affinity to neonatal Fc receptor (FcRn) is not reduced. Substantially similar binding to FcRn, i.e. preservation of the binding affinity of the Fc domain to said receptor, is achieved when the Fc domain (or the T cell activating bispecific antigen binding molecule comprising said Fc domain) exhibits greater than about 70% of the binding affinity of a non-engineered form of the Fc domain (or the T cell activating bispecific antigen binding molecule comprising said non-engineered form of the Fc domain) to FcRn. The Fc domain, or T cell activating bispecific antigen binding molecules of the invention comprising said Fc domain, may exhibit greater than about 80% and even greater than about 90% of such affinity. In certain embodiments the Fc domain of the T cell activating bispecific antigen binding molecule is engineered to have reduced effector function, as compared to a non-engineered Fc domain. The reduced effector function can include, but is not limited to, one or more of the following: reduced complement dependent cytotoxicity (CDC), reduced antibody-dependent cell-mediated cytotoxicity (ADCC), reduced antibody-dependent cellular phagocytosis (ADCP), reduced cytokine secretion, reduced immune complex-mediated antigen uptake by antigen-presenting cells, reduced binding to NK cells, reduced binding to macrophages, reduced binding to monocytes, reduced binding to polymorphonuclear cells, reduced direct signaling inducing apoptosis, reduced crosslinking of target-bound antibodies, reduced dendritic cell maturation, or reduced T cell priming. In one embodiment the reduced effector function is one or more selected from the group of reduced CDC, reduced ADCC, reduced ADCP, and reduced cytokine secretion. In a particular embodiment the reduced effector function is reduced ADCC. In one embodiment the reduced ADCC is less than 20% of the ADCC induced by a non-engineered Fc domain (or a T cell activating bispecific antigen binding molecule comprising a non-engineered Fc domain).

In one embodiment the amino acid mutation that reduces the binding affinity of the Fc domain to an Fc receptor and/or effector function is an amino acid substitution. In one embodiment the Fc domain comprises an amino acid substitution at a position selected from the group of E233, L234, L235, N297, P331 and P329. In a more specific embodiment the Fc domain comprises an amino acid substitution at a position selected from the group of L234, L235 and P329. In some embodiments the Fc domain comprises the amino acid substitutions L234A and L235A. In one such embodiment, the Fc domain is an IgG₁ Fc domain, particularly a human IgG₁ Fc domain. In one embodiment the Fc domain comprises an amino acid substitution at position P329. In a more specific embodiment the amino acid substitution is P329A or P329G, particularly P329G. In one embodiment the Fc domain comprises an amino acid substitution at position P329 and a further amino acid substitution at a position selected from E233, L234, L235, N297 and P331. In a more specific embodiment the further amino acid substitution is E233P, L234A, L235A, L235E, N297A, N297D or P331S. In particular embodiments the Fc domain comprises amino acid substitutions at positions P329, L234 and L235. In more particular embodiments the Fc domain comprises the amino acid mutations L234A, L235A and P329G (“P329G LALA”). In one such embodiment, the Fc domain is an IgG₁ Fc domain, particularly a human IgG₁ Fc domain. The “P329G LALA” combination of amino acid substitutions almost completely abolishes Fcγ receptor binding of a human IgG₁ Fc domain, as described in PCT patent application no. PCT/EP2012/055393, incorporated herein by reference in its entirety. PCT/EP2012/055393 also describes methods of preparing such mutant Fc domains and methods for determining its properties such as Fc receptor binding or effector functions.

IgG₄ antibodies exhibit reduced binding affinity to Fc receptors and reduced effector functions as compared to IgG₁ antibodies. Hence, in some embodiments the Fc domain of the T cell activating bispecific antigen binding molecules of the invention is an IgG₄ Fc domain, particularly a human IgG₄ Fc domain. In one embodiment the IgG₄ Fc domain comprises amino acid substitutions at position S228, specifically the amino acid substitution S228P. To further reduce its binding affinity to an Fc receptor and/or its effector function, in one embodiment the IgG₄ Fc domain comprises an amino acid substitution at position L235, specifically the amino acid substitution L235E. In another embodiment, the IgG₄ Fc domain comprises an amino acid substitution at position P329, specifically the amino acid substitution P329G. In a particular embodiment, the IgG₄ Fc domain comprises amino acid substitutions at positions S228, L235 and P329, specifically amino acid substitutions S228P, L235E and P329G. Such IgG₄ Fc domain mutants and their Fcγ receptor binding properties are described in PCT patent application no. PCT/EP2012/055393, incorporated herein by reference in its entirety.

In a particular embodiment the Fc domain exhibiting reduced binding affinity to an Fc receptor and/or reduced effector function, as compared to a native IgG₁ Fc domain, is a human IgG₁ Fc domain comprising the amino acid substitutions L234A, L235A and optionally P329G, or a human IgG₄ Fc domain comprising the amino acid substitutions S228P, L235E and optionally P329G.

In certain embodiments N-glycosylation of the Fc domain has been eliminated. In one such embodiment the Fc domain comprises an amino acid mutation at position N297, particularly an amino acid substitution replacing asparagine by alanine (N297A) or aspartic acid (N297D).

In addition to the Fc domains described hereinabove and in PCT patent application no. PCT/EP2012/055393, Fc domains with reduced Fc receptor binding and/or effector function also include those with substitution of one or more of Fc domain residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Mutant Fc domains can be prepared by amino acid deletion, substitution, insertion or modification using genetic or chemical methods well known in the art. Genetic methods may include site-specific mutagenesis of the encoding DNA sequence, PCR, gene synthesis, and the like. The correct nucleotide changes can be verified for example by sequencing.

Binding to Fc receptors can be easily determined e.g. by ELISA, or by Surface Plasmon Resonance (SPR) using standard instrumentation such as a BIAcore instrument (GE Healthcare), and Fc receptors such as may be obtained by recombinant expression. A suitable such binding assay is described herein. Alternatively, binding affinity of Fc domains or cell activating bispecific antigen binding molecules comprising an Fc domain for Fc receptors may be evaluated using cell lines known to express particular Fc receptors, such as human NK cells expressing FcγIIIa receptor.

Effector function of an Fc domain, or a T cell activating bispecific antigen binding molecule comprising an Fc domain, can be measured by methods known in the art. A suitable assay for measuring ADCC is described herein. Other examples of in vitro assays to assess ADCC activity of a molecule of interest are described in U.S. Pat. No. 5,500,362; Hellstrom et al. Proc Natl Acad Sci USA 83, 7059-7063 (1986) and Hellstrom et al., Proc Natl Acad Sci USA 82, 1499-1502 (1985); U.S. Pat. No. 5,821,337; Bruggemann et al., J Exp Med 166, 1351-1361 (1987). Alternatively, non-radioactive assays methods may be employed (see, for example, ACTI™ non-radioactive cytotoxicity assay for flow cytometry (CellTechnology, Inc. Mountain View, CA); and CytoTox 96® non-radioactive cytotoxicity assay (Promega, Madison, WI)). Useful effector cells for such assays include peripheral blood mononuclear cells (PBMC) and Natural Killer (NK) cells. Alternatively, or additionally, ADCC activity of the molecule of interest may be assessed in vivo, e.g. in a animal model such as that disclosed in Clynes et al., Proc Natl Acad Sci USA 95, 652-656 (1998).

In some embodiments, binding of the Fc domain to a complement component, specifically to C1q, is reduced. Accordingly, in some embodiments wherein the Fc domain is engineered to have reduced effector function, said reduced effector function includes reduced CDC. C1q binding assays may be carried out to determine whether the T cell activating bispecific antigen binding molecule is able to bind C1q and hence has CDC activity. See e.g., C1q and C3c binding ELISA in WO 2006/029879 and WO 2005/100402. To assess complement activation, a CDC assay may be performed (see, for example, Gazzano-Santoro et al., J Immunol Methods 202, 163 (1996); Cragg et al., Blood 101, 1045-1052 (2003); and Cragg and Glennie, Blood 103, 2738-2743 (2004)).

### Antigen Binding Moieties

The antigen binding molecule of the invention is bispecific, i.e. it comprises at least two antigen binding moieties capable of specific binding to two distinct antigenic determinants. According to the invention, the antigen binding moieties are Fab molecules (i.e. antigen binding domains composed of a heavy and a light chain, each comprising a variable and a constant region). In one embodiment said Fab molecules are human. In another embodiment said Fab molecules are humanized. In yet another embodiment said Fab molecules comprise human heavy and light chain constant regions.

At least one of the antigen binding moieties is a single chain Fab molecule or a crossover Fab molecule. Such modifications prevent mispairing of heavy and light chains from different Fab molecules, thereby improving the yield and purity of the T cell activating bispecific antigen binding molecule of the invention in recombinant production. In a particular single chain Fab molecule useful for the T cell activating bispecific antigen binding molecule of the invention, the C-terminus of the Fab light chain is connected to the N-terminus of the Fab heavy chain by a peptide linker. The peptide linker allows arrangement of the Fab heavy and light chain to form a functional antigen binding moiety. Peptide linkers suitable for connecting the Fab heavy and light chain include, for example, (G₄S)₆-GG (SEQ ID NO: 152) or (SG₃)₂-(SEG₃)₄-(SG₃)-SG (SEQ ID NO: 153). In a particular crossover Fab molecule useful for the T cell activating bispecific antigen binding molecule of the invention, the constant regions of the Fab light chain and the Fab heavy chain are exchanged. In another crossover Fab molecule useful for the T cell activating bispecific antigen binding molecule of the invention, the variable regions of the Fab light chain and the Fab heavy chain are exchanged.

In a particular embodiment according to the invention, the T cell activating bispecific antigen binding molecule is capable of simultaneous binding to a target cell antigen, particularly a tumor cell antigen, and an activating T cell antigen. In one embodiment, the T cell activating bispecific antigen binding molecule is capable of crosslinking a T cell and a target cell by simultaneous binding to a target cell antigen and an activating T cell antigen. In an even more particular embodiment, such simultaneous binding results in lysis of the target cell, particularly a tumor cell. In one embodiment, such simultaneous binding results in activation of the T cell. In other embodiments, such simultaneous binding results in a cellular response of a T lymphocyte, particularly a cytotoxic T lymphocyte, selected from the group of: proliferation, differentiation, cytokine secretion, cytotoxic effector molecule release, cytotoxic activity, and expression of activation markers. In one embodiment, binding of the T cell activating bispecific antigen binding molecule to the activating T cell antigen without simultaneous binding to the target cell antigen does not result in T cell activation.

In one embodiment, the T cell activating bispecific antigen binding molecule is capable of redirecting cytotoxic activity of a T cell to a target cell. In a particular embodiment, said re-direction is independent of MHC-mediated peptide antigen presentation by the target cell and and/or specificity of the T cell.

Particularly, a T cell according to any of the embodiments of the invention is a cytotoxic T cell. In some embodiments the T cell is a CD4⁺ or a CD8⁺ T cell, particularly a CD8⁺ T cell.

### Activating T Cell Antigen Binding Moiety

The T cell activating bispecific antigen binding molecule of the invention comprises at least one antigen binding moiety capable of binding to an activating T cell antigen (also referred to herein as an “activating T cell antigen binding moiety”). In a particular embodiment, the T cell activating bispecific antigen binding molecule comprises not more than one antigen binding moiety capable of specific binding to an activating T cell antigen. In one embodiment the T cell activating bispecific antigen binding molecule provides monovalent binding to the activating T cell antigen. The activating T cell antigen binding moiety can either be a conventional Fab molecule or a modified Fab molecule, i.e. a single chain or crossover Fab molecule. In embodiments where there is more than one antigen binding moiety capable of specific binding to a target cell antigen comprised in the T cell activating bispecific antigen binding molecule, the antigen binding moiety capable of specific binding to an activating T cell antigen preferably is a modified Fab molecule.

In a particular embodiment the activating T cell antigen is CD3, particularly human CD3 (SEQ ID NO: 265) or cynomolgus CD3 (SEQ ID NO: 266), most particularly human CD3. In a particular embodiment the activating T cell antigen binding moiety is cross-reactive for (i.e. specifically binds to) human and cynomolgus CD3. In some embodiments, the activating T cell antigen is the epsilon subunit of CD3.

In one embodiment, the activating T cell antigen binding moiety can compete with monoclonal antibody H2C (described in PCT publication no. WO2008/119567) for binding an epitope of CD3. In another embodiment, the activating T cell antigen binding moiety can compete with monoclonal antibody V9 (described in Rodrigues et al., Int J Cancer Suppl 7, 45-50 (1992) and U.S. Pat. No. 6,054,297) for binding an epitope of CD3. In yet another embodiment, the activating T cell antigen binding moiety can compete with monoclonal antibody FN18 (described in Nooij et al., Eur J Immunol 19, 981-984 (1986)) for binding an epitope of CD3. In a particular embodiment, the activating T cell antigen binding moiety can compete with monoclonal antibody SP34 (described in Pessano et al., EMBO J 4, 337-340 (1985)) for binding an epitope of CD3. In one embodiment, the activating T cell antigen binding moiety binds to the same epitope of CD3 as monoclonal antibody SP34. In one embodiment, the activating T cell antigen binding moiety comprises the heavy chain CDR1 of SEQ ID NO: 163, the heavy chain CDR2 of SEQ ID NO: 165, the heavy chain CDR3 of SEQ ID NO: 167, the light chain CDR1 of SEQ ID NO: 171, the light chain CDR2 of SEQ ID NO: 173, and the light chain CDR3 of SEQ ID NO: 175. In a further embodiment, the activating T cell antigen binding moiety comprises a heavy chain variable region sequence that is at least about 80%, 85%, 90%, 95%, 96%, 97%, 98%, 99% or 100% identical to SEQ ID NO: 169 and a light chain variable region sequence that is at least about 80%, 85%, 90%, 95%, 96%, 97%, 98%, 99% or 100% identical to SEQ ID NO: 177, or variants thereof that retain functionality.

In a particular embodiment, the activating T cell antigen binding moiety comprises the heavy chain CDR1 of SEQ ID NO: 249, the heavy chain CDR2 of SEQ ID NO: 251, the heavy chain CDR3 of SEQ ID NO: 253, the light chain CDR1 of SEQ ID NO: 257, the light chain CDR2 of SEQ ID NO: 259, and the light chain CDR3 of SEQ ID NO: 261. In one embodiment, the activating T cell antigen binding moiety can compete for binding an epitope of CD3 with an antigen binding moiety comprising the heavy chain CDR1 of SEQ ID NO: 249, the heavy chain CDR2 of SEQ ID NO: 251, the heavy chain CDR3 of SEQ ID NO: 253, the light chain CDR1 of SEQ ID NO: 257, the light chain CDR2 of SEQ ID NO: 259, and the light chain CDR3 of SEQ ID NO: 261. In one embodiment, the activating T cell antigen binding moiety binds to the same epitope of CD3 as an antigen binding moiety comprising the heavy chain CDR1 of SEQ ID NO: 249, the heavy chain CDR2 of SEQ ID NO: 251, the heavy chain CDR3 of SEQ ID NO: 253, the light chain CDR1 of SEQ ID NO: 257, the light chain CDR2 of SEQ ID NO: 259, and the light chain CDR3 of SEQ ID NO: 261. In a further embodiment, the activating T cell antigen binding moiety comprises a heavy chain variable region sequence that is at least about 80%, 85%, 90%, 95%, 96%, 97%, 98%, 99% or 100% identical to SEQ ID NO: 255 and a light chain variable region sequence that is at least about 80%, 85%, 90%, 95%, 96%, 97%, 98%, 99% or 100% identical to SEQ ID NO: 263, or variants thereof that retain functionality. In one embodiment, the activating T cell antigen binding moiety can compete for binding an epitope of CD3 with an antigen binding moiety comprising the heavy chain variable region sequence of SEQ ID NO: 255 and the light chain variable region sequence of SEQ ID NO: 263. In one embodiment, the activating T cell antigen binding moiety binds to the same epitope of CD3 as an antigen binding moiety comprising the heavy chain variable region sequence of SEQ ID NO: 255 and the light chain variable region sequence of SEQ ID NO: 263. In another embodiment, the activating T cell antigen binding moiety comprises a humanized version of the heavy chain variable region sequence of SEQ ID NO: 255 and a humanized version of the light chain variable region sequence of SEQ ID NO: 263. In one embodiment, the activating T cell antigen binding moiety comprises the heavy chain CDR1 of SEQ ID NO: 249, the heavy chain CDR2 of SEQ ID NO: 251, the heavy chain CDR3 of SEQ ID NO: 253, the light chain CDR1 of SEQ ID NO: 257, the light chain CDR2 of SEQ ID NO: 259, the light chain CDR3 of SEQ ID NO: 261, and human heavy and light chain variable region framework sequences.

### Target Cell Antigen Binding Moiety

The T cell activating bispecific antigen binding molecule of the invention comprises at least one antigen binding moiety capable of binding to a target cell antigen (also referred to herein as an “target cell antigen binding moiety”). In certain embodiments, the T cell activating bispecific antigen binding molecule comprises two antigen binding moieties capable of binding to a target cell antigen. In a particular such embodiment, each of these antigen binding moieties specifically binds to the same antigenic determinant. In one embodiment, the T cell activating bispecific antigen binding molecule comprises an immunoglobulin molecule capable of specific binding to a target cell antigen. In one embodiment the T cell activating bispecific antigen binding molecule comprises not more than two antigen binding moieties capable of binding to a target cell antigen.

The target cell antigen binding moiety is generally a Fab molecule that binds to a specific antigenic determinant and is able to direct the T cell activating bispecific antigen binding molecule to a target site, for example to a specific type of tumor cell that bears the antigenic determinant.

In certain embodiments the target cell antigen binding moiety is directed to an antigen associated with a pathological condition, such as an antigen presented on a tumor cell or on a virus-infected cell. Suitable antigens are cell surface antigens, for example, but not limited to, cell surface receptors. In particular embodiments the antigen is a human antigen. In a specific embodiment the target cell antigen is selected from the group of Fibroblast Activation Protein (FAP), Melanoma-associated Chondroitin Sulfate Proteoglycan (MCSP), Epidermal Growth Factor Receptor (EGFR), Carcinoembryonic Antigen (CEA),CD19, CD20 and CD33.

In particular embodiments the T cell activating bispecific antigen binding molecule comprises at least one antigen binding moiety that is specific for Melanoma-associated Chondroitin Sulfate Proteoglycan (MCSP). In one embodiment the T cell activating bispecific antigen binding molecule comprises at least one, typically two or more antigen binding moieties that can compete with monoclonal antibody LC007 (see SEQ ID NOs 75 and 83, and European patent application no. EP 11178393.2, incorporated herein by reference in its entirety) for binding to an epitope of MCSP. In one embodiment, the antigen binding moiety that is specific for MCSP comprises the heavy chain CDR1 of SEQ ID NO: 69, the heavy chain CDR2 of SEQ ID NO: 71, the heavy chain CDR3 of SEQ ID NO: 73, the light chain CDR1 of SEQ ID NO: 77, the light chain CDR2 of SEQ ID NO: 79, and the light chain CDR3 of SEQ ID NO: 81. In a further embodiment, the antigen binding moiety that is specific for MCSP comprises a heavy chain variable region sequence that is at least about 80%, 85%, 90%, 95%, 96%, 97%, 98%, 99% or 100% identical to SEQ ID NO: 75 and a light chain variable region sequence that is at least about 80%, 85%, 90%, 95%, 96%, 97%, 98%, 99% or 100% identical to SEQ ID NO: 83, or variants thereof that retain functionality. In particular embodiments the T cell activating bispecific antigen binding molecule comprises at least one, typically two or more antigen binding moieties that can compete with monoclonal antibody M4-3 ML2 (see SEQ ID NOs 239 and 247, and European patent application no. EP 11178393.2, incorporated herein by reference in its entirety) for binding to an epitope of MCSP. In one embodiment, the antigen binding moiety that is specific for MCSP binds to the same epitope of MCSP as monoclonal antibody M4-3 ML2. In one embodiment, the antigen binding moiety that is specific for MCSP comprises the heavy chain CDR1 of SEQ ID NO: 233, the heavy chain CDR2 of SEQ ID NO: 235, the heavy chain CDR3 of SEQ ID NO: 237, the light chain CDR1 of SEQ ID NO: 241, the light chain CDR2 of SEQ ID NO: 243, and the light chain CDR3 of SEQ ID NO: 245. In a further embodiment, the antigen binding moiety that is specific for MCSP comprises a heavy chain variable region sequence that is at least about 80%, 85%, 90%, 95%, 96%, 97%, 98%, 99% or 100%, particularly about 98%, 99% or 100%, identical to SEQ ID NO: 239 and a light chain variable region sequence that is at least about 80%, 85%, 90%, 95%, 96%, 97%, 98%, 99% or 100%, particularly about 98%, 99% or 100%, identical to SEQ ID NO: 247, or variants thereof that retain functionality. In one embodiment, the antigen binding moiety that is specific for MCSP comprises the heavy and light chain variable region sequences of an affinity matured version of monoclonal antibody M4-3 ML2. In one embodiment, the antigen binding moiety that is specific for MCSP comprises the heavy chain variable region sequence of SEQ ID NO: 239 with one, two, three, four, five, six or seven, particularly two, three, four or five, amino acid substitutions; and the light chain variable region sequence of SEQ ID NO: 247 with one, two, three, four, five, six or seven, particularly two, three, four or five, amino acid substitutions. Any amino acid residue within the variable region sequences may be substituted by a different amino acid, including amino acid residues within the CDR regions, provided that binding to MCSP, particularly human MCSP, is preserved. Preferred variants are those having a binding affinity for MCSP at least equal (or stronger) to the binding affinity of the antigen binding moiety comprising the unsubstituted variable region sequences.

In one embodiment the T cell activating bispecific antigen binding molecule comprises the polypeptide sequence of SEQ ID NO: 1, the polypeptide sequence of SEQ ID NO: 3 and the polypeptide sequence of SEQ ID NO: 5, or variants thereof that retain functionality. In a further embodiment the T cell activating bispecific antigen binding molecule comprises the polypeptide sequence of SEQ ID NO: 7, the polypeptide sequence of SEQ ID NO: 9 and the polypeptide sequence of SEQ ID NO: 11, or variants thereof that retain functionality. In yet another embodiment the T cell activating bispecific antigen binding molecule comprises the polypeptide sequence of SEQ ID NO: 13, the polypeptide sequence of SEQ ID NO: 15 and the polypeptide sequence of SEQ ID NO: 5, or variants thereof that retain functionality. In yet another embodiment the T cell activating bispecific antigen binding molecule comprises the polypeptide sequence of SEQ ID NO: 17, the polypeptide sequence of SEQ ID NO: 19 and the polypeptide sequence of SEQ ID NO: 5, or variants thereof that retain functionality. In another embodiment the T cell activating bispecific antigen binding molecule comprises the polypeptide sequence of SEQ ID NO: 21, the polypeptide sequence of SEQ ID NO: 23 and the polypeptide sequence of SEQ ID NO: 5, or variants thereof that retain functionality. In still another embodiment the T cell activating bispecific antigen binding molecule comprises the polypeptide sequence of SEQ ID NO: 25, the polypeptide sequence of SEQ ID NO: 27 and the polypeptide sequence of SEQ ID NO: 5, or variants thereof that retain functionality. In another embodiment the T cell activating bispecific antigen binding molecule comprises the polypeptide sequence of SEQ ID NO: 29, the polypeptide sequence of SEQ ID NO: 31, the polypeptide sequence of SEQ ID NO: 33, and the polypeptide sequence of SEQ ID NO: 5, or variants thereof that retain functionality. In another embodiment the T cell activating bispecific antigen binding molecule comprises the polypeptide sequence of SEQ ID NO: 29, the polypeptide sequence of SEQ ID NO: 3, the polypeptide sequence of SEQ ID NO: 33, and the polypeptide sequence of SEQ ID NO: 5, or variants thereof that retain functionality. In another embodiment the T cell activating bispecific antigen binding molecule comprises the polypeptide sequence of SEQ ID NO: 35, the polypeptide sequence of SEQ ID NO: 3, the polypeptide sequence of SEQ ID NO: 37, and the polypeptide sequence of SEQ ID NO: 5, or variants thereof that retain functionality. In another embodiment the T cell activating bispecific antigen binding molecule comprises the polypeptide sequence of SEQ ID NO: 39, the polypeptide sequence of SEQ ID NO: 3, the polypeptide sequence of SEQ ID NO: 41, and the polypeptide sequence of SEQ ID NO: 5, or variants thereof that retain functionality. In yet another embodiment the T cell activating bispecific antigen binding molecule comprises the polypeptide sequence of SEQ ID NO: 29, the polypeptide sequence of SEQ ID NO: 3, the polypeptide sequence of SEQ ID NO: 5 and the polypeptide sequence of SEQ ID NO: 179, or variants thereof that retain functionality. In one embodiment the T cell activating bispecific antigen binding molecule comprises the polypeptide sequence of SEQ ID NO: 5, the polypeptide sequence of SEQ ID NO: 29, the polypeptide sequence of SEQ ID NO: 33 and the polypeptide sequence of SEQ ID NO: 181, or variants thereof that retain functionality. In one embodiment the T cell activating bispecific antigen binding molecule comprises the polypeptide sequence of SEQ ID NO: 5, the polypeptide sequence of SEQ ID NO: 23, the polypeptide sequence of SEQ ID NO: 183 and the polypeptide sequence of SEQ ID NO: 185, or variants thereof that retain functionality. In one embodiment the T cell activating bispecific antigen binding molecule comprises the polypeptide sequence of SEQ ID NO: 5, the polypeptide sequence of SEQ ID NO: 23, the polypeptide sequence of SEQ ID NO: 183 and the polypeptide sequence of SEQ ID NO: 187, or variants thereof that retain functionality. In one embodiment the T cell activating bispecific antigen binding molecule comprises the polypeptide sequence of SEQ ID NO: 33, the polypeptide sequence of SEQ ID NO: 189, the polypeptide sequence of SEQ ID NO: 191 and the polypeptide sequence of SEQ ID NO: 193, or variants thereof that retain functionality. In one embodiment the T cell activating bispecific antigen binding molecule comprises the polypeptide sequence of SEQ ID NO: 183, the polypeptide sequence of SEQ ID NO: 189, the polypeptide sequence of SEQ ID NO: 193 and the polypeptide sequence of SEQ ID NO: 195, or variants thereof that retain functionality. In one embodiment the T cell activating bispecific antigen binding molecule comprises the polypeptide sequence of SEQ ID NO: 189, the polypeptide sequence of SEQ ID NO: 193, the polypeptide sequence of SEQ ID NO: 199 and the polypeptide sequence of SEQ ID NO: 201, or variants thereof that retain functionality. In one embodiment the T cell activating bispecific antigen binding molecule comprises the polypeptide sequence of SEQ ID NO: 5, the polypeptide sequence of SEQ ID NO: 23, the polypeptide sequence of SEQ ID NO: 215 and the polypeptide sequence of SEQ ID NO: 217, or variants thereof that retain functionality. In one embodiment the T cell activating bispecific antigen binding molecule comprises the polypeptide sequence of SEQ ID NO: 5, the polypeptide sequence of SEQ ID NO: 23, the polypeptide sequence of SEQ ID NO: 215 and the polypeptide sequence of SEQ ID NO: 219, or variants thereof that retain functionality.

In a specific embodiment the T cell activating bispecific antigen binding molecule comprises a polypeptide sequence encoded by a polynucleotide sequence that is at least about 80%, 85%, 90%, 95%, 96%, 97%, 98%, 99% or 100% identical to a sequence selected from the group of SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 234, SEQ ID NO: 236, SEQ ID NO: 238, SEQ ID NO: 240, SEQ ID NO: 242, SEQ ID NO: 244, SEQ ID NO: 246, SEQ ID NO: 248, SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 180, SEQ ID NO: 182, SEQ ID NO: 184, SEQ ID NO: 186, SEQ ID NO: 188, SEQ ID NO: 190, SEQ ID NO: 192, SEQ ID NO: 194, SEQ ID NO: 196, SEQ ID NO: 200, SEQ ID NO: 202, SEQ ID NO: 216, SEQ ID NO: 218 and SEQ ID NO: 220. In one embodiment the T cell activating bispecific antigen binding molecule comprises at least one antigen binding moiety that is specific for Epidermal Growth Factor Receptor (EGFR). In another embodiment the T cell activating bispecific antigen binding molecule comprises at least one, typically two or more antigen binding moieties that can compete with monoclonal antibody GA201 for binding to an epitope of EGFR. See PCT publication WO 2006/082515, incorporated herein by reference in its entirety. In one embodiment, the antigen binding moiety that is specific for EGFR comprises the heavy chain CDR1 of SEQ ID NO: 85, the heavy chain CDR2 of SEQ ID NO: 87, the heavy chain CDR3 of SEQ ID NO: 89, the light chain CDR1 of SEQ ID NO: 93, the light chain CDR2 of SEQ ID NO: 95, and the light chain CDR3 of SEQ ID NO: 97. In a further embodiment, the antigen binding moiety that is specific for EGFR comprises a heavy chain variable region sequence that is at least about 80%, 85%, 90%, 95%, 96%, 97%, 98%, 99% or 100% identical to SEQ ID NO: 91 and a light chain variable region sequence that is at least about 80%, 85%, 90%, 95%, 96%, 97%, 98%, 99% or 100% identical to SEQ ID NO: 99, or variants thereof that retain functionality.

In yet another embodiment the T cell activating bispecific antigen binding molecule comprises the polypeptide sequence of SEQ ID NO: 43, the polypeptide sequence of SEQ ID NO: 45 and the polypeptide sequence of SEQ ID NO: 47, or variants thereof that retain functionality. In a further embodiment the T cell activating bispecific antigen binding molecule comprises the polypeptide sequence of SEQ ID NO: 49, the polypeptide sequence of SEQ ID NO: 51 and the polypeptide sequence of SEQ ID NO: 11, or variants thereof that retain functionality. In yet another embodiment the T cell activating bispecific antigen binding molecule comprises the polypeptide sequence of SEQ ID NO: 53, the polypeptide sequence of SEQ ID NO: 45 and the polypeptide sequence of SEQ ID NO: 47, or variants thereof that retain functionality.

In a specific embodiment the T cell activating bispecific antigen binding molecule comprises a polypeptide sequence encoded by a polynucleotide sequence that is at least about 80%, 85%, 90%, 95%, 96%, 97%, 98%, 99% or 100% identical to a sequence selected from the group of SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 44, SEQ ID NO: 46, SEQ ID NO: 48, SEQ ID NO: 50, SEQ ID NO: 52, SEQ ID NO: 54 and SEQ ID NO: 12.

In one embodiment the T cell activating bispecific antigen binding molecule comprises at least one antigen binding moiety that is specific for Fibroblast Activation Protein (FAP). In another embodiment the T cell activating bispecific antigen binding molecule comprises at least one, typically two or more antigen binding moieties that can compete with monoclonal antibody 3F2 for binding to an epitope of FAP. See PCT publication WO 2012/020006, incorporated herein by reference in its entirety. In one embodiment, the antigen binding moiety that is specific for FAP comprises the heavy chain CDR1 of SEQ ID NO: 101, the heavy chain CDR2 of SEQ ID NO: 103, the heavy chain CDR3 of SEQ ID NO: 105, the light chain CDR1 of SEQ ID NO: 109, the light chain CDR2 of SEQ ID NO: 111, and the light chain CDR3 of SEQ ID NO: 113. In a further embodiment, the antigen binding moiety that is specific for FAP comprises a heavy chain variable region sequence that is at least about 80%, 85%, 90%, 95%, 96%, 97%, 98%, 99% or 100% identical to SEQ ID NO: 107 and a light chain variable region sequence that is at least about 80%, 85%, 90%, 95%, 96%, 97%, 98%, 99% or 100% identical to SEQ ID NO: 115, or variants thereof that retain functionality.

In yet another embodiment the T cell activating bispecific antigen binding molecule comprises the polypeptide sequence of SEQ ID NO: 55, the polypeptide sequence of SEQ ID NO: 51 and the polypeptide sequence of SEQ ID NO: 11, or variants thereof that retain functionality. In a further embodiment the T cell activating bispecific antigen binding molecule comprises the polypeptide sequence of SEQ ID NO: 57, the polypeptide sequence of SEQ ID NO: 59 and the polypeptide sequence of SEQ ID NO: 61, or variants thereof that retain functionality.

In a specific embodiment the T cell activating bispecific antigen binding molecule comprises a polypeptide sequence encoded by a polynucleotide sequence that is at least about 80%, 85%, 90%, 95%, 96%, 97%, 98%, 99% or 100% identical to a sequence selected from the group of SEQ ID NO: 102, SEQ ID NO: 104, SEQ ID NO: 106, SEQ ID NO: 108, SEQ ID NO: 110, SEQ ID NO: 112, SEQ ID NO: 114, SEQ ID NO: 116, SEQ ID NO: 56, SEQ ID NO: 58, SEQ ID NO: 60, SEQ ID NO: 62, SEQ ID NO: 52 and SEQ ID NO: 12.

In particular embodiments the T cell activating bispecific antigen binding molecule comprises at least one antigen binding moiety that is specific for Carcinoembryonic Antigen (CEA). In one embodiment the T cell activating bispecific antigen binding molecule comprises at least one, typically two or more antigen binding moieties that can compete with monoclonal antibody BW431/26 (described in European patent no. EP 160 897, and Bosslet et al., Int J Cancer 36, 75-84 (1985)) for binding to an epitope of CEA. In one embodiment the T cell activating bispecific antigen binding molecule comprises at least one, typically two or more antigen binding moieties that can compete with monoclonal antibody CH1A1A (see SEQ ID NOs 123 and 131) for binding to an epitope of CEA. See PCT patent publication number WO 2011/023787, incorporated herein by reference in its entirety. In one embodiment, the antigen binding moiety that is specific for CEA binds to the same epitope of CEA as monoclonal antibody CH1A1A. In one embodiment, the antigen binding moiety that is specific for CEA comprises the heavy chain CDR1 of SEQ ID NO: 117, the heavy chain CDR2 of SEQ ID NO: 119, the heavy chain CDR3 of SEQ ID NO: 121, the light chain CDR1 of SEQ ID NO: 125, the light chain CDR2 of SEQ ID NO: 127, and the light chain CDR3 of SEQ ID NO: 129. In a further embodiment, the antigen binding moiety that is specific for CEA comprises a heavy chain variable region sequence that is at least about 80%, 85%, 90%, 95%, 96%, 97%, 98%, 99% or 100%, particularly about 98%, 99% or 100%, identical to SEQ ID NO: 123 and a light chain variable region sequence that is at least about 80%, 85%, 90%, 95%, 96%, 97%, 98%, 99% or 100%, particularly about 98%, 99% or 100%, identical to SEQ ID NO: 131, or variants thereof that retain functionality. In one embodiment, the antigen binding moiety that is specific for CEA comprises the heavy and light chain variable region sequences of an affinity matured version of monoclonal antibody CH1A1A. In one embodiment, the antigen binding moiety that is specific for CEA comprises the heavy chain variable region sequence of SEQ ID NO: 123 with one, two, three, four, five, six or seven, particularly two, three, four or five, amino acid substitutions; and the light chain variable region sequence of SEQ ID NO: 131 with one, two, three, four, five, six or seven, particularly two, three, four or five, amino acid substitutions. Any amino acid residue within the variable region sequences may be substituted by a different amino acid, including amino acid residues within the CDR regions, provided that binding to CEA, particularly human CEA, is preserved. Preferred variants are those having a binding affinity for CEA at least equal (or stronger) to the binding affinity of the antigen binding moiety comprising the unsubstituted variable region sequences.

In one embodiment the T cell activating bispecific antigen binding molecule comprises the polypeptide sequence of SEQ ID NO: 63, the polypeptide sequence of SEQ ID NO: 65, the polypeptide sequence of SEQ ID NO: 67 and the polypeptide sequence of SEQ ID NO: 33, or variants thereof that retain functionality. In one embodiment the T cell activating bispecific antigen binding molecule comprises the polypeptide sequence of SEQ ID NO: 65, the polypeptide sequence of SEQ ID NO: 67, the polypeptide sequence of SEQ ID NO: 183 and the polypeptide sequence of SEQ ID NO: 197, or variants thereof that retain functionality. In one embodiment the T cell activating bispecific antigen binding molecule comprises the polypeptide sequence of SEQ ID NO: 183, the polypeptide sequence of SEQ ID NO: 203, the polypeptide sequence of SEQ ID NO: 205 and the polypeptide sequence of SEQ ID NO: 207, or variants thereof that retain functionality. In one embodiment the T cell activating bispecific antigen binding molecule comprises the polypeptide sequence of SEQ ID NO: 183, the polypeptide sequence of SEQ ID NO: 209, the polypeptide sequence of SEQ ID NO: 211 and the polypeptide sequence of SEQ ID NO: 213, or variants thereof that retain functionality.

In a specific embodiment the T cell activating bispecific antigen binding molecule comprises a polypeptide sequence encoded by a polynucleotide sequence that is at least about 80%, 85%, 90%, 95%, 96%, 97%, 98%, 99% or 100% identical to a sequence selected from the group of SEQ ID NO: 118, SEQ ID NO: 120, SEQ ID NO: 122, SEQ ID NO: 124, SEQ ID NO: 126, SEQ ID NO: 128, SEQ ID NO: 130, SEQ ID NO: 132, SEQ ID NO: 64, SEQ ID NO: 66, SEQ ID NO: 68, SEQ ID NO: 34, SEQ ID NO: 184, SEQ ID NO: 198, SEQ ID NO: 204, SEQ ID NO: 206, SEQ ID NO: 208, SEQ ID NO: 210, SEQ ID NO: 212 and SEQ ID NO: 214.

In one embodiment the T cell activating bispecific antigen binding molecule comprises at least one antigen binding moiety that is specific for CD33. In one embodiment, the antigen binding moiety that is specific for CD33 comprises the heavy chain CDR1 of SEQ ID NO: 133, the heavy chain CDR2 of SEQ ID NO: 135, the heavy chain CDR3 of SEQ ID NO: 137, the light chain CDR1 of SEQ ID NO: 141, the light chain CDR2 of SEQ ID NO: 143, and the light chain CDR3 of SEQ ID NO: 145. In a further embodiment, the antigen binding moiety that is specific for CD33 comprises a heavy chain variable region sequence that is at least about 80%, 85%, 90%, 95%, 96%, 97%, 98%, 99% or 100% identical to SEQ ID NO: 139 and a light chain variable region sequence that is at least about 80%, 85%, 90%, 95%, 96%, 97%, 98%, 99% or 100% identical to SEQ ID NO: 147, or variants thereof that retain functionality.

In one embodiment the T cell activating bispecific antigen binding molecule comprises the polypeptide sequence of SEQ ID NO: 33, the polypeptide sequence of SEQ ID NO: 213, the polypeptide sequence of SEQ ID NO: 221 and the polypeptide sequence of SEQ ID NO: 223, or variants thereof that retain functionality. In one embodiment the T cell activating bispecific antigen binding molecule comprises the polypeptide sequence of SEQ ID NO: 33, the polypeptide sequence of SEQ ID NO: 221, the polypeptide sequence of SEQ ID NO: 223 and the polypeptide sequence of SEQ ID NO: 225, or variants thereof that retain functionality.

In a specific embodiment the T cell activating bispecific antigen binding molecule comprises a polypeptide sequence encoded by a polynucleotide sequence that is at least about 80%, 85%, 90%, 95%, 96%, 97%, 98%, 99% or 100% identical to a sequence selected from the group of SEQ ID NO: 134, SEQ ID NO: 136, SEQ ID NO: 138, SEQ ID NO: 140, SEQ ID NO: 142, SEQ ID NO: 144, SEQ ID NO: 146, SEQ ID NO: 148, SEQ ID NO: 34, SEQ ID NO: 214, SEQ ID NO: 222, SEQ ID NO: 224 and SEQ ID NO: 226.

### Polynucleotides

The invention further provides isolated polynucleotides encoding a T cell activating bispecific antigen binding molecule as described herein or a fragment thereof.

Polynucleotides of the invention include those that are at least about 80%, 85%, 90%, 95%, 96%, 97%, 98%, 99%, or 100% identical to the sequences set forth in SEQ ID NOs 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262 and 264, including functional fragments or variants thereof.

The polynucleotides encoding T cell activating bispecific antigen binding molecules of the invention may be expressed as a single polynucleotide that encodes the entire T cell activating bispecific antigen binding molecule or as multiple (e.g., two or more) polynucleotides that are co-expressed. Polypeptides encoded by polynucleotides that are co-expressed may associate through, e.g., disulfide bonds or other means to form a functional T cell activating bispecific antigen binding molecule. For example, the light chain portion of an antigen binding moiety may be encoded by a separate polynucleotide from the portion of the T cell activating bispecific antigen binding molecule comprising the heavy chain portion of the antigen binding moiety, an Fc domain subunit and optionally (part of) another antigen binding moiety. When co-expressed, the heavy chain polypeptides will associate with the light chain polypeptides to form the antigen binding moiety. In another example, the portion of the T cell activating bispecific antigen binding molecule comprising one of the two Fc domain subunits and optionally (part of) one or more antigen binding moieties could be encoded by a separate polynucleotide from the portion of the T cell activating bispecific antigen binding molecule comprising the the other of the two Fc domain subunits and optionally (part of) an antigen binding moiety. When co-expressed, the Fc domain subunits will associate to form the Fc domain.

In certain embodiments, an isolated polynucleotide of the invention encodes a fragment of a T cell activating bispecific antigen binding molecule comprising a first and a second antigen binding moiety, and an Fc domain consisting of two subunits, wherein the first antigen binding moiety is a single chain Fab molecule. In one embodiment, an isolated polynucleotide of the invention encodes the first antigen binding moiety and a subunit of the Fc domain. In a more specific embodiment the isolated polynucleotide encodes a polypeptide wherein a single chain Fab molecule shares a carboxy-terminal peptide bond with an Fc domain subunit. In another embodiment, an isolated polynucleotide of the invention encodes the heavy chain of the second antigen binding moiety and a subunit of the Fc domain. In a more specific embodiment the isolated polynucleotide encodes a polypeptide wherein a Fab heavy chain shares a carboxy terminal peptide bond with an Fc domain subunit. In yet another embodiment, an isolated polynucleotide of the invention encodes the first antigen binding moiety, the heavy chain of the second antigen binding moiety and a subunit of the Fc domain. In a more specific embodiment, the isolated polynucleotide encodes a polypeptide wherein a single chain Fab molecule shares a carboxy-terminal peptide bond with a Fab heavy chain, which in turn shares a carboxy-terminal peptide bond with an Fc domain subunit.

In certain embodiments, an isolated polynucleotide of the invention encodes a fragment of a T cell activating bispecific antigen binding molecule comprising a first and a second antigen binding moiety, and an Fc domain consisting of two subunits, wherein the first antigen binding moiety is a crossover Fab molecule. In one embodiment, an isolated polynucleotide of the invention encodes the heavy chain of the first antigen binding moiety and a subunit of the Fc domain. In a more specific embodiment the isolated polynucleotide encodes a polypeptide wherein Fab light chain variable region shares a carboxy terminal peptide bond with a Fab heavy chain constant region, which in turn shares a carboxy-terminal peptide bond with an Fc domain subunit. In another specific embodiment the isolated polynucleotide encodes a polypeptide wherein Fab heavy chain variable region shares a carboxy terminal peptide bond with a Fab light chain constant region, which in turn shares a carboxy-terminal peptide bond with an Fc domain subunit. In another embodiment, an isolated polynucleotide of the invention encodes the heavy chain of the second antigen binding moiety and a subunit of the Fc domain. In a more specific embodiment the isolated polynucleotide encodes a polypeptide wherein a Fab heavy chain shares a carboxy terminal peptide bond with an Fc domain subunit. In yet another embodiment, an isolated polynucleotide of the invention encodes the heavy chain of the first antigen binding moiety, the heavy chain of the second antigen binding moiety and a subunit of the Fc domain. In a more specific embodiment, the isolated polynucleotide encodes a polypeptide wherein a Fab light chain variable region shares a carboxy-terminal peptide bond with a Fab heavy chain constant region, which in turn shares a carboxy-terminal peptide bond with a Fab heavy chain, which in turn shares a carboxy-terminal peptide bond with an Fc domain subunit. In another specific embodiment, the isolated polynucleotide encodes a polypeptide wherein a Fab heavy chain variable region shares a carboxy-terminal peptide bond with a Fab light chain constant region, which in turn shares a carboxy-terminal peptide bond with a Fab heavy chain, which in turn shares a carboxy-terminal peptide bond with an Fc domain subunit. In yet another specific embodiment the isolated polynucleotide encodes a polypeptide wherein a Fab heavy chain shares a carboxy-terminal peptide bond with a Fab light chain variable region, which in turn shares a carboxy-terminal peptide bond with a Fab heavy chain constant region, which in turn shares a carboxy-terminal peptide bond with an Fc domain subunit. In still another specific embodiment the isolated polynucleotide encodes a polypeptide wherein a Fab heavy chain shares a carboxy-terminal peptide bond with a Fab heavy chain variable region, which in turn shares a carboxy-terminal peptide bond with a Fab light chain constant region, which in turn shares a carboxy-terminal peptide bond with an Fc domain subunit.

In further embodiments, an isolated polynucleotide of the invention encodes the heavy chain of a third antigen binding moiety and a subunit of the Fc domain. In a more specific embodiment the isolated polynucleotide encodes a polypeptide wherein a Fab heavy chain shares a carboxy terminal peptide bond with an Fc domain subunit.

In further embodiments, an isolated polynucleotide of the invention encodes the light chain of an antigen binding moiety. In some embodiments, the isolated polynucleotide encodes a polypeptide wherein a Fab light chain variable region shares a carboxy-terminal peptide bond with a Fab heavy chain constant region. In other embodiments, the isolated polynucleotide encodes a polypeptide wherein a Fab heavy chain variable region shares a carboxy-terminal peptide bond with a Fab light chain constant region. In still other embodiments, an isolated polynucleotide of the invention encodes the light chain of the first antigen binding moiety and the light chain of the second antigen binding moiety. In a more specific embodiment, the isolated polynucleotide encodes a polypeptide wherein a Fab heavy chain variable region shares a carboxy-terminal peptide bond with a Fab light chain constant region, which in turn shares a carboxy-terminal peptide bond with a Fab light chain. In another specific embodiment the isolated polynucleotide encodes a polypeptide wherein a Fab light chain shares a carboxy-terminal peptide bond with a Fab heavy chain variable region, which in turn shares a carboxy-terminal peptide bond with a Fab light chain constant region. In yet another specific embodiment, the isolated polynucleotide encodes a polypeptide wherein a Fab light chain variable region shares a carboxy-terminal peptide bond with a Fab heavy chain constant region, which in turn shares a carboxy-terminal peptide bond with a Fab light chain. In yet another specific embodiment the isolated polynucleotide encodes a polypeptide wherein a Fab light chain shares a carboxy-terminal peptide bond with a Fab light chain variable region, which in turn shares a carboxy-terminal peptide bond with a Fab heavy chain constant region.

In another embodiment, the present invention is directed to an isolated polynucleotide encoding a T cell activating bispecific antigen binding molecule of the invention or a fragment thereof, wherein the polynucleotide comprises a sequence that encodes a variable region sequence as shown in SEQ ID NOs 75, 83, 91, 99, 107, 115, 123, 131, 139, 147, 169, 177, 239, 247, 255 and 263. In another embodiment, the present invention is directed to an isolated polynucleotide encoding a T cell activating bispecific antigen binding molecule or fragment thereof, wherein the polynucleotide comprises a sequence that encodes a polypeptide sequence as shown in SEQ ID NOs 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 179, 181, 183, 185, 187, 189, 191, 193, 195, 197, 199, 201, 203, 205, 207, 209, 211, 213, 215, 217, 219, 221, 223, 225, 227, 229 and 231. In another embodiment, the invention is further directed to an isolated polynucleotide encoding a T cell activating bispecific antigen binding molecule of the invention or a fragment thereof, wherein the polynucleotide comprises a sequence that is at least about 80%, 85%, 90%, 95%, 96%, 97%, 98%, or 99% identical to a nucleotide sequence shown in SEQ ID NOs 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262 or 264. In another embodiment, the invention is directed to an isolated polynucleotide encoding a T cell activating bispecific antigen binding molecule of the invention or a fragment thereof, wherein the polynucleotide comprises a nucleic acid sequence shown in SEQ ID NOs 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262 or 264. In another embodiment, the invention is directed to an isolated polynucleotide encoding a T cell activating bispecific antigen binding molecule of the invention or a fragment thereof, wherein the polynucleotide comprises a sequence that encodes a variable region sequence that is at least about 80%, 85%, 90%, 95%, 96%, 97%, 98%, or 99% identical to an amino acid sequence in SEQ ID NOs 75, 83, 91, 99, 107, 115, 123, 131, 139, 147, 169, 177, 239, 247, 255 or 263. In another embodiment, the invention is directed to an isolated polynucleotide encoding a T cell activating bispecific antigen binding molecule or fragment thereof, wherein the polynucleotide comprises a sequence that encodes a polypeptide sequence that is at least 80%, 85%, 90%, 95%, 96%, 97%, 98%, or 99% identical to an amino acid sequence in SEQ ID NOs 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 179, 181, 183, 185, 187, 189, 191, 193, 195, 197, 199, 201, 203, 205, 207, 209, 211, 213, 215, 217, 219, 221, 223, 225, 227, 229 or 231. The invention encompasses an isolated polynucleotide encoding a T cell activating bispecific antigen binding molecule of the invention or a fragment thereof, wherein the polynucleotide comprises a sequence that encodes the variable region sequence of SEQ ID NOs 75, 83, 91, 99, 107, 115, 123, 131, 139, 147, 169, 177, 239, 247, 255 or 263 with conservative amino acid substitutions. The invention also encompasses an isolated polynucleotide encoding a T cell activating bispecific antigen binding molecule of the invention or fragment thereof, wherein the polynucleotide comprises a sequence that encodes the polypeptide sequence of SEQ ID NOs 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 179, 181, 183, 185, 187, 189, 191, 193, 195, 197, 199, 201, 203, 205, 207, 209, 211, 213, 215, 217, 219, 221, 223, 225, 227, 229 or 231 with conservative amino acid substitutions.

In certain embodiments the polynucleotide or nucleic acid is DNA. In other embodiments, a polynucleotide of the present invention is RNA, for example, in the form of messenger RNA (mRNA). RNA of the present invention may be single stranded or double stranded.

### Recombinant Methods

T cell activating bispecific antigen binding molecules of the invention may be obtained, for example, by solid-state peptide synthesis (e.g. Merrifield solid phase synthesis) or recombinant production. For recombinant production one or more polynucleotide encoding the T cell activating bispecific antigen binding molecule (fragment), e.g., as described above, is isolated and inserted into one or more vectors for further cloning and/or expression in a host cell. Such polynucleotide may be readily isolated and sequenced using conventional procedures. In one embodiment a vector, preferably an expression vector, comprising one or more of the polynucleotides of the invention is provided. Methods which are well known to those skilled in the art can be used to construct expression vectors containing the coding sequence of a T cell activating bispecific antigen binding molecule (fragment) along with appropriate transcriptional/translational control signals. These methods include in vitro recombinant DNA techniques, synthetic techniques and in vivo recombination/genetic recombination. See, for example, the techniques described in Maniatis et al., MOLECULAR CLONING: A LABORATORY MANUAL, Cold Spring Harbor Laboratory, N.Y. (1989); and Ausubel et al., CURRENT PROTOCOLS IN MOLECULAR BIOLOGY, Greene Publishing Associates and Wiley Interscience, N.Y (1989). The expression vector can be part of a plasmid, virus, or may be a nucleic acid fragment. The expression vector includes an expression cassette into which the polynucleotide encoding the T cell activating bispecific antigen binding molecule (fragment) (i.e. the coding region) is cloned in operable association with a promoter and/or other transcription or translation control elements. As used herein, a “coding region” is a portion of nucleic acid which consists of codons translated into amino acids. Although a “stop codon” (TAG, TGA, or TAA) is not translated into an amino acid, it may be considered to be part of a coding region, if present, but any flanking sequences, for example promoters, ribosome binding sites, transcriptional terminators, introns, 5′ and 3′ untranslated regions, and the like, are not part of a coding region. Two or more coding regions can be present in a single polynucleotide construct, e.g. on a single vector, or in separate polynucleotide constructs, e.g. on separate (different) vectors. Furthermore, any vector may contain a single coding region, or may comprise two or more coding regions, e.g. a vector of the present invention may encode one or more polypeptides, which are post- or co-translationally separated into the final proteins via proteolytic cleavage. In addition, a vector, polynucleotide, or nucleic acid of the invention may encode heterologous coding regions, either fused or unfused to a polynucleotide encoding the T cell activating bispecific antigen binding molecule (fragment) of the invention, or variant or derivative thereof. Heterologous coding regions include without limitation specialized elements or motifs, such as a secretory signal peptide or a heterologous functional domain. An operable association is when a coding region for a gene product, e.g. a polypeptide, is associated with one or more regulatory sequences in such a way as to place expression of the gene product under the influence or control of the regulatory sequence(s). Two DNA fragments (such as a polypeptide coding region and a promoter associated therewith) are “operably associated” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operably associated with a nucleic acid encoding a polypeptide if the promoter was capable of effecting transcription of that nucleic acid. The promoter may be a cell-specific promoter that directs substantial transcription of the DNA only in predetermined cells. Other transcription control elements, besides a promoter, for example enhancers, operators, repressors, and transcription termination signals, can be operably associated with the polynucleotide to direct cell-specific transcription. Suitable promoters and other transcription control regions are disclosed herein. A variety of transcription control regions are known to those skilled in the art. These include, without limitation, transcription control regions, which function in vertebrate cells, such as, but not limited to, promoter and enhancer segments from cytomegaloviruses (e.g. the immediate early promoter, in conjunction with intron-A), simian virus 40 (e.g. the early promoter), and retroviruses (such as, e.g. Rous sarcoma virus). Other transcription control regions include those derived from vertebrate genes such as actin, heat shock protein, bovine growth hormone and rabbit â-globin, as well as other sequences capable of controlling gene expression in eukaryotic cells. Additional suitable transcription control regions include tissue-specific promoters and enhancers as well as inducible promoters (e.g. promoters inducible tetracyclins). Similarly, a variety of translation control elements are known to those of ordinary skill in the art. These include, but are not limited to ribosome binding sites, translation initiation and termination codons, and elements derived from viral systems (particularly an internal ribosome entry site, or IRES, also referred to as a CITE sequence). The expression cassette may also include other features such as an origin of replication, and/or chromosome integration elements such as retroviral long terminal repeats (LTRs), or adeno-associated viral (AAV) inverted terminal repeats (ITRs).

Polynucleotide and nucleic acid coding regions of the present invention may be associated with additional coding regions which encode secretory or signal peptides, which direct the secretion of a polypeptide encoded by a polynucleotide of the present invention. For example, if secretion of the T cell activating bispecific antigen binding molecule is desired, DNA encoding a signal sequence may be placed upstream of the nucleic acid encoding a T cell activating bispecific antigen binding molecule of the invention or a fragment thereof. According to the signal hypothesis, proteins secreted by mammalian cells have a signal peptide or secretory leader sequence which is cleaved from the mature protein once export of the growing protein chain across the rough endoplasmic reticulum has been initiated. Those of ordinary skill in the art are aware that polypeptides secreted by vertebrate cells generally have a signal peptide fused to the N-terminus of the polypeptide, which is cleaved from the translated polypeptide to produce a secreted or “mature” form of the polypeptide. In certain embodiments, the native signal peptide, e.g. an immunoglobulin heavy chain or light chain signal peptide is used, or a functional derivative of that sequence that retains the ability to direct the secretion of the polypeptide that is operably associated with it. Alternatively, a heterologous mammalian signal peptide, or a functional derivative thereof, may be used. For example, the wild-type leader sequence may be substituted with the leader sequence of human tissue plasminogen activator (TPA) or mouse β-glucuronidase. Exemplary amino acid and polynucleotide sequences of secretory signal peptides are given in SEQ ID NOs 154-162.

DNA encoding a short protein sequence that could be used to facilitate later purification (e.g. a histidine tag) or assist in labeling the T cell activating bispecific antigen binding molecule may be included within or at the ends of the T cell activating bispecific antigen binding molecule (fragment) encoding polynucleotide.

In a further embodiment, a host cell comprising one or more polynucleotides of the invention is provided. In certain embodiments a host cell comprising one or more vectors of the invention is provided. The polynucleotides and vectors may incorporate any of the features, singly or in combination, described herein in relation to polynucleotides and vectors, respectively. In one such embodiment a host cell comprises (e.g. has been transformed or transfected with) a vector comprising a polynucleotide that encodes (part of) a T cell activating bispecific antigen binding molecule of the invention. As used herein, the term “host cell” refers to any kind of cellular system which can be engineered to generate the T cell activating bispecific antigen binding molecules of the invention or fragments thereof. Host cells suitable for replicating and for supporting expression of T cell activating bispecific antigen binding molecules are well known in the art. Such cells may be transfected or transduced as appropriate with the particular expression vector and large quantities of vector containing cells can be grown for seeding large scale fermenters to obtain sufficient quantities of the T cell activating bispecific antigen binding molecule for clinical applications. Suitable host cells include prokaryotic microorganisms, such as E. coli, or various eukaryotic cells, such as Chinese hamster ovary cells (CHO), insect cells, or the like. For example, polypeptides may be produced in bacteria in particular when glycosylation is not needed. After expression, the polypeptide may be isolated from the bacterial cell paste in a soluble fraction and can be further purified. In addition to prokaryotes, eukaryotic microbes such as filamentous fungi or yeast are suitable cloning or expression hosts for polypeptide-encoding vectors, including fungi and yeast strains whose glycosylation pathways have been “humanized”, resulting in the production of a polypeptide with a partially or fully human glycosylation pattern. See Gerngross, Nat Biotech 22, 1409-1414 (2004), and Li et al., Nat Biotech 24, 210-215 (2006). Suitable host cells for the expression of (glycosylated) polypeptides are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells. Plant cell cultures can also be utilized as hosts. See e.g. U.S. Pat. Nos. 5,959,177, 6,040,498, 6,420,548, 7,125,978, and 6,417,429 (describing PLANTIBODIES™ technology for producing antibodies in transgenic plants). Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293T cells as described, e.g., in Graham et al., J Gen Virol 36, 59 (1977)), baby hamster kidney cells (BHK), mouse sertoli cells (TM4 cells as described, e.g., in Mather, Biol Reprod 23, 243-251 (1980)), monkey kidney cells (CV1), African green monkey kidney cells (VERO-76), human cervical carcinoma cells (HELA), canine kidney cells (MDCK), buffalo rat liver cells (BRL 3A), human lung cells (W138), human liver cells (Hep G2), mouse mammary tumor cells (MMT 060562), TRI cells (as described, e.g., in Mather et al., Annals N.Y. Acad Sci 383, 44-68 (1982)), MRC 5 cells, and FS4 cells. Other useful mammalian host cell lines include Chinese hamster ovary (CHO) cells, including dhfr⁻ CHO cells (Urlaub et al., Proc Natl Acad Sci USA 77, 4216 (1980)); and myeloma cell lines such as YO, NS0, P3X63 and Sp2/0. For a review of certain mammalian host cell lines suitable for protein production, see, e.g., Yazaki and Wu, Methods in Molecular Biology, Vol. 248 (B.K.C. Lo, ed., Humana Press, Totowa, NJ), pp. 255-268 (2003). Host cells include cultured cells, e.g., mammalian cultured cells, yeast cells, insect cells, bacterial cells and plant cells, to name only a few, but also cells comprised within a transgenic animal, transgenic plant or cultured plant or animal tissue. In one embodiment, the host cell is a eukaryotic cell, preferably a mammalian cell, such as a Chinese Hamster Ovary (CHO) cell, a human embryonic kidney (HEK) cell or a lymphoid cell (e.g., Y0, NS0, Sp20 cell).

Standard technologies are known in the art to express foreign genes in these systems. Cells expressing a polypeptide comprising either the heavy or the light chain of an antigen binding domain such as an antibody, may be engineered so as to also express the other of the antibody chains such that the expressed product is an antibody that has both a heavy and a light chain.

In one embodiment, a method of producing a T cell activating bispecific antigen binding molecule according to the invention is provided, wherein the method comprises culturing a host cell comprising a polynucleotide encoding the T cell activating bispecific antigen binding molecule, as provided herein, under conditions suitable for expression of the T cell activating bispecific antigen binding molecule, and recovering the T cell activating bispecific antigen binding molecule from the host cell (or host cell culture medium).

The components of the T cell activating bispecific antigen binding molecule are genetically fused to each other. T cell activating bispecific antigen binding molecule can be designed such that its components are fused directly to each other or indirectly through a linker sequence. The composition and length of the linker may be determined in accordance with methods well known in the art and may be tested for efficacy. Examples of linker sequences between different components of T cell activating bispecific antigen binding molecules are found in the sequences provided herein. Additional sequences may also be included to incorporate a cleavage site to separate the individual components of the fusion if desired, for example an endopeptidase recognition sequence.

In certain embodiments the one or more antigen binding moieties of the T cell activating bispecific antigen binding molecules comprise at least an antibody variable region capable of binding an antigenic determinant. Variable regions can form part of and be derived from naturally or non-naturally occurring antibodies and fragments thereof. Methods to produce polyclonal antibodies and monoclonal antibodies are well known in the art (see e.g. Harlow and Lane, “Antibodies, a laboratory manual”, Cold Spring Harbor Laboratory, 1988). Non-naturally occurring antibodies can be constructed using solid phase-peptide synthesis, can be produced recombinantly (e.g. as described in U.S. Pat. No. 4,186,567) or can be obtained, for example, by screening combinatorial libraries comprising variable heavy chains and variable light chains (see e.g. U.S. Pat. No. 5,969,108 to McCafferty).

Any animal species of antibody, antibody fragment, antigen binding domain or variable region can be used in the T cell activating bispecific antigen binding molecules of the invention. Non-limiting antibodies, antibody fragments, antigen binding domains or variable regions useful in the present invention can be of murine, primate, or human origin. If the T cell activating bispecific antigen binding molecule is intended for human use, a chimeric form of antibody may be used wherein the constant regions of the antibody are from a human. A humanized or fully human form of the antibody can also be prepared in accordance with methods well known in the art (see e. g. U.S. Pat. No. 5,565,332 to Winter). Humanization may be achieved by various methods including, but not limited to (a) grafting the non-human (e.g., donor antibody) CDRs onto human (e.g. recipient antibody) framework and constant regions with or without retention of critical framework residues (e.g. those that are important for retaining good antigen binding affinity or antibody functions), (b) grafting only the non-human specificity-determining regions (SDRs or a-CDRs; the residues critical for the antibody-antigen interaction) onto human framework and constant regions, or (c) transplanting the entire non-human variable domains, but “cloaking” them with a human-like section by replacement of surface residues. Humanized antibodies and methods of making them are reviewed, e.g., in Almagro and Fransson, Front Biosci 13, 1619-1633 (2008), and are further described, e.g., in Riechmann et al., Nature 332, 323-329 (1988); Queen et al., Proc Natl Acad Sci USA 86, 10029-10033 (1989); U.S. Pat. Nos. 5,821,337, 7,527,791, 6,982,321, and 7,087,409; Jones et al., Nature 321, 522-525 (1986); Morrison et al., Proc Natl Acad Sci 81, 6851-6855 (1984); Morrison and Oi, Adv Immunol 44, 65-92 (1988); Verhoeyen et al., Science 239, 1534-1536 (1988); Padlan, Molec Immun 31(3), 169-217 (1994); Kashmiri et al., Methods 36, 25-34 (2005) (describing SDR (a-CDR) grafting); Padlan, Mol Immunol 28, 489-498 (1991) (describing “resurfacing”); Dall’Acqua et al., Methods 36, 43-60 (2005) (describing “FR shuffling”); and Osbourn et al., Methods 36, 61-68 (2005) and Klimka et al., Br J Cancer 83, 252-260 (2000) (describing the “guided selection” approach to FR shuffling). Human antibodies and human variable regions can be produced using various techniques known in the art. Human antibodies are described generally in van Dijk and van de Winkel, Curr Opin Pharmacol 5, 368-74 (2001) and Lonberg, Curr Opin Immunol 20, 450-459 (2008). Human variable regions can form part of and be derived from human monoclonal antibodies made by the hybridoma method (see e.g. Monoclonal Antibody Production Techniques and Applications, pp. 51-63 (Marcel Dekker, Inc., New York, 1987)). Human antibodies and human variable regions may also be prepared by administering an immunogen to a transgenic animal that has been modified to produce intact human antibodies or intact antibodies with human variable regions in response to antigenic challenge (see e.g. Lonberg, Nat Biotech 23, 1117-1125 (2005). Human antibodies and human variable regions may also be generated by isolating Fv clone variable region sequences selected from human-derived phage display libraries (see e.g., Hoogenboom et al. in Methods in Molecular Biology 178, 1-37 (O′Brien et al., ed., Human Press, Totowa, NJ, 2001); and McCafferty et al., Nature 348, 552-554; Clackson et al., Nature 352, 624-628 (1991)). Phage typically display antibody fragments, either as single-chain Fv (scFv) fragments or as Fab fragments.

In certain embodiments, the antigen binding moieties useful in the present invention are engineered to have enhanced binding affinity according to, for example, the methods disclosed in U.S. Pat. Appl. Publ. No. 2004/0132066, the entire contents of which are hereby incorporated by reference. The ability of the T cell activating bispecific antigen binding molecule of the invention to bind to a specific antigenic determinant can be measured either through an enzyme-linked immunosorbent assay (ELISA) or other techniques familiar to one of skill in the art, e.g. surface plasmon resonance technique (analyzed on a BIACORE T100 system) (Liljeblad, et al., Glyco J 17, 323-329 (2000)), and traditional binding assays (Heeley, Endocr Res 28, 217-229 (2002)). Competition assays may be used to identify an antibody, antibody fragment, antigen binding domain or variable domain that competes with a reference antibody for binding to a particular antigen, e.g. an antibody that competes with the V9 antibody for binding to CD3. In certain embodiments, such a competing antibody binds to the same epitope (e.g. a linear or a conformational epitope) that is bound by the reference antibody. Detailed exemplary methods for mapping an epitope to which an antibody binds are provided in Morris (1996) “Epitope Mapping Protocols,” in Methods in Molecular Biology vol. 66 (Humana Press, Totowa, NJ). In an exemplary competition assay, immobilized antigen (e.g. CD3) is incubated in a solution comprising a first labeled antibody that binds to the antigen (e.g. V9 antibody) and a second unlabeled antibody that is being tested for its ability to compete with the first antibody for binding to the antigen. The second antibody may be present in a hybridoma supernatant. As a control, immobilized antigen is incubated in a solution comprising the first labeled antibody but not the second unlabeled antibody. After incubation under conditions permissive for binding of the first antibody to the antigen, excess unbound antibody is removed, and the amount of label associated with immobilized antigen is measured. If the amount of label associated with immobilized antigen is substantially reduced in the test sample relative to the control sample, then that indicates that the second antibody is competing with the first antibody for binding to the antigen. See Harlow and Lane (1988) Antibodies: A Laboratory Manual ch.14 (Cold Spring Harbor Laboratory, Cold Spring Harbor, NY).

T cell activating bispecific antigen binding molecules prepared as described herein may be purified by art-known techniques such as high performance liquid chromatography, ion exchange chromatography, gel electrophoresis, affinity chromatography, size exclusion chromatography, and the like. The actual conditions used to purify a particular protein will depend, in part, on factors such as net charge, hydrophobicity, hydrophilicity etc., and will be apparent to those having skill in the art. For affinity chromatography purification an antibody, ligand, receptor or antigen can be used to which the T cell activating bispecific antigen binding molecule binds. For example, for affinity chromatography purification of T cell activating bispecific antigen binding molecules of the invention, a matrix with protein A or protein G may be used. Sequential Protein A or G affinity chromatography and size exclusion chromatography can be used to isolate a T cell activating bispecific antigen binding molecule essentially as described in the Examples. The purity of the T cell activating bispecific antigen binding molecule can be determined by any of a variety of well known analytical methods including gel electrophoresis, high pressure liquid chromatography, and the like. For example, the heavy chain fusion proteins expressed as described in the Examples were shown to be intact and properly assembled as demonstrated by reducing SDS-PAGE (see e.g. FIG. 2A - FIG. 2D). Three bands were resolved at approximately Mr 25,000, Mr 50,000 and Mr 75,000, corresponding to the predicted molecular weights of the T cell activating bispecific antigen binding molecule light chain, heavy chain and heavy chain/light chain fusion protein.

### Assays

T cell activating bispecific antigen binding molecules provided herein may be identified, screened for, or characterized for their physical/chemical properties and/or biological activities by various assays known in the art.

### Affinity Assays

The affinity of the T cell activating bispecific antigen binding molecule for an Fc receptor or a target antigen can be determined in accordance with the methods set forth in the Examples by surface plasmon resonance (SPR), using standard instrumentation such as a BIAcore instrument (GE Healthcare), and receptors or target proteins such as may be obtained by recombinant expression. Alternatively, binding of T cell activating bispecific antigen binding molecules for different receptors or target antigens may be evaluated using cell lines expressing the particular receptor or target antigen, for example by flow cytometry (FACS). A specific illustrative and exemplary embodiment for measuring binding affinity is described in the following and in the Examples below. According to one embodiment, K_{D} is measured by surface plasmon resonance using a BIACORE® T100 machine (GE Healthcare) at 25° C.

To analyze the interaction between the Fc-portion and Fc receptors, His-tagged recombinant Fc-receptor is captured by an anti-Penta His antibody (Qiagen) immobilized on CM5 chips and the bispecific constructs are used as analytes. Briefly, carboxymethylated dextran biosensor chips (CM5, GE Healthcare) are activated with N-ethyl-N′-(3-dimethylaminopropyl)-carbodiimide hydrochloride (EDC) and N-hydroxysuccinimide (NHS) according to the supplier’s instructions. Anti Penta-His antibody is diluted with 10 mM sodium acetate, pH 5.0, to 40 µg/ml before injection at a flow rate of 5 µl/min to achieve approximately 6500 response units (RU) of coupled protein. Following the injection of the ligand, 1 M ethanolamine is injected to block unreacted groups. Subsequently the Fc-receptor is captured for 60 s at 4 or 10 nM. For kinetic measurements, four-fold serial dilutions of the bispecific construct (range between 500 nM and 4000 nM) are injected in HBS-EP (GE Healthcare, 10 mM HEPES, 150 mM NaCl, 3 mM EDTA, 0.05 % Surfactant P20, pH 7.4) at 25° C. at a flow rate of 30 µl/min for 120 s.

To determine the affinity to the target antigen, bispecific constructs are captured by an anti human Fab specific antibody (GE Healthcare) that is immobilized on an activated CM5-sensor chip surface as described for the anti Penta-His antibody. The final amount of coupled protein is is approximately 12000 RU. The bispecific constructs are captured for 90 s at 300 nM. The target antigens are passed through the flow cells for 180 s at a concentration range from 250 to 1000 nM with a flowrate of 30 µl/min. The dissociation is monitored for 180 s.

Bulk refractive index differences are corrected for by subtracting the response obtained on reference flow cell. The steady state response was used to derive the dissociation constant K_{D} by non-linear curve fitting of the Langmuir binding isotherm. Association rates (kₒₙ) and dissociation rates (k_{off}) are calculated using a simple one-to-one Langmuir binding model (BIACORE® T100 Evaluation Software version 1.1.1) by simultaneously fitting the association and dissociation sensorgrams. The equilibrium dissociation constant (K_{D}) is calculated as the ratio k_{off}/kₒₙ. See, e.g., Chen et al., J Mol Biol 293, 865-881 (1999).

### Activity Assays

Biological activity of the T cell activating bispecific antigen binding molecules of the invention can be measured by various assays as described in the Examples. Biological activities may for example include the induction of proliferation of T cells, the induction of signaling in T cells, the induction of expression of activation markers in T cells, the induction of cytokine secretion by T cells, the induction of lysis of target cells such as tumor cells, and the induction of tumor regression and/or the improvement of survival.

### Compositions, Formulations, and Routes of Administration

In a further aspect, the invention provides pharmaceutical compositions comprising any of the T cell activating bispecific antigen binding molecules provided herein, e.g., for use in any of the below therapeutic methods. In one embodiment, a pharmaceutical composition comprises any of the T cell activating bispecific antigen binding molecules provided herein and a pharmaceutically acceptable carrier. In another embodiment, a pharmaceutical composition comprises any of the T cell activating bispecific antigen binding molecules provided herein and at least one additional therapeutic agent, e.g., as described below.

Further provided is a method of producing a T cell activating bispecific antigen binding molecule of the invention in a form suitable for administration in vivo, the method comprising (a) obtaining a T cell activating bispecific antigen binding molecule according to the invention, and (b) formulating the T cell activating bispecific antigen binding molecule with at least one pharmaceutically acceptable carrier, whereby a preparation of T cell activating bispecific antigen binding molecule is formulated for administration in vivo.

Pharmaceutical compositions of the present invention comprise a therapeutically effective amount of one or more T cell activating bispecific antigen binding molecule dissolved or dispersed in a pharmaceutically acceptable carrier. The phrases “pharmaceutical or pharmacologically acceptable” refers to molecular entities and compositions that are generally non-toxic to recipients at the dosages and concentrations employed, i.e. do not produce an adverse, allergic or other untoward reaction when administered to an animal, such as, for example, a human, as appropriate. The preparation of a pharmaceutical composition that contains at least one T cell activating bispecific antigen binding molecule and optionally an additional active ingredient will be known to those of skill in the art in light of the present disclosure, as exemplified by Remington’s Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards or corresponding authorities in other countries. Preferred compositions are lyophilized formulations or aqueous solutions. As used herein, “pharmaceutically acceptable carrier” includes any and all solvents, buffers, dispersion media, coatings, surfactants, antioxidants, preservatives (e.g. antibacterial agents, antifungal agents), isotonic agents, absorption delaying agents, salts, preservatives, antioxidants, proteins, drugs, drug stabilizers, polymers, gels, binders, excipients, disintegration agents, lubricants, sweetening agents, flavoring agents, dyes, such like materials and combinations thereof, as would be known to one of ordinary skill in the art (see, for example, Remington’s Pharmaceutical Sciences, 18th Ed. Mack Printing Company, 1990, pp. 1289-1329, incorporated herein by reference). Except insofar as any conventional carrier is incompatible with the active ingredient, its use in the therapeutic or pharmaceutical compositions is contemplated.

The composition may comprise different types of carriers depending on whether it is to be administered in solid, liquid or aerosol form, and whether it need to be sterile for such routes of administration as injection. T cell activating bispecific antigen binding molecules of the present invention (and any additional therapeutic agent) can be administered intravenously, intradermally, intraarterially, intraperitoneally, intralesionally, intracranially, intraarticularly, intraprostatically, intrasplenically, intrarenally, intrapleurally, intratracheally, intranasally, intravitreally, intravaginally, intrarectally, intratumorally, intramuscularly, intraperitoneally, subcutaneously, subconjunctivally, intravesicularlly, mucosally, intrapericardially, intraumbilically, intraocularally, orally, topically, locally, by inhalation (e.g. aerosol inhalation), injection, infusion, continuous infusion, localized perfusion bathing target cells directly, via a catheter, via a lavage, in cremes, in lipid compositions (e.g. liposomes), or by other method or any combination of the forgoing as would be known to one of ordinary skill in the art (see, for example, Remington’s Pharmaceutical Sciences, 18th Ed. Mack Printing Company, 1990, incorporated herein by reference). Parenteral administration, in particular intravenous injection, is most commonly used for administering polypeptide molecules such as the T cell activating bispecific antigen binding molecules of the invention.

Parenteral compositions include those designed for administration by injection, e.g. subcutaneous, intradermal, intralesional, intravenous, intraarterial intramuscular, intrathecal or intraperitoneal injection. For injection, the T cell activating bispecific antigen binding molecules of the invention may be formulated in aqueous solutions, preferably in physiologically compatible buffers such as Hanks’ solution, Ringer’s solution, or physiological saline buffer. The solution may contain formulatory agents such as suspending, stabilizing and/or dispersing agents. Alternatively, the T cell activating bispecific antigen binding molecules may be in powder form for constitution with a suitable vehicle, e.g., sterile pyrogen-free water, before use. Sterile injectable solutions are prepared by incorporating the T cell activating bispecific antigen binding molecules of the invention in the required amount in the appropriate solvent with various of the other ingredients enumerated below, as required. Sterility may be readily accomplished, e.g., by filtration through sterile filtration membranes. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The composition must be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein. Suitable pharmaceutically acceptable carriers include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Aqueous injection suspensions may contain compounds which increase the viscosity of the suspension, such as sodium carboxymethyl cellulose, sorbitol, dextran, or the like. Optionally, the suspension may also contain suitable stabilizers or agents which increase the solubility of the compounds to allow for the preparation of highly concentrated solutions. Additionally, suspensions of the active compounds may be prepared as appropriate oily injection suspensions. Suitable lipophilic solvents or vehicles include fatty oils such as sesame oil, or synthetic fatty acid esters, such as ethyl cleats or triglycerides, or liposomes.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington’s Pharmaceutical Sciences (18th Ed. Mack Printing Company, 1990). Sustained-release preparations may be prepared. Suitable examples of sustained-release preparations include semipermeable matrices of solid hydrophobic polymers containing the polypeptide, which matrices are in the form of shaped articles, e.g. films, or microcapsules. In particular embodiments, prolonged absorption of an injectable composition can be brought about by the use in the compositions of agents delaying absorption, such as, for example, aluminum monostearate, gelatin or combinations thereof.

In addition to the compositions described previously, the T cell activating bispecific antigen binding molecules may also be formulated as a depot preparation. Such long acting formulations may be administered by implantation (for example subcutaneously or intramuscularly) or by intramuscular injection. Thus, for example, the T cell activating bispecific antigen binding molecules may be formulated with suitable polymeric or hydrophobic materials (for example as an emulsion in an acceptable oil) or ion exchange resins, or as sparingly soluble derivatives, for example, as a sparingly soluble salt.

Pharmaceutical compositions comprising the T cell activating bispecific antigen binding molecules of the invention may be manufactured by means of conventional mixing, dissolving, emulsifying, encapsulating, entrapping or lyophilizing processes. Pharmaceutical compositions may be formulated in conventional manner using one or more physiologically acceptable carriers, diluents, excipients or auxiliaries which facilitate processing of the proteins into preparations that can be used pharmaceutically. Proper formulation is dependent upon the route of administration chosen.

The T cell activating bispecific antigen binding molecules may be formulated into a composition in a free acid or base, neutral or salt form. Pharmaceutically acceptable salts are salts that substantially retain the biological activity of the free acid or base. These include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Pharmaceutical salts tend to be more soluble in aqueous and other protic solvents than are the corresponding free base forms.

### Therapeutic Methods and Compositions

Any of the T cell activating bispecific antigen binding molecules provided herein may be used in therapeutic methods. T cell activating bispecific antigen binding molecules of the invention can be used as immunotherapeutic agents, for example in the treatment of cancers.

For use in therapeutic methods, T cell activating bispecific antigen binding molecules of the invention would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

In one aspect, T cell activating bispecific antigen binding molecules of the invention for use as a medicament are provided. In further aspects, T cell activating bispecific antigen binding molecules of the invention for use in treating a disease are provided. In certain embodiments, T cell activating bispecific antigen binding molecules of the invention for use in a method of treatment are provided. In one embodiment, the invention provides a T cell activating bispecific antigen binding molecule as described herein for use in the treatment of a disease in an individual in need thereof. In certain embodiments, the invention provides a T cell activating bispecific antigen binding molecule for use in a method of treating an individual having a disease comprising administering to the individual a therapeutically effective amount of the T cell activating bispecific antigen binding molecule. In certain embodiments the disease to be treated is a proliferative disorder. In a particular embodiment the disease is cancer. In certain embodiments the method further comprises administering to the individual a therapeutically effective amount of at least one additional therapeutic agent, e.g., an anti-cancer agent if the disease to be treated is cancer. In further embodiments, the invention provides a T cell activating bispecific antigen binding molecule as described herein for use in inducing lysis of a target cell, particularly a tumor cell. In certain embodiments, the invention provides a T cell activating bispecific antigen binding molecule for use in a method of inducing lysis of a target cell, particularly a tumor cell, in an individual comprising administering to the individual an effective amount of the T cell activating bispecific antigen binding molecule to induce lysis of a target cell. An “individual” according to any of the above embodiments is a mammal, preferably a human.

In a further aspect, the invention provides for the use of a T cell activating bispecific antigen binding molecule of the invention in the manufacture or preparation of a medicament. In one embodiment the medicament is for the treatment of a disease in an individual in need thereof. In a further embodiment, the medicament is for use in a method of treating a disease comprising administering to an individual having the disease a therapeutically effective amount of the medicament. In certain embodiments the disease to be treated is a proliferative disorder. In a particular embodiment the disease is cancer. In one embodiment, the method further comprises administering to the individual a therapeutically effective amount of at least one additional therapeutic agent, e.g., an anti-cancer agent if the disease to be treated is cancer. In a further embodiment, the medicament is for inducing lysis of a target cell, particularly a tumor cell. In still a further embodiment, the medicament is for use in a method of inducing lysis of a target cell, particularly a tumor cell, in an individual comprising administering to the individual an effective amount of the medicament to induce lysis of a target cell. An “individual” according to any of the above embodiments may be a mammal, preferably a human.

In a further aspect, the invention provides a method for treating a disease. In one embodiment, the method comprises administering to an individual having such disease a therapeutically effective amount of a T cell activating bispecific antigen binding molecule of the invention. In one embodiment a composition is administered to said invididual, comprising the T cell activating bispecific antigen binding molecule of the invention in a pharmaceutically acceptable form. In certain embodiments the disease to be treated is a proliferative disorder. In a particular embodiment the disease is cancer. In certain embodiments the method further comprises administering to the individual a therapeutically effective amount of at least one additional therapeutic agent, e.g., an anti-cancer agent if the disease to be treated is cancer. An “individual” according to any of the above embodiments may be a mammal, preferably a human.

In a further aspect, the invention provides a method for inducing lysis of a target cell, particularly a tumor cell. In one embodiment the method comprises contacting a target cell with a T cell activating bispecific antigen binding molecule of the invention in the presence of a T cell, particularly a cytotoxic T cell. In a further aspect, a method for inducing lysis of a target cell, particularly a tumor cell, in an individual is provided. In one such embodiment, the method comprises administering to the individual an effective amount of a T cell activating bispecific antigen binding molecule to induce lysis of a target cell. In one embodiment, an “individual” is a human.

In certain embodiments the disease to be treated is a proliferative disorder, particularly cancer. Non-limiting examples of cancers include bladder cancer, brain cancer, head and neck cancer, pancreatic cancer, lung cancer, breast cancer, ovarian cancer, uterine cancer, cervical cancer, endometrial cancer, esophageal cancer, colon cancer, colorectal cancer, rectal cancer, gastric cancer, prostate cancer, blood cancer, skin cancer, squamous cell carcinoma, bone cancer, and kidney cancer. Other cell proliferation disorders that can be treated using a T cell activating bispecific antigen binding molecule of the present invention include, but are not limited to neoplasms located in the: abdomen, bone, breast, digestive system, liver, pancreas, peritoneum, endocrine glands (adrenal, parathyroid, pituitary, testicles, ovary, thymus, thyroid), eye, head and neck, nervous system (central and peripheral), lymphatic system, pelvic, skin, soft tissue, spleen, thoracic region, and urogenital system. Also included are pre-cancerous conditions or lesions and cancer metastases. In certain embodiments the cancer is chosen from the group consisting of renal cell cancer, skin cancer, lung cancer, colorectal cancer, breast cancer, brain cancer, head and neck cancer. A skilled artisan readily recognizes that in many cases the T cell activating bispecific antigen binding molecule may not provide a cure but may only provide partial benefit. In some embodiments, a physiological change having some benefit is also considered therapeutically beneficial. Thus, in some embodiments, an amount of T cell activating bispecific antigen binding molecule that provides a physiological change is considered an “effective amount” or a “therapeutically effective amount”. The subject, patient, or individual in need of treatment is typically a mammal, more specifically a human.

In some embodiments, an effective amount of a T cell activating bispecific antigen binding molecule of the invention is administered to a cell. In other embodiments, a therapeutically effective amount of a T cell activating bispecific antigen binding molecule of the invention is administered to an individual for the treatment of disease.

For the prevention or treatment of disease, the appropriate dosage of a T cell activating bispecific antigen binding molecule of the invention (when used alone or in combination with one or more other additional therapeutic agents) will depend on the type of disease to be treated, the route of administration, the body weight of the patient, the type of T cell activating bispecific antigen binding molecule, the severity and course of the disease, whether the T cell activating bispecific antigen binding molecule is administered for preventive or therapeutic purposes, previous or concurrent therapeutic interventions, the patient’s clinical history and response to the T cell activating bispecific antigen binding molecule, and the discretion of the attending physician. The practitioner responsible for administration will, in any event, determine the concentration of active ingredient(s) in a composition and appropriate dose(s) for the individual subject. Various dosing schedules including but not limited to single or multiple administrations over various time-points, bolus administration, and pulse infusion are contemplated herein.

The T cell activating bispecific antigen binding molecule is suitably administered to the patient at one time or over a series of treatments. Depending on the type and severity of the disease, about 1 µg/kg to 15 mg/kg (e.g. 0.1 mg/kg - 10 mg/kg) of T cell activating bispecific antigen binding molecule can be an initial candidate dosage for administration to the patient, whether, for example, by one or more separate administrations, or by continuous infusion. One typical daily dosage might range from about 1 µg/kg to 100 mg/kg or more, depending on the factors mentioned above. For repeated administrations over several days or longer, depending on the condition, the treatment would generally be sustained until a desired suppression of disease symptoms occurs. One exemplary dosage of the T cell activating bispecific antigen binding molecule would be in the range from about 0.005 mg/kg to about 10 mg/kg. In other non-limiting examples, a dose may also comprise from about 1 microgram/kg body weight, about 5 microgram/kg body weight, about 10 microgram/kg body weight, about 50 microgram/kg body weight, about 100 microgram/kg body weight, about 200 microgram/kg body weight, about 350 microgram/kg body weight, about 500 microgram/kg body weight, about 1 milligram/kg body weight, about 5 milligram/kg body weight, about 10 milligram/kg body weight, about 50 milligram/kg body weight, about 100 milligram/kg body weight, about 200 milligram/kg body weight, about 350 milligram/kg body weight, about 500 milligram/kg body weight, to about 1000 mg/kg body weight or more per administration, and any range derivable therein. In non-limiting examples of a derivable range from the numbers listed herein, a range of about 5 mg/kg body weight to about 100 mg/kg body weight, about 5 microgram/kg body weight to about 500 milligram/kg body weight, etc., can be administered, based on the numbers described above. Thus, one or more doses of about 0.5 mg/kg, 2.0 mg/kg, 5.0 mg/kg or 10 mg/kg (or any combination thereof) may be administered to the patient. Such doses may be administered intermittently, e.g. every week or every three weeks (e.g. such that the patient receives from about two to about twenty, or e.g. about six doses of the T cell activating bispecific antigen binding molecule). An initial higher loading dose, followed by one or more lower doses may be administered. However, other dosage regimens may be useful. The progress of this therapy is easily monitored by conventional techniques and assays.

The T cell activating bispecific antigen binding molecules of the invention will generally be used in an amount effective to achieve the intended purpose. For use to treat or prevent a disease condition, the T cell activating bispecific antigen binding molecules of the invention, or pharmaceutical compositions thereof, are administered or applied in a therapeutically effective amount. Determination of a therapeutically effective amount is well within the capabilities of those skilled in the art, especially in light of the detailed disclosure provided herein.

For systemic administration, a therapeutically effective dose can be estimated initially from in vitro assays, such as cell culture assays. A dose can then be formulated in animal models to achieve a circulating concentration range that includes the IC₅₀ as determined in cell culture. Such information can be used to more accurately determine useful doses in humans.

Initial dosages can also be estimated from in vivo data, e.g., animal models, using techniques that are well known in the art. One having ordinary skill in the art could readily optimize administration to humans based on animal data.

Dosage amount and interval may be adjusted individually to provide plasma levels of the T cell activating bispecific antigen binding molecules which are sufficient to maintain therapeutic effect. Usual patient dosages for administration by injection range from about 0.1 to 50 mg/kg/day, typically from about 0.5 to 1 mg/kg/day. Therapeutically effective plasma levels may be achieved by administering multiple doses each day. Levels in plasma may be measured, for example, by HPLC.

In cases of local administration or selective uptake, the effective local concentration of the T cell activating bispecific antigen binding molecules may not be related to plasma concentration. One having skill in the art will be able to optimize therapeutically effective local dosages without undue experimentation.

A therapeutically effective dose of the T cell activating bispecific antigen binding molecules described herein will generally provide therapeutic benefit without causing substantial toxicity. Toxicity and therapeutic efficacy of a T cell activating bispecific antigen binding molecule can be determined by standard pharmaceutical procedures in cell culture or experimental animals. Cell culture assays and animal studies can be used to determine the LD₅₀ (the dose lethal to 50% of a population) and the ED₅₀ (the dose therapeutically effective in 50% of a population). The dose ratio between toxic and therapeutic effects is the therapeutic index, which can be expressed as the ratio LD₅₀/ED₅₀. T cell activating bispecific antigen binding molecules that exhibit large therapeutic indices are preferred. In one embodiment, the T cell activating bispecific antigen binding molecule according to the present invention exhibits a high therapeutic index. The data obtained from cell culture assays and animal studies can be used in formulating a range of dosages suitable for use in humans. The dosage lies preferably within a range of circulating concentrations that include the ED₅₀ with little or no toxicity. The dosage may vary within this range depending upon a variety of factors, e.g., the dosage form employed, the route of administration utilized, the condition of the subject, and the like. The exact formulation, route of administration and dosage can be chosen by the individual physician in view of the patient’s condition (see, e.g., Fingl et al., 1975, in: The Pharmacological Basis of Therapeutics, Ch. 1, p. 1, incorporated herein by reference in its entirety). The attending physician for patients treated with T cell activating bispecific antigen binding molecules of the invention would know how and when to terminate, interrupt, or adjust administration due to toxicity, organ dysfunction, and the like. Conversely, the attending physician would also know to adjust treatment to higher levels if the clinical response were not adequate (precluding toxicity). The magnitude of an administered dose in the management of the disorder of interest will vary with the severity of the condition to be treated, with the route of administration, and the like. The severity of the condition may, for example, be evaluated, in part, by standard prognostic evaluation methods. Further, the dose and perhaps dose frequency will also vary according to the age, body weight, and response of the individual patient.

### Other Agents and Treatments

The T cell activating bispecific antigen binding molecules of the invention may be administered in combination with one or more other agents in therapy. For instance, a T cell activating bispecific antigen binding molecule of the invention may be co-administered with at least one additional therapeutic agent. The term “therapeutic agent” encompasses any agent administered to treat a symptom or disease in an individual in need of such treatment. Such additional therapeutic agent may comprise any active ingredients suitable for the particular indication being treated, preferably those with complementary activities that do not adversely affect each other. In certain embodiments, an additional therapeutic agent is an immunomodulatory agent, a cytostatic agent, an inhibitor of cell adhesion, a cytotoxic agent, an activator of cell apoptosis, or an agent that increases the sensitivity of cells to apoptotic inducers. In a particular embodiment, the additional therapeutic agent is an anti-cancer agent, for example a microtubule disruptor, an antimetabolite, a topoisomerase inhibitor, a DNA intercalator, an alkylating agent, a hormonal therapy, a kinase inhibitor, a receptor antagonist, an activator of tumor cell apoptosis, or an antiangiogenic agent.

Such other agents are suitably present in combination in amounts that are effective for the purpose intended. The effective amount of such other agents depends on the amount of T cell activating bispecific antigen binding molecule used, the type of disorder or treatment, and other factors discussed above. The T cell activating bispecific antigen binding molecules are generally used in the same dosages and with administration routes as described herein, or about from 1 to 99% of the dosages described herein, or in any dosage and by any route that is empirically/clinically determined to be appropriate.

Such combination therapies noted above encompass combined administration (where two or more therapeutic agents are included in the same or separate compositions), and separate administration, in which case, administration of the T cell activating bispecific antigen binding molecule of the invention can occur prior to, simultaneously, and/or following, administration of the additional therapeutic agent and/or adjuvant. T cell activating bispecific antigen binding molecules of the invention can also be used in combination with radiation therapy.

### Articles of Manufacture

In another aspect of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is a T cell activating bispecific antigen binding molecule of the invention. The label or package insert indicates that the composition is used for treating the condition of choice. Moreover, the article of manufacture may comprise (a) a first container with a composition contained therein, wherein the composition comprises a T cell activating bispecific antigen binding molecule of the invention; and (b) a second container with a composition contained therein, wherein the composition comprises a further cytotoxic or otherwise therapeutic agent. The article of manufacture in this embodiment of the invention may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer’s solution and dextrose solution. It may further include other materials desirable from a commercial and user standpoint, including other buffers, diluents, filters, needles, and syringes.

### EXAMPLES

The following are examples of methods and compositions of the invention. It is understood that various other embodiments may be practiced, given the general description provided above.

### General Methods

### Recombinant DNA Techniques

Standard methods were used to manipulate DNA as described in Sambrook et al., Molecular cloning: A laboratory manual; Cold Spring Harbor Laboratory Press, Cold Spring Harbor, New York, 1989. The molecular biological reagents were used according to the manufacturers’ instructions. General information regarding the nucleotide sequences of human immunoglobulins light and heavy chains is given in: Kabat, E.A. et al., (1991) Sequences of Proteins of Immunological Interest, 5^{th} ed., NIH Publication No. 91-3242.

### DNA Sequencing

DNA sequences were determined by double strand sequencing.

### Gene Synthesis

Desired gene segments where required were either generated by PCR using appropriate templates or were synthesized by Geneart AG (Regensburg, Germany) from synthetic oligonucleotides and PCR products by automated gene synthesis. In cases where no exact gene sequence was available, oligonucleotide primers were designed based on sequences from closest homologues and the genes were isolated by RT-PCR from RNA originating from the appropriate tissue. The gene segments flanked by singular restriction endonuclease cleavage sites were cloned into standard cloning / sequencing vectors. The plasmid DNA was purified from transformed bacteria and concentration determined by UV spectroscopy. The DNA sequence of the subcloned gene fragments was confirmed by DNA sequencing. Gene segments were designed with suitable restriction sites to allow sub-cloning into the respective expression vectors. All constructs were designed with a 5′-end DNA sequence coding for a leader peptide which targets proteins for secretion in eukaryotic cells. SEQ ID NOs 154-162 give exemplary leader peptides and polynucleotide sequences encoding them, respectively.

### Isolation of Primary Human Pan T Cells From PBMCs

Peripheral blood mononuclear cells (PBMCs) were prepared by Histopaque density centrifugation from enriched lymphocyte preparations (buffy coats) obtained from local blood banks or from fresh blood from healthy human donors. Briefly, blood was diluted with sterile PBS and carefully layered over a Histopaque gradient (Sigma, H8889). After centrifugation for 30 minutes at 450 × g at room temperature (brake switched off), part of the plasma above the PBMC containing interphase was discarded. The PBMCs were transferred into new 50 ml Falcon tubes and tubes were filled up with PBS to a total volume of 50 ml. The mixture was centrifuged at room temperature for 10 minutes at 400 × g (brake switched on). The supernatant was discarded and the PBMC pellet washed twice with sterile PBS (centrifugation steps at 4° C. for 10 minutes at 350 × g). The resulting PBMC population was counted automatically (ViCell) and stored in RPMI1640 medium, containing 10% FCS and 1% L-alanyl-L-glutamine (Biochrom, K0302) at 37° C., 5% CO₂ in the incubator until assay start.

T cell enrichment from PBMCs was performed using the Pan T Cell Isolation Kit II (Miltenyi Biotec #130-091-156), according to the manufacturer’s instructions. Briefly, the cell pellets were diluted in 40 µl cold buffer per 10 million cells (PBS with 0.5% BSA, 2 mM EDTA, sterile filtered) and incubated with 10 µl Biotin-Antibody Cocktail per 10 million cells for 10 min at 4° C. 30 µl cold buffer and 20 µl Anti-Biotin magnetic beads per 10 million cells were added, and the mixture incubated for another 15 min at 4° C. Cells were washed by adding 10-20x the current volume and a subsequent centrifugation step at 300 × g for 10 min. Up to 100 million cells were resuspended in 500 µl buffer. Magnetic separation of unlabeled human pan T cells was performed using LS columns (Miltenyi Biotec #130-042-401) according to the manufacturer’s instructions. The resulting T cell population was counted automatically (ViCell) and stored in AIM-V medium at 37° C., 5% CO₂ in the incubator until assay start (not longer than 24 h).

### Isolation of Primary Human Naive T Cells From PBMCs

Peripheral blood mononuclar cells (PBMCs) were prepared by Histopaque density centrifugation from enriched lymphocyte preparations (buffy coats) obtained from local blood banks or from fresh blood from healthy human donors. T-cell enrichment from PBMCs was performed using the Naive CD8⁺ T cell isolation Kit from Miltenyi Biotec (#130-093-244), according to the manufacturer’s instructions, but skipping the last isolation step of CD8⁺ T cells (also see description for the isolation of primary human pan T cells).

### Isolation of Murine Pan T Cells From Splenocytes

Spleens were isolated from C57BL/6 mice, transferred into a GentleMACS C-tube (Miltenyi Biotech #130-093-237) containing MACS buffer (PBS + 0.5% BSA + 2 mM EDTA) and dissociated with the GentleMACS Dissociator to obtain single-cell suspensions according to the manufacturer’s instructions. The cell suspension was passed through a pre-separation filter to remove remaining undissociated tissue particles. After centrifugation at 400 × g for 4 min at 4° C., ACK Lysis Buffer was added to lyse red blood cells (incubation for 5 min at room temperature). The remaining cells were washed with MACS buffer twice, counted and used for the isolation of murine pan T cells. The negative (magnetic) selection was performed using the Pan T Cell Isolation Kit from Miltenyi Biotec (#130-090-861), following the manufacturer’s instructions. The resulting T cell population was automatically counted (ViCell) and immediately used for further assays.

### Isolation of Primary Cynomolgus PBMCs From Heparinized Blood

Peripheral blood mononuclar cells (PBMCs) were prepared by density centrifugation from fresh blood from healthy cynomolgus donors, as follows: Heparinized blood was diluted 1:3 with sterile PBS, and Lymphoprep medium (Axon Lab #1114545) was diluted to 90% with sterile PBS. Two volumes of the diluted blood were layered over one volume of the diluted density gradient and the PBMC fraction was separated by centrifugation for 30 min at 520 × g, without brake, at room temperature. The PBMC band was transferred into a fresh 50 ml Falcon tube and washed with sterile PBS by centrifugation for 10 min at 400 × g at 4° C. One low-speed centrifugation was performed to remove the platelets (15 min at 150 × g, 4° C.), and the resulting PBMC population was automatically counted (ViCell) and immediately used for further assays.

### Target Cells

For the assessment of MCSP-targeting bispecific antigen binding molecules, the following tumor cell lines were used: the human melanoma cell line WM266-4 (ATCC #CRL-1676), derived from a metastatic site of a malignant melanoma and expressing high levels of human MCSP; and the human melanoma cell line MV-3 (a kind gift from The Radboud University Nijmegen Medical Centre), expressing medium levels of human MCSP.

For the assessment of CEA-targeting bispecific antigen binding molecules, the following tumor cell lines were used: the human gastric cancer cell line MKN45 (DSMZ #ACC 409), expressing very high levels of human CEA; the human female Caucasian colon adenocarcinoma cell line LS-174T (ECACC #87060401), expressing medium to low levels of human CEA; the human epithelioid pancreatic carcinoma cell line Panc-1 (ATCC #CRL-1469), expressing (very) low levels of human CEA; and a murine colon carcinoma cell line MC38-huCEA, that was engineered in-house to stably express human CEA.

In addition, a human T cell leukaemia cell line, Jurkat (ATCC #TIB-152), was used to assess binding of different bispecific constructs to human CD3 on cells.

### Example 1 Preparation, Purification and Characterization of Bispecific Antigen Binding Molecules

The heavy and light chain variable region sequences were subcloned in frame with either the constant heavy chain or the constant light chain pre-inserted into the respective recipient mammalian expression vector. The antibody expression was driven by an MPSV promoter and a synthetic polyA signal sequence is located at the 3′ end of the CDS. In addition each vector contained an EBV OriP sequence.

The molecules were produced by co-transfecting HEK293 EBNA cells with the mammalian expression vectors. Exponentially growing HEK293 EBNA cells were transfected using the calcium phosphate method. Alternatively, HEK293 EBNA cells growing in suspension were transfected using polyethylenimine (PEI). For preparation of “1+1 IgG scFab, one armed / one armed inverted” constructs, cells were transfected with the corresponding expression vectors in a 1:1:1 ratio (“vector heavy chain” : “vector light chain” : “vector heavy chain-scFab”). For preparation of “2+1 IgG scFab” constructs, cells were transfected with the corresponding expression vectors in a 1:2:1 ratio (“vector heavy chain” : “vector light chain” : “vector heavy chain-scFab”). For preparation of “1+1 IgG Crossfab” constructs, cells were transfected with the corresponding expression vectors in a 1:1:1:1 ratio (“vector second heavy chain” : “vector first light chain” : “vector light chain Crossfab” : “vector first heavy chain-heavy chain Crossfab”). For preparation of “2+1 IgG Crossfab” constructs cells were transfected with the corresponding expression vectors in a 1:2:1:1 ratio (“vector second heavy chain” : “vector light chain” : “vector first heavy chain-heavy chain Crossfab)” : “vector light chain Crossfab”. For preparation of the “2+1 IgG Crossfab, linked light chain” construct, cells were transfected with the corresponding expression vectors in a 1:1:1:1 ratio (“vector heavy chain” : “vector light chain” : “vector heavy chain (CrossFab-Fab-Fc)” : “vector linked light chain”). For preparation of the “1+1 CrossMab” construct, cells were transfected with the corresponding expression vectors in a 1:1:1:1 ratio (“vector first heavy chain” : “vector second heavy chain” : “vector first light chain” : “vector second light chain”). For preparation of the “1+1 IgG Crossfab light chain fusion ” construct, cells were transfected with the corresponding expression vectors in a 1:1:1:1 ratio (“vector first heavy chain” : “vector second heavy chain” : “vector light chain Crossfab” : “vector second light chain”).

For transfection using calcium phosphate cells were grown as adherent monolayer cultures in T-flasks using DMEM culture medium supplemented with 10 % (v/v) FCS, and transfected when they were between 50 and 80% confluent. For the transfection of a T150 flask, 15 million cells were seeded 24 hours before transfection in 25 ml DMEM culture medium supplemented with FCS (at 10% v/v final), and cells were placed at 37° C. in an incubator with a 5% CO₂ atmosphere overnight. For each T150 flask to be transfected, a solution of DNA, CaCl₂ and water was prepared by mixing 94 µg total plasmid vector DNA divided in the corresponding ratio, water to a final volume of 469 µl and 469 µl of a 1 M CaCl₂ solution. To this solution, 938 µl of a 50 mM HEPES, 280 mM NaCl, 1.5 mM Na₂HPO₄ solution at pH 7.05 were added, mixed immediately for 10 s and left to stand at room temperature for 20 s. The suspension was diluted with 10 ml of DMEM supplemented with 2% (v/v) FCS, and added to the T150 in place of the existing medium. Subsequently, additional 13 ml of transfection medium were added. The cells were incubated at 37° C., 5% CO₂ for about 17 to 20 hours, then medium was replaced with 25 ml DMEM, 10 % FCS. The conditioned culture medium was harvested approximately 7 days post-media exchange by centrifugation for 15 min at 210 × g, sterile filtered (0.22 • m filter), supplemented with sodium azide to a final concentration of 0.01 % (w/v), and kept at 4° C.

For transfection using polyethylenimine (PEI) HEK293 EBNA cells were cultivated in suspension in serum free CD CHO culture medium. For the production in 500 ml shake flasks, 400 million HEK293 EBNA cells were seeded 24 hours before transfection. For transfection cells were centrifuged for 5 min at 210 × g, and supernatant was replaced by 20 ml pre-warmed CD CHO medium. Expression vectors were mixed in 20 ml CD CHO medium to a final amount of 200 µg DNA. After addition of 540 µl PEI, the mixture was vortexed for 15 s and subsequently incubated for 10 min at room temperature. Afterwards cells were mixed with the DNA/PEI solution, transferred to a 500 ml shake flask and incubated for 3 hours at 37° C. in an incubator with a 5% CO₂ atmosphere. After the incubation time 160 ml F17 medium was added and cells were cultivated for 24 hours. One day after transfection 1 mM valproic acid and 7% Feed 1 (Lonza) were added. After a cultivation of 7 days, supernatant was collected for purification by centrifugation for 15 min at 210 × g, the solution was sterile filtered (0.22 µm filter), supplemented with sodium azide to a final concentration of 0.01 % w/v, and kept at 4° C.

The secreted proteins were purified from cell culture supernatants by Protein A affinity chromatography, followed by a size exclusion chromatography step.

For affinity chromatography supernatant was loaded on a HiTrap ProteinA HP column (CV = 5 ml, GE Healthcare) equilibrated with 25 ml 20 mM sodium phosphate, 20 mM sodium citrate, pH 7.5 or 40 ml 20 mM sodium phosphate, 20 mM sodium citrate, 0.5 M sodium chloride, pH 7.5. Unbound protein was removed by washing with at least ten column volumes 20 mM sodium phosphate, 20 mM sodium citrate, 0.5 M sodium chloride pH 7.5, followed by an additional wash step using six column volumes 10 mM sodium phosphate, 20 mM sodium citrate, 0.5 M sodium chloride pH 5.45. Subsequently, the column was washed with 20 ml 10 mM MES, 100 mM sodium chloride, pH 5.0, and target protein was eluted in six column volumes 20 mM sodium citrate, 100 mM sodium chloride, 100 mM glycine, pH 3.0. Alternatively, target protein was eluted using a gradient over 20 column volumes from 20 mM sodium citrate, 0.5 M sodium chloride, pH 7.5 to 20 mM sodium citrate, 0.5 M sodium chloride, pH 2.5. The protein solution was neutralized by adding ⅒ of 0.5 M sodium phosphate, pH 8. The target protein was concentrated and filtrated prior to loading on a HiLoad Superdex 200 column (GE Healthcare) equilibrated with 25 mM potassium phosphate, 125 mM sodium chloride, 100 mM glycine solution of pH 6.7. For the purification of 1+1 IgG Crossfab the column was equilibrated with 20 mM histidine, 140 mM sodium chloride solution of pH 6.0.

The protein concentration of purified protein samples was determined by measuring the optical density (OD) at 280 nm, using the molar extinction coefficient calculated on the basis of the amino acid sequence. Purity and molecular weight of the bispecific constructs were analyzed by SDS-PAGE in the presence and absence of a reducing agent (5 mM 1,4-dithiotreitol) and staining with Coomassie (SimpleBlue™ SafeStain from Invitrogen) using the NuPAGE® Pre-Cast gel system (Invitrogen, USA) was used according to the manufacturer’s instructions (4-12% Tris-Acetate gels or 4-12% Bis-Tris). Alternatively, purity and molecular weight of molecules were analyzed by CE-SDS analyses in the presence and absence of a reducing agent, using the Caliper LabChip GXII system (Caliper Lifescience) according to the manufacturer’s instructions.

The aggregate content of the protein samples was analyzed using a Superdex 200 10/300GL analytical size-exclusion chromatography column (GE Healthcare) in 2 mM MOPS, 150 mM NaCl, 0.02% (w/v) NaN₃, pH 7.3 running buffer at 25° C. Alternatively, the aggregate content of antibody samples was analyzed using a TSKgel G3000 SW XL analytical size-exclusion column (Tosoh) in 25 mM K₂HPO₄, 125 mM NaCl, 200 mM L-arginine monohydrocloride, 0.02% (w/v) NaN₃, pH 6.7 running buffer at 25° C.

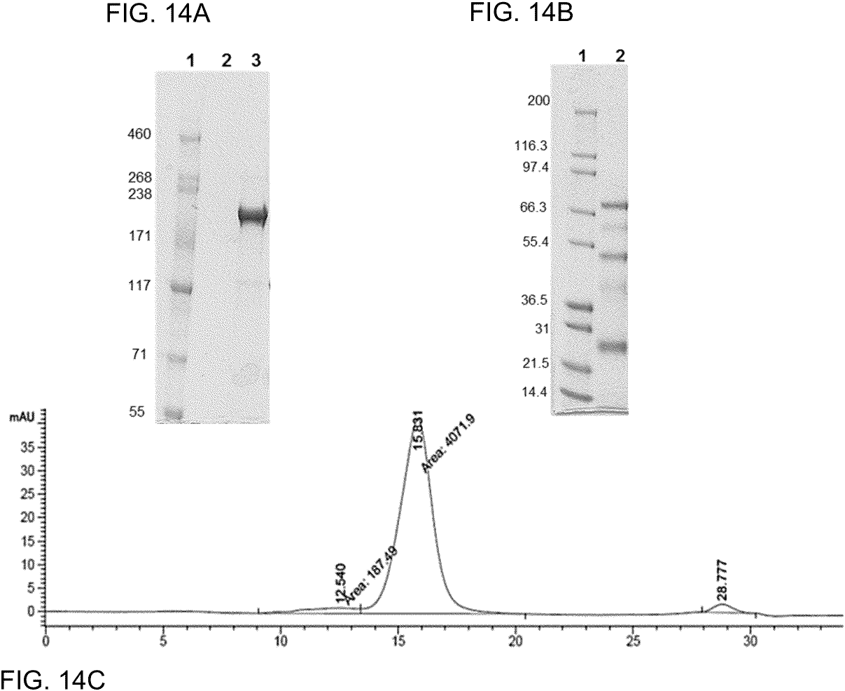

FIG. 2 - FIG. 14 show the results of the SDS PAGE and analytical size exclusion chromatography and Table 2A shows the yields, aggregate content after Protein A, and final monomer content of the preparations of the different bispecific constructs.

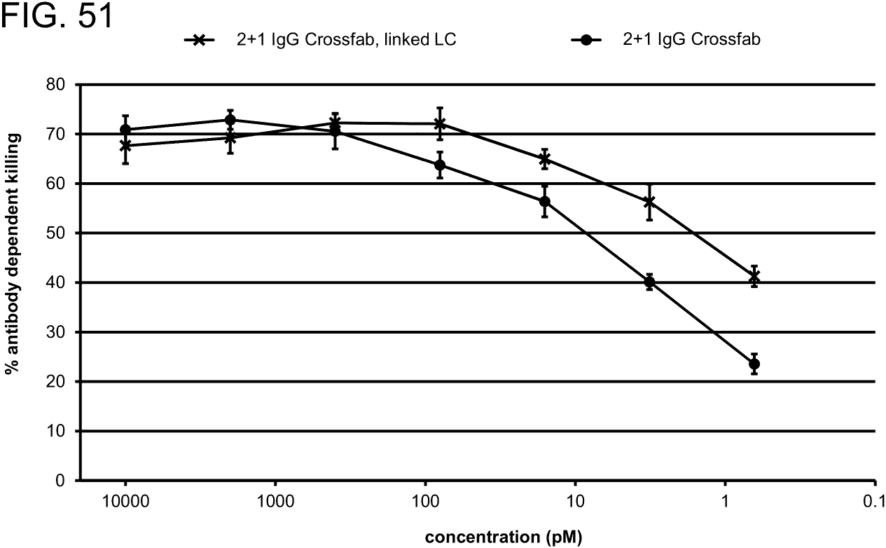

FIG. 47 shows the result of the CE-SDS analyses of the anti-CD3/anti-MCSP bispecific “2+1 IgG Crossfab, linked light chain” construct (see SEQ ID NOs 3, 5, 29 and 179). 2 µg sample was used for analyses. FIG. 48 shows the result of the analytical size exclusion chromatography of the final product (20 µg sample injected).

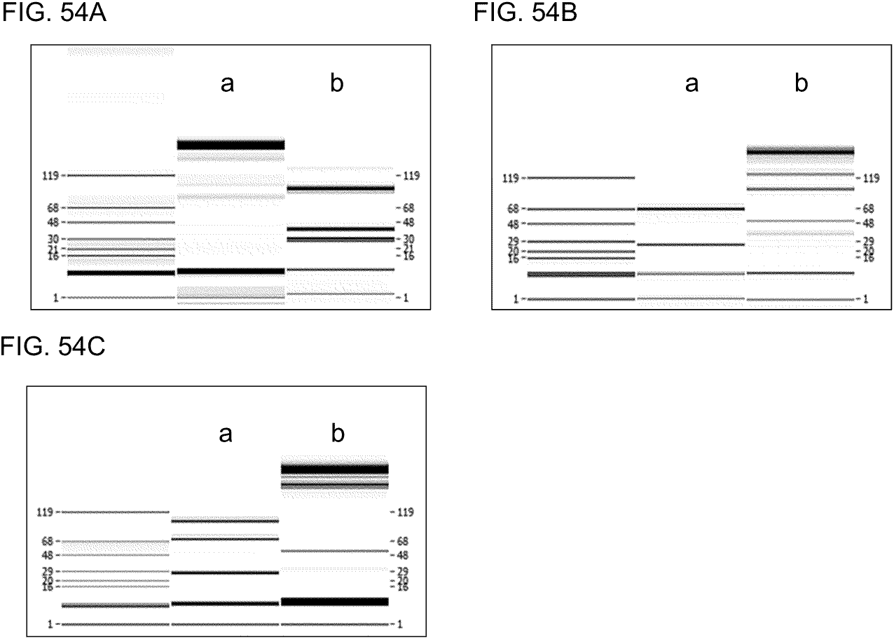

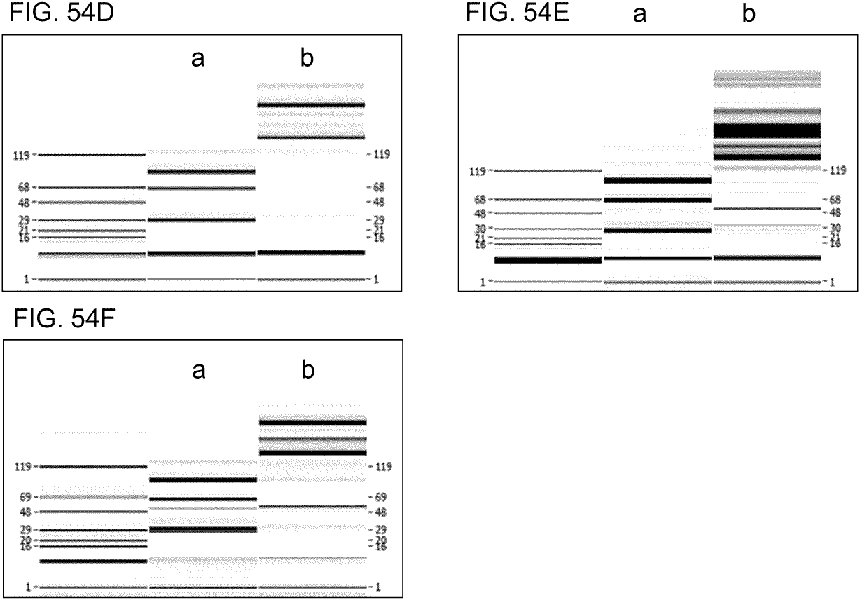

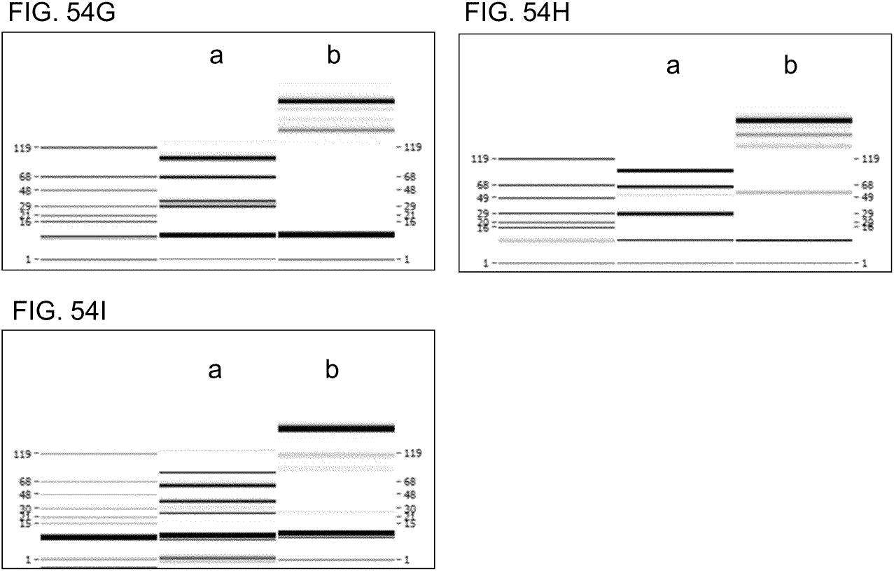

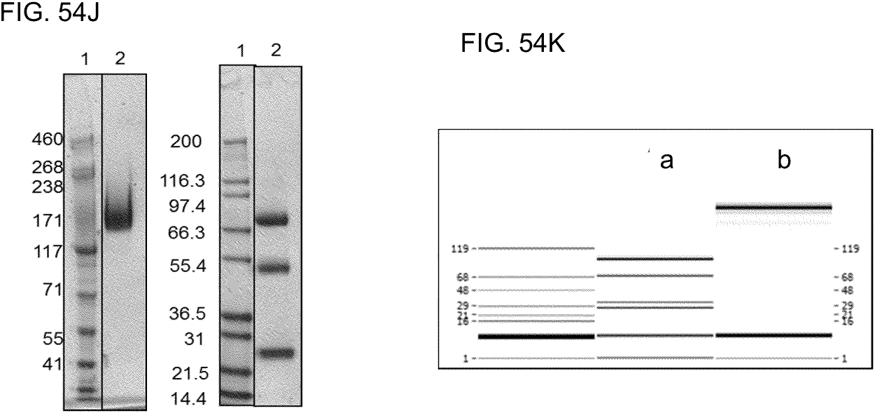

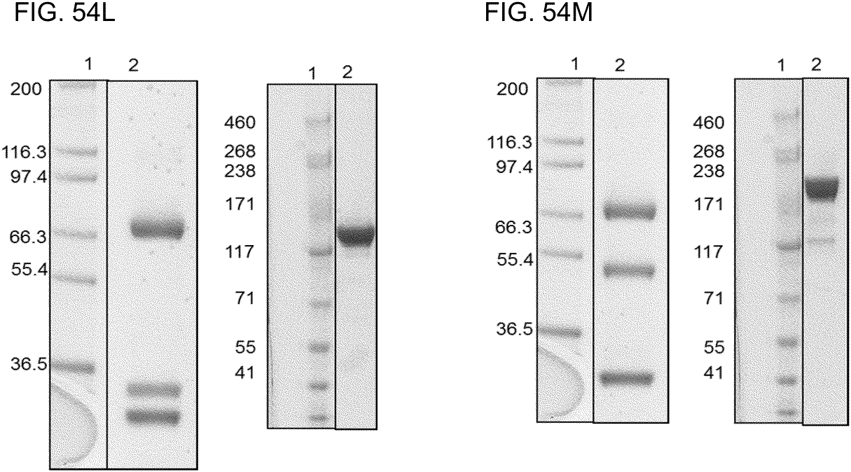

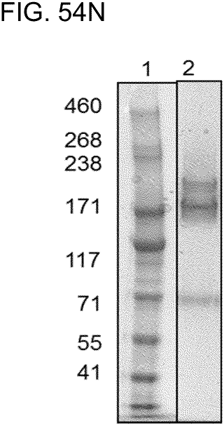

FIG. 54A - FIG. 54N show the results of the CE-SDS and SDS PAGE analyses of various constructs, and Table 2A shows the yields, aggregate content after Protein A and final monomer content of the preparations of the different bispecific constructs.

TABLE 2A

Yields, aggregate content after Protein A and final monomer content.

| Construct | Yield [mg/l] | Aggregate content after Protein A [%] | HMW [%] | LMW [%] | Monomer [%] |
|---|---|---|---|---|---|
| MCSP | | | | | |
| 2+1 IgG Crossfab; VH/VL exchange (LC007/V9) (SEQ ID NOs 3, 5, 29, 33) | 12.8 | 2.2 | 0 | 0 | 100 |
| 2+1 IgG Crossfab; VH/VL exchange (LC007/FN18) (SEQ ID NOs 3, 5, 35, 37) | 3.2 | 5.7 | 0.4 | 0 | 99.6 |
| 2+1 IgG scFab, P329G LALA (SEQ ID NOs 5, 21, 23) | 11.9 | 23 | 0.3 | 0 | 99.7 |
| 2+1 IgG scFab, LALA (SEQ ID NOs 5, 17, 19) | 9 | 23 | 0 | 0 | 100 |
| 2+1 IgG scFab, P329G LALA N297D (SEQ ID NOs 5, 25, 27) | 12.9 | 32.7 | 0 | 0 | 100 |
| 2+1 IgG scFab, wt (SEQ ID NOs 5, 13, 15) | 15.5 | 31.8 | 0 | 0 | 100 |
| 1+1 IgG scFab (SEQ ID NOs 5, 21, 213) | 7 | 24.5 | 0 | 0 | 100 |
| 1+1 IgG scFab “one armed” (SEQ ID NOs 1, 3, 5) | 7.6 | 43.7 | 2.3 | 0 | 97.7 |
| 1+1 IgG scFab “one armed inverted” (SEQ ID NOs 7, 9, 11) | 1 | 27 | 7.1 | 9.1 | 83.8 |
| 1+1 IgG Crossfab; VH/VL exchange (LC007/V9) (SEQ ID NOs 5, 29, 31, 33) | 9.8 | 0 | 0 | 0 | 100 |
| 2+1 IgG Crossfab, linked light chain; VL/VH exchange (LC007/V9) (SEQ ID NOs 3, 5, 29, 179) | 0.54 | 40 | 1.4 | 0 | 98.6 |
| 1+1 IgG Crossfab; VL/VH exchange (LC007/V9) (SEQ ID NOs 5, 29, 33, 181) | 6.61 | 8.5 | 0 | 0 | 100 |
| 1+1 CrossMab; CL/CH1 exchange (LC00/V9) (SEQ ID NOs 5, 23, 183, 185) | 6.91 | 10.5 | 1.3 | 1.7 | 97 |
| 2+1 IgG Crossfab, inverted; CL/CH1 exchange (LC007/V9) (SEQ ID NOs 5, 23, 183, 187) | 9.45 | 6.1 | 0.8 | 0 | 99.2 |
| 2+1 IgG Crossfab; VL/VH exchange (M4-3 ML2/V9) (SEQ ID NOs 33, 189, 191, 193) | 36.6 | 0 | 9.5 | 35.3 | 55.2 |
| 2+1 IgG Crossfab; CL/CH1 exchange (M4-3 ML2/V9) (SEQ ID NOs 183, 189, 193, 195) | 2.62 | 12 | 2.8 | 0 | 97.2 |
| 2+1 IgG Crossfab; CL/CH1 exchange (M4-3 ML2/H2C) (SEQ ID NOs 189, 193, 199, 201) | 29.75 | 0 | 0 | 0 | 100 |
| 2+1 IgG Crossfab; CL/CH1 exchange (LC007/anti-CD3) (SEQ ID NOs 5, 23, 215, 217) | 1.2 | 0 | 1.25 | 1.65 | 97.1 |
| 2+1 IgG Crossfab, inverted; CL/CH1 exchange (LC007/anti-CD3) (SEQ ID NOs 5, 23, 215, 219) | 7.82 | 0.5 | 0 | 0 | 100 |
| EGFR | | | | | |
| 2+1 IgG scFab (SEQ ID NOs 45, 47, 53) | 5.2 | 53 | 0 | 30 | 70 |
| 1+1 IgG scFab (SEQ ID NOs 47, 53, 213) | 3.4 | 66.6 | 0 | 1.6 | 98.4 |
| 1+1 IgG scFab “one armed” (SEQ ID NOs 43, 45, 47) | 9.05 | 60.8 | 0 | 0 | 100 |
| 1+1 IgG scFab “one armed inverted” (SEQ ID NOs 11, 49, 51) | 3.87 | 58.8 | 0 | 0 | 100 |
| FAP | | | | | |
| 2+1 IgG scFab (SEQ ID NOs 57, 59, 61) | 12.57 | 53 | 0 | 0 | 100 |
| 1+1 IgG scFab | 17.95 | 41 | 0.4 | 0 | 99.6 |
| (SEQ ID NOs 57, 61, 213) | | | | | |
| 1+1 IgG scFab “one armed inverted” (SEQ ID NOs 11, 51, 55) | 2.44 | 69 | 0.6 | 0 | 99.4 |
| CEA | | | | | |
| 2+1 IgG Crossfab, inverted; VL/VH exchange (CH1A1A/V9) (SEQ ID NOs 33, 63, 65, 67) | 0.34 | 13 | 4.4 | 0 | 95.6 |
| 2+1 IgG Crossfab, inverted; CL/CH1 exchange (CH1A1A/V9) (SEQ ID NOs 65, 67, 183, 197) | 12.7 | 43 | 0 | 0 | 100 |
| 2+1 IgG Crossfab, inverted; CL/CH1 exchange (431/26/V9) (SEQ ID NOs 183, 203, 205, 207) | 7.1 | 20 | 0 | 0 | 100 |
| 1+1 IgG-Crossfab light chain fusion (CH1A1A/V9) (SEQ ID NOs 183, 209, 211, 213) | 7.85 | 27 | 4.3 | 3.2 | 92.5 |

As controls, bispecific antigen binding molecules were generated in the prior art tandem scFv format (“(scFv)₂”) and by fusing a tandem scFv to an Fc domain (“(scFv)₂-Fc”). The molecules were produced in HEK293-EBNA cells and purified by Protein A affinity chromatography followed by a size exclusion chromatographic step in an analogous manner as described above for the bispecific antigen binding molecules of the invention. Due to high aggregate formation, some of the samples had to be further purified by applying eluted and concentrated samples from the HiLoad Superdex 200 column (GE Healthcare) to a Superdex 10/300 GL column (GE Healthcare) equilibrated with 20 mM histidine, 140 mM sodium chloride, pH 6.7 in order to obtain protein with high monomer content. Subsequently, protein concentration, purity and molecular weight, and aggregate content were determined as described above.

Yields, aggregate content after the first purification step, and final monomer content for the control molecules is shown in Table 2B. Comparison of the aggregate content after the first purification step (Protein A) indicates the superior stability of the IgG Crossfab and IgG scFab constructs compared to the “(scFv)₂-Fc” and the disulfide bridge-stabilized “(dsscFv)₂-Fc” molecules.

TABLE 2B

Yields, aggregate content after Protein A and final monomer content.

| Construct | Yield [mg/l] | Aggregates after ProteinA [%] | Final | | |
|---|---|---|---|---|---|
| | | | HMW [%] | LMW [%] | Monomer [%] |
| (scFv)₂-Fc (antiMCSP/anti huCD3) | 76.5 | 40 | 0.5 | 0 | 99.5 |
| (dsscFv)₂-Fc (antiMCSP/anti huCD3) | 2.65 | 48 | 7.3 | 8.0 | 84.7 |

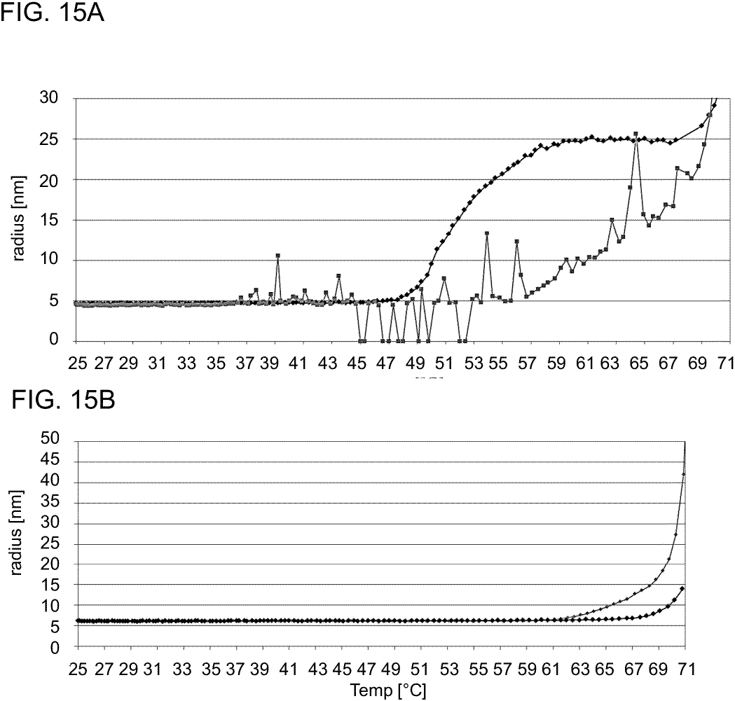

Thermal stability of the proteins was monitored by Dynamic Light Scattering (DLS). 30 • g of filtered protein sample with a protein concentration of 1 mg/ml was applied in duplicate to a Dynapro plate reader (Wyatt Technology Corporation; USA). The temperature was ramped from 25 to 75° C. at 0.05° C./min, with the radius and total scattering intensity being collected. The results are shown in FIG. 15A, FIG. 15B, and Table 2C. For the “(scFv)2-Fc” (antiMCSP/anti huCD3) molecule two aggregation points were observed, at 49° C. and 68° C. The “(dsscFv)₂-Fc” construct has an increased aggregation temperature (57° C.) as a result of the introduced disulfide bridge (FIG. 15A, Table 2C). Both, the “2+1 IgG scFab” and the “2+1 IgG Crossfab” constructs are aggregating at temperatures higher than 60° C., demonstrating their superior thermal stability as compared to the “(scFv)₂-Fc” and “(dsscFv)₂-Fc” formats (FIG. 15B, Table 2C).

TABLE 2C

Thermal stability determined by dynamic light scattering.

| Construct | T_{agg} [°C] |
|---|---|
| 2+1 IgG scFab (LC007/V9) | 68 |
| 2+1 IgG Crossfab (LC007/V9) | 65 |
| Fc-(scFv)2 (LC007/V9) | 49/68 |
| Fc-(dsscFv)2 (LC007/V9) | 57 |

### Example 2 Surface Plasmon Resonance Analysis of Fc Receptor and Target Antigen Binding

### Method

All surface plasmon resonance (SPR) experiments are performed on a Biacore T100 at 25° C. with HBS-EP as running buffer (0.01 M HEPES pH 7.4, 0.15 M NaCl, 3 mM EDTA, 0.005% Surfactant P20, Biacore, Freiburg/Germany).

### Analysis of FcR Binding of Different Fc-Variants

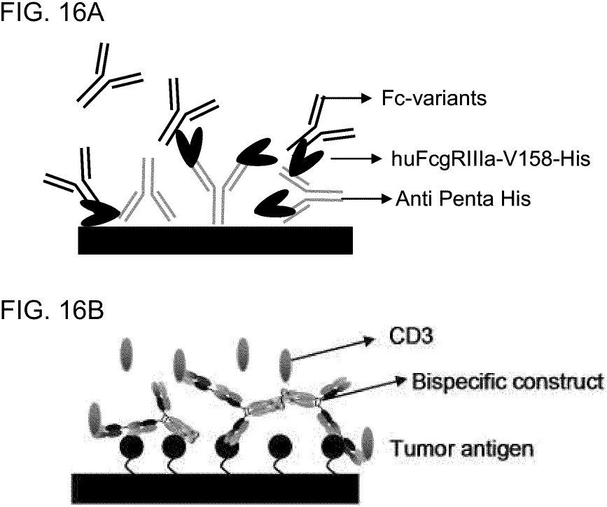

The assay setup is shown in FIG. 16A. For analyzing interaction of different Fc-variants with human FcγRIIIa-V158 and murine FcγRIV direct coupling of around 6,500 resonance units (RU) of the anti-Penta His antibody (Qiagen) is performed on a CM5 chip at pH 5.0 using the standard amine coupling kit (Biacore, Freiburg/Germany). HuFcγRIIIa-V158-K6H6 and muFcγRIV-aviHis-biotin are captured for 60 s at 4 and 10 nM respectively.

Constructs with different Fc-mutations are passed through the flow cells for 120 s at a concentration of 1000 nM with a flow rate of 30 µl/min. The dissociation is monitored for 220 s. Bulk refractive index differences are corrected for by subtracting the response obtained in a reference flow cell. Here, the Fc-variants are flown over a surface with immobilized anti-Penta His antibody but on which HBS-EP has been injected rather than HuFcγRIIIa-V158-K6H6 or muFcγRIV-aviHis-biotin. Affinity for human FcγRIIIa-V158 and murine FcγRIV was determined for wild-type Fc using a concentration range from 500 - 4000 nM.

The steady state response was used to derive the dissociation constant K_{D} by non-linear curve fitting of the Langmuir binding isotherm. Kinetic constants were derived using the Biacore T100 Evaluation Software (vAA, Biacore AB, Uppsala/Sweden), to fit rate equations for 1:1 Langmuir binding by numerical integration.

### Result

The interaction of Fc variants with human FcγRIIIa and murine FcγRIV was monitored by surface plasmon resonance. Binding to captured huFcγRIIIa-V158-K6H6 and muFcγRIV-aviHis-biotin is significantly reduced for all analyzed Fc mutants as compared to the construct with a wild-type (wt) Fc domain.

The Fc mutants with the lowest binding to the human Fcy-receptor were P329G L234A L235A (LALA) and P329G LALA N297D. The LALA mutation alone was not enough to abrogate binding to huFcγRIIIa-V158-K6H6. The Fc variant carrying only the LALA mutation had a residual binding affinity to human FcγRIIIa of 2.100 nM, while the wt Fc bound the human FcγRIIIa receptor with an affinity of 600 nM (Table 3). Both K_{D} values were derived by 1:1 binding model, using a single concentration.

Affinity to human FcγRIIIa-V158 and murine FcγRIV could only be analyzed for wt Fc. K_{D} values are listed in Table 3. Binding to the murine FcγRIV was almost completely eliminated for all analyzed Fc mutants.

TABLE 3

Affinity of Fc-variants to the human FcγRIIIa-V158 and murine FcγRIV.

| K_{D} in nM T = 25° C. | human FcγRIIIa-V158 | | murine FcγRIV | |
|---|---|---|---|---|
| | kinetic | steady state | kinetic | steady state |
| Fc-wt (SEQ ID NOs 5, 13, 15) | 600^{∗}(1200) | 3470 | 576 | 1500 |
| Fc-LALA (SEQ ID NOs 5, 17, 19) | 2130* | n.d. | n.d. | |
| Fc-P329G LALA (SEQ ID NOs 5, 21, 23) | n.d. | | n.d. | |
| Fc-P329G LALA N297D (SEQ ID NOs 5, 25, 27) | n.d. | | n.d. | |

^{∗}determined using one concentration (1000 nM)

### Analysis of Simultaneous Binding to Tumor Antigen and CD3

Analysis of simultaneous binding of the T-cell bispecific constructs to the tumor antigen and the human CD3ε was performed by direct coupling of 1650 resonance units (RU) of biotinylated D3 domain of MCSP on a sensor chip SA using the standard coupling procedure. Human EGFR was immobilized using standard amino coupling procedure. 8000 RU were immobilized on a CM5 sensor chip at pH 5.5. The assay setup is shown in FIG. 16B.

Different T-cell bispecific constructs were captured for 60 s at 200 nM. Human CD3γ(G₄S)₅CD3ε-AcTev-Fc(knob)-Avi/Fc(hole) was subsequently passed at a concentration of 2000 nM and a flow rate of 40 µl/min for 60 s. Bulk refractive index differences were corrected for by subtracting the response obtained on a reference flow cell where the recombinant CD3ε was flown over a surface with immobilized D3 domain of MCSP or EGFR without captured T-cell bispecific constructs.

### Result

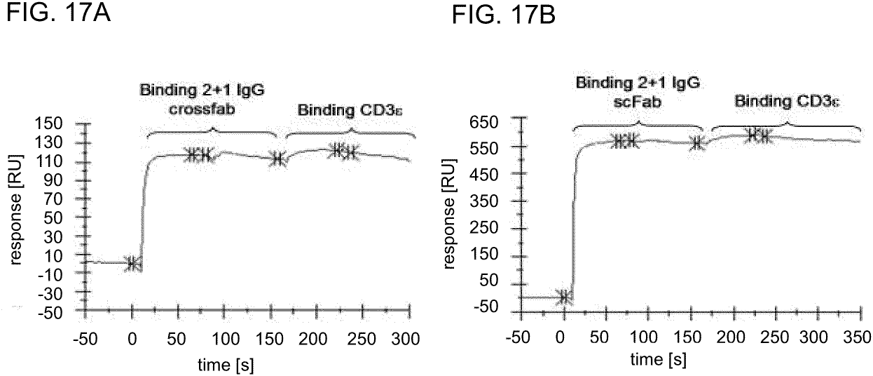

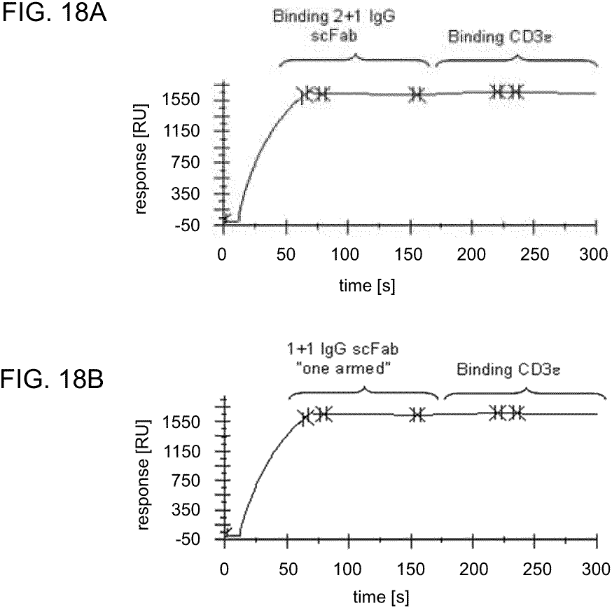

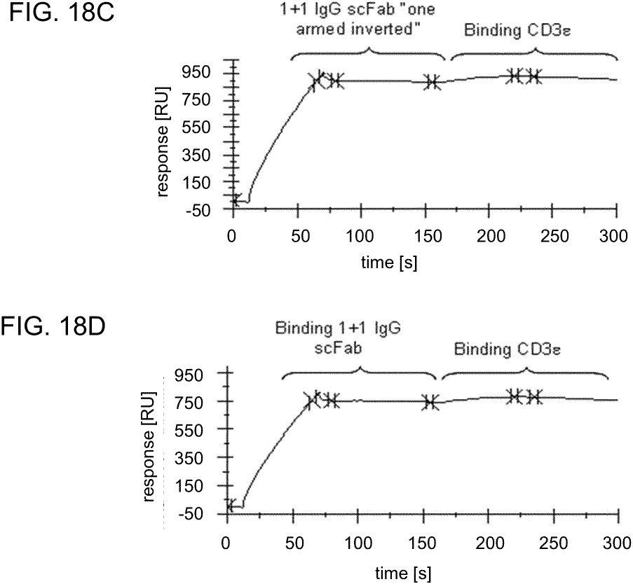

Simultaneous binding to both tumor antigen and human CD3ε was analyzed by surface plasmon resonance (FIG. 17A, FIG. 17B, and FIG. 18A - FIG. 18D). All constructs were able to bind the tumor antigen and the CD3 simultaneously. For most of the constructs the binding level (RU) after injection of human CD3ε was higher than the binding level achieved after injection of the construct alone reflecting that both tumor antigen and the human CD3ε were bound to the construct.

### Example 3 Binding of Bispecific Constructs to the Respective Target Antigen on Cells

Binding of the different bispecific constructs to CD3 on Jurkat cells (ATCC #TIB-152), and the respective tumor antigen on target cells, was determined by FACS. Briefly, cells were harvested, counted and checked for viability. 0.15 - 0.2 million cells per well (in PBS containing 0.1% BSA; 90 µl) were plated in a round-bottom 96-well plate and incubated with the indicated concentration of the bispecific constructs and corresponding IgG controls (10 µl) for 30 min at 4° C. For a better comparison, all constructs and IgG controls were normalized to same molarity. After the incubation, cells were centrifuged (5 min, 350 × g), washed with 150 µl PBS containing 0.1% BSA, resuspended and incubated for further 30 min at 4° C. with 12 µl/well of a FITC-or PE-conjugated secondary antibody. Bound constructs were detected using a FACSCantoII (Software FACS Diva). The “(scFv)₂” molecule was detected using a FITC-conjugated anti-His antibody (Lucerna, #RHIS-45F-Z). For all other molecules, a FITC- or PE-conjugated AffiniPure F(ab′)2 Fragment goat anti-human IgG Fcγ Fragment Specific (Jackson Immuno Research Lab # 109-096-098 / working solution 1:20, or #109-116-170 / working solution 1:80, respectively) was used. Cells were washed by addition of 120 µl/well PBS containing 0.1% BSA and centrifugation at 350 × g for 5 min. A second washing step was performed with 150 µl/well PBS containing 0.1% BSA. Unless otherwise indicated, cells were fixed with 100 µl/well fixation buffer (BD #554655) for 15 min at 4° C. in the dark, centrifuged for 6 min at 400 × g and kept in 200 µl/well PBS containing 0.1% BSA until the samples were measured with FACS CantoII. EC50 values were calculated using the GraphPad Prism software.

In a first experiment, different bispecific constructs targeting human MCSP and human CD3 were analyzed by flow cytometry for binding to human CD3 expressed on Jurkat, human T cell leukaemia cells, or to human MCSP on Colo-38 human melanoma cells.

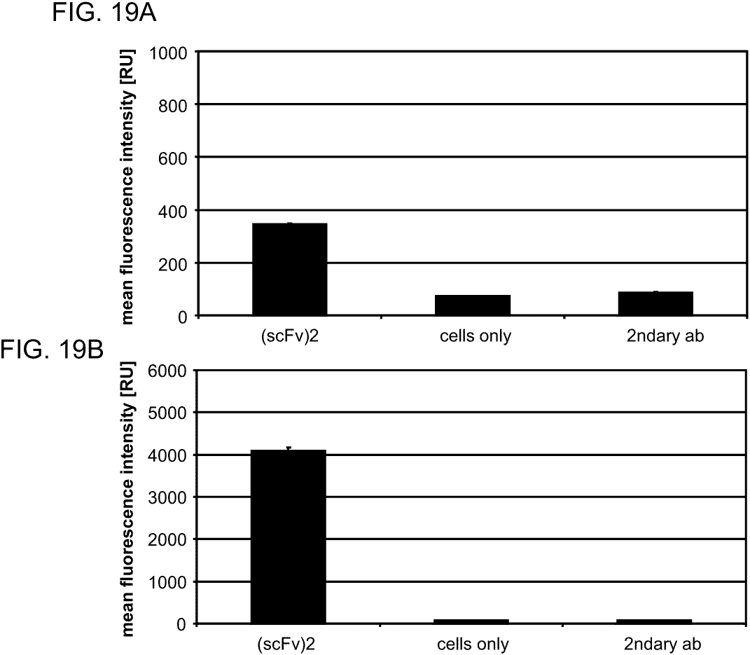

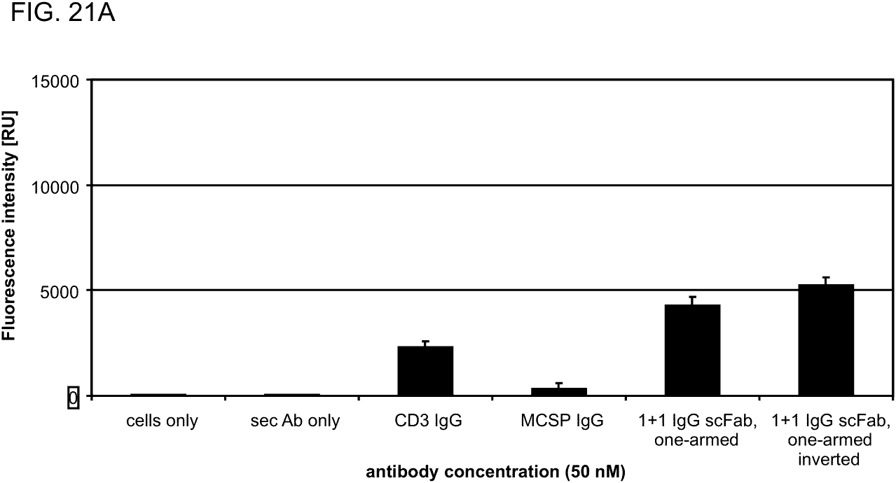

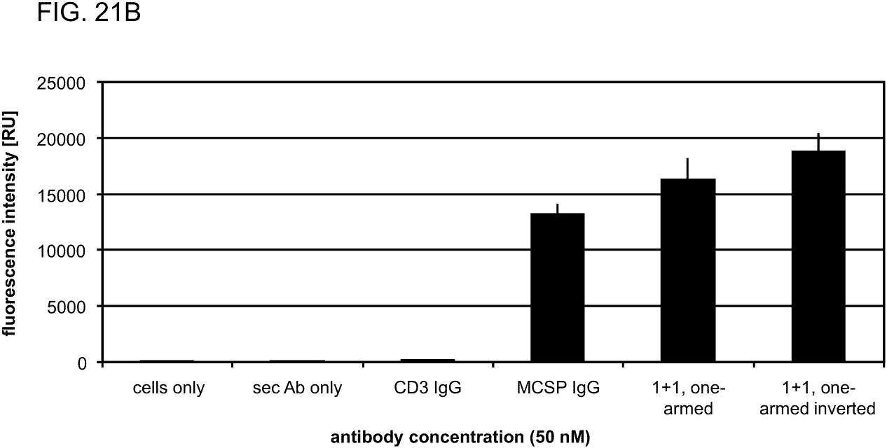

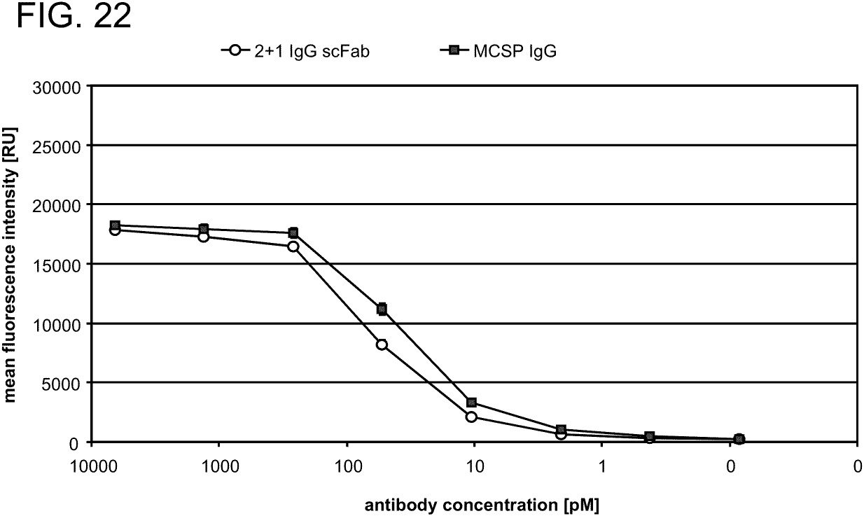

Results are presented in FIG. 19 - FIG. 21, which show the mean fluorescence intensity of cells that were incubated with the bispecific molecule, control IgG, the secondary antibody only, or left untreated.

As shown in FIG. 19A and FIG. 19B, for both antigen binding moieties of the “(scFv)₂” molecule, i.e. CD3 (FIG. 19A) and MCSP (FIG. 19B), a clear binding signal is observed compared to the control samples.

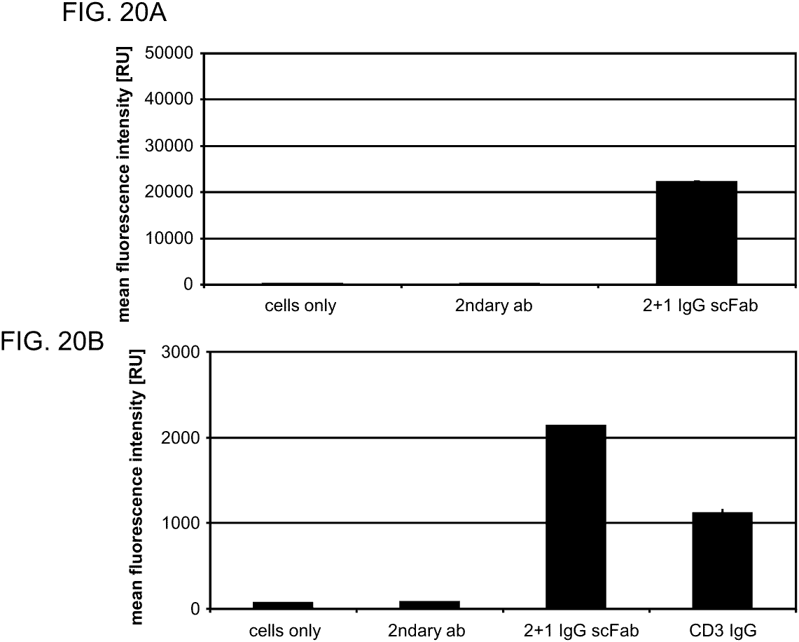

The “2+1 IgG scFab” molecule (SEQ ID NOs 5, 17, 19) shows good binding to huMCSP on Colo-38 cells (FIG. 20A). The CD3 moiety binds CD3 slightly better than the reference anti-human CD3 IgG (FIG. 20B).

As depicted in FIG. 21A, the two “1+1” constructs show comparable binding signals to human CD3 on cells. The reference anti-human CD3 IgG gives a slightly weaker signal. In addition, both constructs tested (“1+1 IgG scFab, one-armed” (SEQ ID NOs 1, 3, 5) and “1+1 IgG scFab, one-armed inverted” (SEQ ID NOs 7, 9, 11)) show comparable binding to human MCSP on cells (FIG. 21B). The binding signal obtained with the reference anti-human MCSP IgG is slightly weaker.

In another experiment, the purified “2+1 IgG scFab” bispecific construct (SEQ ID NOs 5, 17, 19) and the corresponding anti human MCSP IgG were analyzed by flow cytometry for dose-dependent binding to human MCSP on Colo-38 human melanoma cells, to determine whether the bispecific construct binds to MCSP via one or both of its “arms”. As depicted in FIG. 22, the “2+1 IgG scFab” construct shows the same binding pattern as the MCSP IgG.

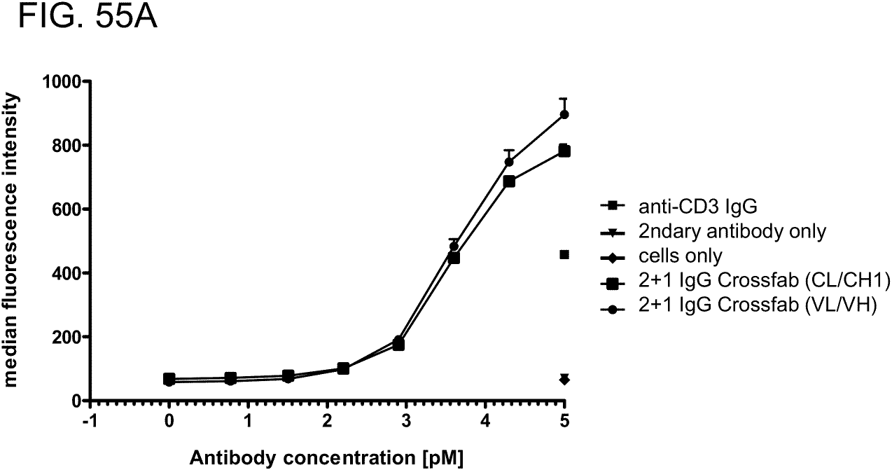

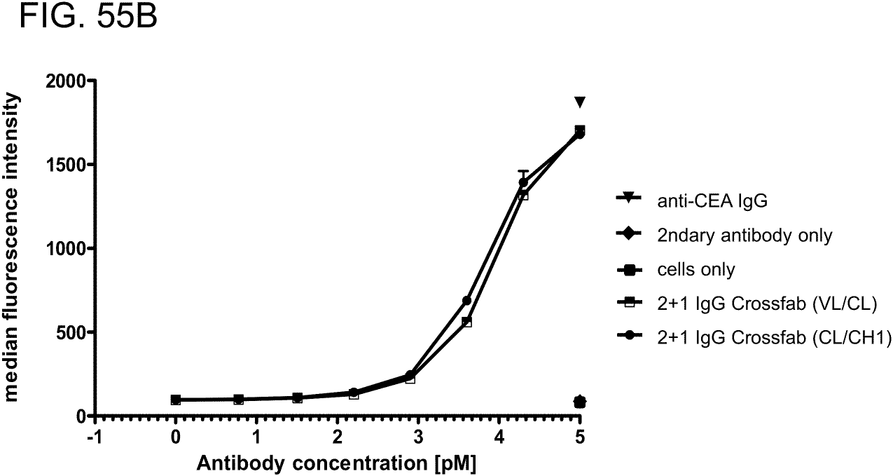

In yet another experiment, the binding of CD3/CEA “2+1 IgG Crossfab, inverted” bispecific constructs with either a VL/VH (see SEQ ID NOs 33, 63, 65, 67) or a CL/CH1 exchange (see SEQ ID NOs 66, 67, 183, 197) in the Crossfab fragment to human CD3, expressed by Jurkat cells, or to human CEA, expressed by LS-174T cells, was assessed. As a control, the equivalent maximum concentration of the corresponding IgGs and the background staining due to the labeled 2ndary antibody (goat anti-human FITC-conjugated AffiniPure F(ab′)2 Fragment, Fcγ Fragment-specific, Jackson Immuno Research Lab # 109-096-098) were assessed as well. As illustrated in FIG. 55A and FIG. 55B, both constructs show good binding to human CEA, as well as to human CD3 on cells. The calculated EC50 values were 4.6 and 3.9 nM (CD3), and 9.3 and 6.7 nM (CEA) for the “2+1 IgG Crossfab, inverted (VL/VH)” and the “2+1 IgG Crossfab, inverted (CL/CH1)” constructs, respectively.

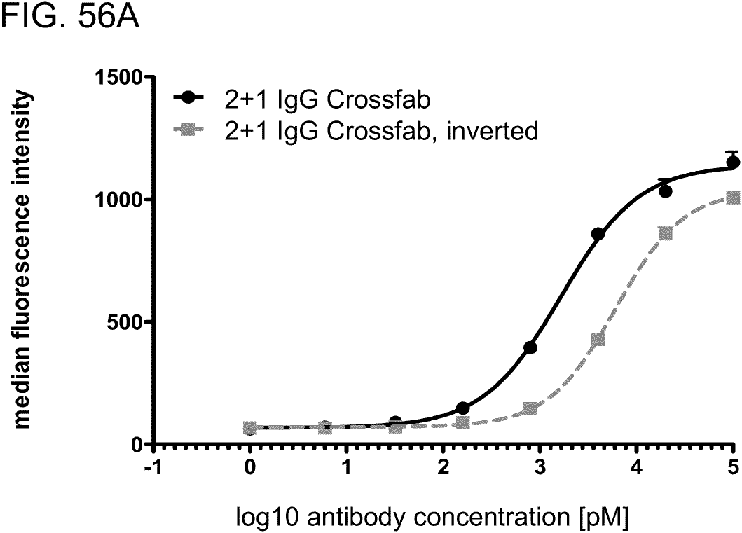

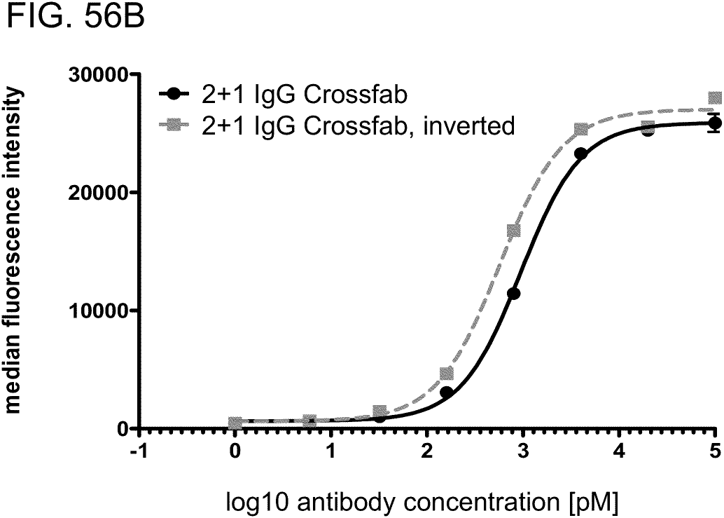

In another experiment, the binding of CD3/MCSP “2+1 IgG Crossfab” (see SEQ ID NOs 3, 5, 29, 33) and “2+1 IgG Crossfab, inverted” (see SEQ ID NOs 5, 23, 183, 187) constructs to human CD3, expressed by Jurkat cells, or to human MCSP, expressed by WM266-4 cells, was assessed. FIG. 56A and FIG. 56B show that, while binding of both constructs to MCSP on cells was comparably good, the binding of the “inverted” construct to CD3 was reduced compared to the other construct.

The calculated EC50 values were 6.1 and 1.66 nM (CD3), and 0.57 and 0.95 nM (MCSP) for the “2+1 IgG Crossfab, inverted” and the “2+1 IgG Crossfab” constructs, respectively.

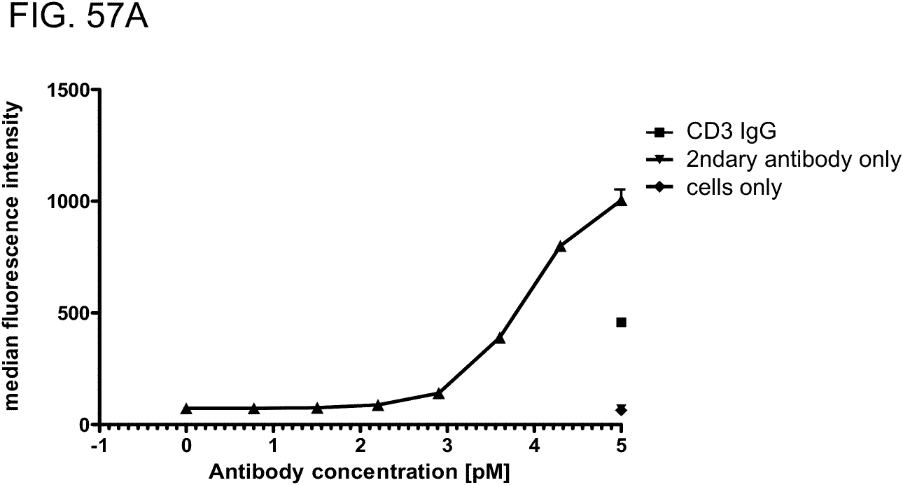

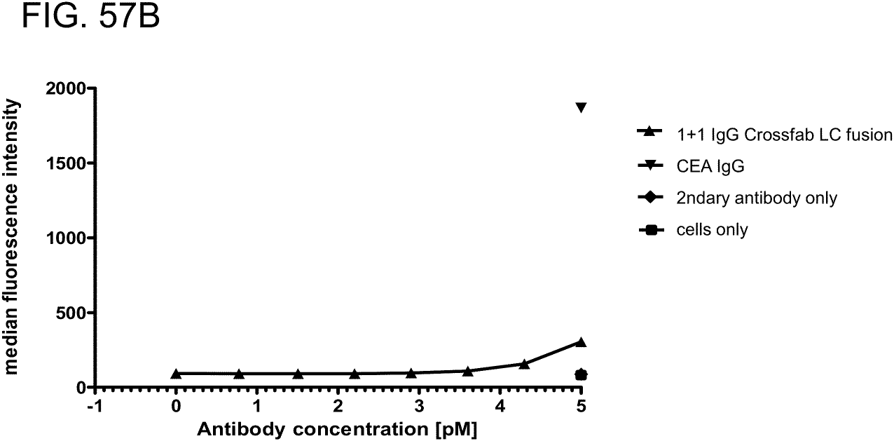

In a further experiment, binding of the “1+1 IgG Crossfab light chain (LC) fusion” construct (SEQ ID NOs 183, 209, 211, 213) to human CD3, expressed by Jurkat cells, and to human CEA, expressed by LS-174T cells was determined. As a control, the equivalent maximum concentration of the corresponding anti-CD3 and anti-CEA IgGs and the background staining due to the labeled 2ndary antibody (goat anti-human FITC-conjugated AffiniPure F(ab′)2 Fragment, Fcγ Fragment-specific, Jackson Immuno Research Lab #109-096-098) were assessed as well. As depicted in FIG. 57A and FIG. 57B, the binding of the “1+1 IgG Crossfab LC fusion” to CEA appears to be greatly reduced, whereas the binding to CD3 was at least comparable to the reference IgG.

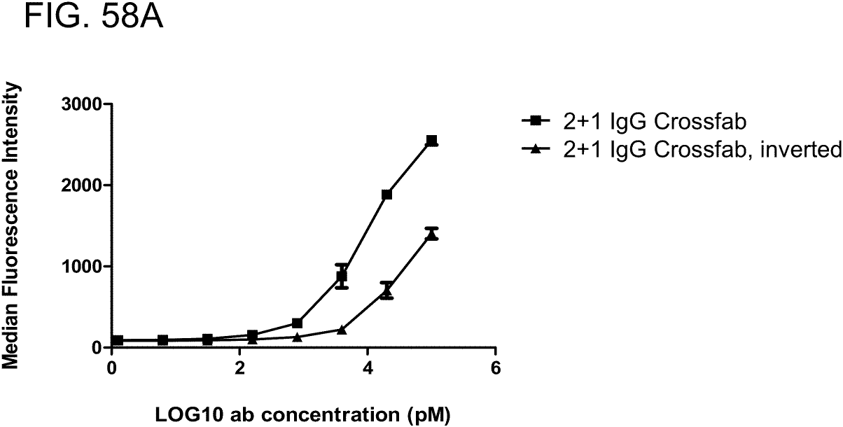

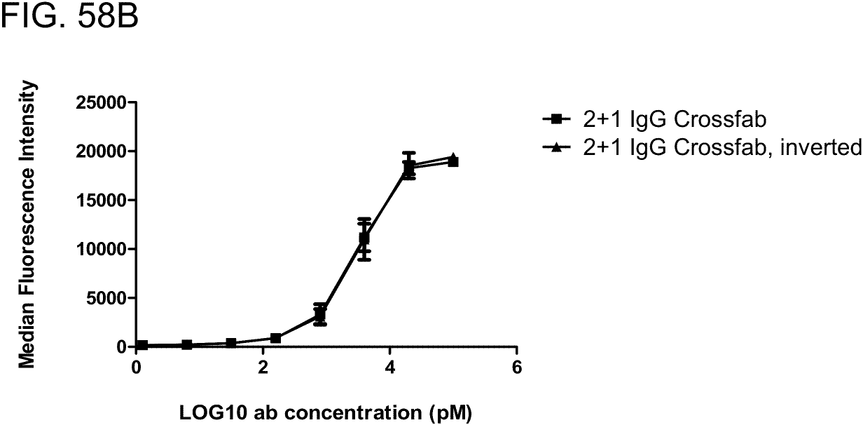

In a final experiment, binding of the “2+1 IgG Crossfab” (SEQ ID NOs 5, 23, 215, 217) and the “2+1 IgG Crossfab, inverted” (SEQ ID NOs 5, 23, 215, 219) constructs to human CD3, expressed by Jurkat cells, and to human MCSP, expressed by WM266-4 tumor cells was determined. As depicted in FIG. 58A and FIG. 58B the binding to human CD3 was reduced for the “2+1 IgG Crossfab, inverted” compared to the other construct, but the binding to human MCSP was comparably good. The calculated EC50 values were 10.3 and 32.0 nM (CD3), and 3.1 and 3.4 nM (MCSP) for the “2+1 IgG Crossfab” and the “2+1 IgG Crossfab, inverted” construct, respectively.

### Example 4 FACS Analysis of Surface Activation Markers on Primary Human T Cells Upon Engagement of Bispecific Constructs

The purified huMCSP-huCD3-targeting bispecific “2+1 IgG scFab” (SEQ ID NOs 5, 17, 19) and “(scFv)₂” molecules were tested by flow cytometry for their potential to up-regulate the early surface activation marker CD69, or the late activation marker CD25 on CD8⁺ T cells in the presence of human MCSP-expressing tumor cells.

Briefly, MCSP-positive Colo-38 cells were harvested with Cell Dissociation buffer, counted and checked for viability. Cells were adjusted to 0.3 × 10⁶ (viable) cells per ml in AIM-V medium, 100 µl of this cell suspension per well were pipetted into a round-bottom 96-well plate (as indicated). 50 µl of the (diluted) bispecific construct were added to the cell-containing wells to obtain a final concentration of 1 nM. Human PBMC effector cells were isolated from fresh blood of a healthy donor and adjusted to 6 × 10⁶ (viable) cells per ml in AIM-V medium. 50 µl of this cell suspension was added per well of the assay plate (see above) to obtain a final E:T ratio of 10:1. To analyze whether the bispecific constructs are able to activate T cells exclusively in the presence of target cells expressing the tumor antigen huMCSP, wells were included that contained 1 nM of the respective bispecific molecules, as well as PBMCs, but no target cells.

After incubation for 15 h (CD69), or 24 h (CD25) at 37° C., 5% CO₂, cells were centrifuged (5 min, 350 × g) and washed twice with 150 µl/well PBS containing 0.1% BSA. Surface staining for CD8 (mouse IgG1,κ; clone HIT8a; BD #555635), CD69 (mouse IgG1; clone L78; BD #340560) and CD25 (mouse IgG1,κ; clone M-A251; BD #555434) was performed at 4° C. for 30 min, according to the supplier’s suggestions. Cells were washed twice with 150 µl/well PBS containing 0.1% BSA and fixed for 15 min at 4° C., using 100 µl/well fixation buffer (BD #554655). After centrifugation, the samples were resuspended in 200 µl/well PBS with 0.1% BSA and analyzed using a FACS CantoII machine (Software FACS Diva).

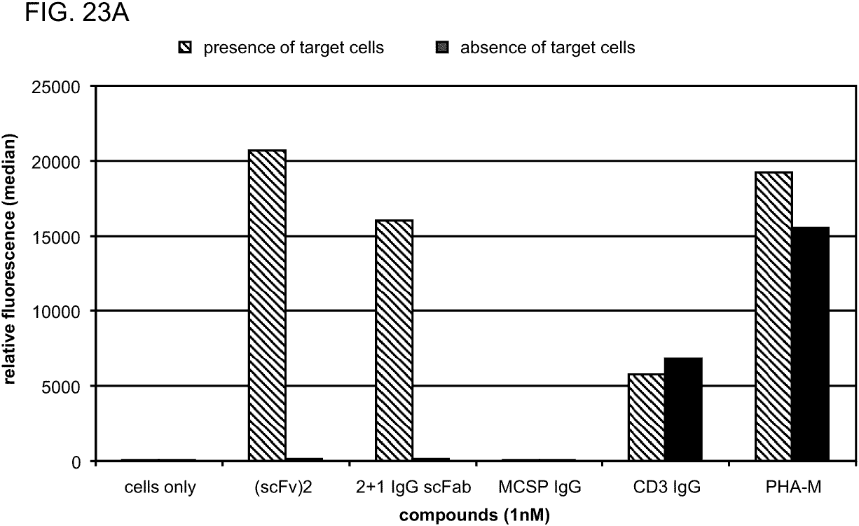

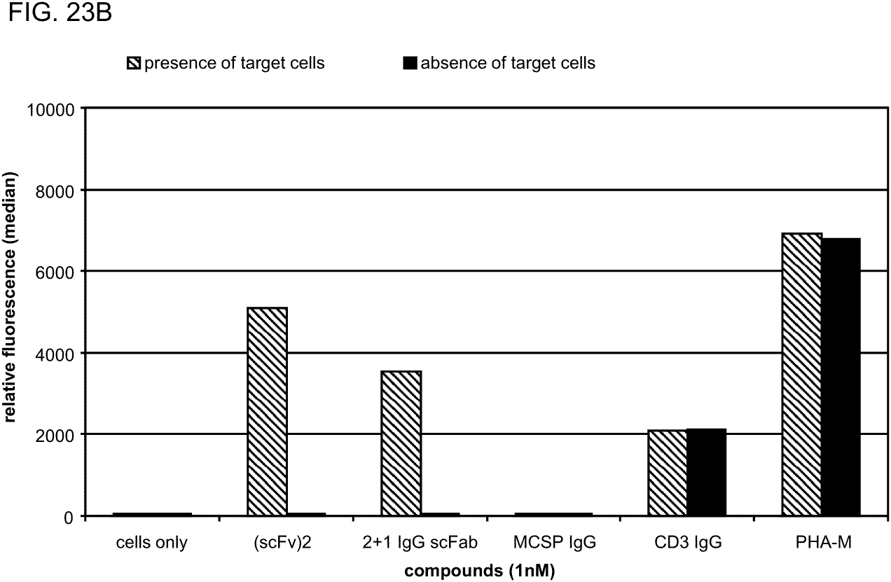

FIG. 23A and FIG. 23B depict the expression level of the early activation marker CD69 (FIG. 23A), or the late activation marker CD25 (FIG. 23B) on CD8⁺ T cells after 15 hours or 24 hours incubation, respectively. Both constructs induce up-regulation of both activation markers exclusively in the presence of target cells. The “(scFv)₂” molecule seems to be slightly more active in this assay than the “2+1 IgG scFab” construct.

The purified huMCSP-huCD3-targeting bispecific “2+1 IgG scFab” and “(scFv)₂” molecules were further tested by flow cytometry for their potential to up-regulate the late activation marker CD25 on CD8⁺ T cells or CD4⁺ T cells in the presence of human MCSP-expressing tumor cells. Experimental procedures were as described above, using human pan T effector cells at an E:T ratio of 5:1 and an incubation time of five days.

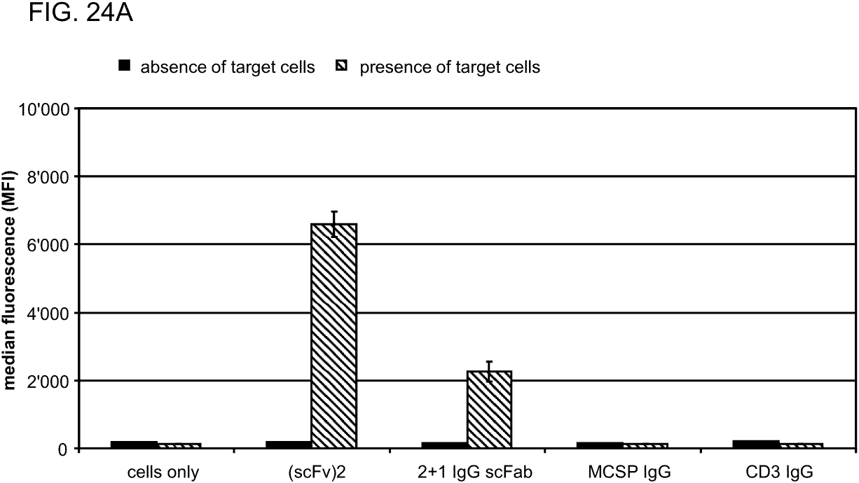

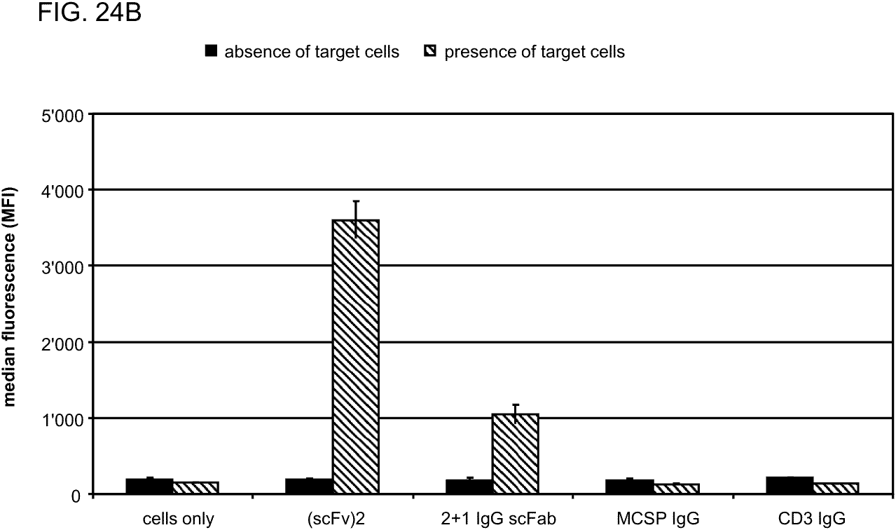

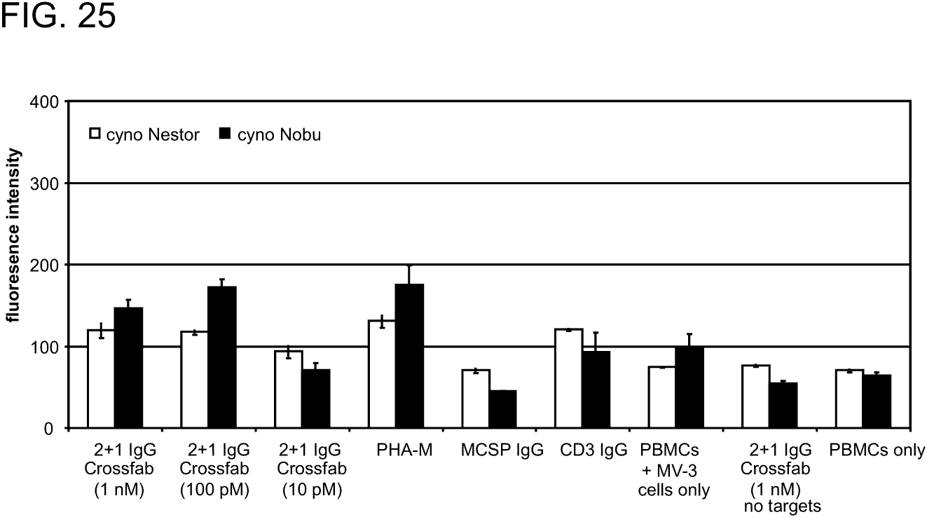

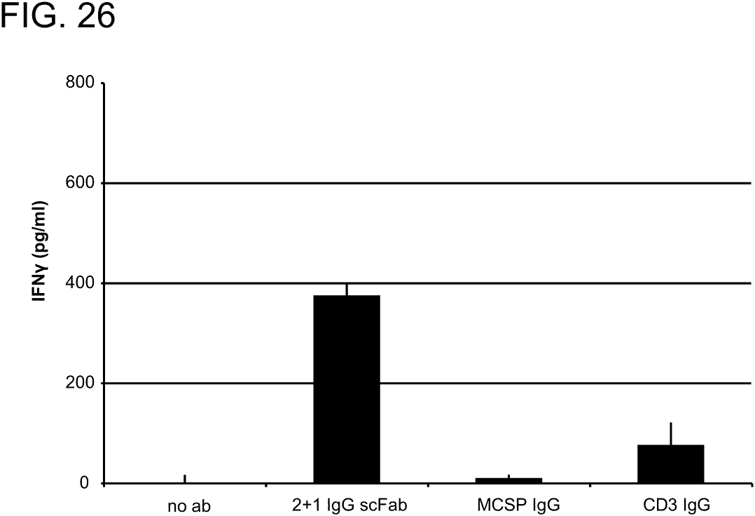

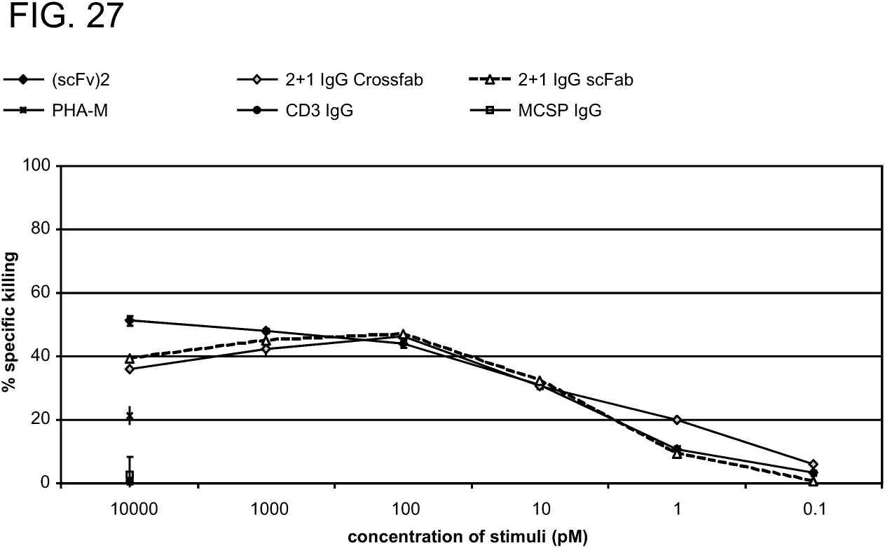

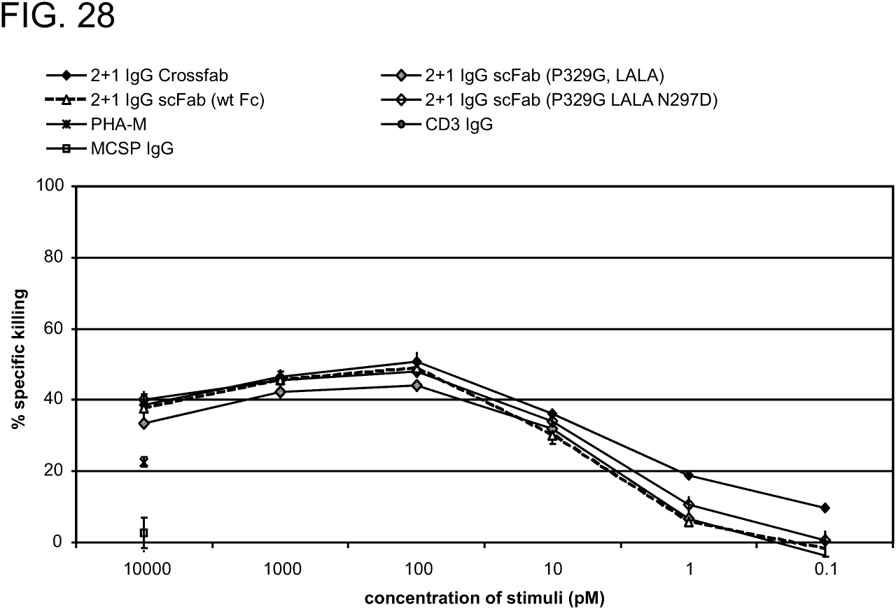

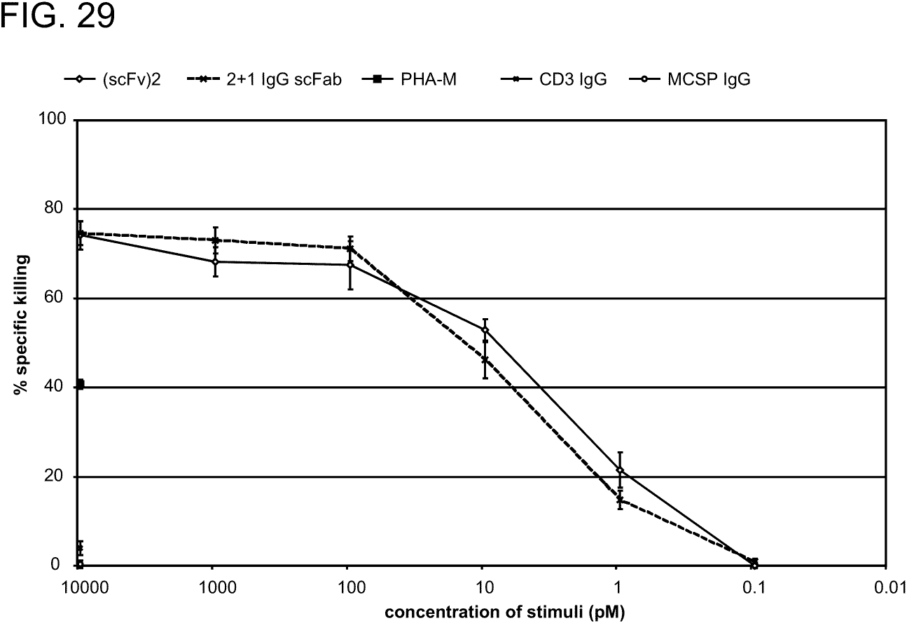

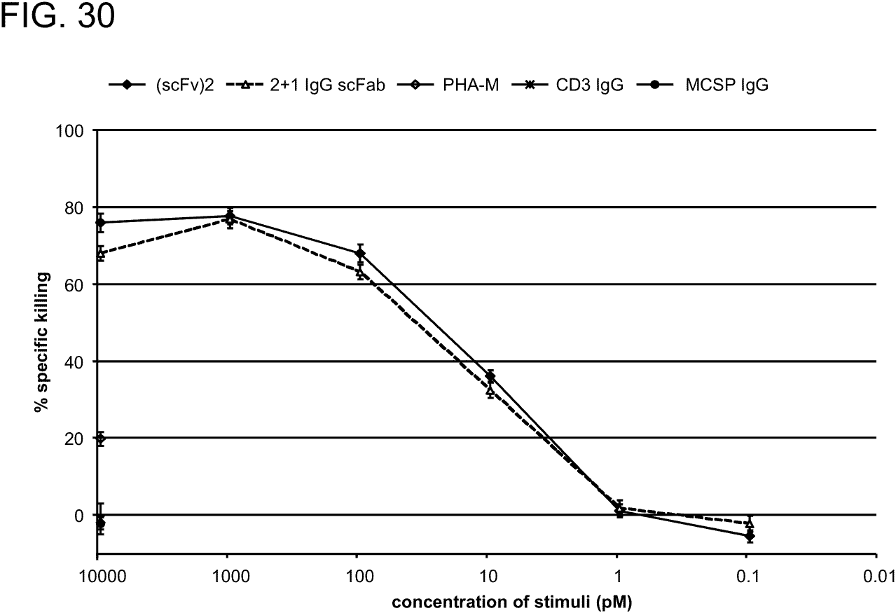

FIG. 24A and FIG. 24B showthat both constructs induce up-regulation of CD25 exclusively in the presence of target cells on both, CD8⁺ (FIG. 24A) as well as CD4⁺ (FIG. 24B) T cells. The “2+1 IgG scFab” construct seems to induce less up-regulation of CD25 in this assay, compared to the “(scFv)₂” molecule. In general, the up-regulation of CD25 is more pronounced on CD8⁺ than on CD4⁺ T cells.

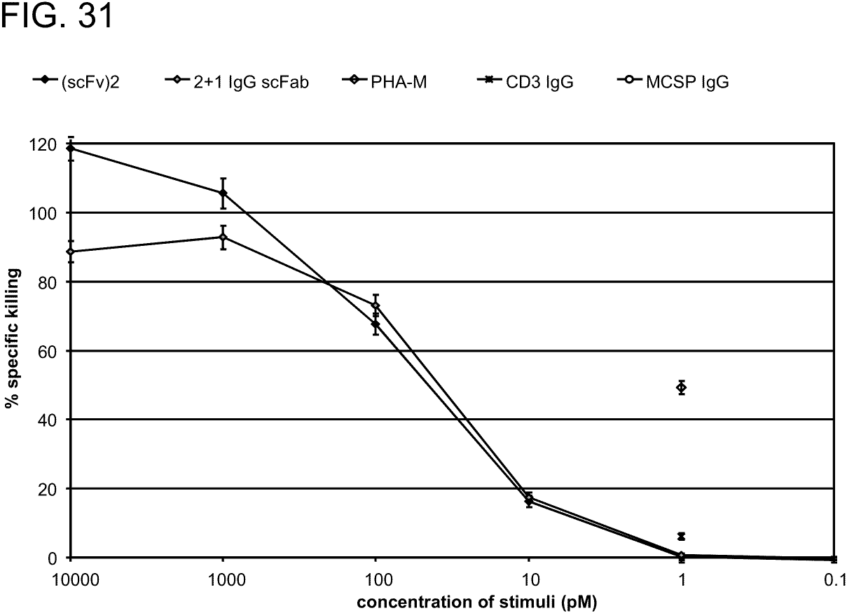

In another experiment, purified “2+1 IgG Crossfab” targeting cynomolgus CD3 and human MCSP (SEQ ID NOs 3, 5, 35, 37) was analyzed for its potential to up-regulate the surface activation marker CD25 on CD8⁺ T cells in the presence of tumor target cells. Briefly, human MCSP-expressing MV-3 tumor target cells were harvested with Cell Dissociation Buffer, washed and resuspendend in DMEM containing 2% FCS and 1% GlutaMax. 30 000 cells per well were plated in a round-bottom 96-well plate and the respective antibody dilution was added at the indicated concentrations (FIG. 25). The bispecific construct and the different IgG controls were adjusted to the same molarity. Cynomolgus PBMC effector cells, isolated from blood of two healthy animals, were added to obtain a final E:T ratio of 3:1. After an incubation for 43 h at 37° C., 5% CO₂, the cells were centrifuged at 350 × g for 5 min and washed twice with PBS, containing 0.1% BSA. Surface staining for CD8 (Miltenyi Biotech #130-080-601) and CD25 (BD #557138) was performed according to the supplier’s suggestions. Cells were washed twice with 150 µl/well PBS containing 0.1% BSA and fixed for 15 min at 4° C., using 100 µl/well fixation buffer (BD #554655). After centrifugation, the samples were resuspended in 200 µl/well PBS with 0.1% BSA and analyzed using a FACS CantoII machine (Software FACS Diva).

As depicted in FIG. 25, the bispecific construct induces concentration-dependent up-regulation of CD25 on CD8⁺ T cells only in the presence of target cells. The anti cyno CD3 IgG (clone FN-18) is also able to induce up-regulation of CD25 on CD8⁺ T cells, without being crosslinked (see data obtained with cyno Nestor). There is no hyperactivation of cyno T cells with the maximal concentration of the bispecific construct (in the absence of target cells).

In another experiment, the CD3-MCSP “2+1 IgG Crossfab, linked light chain” (see SEQ ID NOs 3, 5, 29, 179) was compared to the CD3-MCSP “2+1 IgG Crossfab” (see SEQ ID NOs 3, 5, 29, 33) for its potential to up-regulate the early activation marker CD69 or the late activation marker CD25 on CD8⁺ T cells in the presence of tumor target cells. Primary human PBMCs (isolated as described above) were incubated with the indicated concentrations of bispecific constructs for at least 22 h in the presence or absence of MCSP-positive Colo38 target cells. Briefly, 0.3 million primary human PBMCs were plated per well of a flat-bottom 96-well plate, containing the MCSP-positive target cells (or medium). The final effector to target cell (E:T) ratio was 10:1. The cells were incubated with the indicated concentration of the bispecific constructs and controls for the indicated incubation times at 37° C., 5% CO₂. The effector cells were stained for CD8, and CD69 or CD25 and analyzed by FACS CantoII.

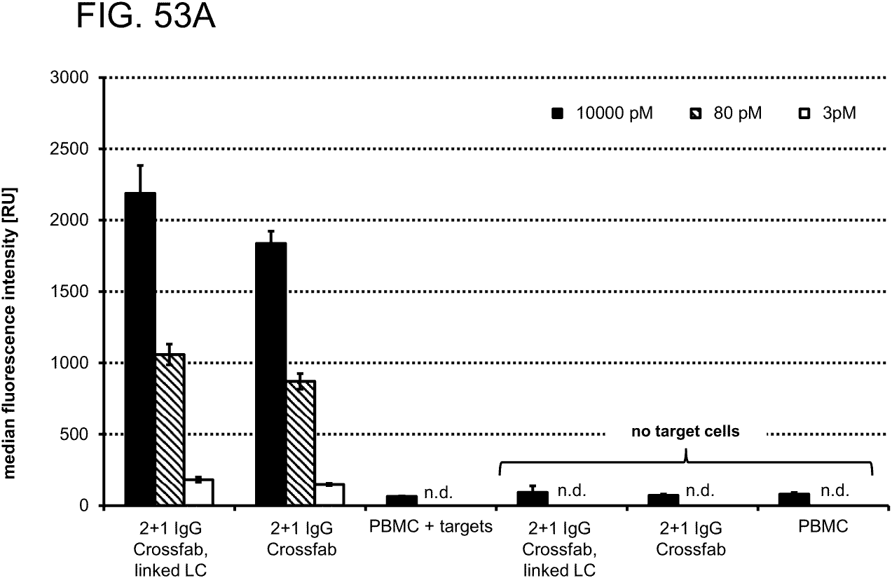

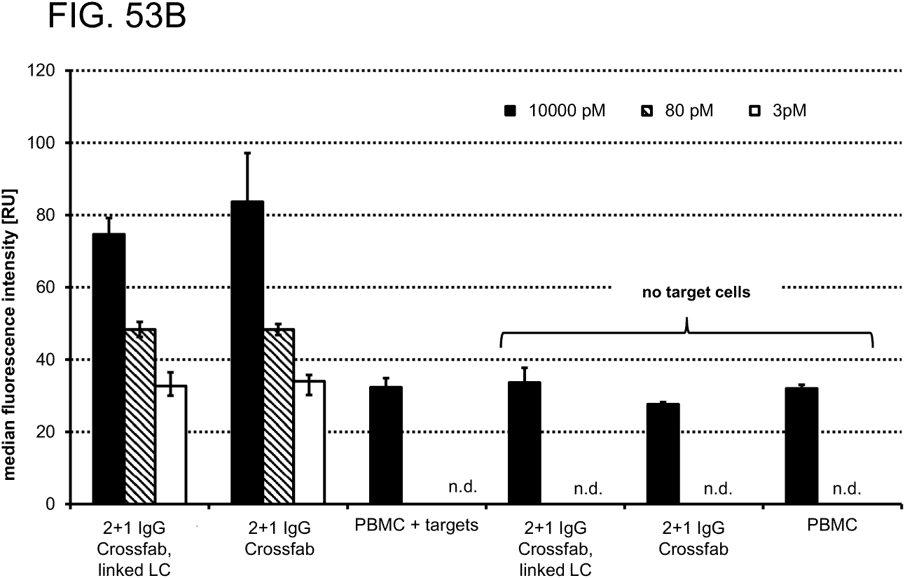

FIG. 53A and FIG. 53B showthe result of this experiment. There were no significant differences detected for CD69 (FIG. 53A) or CD25 up-regulation (FIG. 53B) between the two 2+1 IgG Crossfab molecules (with or without the linked light chain).

In yet another experiment, the CD3/MCSP “2+1 IgG Crossfab” (see SEQ ID NOs 3, 5, 29, 33) and “1+1 IgG Crossfab” (see SEQ ID NOs 5, 29, 33, 181) constructs were compared to the “1+1 CrossMab” construct (see SEQ ID NOs 5, 23, 183, 185) for their potential to up-regulate CD69 or CD25 on CD4⁺ or CD8⁺ T cells in the presence of tumor target cells. The assay was performed as described above, in the presence of absence of human MCSP expressing MV-3 tumor cells, with an incubation time of 24 h.

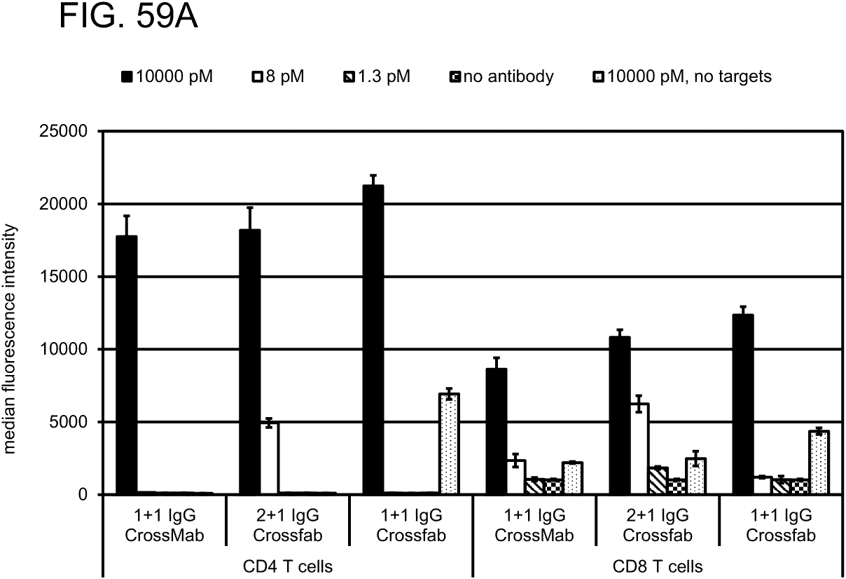

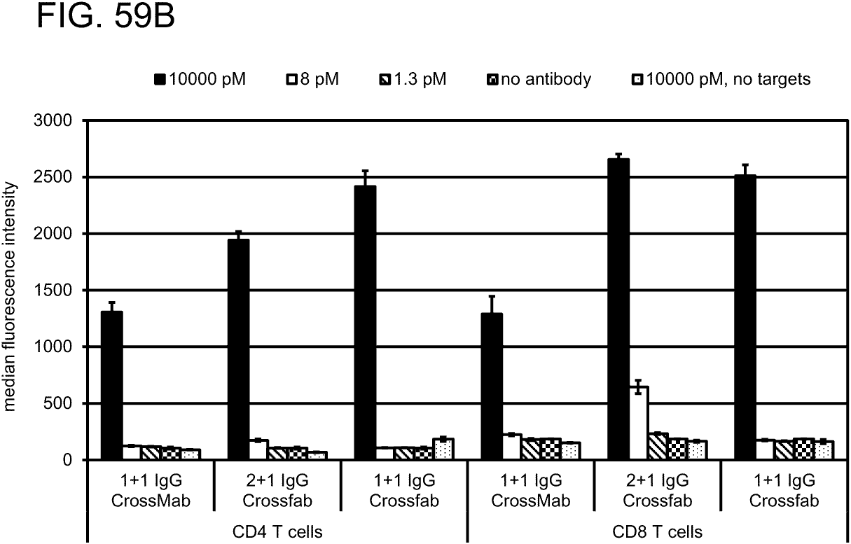

As shown in FIG. 59A and FIG. 59B, the “1+1 IgG Crossfab” and “2+1 IgG Crossfab” constructs induced more pronounced upregulation of activation markers than the “1+1 CrossMab” molecule.

In a final experiment, the CD3/MCSP “2+1 IgG Crossfab” (see SEQ ID NOs 5, 23, 215, 217) and “2+1 IgG Crossfab, inverted” (see SEQ ID NOs 5, 23, 215, 219) constructs were assessed for their potential to up-regulate CD25 on CD4⁺ or CD8⁺ T cells from two different cynomolgus monkeys in the presence of tumor target cells. The assay was performed as described above, in the presence of absence of human MCSP expressing MV-3 tumor cells, with an E:T ratio of 3:1 and an incubation time of about 41 h.

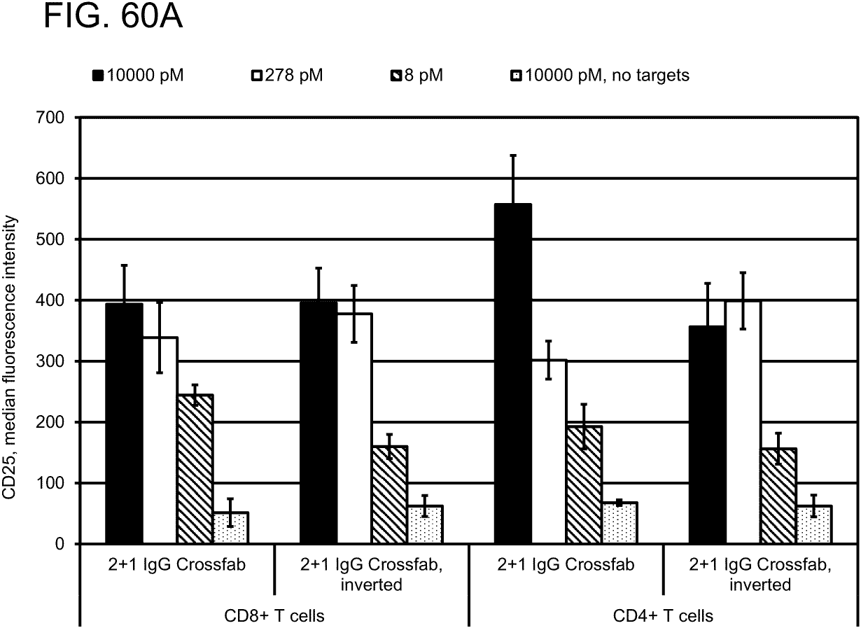

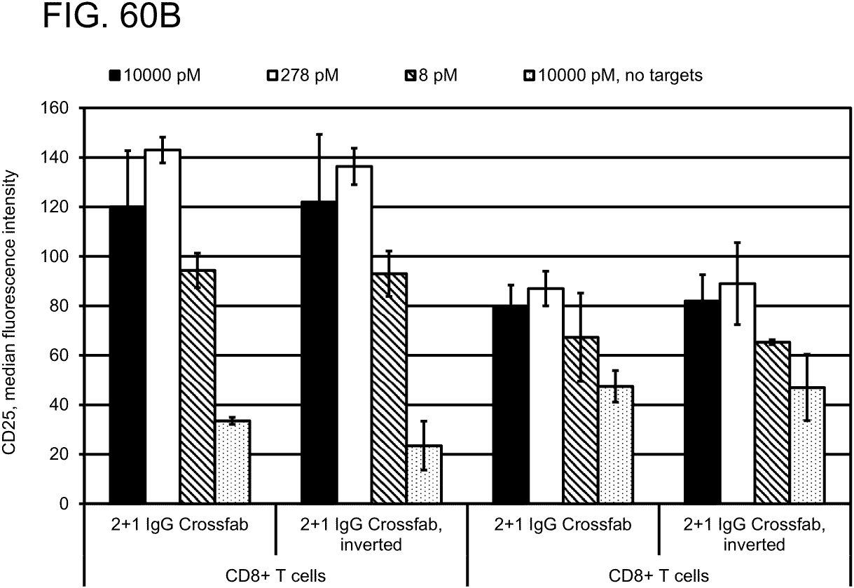

As shown in FIG. 60A and FIG. 60B, both constructs were able to up-regulate CD25 on CD4⁺ and CD8⁺ T cells in a concentration-dependent manner, without significant difference between the two formats. Control samples without antibody and without target cells gave a comparable signal to the samples with antibody but no targets (not shown).

### Example 5 Interferon-y Secretion Upon Activation of Human Pan T Cells with CD3 Bispecific Constructs

Purified “2+1 IgG scFab” targeting human MCSP and human CD3 (SEQ ID NOs 5, 17, 19) was analyzed for its potential to induce T cell activation in the presence of human MCSP-positive U-87MG cells, measured by the release of human interferon (IFN)-γ into the supernatant. As controls, anti-human MCSP and anti-human CD3 IgGs were used, adjusted to the same molarity. Briefly, huMCSP-expressing U-87MG glioblastoma astrocytoma target cells (ECACC 89081402) were harvested with Cell Dissociation Buffer, washed and resuspendend in AIM-V medium (Invitrogen #12055-091). 20 000 cells per well were plated in a round-bottom 96-well-plate and the respective antibody dilution was added to obtain a final concentration of 1 nM. Human pan T effector cells, isolated from Buffy Coat, were added to obtain a final E:T ratio of 5:1. After an overnight incubation of 18.5 h at 37° C., 5% CO₂, the assay plate was centrifuged for 5 min at 350 × g and the supernatant was transferred into a fresh 96-well plate. Human IFN-γ levels in the supernatant were measured by ELISA, according to the manufacturer’s instructions (BD OptEIA human IFN-γ ELISA Kit II from Becton Dickinson, #550612).

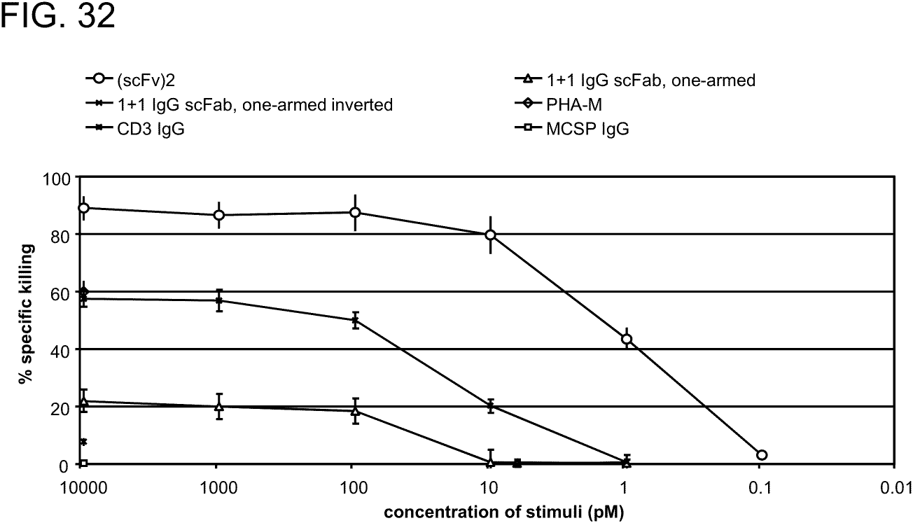

As depicted in FIG. 26, the reference IgGs show no to weak induction of IFN-γ secretion, whereas the “2+1 IgG scFab” construct is able to activate human T cells to secrete IFN-γ.

### Example 6 Re-directed T Cell Cytotoxicity Mediated by Cross-linked Bispecific Constructs Targeting CD3 on T Cells and MCSP or EGFR on Tumor Cells (LDH release assay)

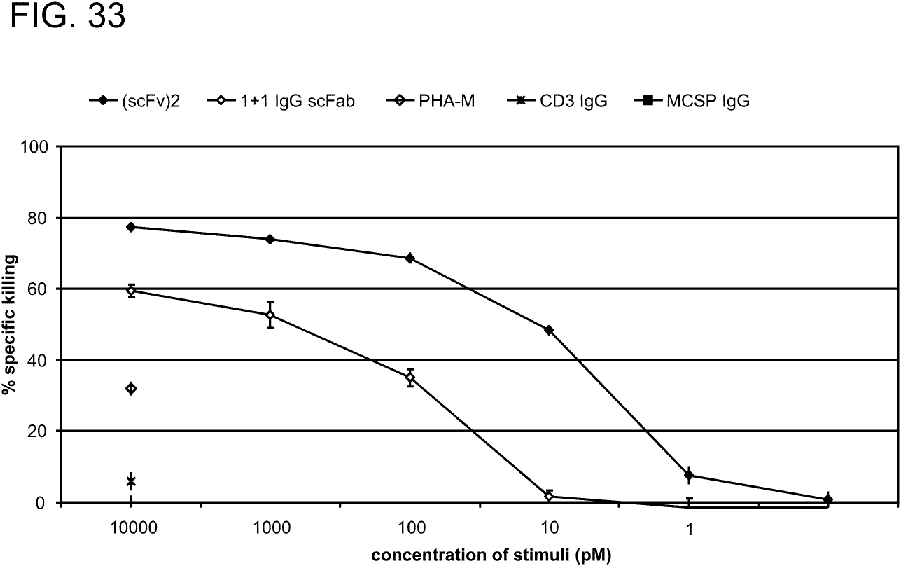

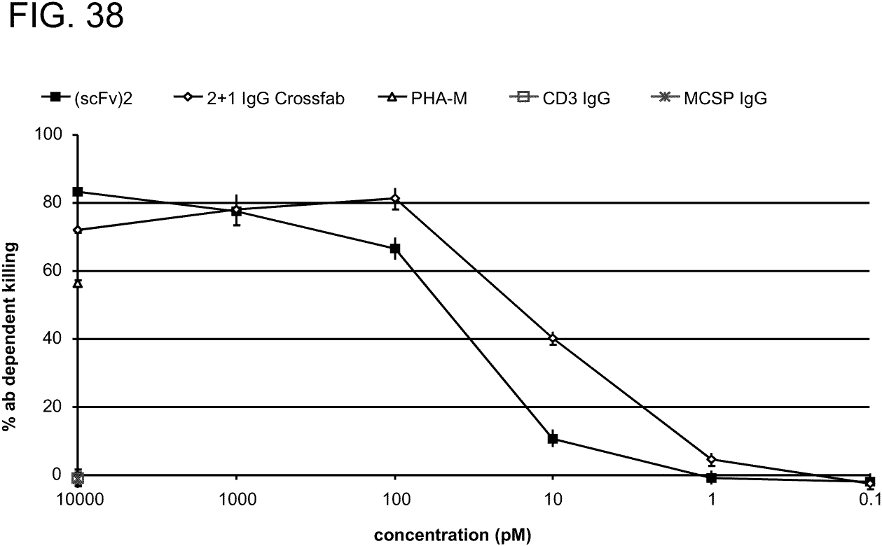

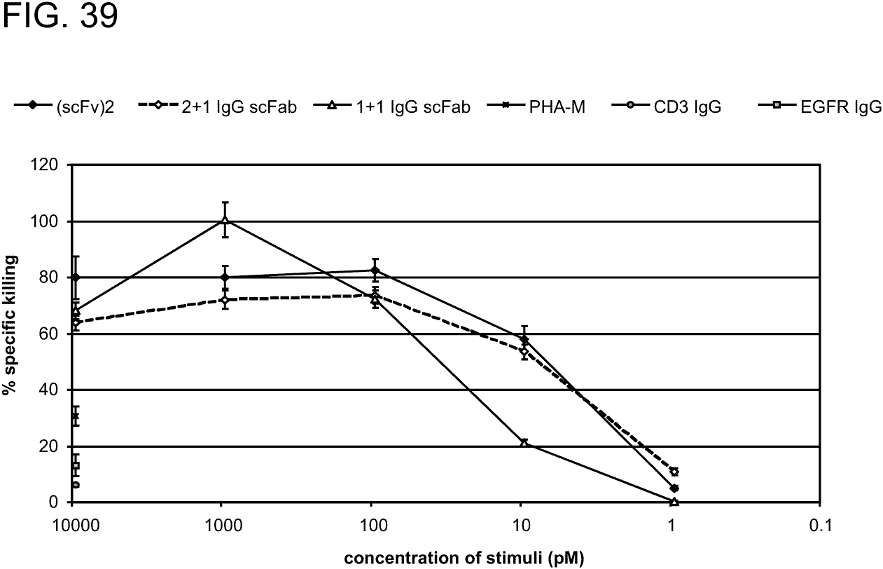

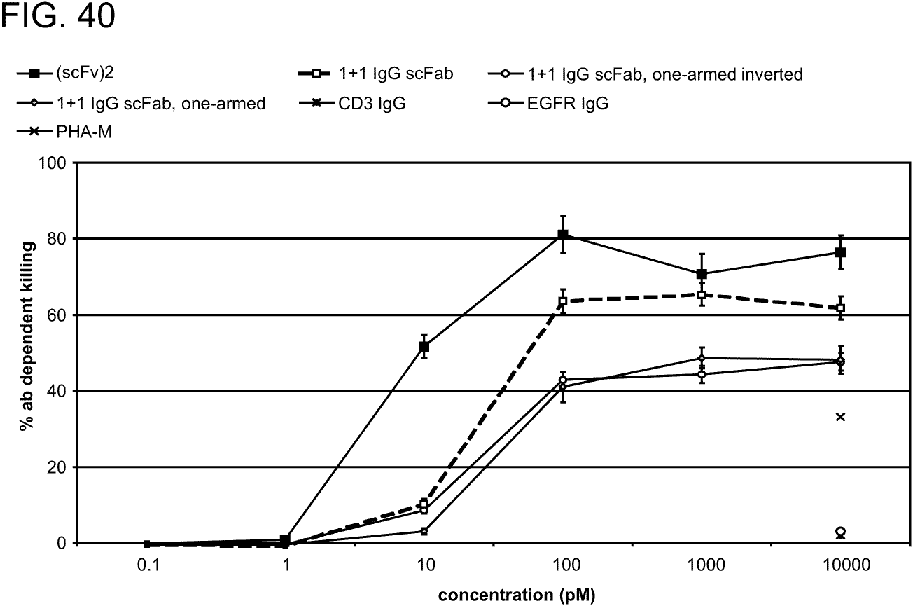

In a first series of experiments, bispecific constructs targeting CD3 and MCSP were analyzed for their potential to induce T cell-mediated apoptosis in tumor target cells upon crosslinkage of the construct via binding of the antigen binding moieties to their respective target antigens on cells (FIG. 27- FIG. 38).

In one experiment purified “2+1 IgG scFab” (SEQ ID NOs 5, 21, 23) and “2+1 IgG Crossfab” (SEQ ID NOs 3, 5, 29, 33) constructs targeting human CD3 and human MCSP, and the corresponding “(scFv)₂” molecule, were compared. Briefly, huMCSP-expressing MDA-MB-435 human melanoma target cells were harvested with Cell Dissociation Buffer, washed and resuspendend in AIM-V medium (Invitrogen # 12055-091). 30 000 cells per well were plated in a round-bottom 96-well plate and the respective dilution of the construct was added at the indicated concentration. All constructs and corresponding control IgGs were adjusted to the same molarity. Human pan T effector cells were added to obtain a final E:T ratio of 5:1. As a positive control for the activation of human pan T cells, 1 µg/ml PHA-M (Sigma #L8902; mixture of isolectins isolated from Phaseolus vulgaris) was used. For normalization, maximal lysis of the target cells (= 100%) was determined by incubation of the target cells with a final concentration of 1% Triton X-100. Minimal lysis (= 0%) refers to target cells co-incubated with effector cells, but without any construct or antibody. After an overnight incubation of 20 h at 37° C., 5% CO₂, LDH release of apoptotic/necrotic target cells into the supernatant was measured with the LDH detection kit (Roche Applied Science, #11 644 793 001), according to the manufacturer’s instructions.

As depicted in FIG. 27, both “2+1” constructs induce apoptosis in target cells comparable to the “(scFv)₂” molecule.

Further, purified “2+1 IgG Crossfab” (SEQ ID NOs 3, 5, 29, 33) and “2+1 IgG scFab” constructs differing in their Fc domain, as well as the “(scFv)₂” molecule, were compared. The different mutations in the Fc domain (L234A+L235A (LALA), P329G and/or N297D, as indicated) reduce or abolish the (NK) effector cell function induced by constructs containing a wild-type (wt) Fc domain. Experimental procedures were as described above.

FIG. 28 shows that all constructs induce apoptosis in target cells comparable to the “(scFv)₂” molecule.

FIG. 29 shows the result of a comparison of the purified “2+1 IgG scFab” (SEQ ID NOs 5, 17, 19) and the “(scFv)₂” molecule for their potential to induce T cell-mediated apoptosis in tumor target cells. Experimental procedures were as decribed above, using huMCSP-expressing Colo-38 human melanoma target cells at an E:T ratio of 5:1, and an overnight incubation of 18.5 h. As depicted in the figure, the “2+1 IgG scFab” construct shows comparable cytotoxic activity to the “(scFv)₂” molecule.

Similarly, FIG. 30 shows the result of a comparison of the purified “2+1 IgG scFab” construct (SEQ ID NOs 5, 17, 19)and the “(scFv)₂” molecule, using huMCSP-expressing Colo-38 human melanoma target cells at an E:T ratio of 5:1 and an incubation time of 18 h. As depicted in the figure, the “2+1 IgG scFab” construct shows comparable cytotoxic activity to the (scFv)₂ molecule.

FIG. 31 shows the result of a comparison of the purified “2+1 IgG scFab” construct (SEQ ID NOs 5, 17, 19) and the “(scFv)₂” molecule, using huMCSP-expressing MDA-MB-435 human melanoma target cells at an E:T ratio of 5:1 and an overnight incubation of 23.5 h. As depicted in the figure, the construct induces apoptosis in target cells comparably to the “(scFv)₂” molecule. The “2+1 IgG scFab” construct shows reduced efficacy at the highest concentrations.

Furthermore, different bispecific constructs that are monovalent for both targets, human CD3 and human MCSP, as well as the corresponding “(scFv)₂” molecule were analyzed for their potential to induce T cell-mediated apoptosis. FIG. 32 shows the results for the “1+1 IgG scFab, one-armed” (SEQ ID NOs 1, 3, 5) and “1+1 IgG scFab, one-armed inverted” (SEQ ID NOs 7, 9, 11) constructs, using huMCSP-expressing Colo-38 human melanoma target cells at an E:T ratio of 5:1, and an incubation time of 19 h. As depicted in the figure, both “1+1” constructs are less active than the “(scFv)₂” molecule, with the “1+1 IgG scFab, one-armed” molecule being superior to the “1+1 IgG scFab, one-armed inverted” molecule in this assay.

FIG. 33 shows the results for the “1+1 IgG scFab” construct (SEQ ID NOs 5, 21, 213), using huMCSP-expressing Colo-38 human melanoma target cells at an E:T ratio of 5:1, and an incubation time of 20 h. As depicted in the figure, the “1+1 IgG scFab” construct is less cytotoxic than the “(scFv)₂” molecule.

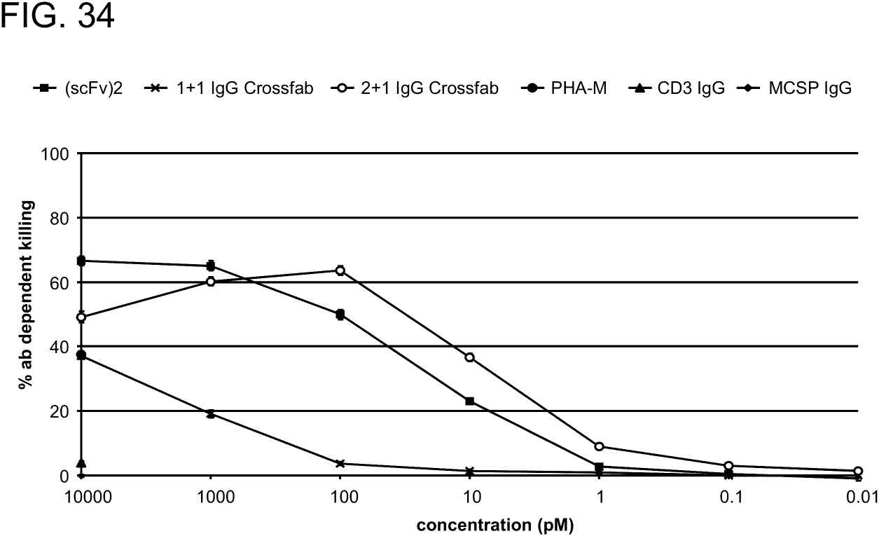

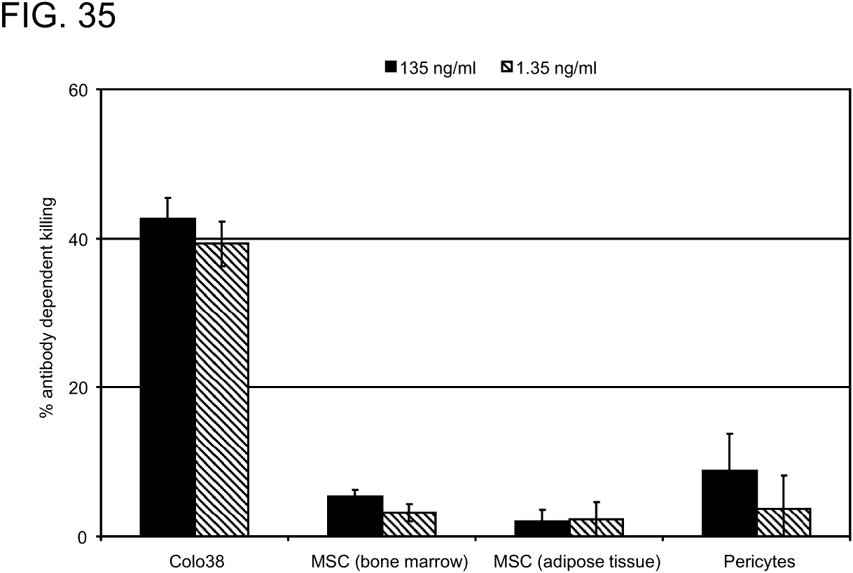

In a further experiment the purified “2+1 IgG Crossfab” (SEQ ID NOs 3, 5, 29, 33), the “1+1 IgG Crossfab” (SEQ ID NOs 5, 29, 31, 33) and the “(scFv)₂” molecule were analyzed for their potential to induce T cell-mediated apoptosis in tumor target cells upon crosslinkage of the construct via binding of both target antigens, CD3 and MCSP, on cells. huMCSP-expressing MDA-MB-435 human melanoma cells were used as target cells, the E:T ratio was 5:1, and the incubation time 20 h. The results are shown in FIG. 34. The “2+1 IgG Crossfab” construct induces apoptosis in target cells comparably to the “(scFv)₂” molecule. The comparison of the mono- and bivalent “IgG Crossfab” formats clearly shows that the bivalent one is much more potent.

In yet another experiment, the purified “2+1 IgG Crossfab” (SEQ ID NOs 3, 5, 29, 33) construct was analyzed for its potential to induce T cell-mediated apoptosis in different (tumor) target cells. Briefly, MCSP-positive Colo-38 tumor target cells, mesenchymal stem cells (derived from bone marrow, Lonza #PT-2501 or adipose tissue, Invitrogen #R7788-115) or pericytes (from placenta; PromoCell #C-12980), as indicated, were harvested with Cell Dissociation Buffer, washed and resuspendend in AIM-V medium (Invitrogen #12055-091). 30 000 cells per well were plated in a round-bottom 96-well plate and the respective antibody dilution was added at the indicated concentrations. Human PBMC effector cells isolated from fresh blood of a healthy donor were added to obtain a final E:T ratio of 25:1. After an incubation of 4 h at 37° C., 5% CO₂, LDH release of apoptotic/necrotic target cells into the supernatant was measured with the LDH detection kit (Roche Applied Science, #11 644 793 001), according to the manufacturer’s instructions.

As depicted in FIG. 35, significant T-cell mediated cytotoxicity could be observed only with Colo-38 cells. This result is in line with Colo-38 cells expressing significant levels of MCSP, whereas mesenchymal stem cells and pericytes express MCSP only very weakly.

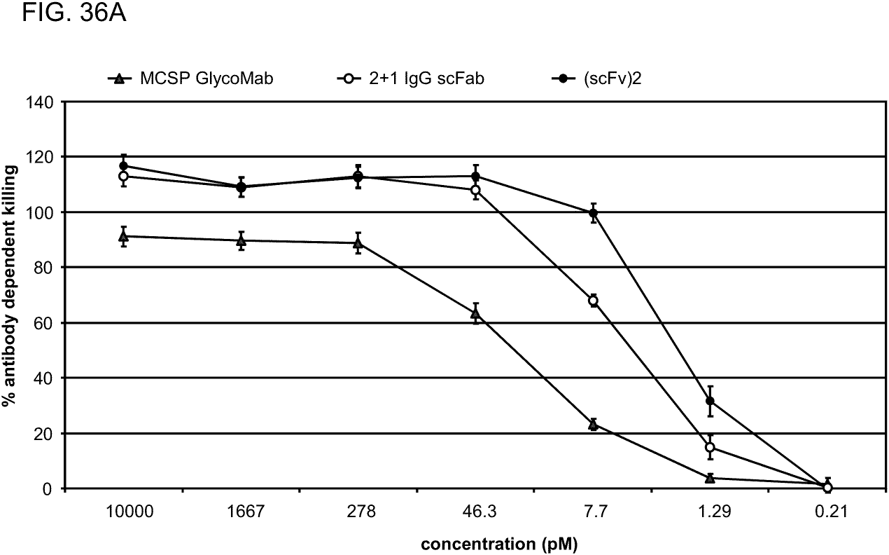

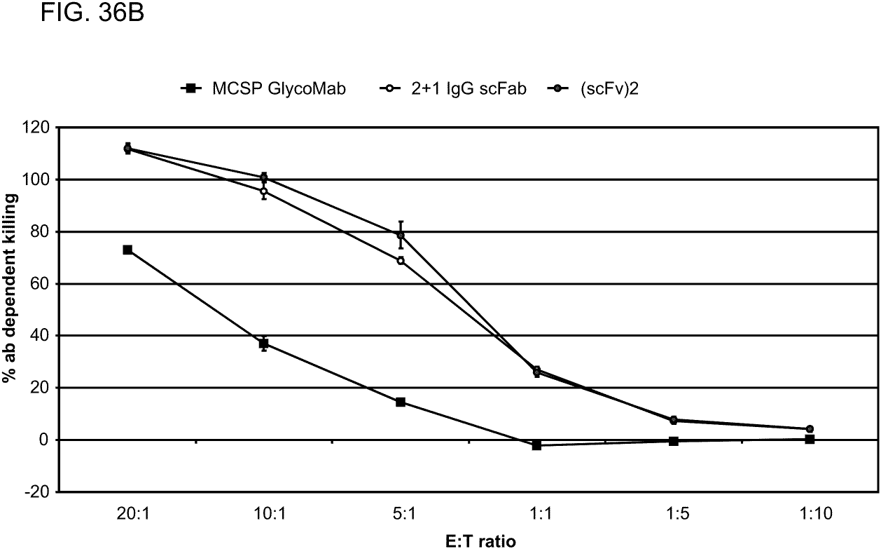

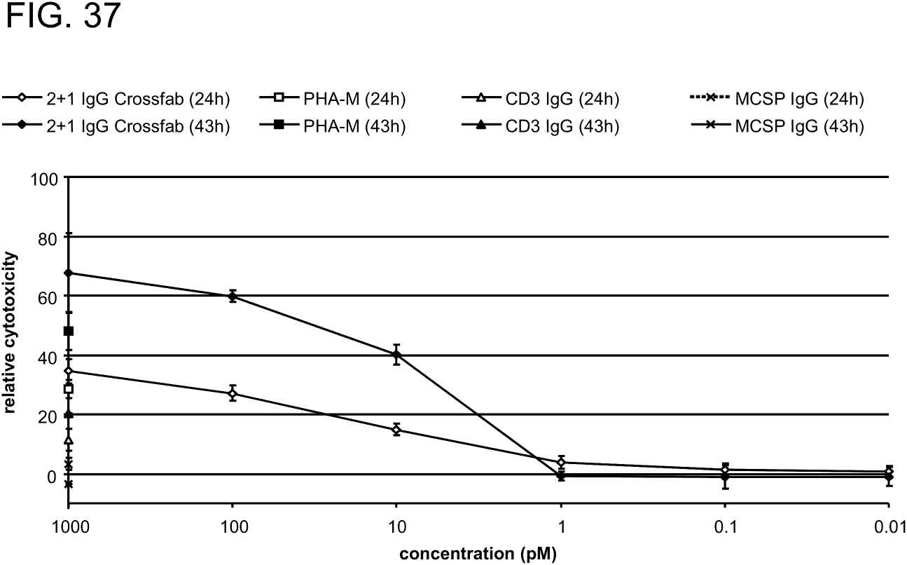

The purified “2+1 IgG scFab” (SEQ ID NOs 5, 17, 19) construct and the “(scFv)₂” molecule were also compared to a glycoengineered anti-human MCSP IgG antibody, having a reduced proportion of fucosylated N-glycans in its Fc domain (MCSP GlycoMab). For this experiment huMCSP-expressing Colo-38 human melanoma target cells and human PBMC effector cells were used, either at a fixed E:T ratio of 25:1 (FIG. 36A), or at different E:T ratios from 20:1 to 1:10 (FIG. 36B). The different molecules were used at the concentrations indicated in FIG. 36A, or at a fixed concentration of 1667 pM (FIG. 36B). Read-out was done after 21 h incubation. As depicted in FIG. 36A and FIG. 36B, both bispecific constructs show a higher potency than the MSCP GlycoMab.

In another experiment, purified “2+1 IgG Crossfab” targeting cynomolgus CD3 and human MCSP (SEQ ID NOs 3, 5, 35, 37) was analyzed. Briefly, human MCSP-expressing MV-3 tumor target cells were harvested with Cell Dissociation Buffer, washed and resuspendend in DMEM containing 2% FCS and 1% GlutaMax. 30 000 cells per well were plated in a round-bottom 96-well plate and the respective dilution of construct or reference IgG was added at the concentrations indicated. The bispecific construct and the different IgG controls were adjusted to the same molarity. Cynomolgus PBMC effector cells, isolated from blood of healthy cynomolgus, were added to obtain a final E:T ratio of 3:1. After incubation for 24 h or 43 h at 37° C., 5% CO₂, LDH release of apoptotic/necrotic target cells into the supernatant was measured with the LDH detection kit (Roche Applied Science, #11 644 793 001), according to the manufacturer’s instructions.

As depicted in FIG. 37, the bispecific construct induces concentration-dependent LDH release from target cells. The effect is stronger after 43 h than after 24 h. The anti-cynoCD3 IgG (clone FN-18) is also able to induce LDH release of target cells without being crosslinked.

FIG. 38 shows the result of a comparison of the purified “2+1 IgG Crossfab” (SEQ ID NOs 3, 5, 29, 33) and the “(scFv)₂” construct, using MCSP-expressing human melanoma cell line (MV-3) as target cells and human PBMCs as effector cells with an E:T ratio of 10:1 and an incubation time of 26 h. As depicted in the figure, the “2+1 IgG Crossfab” construct is more potent in terms of EC50 than the “(scFv)₂” molecule.

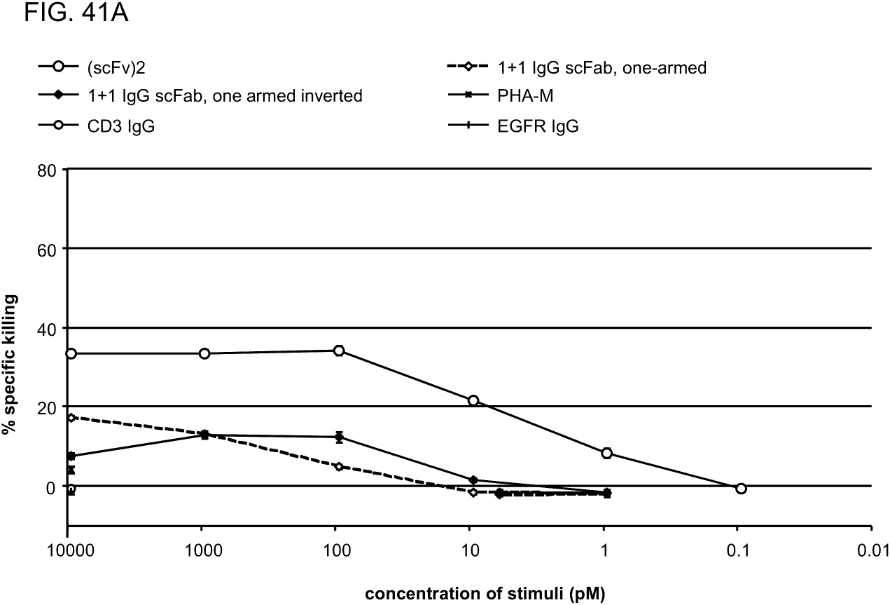

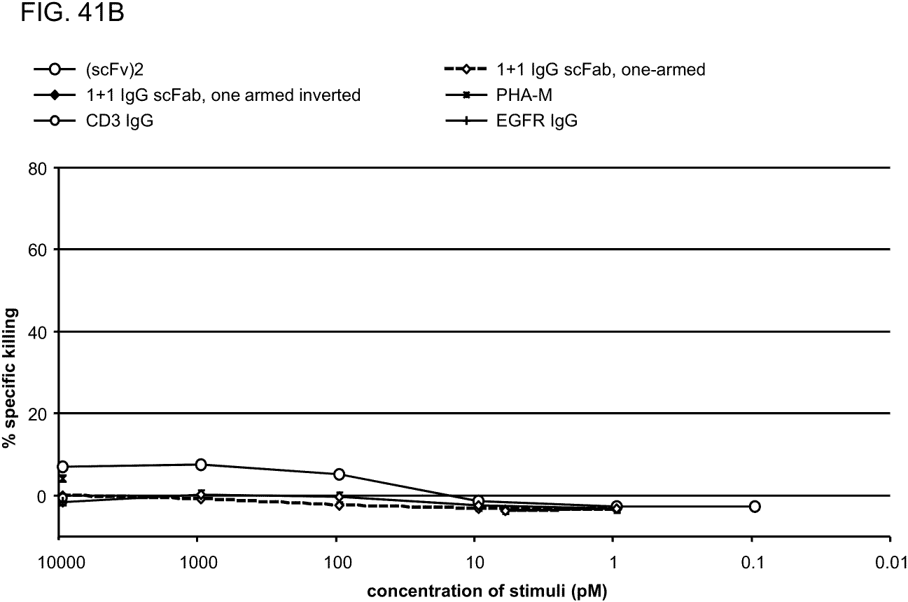

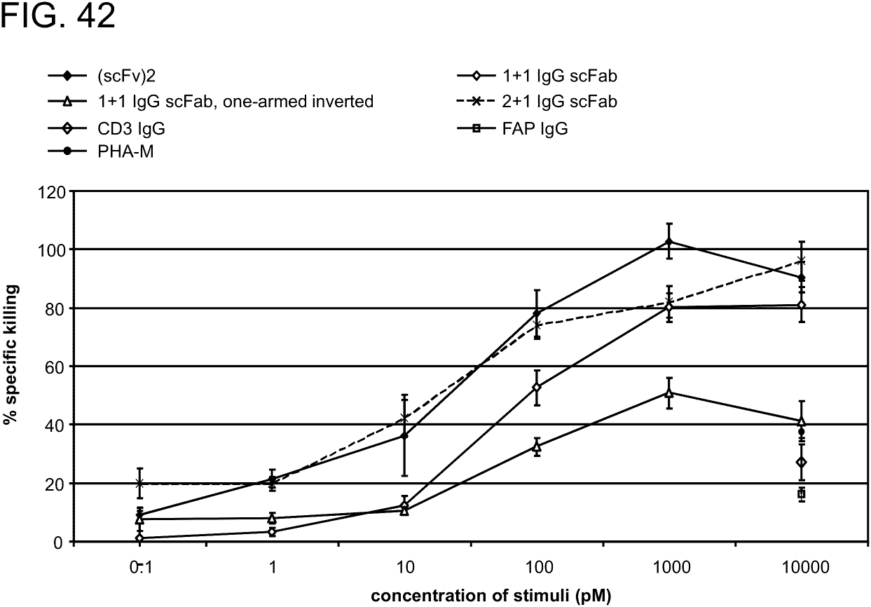

In a second series of experiments, bispecific constructs targeting CD3 and EGFR were analyzed for their potential to induce T cell-mediated apoptosis in tumor target cells upon crosslinkage of the construct via binding of the antigen binding moieties to their respective target antigens on cells (FIG. 39 - FIG. 41).

In one experiment purified “2+1 IgG scFab” (SEQ ID NOs 45, 47, 53) and “1+1 IgG scFab” (SEQ ID NOs 47, 53, 213) constructs targeting CD3 and EGFR, and the corresponding “(scFv)₂” molecule, were compared. Briefly, human EGFR-expressing LS-174T tumor target cells were harvested with trypsin, washed and resuspendend in AIM-V medium (Invitrogen # 12055-091). 30 000 cells per well were plated in a round-bottom 96-well-plate and the respective antibody dilution was added at the indicated concentrations. All constructs and controls were adjusted to the same molarity. Human pan T effector cells were added to obtain a final E:T ratio of 5:1. As a positive control for the activation of human pan T cells, 1 µg/ml PHA-M (Sigma #L8902) was used. For normalization, maximal lysis of the target cells (= 100%) was determined by incubation of the target cells with a final concentration of 1% Triton X-100. Minimal lysis (= 0%) refers to target cells co-incubated with effector cells, but without any construct or antibody. After an overnight incubation of 18 h at 37° C., 5% CO₂, LDH release of apoptotic/necrotic target cells into the supernatant was measured with the LDH detection kit (Roche Applied Science, #11 644 793 001), according to the manufacturer’s instructions.

As depicted in FIG. 39, the “2+1 IgG scFab” construct shows comparable cytotoxic activity to the “(scFv)₂” molecule, whereas the “1+1 IgG scFab” construct is less active.

In another experiment the purified “1+1 IgG scFab, one-armed” (SEQ ID NOs 43, 45, 47), “1+1 IgG scFab, one-armed inverted” (SEQ ID NOs 11, 49, 51), “1+1 IgG scFab” (SEQ ID NOs 47, 53, 213), and the “(scFv)₂” molecule were compared. Experimental conditions were as described above, except for the incubation time which was 21 h.

As depicted in FIG. 40, the “1+1 IgG scFab” construct shows a slightly lower cytotoxic activity than the “(scFv)₂” molecule in this assay. Both “1+1 IgG scFab, one-armed (inverted)” constructs are clearly less active than the “(scFv)₂” molecule.

In yet a further experiment the purified “1+1 IgG scFab, one-armed” (SEQ ID NO 43, 45, 47) and “1+1 IgG scFab, one-armed inverted” (SEQ ID NOs 11, 49, 51) constructs and the “(scFv)₂” molecule were compared. The incubation time in this experiment was 16 h, and the result is depicted in FIG. 41A and FIG. 41B. Incubated with human pan T cells, both “1+1 IgG scFab, one-armed (inverted)” constructs are less active than the “(scFv)₂” molecule, but show concentration-dependent release of LDH from target cells (FIG. 41A). Upon co-cultivation of the LS-174T tumor cells with naive T cells isolated from PBMCs, the constructs had only a basal activity - the most active among them being the “(scFv)₂” molecule (FIG. 41B).

In a further experiment, purified “1+1 IgG scFab, one-armed inverted” (SEQ ID NOs 11, 51, 55), “1+1 IgG scFab” (57, 61, 213), and “2+1 IgG scFab” (57, 59, 61) targeting CD3 and Fibroblast Activation Protein (FAP), and the corresponding “(scFv)₂” molecule were analyzed for their potential to induce T cell-mediated apoptosis in human FAP-expressing fibroblasts GM05389 cells upon crosslinkage of the construct via binding of both targeting moieties to their respective target antigens on the cells. Briefly, human GM05389 target cells were harvested with trypsin on the day before, washed and resuspendend in AIM-V medium (Invitrogen #12055-091). 30 000 cells per well were plated in a round-bottom 96-well plate and incubated overnight at 37° C., 5% CO₂ to allow the cells to recover and adhere. The next day, the cells were centrifuged, the supernatant was discarded and fresh medium, as well as the respective dilution of the constructs or reference IgGs was added at the indicated concentrations. All constructs and controls were adjusted to the same molarity. Human pan T effector cells were added to obtain a final E:T ratio of 5:1. As a positive control for the activation of human pan T cells, 5 µg/ml PHA-M (Sigma #L8902) was used. For normalization, maximal lysis of the target cells (= 100%) was determined by incubation of the target cells with a final concentration of 1% Triton X-100. Minimal lysis (= 0%) refers to target cells co-incubated with effector cells, but without any construct or antibody. After an additional overnight incubation of 18 h at 37° C., 5% CO₂, LDH release of apoptotic/necrotic target cells into the supernatant was measured with the LDH detection kit (Roche Applied Science, #11 644 793 001), according to the manufacturer’s instructions.

As depicted in FIG. 42, the “2+1 IgG scFab” construct shows comparable cytotoxic activity to the “(scFv)₂” molecule in terms of EC50 values. The “1+1 IgG scFab, one-armed inverted” construct is less active than the other constructs tested in this assay.

In another set of experiments, the CD3/MCSP “2+1 IgG Crossfab, linked light chain” (see SEQ ID NOs 3, 5, 29, 179) was compared to the CD3/MCSP “2+1 IgG Crossfab” (see SEQ ID NOs 3, 5, 29, 33). Briefly, target cells (human Colo-38, human MV-3 or WM266-4 melanoma cells) were harvested with Cell Dissociation Buffer on the day of the assay (or with trypsin one day before the assay was started), washed and resuspended in the appropriate cell culture medium (RPMI1640, including 2% FCS and 1% Glutamax). 20 000 - 30 000 cells per well were plated in a flat-bottom 96-well plate and the respective antibody dilution was added as indicated (triplicates). PBMCs as effector cells were added to obtain a final effector-to-target cell (E:T) ratio of 10:1. All constructs and controls were adjusted to the same molarity, incubation time was 22 h. Detection of LDH release and normalization was done as described above.

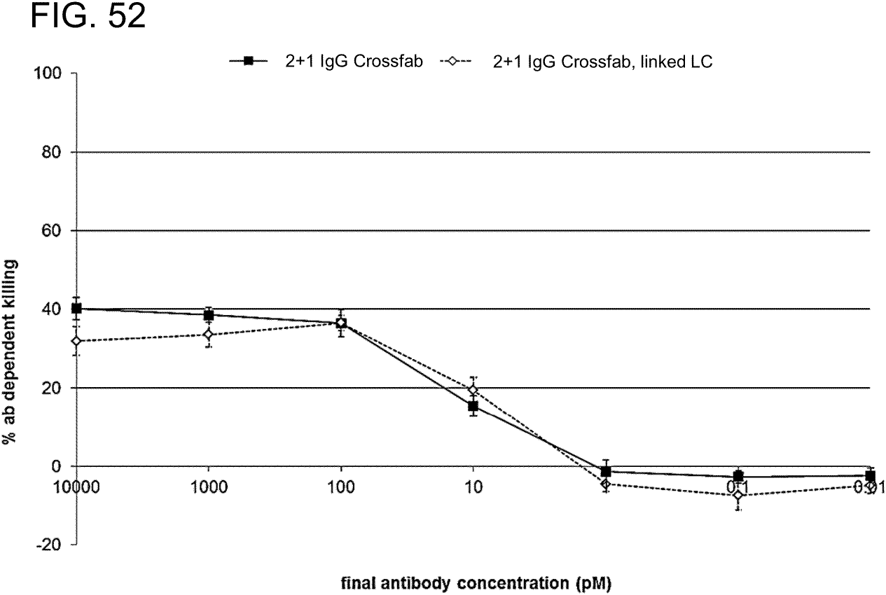

FIG. 49 to FIG. 52 show the result of four assays performed with MV-3 melanoma cells (FIG. 49), Colo-38 cells (FIG. 50 and FIG. 51) or WM266-4 cells (FIG. 52). As shown in FIG. 49, the construct with the linked light chain was less potent compared to the one without the linked light chain in the assay with MV-3 cells as target cells. As shown in FIG. 50 and FIG. 51, the construct with the linked light chain was more potent compared to the one without the linked light chain in the assays with high MCSP expressing Colo-38 cells as target cells. Finally, as shown in FIG. 52, there was no significant difference between the two constructs when high MCSP-expressing WM266-4 cells were used as target cells.

In another experiment, two CEA-targeting “2+1 IgG Crossfab, inverted” constructs were compared, wherein in the Crossfab fragment either the V regions (VL/VH, see SEQ ID NOs 33, 63, 65, 67) or the C regions (CL/CH1, see SEQ ID NOs 65, 67, 183, 197) were exchanged. The assay was performed as described above, using human PBMCs as effector cells and human CEA-expressing target cells. Target cells (MKN-45 or LS-174T tumor cells) were harvested with trypsin-EDTA (LuBiosciences #25300-096), washed and resuspendend in RPMI1640 (Invitrogen #42404042), including 1% Glutamax (LuBiosciences #35050087) and 2% FCS. 30 000 cells per well were plated in a round-bottom 96-well plate and the bispecific constructs were added at the indicated concentrations. All constructs and controls were adjusted to the same molarity. Human PBMC effector cells were added to obtain a final E:T ratio of 10:1, incubation time was 28 h. EC50 values were calculated using the GraphPad Prism 5 software.

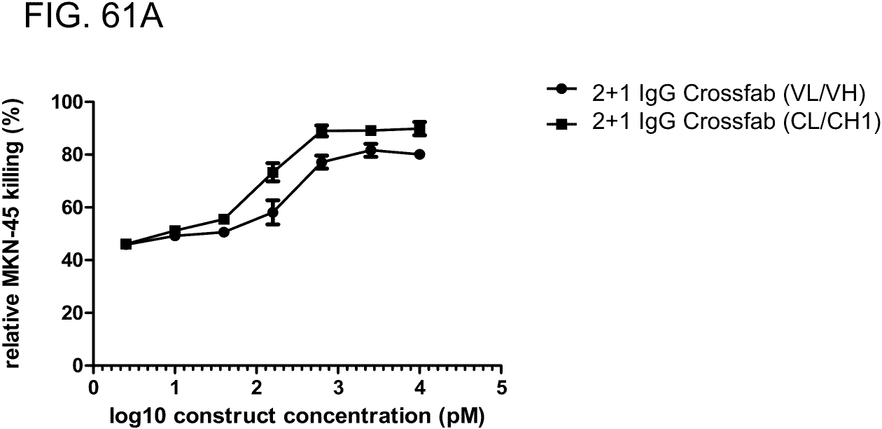

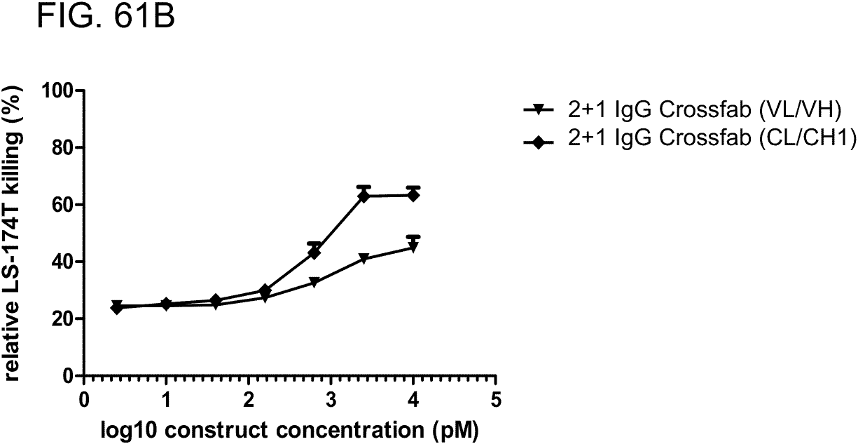

As shown in FIG. 61A and FIG. 61B, the construct with the CL/CH1 exchange shows slightly better activity on both target cell lines than the construct with the VL/VH exchange. Calculated EC50 values were 115 and 243 pM on MKN-45 cells, and 673 and 955 pM on LS-174T cells, for the CL/CH1-exchange construct and the VL/VH-exchange construct, respectively.

Similarly, two MCSP-targeting “2+1 IgG Crossfab” constructs were compared, wherein in the Crossfab fragment either the V regions (VL/VH, see SEQ ID NOs 33, 189, 191, 193) or the C regions (CL/CH1, see SEQ ID NOs 183, 189, 193, 195) were exchanged. The assay was performed as described above, using human PBMCs as effector cells and human MCSP-expressing target cells. Target cells (WM266-4) were harvested with Cell Dissociation Buffer (LuBiosciences #13151014), washed and resuspendend in RPMI1640 (Invitrogen #42404042), including 1% Glutamax (LuBiosciences #35050087) and 2% FCS. 30 000 cells per well were plated in a round-bottom 96-well plate and the constructs were added at the indicated concentrations. All constructs and controls were adjusted to the same molarity. Human PBMC effector cells were added to obtain a final E:T ratio of 10:1, incubation time was 26 h. EC50 values were calculated using the GraphPad Prism 5 software.

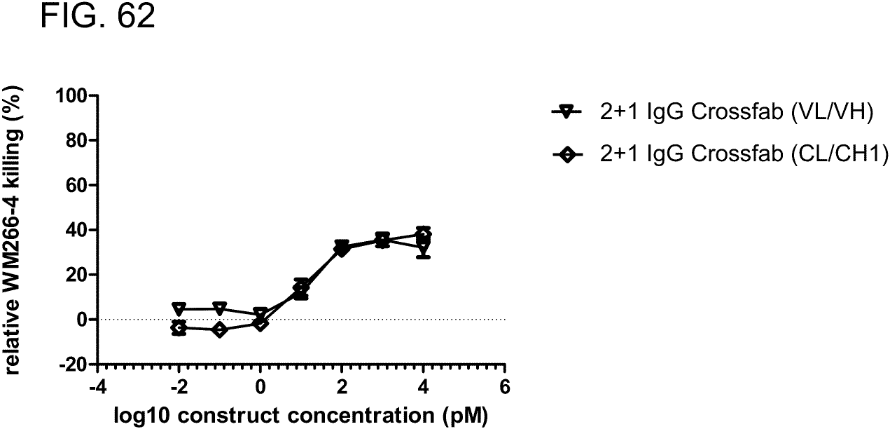

As depicted in FIG. 62, the two constructs show comparable activity, the construct with the CL/CH1 exchange having a slightly lower EC50 value (12.9 pM for the CL/CH1-exchange construct, compared to 16.8 pM for the VL/VH-exchange construct).

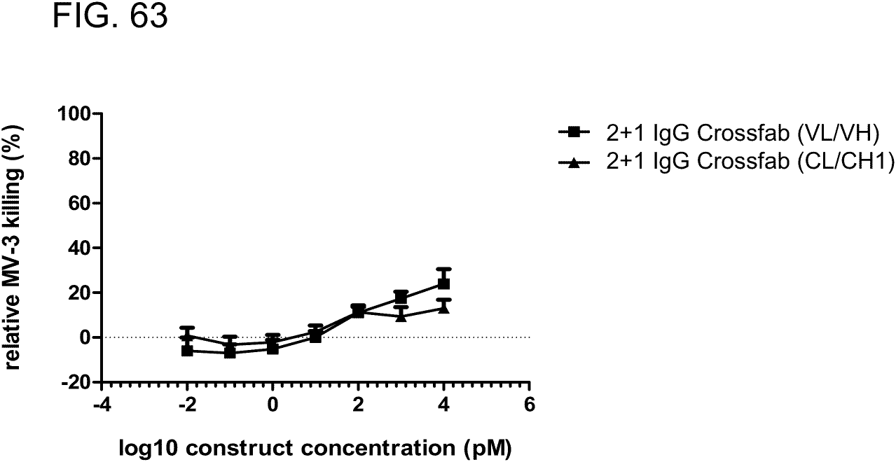

FIG. 63 shows the result of a similar assay, performed with human MCSP-expressing MV-3 target cells. Again, both constructs show comparable activity, the construct with the CL/CH1 exchange having a slightly lower EC50 value (approximately 11.7 pM for the CL/CH1-exchange construct, compared to approximately 82.2 pM for the VL/VH-exchange construct). Exact EC50 values could not be calculated, since the killing curves did not reach a plateau at high concentrations of the compounds.

In a further experiment, the CD3/MCSP “2+1 IgG Crossfab” (see SEQ ID NOs 3, 5, 29, 33) and “1+1 IgG Crossfab” (see SEQ ID NOs 5, 29, 33, 181) constructs were compared to the CD3/MCSP “1+1 CrossMab” (see SEQ ID NOs 5, 23, 183, 185). The assay was performed as described above, using human PBMCs as effector cells and WM266-4 or MV-3 target cells (E:T ratio = 10:1) and an incubation time of 21 h.

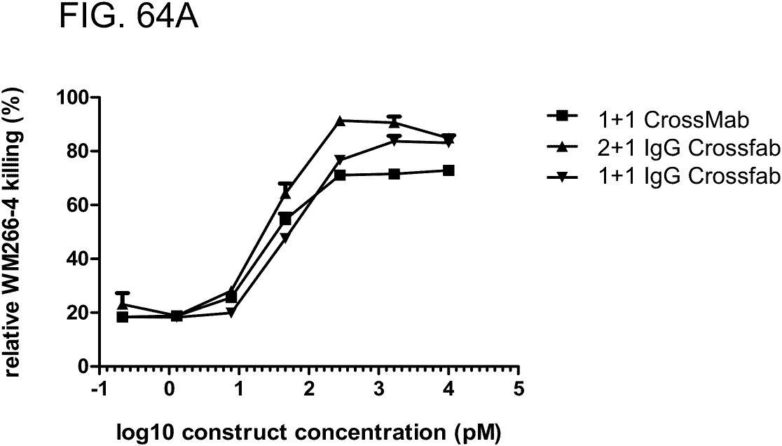

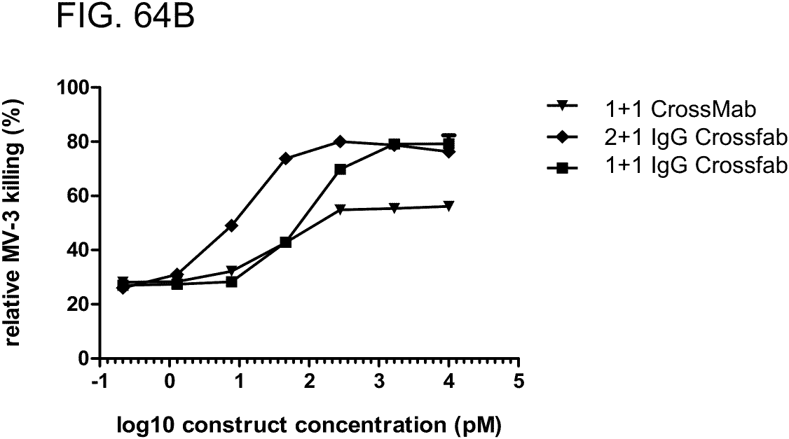

As shown in FIG. 64A and FIG. 64B, the “2+1 IgG Crossfab” construct is the most potent molecule in this assay, followed by the “1+1 IgG Crossfab” and the “1+1 CrossMab”. This ranking is even more pronounced with MV-3 cells, expressing medium levels of MCSP, compared to high MCSP expressing WM266-4 cells. The calculated EC50 values on MV-3 cells were 9.2, 40.9 and 88.4 pM, on WM266-4 cells 33.1, 28.4 and 53.9 pM, for the “2+1 IgG Crossfab”, the “1+1 IgG Crossfab” and the “1+1 CrossMab”, respectively.

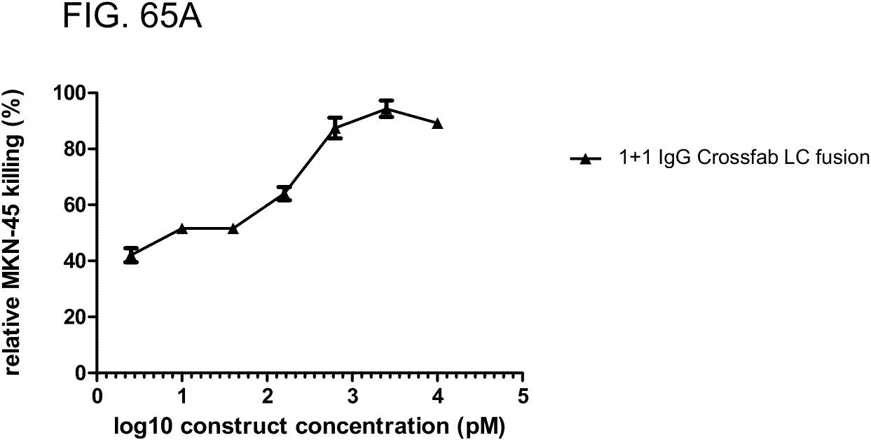

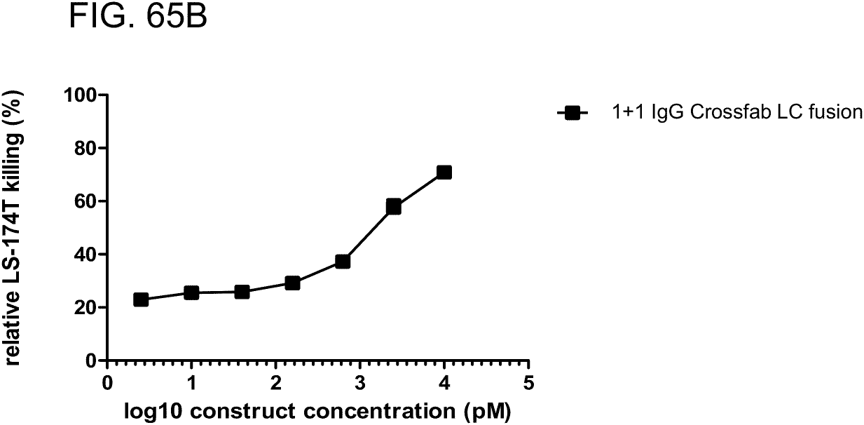

In a further experiment, different concentrations of the “1+1 IgG Crossfab LC fusion” construct (SEQ ID NOs 183, 209, 211, 213) were tested, using MKN-45 or LS-174T tumor target cells and human PBMC effector cells at an E:T ratio of 10:1 and an incubation time of 28 hours. As shown in FIG. 65A and FIG. 65B, the “1+1 IgG Crossfab LC fusion” construct induced apoptosis in MKN-45 target cells with a calculated EC50 of 213 pM, whereas the calculated EC50 is 1.56 nM with LS-174T cells, showing the influence of the different tumor antigen expression levels on the potency of the bispecific constructs within a certain period of time.

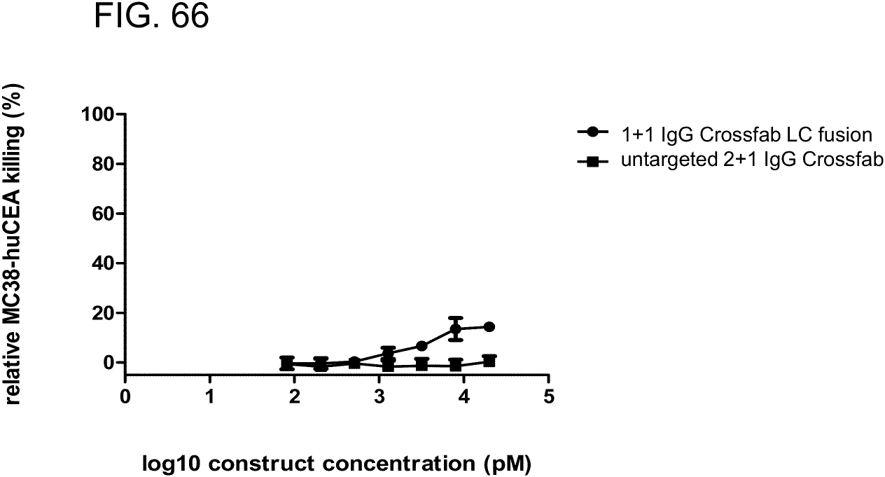

In yet another experiment, the “1+1 IgG Crossfab LC fusion” construct (SEQ ID NOs 183, 209, 211, 213) was compared to a untargeted “2+1 IgG Crossfab” molecule. MC38-huCEA tumor cells and human PBMCs (E:T ratio = 10:1) and an incubation time of 24 hours were used. As shown in FIG. 66, the “1+1 IgG Crossfab LC fusion” construct induced apoptosis of target cells in a concentration-dependent manner, with a calculated EC50 value of approximately 3.2 nM. In contrast, the untargeted “2+1 IgG Crossfab” showed antigen-independent T cell-mediated killing of target cells only at the highest concentration.

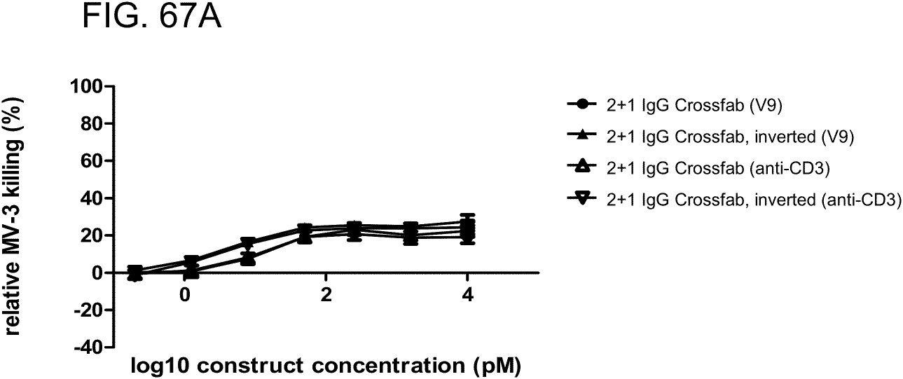

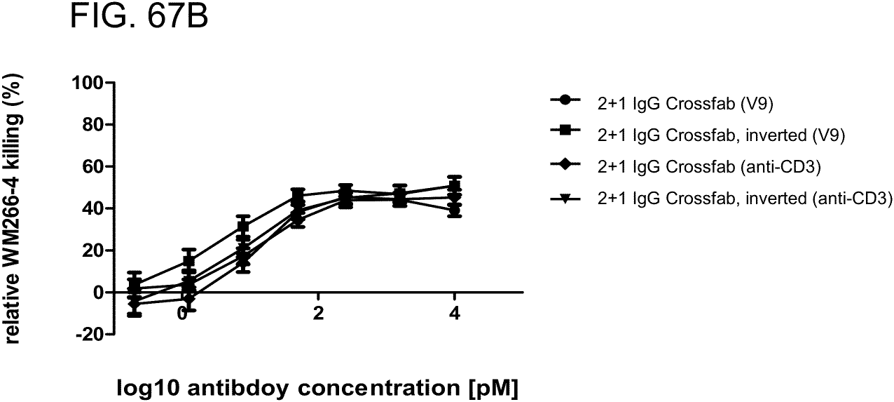

In a final experiment, the “2+1 IgG Crossfab (V9)” (SEQ ID NOs 3, 5, 29, 33), the “2+1 IgG Crossfab, inverted (V9)” (SEQ ID NOs 5, 23, 183, 187), the “2+1 IgG Crossfab (anti-CD3)” (SEQ ID NOs 5, 23, 215, 217), the “2+1 IgG Crossfab, inverted (anti-CD3)” (SEQ ID NOs 5, 23, 215, 219) were compared, using human MCSP-positive MV-3 or WM266-4 tumor cells and human PBMCs (E:T ratio = 10:1), and an incubation time of about 24 hours. As depicted in FIG. 67A and FIG. 67B, the T cell-mediated killing of the “2+1 IgG Crossfab, inverted” constructs seems to be slightly stronger or at least equal to the one induced by the “2+1 IgG Crossfabt” constructs for both CD3 binders. The calculated EC50 values were as follows:

| EC50 [pM] | 2+1 IgG Crossfab (V9) | 2+1 IgG Crossfab inverted (V9) | 2+1 IgG Crossfab (anti-CD3) | 2+1 IgG Crossfab, inverted (anti-CD3) |
|---|---|---|---|---|
| MV-3 | 10.0 | 4.1 | 11.0 | 3.0 |
| WM266-4 | 12.4 | 3.7 | 11.3 | 7.1 |

### Example 7 CD107a/b assay

Purified “2+1 IgG scFab” construct (SEQ ID NOs 5, 17, 19) and the “(scFv)₂” molecule, both targeting human MCSP and human CD3, were tested by flow cytometry for their potential to up-regulate CD107a and intracellular perforin levels in the presence or absence of human MCSP-expressing tumor cells.

Briefly, on day one, 30 000 Colo-38 tumor target cells per well were plated in a round-bottom 96-well plate and incubated overnight at 37° C., 5% CO₂ to let them adhere. Primary human pan T cells were isolated on day 1 or day 2 from Buffy Coat, as described.

On day two, 0.15 million effector cells per well were added to obtain a final E:T ratio of 5:1. FITC-conjugated CD107a/b antibodies, as well as the different bispecific constructs and controls are added. The different bispecific molecules and antibodies were adjusted to same molarities to obtain a final concentration of 9.43 nM. Following a 1 h incubation step at 37° C., 5% CO₂, monensin was added to inhibit secretion, but also to neutralize the pH within endosomes and lysosomes. After an additional incubation time of 5 h, cells were stained at 4° C. for 30 min for surface CD8 expression. Cells were washed with staining buffer (PBS / 0.1% BSA), fixed and permeabilized for 20 min using the BD Cytofix/Cytoperm Plus Kit with BD Golgi Stop (BD Biosciences #554715). Cells were washed twice using 1 × BD Perm/Wash buffer, and intracellular staining for perforin was performed at 4° C. for 30 min. After a final washing step with 1 × BD Perm/Wash buffer, cells were resuspended in PBS / 0.1% BSA and analyzed on FACS CantoII (all antibodies were purchased from BD Biosciences or BioLegend).

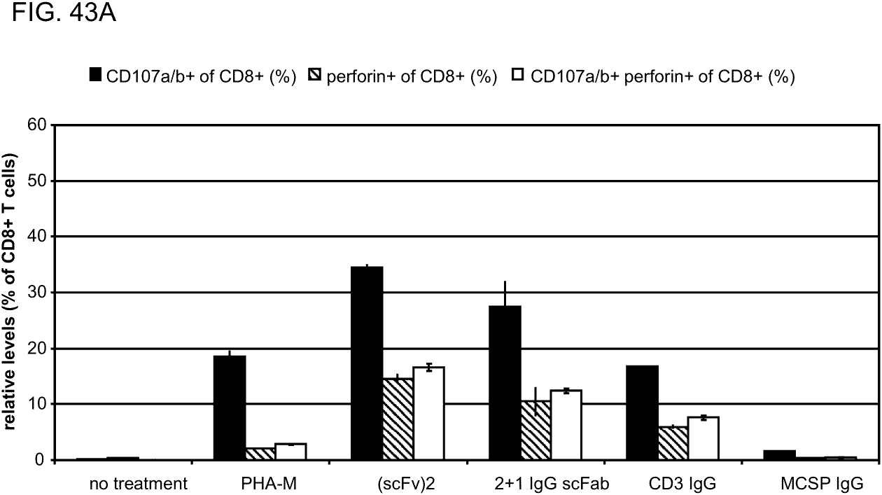

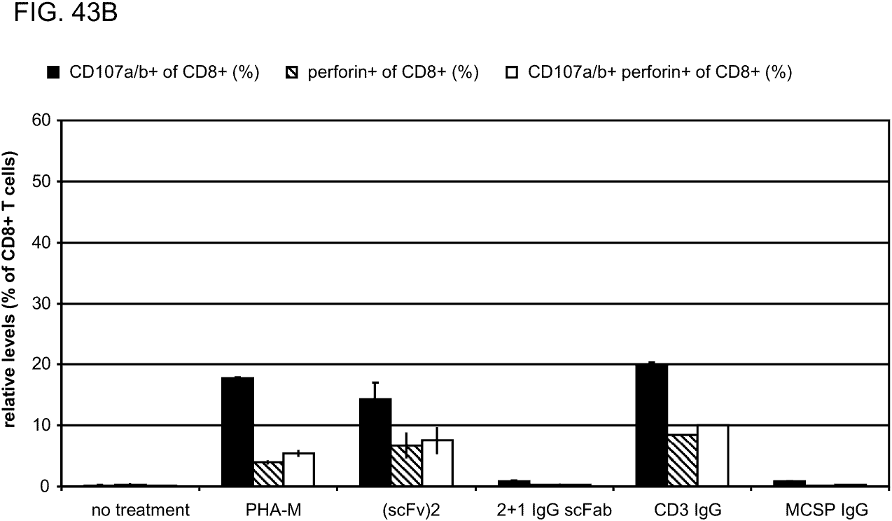

Gates were set either on all CD107a/b positive, perforin-positive or double-positive cells, as indicated (FIG. 43A and FIG. 43B). The “2+1 IgG scFab” construct was able to activate T cells and up-regulate CD107a/b and intracellular perforin levels only in the presence of target cells (FIG. 43A), whereas the “(scFv)₂” molecule shows (weak) induction of activation of T cells also in the absence of target cells (FIG. 43B). The bivalent reference anti-CD3 IgG results in a lower level of activation compared to the “(scFv)₂” molecule or the other bispecific construct.

### Example 8 Proliferation Assay

The purified “2+1 IgG scFab” (SEQ ID NOs 5, 17, 19) and “(scFv)₂” molecules, both targeting human CD3 and human MCSP, were tested by flow cytometry for their potential to induce proliferation of CD8⁺ or CD4⁺ T cells in the presence and absence of human MCSP-expressing tumor cells.

Briefly, freshly isolated human pan T cells were adjusted to 1 million cells per ml in warm PBS and stained with 1 µM CFSE at room temperature for 10 minutes. The staining volume was doubled by addition of RPMI1640 medium, containing 10% FCS and 1% GlutaMax. After incubation at room temperature for further 20 min, the cells were washed three times with pre-warmed medium to remove remaining CFSE. MCSP-positive Colo-38 cells were harvested with Cell Dissociation buffer, counted and checked for viability. Cells were adjusted to 0.2 × 10⁶ (viable) cells per ml in AIM-V medium, 100 µl of this cell suspension were pipetted per well into a round-bottom 96-well plate (as indicated). 50 µl of the (diluted) bispecific constructs were added to the cell-containing wells to obtain a final concentration of 1 nM. CFSE-stained human pan T effector cells were adjusted to 2 × 10⁶ (viable) cells per ml in AIM-V medium. 50 µl of this cell suspension was added per well of the assay plate (see above) to obtain a final E:T ratio of 5:1. To analyze whether the bispecific constructs are able to activate T cells only in the presence of target cells, expressing the tumor antigen huMCSP, wells were included that contained 1 nM of the respective bispecific molecules as well as PBMCs, but no target cells. After incubation for five days at 37° C., 5% CO₂, cells were centrifuged (5 min, 350 × g) and washed twice with 150 µl/well PBS, including 0.1% BSA. Surface staining for CD8 (mouse IgG1,κ; clone HIT8a; BD #555635), CD4 (mouse IgG1,κ; clone RPA-T4 ; BD #560649), or CD25 (mouse IgG1,κ; clone M-A251; BD #555434) was performed at 4° C. for 30 min, according to the supplier’s suggestions. Cells were washed twice with 150 µl/well PBS containing 0.1% BSA, resuspended in 200 µl/well PBS with 0.1% BSA, and analyzed using a FACS CantoII machine (Software FACS Diva). The relative proliferation level was determined by setting a gate around the non-proliferating cells and using the cell number of this gate relative to the overall measured cell number as the reference.

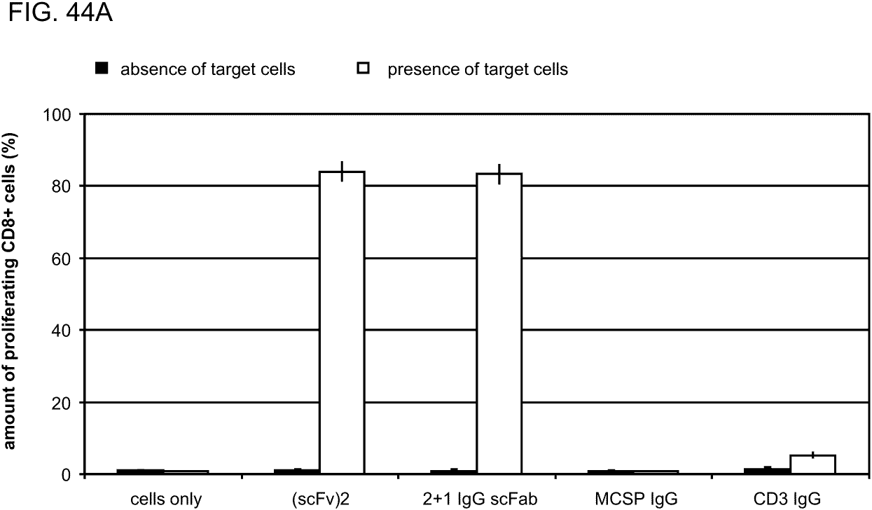

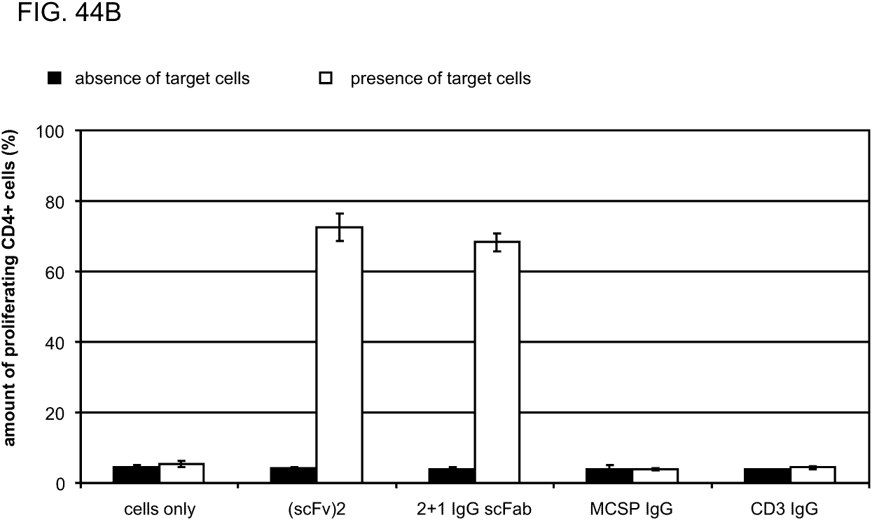

FIG. 44A and FIG. 44B shows that all constructs induce proliferation of CD8⁺ T cells (FIG. 44A) or CD4⁺ T cells (FIG. 44B) only in the presence of target cells, comparably to the “(scFv)₂” molecule. In general, activated CD8⁺ T cells proliferate more than activated CD4⁺ T cells in this assay.

### Example 9 Cytokine Release Assay

The purified “2+1 IgG scFab” construct (SEQ ID NOs 5, 17, 19) and the “(scFv)₂″molecule, both targeting human MCSP and human CD3, were analyzed for their ability to induce T cell-mediated de novo secretion of cytokines in the presence or absence of tumor target cells.

Briefly, human PBMCs were isolated from Buffy Coats and 0.3 million cells were plated per well into a round-bottom 96-well plate. Colo-38 tumor target cells, expressing human MCSP, were added to obtain a final E:T-ratio of 10:1. Bispecific constructs and IgG controls were added at 1 nM final concentration and the cells were incubated for 24 h at 37° C., 5% CO₂. The next day, the cells were centrifuged for 5 min at 350 × g and the supernatant was transferred into a new deep-well 96-well-plate for the subsequent analysis. The CBA analysis was performed according to manufacturer’s instructions for FACS CantoII, using the Human Th1/Th2 Cytokine Kit II (BD #551809).

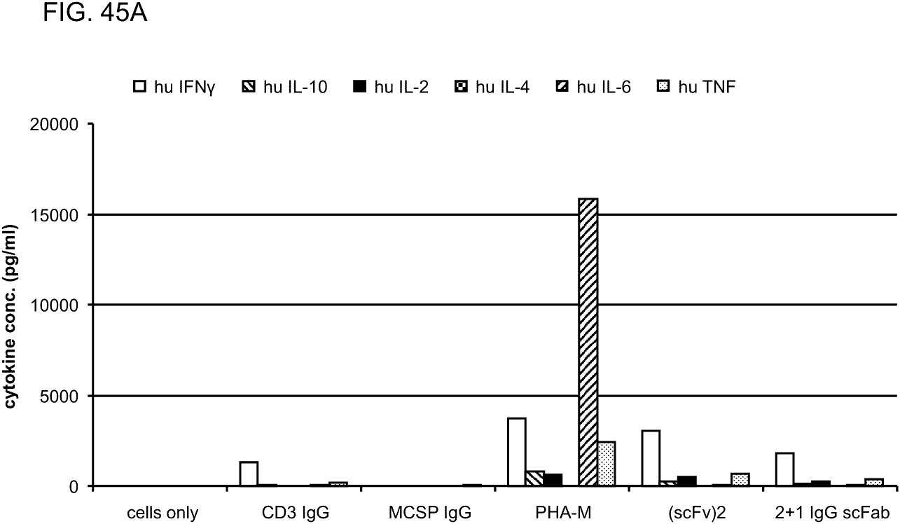

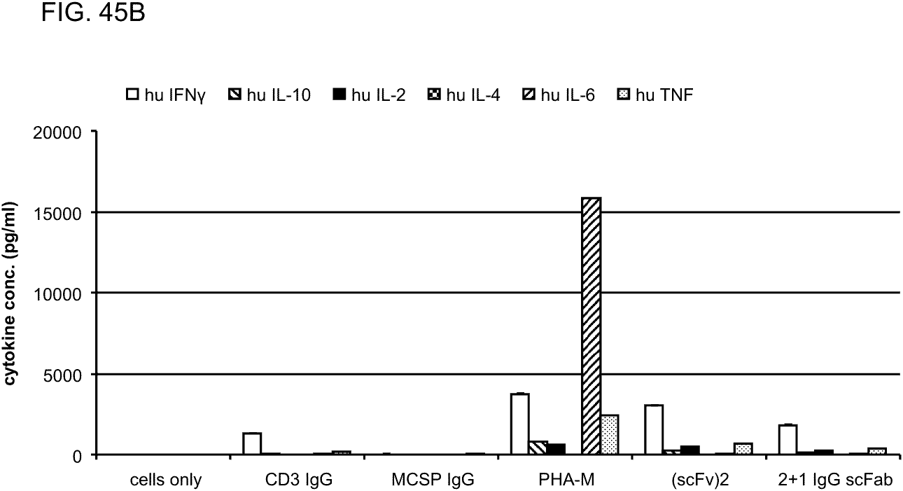

FIG. 45A and FIG. 45B show levels of the different cytokine measured in the supernatant. In the presence of target cells the main cytokine secreted upon T cell activation is IFN-y. The “(scFv)₂” molecule induces a slightly higher level of IFN-γ than the “2+1 IgG scFab” construct. The same tendency might be found for human TNF, but the overall levels of this cytokine were much lower compared to IFN-γ. There was no significant secretion of Th2 cytokines (IL-10 and IL-4) upon activation of T cells in the presence (or absence) of target cells. In the absence of Colo-38 target cells, only very weak induction of TNF secretion was observed, which was highest in samples treated with the “(scFv)₂” molecule.

In a second experiment, the following purified bispecific constructs targeting human MCSP and human CD3 were analyzed: the “2+1 IgG Crossfab” construct (SEQ ID NOs 3, 5, 29, 33), the “(scFv)₂” molecule, as well as different “2+1 IgG scFab” molecules comprising either a wild-type or a mutated (LALA, P329G and/or N297D, as indicated) Fc domain. Briefly, 280 µl whole blood from a healthy donor were plated per well of a deep-well 96-well plate. 30 000 Colo-38 tumor target cells, expressing human MCSP, as well as the different bispecific constructs and IgG controls were added at 1 nM final concentration. The cells were incubated for 24 h at 37° C., 5% CO₂ and then centrifuged for 5 min at 350 × g. The supernatant was transferred into a new deep-well 96-well-plate for the subsequent analysis. The CBA analysis was performed according to manufacturer’s instructions for FACS CantoII, using the combination of the following CBA Flex Sets: human granzyme B (BD #560304), human IFN-γ Flex Set (BD #558269), human TNF Flex Set (BD #558273), human IL-10 Flex Set (BD #558274), human IL-6 Flex Set (BD #558276), human IL-4 Flex Set (BD #558272), human IL-2 Flex Set (BD #558270).

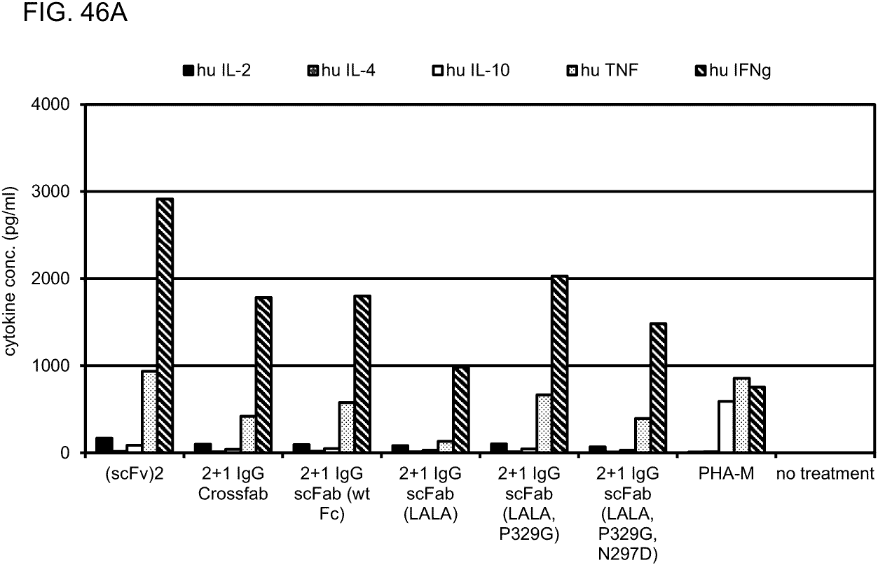

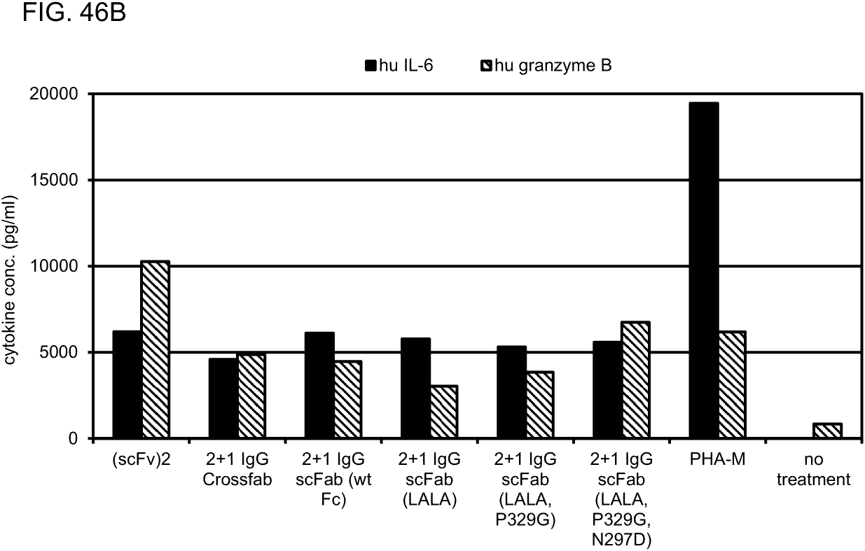

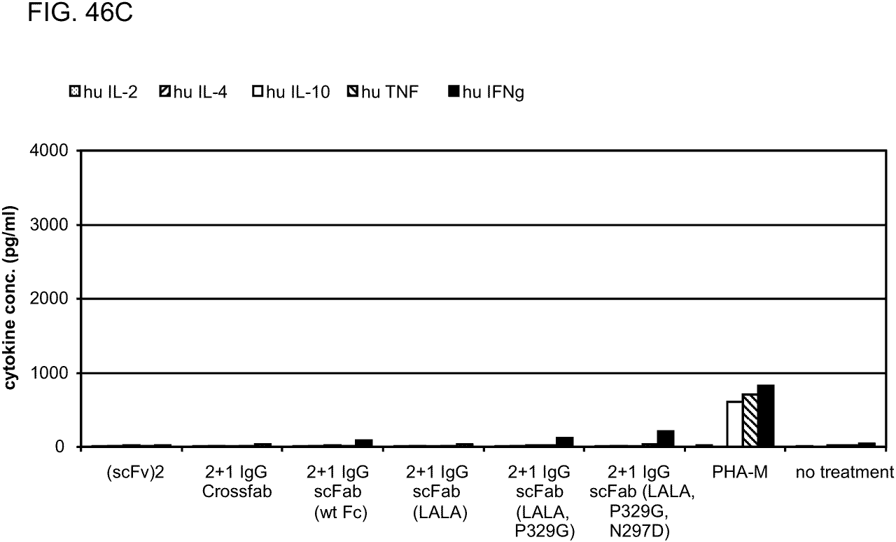

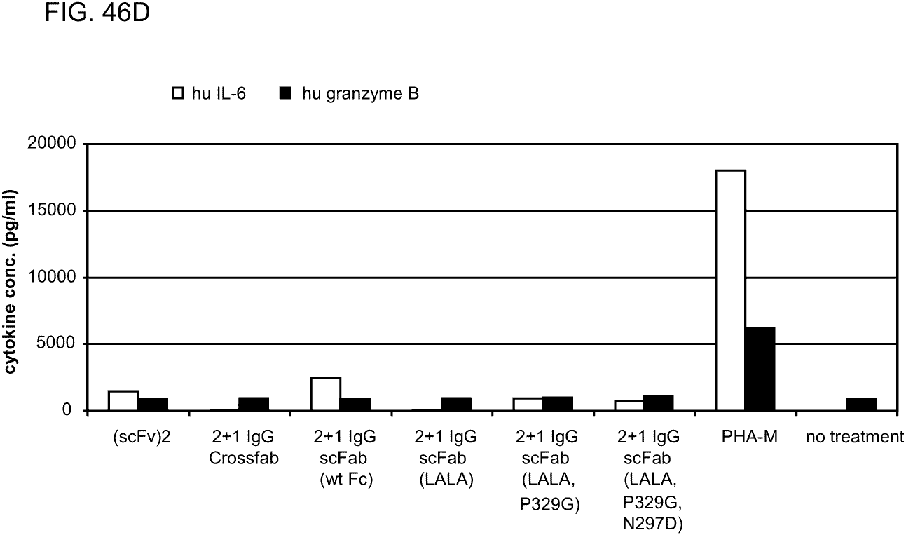

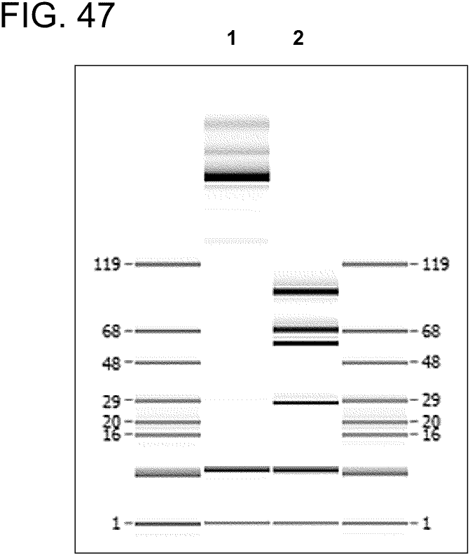

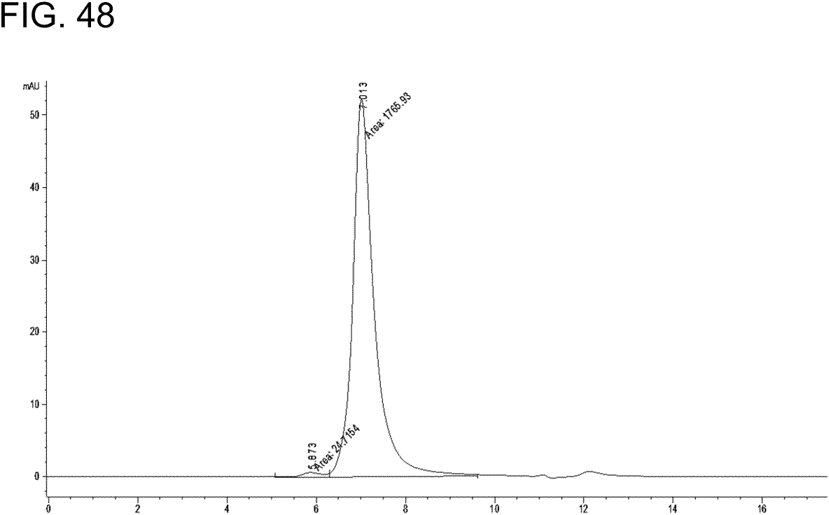

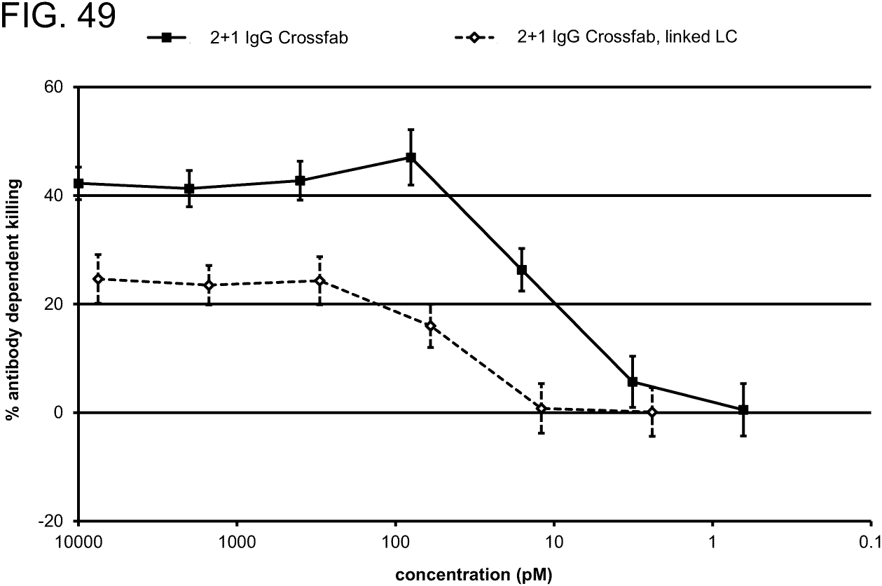

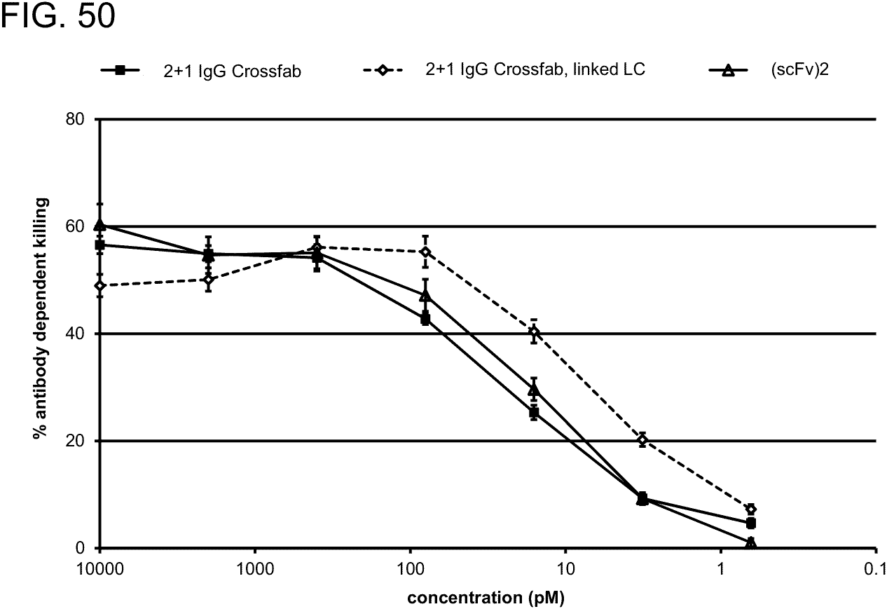

FIGS. 46A – 46D show the levels of the different cytokine measured in the supernatant. The main cytokine secreted in the presence of Colo-38 tumor cells was IL-6, followed by IFN-γ. In addition, also the levels of granzyme B strongly increased upon activation of T cells in the presence of target cells. In general, the “(scFv)₂” molecule induced higher levels of cytokine secretion in the presence of target cells (FIG. 46A and FIG. 46B). There was no significant secretion of Th2 cytokines (IL-10 and IL-4) upon activation of T cells in the presence (or absence) of target cells.

In this assay, there was a weak secretion of IFN-γ, induced by different “2+1 IgG scFab” constructs, even in the absence of target cells (FIG. 46C and FIG. 46D). Under these conditions, no significant differences could be observed between “2+1 IgG scFab” constructs with a wild-type or a mutated Fc domain.

### Listing of Sequences

266 total sequences generated using PatentIn version 3.5

SEQ ID NOs 1-148 and 150-264 are artificial sequences. SEQ ID NOs 149 and 265 are Homo sapien sequences. SEQ ID NO 266 is a Macaca fascicularis sequence.

SEQ ID NO: 1
<212 (molecule type)> PRT (Protein)
<223 (descriptor)> V9 (scFab)-Fc(hole) P329G LALA
Asp Ile Gln Met Thr Gln Ser Pro Ser Ser Leu Ser Ala Ser Val Gly
1 5 10 15
Asp Arg Val Thr Ile Thr Cys Arg Ala Ser Gln Asp Ile Arg Asn Tyr
20 25 30
Leu Asn Trp Tyr Gln Gln Lys Pro Gly Lys Ala Pro Lys Leu Leu Ile
35 40 45
Tyr Tyr Thr Ser Arg Leu Glu Ser Gly Val Pro Ser Arg Phe Ser Gly
50 55 60
Ser Gly Ser Gly Thr Asp Tyr Thr Leu Thr Ile Ser Ser Leu Gln Pro
65 70 75 80
Glu Asp Phe Ala Thr Tyr Tyr Cys Gln Gln Gly Asn Thr Leu Pro Trp
85 90 95
Thr Phe Gly Gln Gly Thr Lys Val Glu Ile Lys Arg Thr Val Ala Ala
100 105 110
Pro Ser Val Phe Ile Phe Pro Pro Ser Asp Glu Gln Leu Lys Ser Gly
115 120 125
Thr Ala Ser Val Val Cys Leu Leu Asn Asn Phe Tyr Pro Arg Glu Ala
130 135 140
Lys Val Gln Trp Lys Val Asp Asn Ala Leu Gln Ser Gly Asn Ser Gln
145 150 155 160
Glu Ser Val Thr Glu Gln Asp Ser Lys Asp Ser Thr Tyr Ser Leu Ser
165 170 175
Ser Thr Leu Thr Leu Ser Lys Ala Asp Tyr Glu Lys His Lys Val Tyr
180 185 190
Ala Cys Glu Val Thr His Gln Gly Leu Ser Ser Pro Val Thr Lys Ser
195 200 205
Phe Asn Arg Gly Glu Cys Ser Gly Gly Gly Ser Gly Gly Gly Ser Glu
210 215 220
Gly Gly Gly Ser Glu Gly Gly Gly Ser Glu Gly Gly Gly Ser Glu Gly
225 230 235 240
Gly Gly Ser Gly Gly Gly Ser Gly Glu Val Gln Leu Val Glu Ser Gly
245 250 255
Gly Gly Leu Val Gln Pro Gly Gly Ser Leu Arg Leu Ser Cys Ala Ala
260 265 270
Ser Gly Tyr Ser Phe Thr Gly Tyr Thr Met Asn Trp Val Arg Gln Ala
275 280 285
Pro Gly Lys Gly Leu Glu Trp Val Ala Leu Ile Asn Pro Tyr Lys Gly
290 295 300
Val Ser Thr Tyr Asn Gln Lys Phe Lys Asp Arg Phe Thr Ile Ser Val
305 310 315 320
Asp Lys Ser Lys Asn Thr Ala Tyr Leu Gln Met Asn Ser Leu Arg Ala
325 330 335
Glu Asp Thr Ala Val Tyr Tyr Cys Ala Arg Ser Gly Tyr Tyr Gly Asp
340 345 350
Ser Asp Trp Tyr Phe Asp Val Trp Gly Gln Gly Thr Leu Val Thr Val
355 360 365
Ser Ser Ala Ser Thr Lys Gly Pro Ser Val Phe Pro Leu Ala Pro Ser
370 375 380
Ser Lys Ser Thr Ser Gly Gly Thr Ala Ala Leu Gly Cys Leu Val Lys
385 390 395 400
Asp Tyr Phe Pro Glu Pro Val Thr Val Ser Trp Asn Ser Gly Ala Leu
405 410 415
Thr Ser Gly Val His Thr Phe Pro Ala Val Leu Gln Ser Ser Gly Leu
420 425 430
Tyr Ser Leu Ser Ser Val Val Thr Val Pro Ser Ser Ser Leu Gly Thr
435 440 445
Gln Thr Tyr Ile Cys Asn Val Asn His Lys Pro Ser Asn Thr Lys Val
450 455 460
Asp Lys Lys Val Glu Pro Lys Ser Cys Asp Lys Thr His Thr Cys Pro
465 470 475 480
Pro Cys Pro Ala Pro Glu Ala Ala Gly Gly Pro Ser Val Phe Leu Phe
485 490 495
Pro Pro Lys Pro Lys Asp Thr Leu Met Ile Ser Arg Thr Pro Glu Val
500 505 510
Thr Cys Val Val Val Asp Val Ser His Glu Asp Pro Glu Val Lys Phe
515 520 525
Asn Trp Tyr Val Asp Gly Val Glu Val His Asn Ala Lys Thr Lys Pro
530 535 540
Arg Glu Glu Gln Tyr Asn Ser Thr Tyr Arg Val Val Ser Val Leu Thr
545 550 555 560
Val Leu His Gln Asp Trp Leu Asn Gly Lys Glu Tyr Lys Cys Lys Val
565 570 575
Ser Asn Lys Ala Leu Gly Ala Pro Ile Glu Lys Thr Ile Ser Lys Ala
580 585 590
Lys Gly Gln Pro Arg Glu Pro Gln Val Cys Thr Leu Pro Pro Ser Arg
595 600 605
Asp Glu Leu Thr Lys Asn Gln Val Ser Leu Ser Cys Ala Val Lys Gly
610 615 620
Phe Tyr Pro Ser Asp Ile Ala Val Glu Trp Glu Ser Asn Gly Gln Pro
625 630 635 640
Glu Asn Asn Tyr Lys Thr Thr Pro Pro Val Leu Asp Ser Asp Gly Ser
645 650 655
Phe Phe Leu Val Ser Lys Leu Thr Val Asp Lys Ser Arg Trp Gln Gln
660 665 670
Gly Asn Val Phe Ser Cys Ser Val Met His Glu Ala Leu His Asn His
675 680 685
Tyr Thr Gln Lys Ser Leu Ser Leu Ser Pro Gly Lys
690 695 700

SEQ ID NO: 2
<212> DNA
<223> V9 (scFab)-Fc(hole) P329G LALA
gacatccaga tgacccagag cccctctagc ctgagcgcca gcgtgggcga cagagtgacc 60
atcacctgtc gggccagcca ggacatcaga aactacctga actggtatca gcagaagccc 120
ggcaaggccc ccaagctgct gatctactac acctctagac tggaaagcgg cgtgcccagc 180
cggtttagcg gcagcggctc cggcaccgac tacaccctga ccatcagcag cctgcagccc 240
gaggacttcg ccacctacta ctgccagcag ggcaacacac tcccctggac cttcggccag 300
ggcaccaagg tggagatcaa gcgtacggtg gccgctccca gcgtgttcat cttccccccc 360
agcgacgagc agctgaagtc cggcaccgcc agcgtcgtgt gcctgctgaa caacttctac 420
ccccgggagg ccaaggtgca gtggaaggtg gacaacgccc tgcagagcgg caacagccag 480
gaaagcgtca ccgagcagga cagcaaggac tccacctaca gcctgagcag caccctgacc 540
ctgagcaagg ccgactacga gaagcacaag gtgtacgcct gcgaagtgac ccaccagggc 600
ctgtccagcc ccgtgaccaa gagcttcaac cggggcgagt gcagcggcgg aggctctgga 660
ggcggctctg aaggcggagg aagtgagggc ggaggctcag aaggcggcgg aagcgaaggt 720
ggcggctctg gcggcggatc cggcgaggtg cagctggtcg agtccggcgg aggcctggtg 780
cagcctggcg gcagcctgag actgagctgc gccgccagcg gctacagctt caccggctac 840
accatgaact gggtccggca ggctcctggc aagggcctcg aatgggtggc cctgatcaac 900
ccctacaagg gcgtgagcac ctacaaccag aagttcaagg accggttcac catcagcgtg 960
gacaagagca agaacaccgc ctatctgcag atgaacagcc tgcgggccga ggacaccgcc 1020
gtgtactact gcgccagaag cggctactac ggcgacagcg actggtactt cgacgtgtgg 1080
ggccagggca cactggtcac cgtgtccagc gctagcacca agggcccatc ggtcttcccc 1140
ctggcaccct cctccaagag cacctctggg ggcacagcgg ccctgggctg cctggtcaag 1200
gactacttcc ccgaaccggt gacggtgtcg tggaactcag gcgccctgac cagcggcgtg 1260
cacaccttcc cggctgtcct acagtcctca ggactctact ccctcagcag cgtggtgacc 1320
gtgccctcca gcagcttggg cacccagacc tacatctgca acgtgaatca caagcccagc 1380
aacaccaagg tggacaagaa agttgagccc aaatcttgtg acaaaactca cacatgccca 1440
ccgtgcccag cacctgaagc tgcaggggga ccgtcagtct tcctcttccc cccaaaaccc 1500
aaggacaccc tcatgatctc ccggacccct gaggtcacat gcgtggtggt ggacgtgagc 1560
cacgaagacc ctgaggtcaa gttcaactgg tacgtggacg gcgtggaggt gcataatgcc 1620
aagacaaagc cgcgggagga gcagtacaac agcacgtacc gtgtggtcag cgtcctcacc 1680
gtcctgcacc aggactggct gaatggcaag gagtacaagt gcaaggtctc caacaaagcc 1740
ctcggcgccc ccatcgagaa aaccatctcc aaagccaaag ggcagccccg agaaccacag 1800
gtgtgcaccc tgcccccatc ccgggatgag ctgaccaaga accaggtcag cctctcgtgc 1860
gcagtcaaag gcttctatcc cagcgacatc gccgtggagt gggagagcaa tgggcagccg 1920
gagaacaact acaagaccac gcctcccgtg ctggactccg acggctcctt cttcctcgtg 1980
agcaagctca ccgtggacaa gagcaggtgg cagcagggga acgtcttctc atgctccgtg 2040
atgcatgagg ctctgcacaa ccactacacg cagaagagcc tctccctgtc tccgggtaaa 2100
tga 2103

SEQ ID NO: 3
<212> PRT
<223> LC007 (VH-CH1)-Fc(knob) P329G LALA
Glu Val Gln Leu Gln Glu Ser Gly Pro Gly Leu Val Lys Pro Ser Gln
1 5 10 15
Ser Leu Ser Leu Thr Cys Ser Val Thr Gly Tyr Ser Ile Thr Ser Gly
20 25 30
Tyr Tyr Trp Asn Trp Ile Arg Gln Phe Pro Gly Asn Lys Leu Glu Trp
35 40 45
Met Gly Tyr Ile Thr Tyr Asp Gly Ser Asn Asn Tyr Asn Pro Ser Leu
50 55 60
Lys Asn Arg Ile Ser Ile Thr Arg Asp Thr Ser Lys Asn Gln Phe Phe
65 70 75 80
Leu Lys Leu Asn Ser Val Thr Thr Glu Asp Thr Ala Thr Tyr Tyr Cys
85 90 95
Ala Asp Phe Asp Tyr Trp Gly Gln Gly Thr Thr Leu Thr Val Ser Ser
100 105 110
Ala Ser Thr Lys Gly Pro Ser Val Phe Pro Leu Ala Pro Ser Ser Lys
115 120 125
Ser Thr Ser Gly Gly Thr Ala Ala Leu Gly Cys Leu Val Lys Asp Tyr
130 135 140
Phe Pro Glu Pro Val Thr Val Ser Trp Asn Ser Gly Ala Leu Thr Ser
145 150 155 160
Gly Val His Thr Phe Pro Ala Val Leu Gln Ser Ser Gly Leu Tyr Ser
165 170 175
Leu Ser Ser Val Val Thr Val Pro Ser Ser Ser Leu Gly Thr Gln Thr
180 185 190
Tyr Ile Cys Asn Val Asn His Lys Pro Ser Asn Thr Lys Val Asp Lys
195 200 205
Lys Val Glu Pro Lys Ser Cys Asp Lys Thr His Thr Cys Pro Pro Cys
210 215 220
Pro Ala Pro Glu Ala Ala Gly Gly Pro Ser Val Phe Leu Phe Pro Pro
225 230 235 240
Lys Pro Lys Asp Thr Leu Met Ile Ser Arg Thr Pro Glu Val Thr Cys
245 250 255
Val Val Val Asp Val Ser His Glu Asp Pro Glu Val Lys Phe Asn Trp
260 265 270
Tyr Val Asp Gly Val Glu Val His Asn Ala Lys Thr Lys Pro Arg Glu
275 280 285
Glu Gln Tyr Asn Ser Thr Tyr Arg Val Val Ser Val Leu Thr Val Leu
290 295 300
His Gln Asp Trp Leu Asn Gly Lys Glu Tyr Lys Cys Lys Val Ser Asn
305 310 315 320
Lys Ala Leu Gly Ala Pro Ile Glu Lys Thr Ile Ser Lys Ala Lys Gly
325 330 335
Gln Pro Arg Glu Pro Gln Val Tyr Thr Leu Pro Pro Cys Arg Asp Glu
340 345 350
Leu Thr Lys Asn Gln Val Ser Leu Trp Cys Leu Val Lys Gly Phe Tyr
355 360 365
Pro Ser Asp Ile Ala Val Glu Trp Glu Ser Asn Gly Gln Pro Glu Asn
370 375 380
Asn Tyr Lys Thr Thr Pro Pro Val Leu Asp Ser Asp Gly Ser Phe Phe
385 390 395 400
Leu Tyr Ser Lys Leu Thr Val Asp Lys Ser Arg Trp Gln Gln Gly Asn
405 410 415
Val Phe Ser Cys Ser Val Met His Glu Ala Leu His Asn His Tyr Thr
420 425 430
Gln Lys Ser Leu Ser Leu Ser Pro Gly Lys
435 440

SEQ ID NO: 4
<212> DNA
<223> LC007 (VH-CH1)-Fc(knob) P329G LALA
gaggtccagc tgcaggagtc aggacctggc ctcgtgaaac cttctcagtc tctgtctctc 60
acctgctctg tcactggcta ctccatcacc agtggttatt actggaactg gatccggcag 120
tttccaggaa acaagctgga atggatgggc tacataacct acgacggtag caataactac 180
aacccatctc tcaaaaatcg aatctccatc actcgtgaca catctaagaa ccagtttttc 240
ctgaagttga attctgtgac tactgaggac acagctacat attactgtgc ggactttgac 300
tactggggcc aaggcaccac tctcacagtc tcctcagcta gcaccaaggg cccatcggtc 360
ttccccctgg caccctcctc caagagcacc tctgggggca cagcggccct gggctgcctg 420
gtcaaggact acttccccga accggtgacg gtgtcgtgga actcaggcgc cctgaccagc 480
ggcgtgcaca ccttcccggc tgtcctacag tcctcaggac tctactccct cagcagcgtg 540
gtgaccgtgc cctccagcag cttgggcacc cagacctaca tctgcaacgt gaatcacaag 600
cccagcaaca ccaaggtgga caagaaagtt gagcccaaat cttgtgacaa gacccacacc 660
tgtccccctt gccctgcccc tgaagctgct ggtggccctt ccgtgttcct gttcccccca 720
aagcccaagg acaccctgat gatcagccgg acccccgaag tgacctgcgt ggtggtcgat 780
gtgtcccacg aggaccctga agtgaagttc aattggtacg tggacggcgt ggaagtgcac 840
aatgccaaga ccaagccgcg ggaggagcag tacaacagca cgtaccgtgt ggtcagcgtc 900
ctcaccgtcc tgcaccagga ctggctgaat ggcaaggagt acaagtgcaa ggtctccaac 960
aaagccctcg gcgcccccat cgagaaaacc atctccaaag ccaaagggca gccccgagaa 1020
ccacaggtgt acaccctgcc cccatgccgg gatgagctga ccaagaacca ggtcagcctg 1080
tggtgcctgg tcaaaggctt ctatcccagc gacatcgccg tggagtggga gagcaatggg 1140
cagccggaga acaactacaa gaccacgcct cccgtgctgg actccgacgg ctccttcttc 1200
ctctacagca agctcaccgt ggacaagagc aggtggcagc aggggaacgt cttctcatgc 1260
tccgtgatgc atgaggctct gcacaaccac tacacgcaga agagcctctc cctgtctccg 1320
ggtaaataa 1329

SEQ ID NO: 5
<212> PRT
<223> LC007 (VL-CL)
Asp Ile Val Leu Thr Gln Ser Pro Ser Ser Leu Ser Ala Ser Leu Gly
1 5 10 15
Asp Arg Val Thr Ile Ser Cys Ser Ala Ser Gln Gly Ile Arg Asn Tyr
20 25 30
Leu Asn Trp Tyr Gln Gln Arg Pro Asp Gly Thr Val Lys Leu Leu Ile
35 40 45
Tyr Tyr Thr Ser Ser Leu His Ser Gly Val Pro Ser Arg Phe Ser Gly
50 55 60
Ser Gly Ser Gly Thr Asp Tyr Ser Leu Thr Ile Ser Asn Leu Glu Pro
65 70 75 80
Glu Asp Ile Ala Thr Tyr Tyr Cys Gln Gln Tyr Ser Lys Leu Pro Trp
85 90 95
Thr Phe Gly Gly Gly Thr Lys Leu Glu Ile Lys Arg Thr Val Ala Ala
100 105 110
Pro Ser Val Phe Ile Phe Pro Pro Ser Asp Glu Gln Leu Lys Ser Gly
115 120 125
Thr Ala Ser Val Val Cys Leu Leu Asn Asn Phe Tyr Pro Arg Glu Ala
130 135 140
Lys Val Gln Trp Lys Val Asp Asn Ala Leu Gln Ser Gly Asn Ser Gln
145 150 155 160
Glu Ser Val Thr Glu Gln Asp Ser Lys Asp Ser Thr Tyr Ser Leu Ser
165 170 175
Ser Thr Leu Thr Leu Ser Lys Ala Asp Tyr Glu Lys His Lys Val Tyr
180 185 190
Ala Cys Glu Val Thr His Gln Gly Leu Ser Ser Pro Val Thr Lys Ser
195 200 205
Phe Asn Arg Gly Glu Cys
210

SEQ ID NO: 6
<212> DNA
<223> LC007 (VL-CL)
gatattgtgc tcacacagtc tccatcctcc ctgtctgcct ctctgggaga cagagtcacc 60
atcagttgca gtgcaagtca gggcattaga aattatttaa actggtatca gcagagacca 120
gatggaactg ttaaactcct gatctattac acatcaagtt tacactcagg agtcccatca 180
aggttcagtg gcagtgggtc tgggacagat tattctctca ccatcagcaa cctggaacct 240
gaagatattg ccacttacta ttgtcagcag tatagtaagc ttccttggac gttcggtgga 300
ggcaccaagc tggaaatcaa acgtacggtg gctgcaccat ctgtcttcat cttcccgcca 360
tctgatgagc agttgaaatc tggaactgcc tctgttgtgt gcctgctgaa taacttctat 420
cccagagagg ccaaagtaca gtggaaggtg gataacgccc tccaatcggg taactcccag 480
gagagtgtca cagagcagga cagcaaggac agcacctaca gcctcagcag caccctgacg 540
ctgagcaaag cagactacga gaaacacaaa gtctacgcct gcgaagtcac ccatcagggc 600
ctgagctcgc ccgtcacaaa gagcttcaac aggggagagt gttag 645

SEQ ID NO: 7
<212> PRT
<223> LC007 (scFab)-Fc(hole) P329G LALA
Asp Ile Val Leu Thr Gln Ser Pro Ser Ser Leu Ser Ala Ser Leu Gly
1 5 10 15
Asp Arg Val Thr Ile Ser Cys Ser Ala Ser Gln Gly Ile Arg Asn Tyr
20 25 30
Leu Asn Trp Tyr Gln Gln Arg Pro Asp Gly Thr Val Lys Leu Leu Ile
35 40 45
Tyr Tyr Thr Ser Ser Leu His Ser Gly Val Pro Ser Arg Phe Ser Gly
50 55 60
Ser Gly Ser Gly Thr Asp Tyr Ser Leu Thr Ile Ser Asn Leu Glu Pro
65 70 75 80
Glu Asp Ile Ala Thr Tyr Tyr Cys Gln Gln Tyr Ser Lys Leu Pro Trp
85 90 95
Thr Phe Gly Gly Gly Thr Lys Leu Glu Ile Lys Arg Thr Val Ala Ala
100 105 110
Pro Ser Val Phe Ile Phe Pro Pro Ser Asp Glu Gln Leu Lys Ser Gly
115 120 125
Thr Ala Ser Val Val Cys Leu Leu Asn Asn Phe Tyr Pro Arg Glu Ala
130 135 140
Lys Val Gln Trp Lys Val Asp Asn Ala Leu Gln Ser Gly Asn Ser Gln
145 150 155 160
Glu Ser Val Thr Glu Gln Asp Ser Lys Asp Ser Thr Tyr Ser Leu Ser
165 170 175
Ser Thr Leu Thr Leu Ser Lys Ala Asp Tyr Glu Lys His Lys Val Tyr
180 185 190
Ala Cys Glu Val Thr His Gln Gly Leu Ser Ser Pro Val Thr Lys Ser
195 200 205
Phe Asn Arg Gly Glu Cys Ser Gly Gly Gly Ser Gly Gly Gly Ser Glu
210 215 220
Gly Gly Gly Ser Glu Gly Gly Gly Ser Glu Gly Gly Gly Ser Glu Gly
225 230 235 240
Gly Gly Ser Gly Gly Gly Ser Gly Glu Val Gln Leu Gln Glu Ser Gly
245 250 255
Pro Gly Leu Val Lys Pro Ser Gln Ser Leu Ser Leu Thr Cys Ser Val
260 265 270
Thr Gly Tyr Ser Ile Thr Ser Gly Tyr Tyr Trp Asn Trp Ile Arg Gln
275 280 285
Phe Pro Gly Asn Lys Leu Glu Trp Met Gly Tyr Ile Thr Tyr Asp Gly
290 295 300
Ser Asn Asn Tyr Asn Pro Ser Leu Lys Asn Arg Ile Ser Ile Thr Arg
305 310 315 320
Asp Thr Ser Lys Asn Gln Phe Phe Leu Lys Leu Asn Ser Val Thr Thr
325 330 335
Glu Asp Thr Ala Thr Tyr Tyr Cys Ala Asp Phe Asp Tyr Trp Gly Gln
340 345 350
Gly Thr Thr Leu Thr Val Ser Ser Ala Ser Thr Lys Gly Pro Ser Val
355 360 365
Phe Pro Leu Ala Pro Ser Ser Lys Ser Thr Ser Gly Gly Thr Ala Ala
370 375 380
Leu Gly Cys Leu Val Lys Asp Tyr Phe Pro Glu Pro Val Thr Val Ser
385 390 395 400
Trp Asn Ser Gly Ala Leu Thr Ser Gly Val His Thr Phe Pro Ala Val
405 410 415
Leu Gln Ser Ser Gly Leu Tyr Ser Leu Ser Ser Val Val Thr Val Pro
420 425 430
Ser Ser Ser Leu Gly Thr Gln Thr Tyr Ile Cys Asn Val Asn His Lys
435 440 445
Pro Ser Asn Thr Lys Val Asp Lys Lys Val Glu Pro Lys Ser Cys Asp
450 455 460
Gly Gly Gly Gly Ser Gly Gly Gly Gly Ser Gly Ala Gln Asp Lys Thr
465 470 475 480
His Thr Cys Pro Pro Cys Pro Ala Pro Glu Ala Ala Gly Gly Pro Ser
485 490 495
Val Phe Leu Phe Pro Pro Lys Pro Lys Asp Thr Leu Met Ile Ser Arg
500 505 510
Thr Pro Glu Val Thr Cys Val Val Val Asp Val Ser His Glu Asp Pro
515 520 525
Glu Val Lys Phe Asn Trp Tyr Val Asp Gly Val Glu Val His Asn Ala
530 535 540
Lys Thr Lys Pro Arg Glu Glu Gln Tyr Asn Ser Thr Tyr Arg Val Val
545 550 555 560
Ser Val Leu Thr Val Leu His Gln Asp Trp Leu Asn Gly Lys Glu Tyr
565 570 575
Lys Cys Lys Val Ser Asn Lys Ala Leu Pro Ala Pro Ile Glu Lys Thr
580 585 590
Ile Ser Lys Ala Lys Gly Gln Pro Arg Glu Pro Gln Val Cys Thr Leu
595 600 605
Pro Pro Ser Arg Asp Glu Leu Thr Lys Asn Gln Val Ser Leu Ser Cys
610 615 620
Ala Val Lys Gly Phe Tyr Pro Ser Asp Ile Ala Val Glu Trp Glu Ser
625 630 635 640
Asn Gly Gln Pro Glu Asn Asn Tyr Lys Thr Thr Pro Pro Val Leu Asp
645 650 655
Ser Asp Gly Ser Phe Phe Leu Val Ser Lys Leu Thr Val Asp Lys Ser
660 665 670
Arg Trp Gln Gln Gly Asn Val Phe Ser Cys Ser Val Met His Glu Ala
675 680 685
Leu His Asn His Tyr Thr Gln Lys Ser Leu Ser Leu Ser Pro Gly Lys
690 695 700

SEQ ID NO: 8
<212> DNA
<223> LC007 (scFab)-Fc(hole) P329G LALA
gacatcgtgc tgacccagag ccctagcagc ctgagcgcca gcctgggcga cagagtgacc 60
atcagctgta gcgcctccca gggcatcaga aactacctga actggtatca gcagagaccc 120
gacggcacag tgaagctgct gatctactac accagcagcc tgcacagcgg cgtgccaagc 180
agattcagcg gcagcggctc cggcacagac tacagcctga ccatctccaa cctggaaccc 240
gaggatatcg ccacctacta ctgccagcag tacagcaagc tgccctggac cttcggcgga 300
ggcaccaagc tggaaatcaa gcggaccgtg gccgctccca gcgtgttcat cttcccaccc 360
agcgacgagc agctgaagtc cggcacagcc agcgtcgtgt gcctgctgaa caacttctac 420
ccccgggagg ccaaggtgca gtggaaggtg gacaacgccc tgcagagcgg caacagccag 480
gaaagcgtca ccgagcagga cagcaaggac tccacctaca gcctgtccag caccctgacc 540
ctgagcaagg ccgactacga gaagcacaag gtgtacgcct gcgaagtgac ccaccagggc 600
ctgagcagcc ccgtgaccaa gagcttcaac cggggcgagt gtagtggcgg aggctctggc 660
ggaggaagcg agggcggagg atctgaaggc ggcggatctg aggggggagg cagtgaaggg 720
ggaggctcag ggggaggatc cggcgaggtg cagctgcagg aatctggccc tggcctggtc 780
aagccaagcc agagtctgag cctgacctgc agcgtgaccg gctacagcat taccagcggc 840
tactactgga actggattcg gcagttcccc ggcaataagc tggaatggat gggctacatc 900
acctacgacg gcagcaacaa ctacaacccc agcctgaaga accggatcag catcacccgg 960
gacaccagca agaaccagtt cttcctgaag ctgaacagcg tgaccaccga ggacaccgcc 1020
acatactatt gcgccgactt cgactactgg ggccagggca ccaccctgac cgtgtccagc 1080
gccagcacaa agggccctag cgtgttccct ctggccccca gcagcaagag cacaagcggc 1140
ggaacagccg ccctgggctg cctcgtgaag gactacttcc ccgagcccgt gacagtgtct 1200
tggaacagcg gagccctgac aagcggcgtg cacaccttcc ctgccgtgct gcagagcagc 1260
ggcctgtact ccctgagcag cgtggtcacc gtgcctagca gcagcctggg cacccagacc 1320
tacatctgca acgtgaacca caagcccagc aacaccaaag tggacaagaa ggtggagccc 1380
aagagctgtg atggcggagg agggtccgga ggcggtggat ccggagctca ggacaaaact 1440
cacacatgcc caccgtgccc agcacctgaa gctgcagggg gaccgtcagt cttcctcttc 1500
cccccaaaac ccaaggacac cctcatgatc tcccggaccc ctgaggtcac atgcgtggtg 1560
gtggacgtga gccacgaaga ccctgaggtc aagttcaact ggtacgtgga cggcgtggag 1620
gtgcataatg ccaagacaaa gccgcgggag gagcagtaca acagcacgta ccgtgtggtc 1680
agcgtcctca ccgtcctgca ccaggactgg ctgaatggca aggagtacaa gtgcaaggtc 1740
tccaacaaag ccctcccagc ccccatcgag aaaaccatct ccaaagccaa agggcagccc 1800
cgagaaccac aggtgtgcac cctgccccca tcccgggatg agctgaccaa gaaccaggtc 1860
agcctctcgt gcgcagtcaa aggcttctat cccagcgaca tcgccgtgga gtgggagagc 1920
aatgggcagc cggagaacaa ctacaagacc acgcctcccg tgctggactc cgacggctcc 1980
ttcttcctcg tgagcaagct caccgtggac aagagcaggt ggcagcaggg gaacgtcttc 2040
tcatgctccg tgatgcatga ggctctgcac aaccactaca cgcagaagag cctctccctg 2100
tctccgggta aatga 2115

SEQ ID NO: 9
<212> PRT
<223> V9 (VH-CH1) -Fc(knob) LALA
Glu Val Gln Leu Val Glu Ser Gly Gly Gly Leu Val Gln Pro Gly Gly
1 5 10 15
Ser Leu Arg Leu Ser Cys Ala Ala Ser Gly Tyr Ser Phe Thr Gly Tyr
20 25 30
Thr Met Asn Trp Val Arg Gln Ala Pro Gly Lys Gly Leu Glu Trp Val
35 40 45
Ala Leu Ile Asn Pro Tyr Lys Gly Val Ser Thr Tyr Asn Gln Lys Phe
50 55 60
Lys Asp Arg Phe Thr Ile Ser Val Asp Lys Ser Lys Asn Thr Ala Tyr
65 70 75 80
Leu Gln Met Asn Ser Leu Arg Ala Glu Asp Thr Ala Val Tyr Tyr Cys
85 90 95
Ala Arg Ser Gly Tyr Tyr Gly Asp Ser Asp Trp Tyr Phe Asp Val Trp
100 105 110
Gly Gln Gly Thr Leu Val Thr Val Ser Ser Ala Ser Thr Lys Gly Pro
115 120 125
Ser Val Phe Pro Leu Ala Pro Ser Ser Lys Ser Thr Ser Gly Gly Thr
130 135 140
Ala Ala Leu Gly Cys Leu Val Lys Asp Tyr Phe Pro Glu Pro Val Thr
145 150 155 160
Val Ser Trp Asn Ser Gly Ala Leu Thr Ser Gly Val His Thr Phe Pro
165 170 175
Ala Val Leu Gln Ser Ser Gly Leu Tyr Ser Leu Ser Ser Val Val Thr
180 185 190
Val Pro Ser Ser Ser Leu Gly Thr Gln Thr Tyr Ile Cys Asn Val Asn
195 200 205
His Lys Pro Ser Asn Thr Lys Val Asp Lys Lys Val Glu Pro Lys Ser
210 215 220
Cys Asp Gly Gly Gly Gly Ser Gly Gly Gly Gly Ser Gly Ala Gln Asp
225 230 235 240
Lys Thr His Thr Cys Pro Pro Cys Pro Ala Pro Glu Ala Ala Gly Gly
245 250 255
Pro Ser Val Phe Leu Phe Pro Pro Lys Pro Lys Asp Thr Leu Met Ile
260 265 270
Ser Arg Thr Pro Glu Val Thr Cys Val Val Val Asp Val Ser His Glu
275 280 285
Asp Pro Glu Val Lys Phe Asn Trp Tyr Val Asp Gly Val Glu Val His
290 295 300
Asn Ala Lys Thr Lys Pro Arg Glu Glu Gln Tyr Asn Ser Thr Tyr Arg
305 310 315 320
Val Val Ser Val Leu Thr Val Leu His Gln Asp Trp Leu Asn Gly Lys
325 330 335
Glu Tyr Lys Cys Lys Val Ser Asn Lys Ala Leu Pro Ala Pro Ile Glu
340 345 350
Lys Thr Ile Ser Lys Ala Lys Gly Gln Pro Arg Glu Pro Gln Val Tyr
355 360 365
Thr Leu Pro Pro Cys Arg Asp Glu Leu Thr Lys Asn Gln Val Ser Leu
370 375 380
Trp Cys Leu Val Lys Gly Phe Tyr Pro Ser Asp Ile Ala Val Glu Trp
385 390 395 400
Glu Ser Asn Gly Gln Pro Glu Asn Asn Tyr Lys Thr Thr Pro Pro Val
405 410 415
Leu Asp Ser Asp Gly Ser Phe Phe Leu Tyr Ser Lys Leu Thr Val Asp
420 425 430
Lys Ser Arg Trp Gln Gln Gly Asn Val Phe Ser Cys Ser Val Met His
435 440 445
Glu Ala Leu His Asn His Tyr Thr Gln Lys Ser Leu Ser Leu Ser Pro
450 455 460
Gly Lys
465

SEQ ID NO: 10
<212> DNA
<223> V9 (VH-CH1) -Fc(knob) LALA
gaggtgcagc tggtcgagag cggaggcggc ctggtgcagc ctggcggcag cctgagactg 60
agctgcgccg ccagcggcta cagcttcacc ggctacacca tgaactgggt ccggcaggca 120
cctggcaagg gactggaatg ggtggccctg atcaacccct acaagggcgt gagcacctac 180
aaccagaagt tcaaggaccg gttcaccatc agcgtggaca agagcaagaa caccgcctat 240
ctgcagatga acagcctgcg ggccgaggac accgccgtgt actactgcgc cagaagcggc 300
tactacggcg acagcgactg gtacttcgac gtgtggggcc agggcaccct cgtgaccgtg 360
tctagcgcta gcaccaaggg cccctccgtg ttccccctgg cccccagcag caagagcacc 420
agcggcggca cagccgctct gggctgcctg gtcaaggact acttccccga gcccgtgacc 480
gtgtcctgga acagcggagc cctgacctcc ggcgtgcaca ccttccccgc cgtgctgcag 540
agttctggcc tgtatagcct gagcagcgtg gtcaccgtgc cttctagcag cctgggcacc 600
cagacctaca tctgcaacgt gaaccacaag cccagcaaca ccaaggtgga caagaaggtg 660
gagcccaaga gctgcgacgg cggtggtggc tccggaggcg gtggatccgg agctcaggac 720
aaaactcaca catgcccacc gtgcccagca cctgaagctg cagggggacc gtcagtcttc 780
ctcttccccc caaaacccaa ggacaccctc atgatctccc ggacccctga ggtcacatgc 840
gtggtggtgg acgtgagcca cgaagaccct gaggtcaagt tcaactggta cgtggacggc 900
gtggaggtgc ataatgccaa gacaaagccg cgggaggagc agtacaacag cacgtaccgt 960
gtggtcagcg tcctcaccgt cctgcaccag gactggctga atggcaagga gtacaagtgc 1020
aaggtctcca acaaagccct cccagccccc atcgagaaaa ccatctccaa agccaaaggg 1080
cagccccgag aaccacaggt gtacaccctg cccccatgcc gggatgagct gaccaagaac 1140
caggtcagcc tgtggtgcct ggtcaaaggc ttctatccca gcgacatcgc cgtggagtgg 1200
gagagcaatg ggcagccgga gaacaactac aagaccacgc ctcccgtgct ggactccgac 1260
ggctccttct tcctctacag caagctcacc gtggacaaga gcaggtggca gcaggggaac 1320
gtcttctcat gctccgtgat gcatgaggct ctgcacaacc actacacgca gaagagcctc 1380
tccctgtctc cgggtaaatg a 1401

SEQ ID NO: 11
<212> PRT
<223> V9 (VL-CL)
Asp Ile Gln Met Thr Gln Ser Pro Ser Ser Leu Ser Ala Ser Val Gly
1 5 10 15
Asp Arg Val Thr Ile Thr Cys Arg Ala Ser Gln Asp Ile Arg Asn Tyr
20 25 30
Leu Asn Trp Tyr Gln Gln Lys Pro Gly Lys Ala Pro Lys Leu Leu Ile
35 40 45
Tyr Tyr Thr Ser Arg Leu Glu Ser Gly Val Pro Ser Arg Phe Ser Gly
50 55 60
Ser Gly Ser Gly Thr Asp Tyr Thr Leu Thr Ile Ser Ser Leu Gln Pro
65 70 75 80
Glu Asp Phe Ala Thr Tyr Tyr Cys Gln Gln Gly Asn Thr Leu Pro Trp
85 90 95
Thr Phe Gly Gln Gly Thr Lys Val Glu Ile Lys Arg Thr Val Ala Ala
100 105 110
Pro Ser Val Phe Ile Phe Pro Pro Ser Asp Glu Gln Leu Lys Ser Gly
115 120 125
Thr Ala Ser Val Val Cys Leu Leu Asn Asn Phe Tyr Pro Arg Glu Ala
130 135 140
Lys Val Gln Trp Lys Val Asp Asn Ala Leu Gln Ser Gly Asn Ser Gln
145 150 155 160
Glu Ser Val Thr Glu Gln Asp Ser Lys Asp Ser Thr Tyr Ser Leu Ser
165 170 175
Ser Thr Leu Thr Leu Ser Lys Ala Asp Tyr Glu Lys His Lys Val Tyr
180 185 190
Ala Cys Glu Val Thr His Gln Gly Leu Ser Ser Pro Val Thr Lys Ser
195 200 205
Phe Asn Arg Gly Glu Cys
210

SEQ ID NO: 12
<212> DNA
<223> V9 (VL-CL)
gacatccaga tgacccagag ccccagcagc ctgagcgcca gcgtgggcga cagagtgacc 60
atcacctgtc gggccagcca ggacatcaga aactacctga actggtatca gcagaagccc 120
ggcaaggccc ccaagctgct gatctactac acctctagac tggaaagcgg cgtgcccagc 180
cggtttagcg gcagcggctc cggcaccgac tacaccctga ccatcagcag cctgcagccc 240
gaggacttcg ccacctacta ctgccagcag ggcaacacac tgccctggac cttcggccag 300
ggcacaaagg tggagatcaa gcgtacggtg gctgcaccat ctgtcttcat cttcccgcca 360
tctgatgagc agttgaaatc tggaactgcc tctgttgtgt gcctgctgaa taacttctat 420
cccagagagg ccaaagtaca gtggaaggtg gataacgccc tccaatcggg taactcccag 480
gagagtgtca cagagcagga cagcaaggac agcacctaca gcctcagcag caccctgacg 540
ctgagcaaag cagactacga gaaacacaaa gtctacgcct gcgaagtcac ccatcagggc 600
ctgagctcgc ccgtcacaaa gagcttcaac aggggagagt gttag 645

SEQ ID NO: 13
<212> PRT
<223> V9 (scFab)-LC007 (VH-CH1)-Fc(knob) wt
Asp Ile Gln Met Thr Gln Ser Pro Ser Ser Leu Ser Ala Ser Val Gly
1 5 10 15
Asp Arg Val Thr Ile Thr Cys Arg Ala Ser Gln Asp Ile Arg Asn Tyr
20 25 30
Leu Asn Trp Tyr Gln Gln Lys Pro Gly Lys Ala Pro Lys Leu Leu Ile
35 40 45
Tyr Tyr Thr Ser Arg Leu Glu Ser Gly Val Pro Ser Arg Phe Ser Gly
50 55 60
Ser Gly Ser Gly Thr Asp Tyr Thr Leu Thr Ile Ser Ser Leu Gln Pro
65 70 75 80
Glu Asp Phe Ala Thr Tyr Tyr Cys Gln Gln Gly Asn Thr Leu Pro Trp
85 90 95
Thr Phe Gly Gln Gly Thr Lys Val Glu Ile Lys Arg Thr Val Ala Ala
100 105 110
Pro Ser Val Phe Ile Phe Pro Pro Ser Asp Glu Gln Leu Lys Ser Gly
115 120 125
Thr Ala Ser Val Val Cys Leu Leu Asn Asn Phe Tyr Pro Arg Glu Ala
130 135 140
Lys Val Gln Trp Lys Val Asp Asn Ala Leu Gln Ser Gly Asn Ser Gln
145 150 155 160
Glu Ser Val Thr Glu Gln Asp Ser Lys Asp Ser Thr Tyr Ser Leu Ser
165 170 175
Ser Thr Leu Thr Leu Ser Lys Ala Asp Tyr Glu Lys His Lys Val Tyr
180 185 190
Ala Cys Glu Val Thr His Gln Gly Leu Ser Ser Pro Val Thr Lys Ser
195 200 205
Phe Asn Arg Gly Glu Cys Ser Gly Gly Gly Ser Gly Gly Gly Ser Glu
210 215 220
Gly Gly Gly Ser Glu Gly Gly Gly Ser Glu Gly Gly Gly Ser Glu Gly
225 230 235 240
Gly Gly Ser Gly Gly Gly Ser Gly Glu Val Gln Leu Val Glu Ser Gly
245 250 255
Gly Gly Leu Val Gln Pro Gly Gly Ser Leu Arg Leu Ser Cys Ala Ala
260 265 270
Ser Gly Tyr Ser Phe Thr Gly Tyr Thr Met Asn Trp Val Arg Gln Ala
275 280 285
Pro Gly Lys Gly Leu Glu Trp Val Ala Leu Ile Asn Pro Tyr Lys Gly
290 295 300
Val Ser Thr Tyr Asn Gln Lys Phe Lys Asp Arg Phe Thr Ile Ser Val
305 310 315 320
Asp Lys Ser Lys Asn Thr Ala Tyr Leu Gln Met Asn Ser Leu Arg Ala
325 330 335
Glu Asp Thr Ala Val Tyr Tyr Cys Ala Arg Ser Gly Tyr Tyr Gly Asp
340 345 350
Ser Asp Trp Tyr Phe Asp Val Trp Gly Gln Gly Thr Leu Val Thr Val
355 360 365
Ser Ser Ala Ser Thr Lys Gly Pro Ser Val Phe Pro Leu Ala Pro Ser
370 375 380
Ser Lys Ser Thr Ser Gly Gly Thr Ala Ala Leu Gly Cys Leu Val Lys
385 390 395 400
Asp Tyr Phe Pro Glu Pro Val Thr Val Ser Trp Asn Ser Gly Ala Leu
405 410 415
Thr Ser Gly Val His Thr Phe Pro Ala Val Leu Gln Ser Ser Gly Leu
420 425 430
Tyr Ser Leu Ser Ser Val Val Thr Val Pro Ser Ser Ser Leu Gly Thr
435 440 445
Gln Thr Tyr Ile Cys Asn Val Asn His Lys Pro Ser Asn Thr Lys Val
450 455 460
Asp Lys Lys Val Glu Pro Lys Ser Cys Asp Gly Gly Gly Gly Ser Gly
465 470 475 480
Gly Gly Gly Ser Glu Val Gln Leu Gln Glu Ser Gly Pro Gly Leu Val
485 490 495
Lys Pro Ser Gln Ser Leu Ser Leu Thr Cys Ser Val Thr Gly Tyr Ser
500 505 510
Ile Thr Ser Gly Tyr Tyr Trp Asn Trp Ile Arg Gln Phe Pro Gly Asn
515 520 525
Lys Leu Glu Trp Met Gly Tyr Ile Thr Tyr Asp Gly Ser Asn Asn Tyr
530 535 540
Asn Pro Ser Leu Lys Asn Arg Ile Ser Ile Thr Arg Asp Thr Ser Lys
545 550 555 560
Asn Gln Phe Phe Leu Lys Leu Asn Ser Val Thr Thr Glu Asp Thr Ala
565 570 575
Thr Tyr Tyr Cys Ala Asp Phe Asp Tyr Trp Gly Gln Gly Thr Thr Leu
580 585 590
Thr Val Ser Ser Ala Ser Thr Lys Gly Pro Ser Val Phe Pro Leu Ala
595 600 605
Pro Ser Ser Lys Ser Thr Ser Gly Gly Thr Ala Ala Leu Gly Cys Leu
610 615 620
Val Lys Asp Tyr Phe Pro Glu Pro Val Thr Val Ser Trp Asn Ser Gly
625 630 635 640
Ala Leu Thr Ser Gly Val His Thr Phe Pro Ala Val Leu Gln Ser Ser
645 650 655
Gly Leu Tyr Ser Leu Ser Ser Val Val Thr Val Pro Ser Ser Ser Leu
660 665 670
Gly Thr Gln Thr Tyr Ile Cys Asn Val Asn His Lys Pro Ser Asn Thr
675 680 685
Lys Val Asp Lys Lys Val Glu Pro Lys Ser Cys Asp Lys Thr His Thr
690 695 700
Cys Pro Pro Cys Pro Ala Pro Glu Leu Leu Gly Gly Pro Ser Val Phe
705 710 715 720
Leu Phe Pro Pro Lys Pro Lys Asp Thr Leu Met Ile Ser Arg Thr Pro
725 730 735
Glu Val Thr Cys Val Val Val Asp Val Ser His Glu Asp Pro Glu Val
740 745 750
Lys Phe Asn Trp Tyr Val Asp Gly Val Glu Val His Asn Ala Lys Thr
755 760 765
Lys Pro Arg Glu Glu Gln Tyr Asn Ser Thr Tyr Arg Val Val Ser Val
770 775 780
Leu Thr Val Leu His Gln Asp Trp Leu Asn Gly Lys Glu Tyr Lys Cys
785 790 795 800
Lys Val Ser Asn Lys Ala Leu Pro Ala Pro Ile Glu Lys Thr Ile Ser
805 810 815
Lys Ala Lys Gly Gln Pro Arg Glu Pro Gln Val Tyr Thr Leu Pro Pro
820 825 830
Cys Arg Asp Glu Leu Thr Lys Asn Gln Val Ser Leu Trp Cys Leu Val
835 840 845
Lys Gly Phe Tyr Pro Ser Asp Ile Ala Val Glu Trp Glu Ser Asn Gly
850 855 860
Gln Pro Glu Asn Asn Tyr Lys Thr Thr Pro Pro Val Leu Asp Ser Asp
865 870 875 880
Gly Ser Phe Phe Leu Tyr Ser Lys Leu Thr Val Asp Lys Ser Arg Trp
885 890 895
Gln Gln Gly Asn Val Phe Ser Cys Ser Val Met His Glu Ala Leu His
900 905 910
Asn His Tyr Thr Gln Lys Ser Leu Ser Leu Ser Pro Gly Lys
915 920 925

SEQ ID NO: 14
<212> DNA
<223> V9 (scFab)-LC007 (VH-CH1)-Fc(knob) wt
gacatccaga tgacccagag cccctctagc ctgagcgcca gcgtgggcga cagagtgacc 60
atcacctgtc gggccagcca ggacatcaga aactacctga actggtatca gcagaagccc 120
ggcaaggccc ccaagctgct gatctactac acctctagac tggaaagcgg cgtgcccagc 180
cggtttagcg gcagcggctc cggcaccgac tacaccctga ccatcagcag cctgcagccc 240
gaggacttcg ccacctacta ctgccagcag ggcaacacac tcccctggac cttcggccag 300
ggcaccaagg tggagatcaa gcgtacggtg gccgctccca gcgtgttcat cttccccccc 360
agcgacgagc agctgaagtc cggcaccgcc agcgtcgtgt gcctgctgaa caacttctac 420
ccccgggagg ccaaggtgca gtggaaggtg gacaacgccc tgcagagcgg caacagccag 480
gaaagcgtca ccgagcagga cagcaaggac tccacctaca gcctgagcag caccctgacc 540
ctgagcaagg ccgactacga gaagcacaag gtgtacgcct gcgaagtgac ccaccagggc 600
ctgtccagcc ccgtgaccaa gagcttcaac cggggcgagt gcagcggcgg aggctctgga 660
ggcggctctg aaggcggagg aagtgagggc ggaggctcag aaggcggcgg aagcgaaggt 720
ggcggctctg gcggcggatc cggcgaggtg cagctggtcg agtccggcgg aggcctggtg 780
cagcctggcg gcagcctgag actgagctgc gccgccagcg gctacagctt caccggctac 840
accatgaact gggtccggca ggctcctggc aagggcctcg aatgggtggc cctgatcaac 900
ccctacaagg gcgtgagcac ctacaaccag aagttcaagg accggttcac catcagcgtg 960
gacaagagca agaacaccgc ctatctgcag atgaacagcc tgcgggccga ggacaccgcc 1020
gtgtactact gcgccagaag cggctactac ggcgacagcg actggtactt cgacgtgtgg 1080
ggccagggca cactggtcac cgtgtccagc gctagcacca agggcccctc cgtgttcccc 1140
ctggccccca gcagcaagag caccagcggc ggcacagccg ccctcggctg cctggtcaag 1200
gactacttcc ccgagcccgt gaccgtgtcc tggaacagcg gagccctgac ctccggcgtg 1260
cacaccttcc ccgccgtgct gcagagcagc ggcctgtaca gcctgtccag cgtggtcacc 1320
gtgccctcca gcagcctggg cacccagacc tacatctgca acgtgaacca caagcccagc 1380
aataccaagg tggacaagaa ggtggagccc aagagctgcg acggcggtgg tggctccgga 1440
ggcggtggat ctgaagtgca gctgcaggaa agcggccctg gcctggtcaa gcccagccag 1500
agcctgagcc tgacctgtag cgtgaccggc tactccatca cctccggcta ctactggaat 1560
tggattcggc agttccccgg caacaagctg gaatggatgg gctacatcac ctacgacggc 1620
agcaacaact acaaccccag cctgaagaac cggatcagca tcacccggga caccagcaag 1680
aaccagttct tcctgaagtt gaattctgtg actactgagg acacagctac atattactgt 1740
gcggactttg actactgggg ccaaggcacc actctcacag tctcctcagc tagcaccaag 1800
ggcccatcgg tcttccccct ggcaccctcc tccaagagca cctctggggg cacagcggcc 1860
ctgggctgcc tggtcaagga ctacttcccc gaaccggtga cggtgtcgtg gaactcaggc 1920
gccctgacca gcggcgtgca caccttcccg gctgtcctac agtcctcagg actctactcc 1980
ctcagcagcg tggtgaccgt gccctccagc agcttgggca cccagaccta catctgcaac 2040
gtgaatcaca agcccagcaa caccaaggtg gacaagaaag ttgagcccaa atcttgtgac 2100
aaaactcaca catgcccacc gtgcccagca cctgaactcc tggggggacc gtcagtcttc 2160
ctcttccccc caaaacccaa ggacaccctc atgatctccc ggacccctga ggtcacatgc 2220
gtggtggtgg acgtgagcca cgaagaccct gaggtcaagt tcaactggta cgtggacggc 2280
gtggaggtgc ataatgccaa gacaaagccg cgggaggagc agtacaacag cacgtaccgt 2340
gtggtcagcg tcctcaccgt cctgcaccag gactggctga atggcaagga gtacaagtgc 2400
aaggtctcca acaaagccct cccagccccc atcgagaaaa ccatctccaa agccaaaggg 2460
cagccccgag aaccacaggt gtacaccctg cccccatgcc gggatgagct gaccaagaac 2520
caggtcagcc tgtggtgcct ggtcaaaggc ttctatccca gcgacatcgc cgtggagtgg 2580
gagagcaatg ggcagccgga gaacaactac aagaccacgc ctcccgtgct ggactccgac 2640
ggctccttct tcctctacag caagctcacc gtggacaaga gcaggtggca gcaggggaac 2700
gtcttctcat gctccgtgat gcatgaggct ctgcacaacc actacacgca gaagagcctc 2760
tccctgtctc cgggtaaatg a 2781

SEQ ID NO: 15
<212> PRT
<223> LC007 (VH-CH1)-Fc(hole) wt
Glu Val Gln Leu Gln Glu Ser Gly Pro Gly Leu Val Lys Pro Ser Gln
1 5 10 15
Ser Leu Ser Leu Thr Cys Ser Val Thr Gly Tyr Ser Ile Thr Ser Gly
20 25 30
Tyr Tyr Trp Asn Trp Ile Arg Gln Phe Pro Gly Asn Lys Leu Glu Trp
35 40 45
Met Gly Tyr Ile Thr Tyr Asp Gly Ser Asn Asn Tyr Asn Pro Ser Leu
50 55 60
Lys Asn Arg Ile Ser Ile Thr Arg Asp Thr Ser Lys Asn Gln Phe Phe
65 70 75 80
Leu Lys Leu Asn Ser Val Thr Thr Glu Asp Thr Ala Thr Tyr Tyr Cys
85 90 95
Ala Asp Phe Asp Tyr Trp Gly Gln Gly Thr Thr Leu Thr Val Ser Ser
100 105 110
Ala Ser Thr Lys Gly Pro Ser Val Phe Pro Leu Ala Pro Ser Ser Lys
115 120 125
Ser Thr Ser Gly Gly Thr Ala Ala Leu Gly Cys Leu Val Lys Asp Tyr
130 135 140
Phe Pro Glu Pro Val Thr Val Ser Trp Asn Ser Gly Ala Leu Thr Ser
145 150 155 160
Gly Val His Thr Phe Pro Ala Val Leu Gln Ser Ser Gly Leu Tyr Ser
165 170 175
Leu Ser Ser Val Val Thr Val Pro Ser Ser Ser Leu Gly Thr Gln Thr
180 185 190
Tyr Ile Cys Asn Val Asn His Lys Pro Ser Asn Thr Lys Val Asp Lys
195 200 205
Lys Val Glu Pro Lys Ser Cys Asp Lys Thr His Thr Cys Pro Pro Cys
210 215 220
Pro Ala Pro Glu Leu Leu Gly Gly Pro Ser Val Phe Leu Phe Pro Pro
225 230 235 240
Lys Pro Lys Asp Thr Leu Met Ile Ser Arg Thr Pro Glu Val Thr Cys
245 250 255
Val Val Val Asp Val Ser His Glu Asp Pro Glu Val Lys Phe Asn Trp
260 265 270
Tyr Val Asp Gly Val Glu Val His Asn Ala Lys Thr Lys Pro Arg Glu
275 280 285
Glu Gln Tyr Asn Ser Thr Tyr Arg Val Val Ser Val Leu Thr Val Leu
290 295 300
His Gln Asp Trp Leu Asn Gly Lys Glu Tyr Lys Cys Lys Val Ser Asn
305 310 315 320
Lys Ala Leu Pro Ala Pro Ile Glu Lys Thr Ile Ser Lys Ala Lys Gly
325 330 335
Gln Pro Arg Glu Pro Gln Val Cys Thr Leu Pro Pro Ser Arg Asp Glu
340 345 350
Leu Thr Lys Asn Gln Val Ser Leu Ser Cys Ala Val Lys Gly Phe Tyr
355 360 365
Pro Ser Asp Ile Ala Val Glu Trp Glu Ser Asn Gly Gln Pro Glu Asn
370 375 380
Asn Tyr Lys Thr Thr Pro Pro Val Leu Asp Ser Asp Gly Ser Phe Phe
385 390 395 400
Leu Val Ser Lys Leu Thr Val Asp Lys Ser Arg Trp Gln Gln Gly Asn
405 410 415
Val Phe Ser Cys Ser Val Met His Glu Ala Leu His Asn His Tyr Thr
420 425 430
Gln Lys Ser Leu Ser Leu Ser Pro Gly Lys
435 440

SEQ ID NO: 16
<212> DNA
<223> LC007 (VH-CH1)-Fc(hole) wt
gaggtccagc tgcaggagtc aggacctggc ctcgtgaaac cttctcagtc tctgtctctc 60
acctgctctg tcactggcta ctccatcacc agtggttatt actggaactg gatccggcag 120
tttccaggaa acaagctgga atggatgggc tacataacct acgacggtag caataactac 180
aacccatctc tcaaaaatcg aatctccatc actcgtgaca catctaagaa ccagtttttc 240
ctgaagttga attctgtgac tactgaggac acagctacat attactgtgc ggactttgac 300
tactggggcc aaggcaccac tctcacagtc tcctcagcta gcaccaaggg cccaagcgtg 360
ttccctctgg cccccagcag caagagcaca agcggcggaa cagccgccct gggctgcctg 420
gtcaaggact acttccccga gcccgtgaca gtgtcctgga acagcggagc cctgaccagc 480
ggcgtgcaca cctttccagc cgtgctgcag agcagcggcc tgtacagcct gagcagcgtg 540
gtcacagtgc ctagcagcag cctgggcacc cagacctaca tctgcaacgt gaaccacaag 600
cccagcaaca ccaaggtgga caagaaggtg gagcccaaga gctgcgacaa gacccacacc 660
tgtccccctt gtcctgcccc tgagctgctg ggcggaccca gcgtgttcct gttcccccca 720
aagcccaagg acaccctgat gatcagccgg acccccgaag tgacctgcgt ggtggtggac 780
gtgtcccacg aggaccctga agtgaagttc aattggtacg tggacggcgt ggaggtgcac 840
aatgccaaga ccaagccccg ggaggaacag tacaacagca cctaccgggt ggtgtccgtg 900
ctgaccgtgc tgcaccagga ctggctgaac ggcaaagagt acaagtgcaa ggtctccaac 960
aaggccctgc ctgcccccat cgagaaaacc atcagcaagg ccaagggcca gcccagagaa 1020
ccccaggtgt gcaccctgcc ccccagcaga gatgagctga ccaagaacca ggtgtccctg 1080
agctgtgccg tcaagggctt ctaccccagc gatatcgccg tggagtggga gagcaacggc 1140
cagcctgaga acaactacaa gaccaccccc cctgtgctgg acagcgacgg cagcttcttc 1200
ctggtgtcca aactgaccgt ggacaagagc cggtggcagc agggcaacgt gttcagctgc 1260
agcgtgatgc acgaggccct gcacaaccac tacacccaga agtccctgag cctgagcccc 1320
ggcaagtga 1329

SEQ ID NO: 17
<212> PRT
<223> V9 (scFab)-LC007 (VH-CH1)-Fc(knob) LALA
Asp Ile Gln Met Thr Gln Ser Pro Ser Ser Leu Ser Ala Ser Val Gly
1 5 10 15
Asp Arg Val Thr Ile Thr Cys Arg Ala Ser Gln Asp Ile Arg Asn Tyr
20 25 30
Leu Asn Trp Tyr Gln Gln Lys Pro Gly Lys Ala Pro Lys Leu Leu Ile
35 40 45
Tyr Tyr Thr Ser Arg Leu Glu Ser Gly Val Pro Ser Arg Phe Ser Gly
50 55 60
Ser Gly Ser Gly Thr Asp Tyr Thr Leu Thr Ile Ser Ser Leu Gln Pro
65 70 75 80
Glu Asp Phe Ala Thr Tyr Tyr Cys Gln Gln Gly Asn Thr Leu Pro Trp
85 90 95
Thr Phe Gly Gln Gly Thr Lys Val Glu Ile Lys Arg Thr Val Ala Ala
100 105 110
Pro Ser Val Phe Ile Phe Pro Pro Ser Asp Glu Gln Leu Lys Ser Gly
115 120 125
Thr Ala Ser Val Val Cys Leu Leu Asn Asn Phe Tyr Pro Arg Glu Ala
130 135 140
Lys Val Gln Trp Lys Val Asp Asn Ala Leu Gln Ser Gly Asn Ser Gln
145 150 155 160
Glu Ser Val Thr Glu Gln Asp Ser Lys Asp Ser Thr Tyr Ser Leu Ser
165 170 175
Ser Thr Leu Thr Leu Ser Lys Ala Asp Tyr Glu Lys His Lys Val Tyr
180 185 190
Ala Cys Glu Val Thr His Gln Gly Leu Ser Ser Pro Val Thr Lys Ser
195 200 205
Phe Asn Arg Gly Glu Cys Ser Gly Gly Gly Ser Gly Gly Gly Ser Glu
210 215 220
Gly Gly Gly Ser Glu Gly Gly Gly Ser Glu Gly Gly Gly Ser Glu Gly
225 230 235 240
Gly Gly Ser Gly Gly Gly Ser Gly Glu Val Gln Leu Val Glu Ser Gly
245 250 255
Gly Gly Leu Val Gln Pro Gly Gly Ser Leu Arg Leu Ser Cys Ala Ala
260 265 270
Ser Gly Tyr Ser Phe Thr Gly Tyr Thr Met Asn Trp Val Arg Gln Ala
275 280 285
Pro Gly Lys Gly Leu Glu Trp Val Ala Leu Ile Asn Pro Tyr Lys Gly
290 295 300
Val Ser Thr Tyr Asn Gln Lys Phe Lys Asp Arg Phe Thr Ile Ser Val
305 310 315 320
Asp Lys Ser Lys Asn Thr Ala Tyr Leu Gln Met Asn Ser Leu Arg Ala
325 330 335
Glu Asp Thr Ala Val Tyr Tyr Cys Ala Arg Ser Gly Tyr Tyr Gly Asp
340 345 350
Ser Asp Trp Tyr Phe Asp Val Trp Gly Gln Gly Thr Leu Val Thr Val
355 360 365
Ser Ser Ala Ser Thr Lys Gly Pro Ser Val Phe Pro Leu Ala Pro Ser
370 375 380
Ser Lys Ser Thr Ser Gly Gly Thr Ala Ala Leu Gly Cys Leu Val Lys
385 390 395 400
Asp Tyr Phe Pro Glu Pro Val Thr Val Ser Trp Asn Ser Gly Ala Leu
405 410 415
Thr Ser Gly Val His Thr Phe Pro Ala Val Leu Gln Ser Ser Gly Leu
420 425 430
Tyr Ser Leu Ser Ser Val Val Thr Val Pro Ser Ser Ser Leu Gly Thr
435 440 445
Gln Thr Tyr Ile Cys Asn Val Asn His Lys Pro Ser Asn Thr Lys Val
450 455 460
Asp Lys Lys Val Glu Pro Lys Ser Cys Asp Gly Gly Gly Gly Ser Gly
465 470 475 480
Gly Gly Gly Ser Glu Val Gln Leu Gln Glu Ser Gly Pro Gly Leu Val
485 490 495
Lys Pro Ser Gln Ser Leu Ser Leu Thr Cys Ser Val Thr Gly Tyr Ser
500 505 510
Ile Thr Ser Gly Tyr Tyr Trp Asn Trp Ile Arg Gln Phe Pro Gly Asn
515 520 525
Lys Leu Glu Trp Met Gly Tyr Ile Thr Tyr Asp Gly Ser Asn Asn Tyr
530 535 540
Asn Pro Ser Leu Lys Asn Arg Ile Ser Ile Thr Arg Asp Thr Ser Lys
545 550 555 560
Asn Gln Phe Phe Leu Lys Leu Asn Ser Val Thr Thr Glu Asp Thr Ala
565 570 575
Thr Tyr Tyr Cys Ala Asp Phe Asp Tyr Trp Gly Gln Gly Thr Thr Leu
580 585 590
Thr Val Ser Ser Ala Ser Thr Lys Gly Pro Ser Val Phe Pro Leu Ala
595 600 605
Pro Ser Ser Lys Ser Thr Ser Gly Gly Thr Ala Ala Leu Gly Cys Leu
610 615 620
Val Lys Asp Tyr Phe Pro Glu Pro Val Thr Val Ser Trp Asn Ser Gly
625 630 635 640
Ala Leu Thr Ser Gly Val His Thr Phe Pro Ala Val Leu Gln Ser Ser
645 650 655
Gly Leu Tyr Ser Leu Ser Ser Val Val Thr Val Pro Ser Ser Ser Leu
660 665 670
Gly Thr Gln Thr Tyr Ile Cys Asn Val Asn His Lys Pro Ser Asn Thr
675 680 685
Lys Val Asp Lys Lys Val Glu Pro Lys Ser Cys Asp Lys Thr His Thr
690 695 700
Cys Pro Pro Cys Pro Ala Pro Glu Ala Ala Gly Gly Pro Ser Val Phe
705 710 715 720
Leu Phe Pro Pro Lys Pro Lys Asp Thr Leu Met Ile Ser Arg Thr Pro
725 730 735
Glu Val Thr Cys Val Val Val Asp Val Ser His Glu Asp Pro Glu Val
740 745 750
Lys Phe Asn Trp Tyr Val Asp Gly Val Glu Val His Asn Ala Lys Thr
755 760 765
Lys Pro Arg Glu Glu Gln Tyr Asn Ser Thr Tyr Arg Val Val Ser Val
770 775 780
Leu Thr Val Leu His Gln Asp Trp Leu Asn Gly Lys Glu Tyr Lys Cys
785 790 795 800
Lys Val Ser Asn Lys Ala Leu Pro Ala Pro Ile Glu Lys Thr Ile Ser
805 810 815
Lys Ala Lys Gly Gln Pro Arg Glu Pro Gln Val Tyr Thr Leu Pro Pro
820 825 830
Cys Arg Asp Glu Leu Thr Lys Asn Gln Val Ser Leu Trp Cys Leu Val
835 840 845
Lys Gly Phe Tyr Pro Ser Asp Ile Ala Val Glu Trp Glu Ser Asn Gly
850 855 860
Gln Pro Glu Asn Asn Tyr Lys Thr Thr Pro Pro Val Leu Asp Ser Asp
865 870 875 880
Gly Ser Phe Phe Leu Tyr Ser Lys Leu Thr Val Asp Lys Ser Arg Trp
885 890 895
Gln Gln Gly Asn Val Phe Ser Cys Ser Val Met His Glu Ala Leu His
900 905 910
Asn His Tyr Thr Gln Lys Ser Leu Ser Leu Ser Pro Gly Lys
915 920 925

SEQ ID NO: 18
<212> DNA
<223> V9 (scFab)-LC007 (VH-CH1)-Fc(knob) LALA
gacatccaga tgacccagag cccctctagc ctgagcgcca gcgtgggcga cagagtgacc 60
atcacctgtc gggccagcca ggacatcaga aactacctga actggtatca gcagaagccc 120
ggcaaggccc ccaagctgct gatctactac acctctagac tggaaagcgg cgtgcccagc 180
cggtttagcg gcagcggctc cggcaccgac tacaccctga ccatcagcag cctgcagccc 240
gaggacttcg ccacctacta ctgccagcag ggcaacacac tcccctggac cttcggccag 300
ggcaccaagg tggagatcaa gcgtacggtg gccgctccca gcgtgttcat cttccccccc 360
agcgacgagc agctgaagtc cggcaccgcc agcgtcgtgt gcctgctgaa caacttctac 420
ccccgggagg ccaaggtgca gtggaaggtg gacaacgccc tgcagagcgg caacagccag 480
gaaagcgtca ccgagcagga cagcaaggac tccacctaca gcctgagcag caccctgacc 540
ctgagcaagg ccgactacga gaagcacaag gtgtacgcct gcgaagtgac ccaccagggc 600
ctgtccagcc ccgtgaccaa gagcttcaac cggggcgagt gcagcggcgg aggctctgga 660
ggcggctctg aaggcggagg aagtgagggc ggaggctcag aaggcggcgg aagcgaaggt 720
ggcggctctg gcggcggatc cggcgaggtg cagctggtcg agtccggcgg aggcctggtg 780
cagcctggcg gcagcctgag actgagctgc gccgccagcg gctacagctt caccggctac 840
accatgaact gggtccggca ggctcctggc aagggcctcg aatgggtggc cctgatcaac 900
ccctacaagg gcgtgagcac ctacaaccag aagttcaagg accggttcac catcagcgtg 960
gacaagagca agaacaccgc ctatctgcag atgaacagcc tgcgggccga ggacaccgcc 1020
gtgtactact gcgccagaag cggctactac ggcgacagcg actggtactt cgacgtgtgg 1080
ggccagggca cactggtcac cgtgtccagc gctagcacca agggcccctc cgtgttcccc 1140
ctggccccca gcagcaagag caccagcggc ggcacagccg ccctcggctg cctggtcaag 1200
gactacttcc ccgagcccgt gaccgtgtcc tggaacagcg gagccctgac ctccggcgtg 1260
cacaccttcc ccgccgtgct gcagagcagc ggcctgtaca gcctgtccag cgtggtcacc 1320
gtgccctcca gcagcctggg cacccagacc tacatctgca acgtgaacca caagcccagc 1380
aataccaagg tggacaagaa ggtggagccc aagagctgcg acggcggtgg tggctccgga 1440
ggcggtggat ctgaagtgca gctgcaggaa agcggccctg gcctggtcaa gcccagccag 1500
agcctgagcc tgacctgtag cgtgaccggc tactccatca cctccggcta ctactggaat 1560
tggattcggc agttccccgg caacaagctg gaatggatgg gctacatcac ctacgacggc 1620
agcaacaact acaaccccag cctgaagaac cggatcagca tcacccggga caccagcaag 1680
aaccagttct tcctgaagtt gaattctgtg actactgagg acacagctac atattactgt 1740
gcggactttg actactgggg ccaaggcacc actctcacag tctcctcagc tagcaccaag 1800
ggcccatcgg tcttccccct ggcaccctcc tccaagagca cctctggggg cacagcggcc 1860
ctgggctgcc tggtcaagga ctacttcccc gaaccggtga cggtgtcgtg gaactcaggc 1920
gccctgacca gcggcgtgca caccttcccg gctgtcctac agtcctcagg actctactcc 1980
ctcagcagcg tggtgaccgt gccctccagc agcttgggca cccagaccta catctgcaac 2040
gtgaatcaca agcccagcaa caccaaggtg gacaagaaag ttgagcccaa atcttgtgac 2100
aaaactcaca catgcccacc gtgcccagca cctgaagctg cagggggacc gtcagtcttc 2160
ctcttccccc caaaacccaa ggacaccctc atgatctccc ggacccctga ggtcacatgc 2220
gtggtggtgg acgtgagcca cgaagaccct gaggtcaagt tcaactggta cgtggacggc 2280
gtggaggtgc ataatgccaa gacaaagccg cgggaggagc agtacaacag cacgtaccgt 2340
gtggtcagcg tcctcaccgt cctgcaccag gactggctga atggcaagga gtacaagtgc 2400
aaggtctcca acaaagccct cccagccccc atcgagaaaa ccatctccaa agccaaaggg 2460
cagccccgag aaccacaggt gtacaccctg cccccatgcc gggatgagct gaccaagaac 2520
caggtcagcc tgtggtgcct ggtcaaaggc ttctatccca gcgacatcgc cgtggagtgg 2580
gagagcaatg ggcagccgga gaacaactac aagaccacgc ctcccgtgct ggactccgac 2640
ggctccttct tcctctacag caagctcacc gtggacaaga gcaggtggca gcaggggaac 2700
gtcttctcat gctccgtgat gcatgaggct ctgcacaacc actacacgca gaagagcctc 2760
tccctgtctc cgggtaaatg a 2781

SEQ ID NO: 19
<212> PRT
<223> LC007 (VH-CH1)-Fc(hole) LALA
Glu Val Gln Leu Gln Glu Ser Gly Pro Gly Leu Val Lys Pro Ser Gln
1 5 10 15
Ser Leu Ser Leu Thr Cys Ser Val Thr Gly Tyr Ser Ile Thr Ser Gly
20 25 30
Tyr Tyr Trp Asn Trp Ile Arg Gln Phe Pro Gly Asn Lys Leu Glu Trp
35 40 45
Met Gly Tyr Ile Thr Tyr Asp Gly Ser Asn Asn Tyr Asn Pro Ser Leu
50 55 60
Lys Asn Arg Ile Ser Ile Thr Arg Asp Thr Ser Lys Asn Gln Phe Phe
65 70 75 80
Leu Lys Leu Asn Ser Val Thr Thr Glu Asp Thr Ala Thr Tyr Tyr Cys
85 90 95
Ala Asp Phe Asp Tyr Trp Gly Gln Gly Thr Thr Leu Thr Val Ser Ser
100 105 110
Ala Ser Thr Lys Gly Pro Ser Val Phe Pro Leu Ala Pro Ser Ser Lys
115 120 125
Ser Thr Ser Gly Gly Thr Ala Ala Leu Gly Cys Leu Val Lys Asp Tyr
130 135 140
Phe Pro Glu Pro Val Thr Val Ser Trp Asn Ser Gly Ala Leu Thr Ser
145 150 155 160
Gly Val His Thr Phe Pro Ala Val Leu Gln Ser Ser Gly Leu Tyr Ser
165 170 175
Leu Ser Ser Val Val Thr Val Pro Ser Ser Ser Leu Gly Thr Gln Thr
180 185 190
Tyr Ile Cys Asn Val Asn His Lys Pro Ser Asn Thr Lys Val Asp Lys
195 200 205
Lys Val Glu Pro Lys Ser Cys Asp Lys Thr His Thr Cys Pro Pro Cys
210 215 220
Pro Ala Pro Glu Ala Ala Gly Gly Pro Ser Val Phe Leu Phe Pro Pro
225 230 235 240
Lys Pro Lys Asp Thr Leu Met Ile Ser Arg Thr Pro Glu Val Thr Cys
245 250 255
Val Val Val Asp Val Ser His Glu Asp Pro Glu Val Lys Phe Asn Trp
260 265 270
Tyr Val Asp Gly Val Glu Val His Asn Ala Lys Thr Lys Pro Arg Glu
275 280 285
Glu Gln Tyr Asn Ser Thr Tyr Arg Val Val Ser Val Leu Thr Val Leu
290 295 300
His Gln Asp Trp Leu Asn Gly Lys Glu Tyr Lys Cys Lys Val Ser Asn
305 310 315 320
Lys Ala Leu Pro Ala Pro Ile Glu Lys Thr Ile Ser Lys Ala Lys Gly
325 330 335
Gln Pro Arg Glu Pro Gln Val Cys Thr Leu Pro Pro Ser Arg Asp Glu
340 345 350
Leu Thr Lys Asn Gln Val Ser Leu Ser Cys Ala Val Lys Gly Phe Tyr
355 360 365
Pro Ser Asp Ile Ala Val Glu Trp Glu Ser Asn Gly Gln Pro Glu Asn
370 375 380
Asn Tyr Lys Thr Thr Pro Pro Val Leu Asp Ser Asp Gly Ser Phe Phe
385 390 395 400
Leu Val Ser Lys Leu Thr Val Asp Lys Ser Arg Trp Gln Gln Gly Asn
405 410 415
Val Phe Ser Cys Ser Val Met His Glu Ala Leu His Asn His Tyr Thr
420 425 430
Gln Lys Ser Leu Ser Leu Ser Pro Gly Lys
435 440

SEQ ID NO: 20
<212> DNA
<223> LC007 (VH-CH1)-Fc(hole) LALA
gaggtccagc tgcaggagtc aggacctggc ctcgtgaaac cttctcagtc tctgtctctc 60
acctgctctg tcactggcta ctccatcacc agtggttatt actggaactg gatccggcag 120
tttccaggaa acaagctgga atggatgggc tacataacct acgacggtag caataactac 180
aacccatctc tcaaaaatcg aatctccatc actcgtgaca catctaagaa ccagtttttc 240
ctgaagttga attctgtgac tactgaggac acagctacat attactgtgc ggactttgac 300
tactggggcc aaggcaccac tctcacagtc tcctcagcta gcaccaaggg cccatcggtc 360
ttccccctgg caccctcctc caagagcacc tctgggggca cagcggccct gggctgcctg 420
gtcaaggact acttccccga accggtgacg gtgtcgtgga actcaggcgc cctgaccagc 480
ggcgtgcaca ccttcccggc tgtcctacag tcctcaggac tctactccct cagcagcgtg 540
gtgaccgtgc cctccagcag cttgggcacc cagacctaca tctgcaacgt gaatcacaag 600
cccagcaaca ccaaggtgga caagaaagtt gagcccaaat cttgtgacaa aactcacaca 660
tgcccaccgt gcccagcacc tgaagctgca gggggaccgt cagtcttcct cttcccccca 720
aaacccaagg acaccctcat gatctcccgg acccctgagg tcacatgcgt ggtggtggac 780
gtgagccacg aagaccctga ggtcaagttc aactggtacg tggacggcgt ggaggtgcat 840
aatgccaaga caaagccgcg ggaggagcag tacaacagca cgtaccgtgt ggtcagcgtc 900
ctcaccgtcc tgcaccagga ctggctgaat ggcaaggagt acaagtgcaa ggtctccaac 960
aaagccctcc cagcccccat cgagaaaacc atctccaaag ccaaagggca gccccgagaa 1020
ccacaggtgt gcaccctgcc cccatcccgg gatgagctga ccaagaacca ggtcagcctc 1080
tcgtgcgcag tcaaaggctt ctatcccagc gacatcgccg tggagtggga gagcaatggg 1140
cagccggaga acaactacaa gaccacgcct cccgtgctgg actccgacgg ctccttcttc 1200
ctcgtgagca agctcaccgt ggacaagagc aggtggcagc aggggaacgt cttctcatgc 1260
tccgtgatgc atgaggctct gcacaaccac tacacgcaga agagcctctc cctgtctccg 1320
ggtaaatga 1329

SEQ ID NO: 21
<212> PRT
<223> V9 (scFab)-LC007 (VH-CH1)-Fc(knob) P329G LALA
Asp Ile Gln Met Thr Gln Ser Pro Ser Ser Leu Ser Ala Ser Val Gly
1 5 10 15
Asp Arg Val Thr Ile Thr Cys Arg Ala Ser Gln Asp Ile Arg Asn Tyr
20 25 30
Leu Asn Trp Tyr Gln Gln Lys Pro Gly Lys Ala Pro Lys Leu Leu Ile
35 40 45
Tyr Tyr Thr Ser Arg Leu Glu Ser Gly Val Pro Ser Arg Phe Ser Gly
50 55 60
Ser Gly Ser Gly Thr Asp Tyr Thr Leu Thr Ile Ser Ser Leu Gln Pro
65 70 75 80
Glu Asp Phe Ala Thr Tyr Tyr Cys Gln Gln Gly Asn Thr Leu Pro Trp
85 90 95
Thr Phe Gly Gln Gly Thr Lys Val Glu Ile Lys Arg Thr Val Ala Ala
100 105 110
Pro Ser Val Phe Ile Phe Pro Pro Ser Asp Glu Gln Leu Lys Ser Gly
115 120 125
Thr Ala Ser Val Val Cys Leu Leu Asn Asn Phe Tyr Pro Arg Glu Ala
130 135 140
Lys Val Gln Trp Lys Val Asp Asn Ala Leu Gln Ser Gly Asn Ser Gln
145 150 155 160
Glu Ser Val Thr Glu Gln Asp Ser Lys Asp Ser Thr Tyr Ser Leu Ser
165 170 175
Ser Thr Leu Thr Leu Ser Lys Ala Asp Tyr Glu Lys His Lys Val Tyr
180 185 190
Ala Cys Glu Val Thr His Gln Gly Leu Ser Ser Pro Val Thr Lys Ser
195 200 205
Phe Asn Arg Gly Glu Cys Ser Gly Gly Gly Ser Gly Gly Gly Ser Glu
210 215 220
Gly Gly Gly Ser Glu Gly Gly Gly Ser Glu Gly Gly Gly Ser Glu Gly
225 230 235 240
Gly Gly Ser Gly Gly Gly Ser Gly Glu Val Gln Leu Val Glu Ser Gly
245 250 255
Gly Gly Leu Val Gln Pro Gly Gly Ser Leu Arg Leu Ser Cys Ala Ala
260 265 270
Ser Gly Tyr Ser Phe Thr Gly Tyr Thr Met Asn Trp Val Arg Gln Ala
275 280 285
Pro Gly Lys Gly Leu Glu Trp Val Ala Leu Ile Asn Pro Tyr Lys Gly
290 295 300
Val Ser Thr Tyr Asn Gln Lys Phe Lys Asp Arg Phe Thr Ile Ser Val
305 310 315 320
Asp Lys Ser Lys Asn Thr Ala Tyr Leu Gln Met Asn Ser Leu Arg Ala
325 330 335
Glu Asp Thr Ala Val Tyr Tyr Cys Ala Arg Ser Gly Tyr Tyr Gly Asp
340 345 350
Ser Asp Trp Tyr Phe Asp Val Trp Gly Gln Gly Thr Leu Val Thr Val
355 360 365
Ser Ser Ala Ser Thr Lys Gly Pro Ser Val Phe Pro Leu Ala Pro Ser
370 375 380
Ser Lys Ser Thr Ser Gly Gly Thr Ala Ala Leu Gly Cys Leu Val Lys
385 390 395 400
Asp Tyr Phe Pro Glu Pro Val Thr Val Ser Trp Asn Ser Gly Ala Leu
405 410 415
Thr Ser Gly Val His Thr Phe Pro Ala Val Leu Gln Ser Ser Gly Leu
420 425 430
Tyr Ser Leu Ser Ser Val Val Thr Val Pro Ser Ser Ser Leu Gly Thr
435 440 445
Gln Thr Tyr Ile Cys Asn Val Asn His Lys Pro Ser Asn Thr Lys Val
450 455 460
Asp Lys Lys Val Glu Pro Lys Ser Cys Asp Gly Gly Gly Gly Ser Gly
465 470 475 480
Gly Gly Gly Ser Glu Val Gln Leu Gln Glu Ser Gly Pro Gly Leu Val
485 490 495
Lys Pro Ser Gln Ser Leu Ser Leu Thr Cys Ser Val Thr Gly Tyr Ser
500 505 510
Ile Thr Ser Gly Tyr Tyr Trp Asn Trp Ile Arg Gln Phe Pro Gly Asn
515 520 525
Lys Leu Glu Trp Met Gly Tyr Ile Thr Tyr Asp Gly Ser Asn Asn Tyr
530 535 540
Asn Pro Ser Leu Lys Asn Arg Ile Ser Ile Thr Arg Asp Thr Ser Lys
545 550 555 560
Asn Gln Phe Phe Leu Lys Leu Asn Ser Val Thr Thr Glu Asp Thr Ala
565 570 575
Thr Tyr Tyr Cys Ala Asp Phe Asp Tyr Trp Gly Gln Gly Thr Thr Leu
580 585 590
Thr Val Ser Ser Ala Ser Thr Lys Gly Pro Ser Val Phe Pro Leu Ala
595 600 605
Pro Ser Ser Lys Ser Thr Ser Gly Gly Thr Ala Ala Leu Gly Cys Leu
610 615 620
Val Lys Asp Tyr Phe Pro Glu Pro Val Thr Val Ser Trp Asn Ser Gly
625 630 635 640
Ala Leu Thr Ser Gly Val His Thr Phe Pro Ala Val Leu Gln Ser Ser
645 650 655
Gly Leu Tyr Ser Leu Ser Ser Val Val Thr Val Pro Ser Ser Ser Leu
660 665 670
Gly Thr Gln Thr Tyr Ile Cys Asn Val Asn His Lys Pro Ser Asn Thr
675 680 685
Lys Val Asp Lys Lys Val Glu Pro Lys Ser Cys Asp Lys Thr His Thr
690 695 700
Cys Pro Pro Cys Pro Ala Pro Glu Ala Ala Gly Gly Pro Ser Val Phe
705 710 715 720
Leu Phe Pro Pro Lys Pro Lys Asp Thr Leu Met Ile Ser Arg Thr Pro
725 730 735
Glu Val Thr Cys Val Val Val Asp Val Ser His Glu Asp Pro Glu Val
740 745 750
Lys Phe Asn Trp Tyr Val Asp Gly Val Glu Val His Asn Ala Lys Thr
755 760 765
Lys Pro Arg Glu Glu Gln Tyr Asn Ser Thr Tyr Arg Val Val Ser Val
770 775 780
Leu Thr Val Leu His Gln Asp Trp Leu Asn Gly Lys Glu Tyr Lys Cys
785 790 795 800
Lys Val Ser Asn Lys Ala Leu Gly Ala Pro Ile Glu Lys Thr Ile Ser
805 810 815
Lys Ala Lys Gly Gln Pro Arg Glu Pro Gln Val Tyr Thr Leu Pro Pro
820 825 830
Cys Arg Asp Glu Leu Thr Lys Asn Gln Val Ser Leu Trp Cys Leu Val
835 840 845
Lys Gly Phe Tyr Pro Ser Asp Ile Ala Val Glu Trp Glu Ser Asn Gly
850 855 860
Gln Pro Glu Asn Asn Tyr Lys Thr Thr Pro Pro Val Leu Asp Ser Asp
865 870 875 880
Gly Ser Phe Phe Leu Tyr Ser Lys Leu Thr Val Asp Lys Ser Arg Trp
885 890 895
Gln Gln Gly Asn Val Phe Ser Cys Ser Val Met His Glu Ala Leu His
900 905 910
Asn His Tyr Thr Gln Lys Ser Leu Ser Leu Ser Pro Gly Lys
915 920 925

SEQ ID NO: 22
<212> DNA
<223> V9 (scFab)-LC007 (VH-CH1)-Fc(knob) P329G LALA
gacatccaga tgacccagag cccctctagc ctgagcgcca gcgtgggcga cagagtgacc 60
atcacctgtc gggccagcca ggacatcaga aactacctga actggtatca gcagaagccc 120
ggcaaggccc ccaagctgct gatctactac acctctagac tggaaagcgg cgtgcccagc 180
cggtttagcg gcagcggctc cggcaccgac tacaccctga ccatcagcag cctgcagccc 240
gaggacttcg ccacctacta ctgccagcag ggcaacacac tcccctggac cttcggccag 300
ggcaccaagg tggagatcaa gcgtacggtg gccgctccca gcgtgttcat cttccccccc 360
agcgacgagc agctgaagtc cggcaccgcc agcgtcgtgt gcctgctgaa caacttctac 420
ccccgggagg ccaaggtgca gtggaaggtg gacaacgccc tgcagagcgg caacagccag 480
gaaagcgtca ccgagcagga cagcaaggac tccacctaca gcctgagcag caccctgacc 540
ctgagcaagg ccgactacga gaagcacaag gtgtacgcct gcgaagtgac ccaccagggc 600
ctgtccagcc ccgtgaccaa gagcttcaac cggggcgagt gcagcggcgg aggctctgga 660
ggcggctctg aaggcggagg aagtgagggc ggaggctcag aaggcggcgg aagcgaaggt 720
ggcggctctg gcggcggatc cggcgaggtg cagctggtcg agtccggcgg aggcctggtg 780
cagcctggcg gcagcctgag actgagctgc gccgccagcg gctacagctt caccggctac 840
accatgaact gggtccggca ggctcctggc aagggcctcg aatgggtggc cctgatcaac 900
ccctacaagg gcgtgagcac ctacaaccag aagttcaagg accggttcac catcagcgtg 960
gacaagagca agaacaccgc ctatctgcag atgaacagcc tgcgggccga ggacaccgcc 1020
gtgtactact gcgccagaag cggctactac ggcgacagcg actggtactt cgacgtgtgg 1080
ggccagggca cactggtcac cgtgtccagc gctagcacca agggcccctc cgtgttcccc 1140
ctggccccca gcagcaagag caccagcggc ggcacagccg ccctcggctg cctggtcaag 1200
gactacttcc ccgagcccgt gaccgtgtcc tggaacagcg gagccctgac ctccggcgtg 1260
cacaccttcc ccgccgtgct gcagagcagc ggcctgtaca gcctgtccag cgtggtcacc 1320
gtgccctcca gcagcctggg cacccagacc tacatctgca acgtgaacca caagcccagc 1380
aataccaagg tggacaagaa ggtggagccc aagagctgcg acggcggtgg tggctccgga 1440
ggcggtggat ctgaagtgca gctgcaggaa agcggccctg gcctggtcaa gcccagccag 1500
agcctgagcc tgacctgtag cgtgaccggc tactccatca cctccggcta ctactggaat 1560
tggattcggc agttccccgg caacaagctg gaatggatgg gctacatcac ctacgacggc 1620
agcaacaact acaaccccag cctgaagaac cggatcagca tcacccggga caccagcaag 1680
aaccagttct tcctgaagtt gaattctgtg actactgagg acacagctac atattactgt 1740
gcggactttg actactgggg ccaaggcacc actctcacag tctcctcagc tagcaccaag 1800
ggcccatcgg tcttccccct ggcaccctcc tccaagagca cctctggggg cacagcggcc 1860
ctgggctgcc tggtcaagga ctacttcccc gaaccggtga cggtgtcgtg gaactcaggc 1920
gccctgacca gcggcgtgca caccttcccg gctgtcctac agtcctcagg actctactcc 1980
ctcagcagcg tggtgaccgt gccctccagc agcttgggca cccagaccta catctgcaac 2040
gtgaatcaca agcccagcaa caccaaggtg gacaagaaag ttgagcccaa atcttgtgac 2100
aaaactcaca catgcccacc gtgcccagca cctgaagctg cagggggacc gtcagtcttc 2160
ctcttccccc caaaacccaa ggacaccctc atgatctccc ggacccctga ggtcacatgc 2220
gtggtggtgg acgtgagcca cgaagaccct gaggtcaagt tcaactggta cgtggacggc 2280
gtggaggtgc ataatgccaa gacaaagccg cgggaggagc agtacaacag cacgtaccgt 2340
gtggtcagcg tcctcaccgt cctgcaccag gactggctga atggcaagga gtacaagtgc 2400
aaggtctcca acaaagccct cggcgccccc atcgagaaaa ccatctccaa agccaaaggg 2460
cagccccgag aaccacaggt gtacaccctg cccccatgcc gggatgagct gaccaagaac 2520
caggtcagcc tgtggtgcct ggtcaaaggc ttctatccca gcgacatcgc cgtggagtgg 2580
gagagcaatg ggcagccgga gaacaactac aagaccacgc ctcccgtgct ggactccgac 2640
ggctccttct tcctctacag caagctcacc gtggacaaga gcaggtggca gcaggggaac 2700
gtcttctcat gctccgtgat gcatgaggct ctgcacaacc actacacgca gaagagcctc 2760
tccctgtctc cgggtaaatg a 2781

SEQ ID NO: 23
<212> PRT
<223> LC007 (VH-CH1)-Fc(hole) P329G LALA
Glu Val Gln Leu Gln Glu Ser Gly Pro Gly Leu Val Lys Pro Ser Gln
1 5 10 15
Ser Leu Ser Leu Thr Cys Ser Val Thr Gly Tyr Ser Ile Thr Ser Gly
20 25 30
Tyr Tyr Trp Asn Trp Ile Arg Gln Phe Pro Gly Asn Lys Leu Glu Trp
35 40 45
Met Gly Tyr Ile Thr Tyr Asp Gly Ser Asn Asn Tyr Asn Pro Ser Leu
50 55 60
Lys Asn Arg Ile Ser Ile Thr Arg Asp Thr Ser Lys Asn Gln Phe Phe
65 70 75 80
Leu Lys Leu Asn Ser Val Thr Thr Glu Asp Thr Ala Thr Tyr Tyr Cys
85 90 95
Ala Asp Phe Asp Tyr Trp Gly Gln Gly Thr Thr Leu Thr Val Ser Ser
100 105 110
Ala Ser Thr Lys Gly Pro Ser Val Phe Pro Leu Ala Pro Ser Ser Lys
115 120 125
Ser Thr Ser Gly Gly Thr Ala Ala Leu Gly Cys Leu Val Lys Asp Tyr
130 135 140
Phe Pro Glu Pro Val Thr Val Ser Trp Asn Ser Gly Ala Leu Thr Ser
145 150 155 160
Gly Val His Thr Phe Pro Ala Val Leu Gln Ser Ser Gly Leu Tyr Ser
165 170 175
Leu Ser Ser Val Val Thr Val Pro Ser Ser Ser Leu Gly Thr Gln Thr
180 185 190
Tyr Ile Cys Asn Val Asn His Lys Pro Ser Asn Thr Lys Val Asp Lys
195 200 205
Lys Val Glu Pro Lys Ser Cys Asp Lys Thr His Thr Cys Pro Pro Cys
210 215 220
Pro Ala Pro Glu Ala Ala Gly Gly Pro Ser Val Phe Leu Phe Pro Pro
225 230 235 240
Lys Pro Lys Asp Thr Leu Met Ile Ser Arg Thr Pro Glu Val Thr Cys
245 250 255
Val Val Val Asp Val Ser His Glu Asp Pro Glu Val Lys Phe Asn Trp
260 265 270
Tyr Val Asp Gly Val Glu Val His Asn Ala Lys Thr Lys Pro Arg Glu
275 280 285
Glu Gln Tyr Asn Ser Thr Tyr Arg Val Val Ser Val Leu Thr Val Leu
290 295 300
His Gln Asp Trp Leu Asn Gly Lys Glu Tyr Lys Cys Lys Val Ser Asn
305 310 315 320
Lys Ala Leu Gly Ala Pro Ile Glu Lys Thr Ile Ser Lys Ala Lys Gly
325 330 335
Gln Pro Arg Glu Pro Gln Val Cys Thr Leu Pro Pro Ser Arg Asp Glu
340 345 350
Leu Thr Lys Asn Gln Val Ser Leu Ser Cys Ala Val Lys Gly Phe Tyr
355 360 365
Pro Ser Asp Ile Ala Val Glu Trp Glu Ser Asn Gly Gln Pro Glu Asn
370 375 380
Asn Tyr Lys Thr Thr Pro Pro Val Leu Asp Ser Asp Gly Ser Phe Phe
385 390 395 400
Leu Val Ser Lys Leu Thr Val Asp Lys Ser Arg Trp Gln Gln Gly Asn
405 410 415
Val Phe Ser Cys Ser Val Met His Glu Ala Leu His Asn His Tyr Thr
420 425 430
Gln Lys Ser Leu Ser Leu Ser Pro Gly Lys
435 440

SEQ ID NO: 24
<212> DNA
<223> LC007 (VH-CH1)-Fc(hole) P329G LALA
gaggtccagc tgcaggagtc aggacctggc ctcgtgaaac cttctcagtc tctgtctctc 60
acctgctctg tcactggcta ctccatcacc agtggttatt actggaactg gatccggcag 120
tttccaggaa acaagctgga atggatgggc tacataacct acgacggtag caataactac 180
aacccatctc tcaaaaatcg aatctccatc actcgtgaca catctaagaa ccagtttttc 240
ctgaagttga attctgtgac tactgaggac acagctacat attactgtgc ggactttgac 300
tactggggcc aaggcaccac tctcacagtc tcctcagcta gcaccaaggg cccatcggtc 360
ttccccctgg caccctcctc caagagcacc tctgggggca cagcggccct gggctgcctg 420
gtcaaggact acttccccga accggtgacg gtgtcgtgga actcaggcgc cctgaccagc 480
ggcgtgcaca ccttcccggc tgtcctacag tcctcaggac tctactccct cagcagcgtg 540
gtgaccgtgc cctccagcag cttgggcacc cagacctaca tctgcaacgt gaatcacaag 600
cccagcaaca ccaaggtgga caagaaagtt gagcccaaat cttgtgacaa aactcacaca 660
tgcccaccgt gcccagcacc tgaagctgca gggggaccgt cagtcttcct cttcccccca 720
aaacccaagg acaccctcat gatctcccgg acccctgagg tcacatgcgt ggtggtggac 780
gtgagccacg aagaccctga ggtcaagttc aactggtacg tggacggcgt ggaggtgcat 840
aatgccaaga caaagccgcg ggaggagcag tacaacagca cgtaccgtgt ggtcagcgtc 900
ctcaccgtcc tgcaccagga ctggctgaat ggcaaggagt acaagtgcaa ggtctccaac 960
aaagccctcg gcgcccccat cgagaaaacc atctccaaag ccaaagggca gccccgagaa 1020
ccacaggtgt gcaccctgcc cccatcccgg gatgagctga ccaagaacca ggtcagcctc 1080
tcgtgcgcag tcaaaggctt ctatcccagc gacatcgccg tggagtggga gagcaatggg 1140
cagccggaga acaactacaa gaccacgcct cccgtgctgg actccgacgg ctccttcttc 1200
ctcgtgagca agctcaccgt ggacaagagc aggtggcagc aggggaacgt cttctcatgc 1260
tccgtgatgc atgaggctct gcacaaccac tacacgcaga agagcctctc cctgtctccg 1320
ggtaaatga 1329

SEQ ID NO: 25
<212> PRT
<223> V9 (scFab)-LC007 (VH-CH1)-Fc(knob) P329G LALA N297D
Asp Ile Gln Met Thr Gln Ser Pro Ser Ser Leu Ser Ala Ser Val Gly
1 5 10 15
Asp Arg Val Thr Ile Thr Cys Arg Ala Ser Gln Asp Ile Arg Asn Tyr
20 25 30
Leu Asn Trp Tyr Gln Gln Lys Pro Gly Lys Ala Pro Lys Leu Leu Ile
35 40 45
Tyr Tyr Thr Ser Arg Leu Glu Ser Gly Val Pro Ser Arg Phe Ser Gly
50 55 60
Ser Gly Ser Gly Thr Asp Tyr Thr Leu Thr Ile Ser Ser Leu Gln Pro
65 70 75 80
Glu Asp Phe Ala Thr Tyr Tyr Cys Gln Gln Gly Asn Thr Leu Pro Trp
85 90 95
Thr Phe Gly Gln Gly Thr Lys Val Glu Ile Lys Arg Thr Val Ala Ala
100 105 110
Pro Ser Val Phe Ile Phe Pro Pro Ser Asp Glu Gln Leu Lys Ser Gly
115 120 125
Thr Ala Ser Val Val Cys Leu Leu Asn Asn Phe Tyr Pro Arg Glu Ala
130 135 140
Lys Val Gln Trp Lys Val Asp Asn Ala Leu Gln Ser Gly Asn Ser Gln
145 150 155 160
Glu Ser Val Thr Glu Gln Asp Ser Lys Asp Ser Thr Tyr Ser Leu Ser
165 170 175
Ser Thr Leu Thr Leu Ser Lys Ala Asp Tyr Glu Lys His Lys Val Tyr
180 185 190
Ala Cys Glu Val Thr His Gln Gly Leu Ser Ser Pro Val Thr Lys Ser
195 200 205
Phe Asn Arg Gly Glu Cys Ser Gly Gly Gly Ser Gly Gly Gly Ser Glu
210 215 220
Gly Gly Gly Ser Glu Gly Gly Gly Ser Glu Gly Gly Gly Ser Glu Gly
225 230 235 240
Gly Gly Ser Gly Gly Gly Ser Gly Glu Val Gln Leu Val Glu Ser Gly
245 250 255
Gly Gly Leu Val Gln Pro Gly Gly Ser Leu Arg Leu Ser Cys Ala Ala
260 265 270
Ser Gly Tyr Ser Phe Thr Gly Tyr Thr Met Asn Trp Val Arg Gln Ala
275 280 285
Pro Gly Lys Gly Leu Glu Trp Val Ala Leu Ile Asn Pro Tyr Lys Gly
290 295 300
Val Ser Thr Tyr Asn Gln Lys Phe Lys Asp Arg Phe Thr Ile Ser Val
305 310 315 320
Asp Lys Ser Lys Asn Thr Ala Tyr Leu Gln Met Asn Ser Leu Arg Ala
325 330 335
Glu Asp Thr Ala Val Tyr Tyr Cys Ala Arg Ser Gly Tyr Tyr Gly Asp
340 345 350
Ser Asp Trp Tyr Phe Asp Val Trp Gly Gln Gly Thr Leu Val Thr Val
355 360 365
Ser Ser Ala Ser Thr Lys Gly Pro Ser Val Phe Pro Leu Ala Pro Ser
370 375 380
Ser Lys Ser Thr Ser Gly Gly Thr Ala Ala Leu Gly Cys Leu Val Lys
385 390 395 400
Asp Tyr Phe Pro Glu Pro Val Thr Val Ser Trp Asn Ser Gly Ala Leu
405 410 415
Thr Ser Gly Val His Thr Phe Pro Ala Val Leu Gln Ser Ser Gly Leu
420 425 430
Tyr Ser Leu Ser Ser Val Val Thr Val Pro Ser Ser Ser Leu Gly Thr
435 440 445
Gln Thr Tyr Ile Cys Asn Val Asn His Lys Pro Ser Asn Thr Lys Val
450 455 460
Asp Lys Lys Val Glu Pro Lys Ser Cys Asp Gly Gly Gly Gly Ser Gly
465 470 475 480
Gly Gly Gly Ser Glu Val Gln Leu Gln Glu Ser Gly Pro Gly Leu Val
485 490 495
Lys Pro Ser Gln Ser Leu Ser Leu Thr Cys Ser Val Thr Gly Tyr Ser
500 505 510
Ile Thr Ser Gly Tyr Tyr Trp Asn Trp Ile Arg Gln Phe Pro Gly Asn
515 520 525
Lys Leu Glu Trp Met Gly Tyr Ile Thr Tyr Asp Gly Ser Asn Asn Tyr
530 535 540
Asn Pro Ser Leu Lys Asn Arg Ile Ser Ile Thr Arg Asp Thr Ser Lys
545 550 555 560
Asn Gln Phe Phe Leu Lys Leu Asn Ser Val Thr Thr Glu Asp Thr Ala
565 570 575
Thr Tyr Tyr Cys Ala Asp Phe Asp Tyr Trp Gly Gln Gly Thr Thr Leu
580 585 590
Thr Val Ser Ser Ala Ser Thr Lys Gly Pro Ser Val Phe Pro Leu Ala
595 600 605
Pro Ser Ser Lys Ser Thr Ser Gly Gly Thr Ala Ala Leu Gly Cys Leu
610 615 620
Val Lys Asp Tyr Phe Pro Glu Pro Val Thr Val Ser Trp Asn Ser Gly
625 630 635 640
Ala Leu Thr Ser Gly Val His Thr Phe Pro Ala Val Leu Gln Ser Ser
645 650 655
Gly Leu Tyr Ser Leu Ser Ser Val Val Thr Val Pro Ser Ser Ser Leu
660 665 670
Gly Thr Gln Thr Tyr Ile Cys Asn Val Asn His Lys Pro Ser Asn Thr
675 680 685
Lys Val Asp Lys Lys Val Glu Pro Lys Ser Cys Asp Lys Thr His Thr
690 695 700
Cys Pro Pro Cys Pro Ala Pro Glu Ala Ala Gly Gly Pro Ser Val Phe
705 710 715 720
Leu Phe Pro Pro Lys Pro Lys Asp Thr Leu Met Ile Ser Arg Thr Pro
725 730 735
Glu Val Thr Cys Val Val Val Asp Val Ser His Glu Asp Pro Glu Val
740 745 750
Lys Phe Asn Trp Tyr Val Asp Gly Val Glu Val His Asn Ala Lys Thr
755 760 765
Lys Pro Arg Glu Glu Gln Tyr Asp Ser Thr Tyr Arg Val Val Ser Val
770 775 780
Leu Thr Val Leu His Gln Asp Trp Leu Asn Gly Lys Glu Tyr Lys Cys
785 790 795 800
Lys Val Ser Asn Lys Ala Leu Gly Ala Pro Ile Glu Lys Thr Ile Ser
805 810 815
Lys Ala Lys Gly Gln Pro Arg Glu Pro Gln Val Tyr Thr Leu Pro Pro
820 825 830
Cys Arg Asp Glu Leu Thr Lys Asn Gln Val Ser Leu Trp Cys Leu Val
835 840 845
Lys Gly Phe Tyr Pro Ser Asp Ile Ala Val Glu Trp Glu Ser Asn Gly
850 855 860
Gln Pro Glu Asn Asn Tyr Lys Thr Thr Pro Pro Val Leu Asp Ser Asp
865 870 875 880
Gly Ser Phe Phe Leu Tyr Ser Lys Leu Thr Val Asp Lys Ser Arg Trp
885 890 895
Gln Gln Gly Asn Val Phe Ser Cys Ser Val Met His Glu Ala Leu His
900 905 910
Asn His Tyr Thr Gln Lys Ser Leu Ser Leu Ser Pro Gly Lys
915 920 925

SEQ ID NO: 26
<212> DNA
<223> V9 (scFab)-LC007 (VH-CH1)-Fc(knob) P329G LALA N297D
gacatccaga tgacccagag cccctctagc ctgagcgcca gcgtgggcga cagagtgacc 60
atcacctgtc gggccagcca ggacatcaga aactacctga actggtatca gcagaagccc 120
ggcaaggccc ccaagctgct gatctactac acctctagac tggaaagcgg cgtgcccagc 180
cggtttagcg gcagcggctc cggcaccgac tacaccctga ccatcagcag cctgcagccc 240
gaggacttcg ccacctacta ctgccagcag ggcaacacac tcccctggac cttcggccag 300
ggcaccaagg tggagatcaa gcgtacggtg gccgctccca gcgtgttcat cttccccccc 360
agcgacgagc agctgaagtc cggcaccgcc agcgtcgtgt gcctgctgaa caacttctac 420
ccccgggagg ccaaggtgca gtggaaggtg gacaacgccc tgcagagcgg caacagccag 480
gaaagcgtca ccgagcagga cagcaaggac tccacctaca gcctgagcag caccctgacc 540
ctgagcaagg ccgactacga gaagcacaag gtgtacgcct gcgaagtgac ccaccagggc 600
ctgtccagcc ccgtgaccaa gagcttcaac cggggcgagt gcagcggcgg aggctctgga 660
ggcggctctg aaggcggagg aagtgagggc ggaggctcag aaggcggcgg aagcgaaggt 720
ggcggctctg gcggcggatc cggcgaggtg cagctggtcg agtccggcgg aggcctggtg 780
cagcctggcg gcagcctgag actgagctgc gccgccagcg gctacagctt caccggctac 840
accatgaact gggtccggca ggctcctggc aagggcctcg aatgggtggc cctgatcaac 900
ccctacaagg gcgtgagcac ctacaaccag aagttcaagg accggttcac catcagcgtg 960
gacaagagca agaacaccgc ctatctgcag atgaacagcc tgcgggccga ggacaccgcc 1020
gtgtactact gcgccagaag cggctactac ggcgacagcg actggtactt cgacgtgtgg 1080
ggccagggca cactggtcac cgtgtccagc gctagcacca agggcccctc cgtgttcccc 1140
ctggccccca gcagcaagag caccagcggc ggcacagccg ccctcggctg cctggtcaag 1200
gactacttcc ccgagcccgt gaccgtgtcc tggaacagcg gagccctgac ctccggcgtg 1260
cacaccttcc ccgccgtgct gcagagcagc ggcctgtaca gcctgtccag cgtggtcacc 1320
gtgccctcca gcagcctggg cacccagacc tacatctgca acgtgaacca caagcccagc 1380
aataccaagg tggacaagaa ggtggagccc aagagctgcg acggcggtgg tggctccgga 1440
ggcggtggat ctgaagtgca gctgcaggaa agcggccctg gcctggtcaa gcccagccag 1500
agcctgagcc tgacctgtag cgtgaccggc tactccatca cctccggcta ctactggaat 1560
tggattcggc agttccccgg caacaagctg gaatggatgg gctacatcac ctacgacggc 1620
agcaacaact acaaccccag cctgaagaac cggatcagca tcacccggga caccagcaag 1680
aaccagttct tcctgaagtt gaattctgtg actactgagg acacagctac atattactgt 1740
gcggactttg actactgggg ccaaggcacc actctcacag tctcctcagc tagcaccaag 1800
ggcccatcgg tcttccccct ggcaccctcc tccaagagca cctctggggg cacagcggcc 1860
ctgggctgcc tggtcaagga ctacttcccc gaaccggtga cggtgtcgtg gaactcaggc 1920
gccctgacca gcggcgtgca caccttcccg gctgtcctac agtcctcagg actctactcc 1980
ctcagcagcg tggtgaccgt gccctccagc agcttgggca cccagaccta catctgcaac 2040
gtgaatcaca agcccagcaa caccaaggtg gacaagaaag ttgagcccaa atcttgtgac 2100
aaaactcaca catgcccacc gtgcccagca cctgaagctg cagggggacc gtcagtcttc 2160
ctcttccccc caaaacccaa ggacaccctc atgatctccc ggacccctga ggtcacatgc 2220
gtggtggtgg acgtgagcca cgaagaccct gaggtcaagt tcaactggta cgtggacggc 2280
gtggaggtgc ataatgccaa gacaaagccg cgggaggagc agtacgacag cacgtaccgt 2340
gtggtcagcg tcctcaccgt cctgcaccag gactggctga atggcaagga gtacaagtgc 2400
aaggtctcca acaaagccct cggcgccccc atcgagaaaa ccatctccaa agccaaaggg 2460
cagccccgag aaccacaggt gtacaccctg cccccatgcc gggatgagct gaccaagaac 2520
caggtcagcc tgtggtgcct ggtcaaaggc ttctatccca gcgacatcgc cgtggagtgg 2580
gagagcaatg ggcagccgga gaacaactac aagaccacgc ctcccgtgct ggactccgac 2640
ggctccttct tcctctacag caagctcacc gtggacaaga gcaggtggca gcaggggaac 2700
gtcttctcat gctccgtgat gcatgaggct ctgcacaacc actacacgca gaagagcctc 2760
tccctgtctc cgggtaaatg a 2781

SEQ ID NO: 27
<212> PRT
<223> LC007 (VH-CH1)-Fc(hole) P329G LALA N297D
Glu Val Gln Leu Gln Glu Ser Gly Pro Gly Leu Val Lys Pro Ser Gln
1 5 10 15
Ser Leu Ser Leu Thr Cys Ser Val Thr Gly Tyr Ser Ile Thr Ser Gly
20 25 30
Tyr Tyr Trp Asn Trp Ile Arg Gln Phe Pro Gly Asn Lys Leu Glu Trp
35 40 45
Met Gly Tyr Ile Thr Tyr Asp Gly Ser Asn Asn Tyr Asn Pro Ser Leu
50 55 60
Lys Asn Arg Ile Ser Ile Thr Arg Asp Thr Ser Lys Asn Gln Phe Phe
65 70 75 80
Leu Lys Leu Asn Ser Val Thr Thr Glu Asp Thr Ala Thr Tyr Tyr Cys
85 90 95
Ala Asp Phe Asp Tyr Trp Gly Gln Gly Thr Thr Leu Thr Val Ser Ser
100 105 110
Ala Ser Thr Lys Gly Pro Ser Val Phe Pro Leu Ala Pro Ser Ser Lys
115 120 125
Ser Thr Ser Gly Gly Thr Ala Ala Leu Gly Cys Leu Val Lys Asp Tyr
130 135 140
Phe Pro Glu Pro Val Thr Val Ser Trp Asn Ser Gly Ala Leu Thr Ser
145 150 155 160
Gly Val His Thr Phe Pro Ala Val Leu Gln Ser Ser Gly Leu Tyr Ser
165 170 175
Leu Ser Ser Val Val Thr Val Pro Ser Ser Ser Leu Gly Thr Gln Thr
180 185 190
Tyr Ile Cys Asn Val Asn His Lys Pro Ser Asn Thr Lys Val Asp Lys
195 200 205
Lys Val Glu Pro Lys Ser Cys Asp Lys Thr His Thr Cys Pro Pro Cys
210 215 220
Pro Ala Pro Glu Ala Ala Gly Gly Pro Ser Val Phe Leu Phe Pro Pro
225 230 235 240
Lys Pro Lys Asp Thr Leu Met Ile Ser Arg Thr Pro Glu Val Thr Cys
245 250 255
Val Val Val Asp Val Ser His Glu Asp Pro Glu Val Lys Phe Asn Trp
260 265 270
Tyr Val Asp Gly Val Glu Val His Asn Ala Lys Thr Lys Pro Arg Glu
275 280 285
Glu Gln Tyr Asp Ser Thr Tyr Arg Val Val Ser Val Leu Thr Val Leu
290 295 300
His Gln Asp Trp Leu Asn Gly Lys Glu Tyr Lys Cys Lys Val Ser Asn
305 v 310 315 320
Lys Ala Leu Gly Ala Pro Ile Glu Lys Thr Ile Ser Lys Ala Lys Gly
325 330 335
Gln Pro Arg Glu Pro Gln Val Cys Thr Leu Pro Pro Ser Arg Asp Glu
340 345 350
Leu Thr Lys Asn Gln Val Ser Leu Ser Cys Ala Val Lys Gly Phe Tyr
355 360 365
Pro Ser Asp Ile Ala Val Glu Trp Glu Ser Asn Gly Gln Pro Glu Asn
370 375 380
Asn Tyr Lys Thr Thr Pro Pro Val Leu Asp Ser Asp Gly Ser Phe Phe
385 390 395 400
Leu Val Ser Lys Leu Thr Val Asp Lys Ser Arg Trp Gln Gln Gly Asn
405 410 415
Val Phe Ser Cys Ser Val Met His Glu Ala Leu His Asn His Tyr Thr
420 425 430
Gln Lys Ser Leu Ser Leu Ser Pro Gly Lys
435 440

SEQ ID NO: 28
<212> DNA
<223> LC007 (VH-CH1)-Fc(hole) P329G LALA N297D
gaggtccagc tgcaggagtc aggacctggc ctcgtgaaac cttctcagtc tctgtctctc 60
acctgctctg tcactggcta ctccatcacc agtggttatt actggaactg gatccggcag 120
tttccaggaa acaagctgga atggatgggc tacataacct acgacggtag caataactac 180
aacccatctc tcaaaaatcg aatctccatc actcgtgaca catctaagaa ccagtttttc 240
ctgaagttga attctgtgac tactgaggac acagctacat attactgtgc ggactttgac 300
tactggggcc aaggcaccac tctcacagtc tcctcagcta gcaccaaggg cccatcggtc 360
ttccccctgg caccctcctc caagagcacc tctgggggca cagcggccct gggctgcctg 420
gtcaaggact acttccccga accggtgacg gtgtcgtgga actcaggcgc cctgaccagc 480
ggcgtgcaca ccttcccggc tgtcctacag tcctcaggac tctactccct cagcagcgtg 540
gtgaccgtgc cctccagcag cttgggcacc cagacctaca tctgcaacgt gaatcacaag 600
cccagcaaca ccaaggtgga caagaaagtt gagcccaaat cttgtgacaa aactcacaca 660
tgcccaccgt gcccagcacc tgaagctgca gggggaccgt cagtcttcct cttcccccca 720
aaacccaagg acaccctcat gatctcccgg acccctgagg tcacatgcgt ggtggtggac 780
gtgagccacg aagaccctga ggtcaagttc aactggtacg tggacggcgt ggaggtgcat 840
aatgccaaga caaagccgcg ggaggagcag tacgacagca cgtaccgtgt ggtcagcgtc 900
ctcaccgtcc tgcaccagga ctggctgaat ggcaaggagt acaagtgcaa ggtctccaac 960
aaagccctcg gcgcccccat cgagaaaacc atctccaaag ccaaagggca gccccgagaa 1020
ccacaggtgt gcaccctgcc cccatcccgg gatgagctga ccaagaacca ggtcagcctc 1080
tcgtgcgcag tcaaaggctt ctatcccagc gacatcgccg tggagtggga gagcaatggg 1140
cagccggaga acaactacaa gaccacgcct cccgtgctgg actccgacgg ctccttcttc 1200
ctcgtgagca agctcaccgt ggacaagagc aggtggcagc aggggaacgt cttctcatgc 1260
tccgtgatgc atgaggctct gcacaaccac tacacgcaga agagcctctc cctgtctccg 1320
ggtaaatga 1329

SEQ ID NO: 29
<212> PRT
<223> V9 (VL-CH1)-LC007 (VH-CH1)-Fc(hole) P329G LALA
Asp Ile Gln Met Thr Gln Ser Pro Ser Ser Leu Ser Ala Ser Val Gly
1 5 10 15
Asp Arg Val Thr Ile Thr Cys Arg Ala Ser Gln Asp Ile Arg Asn Tyr
20 25 30
Leu Asn Trp Tyr Gln Gln Lys Pro Gly Lys Ala Pro Lys Leu Leu Ile
35 40 45
Tyr Tyr Thr Ser Arg Leu Glu Ser Gly Val Pro Ser Arg Phe Ser Gly
50 55 60
Ser Gly Ser Gly Thr Asp Tyr Thr Leu Thr Ile Ser Ser Leu Gln Pro
65 70 75 80
Glu Asp Phe Ala Thr Tyr Tyr Cys Gln Gln Gly Asn Thr Leu Pro Trp
85 90 95
Thr Phe Gly Gln Gly Thr Lys Val Glu Ile Lys Ser Ser Ala Ser Thr
100 105 110
Lys Gly Pro Ser Val Phe Pro Leu Ala Pro Ser Ser Lys Ser Thr Ser
115 120 125
Gly Gly Thr Ala Ala Leu Gly Cys Leu Val Lys Asp Tyr Phe Pro Glu
130 135 140
Pro Val Thr Val Ser Trp Asn Ser Gly Ala Leu Thr Ser Gly Val His
145 150 155 160
Thr Phe Pro Ala Val Leu Gln Ser Ser Gly Leu Tyr Ser Leu Ser Ser
165 170 175
Val Val Thr Val Pro Ser Ser Ser Leu Gly Thr Gln Thr Tyr Ile Cys
180 185 190
Asn Val Asn His Lys Pro Ser Asn Thr Lys Val Asp Lys Lys Val Glu
195 200 205
Pro Lys Ser Cys Gly Gly Gly Gly Ser Gly Gly Gly Gly Ser Glu Val
210 215 220
Gln Leu Gln Glu Ser Gly Pro Gly Leu Val Lys Pro Ser Gln Ser Leu
225 230 235 240
Ser Leu Thr Cys Ser Val Thr Gly Tyr Ser Ile Thr Ser Gly Tyr Tyr
245 250 255
Trp Asn Trp Ile Arg Gln Phe Pro Gly Asn Lys Leu Glu Trp Met Gly
260 265 270
Tyr Ile Thr Tyr Asp Gly Ser Asn Asn Tyr Asn Pro Ser Leu Lys Asn
275 280 285
Arg Ile Ser Ile Thr Arg Asp Thr Ser Lys Asn Gln Phe Phe Leu Lys
290 295 300
Leu Asn Ser Val Thr Thr Glu Asp Thr Ala Thr Tyr Tyr Cys Ala Asp
305 310 315 320
Phe Asp Tyr Trp Gly Gln Gly Thr Thr Leu Thr Val Ser Ser Ala Ser
325 330 335
Thr Lys Gly Pro Ser Val Phe Pro Leu Ala Pro Ser Ser Lys Ser Thr
340 345 350
Ser Gly Gly Thr Ala Ala Leu Gly Cys Leu Val Lys Asp Tyr Phe Pro
355 360 365
Glu Pro Val Thr Val Ser Trp Asn Ser Gly Ala Leu Thr Ser Gly Val
370 375 380
His Thr Phe Pro Ala Val Leu Gln Ser Ser Gly Leu Tyr Ser Leu Ser
385 390 395 400
Ser Val Val Thr Val Pro Ser Ser Ser Leu Gly Thr Gln Thr Tyr Ile
405 410 415
Cys Asn Val Asn His Lys Pro Ser Asn Thr Lys Val Asp Lys Lys Val
420 425 430
Glu Pro Lys Ser Cys Asp Lys Thr His Thr Cys Pro Pro Cys Pro Ala
435 440 445
Pro Glu Ala Ala Gly Gly Pro Ser Val Phe Leu Phe Pro Pro Lys Pro
450 455 460
Lys Asp Thr Leu Met Ile Ser Arg Thr Pro Glu Val Thr Cys Val Val
465 470 475 480
Val Asp Val Ser His Glu Asp Pro Glu Val Lys Phe Asn Trp Tyr Val
485 490 495
Asp Gly Val Glu Val His Asn Ala Lys Thr Lys Pro Arg Glu Glu Gln
500 505 510
Tyr Asn Ser Thr Tyr Arg Val Val Ser Val Leu Thr Val Leu His Gln
515 520 525
Asp Trp Leu Asn Gly Lys Glu Tyr Lys Cys Lys Val Ser Asn Lys Ala
530 535 540
Leu Gly Ala Pro Ile Glu Lys Thr Ile Ser Lys Ala Lys Gly Gln Pro
545 550 555 560
Arg Glu Pro Gln Val Cys Thr Leu Pro Pro Ser Arg Asp Glu Leu Thr
565 570 575
Lys Asn Gln Val Ser Leu Ser Cys Ala Val Lys Gly Phe Tyr Pro Ser
580 585 590
Asp Ile Ala Val Glu Trp Glu Ser Asn Gly Gln Pro Glu Asn Asn Tyr
595 600 605
Lys Thr Thr Pro Pro Val Leu Asp Ser Asp Gly Ser Phe Phe Leu Val
610 615 620
Ser Lys Leu Thr Val Asp Lys Ser Arg Trp Gln Gln Gly Asn Val Phe
625 630 635 640
Ser Cys Ser Val Met His Glu Ala Leu His Asn His Tyr Thr Gln Lys
645 650 655
Ser Leu Ser Leu Ser Pro Gly Lys
660

SEQ ID NO: 30
<212> DNA
<223> V9 (VL-CH1)-LC007 (VH-CH1)-Fc(hole) P329G LALA
gatatccaga tgacccagag ccccagctct ctgagcgcca gcgtgggcga cagagtgacc 60
atcacctgtc gggccagcca ggacatcaga aactacctga actggtatca gcagaagccc 120
ggcaaggccc ccaagctgct gatctactac accagcagac tggaaagcgg cgtgccctcc 180
agattttccg gcagcggctc cggcaccgac tacaccctga ccatcagcag cctgcagccc 240
gaggatttcg ccacatatta ctgccagcag ggcaataccc tgccctggac cttcggacag 300
ggcacaaaag tggaaatcaa gagcagcgct tccaccaaag gcccttccgt gtttcctctg 360
gctcctagct ccaagtccac ctctggaggc accgctgctc tcggatgcct cgtgaaggat 420
tattttcctg agcctgtgac agtgtcctgg aatagcggag cactgacctc tggagtgcat 480
actttccccg ctgtgctgca gtcctctgga ctgtacagcc tgagcagcgt ggtgacagtg 540
cccagcagca gcctgggcac ccagacctac atctgcaacg tgaaccacaa gcccagcaac 600
accaaggtgg acaagaaggt ggaacccaag tcttgtggcg gaggcggatc cggcggaggg 660
ggatctgagg tgcagctgca ggaaagcggc cctggcctgg tgaaacccag ccagagcctg 720
agcctgacct gcagcgtgac cggctacagc atcaccagcg gctactactg gaactggatc 780
agacagttcc ccggcaacaa gctggaatgg atgggctaca tcacctacga cggcagcaac 840
aactacaacc ccagcctgaa gaacagaatc agcatcaccc gggacaccag caagaaccag 900
ttcttcctga agctgaacag cgtgaccacc gaggacaccg ccacctacta ctgcgccgac 960
ttcgactact ggggccaggg caccaccctg accgtgtcct ccgcctctac caagggcccc 1020
agcgtgttcc ccctggcacc cagcagcaag agcacatctg gcggaacagc cgctctgggc 1080
tgtctggtga aagactactt ccccgagccc gtgaccgtgt cttggaactc tggcgccctg 1140
accagcggcg tgcacacctt tccagccgtg ctgcagagca gcggcctgta ctccctgtcc 1200
tccgtggtca ccgtgccctc tagctccctg ggaacacaga catatatctg taatgtcaat 1260
cacaagcctt ccaacaccaa agtcgataag aaagtcgagc ccaagagctg cgacaaaact 1320
cacacatgcc caccgtgccc agcacctgaa gctgcagggg gaccgtcagt cttcctcttc 1380
cccccaaaac ccaaggacac cctcatgatc tcccggaccc ctgaggtcac atgcgtggtg 1440
gtggacgtga gccacgaaga ccctgaggtc aagttcaact ggtacgtgga cggcgtggag 1500
gtgcataatg ccaagacaaa gccgcgggag gagcagtaca acagcacgta ccgtgtggtc 1560
agcgtcctca ccgtcctgca ccaggactgg ctgaatggca aggagtacaa gtgcaaggtc 1620
tccaacaaag ccctcggcgc ccccatcgag aaaaccatct ccaaagccaa agggcagccc 1680
cgagaaccac aggtgtgcac cctgccccca tcccgggatg agctgaccaa gaaccaggtc 1740
agcctctcgt gcgcagtcaa aggcttctat cccagcgaca tcgccgtgga gtgggagagc 1800
aatgggcagc cggagaacaa ctacaagacc acgcctcccg tgctggactc cgacggctcc 1860
ttcttcctcg tgagcaagct caccgtggac aagagcaggt ggcagcaggg gaacgtcttc 1920
tcatgctccg tgatgcatga ggctctgcac aaccactaca cgcagaagag cctctccctg 1980
tctccgggta aatga 1995

SEQ ID NO: 31
<212> PRT
<223> Fc(knob) wt
Asp Lys Thr His Thr Cys Pro Pro Cys Pro Ala Pro Glu Leu Leu Gly
1 5 10 15
Gly Pro Ser Val Phe Leu Phe Pro Pro Lys Pro Lys Asp Thr Leu Met
20 25 30
Ile Ser Arg Thr Pro Glu Val Thr Cys Val Val Val Asp Val Ser His
35 40 45
Glu Asp Pro Glu Val Lys Phe Asn Trp Tyr Val Asp Gly Val Glu Val
50 55 60
His Asn Ala Lys Thr Lys Pro Arg Glu Glu Gln Tyr Asn Ser Thr Tyr
65 70 75 80
Arg Val Val Ser Val Leu Thr Val Leu His Gln Asp Trp Leu Asn Gly
85 90 95
Lys Glu Tyr Lys Cys Lys Val Ser Asn Lys Ala Leu Pro Ala Pro Ile
100 105 110
Glu Lys Thr Ile Ser Lys Ala Lys Gly Gln Pro Arg Glu Pro Gln Val
115 120 125
Tyr Thr Leu Pro Pro Cys Arg Asp Glu Leu Thr Lys Asn Gln Val Ser
130 135 140
Leu Trp Cys Leu Val Lys Gly Phe Tyr Pro Ser Asp Ile Ala Val Glu
145 150 155 160
Trp Glu Ser Asn Gly Gln Pro Glu Asn Asn Tyr Lys Thr Thr Pro Pro
165 170 175
Val Leu Asp Ser Asp Gly Ser Phe Phe Leu Tyr Ser Lys Leu Thr Val
180 185 190
Asp Lys Ser Arg Trp Gln Gln Gly Asn Val Phe Ser Cys Ser Val Met
195 200 205
His Glu Ala Leu His Asn His Tyr Thr Gln Lys Ser Leu Ser Leu Ser
210 215 220
Pro Gly Lys Ser Gly
225

SEQ ID NO: 32
<212> DNA
<223> Fc(knob) wt
gacaaaactc acacatgccc accgtgccca gcacctgaac tcctgggggg accgtcagtc 60
ttcctcttcc ccccaaaacc caaggacacc ctcatgatct cccggacccc tgaggtcaca 120
tgcgtggtgg tggacgtgag ccacgaagac cctgaggtca agttcaactg gtacgtggac 180
ggcgtggagg tgcataatgc caagacaaag ccgcgggagg agcagtacaa cagcacgtac 240
cgtgtggtca gcgtcctcac cgtcctgcac caggactggc tgaatggcaa ggagtacaag 300
tgcaaggtct ccaacaaagc cctcccagcc cccatcgaga aaaccatctc caaagccaaa 360
gggcagcccc gagaaccaca ggtgtacacc ctgcccccat gccgggatga gctgaccaag 420
aaccaggtca gcctgtggtg cctggtcaaa ggcttctatc ccagcgacat cgccgtggag 480
tgggagagca atgggcagcc ggagaacaac tacaagacca cgcctcccgt gctggactcc 540
gacggctcct tcttcctcta cagcaagctc accgtggaca agagcaggtg gcagcagggg 600
aacgtcttct catgctccgt gatgcatgag gctctgcaca accactacac gcagaagagc 660
ctctccctgt ctccgggtaa atccggatga 690

SEQ ID NO: 33
<212> PRT
<223> V9 (VH-CL)
Glu Val Gln Leu Val Glu Ser Gly Gly Gly Leu Val Gln Pro Gly Gly
1 5 10 15
Ser Leu Arg Leu Ser Cys Ala Ala Ser Gly Tyr Ser Phe Thr Gly Tyr
20 25 30
Thr Met Asn Trp Val Arg Gln Ala Pro Gly Lys Gly Leu Glu Trp Val
35 40 45
Ala Leu Ile Asn Pro Tyr Lys Gly Val Ser Thr Tyr Asn Gln Lys Phe
50 55 60
Lys Asp Arg Phe Thr Ile Ser Val Asp Lys Ser Lys Asn Thr Ala Tyr
65 70 75 80
Leu Gln Met Asn Ser Leu Arg Ala Glu Asp Thr Ala Val Tyr Tyr Cys
85 90 95
Ala Arg Ser Gly Tyr Tyr Gly Asp Ser Asp Trp Tyr Phe Asp Val Trp
100 105 110
Gly Gln Gly Thr Leu Val Thr Val Ser Ser Ala Ser Val Ala Ala Pro
115 120 125
Ser Val Phe Ile Phe Pro Pro Ser Asp Glu Gln Leu Lys Ser Gly Thr
130 135 140
Ala Ser Val Val Cys Leu Leu Asn Asn Phe Tyr Pro Arg Glu Ala Lys
145 150 155 160
Val Gln Trp Lys Val Asp Asn Ala Leu Gln Ser Gly Asn Ser Gln Glu
165 170 175
Ser Val Thr Glu Gln Asp Ser Lys Asp Ser Thr Tyr Ser Leu Ser Ser
180 185 190
Thr Leu Thr Leu Ser Lys Ala Asp Tyr Glu Lys His Lys Val Tyr Ala
195 200 205
Cys Glu Val Thr His Gln Gly Leu Ser Ser Pro Val Thr Lys Ser Phe
210 215 220
Asn Arg Gly Glu Cys
225

SEQ ID NO: 34
<212> DNA
<223> V9 (VH-CL)
gaggtgcagc tggtcgagag cggaggcggc ctggtgcagc ctggcggcag cctgagactg 60
agctgcgccg ccagcggcta cagcttcacc ggctacacca tgaactgggt ccggcaggca 120
cctggcaagg gactggaatg ggtggccctg atcaacccct acaagggcgt gagcacctac 180
aaccagaagt tcaaggaccg gttcaccatc agcgtggaca agagcaagaa caccgcctat 240
ctgcagatga acagcctgcg ggccgaggac accgccgtgt actactgcgc cagaagcggc 300
tactacggcg acagcgactg gtacttcgac gtgtggggcc agggcaccct cgtgaccgtg 360
tctagcgcta gcgtggctgc accatctgtc ttcatcttcc cgccatctga tgagcagttg 420
aaatctggaa ctgcctctgt tgtgtgcctg ctgaataact tctatcccag agaggccaaa 480
gtacagtgga aggtggataa cgccctccaa tcgggtaact cccaggagag tgtcacagag 540
caggacagca aggacagcac ctacagcctc agcagcaccc tgacgctgag caaagcagac 600
tacgagaaac acaaagtcta cgcctgcgaa gtcacccatc agggcctgag ctcgcccgtc 660
acaaagagct tcaacagggg agagtgttga 690

SEQ ID NO: 35
<212> PRT
<223> FN18 (VL-CH1)-LC007 (VH-CH1)-Fc(hole) P329G LALA
Asp Ile Val Met Ser Gln Ser Pro Ser Ser Leu Ala Val Ser Val Gly
1 5 10 15
Glu Lys Val Thr Met Ser Cys Lys Ser Ser Gln Ser Leu Leu Tyr Ser
20 25 30
Ser Asn Gln Lys Asn Tyr Leu Ala Trp Tyr Gln Gln Lys Pro Gly Gln
35 40 45
Ser Pro Lys Leu Leu Ile Asn Trp Ala Ser Thr Arg Glu Ser Gly Val
50 55 60
Pro Asp Arg Phe Thr Gly Ser Gly Ser Arg Thr Asp Phe Thr Leu Thr
65 70 75 80
Ile Ser Ser Val Lys Ala Glu Asp Leu Ala Val Tyr Phe Cys Gln Gln
85 90 95
Phe Tyr Ser Tyr Pro Pro Thr Phe Gly Gly Gly Thr Lys Leu Glu Ile
100 105 110
Lys Ser Ser Ala Ser Thr Lys Gly Pro Ser Val Phe Pro Leu Ala Pro
115 120 125
Ser Ser Lys Ser Thr Ser Gly Gly Thr Ala Ala Leu Gly Cys Leu Val
130 135 140
Lys Asp Tyr Phe Pro Glu Pro Val Thr Val Ser Trp Asn Ser Gly Ala
145 150 155 160
Leu Thr Ser Gly Val His Thr Phe Pro Ala Val Leu Gln Ser Ser Gly
165 170 175
Leu Tyr Ser Leu Ser Ser Val Val Thr Val Pro Ser Ser Ser Leu Gly
180 185 190
Thr Gln Thr Tyr Ile Cys Asn Val Asn His Lys Pro Ser Asn Thr Lys
195 200 205
Val Asp Lys Lys Val Glu Pro Lys Ser Cys Gly Gly Gly Gly Ser Gly
210 215 220
Gly Gly Gly Ser Glu Val Gln Leu Gln Glu Ser Gly Pro Gly Leu Val
225 230 235 240
Lys Pro Ser Gln Ser Leu Ser Leu Thr Cys Ser Val Thr Gly Tyr Ser
245 250 255
Ile Thr Ser Gly Tyr Tyr Trp Asn Trp Ile Arg Gln Phe Pro Gly Asn
260 265 270
Lys Leu Glu Trp Met Gly Tyr Ile Thr Tyr Asp Gly Ser Asn Asn Tyr
275 280 285
Asn Pro Ser Leu Lys Asn Arg Ile Ser Ile Thr Arg Asp Thr Ser Lys
290 295 300
Asn Gln Phe Phe Leu Lys Leu Asn Ser Val Thr Thr Glu Asp Thr Ala
305 310 315 320
Thr Tyr Tyr Cys Ala Asp Phe Asp Tyr Trp Gly Gln Gly Thr Thr Leu
325 330 335
Thr Val Ser Ser Ala Ser Thr Lys Gly Pro Ser Val Phe Pro Leu Ala
340 345 350
Pro Ser Ser Lys Ser Thr Ser Gly Gly Thr Ala Ala Leu Gly Cys Leu
355 360 365
Val Lys Asp Tyr Phe Pro Glu Pro Val Thr Val Ser Trp Asn Ser Gly
370 375 380
Ala Leu Thr Ser Gly Val His Thr Phe Pro Ala Val Leu Gln Ser Ser
385 390 395 400
Gly Leu Tyr Ser Leu Ser Ser Val Val Thr Val Pro Ser Ser Ser Leu
405 410 415
Gly Thr Gln Thr Tyr Ile Cys Asn Val Asn His Lys Pro Ser Asn Thr
420 425 430
Lys Val Asp Lys Lys Val Glu Pro Lys Ser Cys Asp Lys Thr His Thr
435 440 445
Cys Pro Pro Cys Pro Ala Pro Glu Ala Ala Gly Gly Pro Ser Val Phe
450 455 460
Leu Phe Pro Pro Lys Pro Lys Asp Thr Leu Met Ile Ser Arg Thr Pro
465 470 475 480
Glu Val Thr Cys Val Val Val Asp Val Ser His Glu Asp Pro Glu Val
485 490 495
Lys Phe Asn Trp Tyr Val Asp Gly Val Glu Val His Asn Ala Lys Thr
500 505 510
Lys Pro Arg Glu Glu Gln Tyr Asn Ser Thr Tyr Arg Val Val Ser Val
515 520 525
Leu Thr Val Leu His Gln Asp Trp Leu Asn Gly Lys Glu Tyr Lys Cys
530 535 540
Lys Val Ser Asn Lys Ala Leu Gly Ala Pro Ile Glu Lys Thr Ile Ser
545 550 555 560
Lys Ala Lys Gly Gln Pro Arg Glu Pro Gln Val Cys Thr Leu Pro Pro
565 570 575
Ser Arg Asp Glu Leu Thr Lys Asn Gln Val Ser Leu Ser Cys Ala Val
580 585 590
Lys Gly Phe Tyr Pro Ser Asp Ile Ala Val Glu Trp Glu Ser Asn Gly
595 600 605
Gln Pro Glu Asn Asn Tyr Lys Thr Thr Pro Pro Val Leu Asp Ser Asp
610 615 620
Gly Ser Phe Phe Leu Val Ser Lys Leu Thr Val Asp Lys Ser Arg Trp
625 630 635 640
Gln Gln Gly Asn Val Phe Ser Cys Ser Val Met His Glu Ala Leu His
645 650 655
Asn His Tyr Thr Gln Lys Ser Leu Ser Leu Ser Pro Gly Lys
660 665 670

SEQ ID NO: 36
<212> DNA
<223> FN18 (VL-CH1)-LC007 (VH-CH1)-Fc(hole) P329G LALA
gacatcgtga tgagccagag ccccagcagc ctggccgtgt ccgtgggcga gaaagtgacc 60
atgagctgca agagcagcca gagcctgctg tactcctcta accagaagaa ctacctggcc 120
tggtatcagc agaagcccgg ccagtccccc aagctgctga tcaactgggc cagcacccgc 180
gagagcggcg tgcccgatag attcacaggc agcggcagcc ggaccgactt caccctgacc 240
atcagcagcg tgaaggccga ggatctggcc gtgtacttct gccagcagtt ctacagctac 300
ccccccacct tcggcggagg cacgaagctg gaaatcaaga gcagcgcttc caccaaaggc 360
ccttccgtgt ttcctctggc tcctagctcc aagtccacct ctggaggcac cgctgctctc 420
ggatgcctcg tgaaggatta ttttcctgag cctgtgacag tgtcctggaa tagcggagca 480
ctgacctctg gagtgcatac tttccccgct gtgctgcagt cctctggact gtacagcctg 540
agcagcgtgg tgacagtgcc cagcagcagc ctgggcaccc agacctacat ctgcaacgtg 600
aaccacaagc ccagcaacac caaggtggac aagaaggtgg aacccaagtc ttgtggcgga 660
ggcggatccg gcggaggggg atctgaggtg cagctgcagg aaagcggccc tggcctggtg 720
aaacccagcc agagcctgag cctgacctgc agcgtgaccg gctacagcat caccagcggc 780
tactactgga actggatcag acagttcccc ggcaacaagc tggaatggat gggctacatc 840
acctacgacg gcagcaacaa ctacaacccc agcctgaaga acagaatcag catcacccgg 900
gacaccagca agaaccagtt cttcctgaag ctgaacagcg tgaccaccga ggacaccgcc 960
acctactact gcgccgactt cgactactgg ggccagggca ccaccctgac cgtgtcctcc 1020
gcctctacca agggccccag cgtgttcccc ctggcaccca gcagcaagag cacatctggc 1080
ggaacagccg ctctgggctg tctggtgaaa gactacttcc ccgagcccgt gaccgtgtct 1140
tggaactctg gcgccctgac cagcggcgtg cacacctttc cagccgtgct gcagagcagc 1200
ggcctgtact ccctgtcctc cgtggtcacc gtgccctcta gctccctggg aacacagaca 1260
tatatctgta atgtcaatca caagccttcc aacaccaaag tcgataagaa agtcgagccc 1320
aagagctgcg acaaaactca cacatgccca ccgtgcccag cacctgaagc tgcaggggga 1380
ccgtcagtct tcctcttccc cccaaaaccc aaggacaccc tcatgatctc ccggacccct 1440
gaggtcacat gcgtggtggt ggacgtgagc cacgaagacc ctgaggtcaa gttcaactgg 1500
tacgtggacg gcgtggaggt gcataatgcc aagacaaagc cgcgggagga gcagtacaac 1560
agcacgtacc gtgtggtcag cgtcctcacc gtcctgcacc aggactggct gaatggcaag 1620
gagtacaagt gcaaggtctc caacaaagcc ctcggcgccc ccatcgagaa aaccatctcc 1680
aaagccaaag ggcagccccg agaaccacag gtgtgcaccc tgcccccatc ccgggatgag 1740
ctgaccaaga accaggtcag cctctcgtgc gcagtcaaag gcttctatcc cagcgacatc 1800
gccgtggagt gggagagcaa tgggcagccg gagaacaact acaagaccac gcctcccgtg 1860
ctggactccg acggctcctt cttcctcgtg agcaagctca ccgtggacaa gagcaggtgg 1920
cagcagggga acgtcttctc atgctccgtg atgcatgagg ctctgcacaa ccactacacg 1980
cagaagagcc tctccctgtc tccgggtaaa tga 2013

SEQ ID NO: 37
<212> PRT
<223> FN18 (VH-CL)
Gln Val Gln Leu Gln Gln Ser Glu Ala Glu Leu Ala Arg Pro Gly Ala
1 5 10 15
Ser Val Lys Met Ser Cys Lys Ala Ser Gly Tyr Thr Phe Thr Asp Tyr
20 25 30
Thr Ile His Trp Leu Lys Gln Arg Pro Gly Gln Gly Leu Asp Trp Ile
35 40 45
Gly Tyr Phe Asn Pro Ser Ser Glu Ser Thr Glu Tyr Asn Arg Lys Phe
50 55 60
Lys Asp Arg Thr Ile Leu Thr Ala Asp Arg Ser Ser Thr Thr Ala Tyr
65 70 75 80
Met Gln Leu Ser Ser Leu Thr Ser Glu Asp Ser Ala Val Tyr Tyr Cys
85 90 95
Ser Arg Lys Gly Glu Lys Leu Leu Gly Asn Arg Tyr Trp Tyr Phe Asp
100 105 110
Val Trp Gly Ala Gly Thr Ser Val Thr Val Ser Ser Ala Ser Val Ala
115 120 125
Ala Pro Ser Val Phe Ile Phe Pro Pro Ser Asp Glu Gln Leu Lys Ser
130 135 140
Gly Thr Ala Ser Val Val Cys Leu Leu Asn Asn Phe Tyr Pro Arg Glu
145 150 155 160
Ala Lys Val Gln Trp Lys Val Asp Asn Ala Leu Gln Ser Gly Asn Ser
165 170 175
Gln Glu Ser Val Thr Glu Gln Asp Ser Lys Asp Ser Thr Tyr Ser Leu
180 185 190
Ser Ser Thr Leu Thr Leu Ser Lys Ala Asp Tyr Glu Lys His Lys Val
195 200 205
Tyr Ala Cys Glu Val Thr His Gln Gly Leu Ser Ser Pro Val Thr Lys
210 215 220
Ser Phe Asn Arg Gly Glu Cys
225 230

SEQ ID NO: 38
<212> DNA
<223> FN18 (VH-CL)
caggtgcagc tgcagcagag cgaggccgag ctggctagac ctggagccag cgtgaagatg 60
agctgcaagg ccagcggcta caccttcacc gactacacca tccactggct gaagcagcgg 120
cctggacagg gcctggactg gatcggctac ttcaacccca gcagcgagag caccgagtac 180
aaccggaagt tcaaggaccg gaccatcctg accgccgaca gaagcagcac caccgcctac 240
atgcagctga gcagcctgac cagcgaggac agcgccgtgt actactgcag ccggaagggc 300
gagaagctgc tgggcaacag atactggtac ttcgacgtgt ggggagccgg caccagcgtg 360
accgtgtcta gcgctagcgt ggctgcacca tctgtcttca tcttcccgcc atctgatgag 420
cagttgaaat ctggaactgc ctctgttgtg tgcctgctga ataacttcta tcccagagag 480
gccaaagtac agtggaaggt ggataacgcc ctccaatcgg gtaactccca ggagagtgtc 540
acagagcagg acagcaagga cagcacctac agcctcagca gcaccctgac gctgagcaaa 600
gcagactacg agaaacacaa agtctacgcc tgcgaagtca cccatcaggg cctgagctcg 660
cccgtcacaa agagcttcaa caggggagag tgttga 696

SEQ ID NO: 39
<212> PRT
<223> 2C11 (VL-CH1)-LC007 (VH-CH1)-Fc(hole) P329G LALA
Asp Ile Gln Met Thr Gln Ser Pro Ser Ser Leu Pro Ala Ser Leu Gly
1 5 10 15
Asp Arg Val Thr Ile Asn Cys Gln Ala Ser Gln Asp Ile Ser Asn Tyr
20 25 30
Leu Asn Trp Tyr Gln Gln Lys Pro Gly Lys Ala Pro Lys Leu Leu Ile
35 40 45
Tyr Tyr Thr Asn Lys Leu Ala Asp Gly Val Pro Ser Arg Phe Ser Gly
50 55 60
Ser Gly Ser Gly Arg Asp Ser Ser Phe Thr Ile Ser Ser Leu Glu Ser
65 70 75 80
Glu Asp Ile Gly Ser Tyr Tyr Cys Gln Gln Tyr Tyr Asn Tyr Pro Trp
85 90 95
Thr Phe Gly Pro Gly Thr Lys Leu Glu Ile Lys Ser Ser Ala Ser Thr
100 105 110
Lys Gly Pro Ser Val Phe Pro Leu Ala Pro Ser Ser Lys Ser Thr Ser
115 120 125
Gly Gly Thr Ala Ala Leu Gly Cys Leu Val Lys Asp Tyr Phe Pro Glu
130 135 140
Pro Val Thr Val Ser Trp Asn Ser Gly Ala Leu Thr Ser Gly Val His
145 150 155 160
Thr Phe Pro Ala Val Leu Gln Ser Ser Gly Leu Tyr Ser Leu Ser Ser
165 170 175
Val Val Thr Val Pro Ser Ser Ser Leu Gly Thr Gly Thr Tyr Ile Cys
180 185 190
Asn Val Asn His Lys Pro Ser Asn Thr Lys Val Asp Lys Lys Val Glu
195 200 205
Pro Lys Ser Cys Gly Gly Gly Gly Ser Gly Gly Gly Gly Ser Glu Val
210 215 220
Gln Leu Gln Glu Ser Gly Pro Gly Leu Val Lys Pro Ser Gln Ser Leu
225 230 235 240
Ser Leu Thr Cys Ser Val Thr Gly Tyr Ser Ile Thr Ser Gly Tyr Tyr
245 250 255
Trp Asn Trp Ile Arg Gln Phe Pro Gly Asn Lys Leu Glu Trp Met Gly
260 265 270
Tyr Ile Thr Tyr Asp Gly Ser Asn Asn Tyr Asn Pro Ser Leu Lys Asn
275 280 285
Arg Ile Ser Ile Thr Arg Asp Thr Ser Lys Asn Gln Phe Phe Leu Lys
290 295 300
Leu Asn Ser Val Thr Thr Glu Asp Thr Ala Thr Tyr Tyr Cys Ala Asp
305 310 315 320
Phe Asp Tyr Trp Gly Gln Gly Thr Thr Leu Thr Val Ser Ser Ala Ser
325 330 335
Thr Lys Gly Pro Ser Val Phe Pro Leu Ala Pro Ser Ser Lys Ser Thr
340 345 350
Ser Gly Gly Thr Ala Ala Leu Gly Cys Leu Val Lys Asp Tyr Phe Pro
355 360 365
Glu Pro Val Thr Val Ser Trp Asn Ser Gly Ala Leu Thr Ser Gly Val
370 375 380
His Thr Phe Pro Ala Val Leu Gln Ser Ser Gly Leu Tyr Ser Leu Ser
385 390 395 400
Ser Val Val Thr Val Pro Ser Ser Ser Leu Gly Thr Gln Thr Tyr Ile
405 410 415
Cys Asn Val Asn His Lys Pro Ser Asn Thr Lys Val Asp Lys Lys Val
420 425 430
Glu Pro Lys Ser Cys Asp Lys Thr His Thr Cys Pro Pro Cys Pro Ala
435 440 445
Pro Glu Ala Ala Gly Gly Pro Ser Val Phe Leu Phe Pro Pro Lys Pro
450 455 460
Lys Asp Thr Leu Met Ile Ser Arg Thr Pro Glu Val Thr Cys Val Val
465 470 475 480
Val Asp Val Ser His Glu Asp Pro Glu Val Lys Phe Asn Trp Tyr Val
485 490 495
Asp Gly Val Glu Val His Asn Ala Lys Thr Lys Pro Arg Glu Glu Gln
500 505 510
Tyr Asn Ser Thr Tyr Arg Val Val Ser Val Leu Thr Val Leu His Gln
515 520 525
Asp Trp Leu Asn Gly Lys Glu Tyr Lys Cys Lys Val Ser Asn Lys Ala
530 535 540
Leu Gly Ala Pro Ile Glu Lys Thr Ile Ser Lys Ala Lys Gly Gln Pro
545 550 555 560
Arg Glu Pro Gln Val Cys Thr Leu Pro Pro Ser Arg Asp Glu Leu Thr
565 570 575
Lys Asn Gln Val Ser Leu Ser Cys Ala Val Lys Gly Phe Tyr Pro Ser
580 585 590
Asp Ile Ala Val Glu Trp Glu Ser Asn Gly Gln Pro Glu Asn Asn Tyr
595 600 605
Lys Thr Thr Pro Pro Val Leu Asp Ser Asp Gly Ser Phe Phe Leu Val
610 615 620
Ser Lys Leu Thr Val Asp Lys Ser Arg Trp Gln Gln Gly Asn Val Phe
625 630 635 640
Ser Cys Ser Val Met His Glu Ala Leu His Asn His Tyr Thr Gln Lys
645 650 655
Ser Leu Ser Leu Ser Pro Gly Lys
660

SEQ ID NO: 40
<212> DNA
<223> 2C11 (VL-CH1)-LC007 (VH-CH1)-Fc(hole) P329G LALA
gacatccaga tgacccagag ccccagcagc ctgcctgcca gcctgggcga cagagtgacc 60
atcaactgcc aggccagcca ggacatcagc aactacctga actggtatca gcagaagcct 120
ggcaaggccc ccaagctgct gatctactac accaacaagc tggccgacgg cgtgcccagc 180
agattcagcg gcagcggctc cggcagagac agcagcttca ccatctccag cctggaaagc 240
gaggacatcg gcagctacta ctgccagcag tactacaact acccctggac cttcggccct 300
ggcaccaagc tggaaatcaa gagcagcgct tccaccaaag gcccttccgt gtttcctctg 360
gctcctagct ccaagtccac ctctggaggc accgctgctc tcggatgcct cgtgaaggat 420
tattttcctg agcctgtgac agtgtcctgg aatagcggag cactgacctc tggagtgcat 480
actttccccg ctgtgctgca gtcctctgga ctgtacagcc tgagcagcgt ggtgacagtg 540
cccagcagca gcctgggcac ccagacctac atctgcaacg tgaaccacaa gcccagcaac 600
accaaggtgg acaagaaggt ggaacccaag tcttgtggcg gaggcggatc cggcggaggg 660
ggatctgagg tgcagctgca ggaaagcggc cctggcctgg tgaaacccag ccagagcctg 720
agcctgacct gcagcgtgac cggctacagc atcaccagcg gctactactg gaactggatc 780
agacagttcc ccggcaacaa gctggaatgg atgggctaca tcacctacga cggcagcaac 840
aactacaacc ccagcctgaa gaacagaatc agcatcaccc gggacaccag caagaaccag 900
ttcttcctga agctgaacag cgtgaccacc gaggacaccg ccacctacta ctgcgccgac 960
ttcgactact ggggccaggg caccaccctg accgtgtcct ccgcctctac caagggcccc 1020
agcgtgttcc ccctggcacc cagcagcaag agcacatctg gcggaacagc cgctctgggc 1080
tgtctggtga aagactactt ccccgagccc gtgaccgtgt cttggaactc tggcgccctg 1140
accagcggcg tgcacacctt tccagccgtg ctgcagagca gcggcctgta ctccctgtcc 1200
tccgtggtca ccgtgccctc tagctccctg ggaacacaga catatatctg taatgtcaat 1260
cacaagcctt ccaacaccaa agtcgataag aaagtcgagc ccaagagctg cgacaaaact 1320
cacacatgcc caccgtgccc agcacctgaa gctgcagggg gaccgtcagt cttcctcttc 1380
cccccaaaac ccaaggacac cctcatgatc tcccggaccc ctgaggtcac atgcgtggtg 1440
gtggacgtga gccacgaaga ccctgaggtc aagttcaact ggtacgtgga cggcgtggag 1500
gtgcataatg ccaagacaaa gccgcgggag gagcagtaca acagcacgta ccgtgtggtc 1560
agcgtcctca ccgtcctgca ccaggactgg ctgaatggca aggagtacaa gtgcaaggtc 1620
tccaacaaag ccctcggcgc ccccatcgag aaaaccatct ccaaagccaa agggcagccc 1680
cgagaaccac aggtgtgcac cctgccccca tcccgggatg agctgaccaa gaaccaggtc 1740
agcctctcgt gcgcagtcaa aggcttctat cccagcgaca tcgccgtgga gtgggagagc 1800
aatgggcagc cggagaacaa ctacaagacc acgcctcccg tgctggactc cgacggctcc 1860
ttcttcctcg tgagcaagct caccgtggac aagagcaggt ggcagcaggg gaacgtcttc 1920
tcatgctccg tgatgcatga ggctctgcac aaccactaca cgcagaagag cctctccctg 1980
tctccgggta aatga 1995

SEQ ID NO: 41
<212> PRT
<223> 2C11 (VH-CL)
Glu Val Gln Leu Val Glu Ser Gly Gly Gly Leu Val Gln Pro Gly Lys
1 5 10 15
Ser Leu Lys Leu Ser Cys Glu Ala Ser Gly Phe Thr Phe Ser Gly Tyr
20 25 30
Gly Met His Trp Val Arg Gln Ala Pro Gly Arg Gly Leu Glu Ser Val
35 40 45
Ala Tyr Ile Thr Ser Ser Ser Ile Asn Ile Lys Tyr Ala Asp Ala Val
50 55 60
Lys Gly Arg Phe Thr Val Ser Arg Asp Asn Ala Lys Asn Leu Leu Phe
65 70 75 80
Leu Gln Met Asn Ile Leu Lys Ser Glu Asp Thr Ala Met Tyr Tyr Cys
85 90 95
Ala Arg Phe Asp Trp Asp Lys Asn Tyr Trp Gly Gln Gly Thr Met Val
100 105 110
Thr Val Ser Ser Ala Ser Val Ala Ala Pro Ser Val Phe Ile Phe Pro
115 120 125
Pro Ser Asp Glu Gln Leu Lys Ser Gly Thr Ala Ser Val Val Cys Leu
130 135 140
Leu Asn Asn Phe Tyr Pro Arg Glu Ala Lys Val Gln Trp Lys Val Asp
145 150 155 160
Asn Ala Leu Gln Ser Gly Asn Ser Gln Glu Ser Val Thr Glu Gln Asp
165 170 175
Ser Lys Asp Ser Thr Tyr Ser Leu Ser Ser Thr Leu Thr Leu Ser Lys
180 185 190
Ala Asp Tyr Glu Lys His Lys Val Tyr Ala Cys Glu Val Thr His Gln
195 200 205
Gly Leu Ser Ser Pro Val Thr Lys Ser Phe Asn Arg Gly Glu Cys
210 215 220

SEQ ID NO: 42
<212> DNA
<223> 2C11 (VH-CL)
gaggtgcagc tggtggaaag cggcggaggc ctggtgcagc ccggcaagag cctgaagctg 60
agctgcgagg ccagcggctt caccttcagc ggctacggca tgcactgggt gagacaggcc 120
cctggcagag gactggaaag cgtggcctac atcaccagca gcagcatcaa cattaagtac 180
gccgacgccg tgaagggccg gttcaccgtg tccagggata acgccaagaa cctgctgttc 240
ctgcagatga acatcctgaa gtccgaggac accgctatgt attactgcgc cagattcgac 300
tgggacaaga actactgggg ccagggcacc atggtcacag tgtctagcgc tagcgtggct 360
gcaccatctg tcttcatctt cccgccatct gatgagcagt tgaaatctgg aactgcctct 420
gttgtgtgcc tgctgaataa cttctatccc agagaggcca aagtacagtg gaaggtggat 480
aacgccctcc aatcgggtaa ctcccaggag agtgtcacag agcaggacag caaggacagc 540
acctacagcc tcagcagcac cctgacgctg agcaaagcag actacgagaa acacaaagtc 600
tacgcctgcg aagtcaccca tcagggcctg agctcgcccg tcacaaagag cttcaacagg 660
ggagagtgtt ga 672

SEQ ID NO: 43
<212> PRT
<223> V9 (scFab)-Fc(knob) P329G LALA
Asp Ile Gln Met Thr Gln Ser Pro Ser Ser Leu Ser Ala Ser Val Gly
1 5 10 15
Asp Arg Val Thr Ile Thr Cys Arg Ala Ser Gln Asp Ile Arg Asn Tyr
20 25 30
Leu Asn Trp Tyr Gln Gln Lys Pro Gly Lys Ala Pro Lys Leu Leu Ile
35 40 45
Tyr Tyr Thr Ser Arg Leu Glu Ser Gly Val Pro Ser Arg Phe Ser Gly
50 55 60
Ser Gly Ser Gly Thr Asp Tyr Thr Leu Thr Ile Ser Ser Leu Gln Pro
65 70 75 80
Glu Asp Phe Ala Thr Tyr Tyr Cys Gln Gln Gly Asn Thr Leu Pro Trp
85 90 95
Thr Phe Gly Gln Gly Thr Lys Val Glu Ile Lys Arg Thr Val Ala Ala
100 105 110
Pro Ser Val Phe Ile Phe Pro Pro Ser Asp Glu Gln Leu Lys Ser Gly
115 120 125
Thr Ala Ser Val Val Cys Leu Leu Asn Asn Phe Tyr Pro Arg Glu Ala
130 135 140
Lys Val Gln Trp Lys Val Asp Asn Ala Leu Gln Ser Gly Asn Ser Gln
145 150 155 160
Glu Ser Val Thr Glu Gln Asp Ser Lys Asp Ser Thr Tyr Ser Leu Ser
165 170 175
Ser Thr Leu Thr Leu Ser Lys Ala Asp Tyr Glu Lys His Lys Val Tyr
180 185 190
Ala Cys Glu Val Thr His Gln Gly Leu Ser Ser Pro Val Thr Lys Ser
195 200 205
Phe Asn Arg Gly Glu Cys Ser Gly Gly Gly Ser Gly Gly Gly Ser Glu
210 215 220
Gly Gly Gly Ser Glu Gly Gly Gly Ser Glu Gly Gly Gly Ser Glu Gly
225 230 235 240
Gly Gly Ser Gly Gly Gly Ser Gly Glu Val Gln Leu Val Glu Ser Gly
245 250 255
Gly Gly Leu Val Gln Pro Gly Gly Ser Leu Arg Leu Ser Cys Ala Ala
260 265 270
Ser Gly Tyr Ser Phe Thr Gly Tyr Thr Met Asn Trp Val Arg Gln Ala
275 280 285
Pro Gly Lys Gly Leu Glu Trp Val Ala Leu Ile Asn Pro Tyr Lys Gly
290 295 300
Val Ser Thr Tyr Asn Gln Lys Phe Lys Asp Arg Phe Thr Ile Ser Val
305 310 315 320
Asp Lys Ser Lys Asn Thr Ala Tyr Leu Gln Met Asn Ser Leu Arg Ala
325 330 335
Glu Asp Thr Ala Val Tyr Tyr Cys Ala Arg Ser Gly Tyr Tyr Gly Asp
340 345 350
Ser Asp Trp Tyr Phe Asp Val Trp Gly Gln Gly Thr Leu Val Thr Val
355 360 365
Ser Ser Ala Ser Thr Lys Gly Pro Ser Val Phe Pro Leu Ala Pro Ser
370 375 380
Ser Lys Ser Thr Ser Gly Gly Thr Ala Ala Leu Gly Cys Leu Val Lys
385 390 395 400
Asp Tyr Phe Pro Glu Pro Val Thr Val Ser Trp Asn Ser Gly Ala Leu
405 410 415
Thr Ser Gly Val His Thr Phe Pro Ala Val Leu Gln Ser Ser Gly Leu
420 425 430
Tyr Ser Leu Ser Ser Val Val Thr Val Pro Ser Ser Ser Leu Gly Thr
435 440 445
Gln Thr Tyr Ile Cys Asn Val Asn His Lys Pro Ser Asn Thr Lys Val
450 455 460
Asp Lys Lys Val Glu Pro Lys Ser Cys Asp Lys Thr His Thr Cys Pro
465 470 475 480
Pro Cys Pro Ala Pro Glu Ala Ala Gly Gly Pro Ser Val Phe Leu Phe
485 490 495
Pro Pro Lys Pro Lys Asp Thr Leu Met Ile Ser Arg Thr Pro Glu Val
500 505 510
Thr Cys Val Val Val Asp Val Ser His Glu Asp Pro Glu Val Lys Phe
515 520 525
Asn Trp Tyr Val Asp Gly Val Glu Val His Asn Ala Lys Thr Lys Pro
530 535 540
Arg Glu Glu Gln Tyr Asn Ser Thr Tyr Arg Val Val Ser Val Leu Thr
545 550 555 560
Val Leu His Gln Asp Trp Leu Asn Gly Lys Glu Tyr Lys Cys Lys Val
565 570 575
Ser Asn Lys Ala Leu Gly Ala Pro Ile Glu Lys Thr Ile Ser Lys Ala
580 585 590
Lys Gly Gln Pro Arg Glu Pro Gln Val Tyr Thr Leu Pro Pro Cys Arg
595 600 605
Asp Glu Leu Thr Lys Asn Gln Val Ser Leu Trp Cys Leu Val Lys Gly
610 615 620
Phe Tyr Pro Ser Asp Ile Ala Val Glu Trp Glu Ser Asn Gly Gln Pro
625 630 635 640
Glu Asn Asn Tyr Lys Thr Thr Pro Pro Val Leu Asp Ser Asp Gly Ser
645 650 655
Phe Phe Leu Tyr Ser Lys Leu Thr Val Asp Lys Ser Arg Trp Gln Gln
660 665 670
Gly Asn Val Phe Ser Cys Ser Val Met His Glu Ala Leu His Asn His
675 680 685
Tyr Thr Gln Lys Ser Leu Ser Leu Ser Pro Gly Lys
690 695 700

SEQ ID NO: 44
<212> DNA
<223> V9 (scFab)-Fc(knob) P329G LALA
gacatccaga tgacccagag cccctctagc ctgagcgcca gcgtgggcga cagagtgacc 60
atcacctgtc gggccagcca ggacatcaga aactacctga actggtatca gcagaagccc 120
ggcaaggccc ccaagctgct gatctactac acctctagac tggaaagcgg cgtgcccagc 180
cggtttagcg gcagcggctc cggcaccgac tacaccctga ccatcagcag cctgcagccc 240
gaggacttcg ccacctacta ctgccagcag ggcaacacac tcccctggac cttcggccag 300
ggcaccaagg tggagatcaa gcgtacggtg gccgctccca gcgtgttcat cttccccccc 360
agcgacgagc agctgaagtc cggcaccgcc agcgtcgtgt gcctgctgaa caacttctac 420
ccccgggagg ccaaggtgca gtggaaggtg gacaacgccc tgcagagcgg caacagccag 480
gaaagcgtca ccgagcagga cagcaaggac tccacctaca gcctgagcag caccctgacc 540
ctgagcaagg ccgactacga gaagcacaag gtgtacgcct gcgaagtgac ccaccagggc 600
ctgtccagcc ccgtgaccaa gagcttcaac cggggcgagt gcagcggcgg aggctctgga 660
ggcggctctg aaggcggagg aagtgagggc ggaggctcag aaggcggcgg aagcgaaggt 720
ggcggctctg gcggcggatc cggcgaggtg cagctggtcg agtccggcgg aggcctggtg 780
cagcctggcg gcagcctgag actgagctgc gccgccagcg gctacagctt caccggctac 840
accatgaact gggtccggca ggctcctggc aagggcctcg aatgggtggc cctgatcaac 900
ccctacaagg gcgtgagcac ctacaaccag aagttcaagg accggttcac catcagcgtg 960
gacaagagca agaacaccgc ctatctgcag atgaacagcc tgcgggccga ggacaccgcc 1020
gtgtactact gcgccagaag cggctactac ggcgacagcg actggtactt cgacgtgtgg 1080
ggccagggca cactggtcac cgtgtccagc gctagcacca agggccctag cgtgttccct 1140
ctggccccta gcagcaagag cacaagtgga ggaacagccg ccctgggctg cctggtcaag 1200
gactacttcc ccgagcccgt gaccgtgtcc tggaattctg gcgccctgac aagcggcgtg 1260
cacacatttc cagccgtgct gcagagcagc ggcctgtact ctctgagcag cgtcgtgacc 1320
gtgccctcta gctctctggg cacccagacc tacatctgca acgtgaacca caagcccagc 1380
aacaccaaag tggacaagaa ggtggaaccc aagagctgcg acaagaccca cacctgtccc 1440
ccttgccctg cccctgaagc tgctggtggc ccttccgtgt tcctgttccc cccaaagccc 1500
aaggacaccc tgatgatcag ccggaccccc gaagtgacct gcgtggtggt cgatgtgtcc 1560
cacgaggacc ctgaagtgaa gttcaattgg tacgtggacg gcgtggaagt gcacaatgcc 1620
aagaccaagc cgcgggagga gcagtacaac agcacgtacc gtgtggtcag cgtcctcacc 1680
gtcctgcacc aggactggct gaatggcaag gagtacaagt gcaaggtctc caacaaagcc 1740
ctcggcgccc ccatcgagaa aaccatctcc aaagccaaag ggcagccccg agaaccacag 1800
gtgtacaccc tgcccccatg ccgggatgag ctgaccaaga accaggtcag cctgtggtgc 1860
ctggtcaaag gcttctatcc cagcgacatc gccgtggagt gggagagcaa tgggcagccg 1920
gagaacaact acaagaccac gcctcccgtg ctggactccg acggctcctt cttcctctac 1980
agcaagctca ccgtggacaa gagcaggtgg cagcagggga acgtcttctc atgctccgtg 2040
atgcatgagg ctctgcacaa ccactacacg cagaagagcc tctccctgtc tccgggtaaa 2100
taa 2103

SEQ ID NO: 45
<212> PRT
<223> GA201 (VH-CH1)-Fc(hole) P329G LALA
Gln Val Gln Leu Val Gln Ser Gly Ala Glu Val Lys Lys Pro Gly Ser
1 5 10 15
Ser Val Lys Val Ser Cys Lys Ala Ser Gly Phe Thr Phe Thr Asp Tyr
20 25 30
Lys Ile His Trp Val Arg Gln Ala Pro Gly Gln Gly Leu Glu Trp Met
35 40 45
Gly Tyr Phe Asn Pro Asn Ser Gly Tyr Ser Thr Tyr Ala Gln Lys Phe
50 55 60
Gln Gly Arg Val Thr Ile Thr Ala Asp Lys Ser Thr Ser Thr Ala Tyr
65 70 75 80
Met Glu Leu Ser Ser Leu Arg Ser Glu Asp Thr Ala Val Tyr Tyr Cys
85 90 95
Ala Arg Leu Ser Pro Gly Gly Tyr Tyr Val Met Asp Ala Trp Gly Gln
100 105 110
Gly Thr Thr Val Thr Val Ser Ser Ala Ser Thr Lys Gly Pro Ser Val
115 120 125
Phe Pro Leu Ala Pro Ser Ser Lys Ser Thr Ser Gly Gly Thr Ala Ala
130 135 140
Leu Gly Cys Leu Val Lys Asp Tyr Phe Pro Glu Pro Val Thr Val Ser
145 150 155 160
Trp Asn Ser Gly Ala Leu Thr Ser Gly Val His Thr Phe Pro Ala Val
165 170 175
Leu Gln Ser Ser Gly Leu Tyr Ser Leu Ser Ser Val Val Thr Val Pro
180 185 190
Ser Ser Ser Leu Gly Thr Gln Thr Tyr Ile Cys Asn Val Asn His Lys
195 200 205
Pro Ser Asn Thr Lys Val Asp Lys Lys Val Glu Pro Lys Ser Cys Asp
210 215 220
Lys Thr His Thr Cys Pro Pro Cys Pro Ala Pro Glu Ala Ala Gly Gly
225 230 235 240
Pro Ser Val Phe Leu Phe Pro Pro Lys Pro Lys Asp Thr Leu Met Ile
245 250 255
Ser Arg Thr Pro Glu Val Thr Cys Val Val Val Asp Val Ser His Glu
260 265 270
Asp Pro Glu Val Lys Phe Asn Trp Tyr Val Asp Gly Val Glu Val His
275 280 285
Asn Ala Lys Thr Lys Pro Arg Glu Glu Gln Tyr Asn Ser Thr Tyr Arg
290 295 300
Val Val Ser Val Leu Thr Val Leu His Gln Asp Trp Leu Asn Gly Lys
305 310 315 320
Glu Tyr Lys Cys Lys Val Ser Asn Lys Ala Leu Gly Ala Pro Ile Glu
325 330 335
Lys Thr Ile Ser Lys Ala Lys Gly Gln Pro Arg Glu Pro Gln Val Cys
340 345 350
Thr Leu Pro Pro Ser Arg Asp Glu Leu Thr Lys Asn Gln Val Ser Leu
355 360 365
Ser Cys Ala Val Lys Gly Phe Tyr Pro Ser Asp Ile Ala Val Glu Trp
370 375 380
Glu Ser Asn Gly Gln Pro Glu Asn Asn Tyr Lys Thr Thr Pro Pro Val
385 390 395 400
Leu Asp Ser Asp Gly Ser Phe Phe Leu Val Ser Lys Leu Thr Val Asp
405 410 415
Lys Ser Arg Trp Gln Gln Gly Asn Val Phe Ser Cys Ser Val Met His
420 425 430
Glu Ala Leu His Asn His Tyr Thr Gln Lys Ser Leu Ser Leu Ser Pro
435 440 445
Gly Lys
450

SEQ ID NO: 46
<212> DNA
<223> GA201 (VH-CH1)-Fc(hole) P329G LALA
caggtgcagc tggtgcagtc tggggctgag gtgaagaagc ctgggtcctc ggtgaaggtc 60
tcctgcaagg cctctggttt cacattcact gactacaaga tacactgggt gcgacaggcc 120
cctggacaag ggctcgagtg gatgggatat ttcaacccta acagcggtta tagtacctac 180
gcacagaagt tccagggcag ggtcaccatt accgcggaca aatccacgag cacagcctac 240
atggagctga gcagcctgag atctgaggac acggccgtgt attactgtgc gagactatcc 300
ccaggcggtt actatgttat ggatgcctgg ggccaaggga ccaccgtgac cgtctcctca 360
gctagcacca agggcccatc ggtcttcccc ctggcaccct cctccaagag cacctctggg 420
ggcacagcgg ccctgggctg cctggtcaag gactacttcc ccgaaccggt gacggtgtcg 480
tggaactcag gcgccctgac cagcggcgtg cacaccttcc cggctgtcct acagtcctca 540
ggactctact ccctcagcag cgtggtgacc gtgccctcca gcagcttggg cacccagacc 600
tacatctgca acgtgaatca caagcccagc aacaccaagg tggacaagaa agttgagccc 660
aaatcttgtg acaaaactca cacatgccca ccgtgcccag cacctgaagc tgcaggggga 720
ccgtcagtct tcctcttccc cccaaaaccc aaggacaccc tcatgatctc ccggacccct 780
gaggtcacat gcgtggtggt ggacgtgagc cacgaagacc ctgaggtcaa gttcaactgg 840
tacgtggacg gcgtggaggt gcataatgcc aagacaaagc cgcgggagga gcagtacaac 900
agcacgtacc gtgtggtcag cgtcctcacc gtcctgcacc aggactggct gaatggcaag 960
gagtacaagt gcaaggtctc caacaaagcc ctcggcgccc ccatcgagaa aaccatctcc 1020
aaagccaaag ggcagccccg agaaccacag gtgtgcaccc tgcccccatc ccgggatgag 1080
ctgaccaaga accaggtcag cctctcgtgc gcagtcaaag gcttctatcc cagcgacatc 1140
gccgtggagt gggagagcaa tgggcagccg gagaacaact acaagaccac gcctcccgtg 1200
ctggactccg acggctcctt cttcctcgtg agcaagctca ccgtggacaa gagcaggtgg 1260
cagcagggga acgtcttctc atgctccgtg atgcatgagg ctctgcacaa ccactacacg 1320
cagaagagcc tctccctgtc tccgggtaaa tga 1353

SEQ ID NO: 47
<212> PRT
<223> GA201 (VL-CL)
Asp Ile Gln Met Thr Gln Ser Pro Ser Ser Leu Ser Ala Ser Val Gly
1 5 10 15
Asp Arg Val Thr Ile Thr Cys Arg Ala Ser Gln Gly Ile Asn Asn Tyr
20 25 30
Leu Asn Trp Tyr Gln Gln Lys Pro Gly Lys Ala Pro Lys Arg Leu Ile
35 40 45
Tyr Asn Thr Asn Asn Leu Gln Thr Gly Val Pro Ser Arg Phe Ser Gly
50 55 60
Ser Gly Ser Gly Thr Glu Phe Thr Leu Thr Ile Ser Ser Leu Gln Pro
65 70 75 80
Glu Asp Phe Ala Thr Tyr Tyr Cys Leu Gln His Asn Ser Phe Pro Thr
85 90 95
Phe Gly Gln Gly Thr Lys Leu Glu Ile Lys Arg Thr Val Ala Ala Pro
100 105 110
Ser Val Phe Ile Phe Pro Pro Ser Asp Glu Gln Leu Lys Ser Gly Thr
115 120 125
Ala Ser Val Val Cys Leu Leu Asn Asn Phe Tyr Pro Arg Glu Ala Lys
130 135 140
Val Gln Trp Lys Val Asp Asn Ala Leu Gln Ser Gly Asn Ser Gln Glu
145 150 155 160
Ser Val Thr Glu Gln Asp Ser Lys Asp Ser Thr Tyr Ser Leu Ser Ser
165 170 175
Thr Leu Thr Leu Ser Lys Ala Asp Tyr Glu Lys His Lys Val Tyr Ala
180 185 190
Cys Glu Val Thr His Gln Gly Leu Ser Ser Pro Val Thr Lys Ser Phe
195 200 205
Asn Arg Gly Glu Cys
210

SEQ ID NO: 48
<212> DNA
<223> GA201 (VL-CL)
gatatccaga tgacccagtc tccatcctcc ctgtctgcat ctgtcggaga ccgggtcacc 60
atcacctgcc gggcaagtca gggcattaac aattacttaa attggtacca gcagaagcca 120
gggaaagccc ctaagcgcct gatctataat accaacaact tgcagacagg cgtcccatca 180
aggttcagcg gcagtggatc cgggacagaa ttcactctca ccatcagcag cctgcagcct 240
gaagattttg ccacctatta ctgcttgcag cataatagtt ttcccacgtt tggccagggc 300
accaagctcg agatcaagcg tacggtggct gcaccatctg tcttcatctt cccgccatct 360
gatgagcagt tgaaatctgg aactgcctct gttgtgtgcc tgctgaataa cttctatccc 420
agagaggcca aagtacagtg gaaggtggat aacgccctcc aatcgggtaa ctcccaggag 480
agtgtcacag agcaggacag caaggacagc acctacagcc tcagcagcac cctgacgctg 540
agcaaagcag actacgagaa acacaaagtc tacgcctgcg aagtcaccca tcagggcctg 600
agctcgcccg tcacaaagag cttcaacagg ggagagtgtt ag 642

SEQ ID NO: 49
<212> PRT
<223> GA201 (scFab)-Fc(knob) P329G LALA
Asp Ile Gln Met Thr Gln Ser Pro Ser Ser Leu Ser Ala Ser Val Gly
1 5 10 15
Asp Arg Val Thr Ile Thr Cys Arg Ala Ser Gln Gly Ile Asn Asn Tyr
20 25 30
Leu Asn Trp Tyr Gln Gln Lys Pro Gly Lys Ala Pro Lys Arg Leu Ile
35 40 45
Tyr Asn Thr Asn Asn Leu Gln Thr Gly Val Pro Ser Arg Phe Ser Gly
50 55 60
Ser Gly Ser Gly Thr Glu Phe Thr Leu Thr Ile Ser Ser Leu Gln Pro
65 70 75 80
Glu Asp Phe Ala Thr Tyr Tyr Cys Leu Gln His Asn Ser Phe Pro Thr
85 90 95
Phe Gly Gln Gly Thr Lys Leu Glu Ile Lys Arg Thr Val Ala Ala Pro
100 105 110
Ser Val Phe Ile Phe Pro Pro Ser Asp Glu Gln Leu Lys Ser Gly Thr
115 120 125
Ala Ser Val Val Cys Leu Leu Asn Asn Phe Tyr Pro Arg Glu Ala Lys
130 135 140
Val Gln Trp Lys Val Asp Asn Ala Leu Gln Ser Gly Asn Ser Gln Glu
145 150 155 160
Ser Val Thr Glu Gln Asp Ser Lys Asp Ser Thr Tyr Ser Leu Ser Ser
165 170 175
Thr Leu Thr Leu Ser Lys Ala Asp Tyr Glu Lys His Lys Val Tyr Ala
180 185 190
Cys Glu Val Thr His Gln Gly Leu Ser Ser Pro Val Thr Lys Ser Phe
195 200 205
Asn Arg Gly Glu Cys Ser Gly Gly Gly Ser Gly Gly Gly Ser Glu Gly
210 215 220
Gly Gly Ser Glu Gly Gly Gly Ser Glu Gly Gly Gly Ser Glu Gly Gly
225 230 235 240
Gly Ser Gly Gly Gly Ser Gly Gln Val Gln Leu Val Gln Ser Gly Ala
245 250 255
Glu Val Lys Lys Pro Gly Ser Ser Val Lys Val Ser Cys Lys Ala Ser
260 265 270
Gly Phe Thr Phe Thr Asp Tyr Lys Ile His Trp Val Arg Gln Ala Pro
275 280 285
Gly Gln Gly Leu Glu Trp Met Gly Tyr Phe Asn Pro Asn Ser Gly Tyr
290 295 300
Ser Thr Tyr Ala Gln Lys Phe Gln Gly Arg Val Thr Ile Thr Ala Asp
305 310 315 320
Lys Ser Thr Ser Thr Ala Tyr Met Glu Leu Ser Ser Leu Arg Ser Glu
325 330 335
Asp Thr Ala Val Tyr Tyr Cys Ala Arg Leu Ser Pro Gly Gly Tyr Tyr
340 345 350
Val Met Asp Ala Trp Gly Gln Gly Thr Thr Val Thr Val Ser Ser Ala
355 360 365
Ser Thr Lys Gly Pro Ser Val Phe Pro Leu Ala Pro Ser Ser Lys Ser
370 375 380
Thr Ser Gly Gly Thr Ala Ala Leu Gly Cys Leu Val Lys Asp Tyr Phe
385 390 395 400
Pro Glu Pro Val Thr Val Ser Trp Asn Ser Gly Ala Leu Thr Ser Gly
405 410 415
Val His Thr Phe Pro Ala Val Leu Gln Ser Ser Gly Leu Tyr Ser Leu
420 425 430
Ser Ser Val Val Thr Val Pro Ser Ser Ser Leu Gly Thr Gln Thr Tyr
435 440 445
Ile Cys Asn Val Asn His Lys Pro Ser Asn Thr Lys Val Asp Lys Lys
450 455 460
Val Glu Pro Lys Ser Cys Asp Lys Thr His Thr Cys Pro Pro Cys Pro
465 470 475 480
Ala Pro Glu Ala Ala Gly Gly Pro Ser Val Phe Leu Phe Pro Pro Lys
485 490 495
Pro Lys Asp Thr Leu Met Ile Ser Arg Thr Pro Glu Val Thr Cys Val
500 505 510
Val Val Asp Val Ser His Glu Asp Pro Glu Val Lys Phe Asn Trp Tyr
515 520 525
Val Asp Gly Val Glu Val His Asn Ala Lys Thr Lys Pro Arg Glu Glu
530 535 540
Gln Tyr Asn Ser Thr Tyr Arg Val Val Ser Val Leu Thr Val Leu His
545 550 555 560
Gln Asp Trp Leu Asn Gly Lys Glu Tyr Lys Cys Lys Val Ser Asn Lys
565 570 575
Ala Leu Gly Ala Pro Ile Glu Lys Thr Ile Ser Lys Ala Lys Gly Gln
580 585 590
Pro Arg Glu Pro Gln Val Tyr Thr Leu Pro Pro Cys Arg Asp Glu Leu
595 600 605
Thr Lys Asn Gln Val Ser Leu Trp Cys Leu Val Lys Gly Phe Tyr Pro
610 615 620
Ser Asp Ile Ala Val Glu Trp Glu Ser Asn Gly Gln Pro Glu Asn Asn
625 630 635 640
Tyr Lys Thr Thr Pro Pro Val Leu Asp Ser Asp Gly Ser Phe Phe Leu
645 650 655
Tyr Ser Lys Leu Thr Val Asp Lys Ser Arg Trp Gln Gln Gly Asn Val
660 665 670
Phe Ser Cys Ser Val Met His Glu Ala Leu His Asn His Tyr Thr Gln
675 680 685
Lys Ser Leu Ser Leu Ser Pro Gly Lys
690 695

SEQ ID NO: 50
<212> DNA
<223> GA201 (scFab)-Fc(knob) P329G LALA
gatatccaga tgacccagtc tccatcctcc ctgtctgcat ctgtcggaga ccgggtcacc 60
atcacctgcc gggcaagtca gggcattaac aattacttaa attggtacca gcagaagcca 120
gggaaagccc ctaagcgcct gatctataat accaacaact tgcagacagg cgtcccatca 180
aggttcagcg gcagtggatc cgggacagaa ttcactctca ccatcagcag cctgcagcct 240
gaagattttg ccacctatta ctgcttgcag cataatagtt ttcccacgtt tggccagggc 300
accaagctcg agatcaagcg tacggtggcc gctcccagcg tgttcatctt cccccccagc 360
gacgagcagc tgaaatctgg caccgccagc gtcgtgtgcc tgctgaacaa cttctacccc 420
cgggaggcca aggtgcagtg gaaggtggac aacgccctgc agagcggcaa cagccaggaa 480
agcgtcaccg agcaggacag caaggactcc acctatagcc tgtccagcac cctgaccctg 540
agcaaggccg actacgagaa gcacaaggtg tacgcctgcg aagtgaccca ccagggcctg 600
agcagccccg tgaccaagag cttcaaccgg ggcgagtgca gcggcggagg tagcggaggc 660
ggctctgagg gcggaggaag cgagggcgga ggctccgaag gcggcggaag cgaaggtggc 720
ggctctggcg gcggatccgg ccaggtgcag ctggtgcagt ctggggctga ggtgaagaag 780
cctgggtcct cggtgaaggt ctcctgcaag gcctctggtt tcacattcac tgactacaag 840
atacactggg tgcgacaggc ccctggacaa gggctcgagt ggatgggata tttcaaccct 900
aacagcggtt atagtaccta cgcacagaag ttccagggca gggtcaccat taccgcggac 960
aaatccacga gcacagccta catggagctg agcagcctga gatctgagga cacggccgtg 1020
tattactgtg cgagactatc cccaggcggt tactatgtta tggatgcctg gggccaaggg 1080
accaccgtga ccgtctcctc agctagcacc aagggcccta gcgtgttccc tctggcccct 1140
agcagcaaga gcacaagtgg aggaacagcc gccctgggct gcctggtcaa ggactacttc 1200
cccgagcccg tgaccgtgtc ctggaattct ggcgccctga caagcggcgt gcacacattt 1260
ccagccgtgc tgcagagcag cggcctgtac tctctgagca gcgtcgtgac cgtgccctct 1320
agctctctgg gcacccagac ctacatctgc aacgtgaacc acaagcccag caacaccaaa 1380
gtggacaaga aggtggaacc caagagctgc gacaagaccc acacctgtcc cccttgccct 1440
gcccctgaag ctgctggtgg cccttccgtg ttcctgttcc ccccaaagcc caaggacacc 1500
ctgatgatca gccggacccc cgaagtgacc tgcgtggtgg tcgatgtgtc ccacgaggac 1560
cctgaagtga agttcaattg gtacgtggac ggcgtggaag tgcacaatgc caagaccaag 1620
ccgcgggagg agcagtacaa cagcacgtac cgtgtggtca gcgtcctcac cgtcctgcac 1680
caggactggc tgaatggcaa ggagtacaag tgcaaggtct ccaacaaagc cctcggcgcc 1740
cccatcgaga aaaccatctc caaagccaaa gggcagcccc gagaaccaca ggtgtacacc 1800
ctgcccccat gccgggatga gctgaccaag aaccaggtca gcctgtggtg cctggtcaaa 1860
ggcttctatc ccagcgacat cgccgtggag tgggagagca atgggcagcc ggagaacaac 1920
tacaagacca cgcctcccgt gctggactcc gacggctcct tcttcctcta cagcaagctc 1980
accgtggaca agagcaggtg gcagcagggg aacgtcttct catgctccgt gatgcatgag 2040
gctctgcaca accactacac gcagaagagc ctctccctgt ctccgggtaa ataa 2094

SEQ ID NO: 51
<212> PRT
<223> V9 (VH-CH1)-Fc(hole) P329G LALA
Glu Val Gln Leu Val Glu Ser Gly Gly Gly Leu Val Gln Pro Gly Gly
1 5 10 15
Ser Leu Arg Leu Ser Cys Ala Ala Ser Gly Tyr Ser Phe Thr Gly Tyr
20 25 30
Thr Met Asn Trp Val Arg Gln Ala Pro Gly Lys Gly Leu Glu Trp Val
35 40 45
Ala Leu Ile Asn Pro Tyr Lys Gly Val Ser Thr Tyr Asn Gln Lys Phe
50 55 60
Lys Asp Arg Phe Thr Ile Ser Val Asp Lys Ser Lys Asn Thr Ala Tyr
65 70 75 80
Leu Gln Met Asn Ser Leu Arg Ala Glu Asp Thr Ala Val Tyr Tyr Cys
85 90 95
Ala Arg Ser Gly Tyr Tyr Gly Asp Ser Asp Trp Tyr Phe Asp Val Trp
100 105 110
Gly Gln Gly Thr Leu Val Thr Val Ser Ser Ala Ser Thr Lys Gly Pro
115 120 125
Ser Val Phe Pro Leu Ala Pro Ser Ser Lys Ser Thr Ser Gly Gly Thr
130 135 140
Ala Ala Leu Gly Cys Leu Val Lys Asp Tyr Phe Pro Glu Pro Val Thr
145 150 155 160
Val Ser Trp Asn Ser Gly Ala Leu Thr Ser Gly Val His Thr Phe Pro
165 170 175
Ala Val Leu Gln Ser Ser Gly Leu Tyr Ser Leu Ser Ser Val Val Thr
180 185 190
Val Pro Ser Ser Ser Leu Gly Thr Gln Thr Tyr Ile Cys Asn Val Asn
195 200 205
His Lys Pro Ser Asn Thr Lys Val Asp Lys Lys Val Glu Pro Lys Ser
210 215 220
Cys Asp Lys Thr His Thr Cys Pro Pro Cys Pro Ala Pro Glu Ala Ala
225 230 235 240
Gly Gly Pro Ser Val Phe Leu Phe Pro Pro Lys Pro Lys Asp Thr Leu
245 250 255
Met Ile Ser Arg Thr Pro Glu Val Thr Cys Val Val Val Asp Val Ser
260 265 270
His Glu Asp Pro Glu Val Lys Phe Asn Trp Tyr Val Asp Gly Val Glu
275 280 285
Val His Asn Ala Lys Thr Lys Pro Arg Glu Glu Gln Tyr Asn Ser Thr
290 295 300
Tyr Arg Val Val Ser Val Leu Thr Val Leu His Gln Asp Trp Leu Asn
305 310 315 320
Gly Lys Glu Tyr Lys Cys Lys Val Ser Asn Lys Ala Leu Gly Ala Pro
325 330 335
Ile Glu Lys Thr Ile Ser Lys Ala Lys Gly Gln Pro Arg Glu Pro Gln
340 345 350
Val Cys Thr Leu Pro Pro Ser Arg Asp Glu Leu Thr Lys Asn Gln Val
355 360 365
Ser Leu Ser Cys Ala Val Lys Gly Phe Tyr Pro Ser Asp Ile Ala Val
370 375 380
Glu Trp Glu Ser Asn Gly Gln Pro Glu Asn Asn Tyr Lys Thr Thr Pro
385 390 395 400
Pro Val Leu Asp Ser Asp Gly Ser Phe Phe Leu Val Ser Lys Leu Thr
405 410 415
Val Asp Lys Ser Arg Trp Gln Gln Gly Asn Val Phe Ser Cys Ser Val
420 425 430
Met His Glu Ala Leu His Asn His Tyr Thr Gln Lys Ser Leu Ser Leu
435 440 445
Ser Pro Gly Lys
450

SEQ ID NO: 52
<212> DNA
<223> V9 (VH-CH1)-Fc(hole) P329G LALA
gaggtgcagc tggtcgagag cggaggcggc ctggtgcagc ctggcggcag cctgagactg 60
agctgcgccg ccagcggcta cagcttcacc ggctacacca tgaactgggt ccggcaggca 120
cctggcaagg gactggaatg ggtggccctg atcaacccct acaagggcgt gagcacctac 180
aaccagaagt tcaaggaccg gttcaccatc agcgtggaca agagcaagaa caccgcctat 240
ctgcagatga acagcctgcg ggccgaggac accgccgtgt actactgcgc cagaagcggc 300
tactacggcg acagcgactg gtacttcgac gtgtggggcc agggcaccct cgtgaccgtg 360
tctagcgcta gcaccaaggg cccctccgtg ttccccctgg cccccagcag caagagcacc 420
agcggcggca cagccgctct gggctgcctg gtcaaggact acttccccga gcccgtgacc 480
gtgtcctgga acagcggagc cctgacctcc ggcgtgcaca ccttccccgc cgtgctgcag 540
agttctggcc tgtatagcct gagcagcgtg gtcaccgtgc cttctagcag cctgggcacc 600
cagacctaca tctgcaacgt gaaccacaag cccagcaaca ccaaggtgga caagaaggtg 660
gagcccaaga gctgcgacaa aactcacaca tgcccaccgt gcccagcacc tgaagctgca 720
gggggaccgt cagtcttcct cttcccccca aaacccaagg acaccctcat gatctcccgg 780
acccctgagg tcacatgcgt ggtggtggac gtgagccacg aagaccctga ggtcaagttc 840
aactggtacg tggacggcgt ggaggtgcat aatgccaaga caaagccgcg ggaggagcag 900
tacaacagca cgtaccgtgt ggtcagcgtc ctcaccgtcc tgcaccagga ctggctgaat 960
ggcaaggagt acaagtgcaa ggtctccaac aaagccctcg gcgcccccat cgagaaaacc 1020
atctccaaag ccaaagggca gccccgagaa ccacaggtgt gcaccctgcc cccatcccgg 1080
gatgagctga ccaagaacca ggtcagcctc tcgtgcgcag tcaaaggctt ctatcccagc 1140
gacatcgccg tggagtggga gagcaatggg cagccggaga acaactacaa gaccacgcct 1200
cccgtgctgg actccgacgg ctccttcttc ctcgtgagca agctcaccgt ggacaagagc 1260
aggtggcagc aggggaacgt cttctcatgc tccgtgatgc atgaggctct gcacaaccac 1320
tacacgcaga agagcctctc cctgtctccg ggtaaatga 1359

SEQ ID NO: 53
<212> PRT
<223> V9 (scFab)-GA201 (VH-CH1)-Fc(knob) P329G LALA
Asp Ile Gln Met Thr Gln Ser Pro Ser Ser Leu Ser Ala Ser Val Gly
1 5 10 15
Asp Arg Val Thr Ile Thr Cys Arg Ala Ser Gln Asp Ile Arg Asn Tyr
20 25 30
Leu Asn Trp Tyr Gln Gln Lys Pro Gly Lys Ala Pro Lys Leu Leu Ile
35 40 45
Tyr Tyr Thr Ser Arg Leu Glu Ser Gly Val Pro Ser Arg Phe Ser Gly
50 55 60
Ser Gly Ser Gly Thr Asp Tyr Thr Leu Thr Ile Ser Ser Leu Gln Pro
65 70 75 80
Glu Asp Phe Ala Thr Tyr Tyr Cys Gln Gln Gly Asn Thr Leu Pro Trp
85 90 95
Thr Phe Gly Gln Gly Thr Lys Val Glu Ile Lys Arg Thr Val Ala Ala
100 105 110
Pro Ser Val Phe Ile Phe Pro Pro Ser Asp Glu Gln Leu Lys Ser Gly
115 120 125
Thr Ala Ser Val Val Cys Leu Leu Asn Asn Phe Tyr Pro Arg Glu Ala
130 135 140
Lys Val Gln Trp Lys Val Asp Asn Ala Leu Gln Ser Gly Asn Ser Gln
145 150 155 160
Glu Ser Val Thr Glu Gln Asp Ser Lys Asp Ser Thr Tyr Ser Leu Ser
165 170 175
Ser Thr Leu Thr Leu Ser Lys Ala Asp Tyr Glu Lys His Lys Val Tyr
180 185 190
Ala Cys Glu Val Thr His Gln Gly Leu Ser Ser Pro Val Thr Lys Ser
195 200 205
Phe Asn Arg Gly Glu Cys Ser Gly Gly Gly Ser Gly Gly Gly Ser Glu
210 215 220
Gly Gly Gly Ser Glu Gly Gly Gly Ser Glu Gly Gly Gly Ser Glu Gly
225 230 235 240
Gly Gly Ser Gly Gly Gly Ser Gly Glu Val Gln Leu Val Glu Ser Gly
245 250 255
Gly Gly Leu Val Gln Pro Gly Gly Ser Leu Arg Leu Ser Cys Ala Ala
260 265 270
Ser Gly Tyr Ser Phe Thr Gly Tyr Thr Met Asn Trp Val Arg Gln Ala
275 280 285
Pro Gly Lys Gly Leu Glu Trp Val Ala Leu Ile Asn Pro Tyr Lys Gly
290 295 300
Val Ser Thr Tyr Asn Gln Lys Phe Lys Asp Arg Phe Thr Ile Ser Val
305 310 315 320
Asp Lys Ser Lys Asn Thr Ala Tyr Leu Gln Met Asn Ser Leu Arg Ala
325 330 335
Glu Asp Thr Ala Val Tyr Tyr Cys Ala Arg Ser Gly Tyr Tyr Gly Asp
340 345 350
Ser Asp Trp Tyr Phe Asp Val Trp Gly Gln Gly Thr Leu Val Thr Val
355 360 365
Ser Ser Ala Ser Thr Lys Gly Pro Ser Val Phe Pro Leu Ala Pro Ser
370 375 380
Ser Lys Ser Thr Ser Gly Gly Thr Ala Ala Leu Gly Cys Leu Val Lys
385 390 395 400
Asp Tyr Phe Pro Glu Pro Val Thr Val Ser Trp Asn Ser Gly Ala Leu
405 410 415
Thr Ser Gly Val His Thr Phe Pro Ala Val Leu Gln Ser Ser Gly Leu
420 425 430
Tyr Ser Leu Ser Ser Val Val Thr Val Pro Ser Ser Ser Leu Gly Thr
435 440 445
Gln Thr Tyr Ile Cys Asn Val Asn His Lys Pro Ser Asn Thr Lys Val
450 455 460
Asp Lys Lys Val Glu Pro Lys Ser Cys Asp Gly Gly Gly Gly Ser Gly
465 470 475 480
Gly Gly Gly Ser Gln Val Gln Leu Val Gln Ser Gly Ala Glu Val Lys
485 490 495
Lys Pro Gly Ser Ser Val Lys Val Ser Cys Lys Ala Ser Gly Phe Thr
500 505 510
Phe Thr Asp Tyr Lys Ile His Trp Val Arg Gln Ala Pro Gly Gln Gly
515 520 525
Leu Glu Trp Met Gly Tyr Phe Asn Pro Asn Ser Gly Tyr Ser Thr Tyr
530 535 540
Ala Gln Lys Phe Gln Gly Arg Val Thr Ile Thr Ala Asp Lys Ser Thr
545 550 555 560
Ser Thr Ala Tyr Met Glu Leu Ser Ser Leu Arg Ser Glu Asp Thr Ala
565 570 575
Val Tyr Tyr Cys Ala Arg Leu Ser Pro Gly Gly Tyr Tyr Val Met Asp
580 585 590
Ala Trp Gly Gln Gly Thr Thr Val Thr Val Ser Ser Ala Ser Thr Lys
595 600 605
Gly Pro Ser Val Phe Pro Leu Ala Pro Ser Ser Lys Ser Thr Ser Gly
610 615 620
Gly Thr Ala Ala Leu Gly Cys Leu Val Lys Asp Tyr Phe Pro Glu Pro
625 630 635 640
Val Thr Val Ser Trp Asn Ser Gly Ala Leu Thr Ser Gly Val His Thr
645 650 655
Phe Pro Ala Val Leu Gln Ser Ser Gly Leu Tyr Ser Leu Ser Ser Val
660 665 670
Val Thr Val Pro Ser Ser Ser Leu Gly Thr Gln Thr Tyr Ile Cys Asn
675 680 685
Val Asn His Lys Pro Ser Asn Thr Lys Val Asp Lys Lys Val Glu Pro
690 695 700
Lys Ser Cys Asp Lys Thr His Thr Cys Pro Pro Cys Pro Ala Pro Glu
705 710 715 720
Ala Ala Gly Gly Pro Ser Val Phe Leu Phe Pro Pro Lys Pro Lys Asp
725 730 735
Thr Leu Met Ile Ser Arg Thr Pro Glu Val Thr Cys Val Val Val Asp
740 745 750
Val Ser His Glu Asp Pro Glu Val Lys Phe Asn Trp Tyr Val Asp Gly
755 760 765
Val Glu Val His Asn Ala Lys Thr Lys Pro Arg Glu Glu Gln Tyr Asn
770 775 780
Ser Thr Tyr Arg Val Val Ser Val Leu Thr Val Leu His Gln Asp Trp
785 790 795 800
Leu Asn Gly Lys Glu Tyr Lys Cys Lys Val Ser Asn Lys Ala Leu Gly
805 810 815
Ala Pro Ile Glu Lys Thr Ile Ser Lys Ala Lys Gly Gln Pro Arg Glu
820 825 830
Pro Gln Val Tyr Thr Leu Pro Pro Cys Arg Asp Glu Leu Thr Lys Asn
835 840 845
Gln Val Ser Leu Trp Cys Leu Val Lys Gly Phe Tyr Pro Ser Asp Ile
850 855 860
Ala Val Glu Trp Glu Ser Asn Gly Gln Pro Glu Asn Asn Tyr Lys Thr
865 870 875 880
Thr Pro Pro Val Leu Asp Ser Asp Gly Ser Phe Phe Leu Tyr Ser Lys
885 890 895
Leu Thr Val Asp Lys Ser Arg Trp Gln Gln Gly Asn Val Phe Ser Cys
900 905 910
Ser Val Met His Glu Ala Leu His Asn His Tyr Thr Gln Lys Ser Leu
915 920 925
Ser Leu Ser Pro Gly Lys
930

SEQ ID NO: 54
<212> DNA
<223> V9 (scFab)-GA201 (VH-CH1)-Fc(knob) P329G LALA
gacatccaga tgacccagag cccctctagc ctgagcgcca gcgtgggcga cagagtgacc 60
atcacctgtc gggccagcca ggacatcaga aactacctga actggtatca gcagaagccc 120
ggcaaggccc ccaagctgct gatctactac acctctagac tggaaagcgg cgtgcccagc 180
cggtttagcg gcagcggctc cggcaccgac tacaccctga ccatcagcag cctgcagccc 240
gaggacttcg ccacctacta ctgccagcag ggcaacacac tcccctggac cttcggccag 300
ggcaccaagg tggagatcaa gcgtacggtg gccgctccca gcgtgttcat cttccccccc 360
agcgacgagc agctgaagtc cggcaccgcc agcgtcgtgt gcctgctgaa caacttctac 420
ccccgggagg ccaaggtgca gtggaaggtg gacaacgccc tgcagagcgg caacagccag 480
gaaagcgtca ccgagcagga cagcaaggac tccacctaca gcctgagcag caccctgacc 540
ctgagcaagg ccgactacga gaagcacaag gtgtacgcct gcgaagtgac ccaccagggc 600
ctgtccagcc ccgtgaccaa gagcttcaac cggggcgagt gcagcggcgg aggctctgga 660
ggcggctctg aaggcggagg aagtgagggc ggaggctcag aaggcggcgg aagcgaaggt 720
ggcggctctg gcggcggatc cggcgaggtg cagctggtcg agtccggcgg aggcctggtg 780
cagcctggcg gcagcctgag actgagctgc gccgccagcg gctacagctt caccggctac 840
accatgaact gggtccggca ggctcctggc aagggcctcg aatgggtggc cctgatcaac 900
ccctacaagg gcgtgagcac ctacaaccag aagttcaagg accggttcac catcagcgtg 960
gacaagagca agaacaccgc ctatctgcag atgaacagcc tgcgggccga ggacaccgcc 1020
gtgtactact gcgccagaag cggctactac ggcgacagcg actggtactt cgacgtgtgg 1080
ggccagggca cactggtcac cgtgtccagc gctagcacca agggcccctc cgtgttcccc 1140
ctggccccca gcagcaagag caccagcggc ggcacagccg ccctcggctg cctggtcaag 1200
gactacttcc ccgagcccgt gaccgtgtcc tggaacagcg gagccctgac ctccggcgtg 1260
cacaccttcc ccgccgtgct gcagagcagc ggcctgtaca gcctgtccag cgtggtcacc 1320
gtgccctcca gcagcctggg cacccagacc tacatctgca acgtgaacca caagcccagc 1380
aataccaagg tggacaagaa ggtggagccc aagagctgcg acggcggtgg tggctccgga 1440
ggcggtggat ctcaggtgca gctggtgcag tctggggctg aggtgaagaa gcctgggtcc 1500
tcggtgaagg tctcctgcaa ggcctctggt ttcacattca ctgactacaa gatacactgg 1560
gtgcgacagg cccctggaca agggctcgag tggatgggat atttcaaccc taacagcggt 1620
tatagtacct acgcacagaa gttccagggc agggtcacca ttaccgcgga caaatccacg 1680
agcacagcct acatggagct gagcagcctg agatctgagg acacggccgt gtattactgt 1740
gcgagactat ccccaggcgg ttactatgtt atggatgcct ggggccaagg gaccaccgtg 1800
accgtctcct cagctagcac caagggcccc tccgtgttcc ccctggcccc cagcagcaag 1860
agcaccagcg gcggcacagc cgctctgggc tgcctggtca aggactactt ccccgagccc 1920
gtgaccgtgt cctggaacag cggagccctg acctccggcg tgcacacctt ccccgccgtg 1980
ctgcagagtt ctggcctgta tagcctgagc agcgtggtca ccgtgccttc tagcagcctg 2040
ggcacccaga cctacatctg caacgtgaac cacaagccca gcaacaccaa ggtggacaag 2100
aaggtggagc ccaagagctg cgacaaaact cacacatgcc caccgtgccc agcacctgaa 2160
gctgcagggg gaccgtcagt cttcctcttc cccccaaaac ccaaggacac cctcatgatc 2220
tcccggaccc ctgaggtcac atgcgtggtg gtggacgtga gccacgaaga ccctgaggtc 2280
aagttcaact ggtacgtgga cggcgtggag gtgcataatg ccaagacaaa gccgcgggag 2340
gagcagtaca acagcacgta ccgtgtggtc agcgtcctca ccgtcctgca ccaggactgg 2400
ctgaatggca aggagtacaa gtgcaaggtc tccaacaaag ccctcggcgc ccccatcgag 2460
aaaaccatct ccaaagccaa agggcagccc cgagaaccac aggtgtacac cctgccccca 2520
tgccgggatg agctgaccaa gaaccaggtc agcctgtggt gcctggtcaa aggcttctat 2580
cccagcgaca tcgccgtgga gtgggagagc aatgggcagc cggagaacaa ctacaagacc 2640
acgcctcccg tgctggactc cgacggctcc ttcttcctct acagcaagct caccgtggac 2700
aagagcaggt ggcagcaggg gaacgtcttc tcatgctccg tgatgcatga ggctctgcac 2760
aaccactaca cgcagaagag cctctccctg tctccgggta aatga 2805

SEQ ID NO: 55
<212> PRT
<223> 3F2 (scFab)-Fc(knob) P329G LALA
Glu Ile Val Leu Thr Gln Ser Pro Gly Thr Leu Ser Leu Ser Pro Gly
1 5 10 15
Glu Arg Ala Thr Leu Ser Cys Arg Ala Ser Gln Ser Val Thr Ser Ser
20 25 30
Tyr Leu Ala Trp Tyr Gln Gln Lys Pro Gly Gln Ala Pro Arg Leu Leu
35 40 45
Ile Asn Val Gly Ser Arg Arg Ala Thr Gly Ile Pro Asp Arg Phe Ser
50 55 60
Gly Ser Gly Ser Gly Thr Asp Phe Thr Leu Thr Ile Ser Arg Leu Glu
65 70 75 80
Pro Glu Asp Phe Ala Val Tyr Tyr Cys Gln Gln Gly Ile Met Leu Pro
85 90 95
Pro Thr Phe Gly Gln Gly Thr Lys Val Glu Ile Lys Arg Thr Val Ala
100 105 110
Ala Pro Ser Val Phe Ile Phe Pro Pro Ser Asp Glu Gln Leu Lys Ser
115 120 125
Gly Thr Ala Ser Val Val Cys Leu Leu Asn Asn Phe Tyr Pro Arg Glu
130 135 140
Ala Lys Val Gln Trp Lys Val Asp Asn Ala Leu Gln Ser Gly Asn Ser
145 150 155 160
Gln Glu Ser Val Thr Glu Gln Asp Ser Lys Asp Ser Thr Tyr Ser Leu
165 170 175
Ser Ser Thr Leu Thr Leu Ser Lys Ala Asp Tyr Glu Lys His Lys Val
180 185 190
Tyr Ala Cys Glu Val Thr His Gln Gly Leu Ser Ser Pro Val Thr Lys
195 200 205
Ser Phe Asn Arg Gly Glu Cys Gly Gly Gly Gly Ser Gly Gly Gly Gly
210 215 220
Ser Gly Gly Gly Gly Ser Gly Gly Gly Gly Ser Gly Gly Gly Gly Ser
225 230 235 240
Gly Gly Gly Gly Ser Gly Gly Glu Val Gln Leu Leu Glu Ser Gly Gly
245 250 255
Gly Leu Val Gln Pro Gly Gly Ser Leu Arg Leu Ser Cys Ala Ala Ser
260 265 270
Gly Phe Thr Phe Ser Ser Tyr Ala Met Ser Trp Val Arg Gln Ala Pro
275 280 285
Gly Lys Gly Leu Glu Trp Val Ser Ala Ile Ser Gly Ser Gly Gly Ser
290 295 300
Thr Tyr Tyr Ala Asp Ser Val Lys Gly Arg Phe Thr Ile Ser Arg Asp
305 310 315 320
Asn Ser Lys Asn Thr Leu Tyr Leu Gln Met Asn Ser Leu Arg Ala Glu
325 330 335
Asp Thr Ala Val Tyr Tyr Cys Ala Lys Gly Trp Phe Gly Gly Phe Asn
340 345 350
Tyr Trp Gly Gln Gly Thr Leu Val Thr Val Ser Ser Ala Ser Thr Lys
355 360 365
Gly Pro Ser Val Phe Pro Leu Ala Pro Ser Ser Lys Ser Thr Ser Gly
370 375 380
Gly Thr Ala Ala Leu Gly Cys Leu Val Lys Asp Tyr Phe Pro Glu Pro
385 390 395 400
Val Thr Val Ser Trp Asn Ser Gly Ala Leu Thr Ser Gly Val His Thr
405 410 415
Phe Pro Ala Val Leu Gln Ser Ser Gly Leu Tyr Ser Leu Ser Ser Val
420 425 430
Val Thr Val Pro Ser Ser Ser Leu Gly Thr Gln Thr Tyr Ile Cys Asn
435 440 445
Val Asn His Lys Pro Ser Asn Thr Lys Val Asp Lys Lys Val Glu Pro
450 455 460
Lys Ser Cys Asp Lys Thr His Thr Cys Pro Pro Cys Pro Ala Pro Glu
465 470 475 480
Ala Ala Gly Gly Pro Ser Val Phe Leu Phe Pro Pro Lys Pro Lys Asp
485 490 495
Thr Leu Met Ile Ser Arg Thr Pro Glu Val Thr Cys Val Val Val Asp
500 505 510
Val Ser His Glu Asp Pro Glu Val Lys Phe Asn Trp Tyr Val Asp Gly
515 520 525
Val Glu Val His Asn Ala Lys Thr Lys Pro Arg Glu Glu Gln Tyr Asn
530 535 540
Ser Thr Tyr Arg Val Val Ser Val Leu Thr Val Leu His Gln Asp Trp
545 550 555 560
Leu Asn Gly Lys Glu Tyr Lys Cys Lys Val Ser Asn Lys Ala Leu Gly
565 570 575
Ala Pro Ile Glu Lys Thr Ile Ser Lys Ala Lys Gly Gln Pro Arg Glu
580 585 590
Pro Gln Val Tyr Thr Leu Pro Pro Cys Arg Asp Glu Leu Thr Lys Asn
595 600 605
Gln Val Ser Leu Trp Cys Leu Val Lys Gly Phe Tyr Pro Ser Asp Ile
610 615 620
Ala Val Glu Trp Glu Ser Asn Gly Gln Pro Glu Asn Asn Tyr Lys Thr
625 630 635 640
Thr Pro Pro Val Leu Asp Ser Asp Gly Ser Phe Phe Leu Tyr Ser Lys
645 650 655
Leu Thr Val Asp Lys Ser Arg Trp Gln Gln Gly Asn Val Phe Ser Cys
660 665 670
Ser Val Met His Glu Ala Leu His Asn His Tyr Thr Gln Lys Ser Leu
675 680 685
Ser Leu Ser Pro Gly Lys
690

SEQ ID NO: 56
<212> DNA
<223> 3F2 (scFab)-Fc(knob) P329G LALA
gagatcgtgc tgacacagag ccccggaacc ctgtctctga gccctggcga aagagccacc 60
ctgagctgta gagccagcca gagcgtgacc agcagctacc tggcctggta tcagcagaag 120
cctggacagg cccccagact gctgatcaat gtgggcagca gacgggccac cggcatccct 180
gatagatttt ctggcagcgg cagcggcacc gacttcaccc tgaccatcag cagactggaa 240
cccgaggact tcgccgtgta ctactgccag cagggcatca tgctgccccc tacatttggc 300
cagggcacca aggtggaaat caagcgtacg gtggccgctc ccagcgtgtt catcttccca 360
cctagcgacg agcagctgaa gtctggcaca gccagcgtcg tgtgcctgct gaacaacttc 420
tacccccgcg aggccaaggt gcagtggaag gtggacaacg ccctgcagag cggcaacagc 480
caggaaagcg tcaccgagca ggacagcaag gactccacct acagcctgag cagcaccctg 540
accctgagca aggccgacta cgagaagcac aaggtgtacg cctgcgaagt gacccaccag 600
ggcctgtcta gccccgtgac caagagcttc aaccggggag aatgtggcgg cggaggatct 660
ggtggcggag gtagtggtgg tggtggatct ggcggaggcg gatccggcgg aggtggaagc 720
ggaggtggtg gaagtggggg agaagtgcag ctgctggaaa gtggcggagg cctggtgcag 780
cctggcggat ctctgagact gagctgtgcc gccagcggct tcacctttag cagctacgcc 840
atgagctggg tccgacaggc ccctggaaag ggactggaat gggtgtccgc catctctggc 900
tctggcggca gcacctacta cgccgatagc gtgaagggcc ggttcaccat cagccgggac 960
aacagcaaga acaccctgta cctgcagatg aacagcctgc gggccgagga taccgccgtg 1020
tattattgcg ccaagggatg gttcggcggc ttcaactatt ggggccaggg aaccctggtc 1080
accgtgtcta gtgctagcac caagggccct agcgtgttcc ctctggcccc tagcagcaag 1140
agcacaagtg gaggaacagc cgccctgggc tgcctggtca aggactactt ccccgagccc 1200
gtgaccgtgt cctggaattc tggcgccctg acaagcggcg tgcacacatt tccagccgtg 1260
ctgcagagca gcggcctgta ctctctgagc agcgtcgtga ccgtgccctc tagctctctg 1320
ggcacccaga cctacatctg caacgtgaac cacaagccca gcaacaccaa agtggacaag 1380
aaggtggaac ccaagagctg cgacaagacc cacacctgtc ccccttgccc tgcccctgaa 1440
gctgctggtg gcccttccgt gttcctgttc cccccaaagc ccaaggacac cctgatgatc 1500
agccggaccc ccgaagtgac ctgcgtggtg gtcgatgtgt cccacgagga ccctgaagtg 1560
aagttcaatt ggtacgtgga cggcgtggaa gtgcacaatg ccaagaccaa gccgcgggag 1620
gagcagtaca acagcacgta ccgtgtggtc agcgtcctca ccgtcctgca ccaggactgg 1680
ctgaatggca aggagtacaa gtgcaaggtc tccaacaaag ccctcggcgc ccccatcgag 1740
aaaaccatct ccaaagccaa agggcagccc cgagaaccac aggtgtacac cctgccccca 1800
tgccgggatg agctgaccaa gaaccaggtc agcctgtggt gcctggtcaa aggcttctat 1860
cccagcgaca tcgccgtgga gtgggagagc aatgggcagc cggagaacaa ctacaagacc 1920
acgcctcccg tgctggactc cgacggctcc ttcttcctct acagcaagct caccgtggac 1980
aagagcaggt ggcagcaggg gaacgtcttc tcatgctccg tgatgcatga ggctctgcac 2040
aaccactaca cgcagaagag cctctccctg tctccgggta aataa 2085

SEQ ID NO: 57
<212> PRT
<223> V9 (scFab)-3F2 (VH-CH1)-Fc(knob) P329G LALA
Asp Ile Gln Met Thr Gln Ser Pro Ser Ser Leu Ser Ala Ser Val Gly
1 5 10 15
Asp Arg Val Thr Ile Thr Cys Arg Ala Ser Gln Asp Ile Arg Asn Tyr
20 25 30
Leu Asn Trp Tyr Gln Gln Lys Pro Gly Lys Ala Pro Lys Leu Leu Ile
35 40 45
Tyr Tyr Thr Ser Arg Leu Glu Ser Gly Val Pro Ser Arg Phe Ser Gly
50 55 60
Ser Gly Ser Gly Thr Asp Tyr Thr Leu Thr Ile Ser Ser Leu Gln Pro
65 70 75 80
Glu Asp Phe Ala Thr Tyr Tyr Cys Gln Gln Gly Asn Thr Leu Pro Trp
85 90 95
Thr Phe Gly Gln Gly Thr Lys Val Glu Ile Lys Arg Thr Val Ala Ala
100 105 110
Pro Ser Val Phe Ile Phe Pro Pro Ser Asp Glu Gln Leu Lys Ser Gly
115 120 125
Thr Ala Ser Val Val Cys Leu Leu Asn Asn Phe Tyr Pro Arg Glu Ala
130 135 140
Lys Val Gln Trp Lys Val Asp Asn Ala Leu Gln Ser Gly Asn Ser Gln
145 150 155 160
Glu Ser Val Thr Glu Gln Asp Ser Lys Asp Ser Thr Tyr Ser Leu Ser
165 170 175
Ser Thr Leu Thr Leu Ser Lys Ala Asp Tyr Glu Lys His Lys Val Tyr
180 185 190
Ala Cys Glu Val Thr His Gln Gly Leu Ser Ser Pro Val Thr Lys Ser
195 200 205
Phe Asn Arg Gly Glu Cys Ser Gly Gly Gly Ser Gly Gly Gly Ser Glu
210 215 220
Gly Gly Gly Ser Glu Gly Gly Gly Ser Glu Gly Gly Gly Ser Glu Gly
225 230 235 240
Gly Gly Ser Gly Gly Gly Ser Gly Glu Val Gln Leu Val Glu Ser Gly
245 250 255
Gly Gly Leu Val Gln Pro Gly Gly Ser Leu Arg Leu Ser Cys Ala Ala
260 265 270
Ser Gly Tyr Ser Phe Thr Gly Tyr Thr Met Asn Trp Val Arg Gln Ala
275 280 285
Pro Gly Lys Gly Leu Glu Trp Val Ala Leu Ile Asn Pro Tyr Lys Gly
290 295 300
Val Ser Thr Tyr Asn Gln Lys Phe Lys Asp Arg Phe Thr Ile Ser Val
305 310 315 320
Asp Lys Ser Lys Asn Thr Ala Tyr Leu Gln Met Asn Ser Leu Arg Ala
325 330 335
Glu Asp Thr Ala Val Tyr Tyr Cys Ala Arg Ser Gly Tyr Tyr Gly Asp
340 345 350
Ser Asp Trp Tyr Phe Asp Val Trp Gly Gln Gly Thr Leu Val Thr Val
355 360 365
Ser Ser Ala Ser Thr Lys Gly Pro Ser Val Phe Pro Leu Ala Pro Ser
370 375 380
Ser Lys Ser Thr Ser Gly Gly Thr Ala Ala Leu Gly Cys Leu Val Lys
385 390 395 400
Asp Tyr Phe Pro Glu Pro Val Thr Val Ser Trp Asn Ser Gly Ala Leu
405 410 415
Thr Ser Gly Val His Thr Phe Pro Ala Val Leu Gln Ser Ser Gly Leu
420 425 430
Tyr Ser Leu Ser Ser Val Val Thr Val Pro Ser Ser Ser Leu Gly Thr
435 440 445
Gln Thr Tyr Ile Cys Asn Val Asn His Lys Pro Ser Asn Thr Lys Val
450 455 460
Asp Lys Lys Val Glu Pro Lys Ser Cys Asp Gly Gly Gly Gly Ser Gly
465 470 475 480
Gly Gly Gly Ser Glu Val Gln Leu Leu Glu Ser Gly Gly Gly Leu Val
485 490 495
Gln Pro Gly Gly Ser Leu Arg Leu Ser Cys Ala Ala Ser Gly Phe Thr
500 505 510
Phe Ser Ser Tyr Ala Met Ser Trp Val Arg Gln Ala Pro Gly Lys Gly
515 520 525
Leu Glu Trp Val Ser Ala Ile Ser Gly Ser Gly Gly Ser Thr Tyr Tyr
530 535 540
Ala Asp Ser Val Lys Gly Arg Phe Thr Ile Ser Arg Asp Asn Ser Lys
545 550 555 560
Asn Thr Leu Tyr Leu Gln Met Asn Ser Leu Arg Ala Glu Asp Thr Ala
565 570 575
Val Tyr Tyr Cys Ala Lys Gly Trp Phe Gly Gly Phe Asn Tyr Trp Gly
580 585 590
Gln Gly Thr Leu Val Thr Val Ser Ser Ala Ser Thr Lys Gly Pro Ser
595 600 605
Val Phe Pro Leu Ala Pro Ser Ser Lys Ser Thr Ser Gly Gly Thr Ala
610 615 620
Ala Leu Gly Cys Leu Val Lys Asp Tyr Phe Pro Glu Pro Val Thr Val
625 630 635 640
Ser Trp Asn Ser Gly Ala Leu Thr Ser Gly Val His Thr Phe Pro Ala
645 650 655
Val Leu Gln Ser Ser Gly Leu Tyr Ser Leu Ser Ser Val Val Thr Val
660 665 670
Pro Ser Ser Ser Leu Gly Thr Gln Thr Tyr Ile Cys Asn Val Asn His
675 680 685
Lys Pro Ser Asn Thr Lys Val Asp Lys Lys Val Glu Pro Lys Ser Cys
690 695 700
Asp Lys Thr His Thr Cys Pro Pro Cys Pro Ala Pro Glu Ala Ala Gly
705 710 715 720
Gly Pro Ser Val Phe Leu Phe Pro Pro Lys Pro Lys Asp Thr Leu Met
725 730 735
Ile Ser Arg Thr Pro Glu Val Thr Cys Val Val Val Asp Val Ser His
740 745 750
Glu Asp Pro Glu Val Lys Phe Asn Trp Tyr Val Asp Gly Val Glu Val
755 760 765
His Asn Ala Lys Thr Lys Pro Arg Glu Glu Gln Tyr Asn Ser Thr Tyr
770 775 780
Arg Val Val Ser Val Leu Thr Val Leu His Gln Asp Trp Leu Asn Gly
785 790 795 800
Lys Glu Tyr Lys Cys Lys Val Ser Asn Lys Ala Leu Gly Ala Pro Ile
805 810 815
Glu Lys Thr Ile Ser Lys Ala Lys Gly Gln Pro Arg Glu Pro Gln Val
820 825 830
Tyr Thr Leu Pro Pro Cys Arg Asp Glu Leu Thr Lys Asn Gln Val Ser
835 840 845
Leu Trp Cys Leu Val Lys Gly Phe Tyr Pro Ser Asp Ile Ala Val Glu
850 855 860
Trp Glu Ser Asn Gly Gln Pro Glu Asn Asn Tyr Lys Thr Thr Pro Pro
865 870 875 880
Val Leu Asp Ser Asp Gly Ser Phe Phe Leu Tyr Ser Lys Leu Thr Val
885 890 895
Asp Lys Ser Arg Trp Gln Gln Gly Asn Val Phe Ser Cys Ser Val Met
900 905 910
His Glu Ala Leu His Asn His Tyr Thr Gln Lys Ser Leu Ser Leu Ser
915 920 925
Pro Gly Lys
930

SEQ ID NO: 58
<212> DNA
<223> V9 (scFab)-3F2 (VH-CH1)-Fc(knob) P329G LALA
gacatccaga tgacccagag cccctctagc ctgagcgcca gcgtgggcga cagagtgacc 60
atcacctgtc gggccagcca ggacatcaga aactacctga actggtatca gcagaagccc 120
ggcaaggccc ccaagctgct gatctactac acctctagac tggaaagcgg cgtgcccagc 180
cggtttagcg gcagcggctc cggcaccgac tacaccctga ccatcagcag cctgcagccc 240
gaggacttcg ccacctacta ctgccagcag ggcaacacac tcccctggac cttcggccag 300
ggcaccaagg tggagatcaa gcgtacggtg gccgctccca gcgtgttcat cttccccccc 360
agcgacgagc agctgaagtc cggcaccgcc agcgtcgtgt gcctgctgaa caacttctac 420
ccccgggagg ccaaggtgca gtggaaggtg gacaacgccc tgcagagcgg caacagccag 480
gaaagcgtca ccgagcagga cagcaaggac tccacctaca gcctgagcag caccctgacc 540
ctgagcaagg ccgactacga gaagcacaag gtgtacgcct gcgaagtgac ccaccagggc 600
ctgtccagcc ccgtgaccaa gagcttcaac cggggcgagt gcagcggcgg aggctctgga 660
ggcggctctg aaggcggagg aagtgagggc ggaggctcag aaggcggcgg aagcgaaggt 720
ggcggctctg gcggcggatc cggcgaggtg cagctggtcg agtccggcgg aggcctggtg 780
cagcctggcg gcagcctgag actgagctgc gccgccagcg gctacagctt caccggctac 840
accatgaact gggtccggca ggctcctggc aagggcctcg aatgggtggc cctgatcaac 900
ccctacaagg gcgtgagcac ctacaaccag aagttcaagg accggttcac catcagcgtg 960
gacaagagca agaacaccgc ctatctgcag atgaacagcc tgcgggccga ggacaccgcc 1020
gtgtactact gcgccagaag cggctactac ggcgacagcg actggtactt cgacgtgtgg 1080
ggccagggca cactggtcac cgtgtccagc gctagcacca agggcccctc cgtgttcccc 1140
ctggccccca gcagcaagag caccagcggc ggcacagccg ccctcggctg cctggtcaag 1200
gactacttcc ccgagcccgt gaccgtgtcc tggaacagcg gagccctgac ctccggcgtg 1260
cacaccttcc ccgccgtgct gcagagcagc ggcctgtaca gcctgtccag cgtggtcacc 1320
gtgccctcca gcagcctggg cacccagacc tacatctgca acgtgaacca caagcccagc 1380
aataccaagg tggacaagaa ggtggagccc aagagctgcg acggcggtgg tggctccgga 1440
ggaggaggca gcgaggtgca gctgctggaa tctggaggcg gcctggtgca gcctggcggc 1500
agcctgagac tgtcttgcgc cgccagcggc ttcaccttca gcagctacgc catgagctgg 1560
gtccgacagg ctcctggcaa gggactggaa tgggtgtccg ccatctccgg cagcggaggc 1620
agcacctact acgccgacag cgtgaagggc cggttcacca tcagcagaga caacagcaag 1680
aacaccctgt acctgcagat gaacagcctg cgggccgagg ataccgccgt gtattattgc 1740
gccaagggat ggttcggcgg cttcaactac tggggccagg gaaccctggt gacagtgtcc 1800
agcgccagca ccaagggccc ctccgtgttt cctctggccc ccagcagcaa gagcacctct 1860
ggcggaacag ccgccctggg ctgcctggtg aaagactact tccccgagcc cgtgaccgtg 1920
tcctggaact ctggcgccct gaccagcggc gtgcacacct ttccagccgt gctgcagagc 1980
agcggcctgt actccctgag cagcgtggtg acagtgccct ccagcagcct gggcacccag 2040
acctacatct gcaacgtgaa ccacaagccc agcaacacca aagtggacaa gaaggtggaa 2100
cccaagagct gcgacaaaac tcacacatgc ccaccgtgcc cagcacctga agctgcaggg 2160
ggaccgtcag tcttcctctt ccccccaaaa cccaaggaca ccctcatgat ctcccggacc 2220
cctgaggtca catgcgtggt ggtggacgtg agccacgaag accctgaggt caagttcaac 2280
tggtacgtgg acggcgtgga ggtgcataat gccaagacaa agccgcggga ggagcagtac 2340
aacagcacgt accgtgtggt cagcgtcctc accgtcctgc accaggactg gctgaatggc 2400
aaggagtaca agtgcaaggt ctccaacaaa gccctcggcg cccccatcga gaaaaccatc 2460
tccaaagcca aagggcagcc ccgagaacca caggtgtaca ccctgccccc atgccgggat 2520
gagctgacca agaaccaggt cagcctgtgg tgcctggtca aaggcttcta tcccagcgac 2580
atcgccgtgg agtgggagag caatgggcag ccggagaaca actacaagac cacgcctccc 2640
gtgctggact ccgacggctc cttcttcctc tacagcaagc tcaccgtgga caagagcagg 2700
tggcagcagg ggaacgtctt ctcatgctcc gtgatgcatg aggctctgca caaccactac 2760
acgcagaaga gcctctccct gtctccgggt aaatga 2796

SEQ ID NO: 59
<212> PRT
<223> 3F2 (VH-CH1)-Fc(hole) P329G LALA
Glu Val Gln Leu Leu Glu Ser Gly Gly Gly Leu Val Gln Pro Gly Gly
1 5 10 15
Ser Leu Arg Leu Ser Cys Ala Ala Ser Gly Phe Thr Phe Ser Ser Tyr
20 25 30
Ala Met Ser Trp Val Arg Gln Ala Pro Gly Lys Gly Leu Glu Trp Val
35 40 45
Ser Ala Ile Ser Gly Ser Gly Gly Ser Thr Tyr Tyr Ala Asp Ser Val
50 55 60
Lys Gly Arg Phe Thr Ile Ser Arg Asp Asn Ser Lys Asn Thr Leu Tyr
65 70 75 80
Leu Gln Met Asn Ser Leu Arg Ala Glu Asp Thr Ala Val Tyr Tyr Cys
85 90 95
Ala Lys Gly Trp Phe Gly Gly Phe Asn Tyr Trp Gly Gln Gly Thr Leu
100 105 110
Val Thr Val Ser Ser Ala Ser Thr Lys Gly Pro Ser Val Phe Pro Leu
115 120 125
Ala Pro Ser Ser Lys Ser Thr Ser Gly Gly Thr Ala Ala Leu Gly Cys
130 135 140
Leu Val Lys Asp Tyr Phe Pro Glu Pro Val Thr Val Ser Trp Asn Ser
145 150 155 160
Gly Ala Leu Thr Ser Gly Val His Thr Phe Pro Ala Val Leu Gln Ser
165 170 175
Ser Gly Leu Tyr Ser Leu Ser Ser Val Val Thr Val Pro Ser Ser Ser
180 185 190
Leu Gly Thr Gln Thr Tyr Ile Cys Asn Val Asn His Lys Pro Ser Asn
195 200 205
Thr Lys Val Asp Lys Lys Val Glu Pro Lys Ser Cys Asp Lys Thr His
210 215 220
Thr Cys Pro Pro Cys Pro Ala Pro Glu Ala Ala Gly Gly Pro Ser Val
225 230 235 240
Phe Leu Phe Pro Pro Lys Pro Lys Asp Thr Leu Met Ile Ser Arg Thr
245 250 255
Pro Glu Val Thr Cys Val Val Val Asp Val Ser His Glu Asp Pro Glu
260 265 270
Val Lys Phe Asn Trp Tyr Val Asp Gly Val Glu Val His Asn Ala Lys
275 280 285
Thr Lys Pro Arg Glu Glu Gln Tyr Asn Ser Thr Tyr Arg Val Val Ser
290 295 300
Val Leu Thr Val Leu His Gln Asp Trp Leu Asn Gly Lys Glu Tyr Lys
305 310 315 320
Cys Lys Val Ser Asn Lys Ala Leu Gly Ala Pro Ile Glu Lys Thr Ile
325 330 335
Ser Lys Ala Lys Gly Gln Pro Arg Glu Pro Gln Val Cys Thr Leu Pro
340 345 350
Pro Ser Arg Asp Glu Leu Thr Lys Asn Gln Val Ser Leu Ser Cys Ala
355 360 365
Val Lys Gly Phe Tyr Pro Ser Asp Ile Ala Val Glu Trp Glu Ser Asn
370 375 380
Gly Gln Pro Glu Asn Asn Tyr Lys Thr Thr Pro Pro Val Leu Asp Ser
385 390 395 400
Asp Gly Ser Phe Phe Leu Val Ser Lys Leu Thr Val Asp Lys Ser Arg
405 410 415
Trp Gln Gln Gly Asn Val Phe Ser Cys Ser Val Met His Glu Ala Leu
420 425 430
His Asn His Tyr Thr Gln Lys Ser Leu Ser Leu Ser Pro Gly Lys
435 440 445

SEQ ID NO: 60
<212> DNA
<223> 3F2 (VH-CH1)-Fc(hole) P329G LALA
gaggtgcagc tgctggaatc tggaggcggc ctggtgcagc ctggcggcag cctgagactg 60
tcttgcgccg ccagcggctt caccttcagc agctacgcca tgagctgggt ccgacaggct 120
cctggcaagg gactggaatg ggtgtccgcc atctccggca gcggaggcag cacctactac 180
gccgacagcg tgaagggccg gttcaccatc agcagagaca acagcaagaa caccctgtac 240
ctgcagatga acagcctgcg ggccgaggat accgccgtgt attattgcgc caagggatgg 300
ttcggcggct tcaactactg gggccaggga accctggtga cagtgtccag cgccagcacc 360
aagggcccct ccgtgtttcc tctggccccc agcagcaaga gcacctctgg cggaacagcc 420
gccctgggct gcctggtgaa agactacttc cccgagcccg tgaccgtgtc ctggaactct 480
ggcgccctga ccagcggcgt gcacaccttt ccagccgtgc tgcagagcag cggcctgtac 540
tccctgagca gcgtggtgac agtgccctcc agcagcctgg gcacccagac ctacatctgc 600
aacgtgaacc acaagcccag caacaccaaa gtggacaaga aggtggaacc caagagctgc 660
gacaaaactc acacatgccc accgtgccca gcacctgaag ctgcaggggg accgtcagtc 720
ttcctcttcc ccccaaaacc caaggacacc ctcatgatct cccggacccc tgaggtcaca 780
tgcgtggtgg tggacgtgag ccacgaagac cctgaggtca agttcaactg gtacgtggac 840
ggcgtggagg tgcataatgc caagacaaag ccgcgggagg agcagtacaa cagcacgtac 900
cgtgtggtca gcgtcctcac cgtcctgcac caggactggc tgaatggcaa ggagtacaag 960
tgcaaggtct ccaacaaagc cctcggcgcc cccatcgaga aaaccatctc caaagccaaa 1020
gggcagcccc gagaaccaca ggtgtgcacc ctgcccccat cccgggatga gctgaccaag 1080
aaccaggtca gcctctcgtg cgcagtcaaa ggcttctatc ccagcgacat cgccgtggag 1140
tgggagagca atgggcagcc ggagaacaac tacaagacca cgcctcccgt gctggactcc 1200
gacggctcct tcttcctcgt gagcaagctc accgtggaca agagcaggtg gcagcagggg 1260
aacgtcttct catgctccgt gatgcatgag gctctgcaca accactacac gcagaagagc 1320
ctctccctgt ctccgggtaa atga 1344

SEQ ID NO: 61
<212> PRT
<223> 3F2 (VL-CL)
Glu Ile Val Leu Thr Gln Ser Pro Gly Thr Leu Ser Leu Ser Pro Gly
1 5 10 15
Glu Arg Ala Thr Leu Ser Cys Arg Ala Ser Gln Ser Val Thr Ser Ser
20 25 30
Tyr Leu Ala Trp Tyr Gln Gln Lys Pro Gly Gln Ala Pro Arg Leu Leu
35 40 45
Ile Asn Val Gly Ser Arg Arg Ala Thr Gly Ile Pro Asp Arg Phe Ser
50 55 60
Gly Ser Gly Ser Gly Thr Asp Phe Thr Leu Thr Ile Ser Arg Leu Glu
65 70 75 80
Pro Glu Asp Phe Ala Val Tyr Tyr Cys Gln Gln Gly Ile Met Leu Pro
85 90 95
Pro Thr Phe Gly Gln Gly Thr Lys Val Glu Ile Lys Arg Thr Val Ala
100 105 110
Ala Pro Ser Val Phe Ile Phe Pro Pro Ser Asp Glu Gln Leu Lys Ser
115 120 125
Gly Thr Ala Ser Val Val Cys Leu Leu Asn Asn Phe Tyr Pro Arg Glu
130 135 140
Ala Lys Val Gln Trp Lys Val Asp Asn Ala Leu Gln Ser Gly Asn Ser
145 150 155 160
Gln Glu Ser Val Thr Glu Gln Asp Ser Lys Asp Ser Thr Tyr Ser Leu
165 170 175
Ser Ser Thr Leu Thr Leu Ser Lys Ala Asp Tyr Glu Lys His Lys Val
180 185 190
Tyr Ala Cys Glu Val Thr His Gln Gly Leu Ser Ser Pro Val Thr Lys
195 200 205
Ser Phe Asn Arg Gly Glu Cys
210 215

SEQ ID NO: 62
<212> DNA
<223> 3F2 (VL-CL)
gagatcgtgc tgacccagtc tcccggcacc ctgagcctga gccctggcga gagagccacc 60
ctgagctgca gagccagcca gagcgtgacc agcagctacc tggcctggta tcagcagaag 120
cccggccagg cccccagact gctgatcaac gtgggcagca gacgggccac cggcatcccc 180
gatagattca gcggcagcgg ctccggcacc gacttcaccc tgaccatcag ccggctggaa 240
cccgaggact tcgccgtgta ctactgccag cagggcatca tgctgccccc caccttcggc 300
cagggcacca aggtggaaat caagcggacc gtggccgctc ccagcgtgtt catcttccca 360
cccagcgacg agcagctgaa gtccggcaca gccagcgtgg tgtgcctgct gaacaacttc 420
tacccccgcg aggccaaggt gcagtggaag gtggacaacg ccctgcagag cggcaacagc 480
caggaatccg tgaccgagca ggacagcaag gactccacct acagcctgag cagcaccctg 540
accctgagca aggccgacta cgagaagcac aaggtgtacg cctgcgaagt gacccaccag 600
ggcctgtcca gccccgtgac caagagcttc aaccggggcg agtgctga 648

SEQ ID NO: 63
<212> PRT
<223> CH1A1A (VH-CH1)- V9 (VL-CH1)-Fc(knob) P329G LALA
Gln Val Gln Leu Val Gln Ser Gly Ala Glu Val Lys Lys Pro Gly Ala
1 5 10 15
Ser Val Lys Val Ser Cys Lys Ala Ser Gly Tyr Thr Phe Thr Glu Phe
20 25 30
Gly Met Asn Trp Val Arg Gln Ala Pro Gly Gln Gly Leu Glu Trp Met
35 40 45
Gly Trp Ile Asn Thr Lys Thr Gly Glu Ala Thr Tyr Val Glu Glu Phe
50 55 60
Lys Gly Arg Val Thr Phe Thr Thr Asp Thr Ser Thr Ser Thr Ala Tyr
65 70 75 80
Met Glu Leu Arg Ser Leu Arg Ser Asp Asp Thr Ala Val Tyr Tyr Cys
85 90 95
Ala Arg Trp Asp Phe Ala Tyr Tyr Val Glu Ala Met Asp Tyr Trp Gly
100 105 110
Gln Gly Thr Thr Val Thr Val Ser Ser Ala Ser Thr Lys Gly Pro Ser
115 120 125
Val Phe Pro Leu Ala Pro Ser Ser Lys Ser Thr Ser Gly Gly Thr Ala
130 135 140
Ala Leu Gly Cys Leu Val Lys Asp Tyr Phe Pro Glu Pro Val Thr Val
145 150 155 160
Ser Trp Asn Ser Gly Ala Leu Thr Ser Gly Val His Thr Phe Pro Ala
165 170 175
Val Leu Gln Ser Ser Gly Leu Tyr Ser Leu Ser Ser Val Val Thr Val
180 185 190
Pro Ser Ser Ser Leu Gly Thr Gln Thr Tyr Ile Cys Asn Val Asn His
195 200 205
Lys Pro Ser Asn Thr Lys Val Asp Lys Lys Val Glu Pro Lys Ser Cys
210 215 220
Gly Gly Gly Gly Ser Gly Gly Gly Gly Ser Asp Ile Gln Met Thr Gln
225 230 235 240
Ser Pro Ser Ser Leu Ser Ala Ser Val Gly Asp Arg Val Thr Ile Thr
245 250 255
Cys Arg Ala Ser Gln Asp Ile Arg Asn Tyr Leu Asn Trp Tyr Gln Gln
260 265 270
Lys Pro Gly Lys Ala Pro Lys Leu Leu Ile Tyr Tyr Thr Ser Arg Leu
275 280 285
Glu Ser Gly Val Pro Ser Arg Phe Ser Gly Ser Gly Ser Gly Thr Asp
290 295 300
Tyr Thr Leu Thr Ile Ser Ser Leu Gln Pro Glu Asp Phe Ala Thr Tyr
305 310 315 320
Tyr Cys Gln Gln Gly Asn Thr Leu Pro Trp Thr Phe Gly Gln Gly Thr
325 330 335
Lys Val Glu Ile Lys Ser Ser Ala Ser Thr Lys Gly Pro Ser Val Phe
340 345 350
Pro Leu Ala Pro Ser Ser Lys Ser Thr Ser Gly Gly Thr Ala Ala Leu
355 360 365
Gly Cys Leu Val Lys Asp Tyr Phe Pro Glu Pro Val Thr Val Ser Trp
370 375 380
Asn Ser Gly Ala Leu Thr Ser Gly Val His Thr Phe Pro Ala Val Leu
385 390 395 400
Gln Ser Ser Gly Leu Tyr Ser Leu Ser Ser Val Val Thr Val Pro Ser
405 410 415
Ser Ser Leu Gly Thr Gln Thr Tyr Ile Cys Asn Val Asn His Lys Pro
420 425 430
Ser Asn Thr Lys Val Asp Lys Lys Val Glu Pro Lys Ser Cys Asp Lys
435 440 445
Thr His Thr Cys Pro Pro Cys Pro Ala Pro Glu Ala Ala Gly Gly Pro
450 455 460
Ser Val Phe Leu Phe Pro Pro Lys Pro Lys Asp Thr Leu Met Ile Ser
465 470 475 480
Arg Thr Pro Glu Val Thr Cys Val Val Val Asp Val Ser His Glu Asp
485 490 495
Pro Glu Val Lys Phe Asn Trp Tyr Val Asp Gly Val Glu Val His Asn
500 505 510
Ala Lys Thr Lys Pro Arg Glu Glu Gln Tyr Asn Ser Thr Tyr Arg Val
515 520 525
Val Ser Val Leu Thr Val Leu His Gln Asp Trp Leu Asn Gly Lys Glu
530 535 540
Tyr Lys Cys Lys Val Ser Asn Lys Ala Leu Gly Ala Pro Ile Glu Lys
545 550 555 560
Thr Ile Ser Lys Ala Lys Gly Gln Pro Arg Glu Pro Gln Val Tyr Thr
565 570 575
Leu Pro Pro Cys Arg Asp Glu Leu Thr Lys Asn Gln Val Ser Leu Trp
580 585 590
Cys Leu Val Lys Gly Phe Tyr Pro Ser Asp Ile Ala Val Glu Trp Glu
595 600 605
Ser Asn Gly Gln Pro Glu Asn Asn Tyr Lys Thr Thr Pro Pro Val Leu
610 615 620
Asp Ser Asp Gly Ser Phe Phe Leu Tyr Ser Lys Leu Thr Val Asp Lys
625 630 635 640
Ser Arg Trp Gln Gln Gly Asn Val Phe Ser Cys Ser Val Met His Glu
645 650 655
Ala Leu His Asn His Tyr Thr Gln Lys Ser Leu Ser Leu Ser Pro Gly
660 665 670
Lys

SEQ ID NO: 64
<212> DNA
<223> CH1A1A (VH-CH1)- V9 (VL-CH1)-Fc(knob) P329G LALA
caggtgcagc tggtgcagtc tggcgccgaa gtgaagaaac ctggcgccag cgtgaaggtg 60
tcctgcaagg ccagcggcta caccttcacc gagttcggca tgaactgggt ccgacaggcc 120
cctggacagg gcctggaatg gatgggctgg atcaacacca agaccggcga ggccacctac 180
gtggaagagt tcaagggcag agtgaccttc accaccgaca ccagcaccag caccgcctac 240
atggaactgc ggagcctgag aagcgacgac accgccgtgt actactgcgc cagatgggac 300
ttcgcctact atgtggaagc catggactac tggggccagg gcaccaccgt gaccgtgtct 360
agtgctagca caaagggccc cagcgtgttc cctctggccc ctagcagcaa gagcacatct 420
ggcggaacag ccgccctggg ctgcctggtc aaggactact ttcccgagcc cgtgacagtg 480
tcctggaact ctggcgccct gacaagcggc gtgcacacct ttccagccgt gctgcagagc 540
agcggcctgt actctctgag cagcgtggtc accgtgccta gctctagcct gggcacccag 600
acctacatct gcaacgtgaa ccacaagccc agcaacacca aggtggacaa gaaggtggaa 660
cccaagagct gcggcggagg cggatccgga ggcggaggat ctgatatcca gatgacccag 720
agccccagca gcctgtctgc cagcgtgggc gacagagtga ccattacctg cagagccagc 780
caggacatca gaaactacct gaactggtat cagcagaagc ccggcaaggc ccccaagctg 840
ctgatctact acaccagcag actggaatcc ggcgtgccca gcagattttc cggcagcggc 900
tctggcaccg actacaccct gacaatcagc agcctgcagc ccgaggactt cgccacctac 960
tactgccagc agggcaacac cctgccctgg acatttggac agggcacaaa ggtggaaatc 1020
aagagcagcg cctccaccaa gggcccttcc gtgtttccac tggcccccag ctctaagagc 1080
accagcggag gaacagctgc tctgggatgt ctcgtgaagg attacttccc cgaacctgtg 1140
accgtcagct ggaacagcgg cgctctgaca tctggggtgc acacattccc cgctgtcctg 1200
cagtcctccg gcctgtacag tctgtccagc gtcgtgacag tgcctagcag ctccctggga 1260
acacagacat atatctgtaa tgtcaatcac aagccctcta ataccaaggt cgacaaaaaa 1320
gtcgagccca agtcctgcga caagacccac acctgtcccc cttgtcctgc ccctgaagct 1380
gctggcggcc cttctgtgtt cctgttcccc ccaaagccca aggacaccct gatgatcagc 1440
cggacccccg aagtgacctg cgtggtggtg gatgtgtccc acgaggaccc tgaagtgaag 1500
ttcaattggt acgtggacgg cgtggaagtg cacaacgcca agacaaagcc gcgggaggag 1560
cagtacaaca gcacgtaccg tgtggtcagc gtcctcaccg tcctgcacca ggactggctg 1620
aatggcaagg agtacaagtg caaggtctcc aacaaagccc tcggcgcccc catcgagaaa 1680
accatctcca aagccaaagg gcagccccga gaaccacagg tgtacaccct gcccccatgc 1740
cgggatgagc tgaccaagaa ccaggtcagc ctgtggtgcc tggtcaaagg cttctatccc 1800
agcgacatcg ccgtggagtg ggagagcaat gggcagccgg agaacaacta caagaccacg 1860
cctcccgtgc tggactccga cggctccttc ttcctctaca gcaagctcac cgtggacaag 1920
agcaggtggc agcaggggaa cgtcttctca tgctccgtga tgcatgaggc tctgcacaac 1980
cactacacgc agaagagcct ctccctgtct ccgggtaaat ga 2022

SEQ ID NO: 65
<212> PRT
<223> CH1A1A (VH-CH1)-Fc(hole) P329G LALA
Gln Val Gln Leu Val Gln Ser Gly Ala Glu Val Lys Lys Pro Gly Ala
1 5 10 15
Ser Val Lys Val Ser Cys Lys Ala Ser Gly Tyr Thr Phe Thr Glu Phe
20 25 30
Gly Met Asn Trp Val Arg Gln Ala Pro Gly Gln Gly Leu Glu Trp Met
35 40 45
Gly Trp Ile Asn Thr Lys Thr Gly Glu Ala Thr Tyr Val Glu Glu Phe
50 55 60
Lys Gly Arg Val Thr Phe Thr Thr Asp Thr Ser Thr Ser Thr Ala Tyr
65 70 75 80
Met Glu Leu Arg Ser Leu Arg Ser Asp Asp Thr Ala Val Tyr Tyr Cys
85 90 95
Ala Arg Trp Asp Phe Ala Tyr Tyr Val Glu Ala Met Asp Tyr Trp Gly
100 105 110
Gln Gly Thr Thr Val Thr Val Ser Ser Ala Ser Thr Lys Gly Pro Ser
115 120 125
Val Phe Pro Leu Ala Pro Ser Ser Lys Ser Thr Ser Gly Gly Thr Ala
130 135 140
Ala Leu Gly Cys Leu Val Lys Asp Tyr Phe Pro Glu Pro Val Thr Val
145 150 155 160
Ser Trp Asn Ser Gly Ala Leu Thr Ser Gly Val His Thr Phe Pro Ala
165 170 175
Val Leu Gln Ser Ser Gly Leu Tyr Ser Leu Ser Ser Val Val Thr Val
180 185 190
Pro Ser Ser Ser Leu Gly Thr Gln Thr Tyr Ile Cys Asn Val Asn His
195 200 205
Lys Pro Ser Asn Thr Lys Val Asp Lys Lys Val Glu Pro Lys Ser Cys
210 215 220
Asp Lys Thr His Thr Cys Pro Pro Cys Pro Ala Pro Glu Ala Ala Gly
225 230 235 240
Gly Pro Ser Val Phe Leu Phe Pro Pro Lys Pro Lys Asp Thr Leu Met
245 250 255
Ile Ser Arg Thr Pro Glu Val Thr Cys Val Val Val Asp Val Ser His
260 265 270
Glu Asp Pro Glu Val Lys Phe Asn Trp Tyr Val Asp Gly Val Glu Val
275 280 285
His Asn Ala Lys Thr Lys Pro Arg Glu Glu Gln Tyr Asn Ser Thr Tyr
290 295 300
Arg Val Val Ser Val Leu Thr Val Leu His Gln Asp Trp Leu Asn Gly
305 310 315 320
Lys Glu Tyr Lys Cys Lys Val Ser Asn Lys Ala Leu Gly Ala Pro Ile
325 330 335
Glu Lys Thr Ile Ser Lys Ala Lys Gly Gln Pro Arg Glu Pro Gln Val
340 345 350
Cys Thr Leu Pro Pro Ser Arg Asp Glu Leu Thr Lys Asn Gln Val Ser
355 360 365
Leu Ser Cys Ala Val Lys Gly Phe Tyr Pro Ser Asp Ile Ala Val Glu
370 375 380
Trp Glu Ser Asn Gly Gln Pro Glu Asn Asn Tyr Lys Thr Thr Pro Pro
385 390 395 400
Val Leu Asp Ser Asp Gly Ser Phe Phe Leu Val Ser Lys Leu Thr Val
405 410 415
Asp Lys Ser Arg Trp Gln Gln Gly Asn Val Phe Ser Cys Ser Val Met
420 425 430
His Glu Ala Leu His Asn His Tyr Thr Gln Lys Ser Leu Ser Leu Ser
435 440 445
Pro Gly Lys
450

SEQ ID NO: 66
<212> DNA
<223> CH1A1A (VH-CH1)-Fc(hole) P329G LALA
caggtgcagc tggtgcagtc tggcgccgaa gtgaagaaac ctggagctag tgtgaaggtg 60
tcctgcaagg ccagcggcta caccttcacc gagttcggca tgaactgggt ccgacaggct 120
ccaggccagg gcctcgaatg gatgggctgg atcaacacca agaccggcga ggccacctac 180
gtggaagagt tcaagggcag agtgaccttc accacggaca ccagcaccag caccgcctac 240
atggaactgc ggagcctgag aagcgacgac accgccgtgt actactgcgc cagatgggac 300
ttcgcctatt acgtggaagc catggactac tggggccagg gcaccaccgt gaccgtgtct 360
agcgctagca ccaagggccc ctccgtgttc cccctggccc ccagcagcaa gagcaccagc 420
ggcggcacag ccgctctggg ctgcctggtc aaggactact tccccgagcc cgtgaccgtg 480
tcctggaaca gcggagccct gacctccggc gtgcacacct tccccgccgt gctgcagagt 540
tctggcctgt atagcctgag cagcgtggtc accgtgcctt ctagcagcct gggcacccag 600
acctacatct gcaacgtgaa ccacaagccc agcaacacca aggtggacaa gaaggtggag 660
cccaagagct gcgacaaaac tcacacatgc ccaccgtgcc cagcacctga agctgcaggg 720
ggaccgtcag tcttcctctt ccccccaaaa cccaaggaca ccctcatgat ctcccggacc 780
cctgaggtca catgcgtggt ggtggacgtg agccacgaag accctgaggt caagttcaac 840
tggtacgtgg acggcgtgga ggtgcataat gccaagacaa agccgcggga ggagcagtac 900
aacagcacgt accgtgtggt cagcgtcctc accgtcctgc accaggactg gctgaatggc 960
aaggagtaca agtgcaaggt ctccaacaaa gccctcggcg cccccatcga gaaaaccatc 1020
tccaaagcca aagggcagcc ccgagaacca caggtgtgca ccctgccccc atcccgggat 1080
gagctgacca agaaccaggt cagcctctcg tgcgcagtca aaggcttcta tcccagcgac 1140
atcgccgtgg agtgggagag caatgggcag ccggagaaca actacaagac cacgcctccc 1200
gtgctggact ccgacggctc cttcttcctc gtgagcaagc tcaccgtgga caagagcagg 1260
tggcagcagg ggaacgtctt ctcatgctcc gtgatgcatg aggctctgca caaccactac 1320
acgcagaaga gcctctccct gtctccgggt aaatga 1356

SEQ ID NO: 67
<212> PRT
<223> CH1A1A (VL-CL)
Asp Ile Gln Met Thr Gln Ser Pro Ser Ser Leu Ser Ala Ser Val Gly
1 5 10 15
Asp Arg Val Thr Ile Thr Cys Lys Ala Ser Ala Ala Val Gly Thr Tyr
20 25 30
Val Ala Trp Tyr Gln Gln Lys Pro Gly Lys Ala Pro Lys Leu Leu Ile
35 40 45
Tyr Ser Ala Ser Tyr Arg Lys Arg Gly Val Pro Ser Arg Phe Ser Gly
50 55 60
Ser Gly Ser Gly Thr Asp Phe Thr Leu Thr Ile Ser Ser Leu Gln Pro
65 70 75 80
Glu Asp Phe Ala Thr Tyr Tyr Cys His Gln Tyr Tyr Thr Tyr Pro Leu
85 90 95
Phe Thr Phe Gly Gln Gly Thr Lys Leu Glu Ile Lys Arg Thr Val Ala
100 105 110
Ala Pro Ser Val Phe Ile Phe Pro Pro Ser Asp Glu Gln Leu Lys Ser
115 120 125
Gly Thr Ala Ser Val Val Cys Leu Leu Asn Asn Phe Tyr Pro Arg Glu
130 135 140
Ala Lys Val Gln Trp Lys Val Asp Asn Ala Leu Gln Ser Gly Asn Ser
145 150 155 160
Gln Glu Ser Val Thr Glu Gln Asp Ser Lys Asp Ser Thr Tyr Ser Leu
165 170 175
Ser Ser Thr Leu Thr Leu Ser Lys Ala Asp Tyr Glu Lys His Lys Val
180 185 190
Tyr Ala Cys Glu Val Thr His Gln Gly Leu Ser Ser Pro Val Thr Lys
195 200 205
Ser Phe Asn Arg Gly Glu Cys
210 215

SEQ ID NO: 68<212> DNA
<223> CH1A1A (VL-CL)
gatatccaga tgacccagtc tccatcctcc ctgtctgcat ctgtgggaga cagagtcacc 60
atcacttgca aggccagtgc ggctgtgggt acgtatgttg cgtggtatca gcagaaacca 120
gggaaagcac ctaagctcct gatctattcg gcatcctacc gcaaaagggg agtcccatca 180
aggttcagtg gcagtggatc tgggacagat ttcactctca ccatcagcag tctgcaacct 240
gaagatttcg caacttacta ctgtcaccaa tattacacct atcctctatt cacgtttggc 300
cagggcacca agctcgagat caagcgtacg gtggctgcac catctgtctt catcttcccg 360
ccatctgatg agcagttgaa atctggaact gcctctgttg tgtgcctgct gaataacttc 420
tatcccagag aggccaaagt acagtggaag gtggataacg ccctccaatc gggtaactcc 480
caggagagtg tcacagagca ggacagcaag gacagcacct acagcctcag cagcaccctg 540
acgctgagca aagcagacta cgagaaacac aaagtctacg cctgcgaagt cacccatcag 600
ggcctgagct cgcccgtcac aaagagcttc aacaggggag agtgttag 648

SEQ ID NO: 69
<212> PRT
<223> LC007 HCDR1
Gly Tyr Ser Ile Thr Ser Gly Tyr Tyr Trp Asn
1 5 10

SEQ ID NO: 70
<212> DNA
<223> LC007 HCDR1
ggctactcca tcaccagtgg ttattactgg aac 33

SEQ ID NO: 71
<212> PRT
<223> LC007 HCDR2
Tyr Ile Thr Tyr Asp Gly Ser Asn Asn Tyr Asn Pro Ser Leu Lys Asn
1 5 10 15

SEQ ID NO: 72
<212> DNA
<223> LC007 HCDR2
tacataacct acgacggtag caataactac aacccatctc tcaaaaat 48

SEQ ID NO: 73
<212> PRT
<223> LC007 HCDR3
Phe Asp Tyr
1

SEQ ID NO: 74
<212> DNA
<223> LC007 HCDR3
tttgactac 9

SEQ ID NO: 75
<212> PRT
<223> LC007 VH
Glu Val Gln Leu Gln Glu Ser Gly Pro Gly Leu Val Lys Pro Ser Gln
1 5 10 15
Ser Leu Ser Leu Thr Cys Ser Val Thr Gly Tyr Ser Ile Thr Ser Gly
20 25 30
Tyr Tyr Trp Asn Trp Ile Arg Gln Phe Pro Gly Asn Lys Leu Glu Trp
35 40 45
Met Gly Tyr Ile Thr Tyr Asp Gly Ser Asn Asn Tyr Asn Pro Ser Leu
50 55 60
Lys Asn Arg Ile Ser Ile Thr Arg Asp Thr Ser Lys Asn Gln Phe Phe
65 70 75 80
Leu Lys Leu Asn Ser Val Thr Thr Glu Asp Thr Ala Thr Tyr Tyr Cys
85 90 95
Ala Asp Phe Asp Tyr Trp Gly Gln Gly Thr Thr Leu Thr Val Ser Ser
100 105 110

SEQ ID NO: 76
<212> DNA
<223> LC007 VH
gaggtccagc tgcaggagtc aggacctggc ctcgtgaaac cttctcagtc tctgtctctc 60
acctgctctg tcactggcta ctccatcacc agtggttatt actggaactg gatccggcag 120
tttccaggaa acaagctgga atggatgggc tacataacct acgacggtag caataactac 180
aacccatctc tcaaaaatcg aatctccatc actcgtgaca catctaagaa ccagtttttc 240
ctgaagttga attctgtgac tactgaggac acagctacat attactgtgc ggactttgac 300
tactggggcc aaggcaccac tctcacagtc tcctca 336

SEQ ID NO: 77
<212> PRT
<223> LC007 LCDR1
Ser Ala Ser Gln Gly Ile Arg Asn Tyr Leu Asn
1 5 10

SEQ ID NO: 78
<212> DNA
<223> LC007 LCDR1
agtgcaagtc agggcattag aaattattta aac 33

SEQ ID NO: 79
<212> PRT
<223> LC007 LCDR2
Tyr Thr Ser Ser Leu His Ser
1 5

SEQ ID NO: 80
<212> DNA
<223> LC007 LCDR2
tacacatcaa gtttacactc a 21

SEQ ID NO: 81
<212> PRT
<223> LC007 LCDR3
Gln Gln Tyr Ser Lys Leu Pro Trp Thr
1 5

SEQ ID NO: 82
<212> DNA
<223> LC007 LCDR3
cagcagtata gtaagcttcc ttggacg 27

SEQ ID NO: 83
<212> PRT
<223> LC007 VL
Asp Ile Val Leu Thr Gln Ser Pro Ser Ser Leu Ser Ala Ser Leu Gly
1 5 =10 15
Asp Arg Val Thr Ile Ser Cys Ser Ala Ser Gln Gly Ile Arg Asn Tyr
20 25 30
Leu Asn Trp Tyr Gln Gln Arg Pro Asp Gly Thr Val Lys Leu Leu Ile
35 40 45
Tyr Tyr Thr Ser Ser Leu His Ser Gly Val Pro Ser Arg Phe Ser Gly
50 55 60
Ser Gly Ser Gly Thr Asp Tyr Ser Leu Thr Ile Ser Asn Leu Glu Pro
65 70 75 80
Glu Asp Ile Ala Thr Tyr Tyr Cys Gln Gln Tyr Ser Lys Leu Pro Trp
85 90 95
Thr Phe Gly Gly Gly Thr Lys Leu Glu Ile Lys
100 105

SEQ ID NO: 84
<212> DNA
<223> LC007 VL
gatattgtgc tcacacagtc tccatcctcc ctgtctgcct ctctgggaga cagagtcacc 60
atcagttgca gtgcaagtca gggcattaga aattatttaa actggtatca gcagagacca 120
gatggaactg ttaaactcct gatctattac acatcaagtt tacactcagg agtcccatca 180
aggttcagtg gcagtgggtc tgggacagat tattctctca ccatcagcaa cctggaacct 240
gaagatattg ccacttacta ttgtcagcag tatagtaagc ttccttggac gttcggtgga 300
ggcaccaagc tggaaatcaa a 321

SEQ ID NO: 85
<212> PRT
<223> GA201 HCDR1
Asp Tyr Lys Ile His
1 5

SEQ ID NO: 86
<212> DNA
<223> GA201 HCDR1
gactacaaga tacac 15

SEQ ID NO: 87
<212> PRT
<223> GA201 HCDR2
Tyr Phe Asn Pro Asn Ser Gly Tyr Ser Thr Tyr Ala Gln Lys Phe Gln
1 5 10 15
Gly

SEQ ID NO: 88
<212> DNA
<223> GA201 HCDR2
tatttcaacc ctaacagcgg ttatagtacc tacgcacaga agttccaggg c 51

SEQ ID NO: 89
<212> PRT
<223> GA201 HCDR3
Leu Ser Pro Gly Gly Tyr Tyr Val Met Asp Ala
1 5 10

SEQ ID NO: 90
<212> DNA
<223> GA201 HCDR3
ctatccccag gcggttacta tgttatggat gcc 33

SEQ ID NO: 91
<212> PRT
<223> GA201 VH
Gln Val Gln Leu Val Gln Ser Gly Ala Glu Val Lys Lys Pro Gly Ser
1 5 10 15
Ser Val Lys Val Ser Cys Lys Ala Ser Gly Phe Thr Phe Thr Asp Tyr
20 25 30
Lys Ile His Trp Val Arg Gln Ala Pro Gly Gln Gly Leu Glu Trp Met
35 40 45
Gly Tyr Phe Asn Pro Asn Ser Gly Tyr Ser Thr Tyr Ala Gln Lys Phe
50 55 60
Gln Gly Arg Val Thr Ile Thr Ala Asp Lys Ser Thr Ser Thr Ala Tyr
65 70 75 80
Met Glu Leu Ser Ser Leu Arg Ser Glu Asp Thr Ala Val Tyr Tyr Cys
85 90 95
Ala Arg Leu Ser Pro Gly Gly Tyr Tyr Val Met Asp Ala Trp Gly Gln
100 105 110
Gly Thr Thr Val Thr Val Ser Ser
115 120

SEQ ID NO: 92
<212> DNA
<223> GA201 VH
caggtgcagc tggtgcagtc tggggctgag gtgaagaagc ctgggtcctc ggtgaaggtc 60
tcctgcaagg cctctggttt cacattcact gactacaaga tacactgggt gcgacaggcc 120
cctggacaag ggctcgagtg gatgggatat ttcaacccta acagcggtta tagtacctac 180
gcacagaagt tccagggcag ggtcaccatt accgcggaca aatccacgag cacagcctac 240
atggagctga gcagcctgag atctgaggac acggccgtgt attactgtgc gagactatcc 300
ccaggcggtt actatgttat ggatgcctgg ggccaaggga ccaccgtgac cgtctcctca 360

SEQ ID NO: 93
<212> PRT
<223> GA201 LCDR1
Arg Ala Ser Gln Gly Ile Asn Asn Tyr Leu Asn
1 5 10

SEQ ID NO: 94
<212> DNA
<223> GA201 LCDR1
cgggcaagtc agggcattaa caattactta aat 33

SEQ ID NO: 95
<212> PRT
<223> GA201 LCDR2
Asn Thr Asn Asn Leu Gln Thr
1 5

SEQ ID NO: 96
<212> DNA
<223> GA201 LCDR2
aataccaaca acttgcagac a 21

SEQ ID NO: 97
<212> PRT
<223> GA201 LCDR3
Leu Gln His Asn Ser Phe Pro Thr
1 5

SEQ ID NO: 98
<212> DNA
<223> GA201 LCDR3
ttgcagcata atagttttcc cacg 24

SEQ ID NO: 99
<212> PRT
<223> GA201 VL
Asp Ile Gln Met Thr Gln Ser Pro Ser Ser Leu Ser Ala Ser Val Gly
1 5 10 15
Asp Arg Val Thr Ile Thr Cys Arg Ala Ser Gln Gly Ile Asn Asn Tyr
20 25 30
Leu Asn Trp Tyr Gln Gln Lys Pro Gly Lys Ala Pro Lys Arg Leu Ile
35 40 45
Tyr Asn Thr Asn Asn Leu Gln Thr Gly Val Pro Ser Arg Phe Ser Gly
50 55 60
Ser Gly Ser Gly Thr Glu Phe Thr Leu Thr Ile Ser Ser Leu Gln Pro
65 70 75 80
Glu Asp Phe Ala Thr Tyr Tyr Cys Leu Gln His Asn Ser Phe Pro Thr
85 90 95
Phe Gly Gln Gly Thr Lys Leu Glu Ile Lys
100 105

SEQ ID NO: 100
<212> DNA
<223> GA201 VL
gatatccaga tgacccagtc tccatcctcc ctgtctgcat ctgtcggaga ccgggtcacc 60
atcacctgcc gggcaagtca gggcattaac aattacttaa attggtacca gcagaagcca 120
gggaaagccc ctaagcgcct gatctataat accaacaact tgcagacagg cgtcccatca 180
aggttcagcg gcagtggatc cgggacagaa ttcactctca ccatcagcag cctgcagcct 240
gaagattttg ccacctatta ctgcttgcag cataatagtt ttcccacgtt tggccagggc 300
accaagctcg agatcaag 318

SEQ ID NO: 101
<212> PRT
<223> 3F2 HCDR1
Ser Tyr Ala Met Ser
1 5

SEQ ID NO: 102
<212> DNA
<223> 3F2 HCDR1
agctacgcca tgagc 15

SEQ ID NO: 103
<212> PRT
<223> 3F2 HCDR2
Ala Ile Ser Gly Ser Gly Gly Ser Thr Tyr Tyr Ala Asp Ser Val Lys
1 5 10 15

SEQ ID NO: 104
<212> DNA
<223> 3F2 HCDR2
gccatctccg gcagcggagg cagcacctac tacgccgaca gcgtgaag 48

SEQ ID NO: 105
<212> PRT
<223> 3F2 HCDR3
Tyr Cys Ala Lys Gly Trp Phe Gly
1 5

SEQ ID NO: 106
<212> DNA
<223> 3F2 HCDR3
tattgcgcca agggatggtt cggc 24

SEQ ID NO: 107
<212> PRT
<223> 3F2 VH
Glu Val Gln Leu Leu Glu Ser Gly Gly Gly Leu Val Gln Pro Gly Gly
1 5 10 15
Ser Leu Arg Leu Ser Cys Ala Ala Ser Gly Phe Thr Phe Ser Ser Tyr
20 25 30
Ala Met Ser Trp Val Arg Gln Ala Pro Gly Lys Gly Leu Glu Trp Val
35 40 45
Ser Ala Ile Ser Gly Ser Gly Gly Ser Thr Tyr Tyr Ala Asp Ser Val
50 55 60
Lys Gly Arg Phe Thr Ile Ser Arg Asp Asn Ser Lys Asn Thr Leu Tyr
65 70 75 80
Leu Gln Met Asn Ser Leu Arg Ala Glu Asp Thr Ala Val Tyr Tyr Cys
85 90 95
Ala Lys Gly Trp Phe Gly Gly Phe Asn Tyr Trp Gly Gln Gly Thr Leu
100 105 110
Val Thr Val Ser Ser
115

SEQ ID NO: 108
<212> DNA
<223> 3F2 VH
gaggtgcagc tgctggaatc tggaggcggc ctggtgcagc ctggcggcag cctgagactg 60
tcttgcgccg ccagcggctt caccttcagc agctacgcca tgagctgggt ccgacaggct 120
cctggcaagg gactggaatg ggtgtccgcc atctccggca gcggaggcag cacctactac 180
gccgacagcg tgaagggccg gttcaccatc agcagagaca acagcaagaa caccctgtac 240
ctgcagatga acagcctgcg ggccgaggat accgccgtgt attattgcgc caagggatgg 300
ttcggcggct tcaactactg gggccaggga accctggtga cagtgtccag c 351

SEQ ID NO: 109
<212> PRT
<223> 3F2 LCDR1
Arg Ala Ser Gln Ser Val Thr Ser Ser Tyr Leu
1 5 10

SEQ ID NO: 110
<212> DNA
<223> 3F2 LCDR1
agagccagcc agagcgtgac cagcagctac ctg 33

SEQ ID NO: 111
<212> PRT
<223> 3F2 LCDR2
Asn Val Gly Ser Arg Arg Ala
1 5

SEQ ID NO: 112
<212> DNA
<223> 3F2 LCDR2
aacgtgggca gcagacgggc c 21

SEQ ID NO: 113
<212> PRT
<223> 3F2 LCDR3
Cys Gln Gln Gly Ile Met Leu Pro Pro
1 5

SEQ ID NO: 114
<212> DNA
<223> 3F2 LCDR3
tgccagcagg gcatcatgct gcccccc 27

SEQ ID NO: 115
<212> PRT
<223> 3F2 VL
Glu Ile Val Leu Thr Gln Ser Pro Gly Thr Leu Ser Leu Ser Pro Gly
1 5 10 15
Glu Arg Ala Thr Leu Ser Cys Arg Ala Ser Gln Ser Val Thr Ser Ser
20 25 30
Tyr Leu Ala Trp Tyr Gln Gln Lys Pro Gly Gln Ala Pro Arg Leu Leu
35 40 45
Ile Asn Val Gly Ser Arg Arg Ala Thr Gly Ile Pro Asp Arg Phe Ser
50 55 60
Gly Ser Gly Ser Gly Thr Asp Phe Thr Leu Thr Ile Ser Arg Leu Glu
65 70 75 80
Pro Glu Asp Phe Ala Val Tyr Tyr Cys Gln Gln Gly Ile Met Leu Pro
85 90 95
Pro Thr Phe Gly Gln Gly Thr Lys Val Glu Ile Lys
100 105

SEQ ID NO: 116
<212> DNA
<223> 3F2 VL
gagatcgtgc tgacccagtc tcccggcacc ctgagcctga gccctggcga gagagccacc 60
ctgagctgca gagccagcca gagcgtgacc agcagctacc tggcctggta tcagcagaag 120
cccggccagg cccccagact gctgatcaac gtgggcagca gacgggccac cggcatcccc 180
gatagattca gcggcagcgg ctccggcacc gacttcaccc tgaccatcag ccggctggaa 240
cccgaggact tcgccgtgta ctactgccag cagggcatca tgctgccccc caccttcggc 300
cagggcacca aggtggaaat caag 324

SEQ ID NO: 117
<212> PRT
<223> CH1A1A HCDR1
Glu Phe Gly Met Asn
1 5

SEQ ID NO: 118
<212> DNA
<223> CH1A1A HCDR1
gagttcggca tgaac 15

SEQ ID NO: 119
<212> PRT
<223> CH1A1A HCDR2
Trp Ile Asn Thr Lys Thr Gly Glu Ala Thr Tyr Val Glu Glu Phe Lys
1 5 10 15
Gly

SEQ ID NO: 120
<212> DNA
<223> CH1A1A HCDR2
tggatcaaca ccaagaccgg cgaggccacc tacgtggaag agttcaaggg c 51

SEQ ID NO: 121
<212> PRT
<223> CH1A1A HCDR3
Trp Asp Phe Ala Tyr Tyr Val Glu Ala Met Asp Tyr
1 5 10

SEQ ID NO: 122
<212> DNA
<223> CH1A1A HCDR3
tgggacttcg cctattacgt ggaagccatg gactac 36

SEQ ID NO: 123
<212> PRT
<223> CH1A1A VH
Gln Val Gln Leu Val Gln Ser Gly Ala Glu Val Lys Lys Pro Gly Ala
1 5 10 15
Ser Val Lys Val Ser Cys Lys Ala Ser Gly Tyr Thr Phe Thr Glu Phe
20 25 30
Gly Met Asn Trp Val Arg Gln Ala Pro Gly Gln Gly Leu Glu Trp Met
35 40 45
Gly Trp Ile Asn Thr Lys Thr Gly Glu Ala Thr Tyr Val Glu Glu Phe
50 55 60
Lys Gly Arg Val Thr Phe Thr Thr Asp Thr Ser Thr Ser Thr Ala Tyr
65 70 75 80
Met Glu Leu Arg Ser Leu Arg Ser Asp Asp Thr Ala Val Tyr Tyr Cys
85 90 95
Ala Arg Trp Asp Phe Ala Tyr Tyr Val Glu Ala Met Asp Tyr Trp Gly
100 105 110
Gln Gly Thr Thr Val Thr Val Ser Ser
115 120

SEQ ID NO: 124
<212> DNA
<223> CH1A1A VH
caggtgcagc tggtgcagtc tggcgccgaa gtgaagaaac ctggagctag tgtgaaggtg 60
tcctgcaagg ccagcggcta caccttcacc gagttcggca tgaactgggt ccgacaggct 120
ccaggccagg gcctcgaatg gatgggctgg atcaacacca agaccggcga ggccacctac 180
gtggaagagt tcaagggcag agtgaccttc accacggaca ccagcaccag caccgcctac 240
atggaactgc ggagcctgag aagcgacgac accgccgtgt actactgcgc cagatgggac 300
ttcgcctatt acgtggaagc catggactac tggggccagg gcaccaccgt gaccgtgtct 360
agc 363

SEQ ID NO: 125
<212> PRT
<223> CH1A1A LCDR1
Lys Ala Ser Ala Ala Val Gly Thr Tyr Val Ala
1 5 10

SEQ ID NO: 126
<212> DNA
<223> CH1A1A LCDR1
aaggccagtg cggctgtggg tacgtatgtt gcg 33

SEQ ID NO: 127
<212> PRT
<223> CH1A1A LCDR2
Ser Ala Ser Tyr Arg Lys Arg
1 5

SEQ ID NO: 128
<212> DNA
<223> CH1A1A LCDR2
tcggcatcct accgcaaaag g 21

SEQ ID NO: 129
<212> PRT
<223> CH1A1A LCDR3
His Gln Tyr Tyr Thr Tyr Pro Leu Phe Thr
1 5 10

SEQ ID NO: 130
<212> DNA
<223> CH1A1A LCDR3
caccaatatt acacctatcc tctattcacg 30

SEQ ID NO: 131
<212> PRT
<223> CH1A1A VL
Asp Ile Gln Met Thr Gln Ser Pro Ser Ser Leu Ser Ala Ser Val Gly
1 5 10 15
Asp Arg Val Thr Ile Thr Cys Lys Ala Ser Ala Ala Val Gly Thr Tyr
20 25 30
Val Ala Trp Tyr Gln Gln Lys Pro Gly Lys Ala Pro Lys Leu Leu Ile
35 40 45
Tyr Ser Ala Ser Tyr Arg Lys Arg Gly Val Pro Ser Arg Phe Ser Gly
50 55 60
Ser Gly Ser Gly Thr Asp Phe Thr Leu Thr Ile Ser Ser Leu Gln Pro
65 70 75 80
Glu Asp Phe Ala Thr Tyr Tyr Cys His Gln Tyr Tyr Thr Tyr Pro Leu
85 90 95
Phe Thr Phe Gly Gln Gly Thr Lys Leu Glu Ile Lys
100 105

SEQ ID NO: 132
<212> DNA
<223> CH1A1A VL
gatatccaga tgacccagtc tccatcctcc ctgtctgcat ctgtgggaga cagagtcacc 60
atcacttgca aggccagtgc ggctgtgggt acgtatgttg cgtggtatca gcagaaacca 120
gggaaagcac ctaagctcct gatctattcg gcatcctacc gcaaaagggg agtcccatca 180
aggttcagtg gcagtggatc tgggacagat ttcactctca ccatcagcag tctgcaacct 240
gaagatttcg caacttacta ctgtcaccaa tattacacct atcctctatt cacgtttggc 300
cagggcacca agctcgagat caag 324

SEQ ID NO: 133
<212> PRT
<223> Anti-CD33 HCDR1
Gly Tyr Thr Ile Thr Asp Ser Asn Ile His
1 5 10

SEQ ID NO: 134
<212> DNA
<223> Anti-CD33 HCDR1
ggctacacca tcaccgacag caacatccac 30

SEQ ID NO: 135
<212> PRT
<223> Anti-CD33 HCDR2
Tyr Ile Tyr Pro Tyr Asn Gly Gly Thr Asp Tyr Asn Gln
1 5 10

SEQ ID NO: 136
<212> DNA
<223> Anti-CD33 HCDR2
tacatctacc cctacaacgg cggcaccgac tacaaccag 39

SEQ ID NO: 137
<212> PRT
<223> Anti-CD33 HCDR3
Gly Asn Pro Trp Leu Ala Tyr
1 5

SEQ ID NO: 138
<212> DNA
<223> Anti-CD33 HCDR3
ggcaacccct ggctggccta t 21

SEQ ID NO: 139
<212> PRT
<223> Anti-CD33 VH
Glu Val Gln Leu Val Gln Ser Gly Ala Glu Val Lys Lys Pro Gly Ser
1 5 10 15
Ser Val Lys Val Ser Cys Lys Ala Ser Gly Tyr Thr Ile Thr Asp Ser
20 25 30
Asn Ile His Trp Val Arg Gln Ala Pro Gly Gln Ser Leu Glu Trp Ile
35 40 45
Gly Tyr Ile Tyr Pro Tyr Asn Gly Gly Thr Asp Tyr Asn Gln Lys Phe
50 55 60
Lys Asn Arg Ala Thr Leu Thr Val Asp Asn Pro Thr Asn Thr Ala Tyr
65 70 75 80
Met Glu Leu Ser Ser Leu Arg Ser Glu Asp Thr Ala Phe Tyr Tyr Cys
85 90 95
Val Asn Gly Asn Pro Trp Leu Ala Tyr Trp Gly Gln Gly Thr Leu Val
100 105 110
Thr Val Ser Ser
115

SEQ ID NO: 140
<212> DNA
<223> Anti-CD33 VH
gaagtgcagc tggtgcagtc tggcgccgaa gtgaagaaac ccggcagcag cgtgaaggtg 60
tcctgcaagg ccagcggcta caccatcacc gacagcaaca tccactgggt ccgacaggcc 120
cctgggcaga gcctggaatg gatcggctac atctacccct acaacggcgg caccgactac 180
aaccagaagt tcaagaaccg ggccaccctg accgtggaca accccaccaa caccgcctac 240
atggaactga gcagcctgcg gagcgaggac accgccttct actactgcgt gaacggcaac 300
ccctggctgg cctattgggg ccagggaacc ctggtcaccg tgtctagc 348

SEQ ID NO: 141
<212> PRT
<223> Anti-CD33 LCDR1
Arg Ala Ser Glu Ser Leu Asp Asn Tyr Gly Ile Arg Phe Leu Thr
1 5 10 15

SEQ ID NO: 142
<212> DNA
<223> Anti-CD33 LCDR1
cgggccagcg agagcctgga caactacggc atccggtttc tgacc 45

SEQ ID NO: 143
<212> PRT
<223> Anti-CD33 LCDR2
Ala Ala Ser Asn Gln Gly Ser
1 5

SEQ ID NO: 144
<212> DNA
<223> Anti-CD33 LCDR2
gccgccagca accagggcag c 21

SEQ ID NO: 145
<212> PRT
<223> Anti-CD33 LCDR3
Gln Gln Thr Lys Glu Val Pro Trp Ser
1 5

SEQ ID NO: 146
<212> DNA
<223> Anti-CD33 LCDR3
cagcagacca aagaggtgcc ctggtcc 27

SEQ ID NO: 147
<212> PRT
<223> Anti-CD33 VL
Asp Ile Gln Leu Thr Gln Ser Pro Ser Thr Leu Ser Ala Ser Val Gly
1 5 10 15
Asp Arg Val Thr Ile Thr Cys Arg Ala Ser Glu Ser Leu Asp Asn Tyr
20 25 30
Gly Ile Arg Phe Leu Thr Trp Phe Gln Gln Lys Pro Gly Lys Ala Pro
35 40 45
Lys Leu Leu Met Tyr Ala Ala Ser Asn Gln Gly Ser Gly Val Pro Ser
50 55 60
Arg Phe Ser Gly Ser Gly Ser Gly Thr Glu Phe Thr Leu Thr Ile Ser
65 70 75 80
Ser Leu Gln Pro Asp Asp Phe Ala Thr Tyr Tyr Cys Gln Gln Thr Lys
85 90 95
Glu Val Pro Trp Ser Phe Gly Gln Gly Thr Lys Val Glu Val Lys
100 105 110

SEQ ID NO: 148
<212> DNA
<223> Anti-CD33 VL
gacatccagc tgacccagag ccccagcacc ctgtctgcca gcgtgggcga cagagtgacc 60
atcacctgtc gggccagcga gagcctggac aactacggca tccggtttct gacctggttc 120
cagcagaagc ccggcaaggc ccccaagctg ctgatgtacg ccgccagcaa ccagggcagc 180
ggcgtgccaa gcagattcag cggcagcggc tccggcaccg agttcaccct gaccatcagc 240
agcctgcagc ccgacgactt cgccacctac tactgccagc agaccaaaga ggtgccctgg 300
tccttcggcc agggcaccaa ggtggaagtg aag 333

SEQ ID NO: 149
<212> PRT
Asp Lys Thr His Thr Cys Pro Pro Cys Pro Ala Pro Glu Leu Leu Gly
1 5 10 15
Gly Pro Ser Val Phe Leu Phe Pro Pro Lys Pro Lys Asp Thr Leu Met
20 25 30
Ile Ser Arg Thr Pro Glu Val Thr Cys Val Val Val Asp Val Ser His
35 40 45
Glu Asp Pro Glu Val Lys Phe Asn Trp Tyr Val Asp Gly Val Glu Val
50 55 60
His Asn Ala Lys Thr Lys Pro Arg Glu Glu Gln Tyr Asn Ser Thr Tyr
65 70 75 80
Arg Val Val Ser Val Leu Thr Val Leu His Gln Asp Trp Leu Asn Gly
85 90 95
Lys Glu Tyr Lys Cys Lys Val Ser Asn Lys Ala Leu Pro Ala Pro Ile
100 105 110
Glu Lys Thr Ile Ser Lys Ala Lys Gly Gln Pro Arg Glu Pro Gln Val
115 120 125
Tyr Thr Leu Pro Pro Ser Arg Asp Glu Leu Thr Lys Asn Gln Val Ser
130 135 140
Leu Thr Cys Leu Val Lys Gly Phe Tyr Pro Ser Asp Ile Ala Val Glu
145 150 155 160
Trp Glu Ser Asn Gly Gln Pro Glu Asn Asn Tyr Lys Thr Thr Pro Pro
165 170 175
Val Leu Asp Ser Asp Gly Ser Phe Phe Leu Tyr Ser Lys Leu Thr Val
180 185 190
Asp Lys Ser Arg Trp Gln Gln Gly Asn Val Phe Ser Cys Ser Val Met
195 200 205
His Glu Ala Leu His Asn His Tyr Thr Gln Lys Ser Leu Ser Leu Ser
210 215 220
Pro Gly Lys
225

SEQ ID NO: 150
<212> PRT
<223> Linker
Glu Pro Lys Ser Cys Gly Gly Gly Gly Ser Gly Gly Gly Gly Ser
1 5 10 15

SEQ ID NO: 151
<212> PRT
<223> Linker
Glu Pro Lys Ser Cys Asp Gly Gly Gly Gly Ser Gly Gly Gly Gly Ser
1 5 10 15

SEQ ID NO: 152
<212> PRT
<223> Linker
Gly Gly Gly Gly Ser Gly Gly Gly Gly Ser Gly Gly Gly Gly Ser Gly
1 5 10 15
Gly Gly Gly Ser Gly Gly Gly Gly Ser Gly Gly Gly Gly Ser Gly Gly
20 25 30

SEQ ID NO: 153
<212> PRT
<223> Linker
Ser Gly Gly Gly Ser Gly Gly Gly Ser Glu Gly Gly Gly Ser Glu Gly
1 5 10 15
Gly Gly Ser Glu Gly Gly Gly Ser Glu Gly Gly Gly Ser Gly Gly Gly
20 25 30
Ser Gly

SEQ ID NO: 154
<212> PRT
<223> Leader 1
Met Asp Trp Thr Trp Arg Ile Leu Phe Leu Val Ala Ala Ala Thr Gly
1 5 10 15
Ala His Ser

SEQ ID NO: 155
<212> DNA
<223> Leader 1
atggactgga cctggagaat cctcttcttg gtggcagcag ccacaggagc ccactcc 57

SEQ ID NO: 156
<212> DNA
<223> Leader 1
atggactgga cctggaggat cctcttcttg gtggcagcag ccacaggagc ccactcc 57

SEQ ID NO: 157
<212> PRT
<223> Leader 2
Met Asp Met Arg Val Pro Ala Gln Leu Leu Gly Leu Leu Leu Leu Trp
1 5 10 15
Phe Pro Gly Ala Arg Cys
20

SEQ ID NO: 158
<212> DNA
<223> Leader 2
atggacatga gggtccccgc tcagctcctg ggcctcctgc tgctctggtt cccaggtgcc 60
aggtgt 66

SEQ ID NO: 159
<212> PRT
<223> Leader 3
Met Gly Trp Ser Cys Ile Ile Leu Phe Leu Val Ala Thr Ala Thr Gly
1 5 10 15
Val His Ser

SEQ ID NO: 160
<212> DNA
<223> Leader 3
atgggatgga gctgtatcat cctcttcttg gtagcaacag ctaccggtgt gcattcc 57

SEQ ID NO: 161
<212> DNA
<223> Leader 3
atgggctggt cctgcatcat cctgtttctg gtggctaccg ccactggagt gcattcc 57

SEQ ID NO: 162
<212> DNA
<223> Leader 3
atgggctggt cctgcatcat cctgtttctg gtcgccacag ccaccggcgt gcactct 57

SEQ ID NO: 163
<212> PRT
<223> V9 HCDR1
Gly Tyr Thr Met Asn
1 5

SEQ ID NO: 164
<212> DNA
<223> V9 HCDR1
ggctacacca tgaac 15

SEQ ID NO: 165
<212> PRT
<223> V9 HCDR2
Leu Ile Asn Pro Tyr Lys Gly Val Ser Thr Tyr Asn Gln Lys Phe Lys
1 5 10 15
Asp

SEQ ID NO: 166
<212> DNA
<223> V9 HCDR2
ctgatcaacc cctacaaggg cgtgagcacc tacaaccaga agttcaagga c 51

SEQ ID NO: 167
<212> PRT
<223> V9 HCDR3
Ser Gly Tyr Tyr Gly Asp Ser Asp Trp Tyr Phe Asp Val
1 5 10

SEQ ID NO: 168
<212> DNA
<223> V9 HCDR3
agcggctact acggcgacag cgactggtac ttcgacgtg 39

SEQ ID NO: 169
<212> PRT
<223> V9 VH
Glu Val Gln Leu Val Glu Ser Gly Gly Gly Leu Val Gln Pro Gly Gly
1 5 10 15
Ser Leu Arg Leu Ser Cys Ala Ala Ser Gly Tyr Ser Phe Thr Gly Tyr
20 25 30
Thr Met Asn Trp Val Arg Gln Ala Pro Gly Lys Gly Leu Glu Trp Val
35 40 45
Ala Leu Ile Asn Pro Tyr Lys Gly Val Ser Thr Tyr Asn Gln Lys Phe
50 55 60
Lys Asp Arg Phe Thr Ile Ser Val Asp Lys Ser Lys Asn Thr Ala Tyr
65 70 75 80
Leu Gln Met Asn Ser Leu Arg Ala Glu Asp Thr Ala Val Tyr Tyr Cys
85 90 95
Ala Arg Ser Gly Tyr Tyr Gly Asp Ser Asp Trp Tyr Phe Asp Val Trp
100 105 110
Gly Gln Gly Thr Leu Val Thr Val Ser Ser
115 120

SEQ ID NO: 170
<212> DNA
<223> V9 VH
gaggtgcagc tggtcgagtc cggcggaggc ctggtgcagc ctggcggcag cctgagactg 60
agctgcgccg ccagcggcta cagcttcacc ggctacacca tgaactgggt ccggcaggct 120
cctggcaagg gcctcgaatg ggtggccctg atcaacccct acaagggcgt gagcacctac 180
aaccagaagt tcaaggaccg gttcaccatc agcgtggaca agagcaagaa caccgcctat 240
ctgcagatga acagcctgcg ggccgaggac accgccgtgt actactgcgc cagaagcggc 300
tactacggcg acagcgactg gtacttcgac gtgtggggcc agggcacact ggtcaccgtg 360
tccagc 366

SEQ ID NO: 171
<212> PRT
<223> V9 LCDR1
Arg Ala Ser Gln Asp Ile Arg Asn Tyr Leu Asn
1 5 10

SEQ ID NO: 172
<212> DNA
<223> V9 LCDR1
cgggccagcc aggacatcag aaactacctg aac 33

SEQ ID NO: 173
<212> PRT
<223> V9 LCDR2
Tyr Thr Ser Arg Leu Glu Ser
1 5

SEQ ID NO: 174
<212> DNA
<223> V9 LCDR2
tacacctcta gactggaaag c 21

SEQ ID NO: 175
<212> PRT
<223> V9 LCDR3
Gln Gln Gly Asn Thr Leu Pro Trp Thr
1 5

SEQ ID NO: 176
<212> DNA
<223> V9 LCDR3
cagcagggca acacactccc ctggacc 27

SEQ ID NO: 177
<212> PRT
<223> V9 VL
Asp Ile Gln Met Thr Gln Ser Pro Ser Ser Leu Ser Ala Ser Val Gly
1 5 10 15
Asp Arg Val Thr Ile Thr Cys Arg Ala Ser Gln Asp Ile Arg Asn Tyr
20 25 30
Leu Asn Trp Tyr Gln Gln Lys Pro Gly Lys Ala Pro Lys Leu Leu Ile
35 40 45
Tyr Tyr Thr Ser Arg Leu Glu Ser Gly Val Pro Ser Arg Phe Ser Gly
50 55 60
Ser Gly Ser Gly Thr Asp Tyr Thr Leu Thr Ile Ser Ser Leu Gln Pro
65 70 75 80
Glu Asp Phe Ala Thr Tyr Tyr Cys Gln Gln Gly Asn Thr Leu Pro Trp
85 90 95
Thr Phe Gly Gln Gly Thr Lys Val Glu Ile Lys
100 105

SEQ ID NO: 178
<212> DNA
<223> V9 VL
gacatccaga tgacccagag cccctctagc ctgagcgcca gcgtgggcga cagagtgacc 60
atcacctgtc gggccagcca ggacatcaga aactacctga actggtatca gcagaagccc 120
ggcaaggccc ccaagctgct gatctactac acctctagac tggaaagcgg cgtgcccagc 180
cggtttagcg gcagcggctc cggcaccgac tacaccctga ccatcagcag cctgcagccc 240
gaggacttcg ccacctacta ctgccagcag ggcaacacac tcccctggac cttcggccag 300
ggcaccaagg tggagatcaa g 321

SEQ ID NO: 179
<212> PRT
<223> V9(VH-CL)-LC007(VL-CL)
Glu Val Gln Leu Val Glu Ser Gly Gly Gly Leu Val Gln Pro Gly Gly
1 5 10 15
Ser Leu Arg Leu Ser Cys Ala Ala Ser Gly Tyr Ser Phe Thr Gly Tyr
20 25 30
Thr Met Asn Trp Val Arg Gln Ala Pro Gly Lys Gly Leu Glu Trp Val
35 40 45
Ala Leu Ile Asn Pro Tyr Lys Gly Val Ser Thr Tyr Asn Gln Lys Phe
50 55 60
Lys Asp Arg Phe Thr Ile Ser Val Asp Lys Ser Lys Asn Thr Ala Tyr
65 70 75 80
Leu Gln Met Asn Ser Leu Arg Ala Glu Asp Thr Ala Val Tyr Tyr Cys
85 90 95
Ala Arg Ser Gly Tyr Tyr Gly Asp Ser Asp Trp Tyr Phe Asp Val Trp
100 105 110
Gly Gln Gly Thr Leu Val Thr Val Ser Ser Ala Ser Val Ala Ala Pro
115 120 125
Ser Val Phe Ile Phe Pro Pro Ser Asp Glu Gln Leu Lys Ser Gly Thr
130 135 140
Ala Ser Val Val Cys Leu Leu Asn Asn Phe Tyr Pro Arg Glu Ala Lys
145 150 155 160
Val Gln Trp Lys Val Asp Asn Ala Leu Gln Ser Gly Asn Ser Gln Glu
165 170 175
Ser Val Thr Glu Gln Asp Ser Lys Asp Ser Thr Tyr Ser Leu Ser Ser
180 185 190
Thr Leu Thr Leu Ser Lys Ala Asp Tyr Glu Lys His Lys Val Tyr Ala
195 200 205
Cys Glu Val Thr His Gln Gly Leu Ser Ser Pro Val Thr Lys Ser Phe
210 215 220
Asn Arg Gly Glu Cys Asp Gly Gly Gly Gly Ser Gly Gly Gly Gly Ser
225 230 235 240
Asp Ile Val Leu Thr Gln Ser Pro Ser Ser Leu Ser Ala Ser Leu Gly
245 250 255
Asp Arg Val Thr Ile Ser Cys Ser Ala Ser Gln Gly Ile Arg Asn Tyr
260 265 270
Leu Asn Trp Tyr Gln Gln Arg Pro Asp Gly Thr Val Lys Leu Leu Ile
275 280 285
Tyr Tyr Thr Ser Ser Leu His Ser Gly Val Pro Ser Arg Phe Ser Gly
290 295 300
Ser Gly Ser Gly Thr Asp Tyr Ser Leu Thr Ile Ser Asn Leu Glu Pro
305 310 315 320
Glu Asp Ile Ala Thr Tyr Tyr Cys Gln Gln Tyr Ser Lys Leu Pro Trp
325 330 335
Thr Phe Gly Gly Gly Thr Lys Leu Glu Ile Lys Arg Thr Val Ala Ala
340 345 350
Pro Ser Val Phe Ile Phe Pro Pro Ser Asp Glu Gln Leu Lys Ser Gly
355 360 365
Thr Ala Ser Val Val Cys Leu Leu Asn Asn Phe Tyr Pro Arg Glu Ala
370 375 380
Lys Val Gln Trp Lys Val Asp Asn Ala Leu Gln Ser Gly Asn Ser Gln
385 390 395 400
Glu Ser Val Thr Glu Gln Asp Ser Lys Asp Ser Thr Tyr Ser Leu Ser
405 410 415
Ser Thr Leu Thr Leu Ser Lys Ala Asp Tyr Glu Lys His Lys Val Tyr
420 425 430
Ala Cys Glu Val Thr His Gln Gly Leu Ser Ser Pro Val Thr Lys Ser
435 440 445
Phe Asn Arg Gly Glu Cys
450

SEQ ID NO: 180
<212> DNA
<223> V9(VH-CL)-LC007(VL-CL)
gaggtgcagc tggtggaatc tggcggcgga ctggtgcagc ctggcggatc tctgagactg 60
agctgtgccg ccagcggcta cagcttcacc ggctacacca tgaactgggt gcgccaggcc 120
cctggcaagg gactggaatg ggtggccctg atcaacccct acaagggcgt gtccacctac 180
aaccagaagt tcaaggaccg gttcaccatc agcgtggaca agagcaagaa caccgcctac 240
ctgcagatga acagcctgcg ggccgaggac accgccgtgt actattgtgc cagaagcggc 300
tactacggcg acagcgactg gtacttcgac gtgtggggcc agggcacact cgtgaccgtg 360
tcaagcgcta gcgtggccgc tcccagcgtg ttcatcttcc cacctagcga cgagcagctg 420
aagtccggca cagcctctgt cgtgtgcctg ctgaacaact tctacccccg cgaggccaag 480
gtgcagtgga aggtggacaa tgccctgcag agcggcaaca gccaggaaag cgtgaccgag 540
caggacagca aggatagcac ctacagcctg agcagcaccc tgaccctgag caaggccgac 600
tacgagaagc acaaggtgta cgcctgcgaa gtgacccacc agggcctgtc tagccccgtg 660
accaagagct tcaaccgggg cgagtgtgat ggcggaggcg gatccggggg aggcggctct 720
gatattgtgc tgacccagag ccccagcagc ctgtctgcct ctctgggcga cagagtgacc 780
atcagctgta gcgcctctca gggcatccgg aactacctga actggtatca gcagcggccc 840
gacggcaccg tgaagctgct gatctactac accagctccc tgcactccgg cgtgcccagc 900
agattttctg gcagcggctc cggcaccgac tactccctga ccatctccaa cctggaaccc 960
gaggatatcg ccacctacta ctgccagcag tactccaagc tgccctggac ctttggaggc 1020
ggcaccaagc tggaaatcaa gcgtacggtg gctgccccct ccgtgtttat ctttccccca 1080
tccgatgaac agctgaaaag cggcaccgcc agcgtcgtgt gtctgctgaa caatttttac 1140
cctagggaag ctaaagtgca gtggaaagtg gataacgcac tgcagtccgg caactcccag 1200
gaatctgtga cagaacagga ctctaaggac agcacatact ccctgtcctc caccctgaca 1260
ctgtctaagg ctgattatga gaaacacaaa gtgtatgctt gtgaagtgac acatcaggga 1320
ctgagcagcc ctgtgacaaa gtccttcaac agaggcgagt gc 1362

SEQ ID NO: 181
<212> PRT
<223> Fc(knob) P329G LALA
Asp Lys Thr His Thr Cys Pro Pro Cys Pro Ala Pro Glu Ala Ala Gly
1 5 10 15
Gly Pro Ser Val Phe Leu Phe Pro Pro Lys Pro Lys Asp Thr Leu Met
20 25 30
Ile Ser Arg Thr Pro Glu Val Thr Cys Val Val Val Asp Val Ser His
35 40 45
Glu Asp Pro Glu Val Lys Phe Asn Trp Tyr Val Asp Gly Val Glu Val
50 55 60
His Asn Ala Lys Thr Lys Pro Arg Glu Glu Gln Tyr Asn Ser Thr Tyr
65 70 75 80
Arg Val Val Ser Val Leu Thr Val Leu His Gln Asp Trp Leu Asn Gly
85 90 95
Lys Glu Tyr Lys Cys Lys Val Ser Asn Lys Ala Leu Gly Ala Pro Ile
100 105 110
Glu Lys Thr Ile Ser Lys Ala Lys Gly Gln Pro Arg Glu Pro Gln Val
115 120 125
Tyr Thr Leu Pro Pro Cys Arg Asp Glu Leu Thr Lys Asn Gln Val Ser
130 135 140
Leu Trp Cys Leu Val Lys Gly Phe Tyr Pro Ser Asp Ile Ala Val Glu
145 150 155 160
Trp Glu Ser Asn Gly Gln Pro Glu Asn Asn Tyr Lys Thr Thr Pro Pro
165 170 175
Val Leu Asp Ser Asp Gly Ser Phe Phe Leu Tyr Ser Lys Leu Thr Val
180 185 190
Asp Lys Ser Arg Trp Gln Gln Gly Asn Val Phe Ser Cys Ser Val Met
195 200 205
His Glu Ala Leu His Asn His Tyr Thr Gln Lys Ser Leu Ser Leu Ser
210 215 220
Pro Gly Lys
225

SEQ ID NO: 182
<212> DNA
<223> Fc(knob) P329G LALA
gacaaaactc acacatgccc accgtgccca gcacctgaag ctgcaggggg accgtcagtc 60
ttcctcttcc ccccaaaacc caaggacacc ctcatgatct cccggacccc tgaggtcaca 120
tgcgtggtgg tggacgtgag ccacgaagac cctgaggtca agttcaactg gtacgtggac 180
ggcgtggagg tgcataatgc caagacaaag ccgcgggagg agcagtacaa cagcacgtac 240
cgtgtggtca gcgtcctcac cgtcctgcac caggactggc tgaatggcaa ggagtacaag 300
tgcaaggtct ccaacaaagc cctcggcgcc cccatcgaga aaaccatctc caaagccaaa 360
gggcagcccc gagaaccaca ggtgtacacc ctgcccccat gccgggatga gctgaccaag 420
aaccaggtca gcctgtggtg cctggtcaaa ggcttctatc ccagcgacat cgccgtggag 480
tgggagagca atgggcagcc ggagaacaac tacaagacca cgcctcccgt gctggactcc 540
gacggctcct tcttcctcta cagcaagctc accgtggaca agagcaggtg gcagcagggg 600
aacgtcttct catgctccgt gatgcatgag gctctgcaca accactacac gcagaagagc 660
ctctccctgt ctccgggtaa a 681

SEQ ID NO: 183
<212> PRT
<223> V9(VL-CH1)
Asp Ile Gln Met Thr Gln Ser Pro Ser Ser Leu Ser Ala Ser Val Gly
1 5 10 15
Asp Arg Val Thr Ile Thr Cys Arg Ala Ser Gln Asp Ile Arg Asn Tyr
20 25 30
Leu Asn Trp Tyr Gln Gln Lys Pro Gly Lys Ala Pro Lys Leu Leu Ile
35 40 45
Tyr Tyr Thr Ser Arg Leu Glu Ser Gly Val Pro Ser Arg Phe Ser Gly
50 55 60
Ser Gly Ser Gly Thr Asp Tyr Thr Leu Thr Ile Ser Ser Leu Gln Pro
65 70 75 80
Glu Asp Phe Ala Thr Tyr Tyr Cys Gln Gln Gly Asn Thr Leu Pro Trp
85 90 95
Thr Phe Gly Gln Gly Thr Lys Val Glu Ile Lys Ser Ser Ala Ser Thr
100 105 110
Lys Gly Pro Ser Val Phe Pro Leu Ala Pro Ser Ser Lys Ser Thr Ser
115 120 125
Gly Gly Thr Ala Ala Leu Gly Cys Leu Val Lys Asp Tyr Phe Pro Glu
130 135 140
Pro Val Thr Val Ser Trp Asn Ser Gly Ala Leu Thr Ser Gly Val His
145 150 155 160
Thr Phe Pro Ala Val Leu Gln Ser Ser Gly Leu Tyr Ser Leu Ser Ser
165 170 175
Val Val Thr Val Pro Ser Ser Ser Leu Gly Thr Gln Thr Tyr Ile Cys
180 185 190
Asn Val Asn His Lys Pro Ser Asn Thr Lys Val Asp Lys Lys Val Glu
195 200 205
Pro Lys Ser Cys
210

SEQ ID NO: 184
<212> DNA
<223> V9(VL-CH1)
gatattcaga tgacccagag ccccagctct ctgagcgcca gcgtgggcga cagagtgacc 60
atcacctgtc gggccagcca ggacatcaga aactacctga actggtatca gcagaagccc 120
ggcaaggccc ccaagctgct gatctactac accagcagac tggaaagcgg cgtgccctcc 180
agattttccg gcagcggctc cggcaccgac tacaccctga ccatcagcag cctgcagccc 240
gaggatttcg ccacatatta ctgccagcag ggcaataccc tgccctggac cttcggacag 300
ggcacaaaag tggaaatcaa gagcagcgct tccaccaaag gcccttccgt gtttcctctg 360
gctcctagct ccaagtccac ctctggaggc accgctgctc tcggatgcct cgtgaaggat 420
tattttcctg agcctgtgac agtgtcctgg aatagcggag cactgacctc tggagtgcat 480
actttccccg ctgtgctgca gtcctctgga ctgtacagcc tgagcagcgt ggtgacagtg 540
cccagcagca gcctgggcac ccagacctac atctgcaacg tgaaccacaa gcccagcaac 600
accaaggtgg acaagaaggt ggaacccaag tcttgt 636

SEQ ID NO: 185
<212> PRT
<223> V9(VH-CL)-Fc(knob) P329G LALA
Glu Val Gln Leu Val Glu Ser Gly Gly Gly Leu Val Gln Pro Gly Gly
1 5 10 15
Ser Leu Arg Leu Ser Cys Ala Ala Ser Gly Tyr Ser Phe Thr Gly Tyr
20 25 30
Thr Met Asn Trp Val Arg Gln Ala Pro Gly Lys Gly Leu Glu Trp Val
35 40 45
Ala Leu Ile Asn Pro Tyr Lys Gly Val Ser Thr Tyr Asn Gln Lys Phe
50 55 60
Lys Asp Arg Phe Thr Ile Ser Val Asp Lys Ser Lys Asn Thr Ala Tyr
65 70 75 80
Leu Gln Met Asn Ser Leu Arg Ala Glu Asp Thr Ala Val Tyr Tyr Cys
85 90 95
Ala Arg Ser Gly Tyr Tyr Gly Asp Ser Asp Trp Tyr Phe Asp Val Trp
100 105 110
Gly Gln Gly Thr Leu Val Thr Val Ser Ser Ala Ser Val Ala Ala Pro
115 120 125
Ser Val Phe Ile Phe Pro Pro Ser Asp Glu Gln Leu Lys Ser Gly Thr
130 135 140
Ala Ser Val Val Cys Leu Leu Asn Asn Phe Tyr Pro Arg Glu Ala Lys
145 150 155 160
Val Gln Trp Lys Val Asp Asn Ala Leu Gln Ser Gly Asn Ser Gln Glu
165 170 175
Ser Val Thr Glu Gln Asp Ser Lys Asp Ser Thr Tyr Ser Leu Ser Ser
180 185 190
Thr Leu Thr Leu Ser Lys Ala Asp Tyr Glu Lys His Lys Val Tyr Ala
195 200 205
Cys Glu Val Thr His Gln Gly Leu Ser Ser Pro Val Thr Lys Ser Phe
210 215 220
Asn Arg Gly Glu Cys Asp Lys Thr His Thr Cys Pro Pro Cys Pro Ala
225 230 235 240
Pro Glu Ala Ala Gly Gly Pro Ser Val Phe Leu Phe Pro Pro Lys Pro
245 250 255
Lys Asp Thr Leu Met Ile Ser Arg Thr Pro Glu Val Thr Cys Val Val
260 265 270
Val Asp Val Ser His Glu Asp Pro Glu Val Lys Phe Asn Trp Tyr Val
275 280 285
Asp Gly Val Glu Val His Asn Ala Lys Thr Lys Pro Arg Glu Glu Gln
290 295 300
Tyr Asn Ser Thr Tyr Arg Val Val Ser Val Leu Thr Val Leu His Gln
305 310 315 320
Asp Trp Leu Asn Gly Lys Glu Tyr Lys Cys Lys Val Ser Asn Lys Ala
325 330 335
Leu Gly Ala Pro Ile Glu Lys Thr Ile Ser Lys Ala Lys Gly Gln Pro
340 345 350
Arg Glu Pro Gln Val Tyr Thr Leu Pro Pro Cys Arg Asp Glu Leu Thr
355 360 365
Lys Asn Gln Val Ser Leu Trp Cys Leu Val Lys Gly Phe Tyr Pro Ser
370 375 380
Asp Ile Ala Val Glu Trp Glu Ser Asn Gly Gln Pro Glu Asn Asn Tyr
385 390 395 400
Lys Thr Thr Pro Pro Val Leu Asp Ser Asp Gly Ser Phe Phe Leu Tyr
405 410 415
Ser Lys Leu Thr Val Asp Lys Ser Arg Trp Gln Gln Gly Asn Val Phe
420 425 430
Ser Cys Ser Val Met His Glu Ala Leu His Asn His Tyr Thr Gln Lys
435 440 445
Ser Leu Ser Leu Ser Pro Gly Lys
450 455

SEQ ID NO: 186
<212> DNA
<223> V9(VH-CL)-Fc(knob) P329G LALA
gaggtgcagc tggtcgagag cggaggcggc ctggtgcagc ctggcggcag cctgagactg 60
agctgcgccg ccagcggcta cagcttcacc ggctacacca tgaactgggt ccggcaggca 120
cctggcaagg gactggaatg ggtggccctg atcaacccct acaagggcgt gagcacctac 180
aaccagaagt tcaaggaccg gttcaccatc agcgtggaca agagcaagaa caccgcctat 240
ctgcagatga acagcctgcg ggccgaggac accgccgtgt actactgcgc cagaagcggc 300
tactacggcg acagcgactg gtacttcgac gtgtggggcc agggcaccct cgtgaccgtg 360
tctagcgcta gcgtggccgc tccctccgtg tttatctttc ccccatccga tgaacagctg 420
aaaagcggca ccgcctccgt cgtgtgtctg ctgaacaatt tttaccctag ggaagctaaa 480
gtgcagtgga aagtggataa cgcactgcag tccggcaact cccaggaatc tgtgacagaa 540
caggactcca aggacagcac ctactccctg tcctccaccc tgacactgtc taaggctgat 600
tatgagaaac acaaagtcta cgcctgcgaa gtcacccatc agggcctgag ctcgcccgtc 660
acaaagagct tcaacagggg agagtgtgac aagacccaca cctgtccccc ttgtcctgcc 720
cctgaagctg ctggcggccc ttctgtgttc ctgttccccc caaagcccaa ggacaccctg 780
atgatcagcc ggacccccga agtgacctgc gtggtggtgg atgtgtccca cgaggaccct 840
gaagtgaagt tcaattggta cgtggacggc gtggaagtgc acaacgccaa gacaaagccg 900
cgggaggagc agtacaacag cacgtaccgt gtggtcagcg tcctcaccgt cctgcaccag 960
gactggctga atggcaagga gtacaagtgc aaggtctcca acaaagccct cggcgccccc 1020
atcgagaaaa ccatctccaa agccaaaggg cagccccgag aaccacaggt gtacaccctg 1080
cccccatgcc gggatgagct gaccaagaac caggtcagcc tgtggtgcct ggtcaaaggc 1140
ttctatccca gcgacatcgc cgtggagtgg gagagcaatg ggcagccgga gaacaactac 1200
aagaccacgc ctcccgtgct ggactccgac ggctccttct tcctctacag caagctcacc 1260
gtggacaaga gcaggtggca gcaggggaac gtcttctcat gctccgtgat gcatgaggct 1320
ctgcacaacc actacacgca gaagagcctc tccctgtctc cgggtaaa 1368

SEQ ID NO: 187
<212> PRT
<223> LC007(VH-CH1)-V9(VH-CL)-Fc(knob) P329G LALA
Glu Val Gln Leu Gln Glu Ser Gly Pro Gly Leu Val Lys Pro Ser Gln
1 5 10 15
Ser Leu Ser Leu Thr Cys Ser Val Thr Gly Tyr Ser Ile Thr Ser Gly
20 25 30
Tyr Tyr Trp Asn Trp Ile Arg Gln Phe Pro Gly Asn Lys Leu Glu Trp
35 40 45
Met Gly Tyr Ile Thr Tyr Asp Gly Ser Asn Asn Tyr Asn Pro Ser Leu
50 55 60
Lys Asn Arg Ile Ser Ile Thr Arg Asp Thr Ser Lys Asn Gln Phe Phe
65 70 75 80
Leu Lys Leu Asn Ser Val Thr Thr Glu Asp Thr Ala Thr Tyr Tyr Cys
85 90 95
Ala Asp Phe Asp Tyr Trp Gly Gln Gly Thr Thr Leu Thr Val Ser Ser
100 105 110
Ala Ser Thr Lys Gly Pro Ser Val Phe Pro Leu Ala Pro Ser Ser Lys
115 120 125
Ser Thr Ser Gly Gly Thr Ala Ala Leu Gly Cys Leu Val Lys Asp Tyr
130 135 140
Phe Pro Glu Pro Val Thr Val Ser Trp Asn Ser Gly Ala Leu Thr Ser
145 150 155 160
Gly Val His Thr Phe Pro Ala Val Leu Gln Ser Ser Gly Leu Tyr Ser
165 170 175
Leu Ser Ser Val Val Thr Val Pro Ser Ser Ser Leu Gly Thr Gln Thr
180 185 190
Tyr Ile Cys Asn Val Asn His Lys Pro Ser Asn Thr Lys Val Asp Lys
195 200 205
Lys Val Glu Pro Lys Ser Cys Asp Gly Gly Gly Gly Ser Gly Gly Gly
210 215 220
Gly Ser Glu Val Gln Leu Val Glu Ser Gly Gly Gly Leu Val Gln Pro
225 230 235 240
Gly Gly Ser Leu Arg Leu Ser Cys Ala Ala Ser Gly Tyr Ser Phe Thr
245 250 255
Gly Tyr Thr Met Asn Trp Val Arg Gln Ala Pro Gly Lys Gly Leu Glu
260 265 270
Trp Val Ala Leu Ile Asn Pro Tyr Lys Gly Val Ser Thr Tyr Asn Gln
275 280 285
Lys Phe Lys Asp Arg Phe Thr Ile Ser Val Asp Lys Ser Lys Asn Thr
290 295 300
Ala Tyr Leu Gln Met Asn Ser Leu Arg Ala Glu Asp Thr Ala Val Tyr
305 310 315 320
Tyr Cys Ala Arg Ser Gly Tyr Tyr Gly Asp Ser Asp Trp Tyr Phe Asp
325 330 335
Val Trp Gly Gln Gly Thr Leu Val Thr Val Ser Ser Ala Ser Val Ala
340 345 350
Ala Pro Ser Val Phe Ile Phe Pro Pro Ser Asp Glu Gln Leu Lys Ser
355 360 365
Gly Thr Ala Ser Val Val Cys Leu Leu Asn Asn Phe Tyr Pro Arg Glu
370 375 380
Ala Lys Val Gln Trp Lys Val Asp Asn Ala Leu Gln Ser Gly Asn Ser
385 390 395 400
Gln Glu Ser Val Thr Glu Gln Asp Ser Lys Asp Ser Thr Tyr Ser Leu
405 410 415
Ser Ser Thr Leu Thr Leu Ser Lys Ala Asp Tyr Glu Lys His Lys Val
420 425 430
Tyr Ala Cys Glu Val Thr His Gln Gly Leu Ser Ser Pro Val Thr Lys
435 440 445
Ser Phe Asn Arg Gly Glu Cys Asp Lys Thr His Thr Cys Pro Pro Cys
450 455 460
Pro Ala Pro Glu Ala Ala Gly Gly Pro Ser Val Phe Leu Phe Pro Pro
465 470 475 480
Lys Pro Lys Asp Thr Leu Met Ile Ser Arg Thr Pro Glu Val Thr Cys
485 490 495
Val Val Val Asp Val Ser His Glu Asp Pro Glu Val Lys Phe Asn Trp
500 505 510
Tyr Val Asp Gly Val Glu Val His Asn Ala Lys Thr Lys Pro Arg Glu
515 520 525
Glu Gln Tyr Asn Ser Thr Tyr Arg Val Val Ser Val Leu Thr Val Leu
530 535 540
His Gln Asp Trp Leu Asn Gly Lys Glu Tyr Lys Cys Lys Val Ser Asn
545 550 555 560
Lys Ala Leu Gly Ala Pro Ile Glu Lys Thr Ile Ser Lys Ala Lys Gly
565 570 575
Gln Pro Arg Glu Pro Gln Val Tyr Thr Leu Pro Pro Cys Arg Asp Glu
580 585 590
Leu Thr Lys Asn Gln Val Ser Leu Trp Cys Leu Val Lys Gly Phe Tyr
595 600 605
Pro Ser Asp Ile Ala Val Glu Trp Glu Ser Asn Gly Gln Pro Glu Asn
610 615 620
Asn Tyr Lys Thr Thr Pro Pro Val Leu Asp Ser Asp Gly Ser Phe Phe
625 630 635 640
Leu Tyr Ser Lys Leu Thr Val Asp Lys Ser Arg Trp Gln Gln Gly Asn
645 650 655
Val Phe Ser Cys Ser Val Met His Glu Ala Leu His Asn His Tyr Thr
660 665 670
Gln Lys Ser Leu Ser Leu Ser Pro Gly Lys
675 680

SEQ ID NO: 188
<212> DNA
<223> LC007(VH-CH1)- V9(VH-CL)-Fc(knob) P329G LALA
gaggtgcagc tgcaggaatc tggccctggc ctggtcaagc caagccagag tctgagcctg 60
acctgcagcg tgaccggcta cagcattacc agcggctact actggaactg gattcggcag 120
ttccccggca ataagctgga atggatgggc tacatcacct acgacggcag caacaactac 180
aaccccagcc tgaagaaccg gatcagcatc acccgggaca ccagcaagaa ccagttcttc 240
ctgaagctga acagcgtgac caccgaggac accgccacat actattgcgc cgacttcgac 300
tactggggcc agggcaccac cctgaccgtg tccagcgcca gcacaaaggg ccctagcgtg 360
ttccctctgg cccccagcag caagagcaca agcggcggaa cagccgccct gggctgcctc 420
gtgaaggact acttccccga gcccgtgaca gtgtcttgga acagcggagc cctgacaagc 480
ggcgtgcaca ccttccctgc cgtgctgcag agcagcggcc tgtactccct gagcagcgtg 540
gtcaccgtgc ctagcagcag cctgggcacc cagacctaca tctgcaacgt gaaccacaag 600
cccagcaaca ccaaagtgga caagaaggtg gagcccaaga gctgtgatgg cggaggaggg 660
tccggaggcg gaggatccga agtgcagctg gtggaatctg gcggaggcct ggtgcagcct 720
ggcggatctc tgagactgag ctgtgccgcc agcggctaca gcttcaccgg ctacaccatg 780
aactgggtgc gccaggcccc tggcaaggga ctggaatggg tggccctgat caacccctac 840
aagggcgtgt ccacatacaa ccagaagttc aaggaccggt tcaccatcag cgtggacaag 900
agcaagaaca ccgcctacct gcagatgaac agcctgcggg ccgaggacac cgccgtgtac 960
tattgtgcca gaagcggcta ctacggcgac agcgactggt acttcgacgt gtggggccag 1020
ggcacactcg tgaccgtgtc aagcgctagc gtggccgctc cctccgtgtt tatctttccc 1080
ccatccgatg aacagctgaa aagcggcacc gcctccgtcg tgtgtctgct gaacaatttt 1140
taccctaggg aagctaaagt gcagtggaaa gtggataacg cactgcagtc cggcaactcc 1200
caggaatctg tgacagaaca ggactccaag gacagcacct actccctgtc ctccaccctg 1260
acactgtcta aggctgatta tgagaaacac aaagtctacg cctgcgaagt cacccatcag 1320
ggcctgagct cgcccgtcac aaagagcttc aacaggggag agtgtgacaa gacccacacc 1380
tgtccccctt gtcctgcccc tgaagctgct ggcggccctt ctgtgttcct gttcccccca 1440
aagcccaagg acaccctgat gatcagccgg acccccgaag tgacctgcgt ggtggtggat 1500
gtgtcccacg aggaccctga agtgaagttc aattggtacg tggacggcgt ggaagtgcac 1560
aacgccaaga caaagccgcg ggaggagcag tacaacagca cgtaccgtgt ggtcagcgtc 1620
ctcaccgtcc tgcaccagga ctggctgaat ggcaaggagt acaagtgcaa ggtctccaac 1680
aaagccctcg gcgcccccat cgagaaaacc atctccaaag ccaaagggca gccccgagaa 1740
ccacaggtgt acaccctgcc cccatgccgg gatgagctga ccaagaacca ggtcagcctg 1800
tggtgcctgg tcaaaggctt ctatcccagc gacatcgccg tggagtggga gagcaatggg 1860
cagccggaga acaactacaa gaccacgcct cccgtgctgg actccgacgg ctccttcttc 1920
ctctacagca agctcaccgt ggacaagagc aggtggcagc aggggaacgt cttctcatgc 1980
tccgtgatgc atgaggctct gcacaaccac tacacgcaga agagcctctc cctgtctccg 2040
ggtaaa 2046

SEQ ID NO: 189
<212> PRT
<223> M4-3 ML2(VL-CL)
Asp Ile Gln Met Thr Gln Ser Pro Ser Ser Leu Ser Ala Ser Val Gly
1 5 10 15
Asp Arg Val Thr Ile Thr Cys Arg Ala Ser Gln Gly Ile Arg Asn Tyr
20 25 30
Leu Asn Trp Tyr Gln Gln Lys Pro Gly Lys Ala Pro Lys Leu Leu Ile
35 40 45
Tyr Tyr Thr Ser Ser Leu His Ser Gly Val Pro Ser Arg Phe Ser Gly
50 55 60
Ser Gly Ser Gly Thr Asp Phe Thr Leu Thr Ile Ser Ser Leu Gln Pro
65 70 75 80
Glu Asp Phe Ala Thr Tyr Tyr Cys Gln Gln Tyr Ser Lys Leu Pro Trp
85 90 95
Thr Phe Gly Gln Gly Thr Lys Val Glu Ile Lys Arg Thr Val Ala Ala
100 105 110
Pro Ser Val Phe Ile Phe Pro Pro Ser Asp Glu Gln Leu Lys Ser Gly
115 120 125
Thr Ala Ser Val Val Cys Leu Leu Asn Asn Phe Tyr Pro Arg Glu Ala
130 135 140
Lys Val Gln Trp Lys Val Asp Asn Ala Leu Gln Ser Gly Asn Ser Gln
145 150 155 160
Glu Ser Val Thr Glu Gln Asp Ser Lys Asp Ser Thr Tyr Ser Leu Ser
165 170 175
Ser Thr Leu Thr Leu Ser Lys Ala Asp Tyr Glu Lys His Lys Val Tyr
180 185 190
Ala Cys Glu Val Thr His Gln Gly Leu Ser Ser Pro Val Thr Lys Ser
195 200 205
Phe Asn Arg Gly Glu Cys
210

SEQ ID NO: 190
<212> DNA
<223> M4-3 ML2(VL-CL)
gacatccaga tgacccagag ccccagcagc ctgagcgcca gcgtgggcga cagagtgacc 60
atcacctgcc gggccagcca gggcatccgg aactacctga actggtatca gcagaagccc 120
ggcaaggccc ccaagctgct gatctactac accagcagcc tgcacagcgg cgtgcctagc 180
cggtttagcg gcagcggctc cggcaccgac ttcaccctga ccattagctc cctgcagccc 240
gaggacttcg ccacctacta ctgccagcag tacagcaagc tgccctggac cttcggccag 300
ggaacaaagg tggagatcaa gcgtacggtg gctgcaccat ctgtcttcat cttcccgcca 360
tctgatgagc agttgaaatc tggaactgcc tctgttgtgt gcctgctgaa taacttctat 420
cccagagagg ccaaagtaca gtggaaggtg gataacgccc tccaatcggg taactcccag 480
gagagtgtca cagagcagga cagcaaggac agcacctaca gcctcagcag caccctgacg 540
ctgagcaaag cagactacga gaaacacaaa gtctacgcct gcgaagtcac ccatcagggc 600
ctgagctcgc ccgtcacaaa gagcttcaac aggggagagt gt 642

SEQ ID NO: 191
<212> PRT
<223> V9(VL-CH1)-M4-3 ML2(VH-CH1)-Fc(knob) P329G LALA
Asp Ile Gln Met Thr Gln Ser Pro Ser Ser Leu Ser Ala Ser Val Gly
1 5 10 15
Asp Arg Val Thr Ile Thr Cys Arg Ala Ser Gln Asp Ile Arg Asn Tyr
20 25 30
Leu Asn Trp Tyr Gln Gln Lys Pro Gly Lys Ala Pro Lys Leu Leu Ile
35 40 45
Tyr Tyr Thr Ser Arg Leu Glu Ser Gly Val Pro Ser Arg Phe Ser Gly
50 55 60
Ser Gly Ser Gly Thr Asp Tyr Thr Leu Thr Ile Ser Ser Leu Gln Pro
65 70 75 80
Glu Asp Phe Ala Thr Tyr Tyr Cys Gln Gln Gly Asn Thr Leu Pro Trp
85 90 95
Thr Phe Gly Gln Gly Thr Lys Val Glu Ile Lys Ser Ser Ala Ser Thr
100 105 110
Lys Gly Pro Ser Val Phe Pro Leu Ala Pro Ser Ser Lys Ser Thr Ser
115 120 125
Gly Gly Thr Ala Ala Leu Gly Cys Leu Val Lys Asp Tyr Phe Pro Glu
130 135 140
Pro Val Thr Val Ser Trp Asn Ser Gly Ala Leu Thr Ser Gly Val His
145 150 155 160
Thr Phe Pro Ala Val Leu Gln Ser Ser Gly Leu Tyr Ser Leu Ser Ser
165 170 175
Val Val Thr Val Pro Ser Ser Ser Leu Gly Thr Gln Thr Tyr Ile Cys
180 185 190
Asn Val Asn His Lys Pro Ser Asn Thr Lys Val Asp Lys Lys Val Glu
195 200 205
Pro Lys Ser Cys Gly Gly Gly Gly Ser Gly Gly Gly Gly Ser Gln Val
210 215 220
Gln Leu Gln Glu Ser Gly Pro Gly Leu Val Lys Pro Ser Gln Thr Leu
225 230 235 240
Ser Leu Thr Cys Thr Val Ser Gly Gly Ser Ile Thr Ser Gly Tyr Tyr
245 250 255
Trp Asn Trp Ile Arg Gln His Pro Gly Lys Gly Leu Glu Trp Ile Gly
260 265 270
Tyr Ile Thr Tyr Asp Gly Ser Asn Asn Tyr Asn Pro Ser Leu Lys Ser
275 280 285
Arg Val Thr Ile Ser Arg Asp Thr Ser Lys Asn Gln Phe Ser Leu Lys
290 295 300
Leu Ser Ser Val Thr Ala Ala Asp Thr Ala Val Tyr Tyr Cys Ala Asp
305 310 315 320
Phe Asp Tyr Trp Gly Gln Gly Thr Leu Val Thr Val Ser Ser Ala Ser
325 330 335
Thr Lys Gly Pro Ser Val Phe Pro Leu Ala Pro Ser Ser Lys Ser Thr
340 345 350
Ser Gly Gly Thr Ala Ala Leu Gly Cys Leu Val Lys Asp Tyr Phe Pro
355 360 365
Glu Pro Val Thr Val Ser Trp Asn Ser Gly Ala Leu Thr Ser Gly Val
370 375 380
His Thr Phe Pro Ala Val Leu Gln Ser Ser Gly Leu Tyr Ser Leu Ser
385 390 395 400
Ser Val Val Thr Val Pro Ser Ser Ser Leu Gly Thr Gln Thr Tyr Ile
405 410 415
Cys Asn Val Asn His Lys Pro Ser Asn Thr Lys Val Asp Lys Lys Val
420 425 430
Glu Pro Lys Ser Cys Asp Lys Thr His Thr Cys Pro Pro Cys Pro Ala
435 440 445
Pro Glu Ala Ala Gly Gly Pro Ser Val Phe Leu Phe Pro Pro Lys Pro
450 455 460
Lys Asp Thr Leu Met Ile Ser Arg Thr Pro Glu Val Thr Cys Val Val
465 470 475 480
Val Asp Val Ser His Glu Asp Pro Glu Val Lys Phe Asn Trp Tyr Val
485 490 495
Asp Gly Val Glu Val His Asn Ala Lys Thr Lys Pro Arg Glu Glu Gln
500 505 510
Tyr Asn Ser Thr Tyr Arg Val Val Ser Val Leu Thr Val Leu His Gln
515 520 525
Asp Trp Leu Asn Gly Lys Glu Tyr Lys Cys Lys Val Ser Asn Lys Ala
530 535 540
Leu Gly Ala Pro Ile Glu Lys Thr Ile Ser Lys Ala Lys Gly Gln Pro
545 550 555 560
Arg Glu Pro Gln Val Tyr Thr Leu Pro Pro Cys Arg Asp Glu Leu Thr
565 570 575
Lys Asn Gln Val Ser Leu Trp Cys Leu Val Lys Gly Phe Tyr Pro Ser
580 585 590
Asp Ile Ala Val Glu Trp Glu Ser Asn Gly Gln Pro Glu Asn Asn Tyr
595 600 605
Lys Thr Thr Pro Pro Val Leu Asp Ser Asp Gly Ser Phe Phe Leu Tyr
610 615 620
Ser Lys Leu Thr Val Asp Lys Ser Arg Trp Gln Gln Gly Asn Val Phe
625 630 635 640
Ser Cys Ser Val Met His Glu Ala Leu His Asn His Tyr Thr Gln Lys
645 650 655
Ser Leu Ser Leu Ser Pro Gly Lys
660

SEQ ID NO: 192
<212> DNA
<223> V9(VL-CH1)-M4-3 ML2(VH-CH1)-Fc(knob) P329G LALA (DNA)
gatatccaga tgacccagag ccccagctct ctgagcgcca gcgtgggcga cagagtgacc 60
atcacctgtc gggccagcca ggacatcaga aactacctga actggtatca gcagaagccc 120
ggcaaggccc ccaagctgct gatctactac accagcagac tggaaagcgg cgtgccctcc 180
agattttccg gcagcggctc cggcaccgac tacaccctga ccatcagcag cctgcagccc 240
gaggatttcg ccacatatta ctgccagcag ggcaataccc tgccctggac cttcggacag 300
ggcacaaaag tggaaatcaa gagcagcgct tccaccaaag gcccttccgt gtttcctctg 360
gctcctagct ccaagtccac ctctggaggc accgctgctc tcggatgcct cgtgaaggat 420
tattttcctg agcctgtgac agtgtcctgg aatagcggag cactgacctc tggagtgcat 480
actttccccg ctgtgctgca gtcctctgga ctgtacagcc tgagcagcgt ggtgacagtg 540
cccagcagca gcctgggcac ccagacctac atctgcaacg tgaaccacaa gcccagcaac 600
accaaggtgg acaagaaggt ggaacccaag tcttgtggcg gaggcggatc cggcggaggg 660
ggatctcagg tgcagctgca ggaaagcggc cctggcctgg tcaagcccag ccagaccctg 720
agcctgacct gcaccgtgtc cggcggcagc atcaccagcg gctactactg gaactggatt 780
cggcagcacc ccggcaaggg cctggaatgg atcggctaca tcacctacga cggcagcaac 840
aactacaacc ccagcctgaa gtccagagtg accatcagcc gggacaccag caagaaccag 900
ttcagcctga agctgtccag cgtgacagcc gccgacaccg ccgtgtacta ctgcgccgac 960
ttcgactact ggggccaggg caccctggtc accgtgtcca gcgctagcac caagggcccc 1020
agcgtgttcc ccctggcacc cagcagcaag agcacatctg gcggaacagc cgctctgggc 1080
tgtctggtga aagactactt ccccgagccc gtgaccgtgt cttggaactc tggcgccctg 1140
accagcggcg tgcacacctt tccagccgtg ctgcagagca gcggcctgta ctccctgtcc 1200
tccgtggtca ccgtgccctc tagctccctg ggaacacaga catatatctg taatgtcaat 1260
cacaagcctt ccaacaccaa agtcgataag aaagtcgagc ccaagagctg cgacaaaact 1320
cacacatgcc caccgtgccc agcacctgaa gctgcagggg gaccgtcagt cttcctcttc 1380
cccccaaaac ccaaggacac cctcatgatc tcccggaccc ctgaggtcac atgcgtggtg 1440
gtggacgtga gccacgaaga ccctgaggtc aagttcaact ggtacgtgga cggcgtggag 1500
gtgcataatg ccaagacaaa gccgcgggag gagcagtaca acagcacgta ccgtgtggtc 1560
agcgtcctca ccgtcctgca ccaggactgg ctgaatggca aggagtacaa gtgcaaggtc 1620
tccaacaaag ccctcggcgc ccccatcgag aaaaccatct ccaaagccaa agggcagccc 1680
cgagaaccac aggtgtacac cctgccccca tgccgggatg agctgaccaa gaaccaggtc 1740
agcctgtggt gcctggtcaa aggcttctat cccagcgaca tcgccgtgga gtgggagagc 1800
aatgggcagc cggagaacaa ctacaagacc acgcctcccg tgctggactc cgacggctcc 1860
ttcttcctct acagcaagct caccgtggac aagagcaggt ggcagcaggg gaacgtcttc 1920
tcatgctccg tgatgcatga ggctctgcac aaccactaca cgcagaagag cctctccctg 1980
tctccgggta aa 1992

SEQ ID NO: 193
<212> PRT
<223> M4-3 ML2(VH-CH1)-Fc(hole) P329G LALA
Gln Val Gln Leu Gln Glu Ser Gly Pro Gly Leu Val Lys Pro Ser Gln
1 5 10 15
Thr Leu Ser Leu Thr Cys Thr Val Ser Gly Gly Ser Ile Thr Ser Gly
20 25 30
Tyr Tyr Trp Asn Trp Ile Arg Gln His Pro Gly Lys Gly Leu Glu Trp
35 40 45
Ile Gly Tyr Ile Thr Tyr Asp Gly Ser Asn Asn Tyr Asn Pro Ser Leu
50 55 60
Lys Ser Arg Val Thr Ile Ser Arg Asp Thr Ser Lys Asn Gln Phe Ser
65 70 75 80
Leu Lys Leu Ser Ser Val Thr Ala Ala Asp Thr Ala Val Tyr Tyr Cys
85 90 95
Ala Asp Phe Asp Tyr Trp Gly Gln Gly Thr Leu Val Thr Val Ser Ser
100 105 110
Ala Ser Thr Lys Gly Pro Ser Val Phe Pro Leu Ala Pro Ser Ser Lys
115 120 125
Ser Thr Ser Gly Gly Thr Ala Ala Leu Gly Cys Leu Val Lys Asp Tyr
130 135 140
Phe Pro Glu Pro Val Thr Val Ser Trp Asn Ser Gly Ala Leu Thr Ser
145 150 155 160
Gly Val His Thr Phe Pro Ala Val Leu Gln Ser Ser Gly Leu Tyr Ser
165 170 175
Leu Ser Ser Val Val Thr Val Pro Ser Ser Ser Leu Gly Thr Gln Thr
180 185 190
Tyr Ile Cys Asn Val Asn His Lys Pro Ser Asn Thr Lys Val Asp Lys
195 200 205
Lys Val Glu Pro Lys Ser Cys Asp Lys Thr His Thr Cys Pro Pro Cys
210 215 220
Pro Ala Pro Glu Ala Ala Gly Gly Pro Ser Val Phe Leu Phe Pro Pro
225 230 235 240
Lys Pro Lys Asp Thr Leu Met Ile Ser Arg Thr Pro Glu Val Thr Cys
245 250 255
Val Val Val Asp Val Ser His Glu Asp Pro Glu Val Lys Phe Asn Trp
260 265 270
Tyr Val Asp Gly Val Glu Val His Asn Ala Lys Thr Lys Pro Arg Glu
275 280 285
Glu Gln Tyr Asn Ser Thr Tyr Arg Val Val Ser Val Leu Thr Val Leu
290 295 300
His Gln Asp Trp Leu Asn Gly Lys Glu Tyr Lys Cys Lys Val Ser Asn
305 310 315 320
Lys Ala Leu Gly Ala Pro Ile Glu Lys Thr Ile Ser Lys Ala Lys Gly
325 330 335
Gln Pro Arg Glu Pro Gln Val Cys Thr Leu Pro Pro Ser Arg Asp Glu
340 345 350
Leu Thr Lys Asn Gln Val Ser Leu Ser Cys Ala Val Lys Gly Phe Tyr
355 360 365
Pro Ser Asp Ile Ala Val Glu Trp Glu Ser Asn Gly Gln Pro Glu Asn
370 375 380
Asn Tyr Lys Thr Thr Pro Pro Val Leu Asp Ser Asp Gly Ser Phe Phe
385 390 395 400
Leu Val Ser Lys Leu Thr Val Asp Lys Ser Arg Trp Gln Gln Gly Asn
405 410 415
Val Phe Ser Cys Ser Val Met His Glu Ala Leu His Asn His Tyr Thr
420 425 430
Gln Lys Ser Leu Ser Leu Ser Pro Gly Lys
435 440

SEQ ID NO: 194
<212> DNA
<223> M4-3 ML2(VH-CH1)-Fc(hole) P329G LALA
caggtgcagc tgcaggaaag cggccctggc ctggtcaagc ccagccagac cctgagcctg 60
acctgcaccg tgtccggcgg cagcatcacc agcggctact actggaactg gatccggcag 120
caccccggca agggcctgga atggatcggc tacatcacct acgacggcag caacaactac 180
aaccccagcc tgaagtccag agtgaccatc agccgggaca ccagcaagaa ccagttcagc 240
ctgaagctgt ccagcgtgac agccgccgac accgccgtgt actactgcgc cgacttcgac 300
tactggggcc agggcaccct ggtcaccgtg tccagcgcta gcaccaaggg cccctccgtg 360
ttccccctgg cccccagcag caagagcacc agcggcggca cagccgctct gggctgcctg 420
gtcaaggact acttccccga gcccgtgacc gtgtcctgga acagcggagc cctgacctcc 480
ggcgtgcaca ccttccccgc cgtgctgcag agttctggcc tgtatagcct gagcagcgtg 540
gtcaccgtgc cttctagcag cctgggcacc cagacctaca tctgcaacgt gaaccacaag 600
cccagcaaca ccaaggtgga caagaaggtg gagcccaaga gctgcgacaa aactcacaca 660
tgcccaccgt gcccagcacc tgaagctgca gggggaccgt cagtcttcct cttcccccca 720
aaacccaagg acaccctcat gatctcccgg acccctgagg tcacatgcgt ggtggtggac 780
gtgagccacg aagaccctga ggtcaagttc aactggtacg tggacggcgt ggaggtgcat 840
aatgccaaga caaagccgcg ggaggagcag tacaacagca cgtaccgtgt ggtcagcgtc 900
ctcaccgtcc tgcaccagga ctggctgaat ggcaaggagt acaagtgcaa ggtctccaac 960
aaagccctcg gcgcccccat cgagaaaacc atctccaaag ccaaagggca gccccgagaa 1020
ccacaggtgt gcaccctgcc cccatcccgg gatgagctga ccaagaacca ggtcagcctc 1080
tcgtgcgcag tcaaaggctt ctatcccagc gacatcgccg tggagtggga gagcaatggg 1140
cagccggaga acaactacaa gaccacgcct cccgtgctgg actccgacgg ctccttcttc 1200
ctcgtgagca agctcaccgt ggacaagagc aggtggcagc aggggaacgt cttctcatgc 1260
tccgtgatgc atgaggctct gcacaaccac tacacgcaga agagcctctc cctgtctccg 1320
ggtaaa 1326

SEQ ID NO: 195
<212> PRT
<223> V9(VH-CL)-M4-3 ML2(VH-CH1)-Fc(knob) P329G LALA
Glu Val Gln Leu Val Glu Ser Gly Gly Gly Leu Val Gln Pro Gly Gly
1 5 10 15
Ser Leu Arg Leu Ser Cys Ala Ala Ser Gly Tyr Ser Phe Thr Gly Tyr
20 25 30
Thr Met Asn Trp Val Arg Gln Ala Pro Gly Lys Gly Leu Glu Trp Val
35 40 45
Ala Leu Ile Asn Pro Tyr Lys Gly Val Ser Thr Tyr Asn Gln Lys Phe
50 55 60
Lys Asp Arg Phe Thr Ile Ser Val Asp Lys Ser Lys Asn Thr Ala Tyr
65 70 75 80
Leu Gln Met Asn Ser Leu Arg Ala Glu Asp Thr Ala Val Tyr Tyr Cys
85 90 95
Ala Arg Ser Gly Tyr Tyr Gly Asp Ser Asp Trp Tyr Phe Asp Val Trp
100 105 110
Gly Gln Gly Thr Leu Val Thr Val Ser Ser Ala Ser Val Ala Ala Pro
115 120 125
Ser Val Phe Ile Phe Pro Pro Ser Asp Glu Gln Leu Lys Ser Gly Thr
130 135 140
Ala Ser Val Val Cys Leu Leu Asn Asn Phe Tyr Pro Arg Glu Ala Lys
145 150 155 160
Val Gln Trp Lys Val Asp Asn Ala Leu Gln Ser Gly Asn Ser Gln Glu
165 170 175
Ser Val Thr Glu Gln Asp Ser Lys Asp Ser Thr Tyr Ser Leu Ser Ser
180 185 190
Thr Leu Thr Leu Ser Lys Ala Asp Tyr Glu Lys His Lys Val Tyr Ala
195 200 205
Cys Glu Val Thr His Gln Gly Leu Ser Ser Pro Val Thr Lys Ser Phe
210 215 220
Asn Arg Gly Glu Cys Gly Gly Gly Gly Ser Gly Gly Gly Gly Ser Gln
225 230 235 240
Val Gln Leu Gln Glu Ser Gly Pro Gly Leu Val Lys Pro Ser Gln Thr
245 250 255
Leu Ser Leu Thr Cys Thr Val Ser Gly Gly Ser Ile Thr Ser Gly Tyr
260 265 270
Tyr Trp Asn Trp Ile Arg Gln His Pro Gly Lys Gly Leu Glu Trp Ile
275 280 285
Gly Tyr Ile Thr Tyr Asp Gly Ser Asn Asn Tyr Asn Pro Ser Leu Lys
290 295 300
Ser Arg Val Thr Ile Ser Arg Asp Thr Ser Lys Asn Gln Phe Ser Leu
305 310 315 320
Lys Leu Ser Ser Val Thr Ala Ala Asp Thr Ala Val Tyr Tyr Cys Ala
325 330 335
Asp Phe Asp Tyr Trp Gly Gln Gly Thr Leu Val Thr Val Ser Ser Ala
340 345 350
Ser Thr Lys Gly Pro Ser Val Phe Pro Leu Ala Pro Ser Ser Lys Ser
355 360 365
Thr Ser Gly Gly Thr Ala Ala Leu Gly Cys Leu Val Lys Asp Tyr Phe
370 375 380
Pro Glu Pro Val Thr Val Ser Trp Asn Ser Gly Ala Leu Thr Ser Gly
385 390 395 400
Val His Thr Phe Pro Ala Val Leu Gln Ser Ser Gly Leu Tyr Ser Leu
405 410 415
Ser Ser Val Val Thr Val Pro Ser Ser Ser Leu Gly Thr Gln Thr Tyr
420 425 430
Ile Cys Asn Val Asn His Lys Pro Ser Asn Thr Lys Val Asp Lys Lys
435 440 445
Val Glu Pro Lys Ser Cys Asp Lys Thr His Thr Cys Pro Pro Cys Pro
450 455 460
Ala Pro Glu Ala Ala Gly Gly Pro Ser Val Phe Leu Phe Pro Pro Lys
465 470 475 480
Pro Lys Asp Thr Leu Met Ile Ser Arg Thr Pro Glu Val Thr Cys Val
485 490 495
Val Val Asp Val Ser His Glu Asp Pro Glu Val Lys Phe Asn Trp Tyr
500 505 510
Val Asp Gly Val Glu Val His Asn Ala Lys Thr Lys Pro Arg Glu Glu
515 520 525
Gln Tyr Asn Ser Thr Tyr Arg Val Val Ser Val Leu Thr Val Leu His
530 535 540
Gln Asp Trp Leu Asn Gly Lys Glu Tyr Lys Cys Lys Val Ser Asn Lys
545 550 555 560
Ala Leu Gly Ala Pro Ile Glu Lys Thr Ile Ser Lys Ala Lys Gly Gln
565 570 575
Pro Arg Glu Pro Gln Val Tyr Thr Leu Pro Pro Cys Arg Asp Glu Leu
580 585 590
Thr Lys Asn Gln Val Ser Leu Trp Cys Leu Val Lys Gly Phe Tyr Pro
595 600 605
Ser Asp Ile Ala Val Glu Trp Glu Ser Asn Gly Gln Pro Glu Asn Asn
610 615 620
Tyr Lys Thr Thr Pro Pro Val Leu Asp Ser Asp Gly Ser Phe Phe Leu
625 630 635 640
Tyr Ser Lys Leu Thr Val Asp Lys Ser Arg Trp Gln Gln Gly Asn Val
645 650 655
Phe Ser Cys Ser Val Met His Glu Ala Leu His Asn His Tyr Thr Gln
660 665 670
Lys Ser Leu Ser Leu Ser Pro Gly Lys
675 680

SEQ ID NO: 196
<212> DNA
<223> V9(VH-CL)-M4-3 ML2(VH-CH1)-Fc(knob) P329G LALA (DNA)
gaggtgcagc tggtcgagag cggaggcggc ctggtgcagc ctggcggcag cctgagactg 60
agctgcgccg ccagcggcta cagcttcacc ggctacacca tgaactgggt ccggcaggca 120
cctggcaagg gactggaatg ggtggccctg atcaacccct acaagggcgt gagcacctac 180
aaccagaagt tcaaggaccg gttcaccatc agcgtggaca agagcaagaa caccgcctat 240
ctgcagatga acagcctgcg ggccgaggac accgccgtgt actactgcgc cagaagcggc 300
tactacggcg acagcgactg gtacttcgac gtgtggggcc agggcaccct cgtgaccgtg 360
tctagcgcta gcgtggctgc accatctgtc ttcatcttcc cgccatctga tgagcagttg 420
aaatctggaa ctgcctctgt tgtgtgcctg ctgaataact tctatcccag agaggccaaa 480
gtacagtgga aggtggataa cgccctccaa tcgggtaact cccaggagag tgtcacagag 540
caggacagca aggacagcac ctacagcctc agcagcaccc tgacgctgag caaagcagac 600
tacgagaaac acaaagtcta cgcctgcgaa gtcacccatc agggcctgag ctcgcccgtc 660
acaaagagct tcaacagggg agagtgtggc ggaggcggat ccggcggagg gggatctcag 720
gtgcagctgc aggaaagcgg ccctggcctg gtcaagccca gccagaccct gagcctgacc 780
tgcaccgtgt ccggcggcag catcaccagc ggctactact ggaactggat tcggcagcac 840
cccggcaagg gcctggaatg gatcggctac atcacctacg acggcagcaa caactacaac 900
cccagcctga agtccagagt gaccatcagc cgggacacca gcaagaacca gttcagcctg 960
aagctgtcca gcgtgacagc cgccgacacc gccgtgtact actgcgccga cttcgactac 1020
tggggccagg gcaccctggt caccgtgtcc agcgctagca ccaagggccc cagcgtgttc 1080
cccctggcac ccagcagcaa gagcacatct ggcggaacag ccgctctggg ctgtctggtg 1140
aaagactact tccccgagcc cgtgaccgtg tcttggaact ctggcgccct gaccagcggc 1200
gtgcacacct ttccagccgt gctgcagagc agcggcctgt actccctgtc ctccgtggtc 1260
accgtgccct ctagctccct gggaacacag acatatatct gtaatgtcaa tcacaagcct 1320
tccaacacca aagtcgataa gaaagtcgag cccaagagct gcgacaaaac tcacacatgc 1380
ccaccgtgcc cagcacctga agctgcaggg ggaccgtcag tcttcctctt ccccccaaaa 1440
cccaaggaca ccctcatgat ctcccggacc cctgaggtca catgcgtggt ggtggacgtg 1500
agccacgaag accctgaggt caagttcaac tggtacgtgg acggcgtgga ggtgcataat 1560
gccaagacaa agccgcggga ggagcagtac aacagcacgt accgtgtggt cagcgtcctc 1620
accgtcctgc accaggactg gctgaatggc aaggagtaca agtgcaaggt ctccaacaaa 1680
gccctcggcg cccccatcga gaaaaccatc tccaaagcca aagggcagcc ccgagaacca 1740
caggtgtaca ccctgccccc atgccgggat gagctgacca agaaccaggt cagcctgtgg 1800
tgcctggtca aaggcttcta tcccagcgac atcgccgtgg agtgggagag caatgggcag 1860
ccggagaaca actacaagac cacgcctccc gtgctggact ccgacggctc cttcttcctc 1920
tacagcaagc tcaccgtgga caagagcagg tggcagcagg ggaacgtctt ctcatgctcc 1980
gtgatgcatg aggctctgca caaccactac acgcagaaga gcctctccct gtctccgggt 2040
aaa 2043

SEQ ID NO: 197
<212> PRT
<223> CH1A1A(VH-CH1)- V9(VH-CL)-Fc(knob) P329G LALA
Gln Val Gln Leu Val Gln Ser Gly Ala Glu Val Lys Lys Pro Gly Ala
1 5 10 15
Ser Val Lys Val Ser Cys Lys Ala Ser Gly Tyr Thr Phe Thr Glu Phe
20 25 30
Gly Met Asn Trp Val Arg Gln Ala Pro Gly Gln Gly Leu Glu Trp Met
35 40 45
Gly Trp Ile Asn Thr Lys Thr Gly Glu Ala Thr Tyr Val Glu Glu Phe
50 55 60
Lys Gly Arg Val Thr Phe Thr Thr Asp Thr Ser Thr Ser Thr Ala Tyr
65 70 75 80
Met Glu Leu Arg Ser Leu Arg Ser Asp Asp Thr Ala Val Tyr Tyr Cys
85 90 95
Ala Arg Trp Asp Phe Ala Tyr Tyr Val Glu Ala Met Asp Tyr Trp Gly
100 105 110
Gln Gly Thr Thr Val Thr Val Ser Ser Ala Ser Thr Lys Gly Pro Ser
115 120 125
Val Phe Pro Leu Ala Pro Ser Ser Lys Ser Thr Ser Gly Gly Thr Ala
130 135 140
Ala Leu Gly Cys Leu Val Lys Asp Tyr Phe Pro Glu Pro Val Thr Val
145 150 155 160
Ser Trp Asn Ser Gly Ala Leu Thr Ser Gly Val His Thr Phe Pro Ala
165 170 175
Val Leu Gln Ser Ser Gly Leu Tyr Ser Leu Ser Ser Val Val Thr Val
180 185 190
Pro Ser Ser Ser Leu Gly Thr Gln Thr Tyr Ile Cys Asn Val Asn His
195 200 205
Lys Pro Ser Asn Thr Lys Val Asp Lys Lys Val Glu Pro Lys Ser Cys
210 215 220
Gly Gly Gly Gly Ser Gly Gly Gly Gly Ser Glu Val Gln Leu Val Glu
225 230 235 240
Ser Gly Gly Gly Leu Val Gln Pro Gly Gly Ser Leu Arg Leu Ser Cys
245 250 255
Ala Ala Ser Gly Tyr Ser Phe Thr Gly Tyr Thr Met Asn Trp Val Arg
260 265 270
Gln Ala Pro Gly Lys Gly Leu Glu Trp Val Ala Leu Ile Asn Pro Tyr
275 280 285
Lys Gly Val Ser Thr Tyr Asn Gln Lys Phe Lys Asp Arg Phe Thr Ile
290 295 300
Ser Val Asp Lys Ser Lys Asn Thr Ala Tyr Leu Gln Met Asn Ser Leu
305 310 315 320
Arg Ala Glu Asp Thr Ala Val Tyr Tyr Cys Ala Arg Ser Gly Tyr Tyr
325 330 335
Gly Asp Ser Asp Trp Tyr Phe Asp Val Trp Gly Gln Gly Thr Leu Val
340 345 350
Thr Val Ser Ser Ala Ser Val Ala Ala Pro Ser Val Phe Ile Phe Pro
355 360 365
Pro Ser Asp Glu Gln Leu Lys Ser Gly Thr Ala Ser Val Val Cys Leu
370 375 380
Leu Asn Asn Phe Tyr Pro Arg Glu Ala Lys Val Gln Trp Lys Val Asp
385 390 395 400
Asn Ala Leu Gln Ser Gly Asn Ser Gln Glu Ser Val Thr Glu Gln Asp
405 410 415
Ser Lys Asp Ser Thr Tyr Ser Leu Ser Ser Thr Leu Thr Leu Ser Lys
420 425 430
Ala Asp Tyr Glu Lys His Lys Val Tyr Ala Cys Glu Val Thr His Gln
435 440 445
Gly Leu Ser Ser Pro Val Thr Lys Ser Phe Asn Arg Gly Glu Cys Asp
450 455 460
Lys Thr His Thr Cys Pro Pro Cys Pro Ala Pro Glu Ala Ala Gly Gly
465 470 475 480
Pro Ser Val Phe Leu Phe Pro Pro Lys Pro Lys Asp Thr Leu Met Ile
485 490 495
Ser Arg Thr Pro Glu Val Thr Cys Val Val Val Asp Val Ser His Glu
500 505 510
Asp Pro Glu Val Lys Phe Asn Trp Tyr Val Asp Gly Val Glu Val His
515 520 525
Asn Ala Lys Thr Lys Pro Arg Glu Glu Gln Tyr Asn Ser Thr Tyr Arg
530 535 540
Val Val Ser Val Leu Thr Val Leu His Gln Asp Trp Leu Asn Gly Lys
545 550 555 560
Glu Tyr Lys Cys Lys Val Ser Asn Lys Ala Leu Gly Ala Pro Ile Glu
565 570 575
Lys Thr Ile Ser Lys Ala Lys Gly Gln Pro Arg Glu Pro Gln Val Tyr
580 585 590
Thr Leu Pro Pro Cys Arg Asp Glu Leu Thr Lys Asn Gln Val Ser Leu
595 600 605
Trp Cys Leu Val Lys Gly Phe Tyr Pro Ser Asp Ile Ala Val Glu Trp
610 615 620
Glu Ser Asn Gly Gln Pro Glu Asn Asn Tyr Lys Thr Thr Pro Pro Val
625 630 635 640
Leu Asp Ser Asp Gly Ser Phe Phe Leu Tyr Ser Lys Leu Thr Val Asp
645 650 655
Lys Ser Arg Trp Gln Gln Gly Asn Val Phe Ser Cys Ser Val Met His
660 665 670
Glu Ala Leu His Asn His Tyr Thr Gln Lys Ser Leu Ser Leu Ser Pro
675 680 685
Gly Lys
690

SEQ ID NO: 198
<212> DNA
<223> CH1A1A(VH-CH1)-V9(VH-CL)-Fc(knob) P329G LALA
caggtgcagc tggtgcagtc tggcgccgaa gtgaagaaac ctggcgccag cgtgaaggtg 60
tcctgcaagg ccagcggcta caccttcacc gagttcggca tgaactgggt ccgacaggcc 120
cctggacagg gcctggaatg gatgggctgg atcaacacca agaccggcga ggccacctac 180
gtggaagagt tcaagggcag agtgaccttc accaccgaca ccagcaccag caccgcctac 240
atggaactgc ggagcctgag aagcgacgac accgccgtgt actactgcgc cagatgggac 300
ttcgcctact atgtggaagc catggactac tggggccagg gcaccaccgt gaccgtgtct 360
agtgctagca caaagggccc cagcgtgttc cctctggccc ctagcagcaa gagcacatct 420
ggcggaacag ccgccctggg ctgcctggtc aaggactact ttcccgagcc cgtgacagtg 480
tcctggaact ctggcgccct gacaagcggc gtgcacacct ttccagccgt gctgcagagc 540
agcggcctgt actctctgag cagcgtggtc accgtgccta gctctagcct gggcacccag 600
acctacatct gcaacgtgaa ccacaagccc agcaacacca aggtggacaa gaaggtggaa 660
cccaagagct gcggcggagg cggatccgga ggcggaggat ccgaagtgca gctggtggaa 720
tctggcggag gcctggtgca gcctggcgga tctctgagac tgagctgtgc cgccagcggc 780
tacagcttca ccggctacac catgaactgg gtgcgccagg cccctggcaa gggactggaa 840
tgggtggccc tgatcaaccc ctacaagggc gtgtccacat acaaccagaa gttcaaggac 900
cggttcacca tcagcgtgga caagagcaag aacaccgcct acctgcagat gaacagcctg 960
cgggccgagg acaccgccgt gtactattgt gccagaagcg gctactacgg cgacagcgac 1020
tggtacttcg acgtgtgggg ccagggcaca ctcgtgaccg tgtcaagcgc tagcgtggcc 1080
gctccctccg tgtttatctt tcccccatcc gatgaacagc tgaaaagcgg caccgcctcc 1140
gtcgtgtgtc tgctgaacaa tttttaccct agggaagcta aagtgcagtg gaaagtggat 1200
aacgcactgc agtccggcaa ctcccaggaa tctgtgacag aacaggactc caaggacagc 1260
acctactccc tgtcctccac cctgacactg tctaaggctg attatgagaa acacaaagtc 1320
tacgcctgcg aagtcaccca tcagggcctg agctcgcccg tcacaaagag cttcaacagg 1380
ggagagtgtg acaagaccca cacctgtccc ccttgtcctg cccctgaagc tgctggcggc 1440
ccttctgtgt tcctgttccc cccaaagccc aaggacaccc tgatgatcag ccggaccccc 1500
gaagtgacct gcgtggtggt ggatgtgtcc cacgaggacc ctgaagtgaa gttcaattgg 1560
tacgtggacg gcgtggaagt gcacaacgcc aagacaaagc cgcgggagga gcagtacaac 1620
agcacgtacc gtgtggtcag cgtcctcacc gtcctgcacc aggactggct gaatggcaag 1680
gagtacaagt gcaaggtctc caacaaagcc ctcggcgccc ccatcgagaa aaccatctcc 1740
aaagccaaag ggcagccccg agaaccacag gtgtacaccc tgcccccatg ccgggatgag 1800
ctgaccaaga accaggtcag cctgtggtgc ctggtcaaag gcttctatcc cagcgacatc 1860
gccgtggagt gggagagcaa tgggcagccg gagaacaact acaagaccac gcctcccgtg 1920
ctggactccg acggctcctt cttcctctac agcaagctca ccgtggacaa gagcaggtgg 1980
cagcagggga acgtcttctc atgctccgtg atgcatgagg ctctgcacaa ccactacacg 2040
cagaagagcc tctccctgtc tccgggtaaa 2070

SEQ ID NO: 199
<212> PRT
<223> H2C(VL-CH1)
Gln Thr Val Val Thr Gln Glu Pro Ser Leu Thr Val Ser Pro Gly Gly
1 5 10 15
Thr Val Thr Leu Thr Cys Gly Ser Ser Thr Gly Ala Val Thr Ser Gly
20 25 30
Tyr Tyr Pro Asn Trp Val Gln Gln Lys Pro Gly Gln Ala Pro Arg Gly
35 40 45
Leu Ile Gly Gly Thr Lys Phe Leu Ala Pro Gly Thr Pro Ala Arg Phe
50 55 60
Ser Gly Ser Leu Leu Gly Gly Lys Ala Ala Leu Thr Leu Ser Gly Val
65 70 75 80
Gln Pro Glu Asp Glu Ala Glu Tyr Tyr Cys Ala Leu Trp Tyr Ser Asn
85 90 95
Arg Trp Val Phe Gly Gly Gly Thr Lys Leu Thr Val Leu Ser Ser Ala
100 105 110
Ser Thr Lys Gly Pro Ser Val Phe Pro Leu Ala Pro Ser Ser Lys Ser
115 120 125
Thr Ser Gly Gly Thr Ala Ala Leu Gly Cys Leu Val Lys Asp Tyr Phe
130 135 140
Pro Glu Pro Val Thr Val Ser Trp Asn Ser Gly Ala Leu Thr Ser Gly
145 150 155 160
Val His Thr Phe Pro Ala Val Leu Gln Ser Ser Gly Leu Tyr Ser Leu
165 170 175
Ser Ser Val Val Thr Val Pro Ser Ser Ser Leu Gly Thr Gln Thr Tyr
180 185 190
Ile Cys Asn Val Asn His Lys Pro Ser Asn Thr Lys Val Asp Lys Lys
195 200 205
Val Glu Pro Lys Ser Cys
210

SEQ ID NO: 200
<212> DNA
<223> H2C(VL-CH1)
cagaccgtgg tgacacagga acccagcctg accgtctccc ctggcggcac cgtgaccctg 60
acctgtggaa gcagcacagg cgccgtgacc agcggctact accccaactg ggtgcagcag 120
aagcccggcc aggcccctag aggactgatc ggcggcacca agtttctggc ccctggcacc 180
cccgccagat tctctggctc tctgctgggc ggcaaggccg ccctgacact gtctggcgtg 240
cagcctgagg acgaggccga gtactactgc gccctgtggt acagcaacag atgggtgttc 300
ggcggaggca ccaagctgac cgtgctgagc agcgcttcca ccaaaggccc ttccgtgttt 360
cctctggctc ctagctccaa gtccacctct ggaggcaccg ctgctctcgg atgcctcgtg 420
aaggattatt ttcctgagcc tgtgacagtg tcctggaata gcggagcact gacctctgga 480
gtgcatactt tccccgctgt gctgcagtcc tctggactgt acagcctgag cagcgtggtg 540
acagtgccca gcagcagcct gggcacccag acctacatct gcaacgtgaa ccacaagccc 600
agcaacacca aggtggacaa gaaggtggaa cccaagtctt gt 642

SEQ ID NO: 201
<212> PRT
<223> H2C(VH-CL)-M4-3 ML2(VH-CH1)-Fc(knob) P329G LALA
Glu Val Gln Leu Val Glu Ser Gly Gly Gly Leu Val Gln Pro Gly Gly
1 5 10 15
Ser Leu Lys Leu Ser Cys Ala Ala Ser Gly Phe Thr Phe Asn Lys Tyr
20 25 30
Ala Met Asn Trp Val Arg Gln Ala Pro Gly Lys Gly Leu Glu Trp Val
35 40 45
Ala Arg Ile Arg Ser Lys Tyr Asn Asn Tyr Ala Thr Tyr Tyr Ala Asp
50 55 60
Ser Val Lys Asp Arg Phe Thr Ile Ser Arg Asp Asp Ser Lys Asn Thr
65 70 75 80
Ala Tyr Leu Gln Met Asn Asn Leu Lys Thr Glu Asp Thr Ala Val Tyr
85 90 95
Tyr Cys Val Arg His Gly Asn Phe Gly Asn Ser Tyr Ile Ser Tyr Trp
100 105 110
Ala Tyr Trp Gly Gln Gly Thr Leu Val Thr Val Ser Ser Ala Ser Val
115 120 125
Ala Ala Pro Ser Val Phe Ile Phe Pro Pro Ser Asp Glu Gln Leu Lys
130 135 140
Ser Gly Thr Ala Ser Val Val Cys Leu Leu Asn Asn Phe Tyr Pro Arg
145 150 155 160
Glu Ala Lys Val Gln Trp Lys Val Asp Asn Ala Leu Gln Ser Gly Asn
165 170 175
Ser Gln Glu Ser Val Thr Glu Gln Asp Ser Lys Asp Ser Thr Tyr Ser
180 185 190
Leu Ser Ser Thr Leu Thr Leu Ser Lys Ala Asp Tyr Glu Lys His Lys
195 200 205
Val Tyr Ala Cys Glu Val Thr His Gln Gly Leu Ser Ser Pro Val Thr
210 215 220
Lys Ser Phe Asn Arg Gly Glu Cys Gly Gly Gly Gly Ser Gly Gly Gly
225 230 235 240
Gly Ser Gln Val Gln Leu Gln Glu Ser Gly Pro Gly Leu Val Lys Pro
245 250 255
Ser Gln Thr Leu Ser Leu Thr Cys Thr Val Ser Gly Gly Ser Ile Thr
260 265 270
Ser Gly Tyr Tyr Trp Asn Trp Ile Arg Gln His Pro Gly Lys Gly Leu
275 280 285
Glu Trp Ile Gly Tyr Ile Thr Tyr Asp Gly Ser Asn Asn Tyr Asn Pro
290 295 300
Ser Leu Lys Ser Arg Val Thr Ile Ser Arg Asp Thr Ser Lys Asn Gln
305 310 315 320
Phe Ser Leu Lys Leu Ser Ser Val Thr Ala Ala Asp Thr Ala Val Tyr
325 330 335
Tyr Cys Ala Asp Phe Asp Tyr Trp Gly Gln Gly Thr Leu Val Thr Val
340 345 350
Ser Ser Ala Ser Thr Lys Gly Pro Ser Val Phe Pro Leu Ala Pro Ser
355 360 365
Ser Lys Ser Thr Ser Gly Gly Thr Ala Ala Leu Gly Cys Leu Val Lys
370 375 380
Asp Tyr Phe Pro Glu Pro Val Thr Val Ser Trp Asn Ser Gly Ala Leu
385 390 395 400
Thr Ser Gly Val His Thr Phe Pro Ala Val Leu Gln Ser Ser Gly Leu
405 410 415
Tyr Ser Leu Ser Ser Val Val Thr Val Pro Ser Ser Ser Leu Gly Thr
420 425 430
Gln Thr Tyr Ile Cys Asn Val Asn His Lys Pro Ser Asn Thr Lys Val
435 440 445
Asp Lys Lys Val Glu Pro Lys Ser Cys Asp Lys Thr His Thr Cys Pro
450 455 460
Pro Cys Pro Ala Pro Glu Ala Ala Gly Gly Pro Ser Val Phe Leu Phe
465 470 475 480
Pro Pro Lys Pro Lys Asp Thr Leu Met Ile Ser Arg Thr Pro Glu Val
485 490 495
Thr Cys Val Val Val Asp Val Ser His Glu Asp Pro Glu Val Lys Phe
500 505 510
Asn Trp Tyr Val Asp Gly Val Glu Val His Asn Ala Lys Thr Lys Pro
515 520 525
Arg Glu Glu Gln Tyr Asn Ser Thr Tyr Arg Val Val Ser Val Leu Thr
530 535 540
Val Leu His Gln Asp Trp Leu Asn Gly Lys Glu Tyr Lys Cys Lys Val
545 550 555 560
Ser Asn Lys Ala Leu Gly Ala Pro Ile Glu Lys Thr Ile Ser Lys Ala
565 570 575
Lys Gly Gln Pro Arg Glu Pro Gln Val Tyr Thr Leu Pro Pro Cys Arg
580 585 590
Asp Glu Leu Thr Lys Asn Gln Val Ser Leu Trp Cys Leu Val Lys Gly
595 600 605
Phe Tyr Pro Ser Asp Ile Ala Val Glu Trp Glu Ser Asn Gly Gln Pro
610 615 620
Glu Asn Asn Tyr Lys Thr Thr Pro Pro Val Leu Asp Ser Asp Gly Ser
625 630 635 640
Phe Phe Leu Tyr Ser Lys Leu Thr Val Asp Lys Ser Arg Trp Gln Gln
645 650 655
Gly Asn Val Phe Ser Cys Ser Val Met His Glu Ala Leu His Asn His
660 665 670
Tyr Thr Gln Lys Ser Leu Ser Leu Ser Pro Gly Lys
675 680

SEQ ID NO: 202
<212> DNA
<223> H2C(VH-CL)-M4-3 ML2(VH-CH1)-Fc(knob) P329G LALA
gaggtgcagc tggtggaaag cggcggagga ctggtgcagc ctggcggaag cctgaagctg 60
tcttgcgccg ccagcggctt caccttcaac aaatacgcca tgaactgggt gcgccaggcc 120
cctggcaagg gactggaatg ggtggcccgg atcagaagca agtacaacaa ctacgccacc 180
tactacgccg acagcgtgaa ggaccggttc accatcagcc gggacgacag caagaacacc 240
gcctacctgc agatgaacaa cctgaaaacc gaggacaccg ccgtgtacta ctgcgtgcgg 300
cacggcaact tcggcaacag ctacatcagc tactgggcct actggggaca gggcaccctg 360
gtgacagtgt ccagcgctag cgtggctgca ccatctgtct tcatcttccc gccatctgat 420
gagcagttga aatctggaac tgcctctgtt gtgtgcctgc tgaataactt ctatcccaga 480
gaggccaaag tacagtggaa ggtggataac gccctccaat cgggtaactc ccaggagagt 540
gtcacagagc aggacagcaa ggacagcacc tacagcctca gcagcaccct gacgctgagc 600
aaagcagact acgagaaaca caaagtctac gcctgcgaag tcacccatca gggcctgagc 660
tcgcccgtca caaagagctt caacagggga gagtgtggcg gaggcggatc cggcggaggg 720
ggatctcagg tgcagctgca ggaaagcggc cctggcctgg tcaagcccag ccagaccctg 780
agcctgacct gcaccgtgtc cggcggcagc atcaccagcg gctactactg gaactggatt 840
cggcagcacc ccggcaaggg cctggaatgg atcggctaca tcacctacga cggcagcaac 900
aactacaacc ccagcctgaa gtccagagtg accatcagcc gggacaccag caagaaccag 960
ttcagcctga agctgtccag cgtgacagcc gccgacaccg ccgtgtacta ctgcgccgac 1020
ttcgactact ggggccaggg caccctggtc accgtgtcca gcgctagcac caagggcccc 1080
agcgtgttcc ccctggcacc cagcagcaag agcacatctg gcggaacagc cgctctgggc 1140
tgtctggtga aagactactt ccccgagccc gtgaccgtgt cttggaactc tggcgccctg 1200
accagcggcg tgcacacctt tccagccgtg ctgcagagca gcggcctgta ctccctgtcc 1260
tccgtggtca ccgtgccctc tagctccctg ggaacacaga catatatctg taatgtcaat 1320
cacaagcctt ccaacaccaa agtcgataag aaagtcgagc ccaagagctg cgacaaaact 1380
cacacatgcc caccgtgccc agcacctgaa gctgcagggg gaccgtcagt cttcctcttc 1440
cccccaaaac ccaaggacac cctcatgatc tcccggaccc ctgaggtcac atgcgtggtg 1500
gtggacgtga gccacgaaga ccctgaggtc aagttcaact ggtacgtgga cggcgtggag 1560
gtgcataatg ccaagacaaa gccgcgggag gagcagtaca acagcacgta ccgtgtggtc 1620
agcgtcctca ccgtcctgca ccaggactgg ctgaatggca aggagtacaa gtgcaaggtc 1680
tccaacaaag ccctcggcgc ccccatcgag aaaaccatct ccaaagccaa agggcagccc 1740
cgagaaccac aggtgtacac cctgccccca tgccgggatg agctgaccaa gaaccaggtc 1800
agcctgtggt gcctggtcaa aggcttctat cccagcgaca tcgccgtgga gtgggagagc 1860
aatgggcagc cggagaacaa ctacaagacc acgcctcccg tgctggactc cgacggctcc 1920
ttcttcctct acagcaagct caccgtggac aagagcaggt ggcagcaggg gaacgtcttc 1980
tcatgctccg tgatgcatga ggctctgcac aaccactaca cgcagaagag cctctccctg 2040
tctccgggta aa 2052

SEQ ID NO: 203
<212> PRT
<223> 431/26 (VL-CL)
Asp Ile Gln Met Thr Gln Ser Pro Ser Ser Leu Ser Ala Ser Val Gly
1 5 10 15
Asp Arg Val Thr Ile Thr Cys Ser Thr Ser Ser Ser Val Ser Tyr Met
20 25 30
His Trp Tyr Gln Gln Lys Pro Gly Lys Ala Pro Lys Leu Leu Ile Tyr
35 40 45
Ser Thr Ser Asn Leu Ala Ser Gly Val Pro Ser Arg Phe Ser Gly Ser
50 55 60
Gly Ser Gly Thr Asp Phe Thr Phe Thr Ile Ser Ser Leu Gln Pro Glu
65 70 75 80
Asp Ile Ala Thr Tyr Tyr Cys His Gln Trp Ser Ser Tyr Pro Thr Phe
85 90 95
Gly Gln Gly Thr Lys Val Glu Ile Lys Arg Thr Val Ala Ala Pro Ser
100 105 110
Val Phe Ile Phe Pro Pro Ser Asp Glu Gln Leu Lys Ser Gly Thr Ala
115 120 125
Ser Val Val Cys Leu Leu Asn Asn Phe Tyr Pro Arg Glu Ala Lys Val
130 135 140
Gln Trp Lys Val Asp Asn Ala Leu Gln Ser Gly Asn Ser Gln Glu Ser
145 150 155 160
Val Thr Glu Gln Asp Ser Lys Asp Ser Thr Tyr Ser Leu Ser Ser Thr
165 170 175
Leu Thr Leu Ser Lys Ala Asp Tyr Glu Lys His Lys Val Tyr Ala Cys
180 185 190
Glu Val Thr His Gln Gly Leu Ser Ser Pro Val Thr Lys Ser Phe Asn
195 200 205
Arg Gly Glu Cys
210

SEQ ID NO: 204
<212> DNA
<223> 431/26 (VL-CL)
gacatccaga tgacccagag ccccagcagc ctgtctgcca gcgtgggcga cagagtgacc 60
atcacctgta gcaccagcag cagcgtgtcc tacatgcact ggtatcagca gaagcccggc 120
aaggccccca agctgctgat ctacagcacc tccaatctgg ccagcggcgt gcccagcaga 180
ttttctggca gcggctccgg caccgacttc accttcacca tcagctccct gcagcccgag 240
gatatcgcca cctactactg ccaccagtgg tccagctacc ccacctttgg ccagggcacc 300
aaggtggaaa tcaagcgtac ggtggctgca ccatctgtct tcatcttccc gccatctgat 360
gagcagttga aatctggaac tgcctctgtt gtgtgcctgc tgaataactt ctatcccaga 420
gaggccaaag tacagtggaa ggtggataac gccctccaat cgggtaactc ccaggagagt 480
gtcacagagc aggacagcaa ggacagcacc tacagcctca gcagcaccct gacgctgagc 540
aaagcagact acgagaaaca caaagtctac gcctgcgaag tcacccatca gggcctgagc 600
tcgcccgtca caaagagctt caacagggga gagtgt 636

SEQ ID NO: 205
<212> PRT
<223> 431/26 (VH-CH1)-V9(VH-CL)-Fc(knob) P329G LALA
Gln Val Gln Leu Gln Glu Ser Gly Pro Gly Leu Val Arg Pro Ser Gln
1 5 10 15
Thr Leu Ser Leu Thr Cys Thr Val Ser Gly Phe Thr Ile Ser Ser Gly
20 25 30
Tyr Ser Trp His Trp Val Arg Gln Pro Pro Gly Arg Gly Leu Glu Trp
35 40 45
Ile Gly Tyr Ile Gln Tyr Ser Gly Ile Thr Asn Tyr Asn Pro Ser Leu
50 55 60
Lys Ser Arg Val Thr Met Leu Val Asp Thr Ser Lys Asn Gln Phe Ser
65 70 75 80
Leu Arg Leu Ser Ser Val Thr Ala Ala Asp Thr Ala Val Tyr Tyr Cys
85 90 95
Ala Arg Glu Asp Tyr Asp Tyr His Trp Tyr Phe Asp Val Trp Gly Gln
100 105 110
Gly Ser Leu Val Thr Val Ser Ser Ala Ser Thr Lys Gly Pro Ser Val
115 120 125
Phe Pro Leu Ala Pro Ser Ser Lys Ser Thr Ser Gly Gly Thr Ala Ala
130 135 140
Leu Gly Cys Leu Val Lys Asp Tyr Phe Pro Glu Pro Val Thr Val Ser
145 150 155 160
Trp Asn Ser Gly Ala Leu Thr Ser Gly Val His Thr Phe Pro Ala Val
165 170 175
Leu Gln Ser Ser Gly Leu Tyr Ser Leu Ser Ser Val Val Thr Val Pro
180 185 190
Ser Ser Ser Leu Gly Thr Gln Thr Tyr Ile Cys Asn Val Asn His Lys
195 200 205
Pro Ser Asn Thr Lys Val Asp Lys Lys Val Glu Pro Lys Ser Cys Gly
210 215 220
Gly Gly Gly Ser Gly Gly Gly Gly Ser Glu Val Gln Leu Val Glu Ser
225 230 235 240
Gly Gly Gly Leu Val Gln Pro Gly Gly Ser Leu Arg Leu Ser Cys Ala
245 250 255
Ala Ser Gly Tyr Ser Phe Thr Gly Tyr Thr Met Asn Trp Val Arg Gln
260 265 270
Ala Pro Gly Lys Gly Leu Glu Trp Val Ala Leu Ile Asn Pro Tyr Lys
275 280 285
Gly Val Ser Thr Tyr Asn Gln Lys Phe Lys Asp Arg Phe Thr Ile Ser
290 295 300
Val Asp Lys Ser Lys Asn Thr Ala Tyr Leu Gln Met Asn Ser Leu Arg
305 310 315 320
Ala Glu Asp Thr Ala Val Tyr Tyr Cys Ala Arg Ser Gly Tyr Tyr Gly
325 330 335
Asp Ser Asp Trp Tyr Phe Asp Val Trp Gly Gln Gly Thr Leu Val Thr
340 345 350
Val Ser Ser Ala Ser Val Ala Ala Pro Ser Val Phe Ile Phe Pro Pro
355 360 365
Ser Asp Glu Gln Leu Lys Ser Gly Thr Ala Ser Val Val Cys Leu Leu
370 375 380
Asn Asn Phe Tyr Pro Arg Glu Ala Lys Val Gln Trp Lys Val Asp Asn
385 390 395 400
Ala Leu Gln Ser Gly Asn Ser Gln Glu Ser Val Thr Glu Gln Asp Ser
405 410 415
Lys Asp Ser Thr Tyr Ser Leu Ser Ser Thr Leu Thr Leu Ser Lys Ala
420 425 430
Asp Tyr Glu Lys His Lys Val Tyr Ala Cys Glu Val Thr His Gln Gly
435 440 445
Leu Ser Ser Pro Val Thr Lys Ser Phe Asn Arg Gly Glu Cys Asp Lys
450 455 460
Thr His Thr Cys Pro Pro Cys Pro Ala Pro Glu Ala Ala Gly Gly Pro
465 470 475 480
Ser Val Phe Leu Phe Pro Pro Lys Pro Lys Asp Thr Leu Met Ile Ser
485 490 495
Arg Thr Pro Glu Val Thr Cys Val Val Val Asp Val Ser His Glu Asp
500 505 510
Pro Glu Val Lys Phe Asn Trp Tyr Val Asp Gly Val Glu Val His Asn
515 520 525
Ala Lys Thr Lys Pro Arg Glu Glu Gln Tyr Asn Ser Thr Tyr Arg Val
530 535 540
Val Ser Val Leu Thr Val Leu His Gln Asp Trp Leu Asn Gly Lys Glu
545 550 555 560
Tyr Lys Cys Lys Val Ser Asn Lys Ala Leu Gly Ala Pro Ile Glu Lys
565 570 575
Thr Ile Ser Lys Ala Lys Gly Gln Pro Arg Glu Pro Gln Val Tyr Thr
580 585 590
Leu Pro Pro Cys Arg Asp Glu Leu Thr Lys Asn Gln Val Ser Leu Trp
595 600 605
Cys Leu Val Lys Gly Phe Tyr Pro Ser Asp Ile Ala Val Glu Trp Glu
610 615 620
Ser Asn Gly Gln Pro Glu Asn Asn Tyr Lys Thr Thr Pro Pro Val Leu
625 630 635 640
Asp Ser Asp Gly Ser Phe Phe Leu Tyr Ser Lys Leu Thr Val Asp Lys
645 650 655
Ser Arg Trp Gln Gln Gly Asn Val Phe Ser Cys Ser Val Met His Glu
660 665 670
Ala Leu His Asn His Tyr Thr Gln Lys Ser Leu Ser Leu Ser Pro Gly
675 680 685
Lys

SEQ ID NO: 206
<212> DNA
<223> 431/26 (VH-CH1)-V9(VH-CL)-Fc(knob) P329G LALA
caggtgcagc tgcaggaatc tggccctgga ctcgtgcggc ctagccagac actgagcctg 60
acctgtaccg tgtccggctt caccatcagc agcggctaca gctggcattg ggtgcgccag 120
ccacctggca gaggcctgga atggatcggc tacatccagt acagcggcat caccaactac 180
aaccccagcc tgaagtccag agtgaccatg ctggtggaca cctccaagaa ccagttcagc 240
ctgcggctga gcagcgtgac agccgccgat acagccgtgt actactgcgc cagagaggac 300
tacgactacc actggtactt cgacgtgtgg ggccagggct ctctcgtgac cgtgtcaagc 360
gctagcacaa agggccccag cgtgttccct ctggccccta gcagcaagag cacatctggc 420
ggaacagccg ccctgggctg cctggtcaag gactactttc ccgagcccgt gacagtgtcc 480
tggaactctg gcgccctgac aagcggcgtg cacacctttc cagccgtgct gcagagcagc 540
ggcctgtact ctctgagcag cgtggtcacc gtgcctagct ctagcctggg cacccagacc 600
tacatctgca acgtgaacca caagcccagc aacaccaagg tggacaagaa ggtggaaccc 660
aagagctgcg gcggaggcgg atccggaggc ggaggatccg aagtgcagct ggtggaatct 720
ggcggaggcc tggtgcagcc tggcggatct ctgagactga gctgtgccgc cagcggctac 780
agcttcaccg gctacaccat gaactgggtg cgccaggccc ctggcaaggg actggaatgg 840
gtggccctga tcaaccccta caagggcgtg tccacataca accagaagtt caaggaccgg 900
ttcaccatca gcgtggacaa gagcaagaac accgcctacc tgcagatgaa cagcctgcgg 960
gccgaggaca ccgccgtgta ctattgtgcc agaagcggct actacggcga cagcgactgg 1020
tacttcgacg tgtggggcca gggcacactc gtgaccgtgt caagcgctag cgtggccgct 1080
ccctccgtgt ttatctttcc cccatccgat gaacagctga aaagcggcac cgcctccgtc 1140
gtgtgtctgc tgaacaattt ttaccctagg gaagctaaag tgcagtggaa agtggataac 1200
gcactgcagt ccggcaactc ccaggaatct gtgacagaac aggactccaa ggacagcacc 1260
tactccctgt cctccaccct gacactgtct aaggctgatt atgagaaaca caaagtctac 1320
gcctgcgaag tcacccatca gggcctgagc tcgcccgtca caaagagctt caacagggga 1380
gagtgtgaca agacccacac ctgtccccct tgtcctgccc ctgaagctgc tggcggccct 1440
tctgtgttcc tgttcccccc aaagcccaag gacaccctga tgatcagccg gacccccgaa 1500
gtgacctgcg tggtggtgga tgtgtcccac gaggaccctg aagtgaagtt caattggtac 1560
gtggacggcg tggaagtgca caacgccaag acaaagccgc gggaggagca gtacaacagc 1620
acgtaccgtg tggtcagcgt cctcaccgtc ctgcaccagg actggctgaa tggcaaggag 1680
tacaagtgca aggtctccaa caaagccctc ggcgccccca tcgagaaaac catctccaaa 1740
gccaaagggc agccccgaga accacaggtg tacaccctgc ccccatgccg ggatgagctg 1800
accaagaacc aggtcagcct gtggtgcctg gtcaaaggct tctatcccag cgacatcgcc 1860
gtggagtggg agagcaatgg gcagccggag aacaactaca agaccacgcc tcccgtgctg 1920
gactccgacg gctccttctt cctctacagc aagctcaccg tggacaagag caggtggcag 1980
caggggaacg tcttctcatg ctccgtgatg catgaggctc tgcacaacca ctacacgcag 2040
aagagcctct ccctgtctcc gggtaaa 2067

SEQ ID NO: 207
<212> PRT
<223> 431/26 (VH-CH1)-Fc(hole) P329G LALA
Gln Val Gln Leu Gln Glu Ser Gly Pro Gly Leu Val Arg Pro Ser Gln
1 5 10 15
Thr Leu Ser Leu Thr Cys Thr Val Ser Gly Phe Thr Ile Ser Ser Gly
20 25 30
Tyr Ser Trp His Trp Val Arg Gln Pro Pro Gly Arg Gly Leu Glu Trp
35 40 45
Ile Gly Tyr Ile Gln Tyr Ser Gly Ile Thr Asn Tyr Asn Pro Ser Leu
50 55 60
Lys Ser Arg Val Thr Met Leu Val Asp Thr Ser Lys Asn Gln Phe Ser
65 70 75 80
Leu Arg Leu Ser Ser Val Thr Ala Ala Asp Thr Ala Val Tyr Tyr Cys
85 90 95
Ala Arg Glu Asp Tyr Asp Tyr His Trp Tyr Phe Asp Val Trp Gly Gln
100 105 110
Gly Ser Leu Val Thr Val Ser Ser Ala Ser Thr Lys Gly Pro Ser Val
115 120 125
Phe Pro Leu Ala Pro Ser Ser Lys Ser Thr Ser Gly Gly Thr Ala Ala
130 135 140
Leu Gly Cys Leu Val Lys Asp Tyr Phe Pro Glu Pro Val Thr Val Ser
145 150 155 160
Trp Asn Ser Gly Ala Leu Thr Ser Gly Val His Thr Phe Pro Ala Val
165 170 175
Leu Gln Ser Ser Gly Leu Tyr Ser Leu Ser Ser Val Val Thr Val Pro
180 185 190
Ser Ser Ser Leu Gly Thr Gln Thr Tyr Ile Cys Asn Val Asn His Lys
195 200 205
Pro Ser Asn Thr Lys Val Asp Lys Lys Val Glu Pro Lys Ser Cys Asp
210 215 220
Lys Thr His Thr Cys Pro Pro Cys Pro Ala Pro Glu Ala Ala Gly Gly
225 230 235 240
Pro Ser Val Phe Leu Phe Pro Pro Lys Pro Lys Asp Thr Leu Met Ile
245 250 255
Ser Arg Thr Pro Glu Val Thr Cys Val Val Val Asp Val Ser His Glu
260 265 270
Asp Pro Glu Val Lys Phe Asn Trp Tyr Val Asp Gly Val Glu Val His
275 280 285
Asn Ala Lys Thr Lys Pro Arg Glu Glu Gln Tyr Asn Ser Thr Tyr Arg
290 295 300
Val Val Ser Val Leu Thr Val Leu His Gln Asp Trp Leu Asn Gly Lys
305 310 315 320
Glu Tyr Lys Cys Lys Val Ser Asn Lys Ala Leu Gly Ala Pro Ile Glu
325 330 335
Lys Thr Ile Ser Lys Ala Lys Gly Gln Pro Arg Glu Pro Gln Val Cys
340 345 350
Thr Leu Pro Pro Ser Arg Asp Glu Leu Thr Lys Asn Gln Val Ser Leu
355 360 365
Ser Cys Ala Val Lys Gly Phe Tyr Pro Ser Asp Ile Ala Val Glu Trp
370 375 380
Glu Ser Asn Gly Gln Pro Glu Asn Asn Tyr Lys Thr Thr Pro Pro Val
385 390 395 400
Leu Asp Ser Asp Gly Ser Phe Phe Leu Val Ser Lys Leu Thr Val Asp
405 410 415
Lys Ser Arg Trp Gln Gln Gly Asn Val Phe Ser Cys Ser Val Met His
420 425 430
Glu Ala Leu His Asn His Tyr Thr Gln Lys Ser Leu Ser Leu Ser Pro
435 440 445
Gly Lys
450

SEQ ID NO: 208
<212> DNA
<223> 431/26 (VH-CH1)-Fc(hole) P329G LALA
caggtgcagc tgcaggaatc tggccctgga ctcgtgcggc ctagccagac actgagcctg 60
acctgtaccg tgtccggctt caccatcagc agcggctaca gctggcattg ggtgcgccag 120
ccacctggca gaggcctgga atggatcggc tacatccagt acagcggcat caccaactac 180
aaccccagcc tgaagtccag agtgaccatg ctggtggaca cctccaagaa ccagttcagc 240
ctgcggctga gcagcgtgac agccgccgat acagccgtgt actactgcgc cagagaggac 300
tacgactacc actggtactt cgacgtgtgg ggccagggct ctctcgtgac cgtgtcaagc 360
gctagcacca agggcccctc cgtgttcccc ctggccccca gcagcaagag caccagcggc 420
ggcacagccg ctctgggctg cctggtcaag gactacttcc ccgagcccgt gaccgtgtcc 480
tggaacagcg gagccctgac ctccggcgtg cacaccttcc ccgccgtgct gcagagttct 540
ggcctgtata gcctgagcag cgtggtcacc gtgccttcta gcagcctggg cacccagacc 600
tacatctgca acgtgaacca caagcccagc aacaccaagg tggacaagaa ggtggagccc 660
aagagctgcg acaaaactca cacatgccca ccgtgcccag cacctgaagc tgcaggggga 720
ccgtcagtct tcctcttccc cccaaaaccc aaggacaccc tcatgatctc ccggacccct 780
gaggtcacat gcgtggtggt ggacgtgagc cacgaagacc ctgaggtcaa gttcaactgg 840
tacgtggacg gcgtggaggt gcataatgcc aagacaaagc cgcgggagga gcagtacaac 900
agcacgtacc gtgtggtcag cgtcctcacc gtcctgcacc aggactggct gaatggcaag 960
gagtacaagt gcaaggtctc caacaaagcc ctcggcgccc ccatcgagaa aaccatctcc 1020
aaagccaaag ggcagccccg agaaccacag gtgtgcaccc tgcccccatc ccgggatgag 1080
ctgaccaaga accaggtcag cctctcgtgc gcagtcaaag gcttctatcc cagcgacatc 1140
gccgtggagt gggagagcaa tgggcagccg gagaacaact acaagaccac gcctcccgtg 1200
ctggactccg acggctcctt cttcctcgtg agcaagctca ccgtggacaa gagcaggtgg 1260
cagcagggga acgtcttctc atgctccgtg atgcatgagg ctctgcacaa ccactacacg 1320
cagaagagcc tctccctgtc tccgggtaaa 1350

SEQ ID NO: 209
<212> PRT
<223> CH1A1A(VL-CL)-V9 (VH-CL)
Asp Ile Gln Met Thr Gln Ser Pro Ser Ser Leu Ser Ala Ser Val Gly
1 5 10 15
Asp Arg Val Thr Ile Thr Cys Lys Ala Ser Ala Ala Val Gly Thr Tyr
20 25 30
Val Ala Trp Tyr Gln Gln Lys Pro Gly Lys Ala Pro Lys Leu Leu Ile
35 40 45
Tyr Ser Ala Ser Tyr Arg Lys Arg Gly Val Pro Ser Arg Phe Ser Gly
50 55 60
Ser Gly Ser Gly Thr Asp Phe Thr Leu Thr Ile Ser Ser Leu Gln Pro
65 70 75 80
Glu Asp Phe Ala Thr Tyr Tyr Cys His Gln Tyr Tyr Thr Tyr Pro Leu
85 90 95
Phe Thr Phe Gly Gln Gly Thr Lys Leu Glu Ile Lys Arg Thr Val Ala
100 105 110
Ala Pro Ser Val Phe Ile Phe Pro Pro Ser Asp Glu Gln Leu Lys Ser
115 120 125
Gly Thr Ala Ser Val Val Cys Leu Leu Asn Asn Phe Tyr Pro Arg Glu
130 135 140
Ala Lys Val Gln Trp Lys Val Asp Asn Ala Leu Gln Ser Gly Asn Ser
145 150 155 160
Gln Glu Ser Val Thr Glu Gln Asp Ser Lys Asp Ser Thr Tyr Ser Leu
165 170 175
Ser Ser Thr Leu Thr Leu Ser Lys Ala Asp Tyr Glu Lys His Lys Val
180 185 190
Tyr Ala Cys Glu Val Thr His Gln Gly Leu Ser Ser Pro Val Thr Lys
195 200 205
Ser Phe Asn Arg Gly Glu Cys Gly Gly Gly Gly Ser Gly Gly Gly Gly
210 215 220
Ser Gly Gly Gly Gly Ser Gly Gly Gly Gly Ser Glu Val Gln Leu Val
225 230 235 240
Glu Ser Gly Gly Gly Leu Val Gln Pro Gly Gly Ser Leu Arg Leu Ser
245 250 255
Cys Ala Ala Ser Gly Tyr Ser Phe Thr Gly Tyr Thr Met Asn Trp Val
260 265 270
Arg Gln Ala Pro Gly Lys Gly Leu Glu Trp Val Ala Leu Ile Asn Pro
275 280 285
Tyr Lys Gly Val Ser Thr Tyr Asn Gln Lys Phe Lys Asp Arg Phe Thr
290 295 300
Ile Ser Val Asp Lys Ser Lys Asn Thr Ala Tyr Leu Gln Met Asn Ser
305 310 315 320
Leu Arg Ala Glu Asp Thr Ala Val Tyr Tyr Cys Ala Arg Ser Gly Tyr
325 330 335
Tyr Gly Asp Ser Asp Trp Tyr Phe Asp Val Trp Gly Gln Gly Thr Leu
340 345 350
Val Thr Val Ser Ser Ala Ser Val Ala Ala Pro Ser Val Phe Ile Phe
355 360 365
Pro Pro Ser Asp Glu Gln Leu Lys Ser Gly Thr Ala Ser Val Val Cys
370 375 380
Leu Leu Asn Asn Phe Tyr Pro Arg Glu Ala Lys Val Gln Trp Lys Val
385 390 395 400
Asp Asn Ala Leu Gln Ser Gly Asn Ser Gln Glu Ser Val Thr Glu Gln
405 410 415
Asp Ser Lys Asp Ser Thr Tyr Ser Leu Ser Ser Thr Leu Thr Leu Ser
420 425 430
Lys Ala Asp Tyr Glu Lys His Lys Val Tyr Ala Cys Glu Val Thr His
435 440 445
Gln Gly Leu Ser Ser Pro Val Thr Lys Ser Phe Asn Arg Gly Glu Cys
450 455 460

SEQ ID NO: 210
<212> DNA
<223> CH1A1A(VL-CL)-V9 (VH-CL)
gatatccaga tgacccagag ccccagcagc ctgtctgcca gcgtgggcga cagagtgacc 60
atcacatgca aggcctctgc cgccgtgggc acatacgtgg cctggtatca gcagaagccc 120
ggcaaggccc ccaagctgct gatctacagc gccagctacc ggaagagagg cgtgcccagc 180
agattttccg gcagcggctc tggcaccgac ttcaccctga ccatcagctc cctgcagccc 240
gaggacttcg ccacctacta ctgccaccag tactacacct accccctgtt caccttcggc 300
cagggcacca agctcgagat caagcgtacg gtggccgctc ccagcgtgtt catcttccca 360
cctagcgacg agcagctgaa gtccggcaca gcctctgtcg tgtgcctgct gaacaacttc 420
tacccccgcg aggccaaggt gcagtggaag gtggacaatg ccctgcagag cggcaacagc 480
caggaaagcg tgaccgagca ggacagcaag gactccacct acagcctgag cagcaccctg 540
acactgagca aggccgacta cgagaagcac aaggtgtacg cctgcgaagt gacccaccag 600
ggcctgtcta gccccgtgac caagagcttc aaccggggcg aatgtggcgg cggaggatcc 660
ggcggaggcg gctccggagg cggaggaagt ggcggagggg gatctgaagt gcagctggtg 720
gaatctggcg gaggcctggt gcagcctggc ggatctctga gactgagctg tgccgccagc 780
ggctacagct tcaccggcta caccatgaac tgggtgcgcc aggcccctgg caagggactg 840
gaatgggtgg ccctgatcaa cccctacaag ggcgtgtcca catacaacca gaagttcaag 900
gaccggttca ccatcagcgt ggacaagagc aagaacaccg cctacctgca gatgaacagc 960
ctgcgggccg aggacaccgc cgtgtactac tgtgccagaa gcggctacta cggcgacagc 1020
gactggtact tcgacgtgtg gggccaggga accctcgtga ccgtgtcaag cgctagcgtg 1080
gccgcaccct ctgtgtttat ctttccaccc tctgacgaac agctgaaaag cggcaccgcc 1140
agcgtcgtgt gtctgctgaa caatttttac cctagggaag ctaaagtgca gtggaaagtg 1200
gataacgcac tgcagtccgg caactcccag gaatctgtga cagaacagga ctccaaggac 1260
agcacatact ccctgtccag cacactgacc ctgtctaagg ccgattatga gaaacacaaa 1320
gtgtatgctt gtgaagtgac acatcaggga ctgagcagcc ctgtgacaaa gtccttcaac 1380
agaggcgagt gt 1392

SEQ ID NO: 211
<212> PRT
<223> CH1A1A(VH-CH1)-Fc(knob) P329G LALA
Gln Val Gln Leu Val Gln Ser Gly Ala Glu Val Lys Lys Pro Gly Ala
1 5 10 15
Ser Val Lys Val Ser Cys Lys Ala Ser Gly Tyr Thr Phe Thr Glu Phe
20 25 30
Gly Met Asn Trp Val Arg Gln Ala Pro Gly Gln Gly Leu Glu Trp Met
35 40 45
Gly Trp Ile Asn Thr Lys Thr Gly Glu Ala Thr Tyr Val Glu Glu Phe
50 55 60
Lys Gly Arg Val Thr Phe Thr Thr Asp Thr Ser Thr Ser Thr Ala Tyr
65 70 75 80
Met Glu Leu Arg Ser Leu Arg Ser Asp Asp Thr Ala Val Tyr Tyr Cys
85 90 95
Ala Arg Trp Asp Phe Ala Tyr Tyr Val Glu Ala Met Asp Tyr Trp Gly
100 105 110
Gln Gly Thr Thr Val Thr Val Ser Ser Ala Ser Thr Lys Gly Pro Ser
115 120 125
Val Phe Pro Leu Ala Pro Ser Ser Lys Ser Thr Ser Gly Gly Thr Ala
130 135 140
Ala Leu Gly Cys Leu Val Lys Asp Tyr Phe Pro Glu Pro Val Thr Val
145 150 155 160
Ser Trp Asn Ser Gly Ala Leu Thr Ser Gly Val His Thr Phe Pro Ala
165 170 175
Val Leu Gln Ser Ser Gly Leu Tyr Ser Leu Ser Ser Val Val Thr Val
180 185 190
Pro Ser Ser Ser Leu Gly Thr Gln Thr Tyr Ile Cys Asn Val Asn His
195 200 205
Lys Pro Ser Asn Thr Lys Val Asp Lys Lys Val Glu Pro Lys Ser Cys
210 215 220
Asp Lys Thr His Thr Cys Pro Pro Cys Pro Ala Pro Glu Ala Ala Gly
225 230 235 240
Gly Pro Ser Val Phe Leu Phe Pro Pro Lys Pro Lys Asp Thr Leu Met
245 250 255
Ile Ser Arg Thr Pro Glu Val Thr Cys Val Val Val Asp Val Ser His
260 265 270
Glu Asp Pro Glu Val Lys Phe Asn Trp Tyr Val Asp Gly Val Glu Val
275 280 285
His Asn Ala Lys Thr Lys Pro Arg Glu Glu Gln Tyr Asn Ser Thr Tyr
290 295 300
Arg Val Val Ser Val Leu Thr Val Leu His Gln Asp Trp Leu Asn Gly
305 310 315 320
Lys Glu Tyr Lys Cys Lys Val Ser Asn Lys Ala Leu Gly Ala Pro Ile
325 330 335
Glu Lys Thr Ile Ser Lys Ala Lys Gly Gln Pro Arg Glu Pro Gln Val
340 345 350
Tyr Thr Leu Pro Pro Cys Arg Asp Glu Leu Thr Lys Asn Gln Val Ser
355 360 365
Leu Trp Cys Leu Val Lys Gly Phe Tyr Pro Ser Asp Ile Ala Val Glu
370 375 380
Trp Glu Ser Asn Gly Gln Pro Glu Asn Asn Tyr Lys Thr Thr Pro Pro
385 390 395 400
Val Leu Asp Ser Asp Gly Ser Phe Phe Leu Tyr Ser Lys Leu Thr Val
405 410 415
Asp Lys Ser Arg Trp Gln Gln Gly Asn Val Phe Ser Cys Ser Val Met
420 425 430
His Glu Ala Leu His Asn His Tyr Thr Gln Lys Ser Leu Ser Leu Ser
435 440 445
Pro Gly Lys
450

SEQ ID NO: 212
<212> DNA
<223> CH1A1A(VH-CH1)-Fc(knob) P329G LALA
caggtgcagc tggtgcagtc tggcgccgaa gtgaagaaac ctggagctag tgtgaaggtg 60
tcctgcaagg ccagcggcta caccttcacc gagttcggca tgaactgggt ccgacaggct 120
ccaggccagg gcctcgaatg gatgggctgg atcaacacca agaccggcga ggccacctac 180
gtggaagagt tcaagggcag agtgaccttc accacggaca ccagcaccag caccgcctac 240
atggaactgc ggagcctgag aagcgacgac accgccgtgt actactgcgc cagatgggac 300
ttcgcctatt acgtggaagc catggactac tggggccagg gcaccaccgt gaccgtgtct 360
agcgctagca ccaagggccc atcggtcttc cccctggcac cctcctccaa gagcacctct 420
gggggcacag cggccctggg ctgcctggtc aaggactact tccccgaacc ggtgacggtg 480
tcgtggaact caggcgccct gaccagcggc gtgcacacct tcccggctgt cctacagtcc 540
tcaggactct actccctcag cagcgtggtg accgtgccct ccagcagctt gggcacccag 600
acctacatct gcaacgtgaa tcacaagccc agcaacacca aggtggacaa gaaagttgag 660
cccaaatctt gtgacaaaac tcacacatgc ccaccgtgcc cagcacctga agctgcaggg 720
ggaccgtcag tcttcctctt ccccccaaaa cccaaggaca ccctcatgat ctcccggacc 780
cctgaggtca catgcgtggt ggtggacgtg agccacgaag accctgaggt caagttcaac 840
tggtacgtgg acggcgtgga ggtgcataat gccaagacaa agccgcggga ggagcagtac 900
aacagcacgt accgtgtggt cagcgtcctc accgtcctgc accaggactg gctgaatggc 960
aaggagtaca agtgcaaggt ctccaacaaa gccctcggcg cccccatcga gaaaaccatc 1020
tccaaagcca aagggcagcc ccgagaacca caggtgtaca ccctgccccc atgccgggat 1080
gagctgacca agaaccaggt cagcctgtgg tgcctggtca aaggcttcta tcccagcgac 1140
atcgccgtgg agtgggagag caatgggcag ccggagaaca actacaagac cacgcctccc 1200
gtgctggact ccgacggctc cttcttcctc tacagcaagc tcaccgtgga caagagcagg 1260
tggcagcagg ggaacgtctt ctcatgctcc gtgatgcatg aggctctgca caaccactac 1320
acgcagaaga gcctctccct gtctccgggt aaa 1353

SEQ ID NO: 213
<212> PRT
<223> Fc(hole) P329G LALA
Asp Lys Thr His Thr Cys Pro Pro Cys Pro Ala Pro Glu Ala Ala Gly
1 5 10 15
Gly Pro Ser Val Phe Leu Phe Pro Pro Lys Pro Lys Asp Thr Leu Met
20 25 30
Ile Ser Arg Thr Pro Glu Val Thr Cys Val Val Val Asp Val Ser His
35 40 45
Glu Asp Pro Glu Val Lys Phe Asn Trp Tyr Val Asp Gly Val Glu Val
50 55 60
His Asn Ala Lys Thr Lys Pro Arg Glu Glu Gln Tyr Asn Ser Thr Tyr
65 70 75 80
Arg Val Val Ser Val Leu Thr Val Leu His Gln Asp Trp Leu Asn Gly
85 90 95
Lys Glu Tyr Lys Cys Lys Val Ser Asn Lys Ala Leu Gly Ala Pro Ile
100 105 110
Glu Lys Thr Ile Ser Lys Ala Lys Gly Gln Pro Arg Glu Pro Gln Val
115 120 125
Cys Thr Leu Pro Pro Ser Arg Asp Glu Leu Thr Lys Asn Gln Val Ser
130 135 140
Leu Ser Cys Ala Val Lys Gly Phe Tyr Pro Ser Asp Ile Ala Val Glu
145 150 155 160
Trp Glu Ser Asn Gly Gln Pro Glu Asn Asn Tyr Lys Thr Thr Pro Pro
165 170 175
Val Leu Asp Ser Asp Gly Ser Phe Phe Leu Val Ser Lys Leu Thr Val
180 185 190
Asp Lys Ser Arg Trp Gln Gln Gly Asn Val Phe Ser Cys Ser Val Met
195 200 205
His Glu Ala Leu His Asn His Tyr Thr Gln Lys Ser Leu Ser Leu Ser
210 215 220
Pro Gly Lys
225

SEQ ID NO: 214
<212> DNA
<223> Fc(hole) P329G LALA
gacaaaactc acacatgccc accgtgccca gcacctgaag ctgcaggggg accgtcagtc 60
ttcctcttcc ccccaaaacc caaggacacc ctcatgatct cccggacccc tgaggtcaca 120
tgcgtggtgg tggacgtgag ccacgaagac cctgaggtca agttcaactg gtacgtggac 180
ggcgtggagg tgcataatgc caagacaaag ccgcgggagg agcagtacaa cagcacgtac 240
cgtgtggtca gcgtcctcac cgtcctgcac caggactggc tgaatggcaa ggagtacaag 300
tgcaaggtct ccaacaaagc cctcggcgcc cccatcgaga aaaccatctc caaagccaaa 360
gggcagcccc gagaaccaca ggtgtgcacc ctgcccccat cccgggatga gctgaccaag 420
aaccaggtca gcctctcgtg cgcagtcaaa ggcttctatc ccagcgacat cgccgtggag 480
tgggagagca atgggcagcc ggagaacaac tacaagacca cgcctcccgt gctggactcc 540
gacggctcct tcttcctcgt gagcaagctc accgtggaca agagcaggtg gcagcagggg 600
aacgtcttct catgctccgt gatgcatgag gctctgcaca accactacac gcagaagagc 660
ctctccctgt ctccgggtaa a 681

SEQ ID NO: 215
<212> PRT
<223> CH2527 (VL-CH1)
Gln Ala Val Val Thr Gln Glu Ser Ala Leu Thr Thr Ser Pro Gly Glu
1 5 10 15
Thr Val Thr Leu Thr Cys Arg Ser Ser Thr Gly Ala Val Thr Thr Ser
20 25 30
Asn Tyr Ala Asn Trp Val Gln Glu Lys Pro Asp His Leu Phe Thr Gly
35 40 45
Leu Ile Gly Gly Thr Asn Lys Arg Ala Pro Gly Val Pro Ala Arg Phe
50 55 60
Ser Gly Ser Leu Ile Gly Asp Lys Ala Ala Leu Thr Ile Thr Gly Ala
65 70 75 80
Gln Thr Glu Asp Glu Ala Ile Tyr Phe Cys Ala Leu Trp Tyr Ser Asn
85 90 95
Leu Trp Val Phe Gly Gly Gly Thr Lys Leu Thr Val Leu Ser Ser Ala
100 105 110
Ser Thr Lys Gly Pro Ser Val Phe Pro Leu Ala Pro Ser Ser Lys Ser
115 120 125
Thr Ser Gly Gly Thr Ala Ala Leu Gly Cys Leu Val Lys Asp Tyr Phe
130 135 140
Pro Glu Pro Val Thr Val Ser Trp Asn Ser Gly Ala Leu Thr Ser Gly
145 150 155 160
Val His Thr Phe Pro Ala Val Leu Gln Ser Ser Gly Leu Tyr Ser Leu
165 170 175
Ser Ser Val Val Thr Val Pro Ser Ser Ser Leu Gly Thr Gln Thr Tyr
180 185 190
Ile Cys Asn Val Asn His Lys Pro Ser Asn Thr Lys Val Asp Lys Lys
195 200 205
Val Glu Pro Lys Ser Cys
210

SEQ ID NO: 216
<212> DNA
<223> CH2527 (VL-CH1)
caggccgtcg tgacccagga aagcgccctg acaacaagcc ctggcgagac agtgaccctg 60
acctgcagat ctagcacagg cgccgtgacc accagcaact acgccaactg ggtgcaggaa 120
aagcccgacc acctgttcac cggcctgatc ggcggcacca acaaaagggc tccaggcgtg 180
ccagccagat tcagcggcag cctgattggc gataaggccg ccctgaccat cactggcgcc 240
cagacagagg acgaggccat ctacttttgc gccctgtggt acagcaacct gtgggtgttc 300
ggcggaggca ccaagctgac agtgctgagc agcgcttcca ccaaaggccc ttccgtgttt 360
cctctggctc ctagctccaa gtccacctct ggaggcaccg ctgctctcgg atgcctcgtg 420
aaggattatt ttcctgagcc tgtgacagtg tcctggaata gcggagcact gacctctgga 480
gtgcatactt tccccgctgt gctgcagtcc tctggactgt acagcctgag cagcgtggtg 540
acagtgccca gcagcagcct gggcacccag acctacatct gcaacgtgaa ccacaagccc 600
agcaacacca aggtggacaa gaaggtggaa cccaagtctt gt 642

SEQ ID NO: 217
<212> PRT
<223> CH2527 (VH-CL)-LC007(VH-CH1)-Fc(knob) P329G LALA
Glu Val Gln Leu Val Glu Ser Gly Gly Gly Leu Val Gln Pro Lys Gly
1 5 10 15
Ser Leu Lys Leu Ser Cys Ala Ala Ser Gly Phe Thr Phe Asn Thr Tyr
20 25 30
Ala Met Asn Trp Val Arg Gln Ala Pro Gly Lys Gly Leu Glu Trp Val
35 40 45
Ala Arg Ile Arg Ser Lys Tyr Asn Asn Tyr Ala Thr Tyr Tyr Ala Asp
50 55 60
Ser Val Lys Asp Arg Phe Thr Ile Ser Arg Asp Asp Ser Gln Ser Ile
65 70 75 80
Leu Tyr Leu Gln Met Asn Asn Leu Lys Thr Glu Asp Thr Ala Met Tyr
85 90 95
Tyr Cys Val Arg His Gly Asn Phe Gly Asn Ser Tyr Val Ser Trp Phe
100 105 110
Ala Tyr Trp Gly Gln Gly Thr Leu Val Thr Val Ser Ala Ala Ser Val
115 120 125
Ala Ala Pro Ser Val Phe Ile Phe Pro Pro Ser Asp Glu Gln Leu Lys
130 135 140
Ser Gly Thr Ala Ser Val Val Cys Leu Leu Asn Asn Phe Tyr Pro Arg
145 150 155 160
Glu Ala Lys Val Gln Trp Lys Val Asp Asn Ala Leu Gln Ser Gly Asn
165 170 175
Ser Gln Glu Ser Val Thr Glu Gln Asp Ser Lys Asp Ser Thr Tyr Ser
180 185 190
Leu Ser Ser Thr Leu Thr Leu Ser Lys Ala Asp Tyr Glu Lys His Lys
195 200 205
Val Tyr Ala Cys Glu Val Thr His Gln Gly Leu Ser Ser Pro Val Thr
210 215 220
Lys Ser Phe Asn Arg Gly Glu Cys Gly Gly Gly Gly Ser Gly Gly Gly
225 230 235 240
Gly Ser Glu Val Gln Leu Gln Glu Ser Gly Pro Gly Leu Val Lys Pro
245 250 255
Ser Gln Ser Leu Ser Leu Thr Cys Ser Val Thr Gly Tyr Ser Ile Thr
260 265 270
Ser Gly Tyr Tyr Trp Asn Trp Ile Arg Gln Phe Pro Gly Asn Lys Leu
275 280 285
Glu Trp Met Gly Tyr Ile Thr Tyr Asp Gly Ser Asn Asn Tyr Asn Pro
290 295 300
Ser Leu Lys Asn Arg Ile Ser Ile Thr Arg Asp Thr Ser Lys Asn Gln
305 310 315 320
Phe Phe Leu Lys Leu Asn Ser Val Thr Thr Glu Asp Thr Ala Thr Tyr
325 330 335
Tyr Cys Ala Asp Phe Asp Tyr Trp Gly Gln Gly Thr Thr Leu Thr Val
340 345 350
Ser Ser Ala Ser Thr Lys Gly Pro Ser Val Phe Pro Leu Ala Pro Ser
355 360 365
Ser Lys Ser Thr Ser Gly Gly Thr Ala Ala Leu Gly Cys Leu Val Lys
370 375 380
Asp Tyr Phe Pro Glu Pro Val Thr Val Ser Trp Asn Ser Gly Ala Leu
385 390 395 400
Thr Ser Gly Val His Thr Phe Pro Ala Val Leu Gln Ser Ser Gly Leu
405 410 415
Tyr Ser Leu Ser Ser Val Val Thr Val Pro Ser Ser Ser Leu Gly Thr
420 425 430
Gln Thr Tyr Ile Cys Asn Val Asn His Lys Pro Ser Asn Thr Lys Val
435 440 445
Asp Lys Lys Val Glu Pro Lys Ser Cys Asp Lys Thr His Thr Cys Pro
450 455 460
Pro Cys Pro Ala Pro Glu Ala Ala Gly Gly Pro Ser Val Phe Leu Phe
465 470 475 480
Pro Pro Lys Pro Lys Asp Thr Leu Met Ile Ser Arg Thr Pro Glu Val
485 490 495
Thr Cys Val Val Val Asp Val Ser His Glu Asp Pro Glu Val Lys Phe
500 505 510
Asn Trp Tyr Val Asp Gly Val Glu Val His Asn Ala Lys Thr Lys Pro
515 520 525
Arg Glu Glu Gln Tyr Asn Ser Thr Tyr Arg Val Val Ser Val Leu Thr
530 535 540
Val Leu His Gln Asp Trp Leu Asn Gly Lys Glu Tyr Lys Cys Lys Val
545 550 555 560
Ser Asn Lys Ala Leu Gly Ala Pro Ile Glu Lys Thr Ile Ser Lys Ala
565 570 575
Lys Gly Gln Pro Arg Glu Pro Gln Val Tyr Thr Leu Pro Pro Cys Arg
580 585 590
Asp Glu Leu Thr Lys Asn Gln Val Ser Leu Trp Cys Leu Val Lys Gly
595 600 605
Phe Tyr Pro Ser Asp Ile Ala Val Glu Trp Glu Ser Asn Gly Gln Pro
610 615 620
Glu Asn Asn Tyr Lys Thr Thr Pro Pro Val Leu Asp Ser Asp Gly Ser
625 630 635 640
Phe Phe Leu Tyr Ser Lys Leu Thr Val Asp Lys Ser Arg Trp Gln Gln
645 650 655
Gly Asn Val Phe Ser Cys Ser Val Met His Glu Ala Leu His Asn His
660 665 670
Tyr Thr Gln Lys Ser Leu Ser Leu Ser Pro Gly Lys
675 680

SEQ ID NO: 218
<212> DNA
<223> CH2527 (VH-CL)-LC007(VH-CH1)-Fc(knob) P329G LALA
gaagtgcagc tggtggaaag cggcggaggc ctggtgcagc ctaagggctc tctgaagctg 60
agctgtgccg ccagcggctt caccttcaac acctacgcca tgaactgggt gcgccaggcc 120
cctggcaaag gcctggaatg ggtggcccgg atcagaagca agtacaacaa ttacgccacc 180
tactacgccg acagcgtgaa ggaccggttc accatcagcc gggacgacag ccagagcatc 240
ctgtacctgc agatgaacaa cctgaaaacc gaggacaccg ccatgtacta ctgcgtgcgg 300
cacggcaact tcggcaacag ctatgtgtct tggtttgcct actggggcca gggcaccctc 360
gtgacagtgt ctgctgctag cgtggctgca ccatctgtct tcatcttccc gccatctgat 420
gagcagttga aatctggaac tgcctctgtt gtgtgcctgc tgaataactt ctatcccaga 480
gaggccaaag tacagtggaa ggtggataac gccctccaat cgggtaactc ccaggagagt 540
gtcacagagc aggacagcaa ggacagcacc tacagcctca gcagcaccct gacgctgagc 600
aaagcagact acgagaaaca caaagtctac gcctgcgaag tcacccatca gggcctgagc 660
tcgcccgtca caaagagctt caacagggga gagtgtggcg gaggcggatc cggcggaggg 720
ggatctgagg tccagctgca ggagtcagga cctggcctcg tgaaaccttc tcagtctctg 780
tctctcacct gctctgtcac tggctactcc atcaccagtg gttattactg gaactggatt 840
cggcagtttc caggaaacaa gctggaatgg atgggctaca taacctacga cggtagcaat 900
aactacaacc catctctcaa aaatcgaatc tccattactc gtgacacatc taagaaccag 960
tttttcctga agttgaattc tgtgactact gaggacacag ctacatatta ctgtgcggac 1020
tttgactact ggggccaagg caccactctc acagtctcca gcgctagcac caagggcccc 1080
agcgtgttcc ccctggcacc cagcagcaag agcacatctg gcggaacagc cgctctgggc 1140
tgtctggtga aagactactt ccccgagccc gtgaccgtgt cttggaactc tggcgccctg 1200
accagcggcg tgcacacctt tccagccgtg ctgcagagca gcggcctgta ctccctgtcc 1260
tccgtggtca ccgtgccctc tagctccctg ggaacacaga catatatctg taatgtcaat 1320
cacaagcctt ccaacaccaa agtcgataag aaagtcgagc ccaagagctg cgacaaaact 1380
cacacatgcc caccgtgccc agcacctgaa gctgcagggg gaccgtcagt cttcctcttc 1440
cccccaaaac ccaaggacac cctcatgatc tcccggaccc ctgaggtcac atgcgtggtg 1500
gtggacgtga gccacgaaga ccctgaggtc aagttcaact ggtacgtgga cggcgtggag 1560
gtgcataatg ccaagacaaa gccgcgggag gagcagtaca acagcacgta ccgtgtggtc 1620
agcgtcctca ccgtcctgca ccaggactgg ctgaatggca aggagtacaa gtgcaaggtc 1680
tccaacaaag ccctcggcgc ccccatcgag aaaaccatct ccaaagccaa agggcagccc 1740
cgagaaccac aggtgtacac cctgccccca tgccgggatg agctgaccaa gaaccaggtc 1800
agcctgtggt gcctggtcaa aggcttctat cccagcgaca tcgccgtgga gtgggagagc 1860
aatgggcagc cggagaacaa ctacaagacc acgcctcccg tgctggactc cgacggctcc 1920
ttcttcctct acagcaagct caccgtggac aagagcaggt ggcagcaggg gaacgtcttc 1980
tcatgctccg tgatgcatga ggctctgcac aaccactaca cgcagaagag cctctccctg 2040
tctccgggta aa 2052

SEQ ID NO: 219
<212> PRT
<223> LC007(VH-CH1)-CH2527 (VH-CL)-Fc(knob) P329G LALA
Glu Val Gln Leu Gln Glu Ser Gly Pro Gly Leu Val Lys Pro Ser Gln
1 5 10 15
Ser Leu Ser Leu Thr Cys Ser Val Thr Gly Tyr Ser Ile Thr Ser Gly
20 25 30
Tyr Tyr Trp Asn Trp Ile Arg Gln Phe Pro Gly Asn Lys Leu Glu Trp
35 40 45
Met Gly Tyr Ile Thr Tyr Asp Gly Ser Asn Asn Tyr Asn Pro Ser Leu
50 55 60
Lys Asn Arg Ile Ser Ile Thr Arg Asp Thr Ser Lys Asn Gln Phe Phe
65 70 75 80
Leu Lys Leu Asn Ser Val Thr Thr Glu Asp Thr Ala Thr Tyr Tyr Cys
85 90 95
Ala Asp Phe Asp Tyr Trp Gly Gln Gly Thr Thr Leu Thr Val Ser Ser
100 105 110
Ala Ser Thr Lys Gly Pro Ser Val Phe Pro Leu Ala Pro Ser Ser Lys
115 120 125
Ser Thr Ser Gly Gly Thr Ala Ala Leu Gly Cys Leu Val Lys Asp Tyr
130 135 140
Phe Pro Glu Pro Val Thr Val Ser Trp Asn Ser Gly Ala Leu Thr Ser
145 150 155 160
Gly Val His Thr Phe Pro Ala Val Leu Gln Ser Ser Gly Leu Tyr Ser
165 170 175
Leu Ser Ser Val Val Thr Val Pro Ser Ser Ser Leu Gly Thr Gln Thr
180 185 190
Tyr Ile Cys Asn Val Asn His Lys Pro Ser Asn Thr Lys Val Asp Lys
195 200 205
Lys Val Glu Pro Lys Ser Cys Asp Gly Gly Gly Gly Ser Gly Gly Gly
210 215 220
Gly Ser Glu Val Gln Leu Val Glu Ser Gly Gly Gly Leu Val Gln Pro
225 230 235 240
Lys Gly Ser Leu Lys Leu Ser Cys Ala Ala Ser Gly Phe Thr Phe Asn
245 250 255
Thr Tyr Ala Met Asn Trp Val Arg Gln Ala Pro Gly Lys Gly Leu Glu
260 265 270
Trp Val Ala Arg Ile Arg Ser Lys Tyr Asn Asn Tyr Ala Thr Tyr Tyr
275 280 285
Ala Asp Ser Val Lys Asp Arg Phe Thr Ile Ser Arg Asp Asp Ser Gln
290 295 300
Ser Ile Leu Tyr Leu Gln Met Asn Asn Leu Lys Thr Glu Asp Thr Ala
305 310 315 320
Met Tyr Tyr Cys Val Arg His Gly Asn Phe Gly Asn Ser Tyr Val Ser
325 330 335
Trp Phe Ala Tyr Trp Gly Gln Gly Thr Leu Val Thr Val Ser Ala Ala
340 345 350
Ser Val Ala Ala Pro Ser Val Phe Ile Phe Pro Pro Ser Asp Glu Gln
355 360 365
Leu Lys Ser Gly Thr Ala Ser Val Val Cys Leu Leu Asn Asn Phe Tyr
370 375 380
Pro Arg Glu Ala Lys Val Gln Trp Lys Val Asp Asn Ala Leu Gln Ser
385 390 395 400
Gly Asn Ser Gln Glu Ser Val Thr Glu Gln Asp Ser Lys Asp Ser Thr
405 410 415
Tyr Ser Leu Ser Ser Thr Leu Thr Leu Ser Lys Ala Asp Tyr Glu Lys
420 425 430
His Lys Val Tyr Ala Cys Glu Val Thr His Gln Gly Leu Ser Ser Pro
435 440 445
Val Thr Lys Ser Phe Asn Arg Gly Glu Cys Asp Lys Thr His Thr Cys
450 455 460
Pro Pro Cys Pro Ala Pro Glu Ala Ala Gly Gly Pro Ser Val Phe Leu
465 470 475 480
Phe Pro Pro Lys Pro Lys Asp Thr Leu Met Ile Ser Arg Thr Pro Glu
485 490 495
Val Thr Cys Val Val Val Asp Val Ser His Glu Asp Pro Glu Val Lys
500 505 510
Phe Asn Trp Tyr Val Asp Gly Val Glu Val His Asn Ala Lys Thr Lys
515 520 525
Pro Arg Glu Glu Gln Tyr Asn Ser Thr Tyr Arg Val Val Ser Val Leu
530 535 540
Thr Val Leu His Gln Asp Trp Leu Asn Gly Lys Glu Tyr Lys Cys Lys
545 550 555 560
Val Ser Asn Lys Ala Leu Gly Ala Pro Ile Glu Lys Thr Ile Ser Lys
565 570 575
Ala Lys Gly Gln Pro Arg Glu Pro Gln Val Tyr Thr Leu Pro Pro Cys
580 585 590
Arg Asp Glu Leu Thr Lys Asn Gln Val Ser Leu Trp Cys Leu Val Lys
595 600 605
Gly Phe Tyr Pro Ser Asp Ile Ala Val Glu Trp Glu Ser Asn Gly Gln
610 615 620
Pro Glu Asn Asn Tyr Lys Thr Thr Pro Pro Val Leu Asp Ser Asp Gly
625 630 635 640
Ser Phe Phe Leu Tyr Ser Lys Leu Thr Val Asp Lys Ser Arg Trp Gln
645 650 655
Gln Gly Asn Val Phe Ser Cys Ser Val Met His Glu Ala Leu His Asn
660 665 670
His Tyr Thr Gln Lys Ser Leu Ser Leu Ser Pro Gly Lys
675 680 685

SEQ ID NO: 220
<212> DNA
<223> LC007(VH-CH1)-CH2527 (VH-CL)-Fc(knob) P329G LALA
gaggtgcagc tgcaggaatc tggccctggc ctggtcaagc caagccagag tctgagcctg 60
acctgcagcg tgaccggcta cagcattacc agcggctact actggaactg gattcggcag 120
ttccccggca ataagctgga atggatgggc tacatcacct acgacggcag caacaactac 180
aaccccagcc tgaagaaccg gatcagcatc acccgggaca ccagcaagaa ccagttcttc 240
ctgaagctga acagcgtgac caccgaggac accgccacat actattgcgc cgacttcgac 300
tactggggcc agggcaccac cctgaccgtg tccagcgcca gcacaaaggg ccctagcgtg 360
ttccctctgg cccccagcag caagagcaca agcggcggaa cagccgccct gggctgcctc 420
gtgaaggact acttccccga gcccgtgaca gtgtcttgga acagcggagc cctgacaagc 480
ggcgtgcaca ccttccctgc cgtgctgcag agcagcggcc tgtactccct gagcagcgtg 540
gtcaccgtgc ctagcagcag cctgggcacc cagacctaca tctgcaacgt gaaccacaag 600
cccagcaaca ccaaagtgga caagaaggtg gagcccaaga gctgtgatgg cggaggaggg 660
tccggaggcg gaggatccga agtgcagctg gtggaaagcg gcggaggcct ggtgcagcct 720
aagggctctc tgaagctgag ctgtgccgcc agcggcttca ccttcaacac ctacgccatg 780
aactgggtgc gccaggcccc tggcaaaggc ctggaatggg tggcccggat cagaagcaag 840
tacaacaatt acgccaccta ctacgccgac agcgtgaagg accggttcac catcagccgg 900
gacgacagcc agagcatcct gtacctgcag atgaacaacc tgaaaaccga ggacaccgcc 960
atgtactact gcgtgcggca cggcaacttc ggcaacagct atgtgtcttg gtttgcctac 1020
tggggccagg gcaccctcgt gacagtgtct gctgctagcg tggccgctcc ctccgtgttt 1080
atctttcccc catccgatga acagctgaaa agcggcaccg cctccgtcgt gtgtctgctg 1140
aacaattttt accctaggga agctaaagtg cagtggaaag tggataacgc actgcagtcc 1200
ggcaactccc aggaatctgt gacagaacag gactccaagg acagcaccta ctccctgtcc 1260
tccaccctga cactgtctaa ggctgattat gagaaacaca aagtctacgc ctgcgaagtc 1320
acccatcagg gcctgagctc gcccgtcaca aagagcttca acaggggaga gtgtgacaag 1380
acccacacct gtcccccttg tcctgcccct gaagctgctg gcggcccttc tgtgttcctg 1440
ttccccccaa agcccaagga caccctgatg atcagccgga cccccgaagt gacctgcgtg 1500
gtggtggatg tgtcccacga ggaccctgaa gtgaagttca attggtacgt ggacggcgtg 1560
gaagtgcaca acgccaagac aaagccgcgg gaggagcagt acaacagcac gtaccgtgtg 1620
gtcagcgtcc tcaccgtcct gcaccaggac tggctgaatg gcaaggagta caagtgcaag 1680
gtctccaaca aagccctcgg cgcccccatc gagaaaacca tctccaaagc caaagggcag 1740
ccccgagaac cacaggtgta caccctgccc ccatgccggg atgagctgac caagaaccag 1800
gtcagcctgt ggtgcctggt caaaggcttc tatcccagcg acatcgccgt ggagtgggag 1860
agcaatgggc agccggagaa caactacaag accacgcctc ccgtgctgga ctccgacggc 1920
tccttcttcc tctacagcaa gctcaccgtg gacaagagca ggtggcagca ggggaacgtc 1980
ttctcatgct ccgtgatgca tgaggctctg cacaaccact acacgcagaa gagcctctcc 2040
ctgtctccgg gtaaa 2055

SEQ ID NO: 221
<212> PRT
<223> anti-CD33(VL-CL)
Asp Ile Gln Met Thr Gln Ser Pro Ser Ser Leu Ser Ala Ser Val Gly
1 5 10 15
Asp Arg Val Thr Ile Thr Cys Arg Ala Ser Glu Ser Val Asp Asn Tyr
20 25 30
Gly Ile Ser Phe Met Asn Trp Phe Gln Gln Lys Pro Gly Lys Ala Pro
35 40 45
Lys Leu Leu Ile Tyr Ala Ala Ser Asn Gln Gly Ser Gly Val Pro Ser
50 55 60
Arg Phe Ser Gly Ser Gly Ser Gly Thr Asp Phe Thr Leu Thr Ile Ser
65 70 75 80
Ser Leu Gln Pro Asp Asp Phe Ala Thr Tyr Tyr Cys Gln Gln Ser Lys
85 90 95
Glu Val Pro Trp Thr Phe Gly Gln Gly Thr Lys Val Glu Ile Lys Arg
100 105 110
Thr Val Ala Ala Pro Ser Val Phe Ile Phe Pro Pro Ser Asp Glu Gln
115 120 125
Leu Lys Ser Gly Thr Ala Ser Val Val Cys Leu Leu Asn Asn Phe Tyr
130 135 140
Pro Arg Glu Ala Lys Val Gln Trp Lys Val Asp Asn Ala Leu Gln Ser
145 150 155 160
Gly Asn Ser Gln Glu Ser Val Thr Glu Gln Asp Ser Lys Asp Ser Thr
165 170 175
Tyr Ser Leu Ser Ser Thr Leu Thr Leu Ser Lys Ala Asp Tyr Glu Lys
180 185 190
His Lys Val Tyr Ala Cys Glu Val Thr His Gln Gly Leu Ser Ser Pro
195 200 205
Val Thr Lys Ser Phe Asn Arg Gly Glu Cys
210 215

SEQ ID NO: 222
<212> DNA
<223> anti-CD33(VL-CL)
gacatccaga tgacccagag ccccagcagc ctgagcgcca gcgtgggcga cagagtgacc 60
atcacctgtc gggccagcga gagcgtggac aactacggca tcagcttcat gaactggttc 120
cagcagaagc ccggcaaggc ccccaagctg ctgatctacg ccgccagcaa tcagggcagc 180
ggcgtgccca gcagattcag cggctctggc agcggcaccg acttcaccct gaccatcagc 240
agcctgcagc ccgacgactt cgccacctac tactgccagc agagcaaaga ggtgccctgg 300
accttcggcc agggcaccaa ggtggaaatc aagcgtacgg tggctgcacc atctgtcttc 360
atcttcccgc catctgatga gcagttgaaa tctggaactg cctctgttgt gtgcctgctg 420
aataacttct atcccagaga ggccaaagta cagtggaagg tggataacgc cctccaatcg 480
ggtaactccc aggagagtgt cacagagcag gacagcaagg acagcaccta cagcctcagc 540
agcaccctga cgctgagcaa agcagactac gagaaacaca aagtctacgc ctgcgaagtc 600
acccatcagg gcctgagctc gcccgtcaca aagagcttca acaggggaga gtgt 654

SEQ ID NO: 223
<212> PRT
<223> V9(VL-CH1)-anti-CD33(VH-CH1)-Fc(knob) P329G LALA
Asp Ile Gln Met Thr Gln Ser Pro Ser Ser Leu Ser Ala Ser Val Gly
1 5 10 15
Asp Arg Val Thr Ile Thr Cys Arg Ala Ser Gln Asp Ile Arg Asn Tyr
20 25 30
Leu Asn Trp Tyr Gln Gln Lys Pro Gly Lys Ala Pro Lys Leu Leu Ile
35 40 45
Tyr Tyr Thr Ser Arg Leu Glu Ser Gly Val Pro Ser Arg Phe Ser Gly
50 55 60
Ser Gly Ser Gly Thr Asp Tyr Thr Leu Thr Ile Ser Ser Leu Gln Pro
65 70 75 80
Glu Asp Phe Ala Thr Tyr Tyr Cys Gln Gln Gly Asn Thr Leu Pro Trp
85 90 95
Thr Phe Gly Gln Gly Thr Lys Val Glu Ile Lys Ser Ser Ala Ser Thr
100 105 110
Lys Gly Pro Ser Val Phe Pro Leu Ala Pro Ser Ser Lys Ser Thr Ser
115 120 125
Gly Gly Thr Ala Ala Leu Gly Cys Leu Val Lys Asp Tyr Phe Pro Glu
130 135 140
Pro Val Thr Val Ser Trp Asn Ser Gly Ala Leu Thr Ser Gly Val His
145 150 155 160
Thr Phe Pro Ala Val Leu Gln Ser Ser Gly Leu Tyr Ser Leu Ser Ser
165 170 175
Val Val Thr Val Pro Ser Ser Ser Leu Gly Thr Gln Thr Tyr Ile Cys
180 185 190
Asn Val Asn His Lys Pro Ser Asn Thr Lys Val Asp Lys Lys Val Glu
195 200 205
Pro Lys Ser Cys Gly Gly Gly Gly Ser Gly Gly Gly Gly Ser Gln Val
210 215 220
Gln Leu Val Gln Ser Gly Ala Glu Val Lys Lys Pro Gly Ser Ser Val
225 230 235 240
Lys Val Ser Cys Lys Ala Ser Gly Tyr Thr Phe Thr Asp Tyr Asn Met
245 250 255
His Trp Val Arg Gln Ala Pro Gly Gln Gly Leu Glu Trp Ile Gly Tyr
260 265 270
Ile Tyr Pro Tyr Asn Gly Gly Thr Gly Tyr Asn Gln Lys Phe Lys Ser
275 280 285
Lys Ala Thr Ile Thr Ala Asp Glu Ser Thr Asn Thr Ala Tyr Met Glu
290 295 300
Leu Ser Ser Leu Arg Ser Glu Asp Thr Ala Val Tyr Tyr Cys Ala Arg
305 310 315 320
Gly Arg Pro Ala Met Asp Tyr Trp Gly Gln Gly Thr Leu Val Thr Val
325 330 335
Ser Ser Ala Ser Thr Lys Gly Pro Ser Val Phe Pro Leu Ala Pro Ser
340 345 350
Ser Lys Ser Thr Ser Gly Gly Thr Ala Ala Leu Gly Cys Leu Val Lys
355 360 365
Asp Tyr Phe Pro Glu Pro Val Thr Val Ser Trp Asn Ser Gly Ala Leu
370 375 380
Thr Ser Gly Val His Thr Phe Pro Ala Val Leu Gln Ser Ser Gly Leu
385 390 395 400
Tyr Ser Leu Ser Ser Val Val Thr Val Pro Ser Ser Ser Leu Gly Thr
405 410 415
Gln Thr Tyr Ile Cys Asn Val Asn His Lys Pro Ser Asn Thr Lys Val
420 425 430
Asp Lys Lys Val Glu Pro Lys Ser Cys Asp Lys Thr His Thr Cys Pro
435 440 445
Pro Cys Pro Ala Pro Glu Ala Ala Gly Gly Pro Ser Val Phe Leu Phe
450 455 460
Pro Pro Lys Pro Lys Asp Thr Leu Met Ile Ser Arg Thr Pro Glu Val
465 470 475 480
Thr Cys Val Val Val Asp Val Ser His Glu Asp Pro Glu Val Lys Phe
485 490 495
Asn Trp Tyr Val Asp Gly Val Glu Val His Asn Ala Lys Thr Lys Pro
500 505 510
Arg Glu Glu Gln Tyr Asn Ser Thr Tyr Arg Val Val Ser Val Leu Thr
515 520 525
Val Leu His Gln Asp Trp Leu Asn Gly Lys Glu Tyr Lys Cys Lys Val
530 535 540
Ser Asn Lys Ala Leu Gly Ala Pro Ile Glu Lys Thr Ile Ser Lys Ala
545 550 555 560
Lys Gly Gln Pro Arg Glu Pro Gln Val Tyr Thr Leu Pro Pro Cys Arg
565 570 575
Asp Glu Leu Thr Lys Asn Gln Val Ser Leu Trp Cys Leu Val Lys Gly
580 585 590
Phe Tyr Pro Ser Asp Ile Ala Val Glu Trp Glu Ser Asn Gly Gln Pro
595 600 605
Glu Asn Asn Tyr Lys Thr Thr Pro Pro Val Leu Asp Ser Asp Gly Ser
610 615 620
Phe Phe Leu Tyr Ser Lys Leu Thr Val Asp Lys Ser Arg Trp Gln Gln
625 630 635 640
Gly Asn Val Phe Ser Cys Ser Val Met His Glu Ala Leu His Asn His
645 650 655
Tyr Thr Gln Lys Ser Leu Ser Leu Ser Pro Gly Lys
660 665

SEQ ID NO: 224
<212> DNA
<223> V9(VL-CH1)-anti-CD33(VH-CH1)-Fc(knob) P329G LALA
gatatccaga tgacccagag ccccagctct ctgagcgcca gcgtgggcga cagagtgacc 60
atcacctgtc gggccagcca ggacatcaga aactacctga actggtatca gcagaagccc 120
ggcaaggccc ccaagctgct gatctactac accagcagac tggaaagcgg cgtgccctcc 180
agattttccg gcagcggctc cggcaccgac tacaccctga ccatcagcag cctgcagccc 240
gaggatttcg ccacatatta ctgccagcag ggcaataccc tgccctggac cttcggacag 300
ggcacaaaag tggaaatcaa gagcagcgct tccaccaaag gcccttccgt gtttcctctg 360
gctcctagct ccaagtccac ctctggaggc accgctgctc tcggatgcct cgtgaaggat 420
tattttcctg agcctgtgac agtgtcctgg aatagcggag cactgacctc tggagtgcat 480
actttccccg ctgtgctgca gtcctctgga ctgtacagcc tgagcagcgt ggtgacagtg 540
cccagcagca gcctgggcac ccagacctac atctgcaacg tgaaccacaa gcccagcaac 600
accaaggtgg acaagaaggt ggaacccaag tcttgtggcg gaggcggatc cggcggaggc 660
ggatctcagg tgcagctggt gcagtctggc gccgaagtga agaaacccgg cagcagcgtg 720
aaggtgtcct gcaaggccag cggctacacc ttcaccgact acaacatgca ctgggtccgc 780
caggccccag gccagggact ggaatggatc ggctacatct acccctacaa cggcggcacc 840
ggctacaacc agaagttcaa gagcaaggcc accatcaccg ccgacgagag caccaacacc 900
gcctacatgg aactgagcag cctgcggagc gaggacaccg ccgtgtacta ctgcgccaga 960
ggcagacccg ccatggacta ctggggccag ggcaccctgg tgacagtgtc cagcgccagc 1020
acaaagggcc ccagcgtgtt ccccctggca cccagcagca agagcacatc tggcggaaca 1080
gccgctctgg gctgtctggt gaaagactac ttccccgagc ccgtgaccgt gtcttggaac 1140
tctggcgccc tgaccagcgg cgtgcacacc tttccagccg tgctgcagag cagcggcctg 1200
tactccctgt cctccgtggt caccgtgccc tctagctccc tgggaacaca gacatatatc 1260
tgtaatgtca atcacaagcc ttccaacacc aaagtcgata agaaagtcga gcccaagagc 1320
tgcgacaaaa ctcacacatg cccaccgtgc ccagcacctg aagctgcagg gggaccgtca 1380
gtcttcctct tccccccaaa acccaaggac accctcatga tctcccggac ccctgaggtc 1440
acatgcgtgg tggtggacgt gagccacgaa gaccctgagg tcaagttcaa ctggtacgtg 1500
gacggcgtgg aggtgcataa tgccaagaca aagccgcggg aggagcagta caacagcacg 1560
taccgtgtgg tcagcgtcct caccgtcctg caccaggact ggctgaatgg caaggagtac 1620
aagtgcaagg tctccaacaa agccctcggc gcccccatcg agaaaaccat ctccaaagcc 1680
aaagggcagc cccgagaacc acaggtgtac accctgcccc catgccggga tgagctgacc 1740
aagaaccagg tcagcctgtg gtgcctggtc aaaggcttct atcccagcga catcgccgtg 1800
gagtgggaga gcaatgggca gccggagaac aactacaaga ccacgcctcc cgtgctggac 1860
tccgacggct ccttcttcct ctacagcaag ctcaccgtgg acaagagcag gtggcagcag 1920
gggaacgtct tctcatgctc cgtgatgcat gaggctctgc acaaccacta cacgcagaag 1980
agcctctccc tgtctccggg taaa 2004

SEQ ID NO: 225
<212> PRT
<223> anti-CD33(VH-CH1)-Fc(hole) P329G LALA
Gln Val Gln Leu Val Gln Ser Gly Ala Glu Val Lys Lys Pro Gly Ser
1 5 10 15
Ser Val Lys Val Ser Cys Lys Ala Ser Gly Tyr Thr Phe Thr Asp Tyr
20 25 30
Asn Met His Trp Val Arg Gln Ala Pro Gly Gln Gly Leu Glu Trp Ile
35 40 45
Gly Tyr Ile Tyr Pro Tyr Asn Gly Gly Thr Gly Tyr Asn Gln Lys Phe
50 55 60
Lys Ser Lys Ala Thr Ile Thr Ala Asp Glu Ser Thr Asn Thr Ala Tyr
65 70 75 80
Met Glu Leu Ser Ser Leu Arg Ser Glu Asp Thr Ala Val Tyr Tyr Cys
85 90 95
Ala Arg Gly Arg Pro Ala Met Asp Tyr Trp Gly Gln Gly Thr Leu Val
100 105 110
Thr Val Ser Ser Ala Ser Thr Lys Gly Pro Ser Val Phe Pro Leu Ala
115 120 125
Pro Ser Ser Lys Ser Thr Ser Gly Gly Thr Ala Ala Leu Gly Cys Leu
130 135 140
Val Lys Asp Tyr Phe Pro Glu Pro Val Thr Val Ser Trp Asn Ser Gly
145 150 155 160
Ala Leu Thr Ser Gly Val His Thr Phe Pro Ala Val Leu Gln Ser Ser
165 170 175
Gly Leu Tyr Ser Leu Ser Ser Val Val Thr Val Pro Ser Ser Ser Leu
180 185 190
Gly Thr Gln Thr Tyr Ile Cys Asn Val Asn His Lys Pro Ser Asn Thr
195 200 205
Lys Val Asp Lys Lys Val Glu Pro Lys Ser Cys Asp Lys Thr His Thr
210 215 220
Cys Pro Pro Cys Pro Ala Pro Glu Ala Ala Gly Gly Pro Ser Val Phe
225 230 235 240
Leu Phe Pro Pro Lys Pro Lys Asp Thr Leu Met Ile Ser Arg Thr Pro
245 250 255
Glu Val Thr Cys Val Val Val Asp Val Ser His Glu Asp Pro Glu Val
260 265 270
Lys Phe Asn Trp Tyr Val Asp Gly Val Glu Val His Asn Ala Lys Thr
275 280 285
Lys Pro Arg Glu Glu Gln Tyr Asn Ser Thr Tyr Arg Val Val Ser Val
290 295 300
Leu Thr Val Leu His Gln Asp Trp Leu Asn Gly Lys Glu Tyr Lys Cys
305 310 315 320
Lys Val Ser Asn Lys Ala Leu Gly Ala Pro Ile Glu Lys Thr Ile Ser
325 330 335
Lys Ala Lys Gly Gln Pro Arg Glu Pro Gln Val Cys Thr Leu Pro Pro
340 345 350
Ser Arg Asp Glu Leu Thr Lys Asn Gln Val Ser Leu Ser Cys Ala Val
355 360 365
Lys Gly Phe Tyr Pro Ser Asp Ile Ala Val Glu Trp Glu Ser Asn Gly
370 375 380
Gln Pro Glu Asn Asn Tyr Lys Thr Thr Pro Pro Val Leu Asp Ser Asp
385 390 395 400
Gly Ser Phe Phe Leu Val Ser Lys Leu Thr Val Asp Lys Ser Arg Trp
405 410 415
Gln Gln Gly Asn Val Phe Ser Cys Ser Val Met His Glu Ala Leu His
420 425 430
Asn His Tyr Thr Gln Lys Ser Leu Ser Leu Ser Pro Gly Lys
435 440 445

SEQ ID NO: 226
<212> DNA
<223> anti-CD33(VH-CH1)-Fc(hole) P329G LALA
caggtgcagc tggtgcagtc tggcgccgaa gtgaagaaac ccggcagcag cgtgaaggtg 60
tcctgcaagg ccagcggcta caccttcacc gactacaaca tgcactgggt ccgccaggcc 120
ccaggccagg gactggaatg gatcggctac atctacccct acaacggcgg caccggctac 180
aaccagaagt tcaagagcaa ggccaccatc accgccgacg agagcaccaa caccgcctac 240
atggaactga gcagcctgcg gagcgaggac accgccgtgt actactgcgc cagaggcaga 300
cccgccatgg actactgggg ccagggcacc ctggtgacag tgtccagcgc tagcaccaag 360
ggcccctccg tgttccccct ggcccccagc agcaagagca ccagcggcgg cacagccgct 420
ctgggctgcc tggtcaagga ctacttcccc gagcccgtga ccgtgtcctg gaacagcgga 480
gccctgacct ccggcgtgca caccttcccc gccgtgctgc agagttctgg cctgtatagc 540
ctgagcagcg tggtcaccgt gccttctagc agcctgggca cccagaccta catctgcaac 600
gtgaaccaca agcccagcaa caccaaggtg gacaagaagg tggagcccaa gagctgcgac 660
aaaactcaca catgcccacc gtgcccagca cctgaagctg cagggggacc gtcagtcttc 720
ctcttccccc caaaacccaa ggacaccctc atgatctccc ggacccctga ggtcacatgc 780
gtggtggtgg acgtgagcca cgaagaccct gaggtcaagt tcaactggta cgtggacggc 840
gtggaggtgc ataatgccaa gacaaagccg cgggaggagc agtacaacag cacgtaccgt 900
gtggtcagcg tcctcaccgt cctgcaccag gactggctga atggcaagga gtacaagtgc 960
aaggtctcca acaaagccct cggcgccccc atcgagaaaa ccatctccaa agccaaaggg 1020
cagccccgag aaccacaggt gtgcaccctg cccccatccc gggatgagct gaccaagaac 1080
caggtcagcc tctcgtgcgc agtcaaaggc ttctatccca gcgacatcgc cgtggagtgg 1140
gagagcaatg ggcagccgga gaacaactac aagaccacgc ctcccgtgct ggactccgac 1200
ggctccttct tcctcgtgag caagctcacc gtggacaaga gcaggtggca gcaggggaac 1260
gtcttctcat gctccgtgat gcatgaggct ctgcacaacc actacacgca gaagagcctc 1320
tccctgtctc cgggtaaa 1338

SEQ ID NO: 227
<212> PRT
<223> anti-CD20(VL-CL)
Asp Ile Val Met Thr Gln Thr Pro Leu Ser Leu Pro Val Thr Pro Gly
1 5 10 15
Glu Pro Ala Ser Ile Ser Cys Arg Ser Ser Lys Ser Leu Leu His Ser
20 25 30
Asn Gly Ile Thr Tyr Leu Tyr Trp Tyr Leu Gln Lys Pro Gly Gln Ser
35 40 45
Pro Gln Leu Leu Ile Tyr Gln Met Ser Asn Leu Val Ser Gly Val Pro
50 55 60
Asp Arg Phe Ser Gly Ser Gly Ser Gly Thr Asp Phe Thr Leu Lys Ile
65 70 75 80
Ser Arg Val Glu Ala Glu Asp Val Gly Val Tyr Tyr Cys Ala Gln Asn
85 90 95
Leu Glu Leu Pro Tyr Thr Phe Gly Gly Gly Thr Lys Val Glu Ile Lys
100 105 110
Arg Thr Val Ala Ala Pro Ser Val Phe Ile Phe Pro Pro Ser Asp Glu
115 120 125
Gln Leu Lys Ser Gly Thr Ala Ser Val Val Cys Leu Leu Asn Asn Phe
130 135 140
Tyr Pro Arg Glu Ala Lys Val Gln Trp Lys Val Asp Asn Ala Leu Gln
145 150 155 160
Ser Gly Asn Ser Gln Glu Ser Val Thr Glu Gln Asp Ser Lys Asp Ser
165 170 175
Thr Tyr Ser Leu Ser Ser Thr Leu Thr Leu Ser Lys Ala Asp Tyr Glu
180 185 190
Lys His Lys Val Tyr Ala Cys Glu Val Thr His Gln Gly Leu Ser Ser
195 200 205
Pro Val Thr Lys Ser Phe Asn Arg Gly Glu Cys
210 215

SEQ ID NO: 228
<212> DNA
<223> anti-CD20(VL-CL)
gatatcgtga tgacccagac tccactctcc ctgcccgtca cccctggaga gcccgccagc 60
attagctgca ggtctagcaa gagcctcttg cacagcaatg gcatcactta tttgtattgg 120
tacctgcaaa agccagggca gtctccacag ctcctgattt atcaaatgtc caaccttgtc 180
tctggcgtcc ctgaccggtt ctccggatcc gggtcaggca ctgatttcac actgaaaatc 240
agcagggtgg aggctgagga tgttggagtt tattactgcg ctcagaatct agaacttcct 300
tacaccttcg gcggagggac caaggtggag atcaaacgta cggtggctgc accatctgtc 360
ttcatcttcc cgccatctga tgagcagttg aaatctggaa ctgcctctgt tgtgtgcctg 420
ctgaataact tctatcccag agaggccaaa gtacagtgga aggtggataa cgccctccaa 480
tcgggtaact cccaggagag tgtcacagag caggacagca aggacagcac ctacagcctc 540
agcagcaccc tgacgctgag caaagcagac tacgagaaac acaaagtcta cgcctgcgaa 600
gtcacccatc agggcctgag ctcgcccgtc acaaagagct tcaacagggg agagtgt 657

SEQ ID NO: 229
<212> PRT
<223> V9(VL-CH1)-anti-CD20(VH-CH1)-Fc(knob) P329G LALA
Asp Ile Gln Met Thr Gln Ser Pro Ser Ser Leu Ser Ala Ser Val Gly
1 5 10 15
Asp Arg Val Thr Ile Thr Cys Arg Ala Ser Gln Asp Ile Arg Asn Tyr
20 25 30
Leu Asn Trp Tyr Gln Gln Lys Pro Gly Lys Ala Pro Lys Leu Leu Ile
35 40 45
Tyr Tyr Thr Ser Arg Leu Glu Ser Gly Val Pro Ser Arg Phe Ser Gly
50 55 60
Ser Gly Ser Gly Thr Asp Tyr Thr Leu Thr Ile Ser Ser Leu Gln Pro
65 70 75 80
Glu Asp Phe Ala Thr Tyr Tyr Cys Gln Gln Gly Asn Thr Leu Pro Trp
85 90 95
Thr Phe Gly Gln Gly Thr Lys Val Glu Ile Lys Ser Ser Ala Ser Thr
100 105 110
Lys Gly Pro Ser Val Phe Pro Leu Ala Pro Ser Ser Lys Ser Thr Ser
115 120 125
Gly Gly Thr Ala Ala Leu Gly Cys Leu Val Lys Asp Tyr Phe Pro Glu
130 135 140
Pro Val Thr Val Ser Trp Asn Ser Gly Ala Leu Thr Ser Gly Val His
145 150 155 160
Thr Phe Pro Ala Val Leu Gln Ser Ser Gly Leu Tyr Ser Leu Ser Ser
165 170 175
Val Val Thr Val Pro Ser Ser Ser Leu Gly Thr Gln Thr Tyr Ile Cys
180 185 190
Asn Val Asn His Lys Pro Ser Asn Thr Lys Val Asp Lys Lys Val Glu
195 200 205
Pro Lys Ser Cys Gly Gly Gly Gly Ser Gly Gly Gly Gly Ser Gln Val
210 215 220
Gln Leu Val Gln Ser Gly Ala Glu Val Lys Lys Pro Gly Ser Ser Val
225 230 235 240
Lys Val Ser Cys Lys Ala Ser Gly Tyr Ala Phe Ser Tyr Ser Trp Ile
245 250 255
Asn Trp Val Arg Gln Ala Pro Gly Gln Gly Leu Glu Trp Met Gly Arg
260 265 270
Ile Phe Pro Gly Asp Gly Asp Thr Asp Tyr Asn Gly Lys Phe Lys Gly
275 280 285
Arg Val Thr Ile Thr Ala Asp Lys Ser Thr Ser Thr Ala Tyr Met Glu
290 295 300
Leu Ser Ser Leu Arg Ser Glu Asp Thr Ala Val Tyr Tyr Cys Ala Arg
305 310 315 320
Asn Val Phe Asp Gly Tyr Trp Leu Val Tyr Trp Gly Gln Gly Thr Leu
325 330 335
Val Thr Val Ser Ser Ala Ser Thr Lys Gly Pro Ser Val Phe Pro Leu
340 345 350
Ala Pro Ser Ser Lys Ser Thr Ser Gly Gly Thr Ala Ala Leu Gly Cys
355 360 365
Leu Val Lys Asp Tyr Phe Pro Glu Pro Val Thr Val Ser Trp Asn Ser
370 375 380
Gly Ala Leu Thr Ser Gly Val His Thr Phe Pro Ala Val Leu Gln Ser
385 390 395 400
Ser Gly Leu Tyr Ser Leu Ser Ser Val Val Thr Val Pro Ser Ser Ser
405 410 415
Leu Gly Thr Gln Thr Tyr Ile Cys Asn Val Asn His Lys Pro Ser Asn
420 425 430
Thr Lys Val Asp Lys Lys Val Glu Pro Lys Ser Cys Asp Lys Thr His
435 440 445
Thr Cys Pro Pro Cys Pro Ala Pro Glu Ala Ala Gly Gly Pro Ser Val
450 455 460
Phe Leu Phe Pro Pro Lys Pro Lys Asp Thr Leu Met Ile Ser Arg Thr
465 470 475 480
Pro Glu Val Thr Cys Val Val Val Asp Val Ser His Glu Asp Pro Glu
485 490 495
Val Lys Phe Asn Trp Tyr Val Asp Gly Val Glu Val His Asn Ala Lys
500 505 510
Thr Lys Pro Arg Glu Glu Gln Tyr Asn Ser Thr Tyr Arg Val Val Ser
515 520 525
Val Leu Thr Val Leu His Gln Asp Trp Leu Asn Gly Lys Glu Tyr Lys
530 535 540
Cys Lys Val Ser Asn Lys Ala Leu Gly Ala Pro Ile Glu Lys Thr Ile
545 550 555 560
Ser Lys Ala Lys Gly Gln Pro Arg Glu Pro Gln Val Tyr Thr Leu Pro
565 570 575
Pro Cys Arg Asp Glu Leu Thr Lys Asn Gln Val Ser Leu Trp Cys Leu
580 585 590
Val Lys Gly Phe Tyr Pro Ser Asp Ile Ala Val Glu Trp Glu Ser Asn
595 600 605
Gly Gln Pro Glu Asn Asn Tyr Lys Thr Thr Pro Pro Val Leu Asp Ser
610 615 620
Asp Gly Ser Phe Phe Leu Tyr Ser Lys Leu Thr Val Asp Lys Ser Arg
625 630 635 640
Trp Gln Gln Gly Asn Val Phe Ser Cys Ser Val Met His Glu Ala Leu
645 650 655
His Asn His Tyr Thr Gln Lys Ser Leu Ser Leu Ser Pro Gly Lys
660 665 670

SEQ ID NO: 230
<212> DNA
<223> V9(VL-CH1)-anti-CD20(VH-CH1)-Fc(knob) P329G LALA
gatatccaga tgacccagag ccccagctct ctgagcgcca gcgtgggcga cagagtgacc 60
atcacctgtc gggccagcca ggacatcaga aactacctga actggtatca gcagaagccc 120
ggcaaggccc ccaagctgct gatctactac accagcagac tggaaagcgg cgtgccctcc 180
agattttccg gcagcggctc cggcaccgac tacaccctga ccatcagcag cctgcagccc 240
gaggatttcg ccacatatta ctgccagcag ggcaataccc tgccctggac cttcggacag 300
ggcacaaaag tggaaatcaa gagcagcgct tccaccaaag gcccttccgt gtttcctctg 360
gctcctagct ccaagtccac ctctggaggc accgctgctc tcggatgcct cgtgaaggat 420
tattttcctg agcctgtgac agtgtcctgg aatagcggag cactgacctc tggagtgcat 480
actttccccg ctgtgctgca gtcctctgga ctgtacagcc tgagcagcgt ggtgacagtg 540
cccagcagca gcctgggcac ccagacctac atctgcaacg tgaaccacaa gcccagcaac 600
accaaggtgg acaagaaggt ggaacccaag tcttgtggcg gaggcggatc cggcggaggg 660
ggatctcagg tgcaattggt gcagtctggc gctgaagtta agaagcctgg gagttcagtg 720
aaggtctcct gcaaggcttc cggatacgcc ttcagctatt cttggatcaa ttgggtgcgg 780
caggcgcctg gacaagggct cgagtggatg ggacggatct ttcccggcga tggggatact 840
gactacaatg ggaaattcaa gggcagagtc acaattaccg ccgacaaatc cactagcaca 900
gcctatatgg agctgagcag cctgagatct gaggacacgg ccgtgtatta ctgtgcaaga 960
aatgtctttg atggttactg gcttgtttac tggggccagg gaaccctggt caccgtctcc 1020
tcagctagca ccaagggccc cagcgtgttc cccctggcac ccagcagcaa gagcacatct 1080
ggcggaacag ccgctctggg ctgtctggtg aaagactact tccccgagcc cgtgaccgtg 1140
tcttggaact ctggcgccct gaccagcggc gtgcacacct ttccagccgt gctgcagagc 1200
agcggcctgt actccctgtc ctccgtggtc accgtgccct ctagctccct gggaacacag 1260
acatatatct gtaatgtcaa tcacaagcct tccaacacca aagtcgataa gaaagtcgag 1320
cccaagagct gcgacaaaac tcacacatgc ccaccgtgcc cagcacctga agctgcaggg 1380
ggaccgtcag tcttcctctt ccccccaaaa cccaaggaca ccctcatgat ctcccggacc 1440
cctgaggtca catgcgtggt ggtggacgtg agccacgaag accctgaggt caagttcaac 1500
tggtacgtgg acggcgtgga ggtgcataat gccaagacaa agccgcggga ggagcagtac 1560
aacagcacgt accgtgtggt cagcgtcctc accgtcctgc accaggactg gctgaatggc 1620
aaggagtaca agtgcaaggt ctccaacaaa gccctcggcg cccccatcga gaaaaccatc 1680
tccaaagcca aagggcagcc ccgagaacca caggtgtaca ccctgccccc atgccgggat 1740
gagctgacca agaaccaggt cagcctgtgg tgcctggtca aaggcttcta tcccagcgac 1800
atcgccgtgg agtgggagag caatgggcag ccggagaaca actacaagac cacgcctccc 1860
gtgctggact ccgacggctc cttcttcctc tacagcaagc tcaccgtgga caagagcagg 1920
tggcagcagg ggaacgtctt ctcatgctcc gtgatgcatg aggctctgca caaccactac 1980
acgcagaaga gcctctccct gtctccgggt aaa 2013

SEQ ID NO: 231
<212> PRT
<223> anti-CD20(VH-CH1)-Fc(hole) P329G LALA
Gln Val Gln Leu Val Gln Ser Gly Ala Glu Val Lys Lys Pro Gly Ser
1 5 10 15
Ser Val Lys Val Ser Cys Lys Ala Ser Gly Tyr Ala Phe Ser Tyr Ser
20 25 30
Trp Ile Asn Trp Val Arg Gln Ala Pro Gly Gln Gly Leu Glu Trp Met
35 40 45
Gly Arg Ile Phe Pro Gly Asp Gly Asp Thr Asp Tyr Asn Gly Lys Phe
50 55 60
Lys Gly Arg Val Thr Ile Thr Ala Asp Lys Ser Thr Ser Thr Ala Tyr
65 70 75 80
Met Glu Leu Ser Ser Leu Arg Ser Glu Asp Thr Ala Val Tyr Tyr Cys
85 90 95
Ala Arg Asn Val Phe Asp Gly Tyr Trp Leu Val Tyr Trp Gly Gln Gly
100 105 110
Thr Leu Val Thr Val Ser Ser Ala Ser Thr Lys Gly Pro Ser Val Phe
115 120 125
Pro Leu Ala Pro Ser Ser Lys Ser Thr Ser Gly Gly Thr Ala Ala Leu
130 135 140
Gly Cys Leu Val Lys Asp Tyr Phe Pro Glu Pro Val Thr Val Ser Trp
145 150 155 160
Asn Ser Gly Ala Leu Thr Ser Gly Val His Thr Phe Pro Ala Val Leu
165 170 175
Gln Ser Ser Gly Leu Tyr Ser Leu Ser Ser Val Val Thr Val Pro Ser
180 185 190
Ser Ser Leu Gly Thr Gln Thr Tyr Ile Cys Asn Val Asn His Lys Pro
195 200 205
Ser Asn Thr Lys Val Asp Lys Lys Val Glu Pro Lys Ser Cys Asp Lys
210 215 220
Thr His Thr Cys Pro Pro Cys Pro Ala Pro Glu Ala Ala Gly Gly Pro
225 230 235 240
Ser Val Phe Leu Phe Pro Pro Lys Pro Lys Asp Thr Leu Met Ile Ser
245 250 255
Arg Thr Pro Glu Val Thr Cys Val Val Val Asp Val Ser His Glu Asp
260 265 270
Pro Glu Val Lys Phe Asn Trp Tyr Val Asp Gly Val Glu Val His Asn
275 280 285
Ala Lys Thr Lys Pro Arg Glu Glu Gln Tyr Asn Ser Thr Tyr Arg Val
290 295 300
Val Ser Val Leu Thr Val Leu His Gln Asp Trp Leu Asn Gly Lys Glu
305 310 315 320
Tyr Lys Cys Lys Val Ser Asn Lys Ala Leu Gly Ala Pro Ile Glu Lys
325 330 335
Thr Ile Ser Lys Ala Lys Gly Gln Pro Arg Glu Pro Gln Val Cys Thr
340 345 350
Leu Pro Pro Ser Arg Asp Glu Leu Thr Lys Asn Gln Val Ser Leu Ser
355 360 365
Cys Ala Val Lys Gly Phe Tyr Pro Ser Asp Ile Ala Val Glu Trp Glu
370 375 380
Ser Asn Gly Gln Pro Glu Asn Asn Tyr Lys Thr Thr Pro Pro Val Leu
385 390 395 400
Asp Ser Asp Gly Ser Phe Phe Leu Val Ser Lys Leu Thr Val Asp Lys
405 410 415
Ser Arg Trp Gln Gln Gly Asn Val Phe Ser Cys Ser Val Met His Glu
420 425 430
Ala Leu His Asn His Tyr Thr Gln Lys Ser Leu Ser Leu Ser Pro Gly
435 440 445
Lys

SEQ ID NO: 232
<212> DNA
<223> anti-CD20(VH-CH1)-Fc(hole) P329G LALA
caggtgcaat tggtgcagtc tggcgctgaa gttaagaagc ctgggagttc agtgaaggtc 60
tcctgcaagg cttccggata cgccttcagc tattcttgga tcaattgggt gcggcaggcg 120
cctggacaag ggctcgagtg gatgggacgg atctttcccg gcgatgggga tactgactac 180
aatgggaaat tcaagggcag agtcacaatt accgccgaca aatccactag cacagcctat 240
atggagctga gcagcctgag atctgaggac acggccgtgt attactgtgc aagaaatgtc 300
tttgatggtt actggcttgt ttactggggc cagggaaccc tggtcaccgt ctcctcagct 360
agcaccaagg gcccatcggt cttccccctg gcaccctcct ccaagagcac ctctgggggc 420
acagcggccc tgggctgcct ggtcaaggac tacttccccg aaccggtgac ggtgtcgtgg 480
aactcaggcg ccctgaccag cggcgtgcac accttcccgg ctgtcctaca gtcctcagga 540
ctctactccc tcagcagcgt ggtgaccgtg ccctccagca gcttgggcac ccagacctac 600
atctgcaacg tgaatcacaa gcccagcaac accaaggtgg acaagaaagt tgagcccaaa 660
tcttgtgaca aaactcacac atgcccaccg tgcccagcac ctgaagctgc agggggaccg 720
tcagtcttcc tcttcccccc aaaacccaag gacaccctca tgatctcccg gacccctgag 780
gtcacatgcg tggtggtgga cgtgagccac gaagaccctg aggtcaagtt caactggtac 840
gtggacggcg tggaggtgca taatgccaag acaaagccgc gggaggagca gtacaacagc 900
acgtaccgtg tggtcagcgt cctcaccgtc ctgcaccagg actggctgaa tggcaaggag 960
tacaagtgca aggtctccaa caaagccctc ggcgccccca tcgagaaaac catctccaaa 1020
gccaaagggc agccccgaga accacaggtg tgcaccctgc ccccatcccg ggatgagctg 1080
accaagaacc aggtcagcct ctcgtgcgca gtcaaaggct tctatcccag cgacatcgcc 1140
gtggagtggg agagcaatgg gcagccggag aacaactaca agaccacgcc tcccgtgctg 1200
gactccgacg gctccttctt cctcgtgagc aagctcaccg tggacaagag caggtggcag 1260
caggggaacg tcttctcatg ctccgtgatg catgaggctc tgcacaacca ctacacgcag 1320
aagagcctct ccctgtctcc gggtaaa 1347

SEQ ID NO: 233
<212> PRT
<223> M4-3 ML2 HCDR1
Gly Gly Ser Ile Thr Ser Gly Tyr Tyr Trp Asn
1 5 10

SEQ ID NO: 234
<212> DNA
<223> M4-3 ML2 HCDR1
ggcggcagca tcaccagcgg ctactactgg aac 33

SEQ ID NO: 235
<212> PRT
<223> M4-3 ML2 HCDR2
Tyr Ile Thr Tyr Asp Gly Ser Asn Asn Tyr Asn Pro Ser Leu Lys Ser
1 5 10 15

SEQ ID NO: 236
<212> DNA
<223> M4-3 ML2 HCDR2
tacatcacct acgacggcag caacaactac aaccccagcc tgaagtcc 48

SEQ ID NO: 237
<212> PRT
<223> M4-3 ML2 HCDR3
Phe Asp Tyr
1

SEQ ID NO: 238
<212> DNA
<223> M4-3 ML2 HCDR3
ttcgactac 9

SEQ ID NO: 239
<212> PRT
<223> M4-3 ML2 VH
Gln Val Gln Leu Gln Glu Ser Gly Pro Gly Leu Val Lys Pro Ser Gln
1 5 10 15
Thr Leu Ser Leu Thr Cys Thr Val Ser Gly Gly Ser Ile Thr Ser Gly
20 25 30
Tyr Tyr Trp Asn Trp Ile Arg Gln His Pro Gly Lys Gly Leu Glu Trp
35 40 45
Ile Gly Tyr Ile Thr Tyr Asp Gly Ser Asn Asn Tyr Asn Pro Ser Leu
50 55 60
Lys Ser Arg Val Thr Ile Ser Arg Asp Thr Ser Lys Asn Gln Phe Ser
65 70 75 80
Leu Lys Leu Ser Ser Val Thr Ala Ala Asp Thr Ala Val Tyr Tyr Cys
85 90 95
Ala Asp Phe Asp Tyr Trp Gly Gln Gly Thr Leu Val Thr Val Ser Ser
100 105 110

SEQ ID NO: 240
<212> DNA
<223> M4-3 ML2 VH
caggtgcagc tgcaggaaag cggccctggc ctggtcaagc ccagccagac cctgagcctg 60
acctgcaccg tgtccggcgg cagcatcacc agcggctact actggaactg gatccggcag 120
caccccggca agggcctgga atggatcggc tacatcacct acgacggcag caacaactac 180
aaccccagcc tgaagtccag agtgaccatc agccgggaca ccagcaagaa ccagttcagc 240
ctgaagctgt ccagcgtgac agccgccgac accgccgtgt actactgcgc cgacttcgac 300
tactggggcc agggcaccct ggtcaccgtg tccagc 336

SEQ ID NO: 241
<212> PRT
<223> M4-3 ML2 LCDR1
Arg Ala Ser Gln Gly Ile Arg Asn Tyr Leu Asn
1 5 10

SEQ ID NO: 242
<212> DNA
<223> M4-3 ML2 LCDR1
cgggccagcc agggcatccg gaactacctg aac 33

SEQ ID NO: 243
<212> PRT
<223> M4-3 ML2 LCDR2
Tyr Thr Ser Ser Leu His Ser
1 5

SEQ ID NO: 244
<212> DNA
<223> M4-3 ML2 LCDR2
tacaccagca gcctgcacag c 21

SEQ ID NO: 245
<212> PRT
<223> M4-3 ML2 LCDR3
Gln Gln Tyr Ser Lys Leu Pro Trp Thr
1 5

SEQ ID NO: 246
<212> DNA
<223> M4-3 ML2 LCDR3
cagcagtaca gcaagctgcc ctggacc 27

SEQ ID NO: 247
<212> PRT
<223> M4-3 ML2 VL
Asp Ile Gln Met Thr Gln Ser Pro Ser Ser Leu Ser Ala Ser Val Gly
1 5 10 15
Asp Arg Val Thr Ile Thr Cys Arg Ala Ser Gln Gly Ile Arg Asn Tyr
20 25 30
Leu Asn Trp Tyr Gln Gln Lys Pro Gly Lys Ala Pro Lys Leu Leu Ile
35 40 45
Tyr Tyr Thr Ser Ser Leu His Ser Gly Val Pro Ser Arg Phe Ser Gly
50 55 60
Ser Gly Ser Gly Thr Asp Phe Thr Leu Thr Ile Ser Ser Leu Gln Pro
65 70 75 80
Glu Asp Phe Ala Thr Tyr Tyr Cys Gln Gln Tyr Ser Lys Leu Pro Trp
85 90 95
Thr Phe Gly Gln Gly Thr Lys Val Glu Ile Lys
100 105

SEQ ID NO: 248
<212> DNA
<223> M4-3 ML2 VL
gacatccaga tgacccagag ccccagcagc ctgagcgcca gcgtgggcga cagagtgacc 60
atcacctgcc gggccagcca gggcatccgg aactacctga actggtatca gcagaagccc 120
ggcaaggccc ccaagctgct gatctactac accagcagcc tgcacagcgg cgtgcctagc 180
cggtttagcg gcagcggctc cggcaccgac ttcaccctga ccattagctc cctgcagccc 240
gaggacttcg ccacctacta ctgccagcag tacagcaagc tgccctggac cttcggccag 300
ggaacaaagg tggagatcaa g 321

SEQ ID NO: 249
<212> PRT
<223> anti-CD3 HCDR1
Thr Tyr Ala Met Asn
1 5

SEQ ID NO: 250
<212> DNA
<223> anti-CD3 HCDR1
acctacgcca tgaac 15

SEQ ID NO: 251
<212> PRT
<223> anti-CD3 HCDR2
Arg Ile Arg Ser Lys Tyr Asn Asn Tyr Ala Thr Tyr Tyr Ala Asp Ser
1 5 10 15
Val Lys Asp

SEQ ID NO: 252
<212> DNA
<223> anti-CD3 HCDR2
cggatcagaa gcaagtacaa caattacgcc acctactacg ccgacagcgt gaaggac 57

SEQ ID NO: 253
<212> PRT
<223> anti-CD3 HCDR3
His Gly Asn Phe Gly Asn Ser Tyr Val Ser Trp Phe Ala Tyr
1 5 10

SEQ ID NO: 254
<212> DNA
<223> anti-CD3 HCDR3
cacggcaact tcggcaacag ctatgtgtct tggtttgcct ac 42

SEQ ID NO: 255
<212> PRT
<223> anti-CD3 VH
Glu Val Lys Leu Leu Glu Ser Gly Gly Gly Leu Val Gln Pro Lys Gly
1 5 10 15
Ser Leu Lys Leu Ser Cys Ala Ala Ser Gly Phe Thr Phe Asn Thr Tyr
20 25 30
Ala Met Asn Trp Val Arg Gln Ala Pro Gly Lys Gly Leu Glu Trp Val
35 40 45
Ala Arg Ile Arg Ser Lys Tyr Asn Asn Tyr Ala Thr Tyr Tyr Ala Asp
50 55 60
Ser Val Lys Asp Arg Phe Thr Ile Ser Arg Asp Asp Ser Gln Ser Ile
65 70 75 80
Leu Tyr Leu Gln Met Asn Asn Leu Lys Thr Glu Asp Thr Ala Met Tyr
85 90 95
Tyr Cys Val Arg His Gly Asn Phe Gly Asn Ser Tyr Val Ser Trp Phe
100 105 110
Ala Tyr Trp Gly Gln Gly Thr Leu Val Thr Val Ser Ala
115 120 125

SEQ ID NO: 256
<212> DNA
<223> anti-CD3 VH
gaagtgcagc tggtggaaag cggcggaggc ctggtgcagc ctaagggctc tctgaagctg 60
agctgtgccg ccagcggctt caccttcaac acctacgcca tgaactgggt gcgccaggcc 120
cctggcaaag gcctggaatg ggtggcccgg atcagaagca agtacaacaa ttacgccacc 180
tactacgccg acagcgtgaa ggaccggttc accatcagcc gggacgacag ccagagcatc 240
ctgtacctgc agatgaacaa cctgaaaacc gaggacaccg ccatgtacta ctgcgtgcgg 300
cacggcaact tcggcaacag ctatgtgtct tggtttgcct actggggcca gggcaccctc 360
gtgacagtgt ctgct 375

SEQ ID NO: 257
<212> PRT
<223> anti-CD3 LCDR1
Arg Ser Ser Thr Gly Ala Val Thr Thr Ser Asn Tyr Ala Asn
1 5 10

SEQ ID NO: 258
<212> DNA
<223> anti-CD3 LCDR1
agatctagca caggcgccgt gaccaccagc aactacgcca ac 42

SEQ ID NO: 259
<212> PRT
<223> anti-CD3 LCDR2
Gly Thr Asn Lys Arg Ala Pro
1 5

SEQ ID NO: 260
<212> DNA
<223> anti-CD3 LCDR2
ggcaccaaca aaagggctcc a 21

SEQ ID NO: 261
<212> PRT
<223> anti-CD3 LCDR3
Ala Leu Trp Tyr Ser Asn Leu Trp Val
1 5

SEQ ID NO: 262
<212> DNA
<223> anti-CD3 LCDR3
gccctgtggt acagcaacct gtgggtg 27

SEQ ID NO: 263
<212> PRT
<223> anti-CD3 VL
Gln Ala Val Val Thr Gln Glu Ser Ala Leu Thr Thr Ser Pro Gly Glu
1 5 10 15
Thr Val Thr Leu Thr Cys Arg Ser Ser Thr Gly Ala Val Thr Thr Ser
20 25 30
Asn Tyr Ala Asn Trp Val Gln Glu Lys Pro Asp His Leu Phe Thr Gly
35 40 45
Leu Ile Gly Gly Thr Asn Lys Arg Ala Pro Gly Val Pro Ala Arg Phe
50 55 60
Ser Gly Ser Leu Ile Gly Asp Lys Ala Ala Leu Thr Ile Thr Gly Ala
65 70 75 80
Gln Thr Glu Asp Glu Ala Ile Tyr Phe Cys Ala Leu Trp Tyr Ser Asn
85 90 95
Leu Trp Val Phe Gly Gly Gly Thr Lys Leu Thr Val Leu
100 105

SEQ ID NO: 264
<212> DNA
<223> anti-CD3 VL
caggccgtcg tgacccagga aagcgccctg acaacaagcc ctggcgagac agtgaccctg 60
acctgcagat ctagcacagg cgccgtgacc accagcaact acgccaactg ggtgcaggaa 120
aagcccgacc acctgttcac cggcctgatc ggcggcacca acaaaagggc tccaggcgtg 180
ccagccagat tcagcggcag cctgattggc gataaggccg ccctgaccat cactggcgcc 240
cagacagagg acgaggccat ctacttttgc gccctgtggt acagcaacct gtgggtgttc 300
ggcggaggca ccaagctgac agtgctg 327

SEQ ID NO: 265
<212> PRT
Met Gln Ser Gly Thr His Trp Arg Val Leu Gly Leu Cys Leu Leu Ser
1 5 10 15
Val Gly Val Trp Gly Gln Asp Gly Asn Glu Glu Met Gly Gly Ile Thr
20 25 30
Gln Thr Pro Tyr Lys Val Ser Ile Ser Gly Thr Thr Val Ile Leu Thr
35 40 45
Cys Pro Gln Tyr Pro Gly Ser Glu Ile Leu Trp Gln His Asn Asp Lys
50 55 60
Asn Ile Gly Gly Asp Glu Asp Asp Lys Asn Ile Gly Ser Asp Glu Asp
65 70 75 80
His Leu Ser Leu Lys Glu Phe Ser Glu Leu Glu Gln Ser Gly Tyr Tyr
85 90 95
Val Cys Tyr Pro Arg Gly Ser Lys Pro Glu Asp Ala Asn Phe Tyr Leu
100 105 110
Tyr Leu Arg Ala Arg Val Cys Glu Asn Cys Met Glu Met Asp Val Met
115 120 125
Ser Val Ala Thr Ile Val Ile Val Asp Ile Cys Ile Thr Gly Gly Leu
130 135 140
Leu Leu Leu Val Tyr Tyr Trp Ser Lys Asn Arg Lys Ala Lys Ala Lys
145 150 155 160
Pro Val Thr Arg Gly Ala Gly Ala Gly Gly Arg Gln Arg Gly Gln Asn
165 170 175
Lys Glu Arg Pro Pro Pro Val Pro Asn Pro Asp Tyr Glu Pro Ile Arg
180 185 190
Lys Gly Gln Arg Asp Leu Tyr Ser Gly Leu Asn Gln Arg Arg Ile
195 200 205

SEQ ID NO: 266
<212> PRT
Met Gln Ser Gly Thr Arg Trp Arg Val Leu Gly Leu Cys Leu Leu Ser
1 5 10 15
Ile Gly Val Trp Gly Gln Asp Gly Asn Glu Glu Met Gly Ser Ile Thr
20 25 30
Gln Thr Pro Tyr Gln Val Ser Ile Ser Gly Thr Thr Val Ile Leu Thr
35 40 45
Cys Ser Gln His Leu Gly Ser Glu Ala Gln Trp Gln His Asn Gly Lys
50 55 60
Asn Lys Glu Asp Ser Gly Asp Arg Leu Phe Leu Pro Glu Phe Ser Glu
65 70 75 80
Met Glu Gln Ser Gly Tyr Tyr Val Cys Tyr Pro Arg Gly Ser Asn Pro
85 90 95
Glu Asp Ala Ser His His Leu Tyr Leu Lys Ala Arg Val Cys Glu Asn
100 105 110
Cys Met Glu Met Asp Val Met Ala Val Ala Thr Ile Val Ile Val Asp
115 120 125
Ile Cys Ile Thr Leu Gly Leu Leu Leu Leu Val Tyr Tyr Trp Ser Lys
130 135 140
Asn Arg Lys Ala Lys Ala Lys Pro Val Thr Arg Gly Ala Gly Ala Gly
145 150 155 160
Gly Arg Gln Arg Gly Gln Asn Lys Glu Arg Pro Pro Pro Val Pro Asn
165 170 175
Pro Asp Tyr Glu Pro Ile Arg Lys Gly Gln Gln Asp Leu Tyr Ser Gly
180 185 190
Leu Asn Gln Arg Arg Ile
195

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1. A T cell activating bispecific antigen binding molecule comprising a first and a second antigen binding moiety, one of which is a Fab molecule capable of specific binding to an activating T cell antigen and the other one of which is a Fab molecule capable of specific binding to a target cell antigen, and an Fc domain composed of a first and a second subunit capable of stable association; wherein the first antigen binding moiety is
(a) a single chain Fab molecule wherein the Fab light chain and the Fab heavy chain are connected by a peptide linker, or
(b) a crossover Fab molecule wherein either the variable or the constant regions of the Fab light chain and the Fab heavy chain are exchanged.

2. The T cell activating bispecific antigen binding molecule of claim 1, comprising not more than one antigen binding moiety capable of specific binding to an activating T cell antigen.

3. The T cell activating bispecific antigen binding molecule of claim 1, wherein the first and the second antigen binding moiety are fused to each other, optionally via a peptide linker.

4. The T cell activating bispecific antigen binding molecule of claim 1, wherein the second antigen binding moiety is fused at the C-terminus of the Fab heavy chain to the N-terminus of the Fab heavy chain of the first antigen binding moiety.

5. The T cell activating bispecific antigen binding molecule of claim 1, wherein the first antigen binding moiety is fused at the C-terminus of the Fab heavy chain to the N-terminus of the Fab heavy chain of the second antigen binding moiety.

6. The T cell activating bispecific antigen binding molecule of claim 4 or 5, wherein the first antigen binding moiety is a crossover Fab molecule and the Fab light chain of the first antigen binding moiety and the Fab light chain of the second antigen binding moiety are fused to each other, optionally via a peptide linker.

7. The T cell activating bispecific antigen binding molecule of claim 1, wherein the second antigen binding moiety of the T cell activating bispecific antigen binding molecule is fused at the C-terminus of the Fab light chain to the N-terminus of the Fab light chain of the first antigen binding moiety.

8. The T cell activating bispecific antigen binding molecule of claim 1, wherein the second antigen binding moiety of the T cell activating bispecific antigen binding molecule is fused at the C-terminus of the Fab heavy chain to the N-terminus of the first or the second subunit of the Fc domain.

9. The T cell activating bispecific antigen binding molecule of claim 1, wherein the first antigen binding moiety is fused at the C-terminus of the Fab heavy chain to the N-terminus of the first or second subunit of the Fc domain.

10. The T cell activating bispecific antigen binding molecule of claim 1, wherein the first and the second antigen binding moiety are each fused at the C-terminus of the Fab heavy chain to the N-terminus of one of the subunits of the Fc domain.

11. The T cell activating bispecific antigen binding molecule of claim 1, comprising a third antigen binding moiety which is a Fab molecule capable of specific binding to a target cell antigen.

12. The T cell activating bispecific antigen binding molecule of claim 11, wherein the third antigen binding moiety is fused at the C-terminus of the Fab heavy chain to the N-terminus of the first or second subunit of the Fc domain.

13. The T cell activating bispecific antigen binding molecule of claim 11 or 12, wherein the second and the third antigen binding moiety are each fused at the C-terminus of the Fab heavy chain to the N-terminus of one of the subunits of the Fc domain, and the first antigen binding moiety is fused at the C-terminus of the Fab heavy chain to the N-terminus of the Fab heavy chain of the second antigen binding moiety.

14. The T cell activating bispecific antigen binding molecule of claim 11 or 12, wherein the first and the third antigen binding moiety are each fused at the C-terminus of the Fab heavy chain to the N-terminus of one of the subunits of the Fc domain, and the second antigen binding moiety is fused at the C-terminus of the Fab heavy chain to the N-terminus of the Fab heavy chain of the first antigen binding moiety.

15. The T cell activating bispecific antigen binding molecule of claim 13, wherein the second and the third antigen binding moiety and the Fc domain are part of an immunoglobulin molecule, particularly an IgG class immunoglobulin.

16. The T cell activating bispecific antigen binding molecule of claim 1, wherein the Fc domain is an IgG, specifically an IgG₁ or IgG₄, Fc domain.

17. The T cell activating bispecific antigen binding molecule of claim 1, wherein the Fc domain is a human Fc domain.

18. The T cell activating bispecific antigen binding molecule of claim 1, wherein the Fc domain comprises a modification promoting the association of the first and the second subunit of the Fc domain.

19. The T cell activating bispecific antigen binding molecule of claim 18, wherein in the CH3 domain of the first subunit of the Fc domain an amino acid residue is replaced with an amino acid residue having a larger side chain volume, thereby generating a protuberance within the CH3 domain of the first subunit which is positionable in a cavity within the CH3 domain of the second subunit, and in the CH3 domain of the second subunit of the Fc domain an amino acid residue is replaced with an amino acid residue having a smaller side chain volume, thereby generating a cavity within the CH3 domain of the second subunit within which the protuberance within the CH3 domain of the first subunit is positionable.

20. The T cell activating bispecific antigen binding molecule of claim 1, wherein the Fc domain exhibits reduced binding affinity to an Fc receptor and/or reduced effector function, as compared to a native IgG1 Fc domain.

21. The T cell activating bispecific antigen binding molecule of claim 1, wherein the Fc domain comprises one or more amino acid substitution that reduces binding to an Fc receptor and/or effector function.

22. The T cell activating bispecific antigen binding molecule of claim 21, wherein said one or more amino acid substitution is at one or more position selected from the group of L234, L235, and P329.

23. The T cell activating bispecific antigen binding molecule of claim 22, wherein each subunit of the Fc domain comprises three amino acid substitutions that reduce binding to an activating Fc receptor and/or effector function wherein said amino acid substitutions are L234A, L235A and P329G.

24. The T cell activating bispecific antigen binding molecule of claim 20 or claim 21, wherein the Fc receptor is an Fcγ receptor.

25. The T cell activating bispecific antigen binding molecule of claim 20 or claim 21, wherein the effector function is antibody-dependent cell-mediated cytotoxicity (ADCC).

26. The T cell activating bispecific antigen binding molecule of claim 1, wherein the activating T cell antigen is CD3.

27. The T cell activating bispecific antigen binding molecule of any one of the preceding claims, wherein the target cell antigen is selected from the group consisting of: Melanoma-associated Chondroitin Sulfate Proteoglycan (MCSP), Epidermal Growth Factor Receptor (EGFR), CD19, CD20, CD33, Carcinoembryonic Antigen (CEA) and Fibroblast Activation Protein (FAP).

28. An isolated polynucleotide encoding the T cell activating bispecific antigen binding molecule of claim 1 or a fragment thereof.

29. A vector comprising the isolated polynucleotide of claim 28.

30. A host cell comprising the polynucleotide of claim 28.

31. A method of producing the T cell activating bispecific antigen binding molecule of claim 1, comprising the steps of a) culturing the host cell of claim 30 under conditions suitable for the expression of the T cell activating bispecific antigen binding molecule and b) recovering the T cell activating bispecific antigen binding molecule.

32. A pharmaceutical composition comprising the T cell activating bispecific antigen binding molecule of claim 1 and a pharmaceutically acceptable carrier.

33. A method of treating a disease in an individual, comprising administering to said individual a therapeutically effective amount of a composition comprising the T cell activating bispecific antigen binding molecule of claim 1 in a pharmaceutically acceptable form.

34. The method of claim 33, wherein said disease is cancer.

35. A method for inducing lysis of a target cell, comprising contacting a target cell with the T cell activating bispecific antigen binding molecule of claim 1 in the presence of a T cell.
